SEQUENCE LISTING (716 sequences)

SEQ ID NO: 1 - protein, 412 aa, Homo sapiens
MSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAK

SEQ ID NO: 2 - DNA, 1863 bp, Homo sapiens
  source 1..1863: mol_type = other DNA
gccggcgcgcccctgggagggtgagccggcgccgggcccaggcccggacctggtgggaggcggggggaggtggggacgaggcctggggaggcgggccccgcccatctgcaggtggctgtgaacgctgagcggctccaggcgggggccgggcccgggggcggggtctgtggcgcgcgtccccgccacgtgtccccggtcaccggccctgcccccgggccctgtgcttataacctgggatgggcacccctgccagtcctgctctgccgcctgccaccgctgcccgagcccgagtggttcactgcactgtgaaaacagattccagacgccgggaactcacgcctccaatcccagacgctatgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatagacccgtgtacaatgaggagctggggcctcctcaatttgcagatcccccaagtacaggcgctaattgttgtgataatttgtaattgtgacttgttctccccggctggcagcgtagtggggctgccaggccccagctttgttccctggtccccctgaagcctgcaaacgttgtcatcgaagggaagggtggggggcagctgcggtggggagctataaaaatgacaattaaaagagacactagtcttttatttctaaaaaaaaaaaaaaaa

SEQ ID NO: 3 - DNA, 1239 bp, Homo sapiens
  source 1..1239: mol_type = other DNA
atgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatag

SEQ ID NO: 4 - DNA, 4027 bp, Homo sapiens
  source 1..4027: mol_type = other DNA
gtaccaggcgggaggcagggatttgggctgggagtggggcggtgatgtggagggcagtggtggatgctcctgccccagggatcccatcccttccagtgtgtcttcttgcttctaagagtatgagatcatcctgctctcaggtgtgtgaaccctcttgtttttctgcctcccccacgccctcttccacccacccaccttcccatccaggtggagtgtctcttccccacccacataccttcccttctcttttatctctctgtccatctatgcacccactcaccctcttttatctctgtttatctactcgtccacatatccatccatcattcatccctccattagctatccatcgatccatccatccatccattcagccatccatccatccatcacccacccatccattcacccatcatccatccatccatccatccatcacccacccatccattcacccatcatccatccatccatccattcatccattcatccatcatccatccacccatccatccatcacccatccactcatccacccatccattatccatccatccattcatccatccatccatcatccatctatccatctatccatccatcctccgtccattcatccatcatccatccatccattcacccatcatccatccatccatcatccatccattcatccatcatccatccatccatccattcacccatcatccattcatccagccatcatccgtctatccatccatcatccatttattcatccatcatccatccatccatccatcattcttcatccttccatgcatcatccattcatccatccatcatctacccatgcatccatccatcatccatccgtctattcatccatccattcatccatccatcatctatccatccatcccattatccattcacccatccatcccttcattatccatccatccatccacatatccatctgtcattaatccctccactgtctatccatccattcatccagacagccagccattcatctatccatcccccatccatccatccatccttcatccattcaccgtttttctatccatccattcatccatccatccatctgtcatccatcatccatccttctgtccatcatccccctacctgtccttccctcttttatctctctgtctacccattcttccattcattcaacagaatcagagaactccaattgagtctggtgccttgattgtgaatctaggcatgtggcaggcagacccctgtcccatacagggcatttatgatgaacagtgtcatcaggacccctgctcacatgtgctcactccccatggagaagcccttgccacttgtgccccttcatttgggagagtgagcaggggacatagcagggatgatgtgcaaggccaccagcccacctgcagggtcagagctgatcccctctgtggggtgcttctgcagggttgccagggtaattagcatgttaaaatgaacacgggagggagtggatgccccagtcccaagtcacctactggccacaggcctggaactagaatgtccctccttggttctgtgcctcccgagtgctggagagcagaggggttgtctgagcagtagtccaggaaggggagaaagagccctgagtgttgaagattcgagtgcgctttcagcagcgcaggagcaggctttgtgcacggcttcagcttttgtcctttattctagatgagcagttctcaaaacttgtggtcttgggaatcctctacactctaatttacgtggcgtttttaatgtttattttcttgcatataacattaggatgggccaggcacagtggctcacgcctgtaatcccagcactttggcaggctgaggcaggcagatcacctgaggtcaggagttcaagaccagcctggccaacatggtgaaaccctgtctctactaaaaatacaaaaattagctgggcgcgatggtgggtgcctgtaatcccagatgctcgggaggctgaggcaggagagtcgcttgaacccaggaggcggaggttgcagtaagctgaaatcacaccattgcactccatcctgggcaacaacagcaaaactctgtaaaaaaaaccaaccaaccaaccaaacaaacacaaaaacacatttatcctaatgttttttatatatccttagtcccaattgctttcttttctatgcctttttatttcattttttaaaaggatttttttcaaaataattatagacacaggaagttgcaaaagcagtaccaagaatttctcgactcttaaaaattattgaaggccttggctgggtgcggtggctcacgcttgtaatcctagcactttgggaggctgaagtgggcagatctcttgaggtcaggagtttgagaccagcctggccaaccagcctagtttctttagtagagaaaccccacatgcctgtagtcccagccactcgggaggctgaggcaggagaatcacttgaacccggaaggcagaggttacagtgagccaagattgcacctctgcactccagtctggatgacagagagtccatctcaaaaaaataaaaattactgaaggccttagagagcttttgtggattacatctgttgatatttatcacattagaaataaaagctgaaatgttttaaatgaacagttcatgttaacataagtaacattacacttatgaaaaataactattttctcaaacaaaaaattttagttaagaatcttattggcttccatttttgcaaatctctgtaatgtctggcttgacagaaaacagatggactcttgtagcggttttctgcattcacttgttttttcgtttgtttgtttgtttttcttttttagcctcccagggcttcctcgctatgtgatgccttgttttattgaagtttatgaagaaaactgggcaacacagtgagatcccatctctacaaaaaataaaaaacttagccgggcgtggtagcacatgcccgtagtcccagctactcaagaggctgaggtgggaggattgcttgaacccaggaggttgaggctgcagtgagctaggatcacaccactgcactccagtctggatgacagagaaagaccctgtctcaaaaaaacaaaaaaaaagttttcactcagtgtctccctgaaaagtcattgggaacccctgggggtctacatttcaagaaccattcctctggatcggggtttctcagtcttggccttgttcccgtgtggctggccgtcctgtgcacgggaggattttagcagtatccccatagcctttacccactagatgccagaattccaccccccacctggtagttgtggcaaccaaaaatatctccagatacttccaaatgcctcccctactcccacccccaacagggacaaaaccgtctctggttgggaactactgctgtgagatcttaggtgacaactaagtttcatcttgttggggctgatttagaagccacatccagtttcacagaacggtgcatcaggattgattagcaggctggccgcggtggcttgtgcctgtaatcccagcactttgggaggccgaggcagctggatcacctgaggtcaggggttcaagaccaacctggccaacatggtgaaaccccgtctctaccaaaaatacaaaaattagcaagccatggtggcatgcgcctgtaatcccagctactcaagaggctgaggcaggggagttgcttgaacctgggagacggaggttgcagtgagttgagactgagccactgcactccaacctgggcaacagagcaagacttcgactcaaaaaaaaaaaaaaaaagagagctattgattagcaatgtctgccatgagtgcaggagggagagtaacagtgcagtgtgctgggattgatgaatgatgtctactacagatgcagaagaagaagcaccaggtacagcgggggtggccccgtataggtctcagctagctgaggcccctggggctctgtatgccagatggcccctgtccttgcctacttcttccttctgggctcctcttcccgtag

SEQ ID NO: 5 - DNA, 1939 bp, Mus musculus
  source 1..1939: mol_type = other DNA
gtgaggaagtgccttgcgtgtccacccctcccccattgctcccctcactgcccacacgtcaccgtcctccgccaagccttgcttttttctctctctacaatcctcctgagacagaagggaaacagcctctccctccatgagacgcctccattctttgaccagagagggctggctctcaggaaggtgataaaggatgaaggccaccaggcagatccttagcttgccactgctctgtctagaggggggtccctgtgctatgaaggcacatagtcctagctctccacgtggcctccctccaagcagccttcagttctgggatggcaaataggccctcggagtagcttaatgctacttccccatccagggtctagagcagacatcactaatcgagccgtggcacttacagtccttcacagtctcagaatctgtagtcgtgatgtcaggcctgtgatttagaaactccaacctagctagtgcagctgaaggccacaggacaaagccaaaaccaaaagtcagttcttggctctcatagcagaagtccgggagacggaagccttcaggcatggctggatctgggcacctgcctgccctgcttggtctccaattggctttattcttgaccttttcacagtcagcaagatcaaagcccagctgtctcaggctcactagctggtagtgaagcctaaggctcctcccactggcagaggttcctctctatgcctgcacctggggtacatagggggaggggagggcgtaatgagctcccaccaccacccctagggagaggatcctcccaggggagaaggtctgaggaaaggactctgttgctttggatagtgatgttggcctagggccaggaggggtctctgtgtgtagtctgctgcctttgttacctgctacaaagactgggagggggaggggaggggatggaattcacctggtgagtgtcagaggtgtgtgtgtgtgtgtgtgtgtgtgtgtgtgtgtgtgtgttagcactacagtccccagagttgccagctggcaggttgacagctgagcaatctcagacaggtagagctgggccttgggaagaaccaggatacgaagagcagcctgtgggtaccggtagggacccggcaacgggcccaagtccacaggcagaataggtttagcaccttctacccacctgtcttagtcagggtttctattcctgcacaaacatcatgaccaagaagcaagttggggaggaaagggtttattcggcttacacttccatactgctgttcatcaccaaggaagtcaggactggaactcaagcaggtcaggaagcaggagctgatgcagaggccatggagggatgttctttactggcttgcctctcctggcttgctcagcctgctttcttatagaaccaagactaccagccctgagatggtcccacccacaaggggcctttcccccttgatcactaattgagaaaatgccttacagttggatctcatggaggcatttcctcaaccgaagctcctttctctgtgataactccagctgtgtcaagttgacacaaaagtagccagtacaccacccaagtgctgggataagctggtcccctcctgtggctccctcctgtggccagggcacatatgggctcagggaaagacagacctgtggataagacatgtagctgtgtcccaaccacctggacgccagatgcagttttgacagggaaaccagtgtcttgcacccagccatacagtgccaagtacgagcggctgcataggcccagactctcaagtcgccccacggcccctctctttcctctgtcacctcaccagcctgaggcacccccttctgctgatggagactgtttgtgggtggtggctggccggccgggttcaccctggggcctcacgattctctctgccctgggctttactggcctgtttctgctccacag

SEQ ID NO: 6-8 - skipped (3 entries)

SEQ ID NO: 9 - protein, 1391 aa, synthetic construct
MDKPKKKRKVKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 10 - DNA, 4176 bp, synthetic construct
  source 1..4176: mol_type = other DNA
atggacaagcccaagaaaaagcggaaagtgaagtacagcatcggcctggacatcggcaccaactctgtgggctgggccgtgatcaccgacgagtacaaggtgcccagcaagaaattcaaggtgctgggcaacaccgacaggcacagcatcaagaagaacctgatcggcgccctgctgttcgacagcggcgaaacagccgaggccaccagactgaagagaaccgccagaagaagatacaccaggcggaagaacaggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtga

SEQ ID NO: 11 - DNA, 4218 bp, synthetic construct
  source 1..4218: mol_type = other DNA
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtgagcggccgcttaatta

SEQ ID NO: 12 - protein, 1379 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 13 - RNA, 4514 nt, synthetic construct
  source 1..4514: mol_type = other RNA
gggtcccgcagtcggcgtccagcggctctgcttgttcgtgtgtgtgtcgttgcaggccttattcggatccatggataagaagtactcaatcgggctggatatcggaactaattccgtgggttgggcagtgatcacggatgaatacaaagtgccgtccaagaagttcaaggtcctggggaacaccgatagacacagcatcaagaaaaatctcatcggagccctgctgtttgactccggcgaaaccgcagaagcgacccggctcaaacgtaccgcgaggcgacgctacacccggcggaagaatcgcatctgctatctgcaagagatcttttcgaacgaaatggcaaaggtcgacgacagcttcttccaccgcctggaagaatctttcctggtggaggaggacaagaagcatgaacggcatcctatctttggaaacatcgtcgacgaagtggcgtaccacgaaaagtacccgaccatctaccatctgcggaagaagttggttgactcaactgacaaggccgacctcagattgatctacttggccctcgcccatatgatcaaattccgcggacacttcctgatcgaaggcgatctgaaccctgataactccgacgtggataagcttttcattcaactggtgcagacctacaaccaactgttcgaagaaaacccaatcaatgctagcggcgtcgatgccaaggccatcctgtccgcccggctgtcgaagtcgcggcgcctcgaaaacctgatcgcacagctgccgggagagaaaaagaacggacttttcggcaacttgatcgctctctcactgggactcactcccaatttcaagtccaattttgacctggccgaggacgcgaagctgcaactctcaaaggacacctacgacgacgacttggacaatttgctggcacaaattggcgatcagtacgcggatctgttccttgccgctaagaacctttcggacgcaatcttgctgtccgatatcctgcgcgtgaacaccgaaataaccaaagcgccgcttagcgcctcgatgattaagcggtacgacgagcatcaccaggatctcacgctgctcaaagcgctcgtgagacagcaactgcctgaaaagtacaaggagatcttcttcgaccagtccaagaatgggtacgcagggtacatcgatggaggcgctagccaggaagagttctataagttcatcaagccaatcctggaaaagatggacggaaccgaagaactgctggtcaagctgaacagggaggatctgctccggaaacagagaacctttgacaacggatccattccccaccagatccatctgggtgagctgcacgccatcttgcggcgccaggaggacttttacccattcctcaaggacaaccgggaaaagatcgagaaaattctgacgttccgcatcccgtattacgtgggcccactggcgcgcggcaattcgcgcttcgcgtggatgactagaaaatcagaggaaaccatcactccttggaatttcgaggaagttgtggataagggagcttcggcacaaagcttcatcgaacgaatgaccaacttcgacaagaatctcccaaacgagaaggtgcttcctaagcacagcctcctttacgaatacttcactgtctacaacgaactgactaaagtgaaatacgttactgaaggaatgaggaagccggcctttctgtccggagaacagaagaaagcaattgtcgatctgctgttcaagaccaaccgcaaggtgaccgtcaagcagcttaaagaggactacttcaagaagatcgagtgtttcgactcagtggaaatcagcggggtggaggacagattcaacgcttcgctgggaacctatcatgatctcctgaagatcatcaaggacaaggacttccttgacaacgaggagaacgaggacatcctggaagatatcgtcctgaccttgacccttttcgaggatcgcgagatgatcgaggagaggcttaagacctacgctcatctcttcgacgataaggtcatgaaacaactcaagcgccgccggtacactggttggggccgcctctcccgcaagctgatcaacggtattcgcgataaacagagcggtaaaactatcctggatttcctcaaatcggatggcttcgctaatcgtaacttcatgcaattgatccacgacgacagcctgacctttaaggaggacatccaaaaagcacaagtgtccggacagggagactcactccatgaacacatcgcgaatctggccggttcgccggcgattaagaagggaattctgcaaactgtgaaggtggtcgacgagctggtgaaggtcatgggacggcacaaaccggagaatatcgtgattgaaatggcccgagaaaaccagactacccagaagggccagaaaaactcccgcgaaaggatgaagcggatcgaagaaggaatcaaggagctgggcagccagatcctgaaagagcacccggtggaaaacacgcagctgcagaacgagaagctctacctgtactatttgcaaaatggacgggacatgtacgtggaccaagagctggacatcaatcggttgtctgattacgacgtggaccacatcgttccacagtcctttctgaaggatgactcgatcgataacaaggtgttgactcgcagcgacaagaacagagggaagtcagataatgtgccatcggaggaggtcgtgaagaagatgaagaattactggcggcagctcctgaatgcgaagctgattacccagagaaagtttgacaatctcactaaagccgagcgcggcggactctcagagctggataaggctggattcatcaaacggcagctggtcgagactcggcagattaccaagcacgtggcgcagatcttggactcccgcatgaacactaaatacgacgagaacgataagctcatccgggaagtgaaggtgattaccctgaaaagcaaacttgtgtcggactttcggaaggactttcagttttacaaagtgagagaaatcaacaactaccatcacgcgcatgacgcatacctcaacgctgtggtcggtaccgccctgatcaaaaagtaccctaaacttgaatcggagtttgtgtacggagactacaaggtctacgacgtgaggaagatgatagccaagtccgaacaggaaatcgggaaagcaactgcgaaatacttcttttactcaaacatcatgaactttttcaagactgaaattacgctggccaatggagaaatcaggaagaggccactgatcgaaactaacggagaaacgggcgaaatcgtgtgggacaagggcagggacttcgcaactgttcgcaaagtgctctctatgccgcaagtcaatattgtgaagaaaaccgaagtgcaaaccggcggattttcaaaggaatcgatcctcccaaagagaaatagcgacaagctcattgcacgcaagaaagactgggacccgaagaagtacggaggattcgattcgccgactgtcgcatactccgtcctcgtggtggccaaggtggagaagggaaagagcaaaaagctcaaatccgtcaaagagctgctggggattaccatcatggaacgatcctcgttcgagaagaacccgattgatttcctcgaggcgaagggttacaaggaggtgaagaaggatctgatcatcaaactccccaagtactcactgttcgaactggaaaatggtcggaagcgcatgctggcttcggccggagaactccaaaaaggaaatgagctggccttgcctagcaagtacgtcaacttcctctatcttgcttcgcactacgaaaaactcaaagggtcaccggaagataacgaacagaagcagcttttcgtggagcagcacaagcattatctggatgaaatcatcgaacaaatctccgagttttcaaagcgcgtgatcctcgccgacgccaacctcgacaaagtcctgtcggcctacaataagcatagagataagccgatcagagaacaggccgagaacattatccacttgttcaccctgactaacctgggagccccagccgccttcaagtacttcgatactactatcgatcgcaaaagatacacgtccaccaaggaagttctggacgcgaccctgatccaccaaagcatcactggactctacgaaactaggatcgatctgtcgcagctgggtggcgatggcggtggatctccgaaaaagaagagaaaggtgtaatgagctagccatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 14 - protein, 7 aa, synthetic construct
PKKKRKV

SEQ ID NO: 15 - protein, 7 aa, synthetic construct
PKKKRRV

SEQ ID NO: 16 - protein, 16 aa, synthetic construct
KRPAATKKAGQAKKKK

SEQ ID NO: 17 - RNA, 16 nt, synthetic construct
  source 1..16: mol_type = other RNA
gttttagagctatgct

SEQ ID NO: 18 - RNA, 22 nt, synthetic construct
  source 1..22: mol_type = other RNA
gttttagagctatgctgttttg

SEQ ID NO: 19 - RNA, 67 nt, synthetic construct
  source 1..67: mol_type = other RNA
agcatagcaagttaaaataaggctagtccgttatcaacttgaaaaagtggcaccgagtcggtgcttt

SEQ ID NO: 20 - RNA, 72 nt, synthetic construct
  source 1..72: mol_type = other RNA
aaacagcatagcaagttaaaataaggctagtccgttatcaacttgaaaaagtggcaccgagtcggtgctttt

SEQ ID NO: 21 - RNA, 82 nt, synthetic construct
  source 1..82: mol_type = other RNA
gttggaaccattcaaaacagcatagcaagttaaaataaggctagtccgttatcaacttgaaaaagtggcaccgagtcggtgc

SEQ ID NO: 22 - RNA, 77 nt, synthetic construct
  source 1..77: mol_type = other RNA
gttttagagctagaaatagcaagttaaaataaggctagtccgttatcaacttgaaaaagtggcaccgagtcggtgct

SEQ ID NO: 23 - RNA, 82 nt, synthetic construct; identical to SEQ ID NO: 21
  source 1..82: mol_type = other RNA

SEQ ID NO: 24 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 25 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 26 - RNA, 83 nt, synthetic construct
  source 1..83: mol_type = other RNA
gttttagagctagaaatagcaagttaaaataaggctagtccgttatcaacttgaaaaagtggcaccgagtcggtgcttttttt

SEQ ID NO: 27 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 28 - RNA, 92 nt, synthetic construct
  source 1..92: mol_type = other RNA
gtttaagagctatgctggaaacagcatagcaagtttaaataaggctagtccgttatcaacttgaaaaagtggcaccgagtcggtgctttttt

SEQ ID NO: 29 - RNA, 68 nt, synthetic construct
  source 1..68: mol_type = other RNA
gttttagagctagaaatagcaagttaaaataaggctagtccgttatcaacttggcaccgagtcggtgc

SEQ ID NO: 30 - RNA, 100 nt, synthetic construct
  source 1..100: mol_type = other RNA; note = n = a, c, g, or u
nnnnnnnnnnnnnnnnnnnngttttagagctagaaatagcaagttaaaataaggctagtccgttatcaacttgaaaaagtggcaccgagtcggtgctttt

SEQ ID NO: 31 - RNA, 20 nt, synthetic construct
  source 1..20: mol_type = other RNA
gagtgcgctttcagcagcgc

SEQ ID NO: 32 - RNA, 20 nt, synthetic construct
  source 1..20: mol_type = other RNA
cctctacactctaatttacg

SEQ ID NO: 33 - RNA, 20 nt, synthetic construct
  source 1..20: mol_type = other RNA
cagcgggggtggccccgtat

SEQ ID NO: 34 - RNA, 20 nt, synthetic construct
  source 1..20: mol_type = other RNA
agctagctgagacctatacg

SEQ ID NO: 35 - RNA, 20 nt, synthetic construct
  source 1..20: mol_type = other RNA
ccacgtaaattagagtgtag

SEQ ID NO: 36 - RNA, 20 nt, synthetic construct
  source 1..20: mol_type = other RNA
cactcgaatcttcaacactc

SEQ ID NO: 37 - RNA, 20 nt, synthetic construct
  source 1..20: mol_type = other RNA
actcgaatcttcaacactca

SEQ ID NO: 38 - RNA, 20 nt, synthetic construct
  source 1..20: mol_type = other RNA
gtttcacagaacggtgcatc

SEQ ID NO: 39 - RNA, 20 nt, synthetic construct
  source 1..20: mol_type = other RNA
ggttgccacaactaccaggt

SEQ ID NO: 40 - RNA, 20 nt, synthetic construct
  source 1..20: mol_type = other RNA
tggccttgttcccgtgtggc

SEQ ID NO: 41 - RNA, 20 nt, synthetic construct
  source 1..20: mol_type = other RNA
cggccagccacacgggaaca

SEQ ID NO: 42 - RNA, 20 nt, synthetic construct
  source 1..20: mol_type = other RNA
aaacaaggcatcacatagcg

SEQ ID NO: 43 - RNA, 20 nt, synthetic construct
  source 1..20: mol_type = other RNA
gtataggtctcagctagctg

SEQ ID NO: 44 - RNA, 20 nt, synthetic construct
  source 1..20: mol_type = other RNA
ggtgccttgattgtgaatct

SEQ ID NO: 45 - RNA, 20 nt, synthetic construct
  source 1..20: mol_type = other RNA
gtgcatcaggattgattagc

SEQ ID NO: 46 - RNA, 20 nt, synthetic construct
  source 1..20: mol_type = other RNA
ggggttcccaatgacttttc

SEQ ID NO: 47 - RNA, 20 nt, synthetic construct
  source 1..20: mol_type = other RNA
tcagctagctgagacctata

SEQ ID NO: 48 - RNA, 20 nt, synthetic construct
  source 1..20: mol_type = other RNA
gatgcaccgttctgtgaaac

SEQ ID NO: 49 - RNA, 20 nt, synthetic construct
  source 1..20: mol_type = other RNA
gacggttttgtccctgttgg

SEQ ID NO: 50 - RNA, 20 nt, synthetic construct
  source 1..20: mol_type = other RNA
cttgaaatgtagacccccag

SEQ ID NO: 51 - RNA, 20 nt, synthetic construct
  source 1..20: mol_type = other RNA
aagaagcaccaggtacagcg

SEQ ID NO: 52 - RNA, 20 nt, synthetic construct
  source 1..20: mol_type = other RNA
ttgccacaactaccaggtgg

SEQ ID NO: 53 - RNA, 20 nt, synthetic construct
  source 1..20: mol_type = other RNA
ttcttgaaatgtagaccccc

SEQ ID NO: 54 - RNA, 20 nt, synthetic construct
  source 1..20: mol_type = other RNA
agcaccaggtacagcggggg

SEQ ID NO: 55 - RNA, 20 nt, synthetic construct
  source 1..20: mol_type = other RNA
ttatgatgaacagtgtcatc

SEQ ID NO: 56 - RNA, 20 nt, synthetic construct
  source 1..20: mol_type = other RNA
catgttaaaatgaacacggg

SEQ ID NO: 57 - RNA, 20 nt, synthetic construct
  source 1..20: mol_type = other RNA
gaagagacactccacctgga

SEQ ID NO: 58 - RNA, 20 nt, synthetic construct
  source 1..20: mol_type = other RNA
tctgctctccagcactcggg

SEQ ID NO: 59 - RNA, 20 nt, synthetic construct
  source 1..20: mol_type = other RNA
gtctgagcagtagtccagga

SEQ ID NO: 60 - RNA, 20 nt, synthetic construct
  source 1..20: mol_type = other RNA
atctggcatacagagcccca

SEQ ID NO: 61 - RNA, 20 nt, synthetic construct
  source 1..20: mol_type = other RNA
cacaactaccaggtgggggg

SEQ ID NO: 62 - RNA, 20 nt, synthetic construct
  source 1..20: mol_type = other RNA
agagtaacagtgcagtgtgc

SEQ ID NO: 63 - RNA, 20 nt, synthetic construct
  source 1..20: mol_type = other RNA
tttaagagtcgagaaattct

SEQ ID NO: 64 - RNA, 20 nt, synthetic construct
  source 1..20: mol_type = other RNA
gttttgtccctgttgggggt

SEQ ID NO: 65 - RNA, 20 nt, synthetic construct
  source 1..20: mol_type = other RNA
gtggctggccgtcctgtgca

SEQ ID NO: 66 - RNA, 20 nt, synthetic construct
  source 1..20: mol_type = other RNA
catctggcatacagagcccc

SEQ ID NO: 67 - RNA, 20 nt, synthetic construct
  source 1..20: mol_type = other RNA
ctgtggccagtaggtgactt

SEQ ID NO: 68 - RNA, 20 nt, synthetic construct
  source 1..20: mol_type = other RNA
tcttgcatataacattagga

SEQ ID NO: 69 - RNA, 20 nt, synthetic construct
  source 1..20: mol_type = other RNA
atgaaggggcacaagtggca

SEQ ID NO: 70 - RNA, 20 nt, synthetic construct
  source 1..20: mol_type = other RNA
aaaagtcattgggaacccct

SEQ ID NO: 71 - RNA, 20 nt, synthetic construct
  source 1..20: mol_type = other RNA
gtggagggattaatgacaga

SEQ ID NO: 72 - RNA, 20 nt, synthetic construct
  source 1..20: mol_type = other RNA
ctgcagaagcaccccacaga

SEQ ID NO: 73 - RNA, 20 nt, synthetic construct
  source 1..20: mol_type = other RNA
aggcaaggacaggggccatc

SEQ ID NO: 74 - RNA, 20 nt, synthetic construct
  source 1..20: mol_type = other RNA
tctggcatacagagccccag

SEQ ID NO: 75 - RNA, 20 nt, synthetic construct
  source 1..20: mol_type = other RNA
gaagaagtaggcaaggacag

SEQ ID NO: 76 - RNA, 20 nt, synthetic construct
  source 1..20: mol_type = other RNA
agaaccattcctctggatcg

SEQ ID NO: 77 - RNA, 20 nt, synthetic construct
  source 1..20: mol_type = other RNA
cagctagctgagacctatac

SEQ ID NO: 78 - RNA, 20 nt, synthetic construct
  source 1..20: mol_type = other RNA
ggttgtctgagcagtagtcc

SEQ ID NO: 79 - RNA, 20 nt, synthetic construct
  source 1..20: mol_type = other RNA
gaaaccccgatccagaggaa

SEQ ID NO: 80 - RNA, 20 nt, synthetic construct
  source 1..20: mol_type = other RNA
tcacatagcgaggaagccct

SEQ ID NO: 81 - RNA, 20 nt, synthetic construct
  source 1..20: mol_type = other RNA
cagagaactccaattgagtc

SEQ ID NO: 82 - RNA, 20 nt, synthetic construct
  source 1..20: mol_type = other RNA
gggacaaaaccgtctctggt

SEQ ID NO: 83 - RNA, 20 nt, synthetic construct
  source 1..20: mol_type = other RNA
atcacatagcgaggaagccc

SEQ ID NO: 84 - RNA, 20 nt, synthetic construct
  source 1..20: mol_type = other RNA
ataaatgccctgtatgggac

SEQ ID NO: 85 - RNA, 20 nt, synthetic construct
  source 1..20: mol_type = other RNA
agacggttttgtccctgttg

SEQ ID NO: 86 - RNA, 20 nt, synthetic construct
  source 1..20: mol_type = other RNA
aaatgccctgtatgggacag

SEQ ID NO: 87 - RNA, 20 nt, synthetic construct
  source 1..20: mol_type = other RNA
tgattgtgaatctaggcatg

SEQ ID NO: 88 - RNA, 20 nt, synthetic construct
  source 1..20: mol_type = other RNA
gagacggttttgtccctgtt

SEQ ID NO: 89 - RNA, 20 nt, synthetic construct
  source 1..20: mol_type = other RNA
actgagaaaccccgatccag

SEQ ID NO: 90 - RNA, 20 nt, synthetic construct
  source 1..20: mol_type = other RNA
catagcgaggaagccctggg

SEQ ID NO: 91 - RNA, 20 nt, synthetic construct
  source 1..20: mol_type = other RNA
acattctagttccaggcctg

SEQ ID NO: 92 - RNA, 20 nt, synthetic construct
  source 1..20: mol_type = other RNA
actcgggaggcacagaacca

SEQ ID NO: 93 - RNA, 20 nt, synthetic construct
  source 1..20: mol_type = other RNA
catgcctagattcacaatca

SEQ ID NO: 94 - RNA, 20 nt, synthetic construct
  source 1..20: mol_type = other RNA
ggaactagaatgtccctcct

SEQ ID NO: 95 - RNA, 20 nt, synthetic construct
  source 1..20: mol_type = other RNA
ctcagctagctgaggcccct

SEQ ID NO: 96 - RNA, 20 nt, synthetic construct
  source 1..20: mol_type = other RNA
aagagacactccacctggat

SEQ ID NO: 97 - RNA, 20 nt, synthetic construct
  source 1..20: mol_type = other RNA
gaaaagtcattgggaacccc

SEQ ID NO: 98 - RNA, 20 nt, synthetic construct
  source 1..20: mol_type = other RNA
gccagtaggtgacttgggac

SEQ ID NO: 99 - RNA, 20 nt, synthetic construct
  source 1..20: mol_type = other RNA
atcaaggcaccagactcaat

SEQ ID NO: 100 - RNA, 20 nt, synthetic construct
  source 1..20: mol_type = other RNA
atcaggattgattagcaggc

SEQ ID NO: 101 - RNA, 20 nt, synthetic construct
  source 1..20: mol_type = other RNA
tgccacaactaccaggtggg

SEQ ID NO: 102 - RNA, 20 nt, synthetic construct
  source 1..20: mol_type = other RNA
gacactccacctggatggga

SEQ ID NO: 103 - RNA, 20 nt, synthetic construct
  source 1..20: mol_type = other RNA
gcagacccctgtcccataca

SEQ ID NO: 104 - RNA, 20 nt, synthetic construct
  source 1..20: mol_type = other RNA
gggatcagctctgaccctgc

SEQ ID NO: 105 - RNA, 20 nt, synthetic construct
  source 1..20: mol_type = other RNA
tcagctagctgaggcccctg

SEQ ID NO: 106 - RNA, 20 nt, synthetic construct
  source 1..20: mol_type = other RNA
ctggggctctgtatgccaga

SEQ ID NO: 107 - RNA, 20 nt, synthetic construct
  source 1..20: mol_type = other RNA
gagtaacagtgcagtgtgct

SEQ ID NO: 108 - RNA, 20 nt, synthetic construct
  source 1..20: mol_type = other RNA
gtgcttctgcagggttgcca

SEQ ID NO: 109 - RNA, 20 nt, synthetic construct
  source 1..20: mol_type = other RNA
tcagagctgatcccctctgt

SEQ ID NO: 110 - RNA, 20 nt, synthetic construct
  source 1..20: mol_type = other RNA
cgggaggcacagaaccaagg

SEQ ID NO: 111 - RNA, 20 nt, synthetic construct
  source 1..20: mol_type = other RNA
tgtctccctgaaaagtcatt

SEQ ID NO: 112 - RNA, 20 nt, synthetic construct
  source 1..20: mol_type = other RNA
aaagtcattgggaacccctg

SEQ ID NO: 113 - RNA, 20 nt, synthetic construct
  source 1..20: mol_type = other RNA
gtggtggatgctcctgcccc

SEQ ID NO: 114 - RNA, 20 nt, synthetic construct
  source 1..20: mol_type = other RNA
gtggaattctggcatctagt

SEQ ID NO: 115 - RNA, 20 nt, synthetic construct
  source 1..20: mol_type = other RNA
cctgtggccagtaggtgact

SEQ ID NO: 116 - RNA, 20 nt, synthetic construct
  source 1..20: mol_type = other RNA
atggatagaaaaacggtgaa

SEQ ID NO: 117 - RNA, 20 nt, synthetic construct
  source 1..20: mol_type = other RNA
ctccacctggatgggaaggt

SEQ ID NO: 118 - RNA, 20 nt, synthetic construct
  source 1..20: mol_type = other RNA
actccacctggatgggaagg

SEQ ID NO: 119 - DNA, 20 bp, synthetic construct
  source 1..20: mol_type = other DNA
gagtgcgctttcagcagcgc

SEQ ID NO: 120 - DNA, 20 bp, synthetic construct
  source 1..20: mol_type = other DNA
cctctacactctaatttacg

SEQ ID NO: 121 - DNA, 20 bp, synthetic construct
  source 1..20: mol_type = other DNA
cagcgggggtggccccgtat

SEQ ID NO: 122 - DNA, 20 bp, synthetic construct
  source 1..20: mol_type = other DNA
agctagctgagacctatacg

SEQ ID NO: 123 - DNA, 20 bp, synthetic construct
  source 1..20: mol_type = other DNA
ccacgtaaattagagtgtag

SEQ ID NO: 124 - DNA, 20 bp, synthetic construct
  source 1..20: mol_type = other DNA
cactcgaatcttcaacactc

SEQ ID NO: 125 - DNA, 20 bp, synthetic construct
  source 1..20: mol_type = other DNA
actcgaatcttcaacactca

SEQ ID NO: 126 - DNA, 20 bp, synthetic construct
  source 1..20: mol_type = other DNA
gtttcacagaacggtgcatc

SEQ ID NO: 127 - DNA, 20 bp, synthetic construct
  source 1..20: mol_type = other DNA
ggttgccacaactaccaggt

SEQ ID NO: 128 - DNA, 20 bp, synthetic construct
  source 1..20: mol_type = other DNA
tggccttgttcccgtgtggc

SEQ ID NO: 129 - DNA, 20 bp, synthetic construct
  source 1..20: mol_type = other DNA
cggccagccacacgggaaca

SEQ ID NO: 130 - DNA, 20 bp, synthetic construct
  source 1..20: mol_type = other DNA
aaacaaggcatcacatagcg

SEQ ID NO: 131 - DNA, 20 bp, synthetic construct
  source 1..20: mol_type = other DNA
gtataggtctcagctagctg

SEQ ID NO: 132 - DNA, 20 bp, synthetic construct
  source 1..20: mol_type = other DNA
ggtgccttgattgtgaatct

SEQ ID NO: 133 - DNA, 20 bp, synthetic construct
  source 1..20: mol_type = other DNA
gtgcatcaggattgattagc

SEQ ID NO: 134 - DNA, 20 bp, synthetic construct
  source 1..20: mol_type = other DNA
ggggttcccaatgacttttc

SEQ ID NO: 135 - DNA, 20 bp, synthetic construct
  source 1..20: mol_type = other DNA
tcagctagctgagacctata

SEQ ID NO: 136 - DNA, 20 bp, synthetic construct
  source 1..20: mol_type = other DNA
gatgcaccgttctgtgaaac

SEQ ID NO: 137 - DNA, 20 bp, synthetic construct
  source 1..20: mol_type = other DNA
gacggttttgtccctgttgg

SEQ ID NO: 138 - DNA, 20 bp, synthetic construct
  source 1..20: mol_type = other DNA
cttgaaatgtagacccccag

SEQ ID NO: 139 - DNA, 20 bp, synthetic construct
  source 1..20: mol_type = other DNA
aagaagcaccaggtacagcg

SEQ ID NO: 140 - DNA, 20 bp, synthetic construct
  source 1..20: mol_type = other DNA
ttgccacaactaccaggtgg

SEQ ID NO: 141 - DNA, 20 bp, synthetic construct
  source 1..20: mol_type = other DNA
ttcttgaaatgtagaccccc

SEQ ID NO: 142 - DNA, 20 bp, synthetic construct
  source 1..20: mol_type = other DNA
agcaccaggtacagcggggg

SEQ ID NO: 143 - DNA, 20 bp, synthetic construct
  source 1..20: mol_type = other DNA
ttatgatgaacagtgtcatc

SEQ ID NO: 144 - DNA, 20 bp, synthetic construct
  source 1..20: mol_type = other DNA
catgttaaaatgaacacggg

SEQ ID NO: 145 - DNA, 20 bp, synthetic construct
  source 1..20: mol_type = other DNA
gaagagacactccacctgga

SEQ ID NO: 146 - DNA, 20 bp, synthetic construct
  source 1..20: mol_type = other DNA
tctgctctccagcactcggg

SEQ ID NO: 147 - DNA, 20 bp, synthetic construct
  source 1..20: mol_type = other DNA
gtctgagcagtagtccagga

SEQ ID NO: 148 - DNA, 20 bp, synthetic construct
  source 1..20: mol_type = other DNA
atctggcatacagagcccca

SEQ ID NO: 149 - DNA, 20 bp, synthetic construct
  source 1..20: mol_type = other DNA
cacaactaccaggtgggggg

SEQ ID NO: 150 - DNA, 20 bp, synthetic construct
  source 1..20: mol_type = other DNA
agagtaacagtgcagtgtgc

SEQ ID NO: 151 - DNA, 20 bp, synthetic construct
  source 1..20: mol_type = other DNA
tttaagagtcgagaaattct

SEQ ID NO: 152 - DNA, 20 bp, synthetic construct
  source 1..20: mol_type = other DNA
gttttgtccctgttgggggt

SEQ ID NO: 153 - DNA, 20 bp, synthetic construct
  source 1..20: mol_type = other DNA
gtggctggccgtcctgtgca

SEQ ID NO: 154 - DNA, 20 bp, synthetic construct
  source 1..20: mol_type = other DNA
catctggcatacagagcccc

SEQ ID NO: 155 - DNA, 20 bp, synthetic construct
  source 1..20: mol_type = other DNA
ctgtggccagtaggtgactt

SEQ ID NO: 156 - DNA, 20 bp, synthetic construct
  source 1..20: mol_type = other DNA
tcttgcatataacattagga

SEQ ID NO: 157 - DNA, 20 bp, synthetic construct
  source 1..20: mol_type = other DNA
atgaaggggcacaagtggca

SEQ ID NO: 158 - DNA, 20 bp, synthetic construct
  source 1..20: mol_type = other DNA
aaaagtcattgggaacccct

SEQ ID NO: 159 - DNA, 20 bp, synthetic construct
  source 1..20: mol_type = other DNA
gtggagggattaatgacaga

SEQ ID NO: 160 - DNA, 20 bp, synthetic construct
  source 1..20: mol_type = other DNA
ctgcagaagcaccccacaga

SEQ ID NO: 161 - DNA, 20 bp, synthetic construct
  source 1..20: mol_type = other DNA
aggcaaggacaggggccatc

SEQ ID NO: 162 - DNA, 20 bp, synthetic construct
  source 1..20: mol_type = other DNA
tctggcatacagagccccag

SEQ ID NO: 163 - DNA, 20 bp, synthetic construct
  source 1..20: mol_type = other DNA
gaagaagtaggcaaggacag

SEQ ID NO: 164 - DNA, 20 bp, synthetic construct
  source 1..20: mol_type = other DNA
agaaccattcctctggatcg

SEQ ID NO: 165 - DNA, 20 bp, synthetic construct
  source 1..20: mol_type = other DNA
cagctagctgagacctatac

SEQ ID NO: 166 - DNA, 20 bp, synthetic construct
  source 1..20: mol_type = other DNA
ggttgtctgagcagtagtcc

SEQ ID NO: 167 - DNA, 20 bp, synthetic construct
  source 1..20: mol_type = other DNA
gaaaccccgatccagaggaa

SEQ ID NO: 168 - DNA, 20 bp, synthetic construct
  source 1..20: mol_type = other DNA
tcacatagcgaggaagccct

SEQ ID NO: 169 - DNA, 20 bp, synthetic construct
  source 1..20: mol_type = other DNA
cagagaactccaattgagtc

SEQ ID NO: 170 - DNA, 20 bp, synthetic construct
  source 1..20: mol_type = other DNA
gggacaaaaccgtctctggt

SEQ ID NO: 171 - DNA, 20 bp, synthetic construct
  source 1..20: mol_type = other DNA
atcacatagcgaggaagccc

SEQ ID NO: 172 - DNA, 20 bp, synthetic construct
  source 1..20: mol_type = other DNA
ataaatgccctgtatgggac

SEQ ID NO: 173 - DNA, 20 bp, synthetic construct
  source 1..20: mol_type = other DNA
agacggttttgtccctgttg

SEQ ID NO: 174 - DNA, 20 bp, synthetic construct
  source 1..20: mol_type = other DNA
aaatgccctgtatgggacag

SEQ ID NO: 175 - DNA, 20 bp, synthetic construct
  source 1..20: mol_type = other DNA
tgattgtgaatctaggcatg

SEQ ID NO: 176 - DNA, 20 bp, synthetic construct
  source 1..20: mol_type = other DNA
gagacggttttgtccctgtt

SEQ ID NO: 177 - DNA, 20 bp, synthetic construct
  source 1..20: mol_type = other DNA
actgagaaaccccgatccag

SEQ ID NO: 178 - DNA, 20 bp, synthetic construct
  source 1..20: mol_type = other DNA
catagcgaggaagccctggg

SEQ ID NO: 179 - DNA, 20 bp, synthetic construct
  source 1..20: mol_type = other DNA
acattctagttccaggcctg

SEQ ID NO: 180 - DNA, 20 bp, synthetic construct
  source 1..20: mol_type = other DNA
actcgggaggcacagaacca

SEQ ID NO: 181 - DNA, 20 bp, synthetic construct
  source 1..20: mol_type = other DNA
catgcctagattcacaatca

SEQ ID NO: 182 - DNA, 20 bp, synthetic construct
  source 1..20: mol_type = other DNA
ggaactagaatgtccctcct

SEQ ID NO: 183 - DNA, 20 bp, synthetic construct
  source 1..20: mol_type = other DNA
ctcagctagctgaggcccct

SEQ ID NO: 184 - DNA, 20 bp, synthetic construct
  source 1..20: mol_type = other DNA
aagagacactccacctggat

SEQ ID NO: 185 - DNA, 20 bp, synthetic construct
  source 1..20: mol_type = other DNA
gaaaagtcattgggaacccc

SEQ ID NO: 186 - DNA, 20 bp, synthetic construct
  source 1..20: mol_type = other DNA
gccagtaggtgacttgggac

SEQ ID NO: 187 - DNA, 20 bp, synthetic construct
  source 1..20: mol_type = other DNA
atcaaggcaccagactcaat

SEQ ID NO: 188 - DNA, 20 bp, synthetic construct
  source 1..20: mol_type = other DNA
atcaggattgattagcaggc

SEQ ID NO: 189 - DNA, 20 bp, synthetic construct
  source 1..20: mol_type = other DNA
tgccacaactaccaggtggg

SEQ ID NO: 190 - DNA, 20 bp, synthetic construct
  source 1..20: mol_type = other DNA
gacactccacctggatggga

SEQ ID NO: 191 - DNA, 20 bp, synthetic construct
  source 1..20: mol_type = other DNA
gcagacccctgtcccataca

SEQ ID NO: 192 - DNA, 20 bp, synthetic construct
  source 1..20: mol_type = other DNA
gggatcagctctgaccctgc

SEQ ID NO: 193 - DNA, 20 bp, synthetic construct
  source 1..20: mol_type = other DNA
tcagctagctgaggcccctg

SEQ ID NO: 194 - DNA, 20 bp, synthetic construct
  source 1..20: mol_type = other DNA
ctggggctctgtatgccaga

SEQ ID NO: 195 - DNA, 20 bp, synthetic construct
  source 1..20: mol_type = other DNA
gagtaacagtgcagtgtgct

SEQ ID NO: 196 - DNA, 20 bp, synthetic construct
  source 1..20: mol_type = other DNA
gtgcttctgcagggttgcca

SEQ ID NO: 197 - DNA, 20 bp, synthetic construct
  source 1..20: mol_type = other DNA
tcagagctgatcccctctgt

SEQ ID NO: 198 - DNA, 20 bp, synthetic construct
  source 1..20: mol_type = other DNA
cgggaggcacagaaccaagg

SEQ ID NO: 199 - DNA, 20 bp, synthetic construct
  source 1..20: mol_type = other DNA
tgtctccctgaaaagtcatt

SEQ ID NO: 200 - DNA, 20 bp, synthetic construct
  source 1..20: mol_type = other DNA
aaagtcattgggaacccctg

SEQ ID NO: 201 - DNA, 20 bp, synthetic construct
  source 1..20: mol_type = other DNA
gtggtggatgctcctgcccc

SEQ ID NO: 202 - DNA, 20 bp, synthetic construct
  source 1..20: mol_type = other DNA
gtggaattctggcatctagt

SEQ ID NO: 203 - DNA, 20 bp, synthetic construct
  source 1..20: mol_type = other DNA
cctgtggccagtaggtgact

SEQ ID NO: 204 - DNA, 20 bp, synthetic construct
  source 1..20: mol_type = other DNA
atggatagaaaaacggtgaa

SEQ ID NO: 205 - DNA, 20 bp, synthetic construct
  source 1..20: mol_type = other DNA
ctccacctggatgggaaggt

SEQ ID NO: 206 - DNA, 20 bp, synthetic construct
  source 1..20: mol_type = other DNA
actccacctggatgggaagg

SEQ ID NO: 207 - RNA, 20 nt, synthetic construct
  source 1..20: mol_type = other RNA
agacatcactaatcgagccg

SEQ ID NO: 208 - RNA, 20 nt, synthetic construct
  source 1..20: mol_type = other RNA
accggtagggacccggcaac

SEQ ID NO: 209 - RNA, 20 nt, synthetic construct
  source 1..20: mol_type = other RNA
tgtggacttgggcccgttgc

SEQ ID NO: 210 - RNA, 20 nt, synthetic construct
  source 1..20: mol_type = other RNA
cctatgcagccgctcgtact

SEQ ID NO: 211 - RNA, 20 nt, synthetic construct
  source 1..20: mol_type = other RNA
gaatctgtagtcgtgatgtc

SEQ ID NO: 212 - RNA, 20 nt, synthetic construct
  source 1..20: mol_type = other RNA
caaaactgcatctggcgtcc

SEQ ID NO: 213 - DNA, 20 bp, synthetic construct
  source 1..20: mol_type = other DNA
agacatcactaatcgagccg

SEQ ID NO: 214 - DNA, 20 bp, synthetic construct
  source 1..20: mol_type = other DNA
accggtagggacccggcaac

SEQ ID NO: 215 - DNA, 20 bp, synthetic construct
  source 1..20: mol_type = other DNA
tgtggacttgggcccgttgc

SEQ ID NO: 216 - DNA, 20 bp, synthetic construct
  source 1..20: mol_type = other DNA
cctatgcagccgctcgtact

SEQ ID NO: 217 - DNA, 20 bp, synthetic construct
  source 1..20: mol_type = other DNA
gaatctgtagtcgtgatgtc

SEQ ID NO: 218 - DNA, 20 bp, synthetic construct
  source 1..20: mol_type = other DNA
caaaactgcatctggcgtcc

SEQ ID NO: 219 - RNA, 100 nt, synthetic construct
  source 1..100: mol_type = other RNA
agacatcactaatcgagccggttttagagctagaaatagcaagttaaaataaggctagtccgttatcaacttgaaaaagtggcaccgagtcggtgctttt

SEQ ID NO: 220 - RNA, 100 nt, synthetic construct
  source 1..100: mol_type = other RNA
accggtagggacccggcaacgttttagagctagaaatagcaagttaaaataaggctagtccgttatcaacttgaaaaagtggcaccgagtcggtgctttt

SEQ ID NO: 221 - RNA, 100 nt, synthetic construct
  source 1..100: mol_type = other RNA
tgtggacttgggcccgttgcgttttagagctagaaatagcaagttaaaataaggctagtccgttatcaacttgaaaaagtggcaccgagtcggtgctttt

SEQ ID NO: 222 - RNA, 100 nt, synthetic construct
  source 1..100: mol_type = other RNA
cctatgcagccgctcgtactgttttagagctagaaatagcaagttaaaataaggctagtccgttatcaacttgaaaaagtggcaccgagtcggtgctttt

SEQ ID NO: 223 - RNA, 100 nt, synthetic construct
  source 1..100: mol_type = other RNA
gaatctgtagtcgtgatgtcgttttagagctagaaatagcaagttaaaataaggctagtccgttatcaacttgaaaaagtggcaccgagtcggtgctttt

SEQ ID NO: 224 - RNA, 100 nt, synthetic construct
  source 1..100: mol_type = other RNA
caaaactgcatctggcgtccgttttagagctagaaatagcaagttaaaataaggctagtccgttatcaacttgaaaaagtggcaccgagtcggtgctttt

SEQ ID NO: 225 - DNA, 1134 bp, synthetic construct
  source 1..1134: mol_type = other DNA
gccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatag

SEQ ID NO: 226 - DNA, 1134 bp, synthetic construct
  source 1..1134: mol_type = other DNA
gccaacatcggacaaaaggaggatttcgaggaagccagaaagaaagccctgaagctgggcgccaagaaagtgttcattgaagatgtgtctcgggaattcgtggaagaatttatctggcctgctatccagagcagcgccctgtacgaggaccggtacctgctgggcaccagcctggcccgcccctgcatcgccagaaagcaagtggagatcgcccagagagagggcgccaaatacgtgagccacggcgctacaggcaaaggcaacgatcaggtcagattcgagctgagctgctacagcctggcccctcagatcaaggtgatcgctccttggcggatgccagaattctacaaccgcttcaagggcaggaacgacctgatggaatacgccaagcagcacggaatccccatccctgtgacccccaagaacccctggagcatggatgagaatctgatgcacatcagctacgaagctggcatcctggaaaaccctaagaaccaggccccacctggcctgtataccaagacccaggaccccgccaaggcccctaacaccccagacatcctggaaatcgagtttaagaagggagtgcccgtgaaggtcacaaacgtgaaggacggcactacccaccagaccagcctggaactgttcatgtacctgaacgaggtggctggcaagcacggcgtgggaagaatcgacatcgtggaaaatagatttatcggcatgaaaagcagaggcatctacgaaacccctgccggaaccatcctgtaccacgcccacctggatatcgaggccttcacaatggatagagaggtgcggaagatcaagcaaggcctgggcctgaaattcgccgagctggtgtacaccggattctggcattctccagagtgcgagttcgtccggcactgtattgctaagtcccaggagagagtggaaggcaaagtgcaggtgagcgtgctgaagggacaggtgtacatcctgggcagagagtctcctctgtccctctataatgaggagctggtgtctatgaatgttcagggcgactacgagcctaccgacgccacaggcttcatcaacatcaacagccttagactgaaggagtaccacagactgcagtccaaggtgacagccaagtga

SEQ ID NO: 227 - protein, 377 aa, synthetic construct
ANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAK

SEQ ID NO: 228 - DNA, 1062 bp, synthetic construct
  source 1..1062: mol_type = other DNA
gtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaa

SEQ ID NO: 229 - DNA, 1062 bp, synthetic construct
  source 1..1062: mol_type = other DNA
gtgttcattgaagatgtgtctcgggaattcgtggaagaatttatctggcctgctatccagagcagcgccctgtacgaggaccggtacctgctgggcaccagcctggcccgcccctgcatcgccagaaagcaagtggagatcgcccagagagagggcgccaaatacgtgagccacggcgctacaggcaaaggcaacgatcaggtcagattcgagctgagctgctacagcctggcccctcagatcaaggtgatcgctccttggcggatgccagaattctacaaccgcttcaagggcaggaacgacctgatggaatacgccaagcagcacggaatccccatccctgtgacccccaagaacccctggagcatggatgagaatctgatgcacatcagctacgaagctggcatcctggaaaaccctaagaaccaggccccacctggcctgtataccaagacccaggaccccgccaaggcccctaacaccccagacatcctggaaatcgagtttaagaagggagtgcccgtgaaggtcacaaacgtgaaggacggcactacccaccagaccagcctggaactgttcatgtacctgaacgaggtggctggcaagcacggcgtgggaagaatcgacatcgtggaaaatagatttatcggcatgaaaagcagaggcatctacgaaacccctgccggaaccatcctgtaccacgcccacctggatatcgaggccttcacaatggatagagaggtgcggaagatcaagcaaggcctgggcctgaaattcgccgagctggtgtacaccggattctggcattctccagagtgcgagttcgtccggcactgtattgctaagtcccaggagagagtggaaggcaaagtgcaggtgagcgtgctgaagggacaggtgtacatcctgggcagagagtctcctctgtccctctataatgaggagctggtgtctatgaatgttcagggcgactacgagcctaccgacgccacaggcttcatcaacatcaacagccttagactgaaggagtaccacagactgcagtccaaggtgacagccaag

SEQ ID NO: 230 - protein, 354 aa, synthetic construct [sequence omitted]

SEQ ID NO: 231 - DNA, 2718 bp, synthetic construct
  source 1..2718: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatatgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatagacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 232 - DNA, 3245 bp, synthetic construct
  source 1..3245: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctaggtcagtgaagagaagaacaaaaagcagcatattacagttagttgtcttcatcaatctttaaatatgttgtgtggtttttctctccctgtttccacaggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccggatccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgactagtcacttggctgtcaccttggactgcagtctgtggtactccttcagtctaaggctgttgatgttgatgaagcctgtggcgtcggtaggctcgtagtcgccctgaacattcatagacaccagctcctcattatagagggacagaggagactctctgcccaggatgtacacctgtcccttcagcacgctcacctgcactttgccttccactctctcctgggacttagcaatacagtgccggacgaactcgcactctggagaatgccagaatccggtgtacaccagctcggcgaatttcaggcccaggccttgcttgatcttccgcacctctctatccattgtgaaggcctcgatatccaggtgggcgtggtacaggatggttccggcaggggtttcgtagatgcctctgcttttcatgccgataaatctattttccacgatgtcgattcttcccacgccgtgcttgccagccacctcgttcaggtacatgaacagttccaggctggtctggtgggtagtgccgtccttcacgtttgtgaccttcacgggcactcccttcttaaactcgatttccaggatgtctggggtgttaggggccttggcggggtcctgggtcttggtatacaggccaggtggggcctggttcttagggttttccaggatgccagcttcgtagctgatgtgcatcagattctcatccatgctccaggggttcttgggggtcacagggatggggattccgtgctgcttggcgtattccatcaggtcgttcctgcccttgaagcggttgtagaattctggcatccgccaaggagcgatcaccttgatctgaggggccaggctgtagcagctcagctcgaatctgacctgatcgttgcctttgcctgtagcgccgtggctcacgtatttggcgccctctctctgggcgatctccacttgctttctggcgatgcaggggcgggccaggctggtgcccagcaggtaccggtcctcgtacagggcgctgctctggatagcaggccagataaattcttccacgaattcccgagacacatcttcaatgaacactttcttggcgcccagcttcagggctttctttctggcttcctcgaaatcctccttttgtccgatgttggcctgtggaaacagggagagaaaaaccacacaacatatttaaagattgatgaagacaactaactgtaatatgctgctttttgttcttctcttcactgacctagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 233 - DNA, 3203 bp, synthetic construct
  source 1..3203: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctaggtcagtgaagagaagaacaaaaagcagcatattacagttagttgtcttcatcaatctttaaatatgttgtgtggtttttctctccctgtttccacaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaaggcggaggtgggtccgtgagcggctggcggctgttcaagaagattagctagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccggatccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgactagtcagctgatcttcttgaacagtctccagccagacacggacccacctccgcccttggctgtcaccttggactgcagtctgtggtactccttcagtctaaggctgttgatgttgatgaagcctgtggcgtcggtaggctcgtagtcgccctgaacattcatagacaccagctcctcattatagagggacagaggagactctctgcccaggatgtacacctgtcccttcagcacgctcacctgcactttgccttccactctctcctgggacttagcaatacagtgccggacgaactcgcactctggagaatgccagaatccggtgtacaccagctcggcgaatttcaggcccaggccttgcttgatcttccgcacctctctatccattgtgaaggcctcgatatccaggtgggcgtggtacaggatggttccggcaggggtttcgtagatgcctctgcttttcatgccgataaatctattttccacgatgtcgattcttcccacgccgtgcttgccagccacctcgttcaggtacatgaacagttccaggctggtctggtgggtagtgccgtccttcacgtttgtgaccttcacgggcactcccttcttaaactcgatttccaggatgtctggggtgttaggggccttggcggggtcctgggtcttggtatacaggccaggtggggcctggttcttagggttttccaggatgccagcttcgtagctgatgtgcatcagattctcatccatgctccaggggttcttgggggtcacagggatggggattccgtgctgcttggcgtattccatcaggtcgttcctgcccttgaagcggttgtagaattctggcatccgccaaggagcgatcaccttgatctgaggggccaggctgtagcagctcagctcgaatctgacctgatcgttgcctttgcctgtagcgccgtggctcacgtatttggcgccctctctctgggcgatctccacttgctttctggcgatgcaggggcgggccaggctggtgcccagcaggtaccggtcctcgtacagggcgctgctctggatagcaggccagataaattcttccacgaattcccgagacacatcttcaatgaacacctgtggaaacagggagagaaaaaccacacaacatatttaaagattgatgaagacaactaactgtaatatgctgctttttgttcttctcttcactgacctagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 234 - DNA, 321 bp, synthetic construct
  source 1..321: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 235 - DNA, 479 bp, synthetic construct
  source 1..479: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 236 - DNA, 276 bp, synthetic construct
  source 1..276: mol_type = other DNA
cctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatcccc

SEQ ID NO: 237 - DNA, 276 bp, synthetic construct
  source 1..276: mol_type = other DNA
cctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatcccc

SEQ ID NO: 238 - DNA, 224 bp, synthetic construct
  source 1..224: mol_type = other DNA
ctgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatg

SEQ ID NO: 239 - DNA, 192 bp, synthetic construct
  source 1..192: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttt

SEQ ID NO: 240 - DNA, 131 bp, synthetic construct
  source 1..131: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 241 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 242 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 243 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 244 - DNA, 397 bp, synthetic construct
  source 1..397: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 245 - DNA, 33 bp, synthetic construct
  source 1..33: mol_type = other DNA
gtgagcggctggcggctgttcaagaagattagc

SEQ ID NO: 246 - DNA, 33 bp, synthetic construct
  source 1..33: mol_type = other DNA
gtgtctggctggagactgttcaagaagatcagc

SEQ ID NO: 247 - protein, 11 aa, synthetic construct
VSGWRLFKKIS

SEQ ID NO: 248 - DNA, 100 bp, synthetic construct
  source 1..100: mol_type = other DNA
taggtcagtgaagagaagaacaaaaagcagcatattacagttagttgtcttcatcaatctttaaatatgttgtgtggtttttctctccctgtttccacag

SEQ ID NO: 249 - RNA, 4423 nt, synthetic construct
  source 1..4423: mol_type = other RNA
gggaagctcagaataaacgctcaactttggccggatctgccaccatggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 250 - RNA, 4140 nt, synthetic construct
  source 1..4140: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 251 - DNA, 4140 bp, synthetic construct
  source 1..4140: mol_type = other DNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 252 - RNA, 100 nt, synthetic construct
  source 1..100: mol_type = other RNA
gagtgcgctttcagcagcgcgttttagagctagaaatagcaagttaaaataaggctagtccgttatcaacttgaaaaagtggcaccgagtcggtgctttt

SEQ ID NO: 253 - RNA, 100 nt, synthetic construct
  source 1..100: mol_type = other RNA
cctctacactctaatttacggttttagagctagaaatagcaagttaaaataaggctagtccgttatcaacttgaaaaagtggcaccgagtcggtgctttt

SEQ ID NO: 254 - RNA, 100 nt, synthetic construct
  source 1..100: mol_type = other RNA
cagcgggggtggccccgtatgttttagagctagaaatagcaagttaaaataaggctagtccgttatcaacttgaaaaagtggcaccgagtcggtgctttt

SEQ ID NO: 255 - RNA, 100 nt, synthetic construct
  source 1..100: mol_type = other RNA
agctagctgagacctatacggttttagagctagaaatagcaagttaaaataaggctagtccgttatcaacttgaaaaagtggcaccgagtcggtgctttt

SEQ ID NO: 256 - RNA, 100 nt, synthetic construct
  source 1..100: mol_type = other RNA
ccacgtaaattagagtgtaggttttagagctagaaatagcaagttaaaataaggctagtccgttatcaacttgaaaaagtggcaccgagtcggtgctttt

SEQ ID NO: 257 - RNA, 100 nt, synthetic construct
  source 1..100: mol_type = other RNA
cactcgaatcttcaacactcgttttagagctagaaatagcaagttaaaataaggctagtccgttatcaacttgaaaaagtggcaccgagtcggtgctttt

SEQ ID NO: 258 - RNA, 100 nt, synthetic construct
  source 1..100: mol_type = other RNA
actcgaatcttcaacactcagttttagagctagaaatagcaagttaaaataaggctagtccgttatcaacttgaaaaagtggcaccgagtcggtgctttt

SEQ ID NO: 259 - RNA, 100 nt, synthetic construct
  source 1..100: mol_type = other RNA
gtttcacagaacggtgcatcgttttagagctagaaatagcaagttaaaataaggctagtccgttatcaacttgaaaaagtggcaccgagtcggtgctttt

SEQ ID NO: 260 - RNA, 100 nt, synthetic construct
  source 1..100: mol_type = other RNA
ggttgccacaactaccaggtgttttagagctagaaatagcaagttaaaataaggctagtccgttatcaacttgaaaaagtggcaccgagtcggtgctttt

SEQ ID NO: 261 - RNA, 100 nt, synthetic construct
  source 1..100: mol_type = other RNA
tggccttgttcccgtgtggcgttttagagctagaaatagcaagttaaaataaggctagtccgttatcaacttgaaaaagtggcaccgagtcggtgctttt

SEQ ID NO: 262 - RNA, 100 nt, synthetic construct
  source 1..100: mol_type = other RNA
cggccagccacacgggaacagttttagagctagaaatagcaagttaaaataaggctagtccgttatcaacttgaaaaagtggcaccgagtcggtgctttt

SEQ ID NO: 263 - RNA, 100 nt, synthetic construct
  source 1..100: mol_type = other RNA
aaacaaggcatcacatagcggttttagagctagaaatagcaagttaaaataaggctagtccgttatcaacttgaaaaagtggcaccgagtcggtgctttt

SEQ ID NO: 264 - RNA, 100 nt, synthetic construct
  source 1..100: mol_type = other RNA
gtataggtctcagctagctggttttagagctagaaatagcaagttaaaataaggctagtccgttatcaacttgaaaaagtggcaccgagtcggtgctttt

SEQ ID NO: 265 - RNA, 100 nt, synthetic construct
  source 1..100: mol_type = other RNA
ggtgccttgattgtgaatctgttttagagctagaaatagcaagttaaaataaggctagtccgttatcaacttgaaaaagtggcaccgagtcggtgctttt

SEQ ID NO: 266 - RNA, 100 nt, synthetic construct
  source 1..100: mol_type = other RNA
gtgcatcaggattgattagcgttttagagctagaaatagcaagttaaaataaggctagtccgttatcaacttgaaaaagtggcaccgagtcggtgctttt

SEQ ID NO: 267 - RNA, 100 nt, synthetic construct
  source 1..100: mol_type = other RNA
ggggttcccaatgacttttcgttttagagctagaaatagcaagttaaaataaggctagtccgttatcaacttgaaaaagtggcaccgagtcggtgctttt

SEQ ID NO: 268 - RNA, 100 nt, synthetic construct
  source 1..100: mol_type = other RNA
tcagctagctgagacctatagttttagagctagaaatagcaagttaaaataaggctagtccgttatcaacttgaaaaagtggcaccgagtcggtgctttt

SEQ ID NO: 269 - RNA, 100 nt, synthetic construct
  source 1..100: mol_type = other RNA
gatgcaccgttctgtgaaacgttttagagctagaaatagcaagttaaaataaggctagtccgttatcaacttgaaaaagtggcaccgagtcggtgctttt

SEQ ID NO: 270 - RNA, 100 nt, synthetic construct
  source 1..100: mol_type = other RNA
gacggttttgtccctgttgggttttagagctagaaatagcaagttaaaataaggctagtccgttatcaacttgaaaaagtggcaccgagtcggtgctttt

SEQ ID NO: 271 - RNA, 100 nt, synthetic construct
  source 1..100: mol_type = other RNA
cttgaaatgtagacccccaggttttagagctagaaatagcaagttaaaataaggctagtccgttatcaacttgaaaaagtggcaccgagtcggtgctttt

SEQ ID NO: 272 - RNA, 100 nt, synthetic construct
  source 1..100: mol_type = other RNA
aagaagcaccaggtacagcggttttagagctagaaatagcaagttaaaataaggctagtccgttatcaacttgaaaaagtggcaccgagtcggtgctttt

SEQ ID NO: 273 - RNA, 100 nt, synthetic construct
  source 1..100: mol_type = other RNA
ttgccacaactaccaggtgggttttagagctagaaatagcaagttaaaataaggctagtccgttatcaacttgaaaaagtggcaccgagtcggtgctttt

SEQ ID NO: 274 - RNA, 100 nt, synthetic construct
  source 1..100: mol_type = other RNA
ttcttgaaatgtagacccccgttttagagctagaaatagcaagttaaaataaggctagtccgttatcaacttgaaaaagtggcaccgagtcggtgctttt

SEQ ID NO: 275 - RNA, 100 nt, synthetic construct
  source 1..100: mol_type = other RNA
agcaccaggtacagcggggggttttagagctagaaatagcaagttaaaataaggctagtccgttatcaacttgaaaaagtggcaccgagtcggtgctttt

SEQ ID NO: 276 - RNA, 100 nt, synthetic construct
  source 1..100: mol_type = other RNA
ttatgatgaacagtgtcatcgttttagagctagaaatagcaagttaaaataaggctagtccgttatcaacttgaaaaagtggcaccgagtcggtgctttt

SEQ ID NO: 277 - RNA, 100 nt, synthetic construct
  source 1..100: mol_type = other RNA
catgttaaaatgaacacggggttttagagctagaaatagcaagttaaaataaggctagtccgttatcaacttgaaaaagtggcaccgagtcggtgctttt

SEQ ID NO: 278 - RNA, 100 nt, synthetic construct
  source 1..100: mol_type = other RNA
gaagagacactccacctggagttttagagctagaaatagcaagttaaaataaggctagtccgttatcaacttgaaaaagtggcaccgagtcggtgctttt

SEQ ID NO: 279 - RNA, 100 nt, synthetic construct
  source 1..100: mol_type = other RNA
tctgctctccagcactcggggttttagagctagaaatagcaagttaaaataaggctagtccgttatcaacttgaaaaagtggcaccgagtcggtgctttt

SEQ ID NO: 280 - RNA, 100 nt, synthetic construct
  source 1..100: mol_type = other RNA
gtctgagcagtagtccaggagttttagagctagaaatagcaagttaaaataaggctagtccgttatcaacttgaaaaagtggcaccgagtcggtgctttt

SEQ ID NO: 281 - RNA, 100 nt, synthetic construct
  source 1..100: mol_type = other RNA
atctggcatacagagccccagttttagagctagaaatagcaagttaaaataaggctagtccgttatcaacttgaaaaagtggcaccgagtcggtgctttt

SEQ ID NO: 282 - RNA, 100 nt, synthetic construct
  source 1..100: mol_type = other RNA
cacaactaccaggtgggggggttttagagctagaaatagcaagttaaaataaggctagtccgttatcaacttgaaaaagtggcaccgagtcggtgctttt

SEQ ID NO: 283 - RNA, 100 nt, synthetic construct
  source 1..100: mol_type = other RNA
agagtaacagtgcagtgtgcgttttagagctagaaatagcaagttaaaataaggctagtccgttatcaacttgaaaaagtggcaccgagtcggtgctttt

SEQ ID NO: 284 - RNA, 100 nt, synthetic construct
  source 1..100: mol_type = other RNA
tttaagagtcgagaaattctgttttagagctagaaatagcaagttaaaataaggctagtccgttatcaacttgaaaaagtggcaccgagtcggtgctttt

SEQ ID NO: 285 - RNA, 100 nt, synthetic construct
  source 1..100: mol_type = other RNA
gttttgtccctgttgggggtgttttagagctagaaatagcaagttaaaataaggctagtccgttatcaacttgaaaaagtggcaccgagtcggtgctttt

SEQ ID NO: 286 - RNA, 100 nt, synthetic construct
  source 1..100: mol_type = other RNA
gtggctggccgtcctgtgcagttttagagctagaaatagcaagttaaaataaggctagtccgttatcaacttgaaaaagtggcaccgagtcggtgctttt

SEQ ID NO: 287 - RNA, 100 nt, synthetic construct
  source 1..100: mol_type = other RNA
catctggcatacagagccccgttttagagctagaaatagcaagttaaaataaggctagtccgttatcaacttgaaaaagtggcaccgagtcggtgctttt

SEQ ID NO: 288 - RNA, 100 nt, synthetic construct
  source 1..100: mol_type = other RNA
ctgtggccagtaggtgacttgttttagagctagaaatagcaagttaaaataaggctagtccgttatcaacttgaaaaagtggcaccgagtcggtgctttt

SEQ ID NO: 289 - RNA, 100 nt, synthetic construct
  source 1..100: mol_type = other RNA
tcttgcatataacattaggagttttagagctagaaatagcaagttaaaataaggctagtccgttatcaacttgaaaaagtggcaccgagtcggtgctttt

SEQ ID NO: 290 - RNA, 100 nt, synthetic construct
  source 1..100: mol_type = other RNA
atgaaggggcacaagtggcagttttagagctagaaatagcaagttaaaataaggctagtccgttatcaacttgaaaaagtggcaccgagtcggtgctttt

SEQ ID NO: 291 - RNA, 100 nt, synthetic construct
  source 1..100: mol_type = other RNA
aaaagtcattgggaacccctgttttagagctagaaatagcaagttaaaataaggctagtccgttatcaacttgaaaaagtggcaccgagtcggtgctttt

SEQ ID NO: 292 - RNA, 100 nt, synthetic construct
  source 1..100: mol_type = other RNA
gtggagggattaatgacagagttttagagctagaaatagcaagttaaaataaggctagtccgttatcaacttgaaaaagtggcaccgagtcggtgctttt

SEQ ID NO: 293 - RNA, 100 nt, synthetic construct
  source 1..100: mol_type = other RNA
ctgcagaagcaccccacagagttttagagctagaaatagcaagttaaaataaggctagtccgttatcaacttgaaaaagtggcaccgagtcggtgctttt

SEQ ID NO: 294 - RNA, 100 nt, synthetic construct
  source 1..100: mol_type = other RNA
aggcaaggacaggggccatcgttttagagctagaaatagcaagttaaaataaggctagtccgttatcaacttgaaaaagtggcaccgagtcggtgctttt

SEQ ID NO: 295 - RNA, 100 nt, synthetic construct
  source 1..100: mol_type = other RNA
tctggcatacagagccccaggttttagagctagaaatagcaagttaaaataaggctagtccgttatcaacttgaaaaagtggcaccgagtcggtgctttt

SEQ ID NO: 296 - RNA, 100 nt, synthetic construct
  source 1..100: mol_type = other RNA
gaagaagtaggcaaggacaggttttagagctagaaatagcaagttaaaataaggctagtccgttatcaacttgaaaaagtggcaccgagtcggtgctttt

SEQ ID NO: 297 - RNA, 100 nt, synthetic construct
  source 1..100: mol_type = other RNA
agaaccattcctctggatcggttttagagctagaaatagcaagttaaaataaggctagtccgttatcaacttgaaaaagtggcaccgagtcggtgctttt

SEQ ID NO: 298 - RNA, 100 nt, synthetic construct
  source 1..100: mol_type = other RNA
cagctagctgagacctatacgttttagagctagaaatagcaagttaaaataaggctagtccgttatcaacttgaaaaagtggcaccgagtcggtgctttt

SEQ ID NO: 299 - RNA, 100 nt, synthetic construct
  source 1..100: mol_type = other RNA
ggttgtctgagcagtagtccgttttagagctagaaatagcaagttaaaataaggctagtccgttatcaacttgaaaaagtggcaccgagtcggtgctttt

SEQ ID NO: 300 - RNA, 100 nt, synthetic construct
  source 1..100: mol_type = other RNA
gaaaccccgatccagaggaagttttagagctagaaatagcaagttaaaataaggctagtccgttatcaacttgaaaaagtggcaccgagtcggtgctttt

SEQ ID NO: 301 - RNA, 100 nt, synthetic construct
  source 1..100: mol_type = other RNA
tcacatagcgaggaagccctgttttagagctagaaatagcaagttaaaataaggctagtccgttatcaacttgaaaaagtggcaccgagtcggtgctttt

SEQ ID NO: 302 - RNA, 100 nt, synthetic construct
  source 1..100: mol_type = other RNA
cagagaactccaattgagtcgttttagagctagaaatagcaagttaaaataaggctagtccgttatcaacttgaaaaagtggcaccgagtcggtgctttt

SEQ ID NO: 303 - RNA, 100 nt, synthetic construct
  source 1..100: mol_type = other RNA
gggacaaaaccgtctctggtgttttagagctagaaatagcaagttaaaataaggctagtccgttatcaacttgaaaaagtggcaccgagtcggtgctttt

SEQ ID NO: 304 - RNA, 100 nt, synthetic construct
  source 1..100: mol_type = other RNA
atcacatagcgaggaagcccgttttagagctagaaatagcaagttaaaataaggctagtccgttatcaacttgaaaaagtggcaccgagtcggtgctttt

SEQ ID NO: 305 - RNA, 100 nt, synthetic construct
  source 1..100: mol_type = other RNA
ataaatgccctgtatgggacgttttagagctagaaatagcaagttaaaataaggctagtccgttatcaacttgaaaaagtggcaccgagtcggtgctttt

SEQ ID NO: 306 - RNA, 100 nt, synthetic construct
  source 1..100: mol_type = other RNA
agacggttttgtccctgttggttttagagctagaaatagcaagttaaaataaggctagtccgttatcaacttgaaaaagtggcaccgagtcggtgctttt

SEQ ID NO: 307 - RNA, 100 nt, synthetic construct
  source 1..100: mol_type = other RNA
aaatgccctgtatgggacaggttttagagctagaaatagcaagttaaaataaggctagtccgttatcaacttgaaaaagtggcaccgagtcggtgctttt

SEQ ID NO: 308 - RNA, 100 nt, synthetic construct
  source 1..100: mol_type = other RNA
tgattgtgaatctaggcatggttttagagctagaaatagcaagttaaaataaggctagtccgttatcaacttgaaaaagtggcaccgagtcggtgctttt

SEQ ID NO: 309 - RNA, 100 nt, synthetic construct
  source 1..100: mol_type = other RNA
gagacggttttgtccctgttgttttagagctagaaatagcaagttaaaataaggctagtccgttatcaacttgaaaaagtggcaccgagtcggtgctttt

SEQ ID NO: 310 - RNA, 100 nt, synthetic construct
  source 1..100: mol_type = other RNA
actgagaaaccccgatccaggttttagagctagaaatagcaagttaaaataaggctagtccgttatcaacttgaaaaagtggcaccgagtcggtgctttt

SEQ ID NO: 311 - RNA, 100 nt, synthetic construct
  source 1..100: mol_type = other RNA
catagcgaggaagccctggggttttagagctagaaatagcaagttaaaataaggctagtccgttatcaacttgaaaaagtggcaccgagtcggtgctttt

SEQ ID NO: 312 - RNA, 100 nt, synthetic construct
  source 1..100: mol_type = other RNA
acattctagttccaggcctggttttagagctagaaatagcaagttaaaataaggctagtccgttatcaacttgaaaaagtggcaccgagtcggtgctttt

SEQ ID NO: 313 - RNA, 100 nt, synthetic construct
  source 1..100: mol_type = other RNA
actcgggaggcacagaaccagttttagagctagaaatagcaagttaaaataaggctagtccgttatcaacttgaaaaagtggcaccgagtcggtgctttt

SEQ ID NO: 314 - RNA, 100 nt, synthetic construct
  source 1..100: mol_type = other RNA
catgcctagattcacaatcagttttagagctagaaatagcaagttaaaataaggctagtccgttatcaacttgaaaaagtggcaccgagtcggtgctttt

SEQ ID NO: 315 - RNA, 100 nt, synthetic construct
  source 1..100: mol_type = other RNA
ggaactagaatgtccctcctgttttagagctagaaatagcaagttaaaataaggctagtccgttatcaacttgaaaaagtggcaccgagtcggtgctttt

SEQ ID NO: 316 - RNA, 100 nt, synthetic construct
  source 1..100: mol_type = other RNA
ctcagctagctgaggcccctgttttagagctagaaatagcaagttaaaataaggctagtccgttatcaacttgaaaaagtggcaccgagtcggtgctttt

SEQ ID NO: 317 - RNA, 100 nt, synthetic construct
  source 1..100: mol_type = other RNA
aagagacactccacctggatgttttagagctagaaatagcaagttaaaataaggctagtccgttatcaacttgaaaaagtggcaccgagtcggtgctttt

SEQ ID NO: 318 - RNA, 100 nt, synthetic construct
  source 1..100: mol_type = other RNA
gaaaagtcattgggaaccccgttttagagctagaaatagcaagttaaaataaggctagtccgttatcaacttgaaaaagtggcaccgagtcggtgctttt

SEQ ID NO: 319 - RNA, 100 nt, synthetic construct
  source 1..100: mol_type = other RNA
gccagtaggtgacttgggacgttttagagctagaaatagcaagttaaaataaggctagtccgttatcaacttgaaaaagtggcaccgagtcggtgctttt

SEQ ID NO: 320 - RNA, 100 nt, synthetic construct
  source 1..100: mol_type = other RNA
atcaaggcaccagactcaatgttttagagctagaaatagcaagttaaaataaggctagtccgttatcaacttgaaaaagtggcaccgagtcggtgctttt

SEQ ID NO: 321 - RNA, 100 nt, synthetic construct
  source 1..100: mol_type = other RNA
atcaggattgattagcaggcgttttagagctagaaatagcaagttaaaataaggctagtccgttatcaacttgaaaaagtggcaccgagtcggtgctttt

SEQ ID NO: 322 - RNA, 100 nt, synthetic construct
  source 1..100: mol_type = other RNA
tgccacaactaccaggtggggttttagagctagaaatagcaagttaaaataaggctagtccgttatcaacttgaaaaagtggcaccgagtcggtgctttt

SEQ ID NO: 323 - RNA, 100 nt, synthetic construct
  source 1..100: mol_type = other RNA
gacactccacctggatgggagttttagagctagaaatagcaagttaaaataaggctagtccgttatcaacttgaaaaagtggcaccgagtcggtgctttt

SEQ ID NO: 324 - RNA, 100 nt, synthetic construct
  source 1..100: mol_type = other RNA
gcagacccctgtcccatacagttttagagctagaaatagcaagttaaaataaggctagtccgttatcaacttgaaaaagtggcaccgagtcggtgctttt

SEQ ID NO: 325 - RNA, 100 nt, synthetic construct
  source 1..100: mol_type = other RNA
gggatcagctctgaccctgcgttttagagctagaaatagcaagttaaaataaggctagtccgttatcaacttgaaaaagtggcaccgagtcggtgctttt

SEQ ID NO: 326 - RNA, 100 nt, synthetic construct
  source 1..100: mol_type = other RNA
tcagctagctgaggcccctggttttagagctagaaatagcaagttaaaataaggctagtccgttatcaacttgaaaaagtggcaccgagtcggtgctttt

SEQ ID NO: 327 - RNA, 100 nt, synthetic construct
  source 1..100: mol_type = other RNA
ctggggctctgtatgccagagttttagagctagaaatagcaagttaaaataaggctagtccgttatcaacttgaaaaagtggcaccgagtcggtgctttt

SEQ ID NO: 328 - RNA, 100 nt, synthetic construct
  source 1..100: mol_type = other RNA
gagtaacagtgcagtgtgctgttttagagctagaaatagcaagttaaaataaggctagtccgttatcaacttgaaaaagtggcaccgagtcggtgctttt

SEQ ID NO: 329 - RNA, 100 nt, synthetic construct
  source 1..100: mol_type = other RNA
gtgcttctgcagggttgccagttttagagctagaaatagcaagttaaaataaggctagtccgttatcaacttgaaaaagtggcaccgagtcggtgctttt

SEQ ID NO: 330 - RNA, 100 nt, synthetic construct
  source 1..100: mol_type = other RNA
tcagagctgatcccctctgtgttttagagctagaaatagcaagttaaaataaggctagtccgttatcaacttgaaaaagtggcaccgagtcggtgctttt

SEQ ID NO: 331 - RNA, 100 nt, synthetic construct
  source 1..100: mol_type = other RNA
cgggaggcacagaaccaagggttttagagctagaaatagcaagttaaaataaggctagtccgttatcaacttgaaaaagtggcaccgagtcggtgctttt

SEQ ID NO: 332 - RNA, 100 nt, synthetic construct
  source 1..100: mol_type = other RNA
tgtctccctgaaaagtcattgttttagagctagaaatagcaagttaaaataaggctagtccgttatcaacttgaaaaagtggcaccgagtcggtgctttt

SEQ ID NO: 333 - RNA, 100 nt, synthetic construct
  source 1..100: mol_type = other RNA
aaagtcattgggaacccctggttttagagctagaaatagcaagttaaaataaggctagtccgttatcaacttgaaaaagtggcaccgagtcggtgctttt

SEQ ID NO: 334 - RNA, 100 nt, synthetic construct
  source 1..100: mol_type = other RNA
gtggtggatgctcctgccccgttttagagctagaaatagcaagttaaaataaggctagtccgttatcaacttgaaaaagtggcaccgagtcggtgctttt

SEQ ID NO: 335 - RNA, 100 nt, synthetic construct
  source 1..100: mol_type = other RNA
gtggaattctggcatctagtgttttagagctagaaatagcaagttaaaataaggctagtccgttatcaacttgaaaaagtggcaccgagtcggtgctttt

SEQ ID NO: 336 - RNA, 100 nt, synthetic construct
  source 1..100: mol_type = other RNA
cctgtggccagtaggtgactgttttagagctagaaatagcaagttaaaataaggctagtccgttatcaacttgaaaaagtggcaccgagtcggtgctttt

SEQ ID NO: 337 - RNA, 100 nt, synthetic construct
  source 1..100: mol_type = other RNA
atggatagaaaaacggtgaagttttagagctagaaatagcaagttaaaataaggctagtccgttatcaacttgaaaaagtggcaccgagtcggtgctttt

SEQ ID NO: 338 - RNA, 100 nt, synthetic construct
  source 1..100: mol_type = other RNA
ctccacctggatgggaaggtgttttagagctagaaatagcaagttaaaataaggctagtccgttatcaacttgaaaaagtggcaccgagtcggtgctttt

SEQ ID NO: 339 - RNA, 100 nt, synthetic construct
  source 1..100: mol_type = other RNA
actccacctggatgggaagggttttagagctagaaatagcaagttaaaataaggctagtccgttatcaacttgaaaaagtggcaccgagtcggtgctttt

SEQ ID NO: 340 - RNA, 91 nt, synthetic construct
  source 1..91: mol_type = other RNA
gagtgcgctttcagcagcgcgttttagagctagaaatagcaagttaaaataaggctagtccgttatcacgaaagggcaccgagtcggtgct

SEQ ID NO: 341 - RNA, 91 nt, synthetic construct
  source 1..91: mol_type = other RNA
cctctacactctaatttacggttttagagctagaaatagcaagttaaaataaggctagtccgttatcacgaaagggcaccgagtcggtgct

SEQ ID NO: 342 - RNA, 91 nt, synthetic construct
  source 1..91: mol_type = other RNA
cagcgggggtggccccgtatgttttagagctagaaatagcaagttaaaataaggctagtccgttatcacgaaagggcaccgagtcggtgct

SEQ ID NO: 343 - RNA, 91 nt, synthetic construct
  source 1..91: mol_type = other RNA
agctagctgagacctatacggttttagagctagaaatagcaagttaaaataaggctagtccgttatcacgaaagggcaccgagtcggtgct

SEQ ID NO: 344 - RNA, 91 nt, synthetic construct
  source 1..91: mol_type = other RNA
ccacgtaaattagagtgtaggttttagagctagaaatagcaagttaaaataaggctagtccgttatcacgaaagggcaccgagtcggtgct

SEQ ID NO: 345 - RNA, 91 nt, synthetic construct
  source 1..91: mol_type = other RNA
cactcgaatcttcaacactcgttttagagctagaaatagcaagttaaaataaggctagtccgttatcacgaaagggcaccgagtcggtgct

SEQ ID NO: 346 - RNA, 91 nt, synthetic construct
  source 1..91: mol_type = other RNA
actcgaatcttcaacactcagttttagagctagaaatagcaagttaaaataaggctagtccgttatcacgaaagggcaccgagtcggtgct

SEQ ID NO: 347 - RNA, 91 nt, synthetic construct
  source 1..91: mol_type = other RNA
gtttcacagaacggtgcatcgttttagagctagaaatagcaagttaaaataaggctagtccgttatcacgaaagggcaccgagtcggtgct

SEQ ID NO: 348 - RNA, 91 nt, synthetic construct
  source 1..91: mol_type = other RNA
ggttgccacaactaccaggtgttttagagctagaaatagcaagttaaaataaggctagtccgttatcacgaaagggcaccgagtcggtgct

SEQ ID NO: 349 - RNA, 91 nt, synthetic construct
  source 1..91: mol_type = other RNA
tggccttgttcccgtgtggcgttttagagctagaaatagcaagttaaaataaggctagtccgttatcacgaaagggcaccgagtcggtgct

SEQ ID NO: 350 - RNA, 91 nt, synthetic construct
  source 1..91: mol_type = other RNA
cggccagccacacgggaacagttttagagctagaaatagcaagttaaaataaggctagtccgttatcacgaaagggcaccgagtcggtgct

SEQ ID NO: 351 - RNA, 91 nt, synthetic construct
  source 1..91: mol_type = other RNA
aaacaaggcatcacatagcggttttagagctagaaatagcaagttaaaataaggctagtccgttatcacgaaagggcaccgagtcggtgct

SEQ ID NO: 352 - RNA, 91 nt, synthetic construct
  source 1..91: mol_type = other RNA
gtataggtctcagctagctggttttagagctagaaatagcaagttaaaataaggctagtccgttatcacgaaagggcaccgagtcggtgct

SEQ ID NO: 353 - RNA, 91 nt, synthetic construct
  source 1..91: mol_type = other RNA
ggtgccttgattgtgaatctgttttagagctagaaatagcaagttaaaataaggctagtccgttatcacgaaagggcaccgagtcggtgct

SEQ ID NO: 354 - RNA, 91 nt, synthetic construct
  source 1..91: mol_type = other RNA
gtgcatcaggattgattagcgttttagagctagaaatagcaagttaaaataaggctagtccgttatcacgaaagggcaccgagtcggtgct

SEQ ID NO: 355 - RNA, 91 nt, synthetic construct
  source 1..91: mol_type = other RNA
ggggttcccaatgacttttcgttttagagctagaaatagcaagttaaaataaggctagtccgttatcacgaaagggcaccgagtcggtgct

SEQ ID NO: 356 - RNA, 91 nt, synthetic construct
  source 1..91: mol_type = other RNA
tcagctagctgagacctatagttttagagctagaaatagcaagttaaaataaggctagtccgttatcacgaaagggcaccgagtcggtgct

SEQ ID NO: 357 - RNA, 91 nt, synthetic construct
  source 1..91: mol_type = other RNA
gatgcaccgttctgtgaaacgttttagagctagaaatagcaagttaaaataaggctagtccgttatcacgaaagggcaccgagtcggtgct

SEQ ID NO: 358 - RNA, 91 nt, synthetic construct
  source 1..91: mol_type = other RNA
gacggttttgtccctgttgggttttagagctagaaatagcaagttaaaataaggctagtccgttatcacgaaagggcaccgagtcggtgct

SEQ ID NO: 359 - RNA, 91 nt, synthetic construct
  source 1..91: mol_type = other RNA
cttgaaatgtagacccccaggttttagagctagaaatagcaagttaaaataaggctagtccgttatcacgaaagggcaccgagtcggtgct

SEQ ID NO: 360 - RNA, 91 nt, synthetic construct
  source 1..91: mol_type = other RNA
aagaagcaccaggtacagcggttttagagctagaaatagcaagttaaaataaggctagtccgttatcacgaaagggcaccgagtcggtgct

SEQ ID NO: 361 - RNA, 91 nt, synthetic construct
  source 1..91: mol_type = other RNA
ttgccacaactaccaggtgggttttagagctagaaatagcaagttaaaataaggctagtccgttatcacgaaagggcaccgagtcggtgct

SEQ ID NO: 362 - RNA, 91 nt, synthetic construct
  source 1..91: mol_type = other RNA
ttcttgaaatgtagacccccgttttagagctagaaatagcaagttaaaataaggctagtccgttatcacgaaagggcaccgagtcggtgct

SEQ ID NO: 363 - RNA, 91 nt, synthetic construct
  source 1..91: mol_type = other RNA
agcaccaggtacagcggggggttttagagctagaaatagcaagttaaaataaggctagtccgttatcacgaaagggcaccgagtcggtgct

SEQ ID NO: 364 - RNA, 91 nt, synthetic construct
  source 1..91: mol_type = other RNA
ttatgatgaacagtgtcatcgttttagagctagaaatagcaagttaaaataaggctagtccgttatcacgaaagggcaccgagtcggtgct

SEQ ID NO: 365 - RNA, 91 nt, synthetic construct
  source 1..91: mol_type = other RNA
catgttaaaatgaacacggggttttagagctagaaatagcaagttaaaataaggctagtccgttatcacgaaagggcaccgagtcggtgct

SEQ ID NO: 366 - RNA, 91 nt, synthetic construct
  source 1..91: mol_type = other RNA
gaagagacactccacctggagttttagagctagaaatagcaagttaaaataaggctagtccgttatcacgaaagggcaccgagtcggtgct

SEQ ID NO: 367 - RNA, 91 nt, synthetic construct
  source 1..91: mol_type = other RNA
tctgctctccagcactcggggttttagagctagaaatagcaagttaaaataaggctagtccgttatcacgaaagggcaccgagtcggtgct

SEQ ID NO: 368 - RNA, 91 nt, synthetic construct
  source 1..91: mol_type = other RNA
gtctgagcagtagtccaggagttttagagctagaaatagcaagttaaaataaggctagtccgttatcacgaaagggcaccgagtcggtgct

SEQ ID NO: 369 - RNA, 91 nt, synthetic construct
  source 1..91: mol_type = other RNA
atctggcatacagagccccagttttagagctagaaatagcaagttaaaataaggctagtccgttatcacgaaagggcaccgagtcggtgct

SEQ ID NO: 370 - RNA, 91 nt, synthetic construct
  source 1..91: mol_type = other RNA
cacaactaccaggtgggggggttttagagctagaaatagcaagttaaaataaggctagtccgttatcacgaaagggcaccgagtcggtgct

SEQ ID NO: 371 - RNA, 91 nt, synthetic construct
  source 1..91: mol_type = other RNA
agagtaacagtgcagtgtgcgttttagagctagaaatagcaagttaaaataaggctagtccgttatcacgaaagggcaccgagtcggtgct

SEQ ID NO: 372 - RNA, 91 nt, synthetic construct
  source 1..91: mol_type = other RNA
tttaagagtcgagaaattctgttttagagctagaaatagcaagttaaaataaggctagtccgttatcacgaaagggcaccgagtcggtgct

SEQ ID NO: 373 - RNA, 91 nt, synthetic construct
  source 1..91: mol_type = other RNA
gttttgtccctgttgggggtgttttagagctagaaatagcaagttaaaataaggctagtccgttatcacgaaagggcaccgagtcggtgct

SEQ ID NO: 374 - RNA, 91 nt, synthetic construct
  source 1..91: mol_type = other RNA
gtggctggccgtcctgtgcagttttagagctagaaatagcaagttaaaataaggctagtccgttatcacgaaagggcaccgagtcggtgct

SEQ ID NO: 375 - RNA, 91 nt, synthetic construct
  source 1..91: mol_type = other RNA
catctggcatacagagccccgttttagagctagaaatagcaagttaaaataaggctagtccgttatcacgaaagggcaccgagtcggtgct

SEQ ID NO: 376 - RNA, 91 nt, synthetic construct
  source 1..91: mol_type = other RNA
ctgtggccagtaggtgacttgttttagagctagaaatagcaagttaaaataaggctagtccgttatcacgaaagggcaccgagtcggtgct

SEQ ID NO: 377 - RNA, 91 nt, synthetic construct
  source 1..91: mol_type = other RNA
tcttgcatataacattaggagttttagagctagaaatagcaagttaaaataaggctagtccgttatcacgaaagggcaccgagtcggtgct

SEQ ID NO: 378 - RNA, 91 nt, synthetic construct
  source 1..91: mol_type = other RNA
atgaaggggcacaagtggcagttttagagctagaaatagcaagttaaaataaggctagtccgttatcacgaaagggcaccgagtcggtgct

SEQ ID NO: 379 - RNA, 91 nt, synthetic construct
  source 1..91: mol_type = other RNA
aaaagtcattgggaacccctgttttagagctagaaatagcaagttaaaataaggctagtccgttatcacgaaagggcaccgagtcggtgct

SEQ ID NO: 380 - RNA, 91 nt, synthetic construct
  source 1..91: mol_type = other RNA
gtggagggattaatgacagagttttagagctagaaatagcaagttaaaataaggctagtccgttatcacgaaagggcaccgagtcggtgct

SEQ ID NO: 381 - RNA, 91 nt, synthetic construct
  source 1..91: mol_type = other RNA
ctgcagaagcaccccacagagttttagagctagaaatagcaagttaaaataaggctagtccgttatcacgaaagggcaccgagtcggtgct

SEQ ID NO: 382 - RNA, 91 nt, synthetic construct
  source 1..91: mol_type = other RNA
aggcaaggacaggggccatcgttttagagctagaaatagcaagttaaaataaggctagtccgttatcacgaaagggcaccgagtcggtgct

SEQ ID NO: 383 - RNA, 91 nt, synthetic construct
  source 1..91: mol_type = other RNA
tctggcatacagagccccaggttttagagctagaaatagcaagttaaaataaggctagtccgttatcacgaaagggcaccgagtcggtgct

SEQ ID NO: 384 - RNA, 91 nt, synthetic construct
  source 1..91: mol_type = other RNA
gaagaagtaggcaaggacaggttttagagctagaaatagcaagttaaaataaggctagtccgttatcacgaaagggcaccgagtcggtgct

SEQ ID NO: 385 - RNA, 91 nt, synthetic construct
  source 1..91: mol_type = other RNA
agaaccattcctctggatcggttttagagctagaaatagcaagttaaaataaggctagtccgttatcacgaaagggcaccgagtcggtgct

SEQ ID NO: 386 - RNA, 91 nt, synthetic construct
  source 1..91: mol_type = other RNA
cagctagctgagacctatacgttttagagctagaaatagcaagttaaaataaggctagtccgttatcacgaaagggcaccgagtcggtgct

SEQ ID NO: 387 - RNA, 91 nt, synthetic construct
  source 1..91: mol_type = other RNA
ggttgtctgagcagtagtccgttttagagctagaaatagcaagttaaaataaggctagtccgttatcacgaaagggcaccgagtcggtgct

SEQ ID NO: 388 - RNA, 91 nt, synthetic construct
  source 1..91: mol_type = other RNA
gaaaccccgatccagaggaagttttagagctagaaatagcaagttaaaataaggctagtccgttatcacgaaagggcaccgagtcggtgct

SEQ ID NO: 389 - RNA, 91 nt, synthetic construct
  source 1..91: mol_type = other RNA
tcacatagcgaggaagccctgttttagagctagaaatagcaagttaaaataaggctagtccgttatcacgaaagggcaccgagtcggtgct

SEQ ID NO: 390 - RNA, 91 nt, synthetic construct
  source 1..91: mol_type = other RNA
cagagaactccaattgagtcgttttagagctagaaatagcaagttaaaataaggctagtccgttatcacgaaagggcaccgagtcggtgct

SEQ ID NO: 391 - RNA, 91 nt, synthetic construct
  source 1..91: mol_type = other RNA
gggacaaaaccgtctctggtgttttagagctagaaatagcaagttaaaataaggctagtccgttatcacgaaagggcaccgagtcggtgct

SEQ ID NO: 392 - RNA, 91 nt, synthetic construct
  source 1..91: mol_type = other RNA
atcacatagcgaggaagcccgttttagagctagaaatagcaagttaaaataaggctagtccgttatcacgaaagggcaccgagtcggtgct

SEQ ID NO: 393 - RNA, 91 nt, synthetic construct
  source 1..91: mol_type = other RNA
ataaatgccctgtatgggacgttttagagctagaaatagcaagttaaaataaggctagtccgttatcacgaaagggcaccgagtcggtgct

SEQ ID NO: 394 - RNA, 91 nt, synthetic construct
  source 1..91: mol_type = other RNA
agacggttttgtccctgttggttttagagctagaaatagcaagttaaaataaggctagtccgttatcacgaaagggcaccgagtcggtgct

SEQ ID NO: 395 - RNA, 91 nt, synthetic construct
  source 1..91: mol_type = other RNA
aaatgccctgtatgggacaggttttagagctagaaatagcaagttaaaataaggctagtccgttatcacgaaagggcaccgagtcggtgct

SEQ ID NO: 396 - RNA, 91 nt, synthetic construct
  source 1..91: mol_type = other RNA
tgattgtgaatctaggcatggttttagagctagaaatagcaagttaaaataaggctagtccgttatcacgaaagggcaccgagtcggtgct

SEQ ID NO: 397 - RNA, 91 nt, synthetic construct
  source 1..91: mol_type = other RNA
gagacggttttgtccctgttgttttagagctagaaatagcaagttaaaataaggctagtccgttatcacgaaagggcaccgagtcggtgct

SEQ ID NO: 398 - RNA, 91 nt, synthetic construct
  source 1..91: mol_type = other RNA
actgagaaaccccgatccaggttttagagctagaaatagcaagttaaaataaggctagtccgttatcacgaaagggcaccgagtcggtgct

SEQ ID NO: 399 - RNA, 91 nt, synthetic construct
  source 1..91: mol_type = other RNA
catagcgaggaagccctggggttttagagctagaaatagcaagttaaaataaggctagtccgttatcacgaaagggcaccgagtcggtgct

SEQ ID NO: 400 - RNA, 91 nt, synthetic construct
  source 1..91: mol_type = other RNA
acattctagttccaggcctggttttagagctagaaatagcaagttaaaataaggctagtccgttatcacgaaagggcaccgagtcggtgct

SEQ ID NO: 401 - RNA, 91 nt, synthetic construct
  source 1..91: mol_type = other RNA
actcgggaggcacagaaccagttttagagctagaaatagcaagttaaaataaggctagtccgttatcacgaaagggcaccgagtcggtgct

SEQ ID NO: 402 - RNA, 91 nt, synthetic construct
  source 1..91: mol_type = other RNA
catgcctagattcacaatcagttttagagctagaaatagcaagttaaaataaggctagtccgttatcacgaaagggcaccgagtcggtgct

SEQ ID NO: 403 - RNA, 91 nt, synthetic construct
  source 1..91: mol_type = other RNA
ggaactagaatgtccctcctgttttagagctagaaatagcaagttaaaataaggctagtccgttatcacgaaagggcaccgagtcggtgct

SEQ ID NO: 404 - RNA, 91 nt, synthetic construct
  source 1..91: mol_type = other RNA
ctcagctagctgaggcccctgttttagagctagaaatagcaagttaaaataaggctagtccgttatcacgaaagggcaccgagtcggtgct

SEQ ID NO: 405 - RNA, 91 nt, synthetic construct
  source 1..91: mol_type = other RNA
aagagacactccacctggatgttttagagctagaaatagcaagttaaaataaggctagtccgttatcacgaaagggcaccgagtcggtgct

SEQ ID NO: 406 - RNA, 91 nt, synthetic construct
  source 1..91: mol_type = other RNA
gaaaagtcattgggaaccccgttttagagctagaaatagcaagttaaaataaggctagtccgttatcacgaaagggcaccgagtcggtgct

SEQ ID NO: 407 - RNA, 91 nt, synthetic construct
  source 1..91: mol_type = other RNA
gccagtaggtgacttgggacgttttagagctagaaatagcaagttaaaataaggctagtccgttatcacgaaagggcaccgagtcggtgct

SEQ ID NO: 408 - RNA, 91 nt, synthetic construct
  source 1..91: mol_type = other RNA
atcaaggcaccagactcaatgttttagagctagaaatagcaagttaaaataaggctagtccgttatcacgaaagggcaccgagtcggtgct

SEQ ID NO: 409 - RNA, 91 nt, synthetic construct
  source 1..91: mol_type = other RNA
atcaggattgattagcaggcgttttagagctagaaatagcaagttaaaataaggctagtccgttatcacgaaagggcaccgagtcggtgct

SEQ ID NO: 410 - RNA, 91 nt, synthetic construct
  source 1..91: mol_type = other RNA
tgccacaactaccaggtggggttttagagctagaaatagcaagttaaaataaggctagtccgttatcacgaaagggcaccgagtcggtgct

SEQ ID NO: 411 - RNA, 91 nt, synthetic construct
  source 1..91: mol_type = other RNA
gacactccacctggatgggagttttagagctagaaatagcaagttaaaataaggctagtccgttatcacgaaagggcaccgagtcggtgct

SEQ ID NO: 412 - RNA, 91 nt, synthetic construct
  source 1..91: mol_type = other RNA
gcagacccctgtcccatacagttttagagctagaaatagcaagttaaaataaggctagtccgttatcacgaaagggcaccgagtcggtgct

SEQ ID NO: 413 - RNA, 91 nt, synthetic construct
  source 1..91: mol_type = other RNA
gggatcagctctgaccctgcgttttagagctagaaatagcaagttaaaataaggctagtccgttatcacgaaagggcaccgagtcggtgct

SEQ ID NO: 414 - RNA, 91 nt, synthetic construct
  source 1..91: mol_type = other RNA
tcagctagctgaggcccctggttttagagctagaaatagcaagttaaaataaggctagtccgttatcacgaaagggcaccgagtcggtgct

SEQ ID NO: 415 - RNA, 91 nt, synthetic construct
  source 1..91: mol_type = other RNA
ctggggctctgtatgccagagttttagagctagaaatagcaagttaaaataaggctagtccgttatcacgaaagggcaccgagtcggtgct

SEQ ID NO: 416 - RNA, 91 nt, synthetic construct
  source 1..91: mol_type = other RNA
gagtaacagtgcagtgtgctgttttagagctagaaatagcaagttaaaataaggctagtccgttatcacgaaagggcaccgagtcggtgct

SEQ ID NO: 417 - RNA, 91 nt, synthetic construct
  source 1..91: mol_type = other RNA
gtgcttctgcagggttgccagttttagagctagaaatagcaagttaaaataaggctagtccgttatcacgaaagggcaccgagtcggtgct

SEQ ID NO: 418 - RNA, 91 nt, synthetic construct
  source 1..91: mol_type = other RNA
tcagagctgatcccctctgtgttttagagctagaaatagcaagttaaaataaggctagtccgttatcacgaaagggcaccgagtcggtgct

SEQ ID NO: 419 - RNA, 91 nt, synthetic construct
  source 1..91: mol_type = other RNA
cgggaggcacagaaccaagggttttagagctagaaatagcaagttaaaataaggctagtccgttatcacgaaagggcaccgagtcggtgct

SEQ ID NO: 420 - RNA, 91 nt, synthetic construct
  source 1..91: mol_type = other RNA
tgtctccctgaaaagtcattgttttagagctagaaatagcaagttaaaataaggctagtccgttatcacgaaagggcaccgagtcggtgct

SEQ ID NO: 421 - RNA, 91 nt, synthetic construct
  source 1..91: mol_type = other RNA
aaagtcattgggaacccctggttttagagctagaaatagcaagttaaaataaggctagtccgttatcacgaaagggcaccgagtcggtgct

SEQ ID NO: 422 - RNA, 91 nt, synthetic construct
  source 1..91: mol_type = other RNA
gtggtggatgctcctgccccgttttagagctagaaatagcaagttaaaataaggctagtccgttatcacgaaagggcaccgagtcggtgct

SEQ ID NO: 423 - RNA, 91 nt, synthetic construct
  source 1..91: mol_type = other RNA
gtggaattctggcatctagtgttttagagctagaaatagcaagttaaaataaggctagtccgttatcacgaaagggcaccgagtcggtgct

SEQ ID NO: 424 - RNA, 91 nt, synthetic construct
  source 1..91: mol_type = other RNA
cctgtggccagtaggtgactgttttagagctagaaatagcaagttaaaataaggctagtccgttatcacgaaagggcaccgagtcggtgct

SEQ ID NO: 425 - RNA, 91 nt, synthetic construct
  source 1..91: mol_type = other RNA
atggatagaaaaacggtgaagttttagagctagaaatagcaagttaaaataaggctagtccgttatcacgaaagggcaccgagtcggtgct

SEQ ID NO: 426 - RNA, 91 nt, synthetic construct
  source 1..91: mol_type = other RNA
ctccacctggatgggaaggtgttttagagctagaaatagcaagttaaaataaggctagtccgttatcacgaaagggcaccgagtcggtgct

SEQ ID NO: 427 - RNA, 91 nt, synthetic construct
  source 1..91: mol_type = other RNA
actccacctggatgggaagggttttagagctagaaatagcaagttaaaataaggctagtccgttatcacgaaagggcaccgagtcggtgct

SEQ ID NO: 428 - RNA, 71 nt, synthetic construct
  source 1..71: mol_type = other RNA
gttttagagctagaaatagcaagttaaaataaggctagtccgttatcacgaaagggcaccgagtcggtgct

SEQ ID NO: 429 - RNA, 91 nt, synthetic construct
  source 1..91: mol_type = other RNA
nnnnnnnnnnnnnnnnnnnngttttagagctagaaatagcaagttaaaataaggctagtccgttatcacgaaagggcaccgagtcggtgct

SEQ ID NO: 430 - RNA, 20 nt, synthetic construct
  source 1..20: mol_type = other RNA
catagggactaatgcgtggt

SEQ ID NO: 431 - RNA, 20 nt, synthetic construct
  source 1..20: mol_type = other RNA
caccgtcactcattcaagcc

SEQ ID NO: 432 - RNA, 20 nt, synthetic construct
  source 1..20: mol_type = other RNA
gtggccaggttcagtcgaag

SEQ ID NO: 433 - RNA, 20 nt, synthetic construct
  source 1..20: mol_type = other RNA
aatgcgtggtgggtccccat

SEQ ID NO: 434 - RNA, 20 nt, synthetic construct
  source 1..20: mol_type = other RNA
agctcctcttcgactgaacc

SEQ ID NO: 435 - RNA, 20 nt, synthetic construct
  source 1..20: mol_type = other RNA
ccatagggactaatgcgtgg

SEQ ID NO: 436 - RNA, 20 nt, synthetic construct
  source 1..20: mol_type = other RNA
cagccatagggactaatgcg

SEQ ID NO: 437 - RNA, 20 nt, synthetic construct
  source 1..20: mol_type = other RNA
tcattcggctcaccagaccc

SEQ ID NO: 438 - RNA, 20 nt, synthetic construct
  source 1..20: mol_type = other RNA
ggtctggtgagccgaatgaa

SEQ ID NO: 439 - RNA, 20 nt, synthetic construct
  source 1..20: mol_type = other RNA
tggcttgtttgccggcatat

SEQ ID NO: 440 - RNA, 20 nt, synthetic construct
  source 1..20: mol_type = other RNA
aggagggcttacccccagta

SEQ ID NO: 441 - RNA, 20 nt, synthetic construct
  source 1..20: mol_type = other RNA
gccggcaaacaagccacttt

SEQ ID NO: 442 - RNA, 20 nt, synthetic construct
  source 1..20: mol_type = other RNA
gagctgggcgtatagacccc

SEQ ID NO: 443 - RNA, 20 nt, synthetic construct
  source 1..20: mol_type = other RNA
gcatcatggggtgcgaggct

SEQ ID NO: 444 - RNA, 20 nt, synthetic construct
  source 1..20: mol_type = other RNA
agctgggcgtatagacccct

SEQ ID NO: 445 - RNA, 20 nt, synthetic construct
  source 1..20: mol_type = other RNA
taatgcgtggtgggtcccca

SEQ ID NO: 446 - RNA, 20 nt, synthetic construct
  source 1..20: mol_type = other RNA
aaggagggcttacccccagt

SEQ ID NO: 447 - RNA, 20 nt, synthetic construct
  source 1..20: mol_type = other RNA
catcatggggtgcgaggctt

SEQ ID NO: 448 - RNA, 20 nt, synthetic construct
  source 1..20: mol_type = other RNA
tctcgaagcctgtcttgaac

SEQ ID NO: 449 - RNA, 20 nt, synthetic construct
  source 1..20: mol_type = other RNA
ttgggagctggattgtagcg

SEQ ID NO: 450 - RNA, 20 nt, synthetic construct
  source 1..20: mol_type = other RNA
gggtctggtgagccgaatga

SEQ ID NO: 451 - RNA, 20 nt, synthetic construct
  source 1..20: mol_type = other RNA
agtccctatggctgcatcat

SEQ ID NO: 452 - RNA, 20 nt, synthetic construct
  source 1..20: mol_type = other RNA
tgggggtaagccctcctttt

SEQ ID NO: 453 - RNA, 20 nt, synthetic construct
  source 1..20: mol_type = other RNA
ttacccccagtagggcccat

SEQ ID NO: 454 - RNA, 20 nt, synthetic construct
  source 1..20: mol_type = other RNA
ttgagagggatgactcctat

SEQ ID NO: 455 - RNA, 20 nt, synthetic construct
  source 1..20: mol_type = other RNA
gggggtaagccctccttttt

SEQ ID NO: 456 - RNA, 20 nt, synthetic construct
  source 1..20: mol_type = other RNA
tgacaagctaatctttgcac

SEQ ID NO: 457 - RNA, 20 nt, synthetic construct
  source 1..20: mol_type = other RNA
gcccaatgcccttgtcattc

SEQ ID NO: 458 - RNA, 20 nt, synthetic construct
  source 1..20: mol_type = other RNA
tacccccagtagggcccatg

SEQ ID NO: 459 - RNA, 20 nt, synthetic construct
  source 1..20: mol_type = other RNA
cctagtcaaaggccatgcct

SEQ ID NO: 460 - RNA, 20 nt, synthetic construct
  source 1..20: mol_type = other RNA
cacagcggggcaatcagagc

SEQ ID NO: 461 - RNA, 20 nt, synthetic construct
  source 1..20: mol_type = other RNA
tagacccctgggctaccacc

SEQ ID NO: 462 - RNA, 20 nt, synthetic construct
  source 1..20: mol_type = other RNA
atcccgcccactgaaggagt

SEQ ID NO: 463 - RNA, 20 nt, synthetic construct
  source 1..20: mol_type = other RNA
tgcaggctgacagcatactc

SEQ ID NO: 464 - RNA, 20 nt, synthetic construct
  source 1..20: mol_type = other RNA
cagaaccaggtgatccccca

SEQ ID NO: 465 - RNA, 20 nt, synthetic construct
  source 1..20: mol_type = other RNA
gggtgtatccatctcactgt

SEQ ID NO: 466 - RNA, 20 nt, synthetic construct
  source 1..20: mol_type = other RNA
gggtccccatgggccctact

SEQ ID NO: 467 - RNA, 20 nt, synthetic construct
  source 1..20: mol_type = other RNA
caaggcatggcctttgacta

SEQ ID NO: 468 - RNA, 20 nt, synthetic construct
  source 1..20: mol_type = other RNA
gatctatctccaactctact

SEQ ID NO: 469 - RNA, 20 nt, synthetic construct
  source 1..20: mol_type = other RNA
cagtgagatggatacaccct

SEQ ID NO: 470 - RNA, 20 nt, synthetic construct
  source 1..20: mol_type = other RNA
gaagaaatcccgcccactga

SEQ ID NO: 471 - RNA, 20 nt, synthetic construct
  source 1..20: mol_type = other RNA
atcccccaggtggtagccca

SEQ ID NO: 472 - RNA, 20 nt, synthetic construct
  source 1..20: mol_type = other RNA
gtccccatgggccctactgg

SEQ ID NO: 473 - RNA, 20 nt, synthetic construct
  source 1..20: mol_type = other RNA
aatcccccaggtggtagccc

SEQ ID NO: 474 - RNA, 20 nt, synthetic construct
  source 1..20: mol_type = other RNA
catgagcctactccttcagt

SEQ ID NO: 475 - RNA, 20 nt, synthetic construct
  source 1..20: mol_type = other RNA
tcctagttccagttcaagac

SEQ ID NO: 476 - RNA, 20 nt, synthetic construct
  source 1..20: mol_type = other RNA
ggtccccatgggccctactg

SEQ ID NO: 477 - RNA, 20 nt, synthetic construct
  source 1..20: mol_type = other RNA
ccaccacgcattagtcccta

SEQ ID NO: 478 - RNA, 20 nt, synthetic construct
  source 1..20: mol_type = other RNA
tgggtggaccccattccaca

SEQ ID NO: 479 - RNA, 20 nt, synthetic construct
  source 1..20: mol_type = other RNA
agttagacatgccaatatgc

SEQ ID NO: 480 - RNA, 20 nt, synthetic construct
  source 1..20: mol_type = other RNA
agtgcaaagattagcttgtc

SEQ ID NO: 481 - RNA, 20 nt, synthetic construct
  source 1..20: mol_type = other RNA
tgggtccccatgggccctac

SEQ ID NO: 482 - RNA, 20 nt, synthetic construct
  source 1..20: mol_type = other RNA
atgcccttgtcattcaggtt

SEQ ID NO: 483 - RNA, 20 nt, synthetic construct
  source 1..20: mol_type = other RNA
tagtccctatggctgcatca

SEQ ID NO: 484 - RNA, 20 nt, synthetic construct
  source 1..20: mol_type = other RNA
gcctgtcttgaactggaact

SEQ ID NO: 485 - RNA, 20 nt, synthetic construct
  source 1..20: mol_type = other RNA
caaccatctgctgtcattgc

SEQ ID NO: 486 - RNA, 20 nt, synthetic construct
  source 1..20: mol_type = other RNA
acagtgagatggatacaccc

SEQ ID NO: 487 - RNA, 20 nt, synthetic construct
  source 1..20: mol_type = other RNA
tttgagagggatgactccta

SEQ ID NO: 488 - RNA, 20 nt, synthetic construct
  source 1..20: mol_type = other RNA
gccaaaagtggcttgtttgc

SEQ ID NO: 489 - RNA, 20 nt, synthetic construct
  source 1..20: mol_type = other RNA
gattgtagcgtggtgatggc

SEQ ID NO: 490 - RNA, 20 nt, synthetic construct
  source 1..20: mol_type = other RNA
ccactgaaggagtaggctca

SEQ ID NO: 491 - RNA, 20 nt, synthetic construct
  source 1..20: mol_type = other RNA
ggacaggttgcaggacacgc

SEQ ID NO: 492 - RNA, 20 nt, synthetic construct
  source 1..20: mol_type = other RNA
gcctgaagccatcctacccc

SEQ ID NO: 493 - RNA, 20 nt, synthetic construct
  source 1..20: mol_type = other RNA
tcactgtaggtagcacagcg

SEQ ID NO: 494 - RNA, 20 nt, synthetic construct
  source 1..20: mol_type = other RNA
ccaaggcatggcctttgact

SEQ ID NO: 495 - RNA, 20 nt, synthetic construct
  source 1..20: mol_type = other RNA
acctgaatgacaagggcatt

SEQ ID NO: 496 - RNA, 20 nt, synthetic construct
  source 1..20: mol_type = other RNA
ccatgagcctactccttcag

SEQ ID NO: 497 - RNA, 20 nt, synthetic construct
  source 1..20: mol_type = other RNA
aaacatggcagtgtctagac

SEQ ID NO: 498 - RNA, 20 nt, synthetic construct
  source 1..20: mol_type = other RNA
gggatgactcctatgggccc

SEQ ID NO: 499 - RNA, 20 nt, synthetic construct
  source 1..20: mol_type = other RNA
gcttccatgggttggagcat

SEQ ID NO: 500 - RNA, 20 nt, synthetic construct
  source 1..20: mol_type = other RNA
cttacccccagtagggccca

SEQ ID NO: 501 - RNA, 20 nt, synthetic construct
  source 1..20: mol_type = other RNA
gtgaactcagggctcccccc

SEQ ID NO: 502 - RNA, 20 nt, synthetic construct
  source 1..20: mol_type = other RNA
gtccctatggctgcatcatg

SEQ ID NO: 503 - RNA, 20 nt, synthetic construct
  source 1..20: mol_type = other RNA
ggcatggcctttgactaggg

SEQ ID NO: 504 - RNA, 20 nt, synthetic construct
  source 1..20: mol_type = other RNA
gcaccccatgatgcagccat

SEQ ID NO: 505 - RNA, 20 nt, synthetic construct
  source 1..20: mol_type = other RNA
atgagtgacggtgagcagac

SEQ ID NO: 506 - RNA, 20 nt, synthetic construct
  source 1..20: mol_type = other RNA
tggaagaatctcacctctcc

SEQ ID NO: 507 - RNA, 20 nt, synthetic construct
  source 1..20: mol_type = other RNA
ctgggtggaccccattccac

SEQ ID NO: 508 - RNA, 20 nt, synthetic construct
  source 1..20: mol_type = other RNA
tgacagcatactctggagat

SEQ ID NO: 509 - RNA, 20 nt, synthetic construct
  source 1..20: mol_type = other RNA
cagctctcctccctagtcaa

SEQ ID NO: 510 - RNA, 20 nt, synthetic construct
  source 1..20: mol_type = other RNA
ccagaaccaggtgatccccc

SEQ ID NO: 511 - RNA, 20 nt, synthetic construct
  source 1..20: mol_type = other RNA
agccaggtgatggaggcgcg

SEQ ID NO: 512 - RNA, 20 nt, synthetic construct
  source 1..20: mol_type = other RNA
cctttcctttttgcgtaagt

SEQ ID NO: 513 - RNA, 20 nt, synthetic construct
  source 1..20: mol_type = other RNA
tctggaaggcatctcaagga

SEQ ID NO: 514 - RNA, 20 nt, synthetic construct
  source 1..20: mol_type = other RNA
aacatggcagtgtctagaca

SEQ ID NO: 515 - RNA, 20 nt, synthetic construct
  source 1..20: mol_type = other RNA
tgaactcagggctcccccca

SEQ ID NO: 516 - RNA, 20 nt, synthetic construct
  source 1..20: mol_type = other RNA
agacccctgggctaccacct

SEQ ID NO: 517 - RNA, 20 nt, synthetic construct
  source 1..20: mol_type = other RNA
tcagccaatgctccaaccca

SEQ ID NO: 518 - DNA, 20 bp, synthetic construct
  source 1..20: mol_type = other DNA
catagggactaatgcgtggt

SEQ ID NO: 519 - DNA, 20 bp, synthetic construct
  source 1..20: mol_type = other DNA
caccgtcactcattcaagcc

SEQ ID NO: 520 - DNA, 20 bp, synthetic construct
  source 1..20: mol_type = other DNA
gtggccaggttcagtcgaag

SEQ ID NO: 521 - DNA, 20 bp, synthetic construct
  source 1..20: mol_type = other DNA
aatgcgtggtgggtccccat

SEQ ID NO: 522 - DNA, 20 bp, synthetic construct
  source 1..20: mol_type = other DNA
agctcctcttcgactgaacc

SEQ ID NO: 523 - DNA, 20 bp, synthetic construct
  source 1..20: mol_type = other DNA
ccatagggactaatgcgtgg

SEQ ID NO: 524 - DNA, 20 bp, synthetic construct
  source 1..20: mol_type = other DNA
cagccatagggactaatgcg

SEQ ID NO: 525 - DNA, 20 bp, synthetic construct
  source 1..20: mol_type = other DNA
tcattcggctcaccagaccc

SEQ ID NO: 526 - DNA, 20 bp, synthetic construct
  source 1..20: mol_type = other DNA
ggtctggtgagccgaatgaa

SEQ ID NO: 527 - DNA, 20 bp, synthetic construct
  source 1..20: mol_type = other DNA
tggcttgtttgccggcatat

SEQ ID NO: 528 - DNA, 20 bp, synthetic construct
  source 1..20: mol_type = other DNA
aggagggcttacccccagta

SEQ ID NO: 529 - DNA, 20 bp, synthetic construct
  source 1..20: mol_type = other DNA
gccggcaaacaagccacttt

SEQ ID NO: 530 - DNA, 20 bp, synthetic construct
  source 1..20: mol_type = other DNA
gagctgggcgtatagacccc

SEQ ID NO: 531 - DNA, 20 bp, synthetic construct
  source 1..20: mol_type = other DNA
gcatcatggggtgcgaggct

SEQ ID NO: 532 - DNA, 20 bp, synthetic construct
  source 1..20: mol_type = other DNA
agctgggcgtatagacccct

SEQ ID NO: 533 - DNA, 20 bp, synthetic construct
  source 1..20: mol_type = other DNA
taatgcgtggtgggtcccca

SEQ ID NO: 534 - DNA, 20 bp, synthetic construct
  source 1..20: mol_type = other DNA
aaggagggcttacccccagt

SEQ ID NO: 535 - DNA, 20 bp, synthetic construct
  source 1..20: mol_type = other DNA
catcatggggtgcgaggctt

SEQ ID NO: 536 - DNA, 20 bp, synthetic construct
  source 1..20: mol_type = other DNA
tctcgaagcctgtcttgaac

SEQ ID NO: 537 - DNA, 20 bp, synthetic construct
  source 1..20: mol_type = other DNA
ttgggagctggattgtagcg

SEQ ID NO: 538 - DNA, 20 bp, synthetic construct
  source 1..20: mol_type = other DNA
gggtctggtgagccgaatga

SEQ ID NO: 539 - DNA, 20 bp, synthetic construct
  source 1..20: mol_type = other DNA
agtccctatggctgcatcat

SEQ ID NO: 540 - DNA, 20 bp, synthetic construct
  source 1..20: mol_type = other DNA
tgggggtaagccctcctttt

SEQ ID NO: 541 - DNA, 20 bp, synthetic construct
  source 1..20: mol_type = other DNA
ttacccccagtagggcccat

SEQ ID NO: 542 - DNA, 20 bp, synthetic construct
  source 1..20: mol_type = other DNA
ttgagagggatgactcctat

SEQ ID NO: 543 - DNA, 20 bp, synthetic construct
  source 1..20: mol_type = other DNA
gggggtaagccctccttttt

SEQ ID NO: 544 - DNA, 20 bp, synthetic construct
  source 1..20: mol_type = other DNA
tgacaagctaatctttgcac

SEQ ID NO: 545 - DNA, 20 bp, synthetic construct
  source 1..20: mol_type = other DNA
gcccaatgcccttgtcattc

SEQ ID NO: 546 - DNA, 20 bp, synthetic construct
  source 1..20: mol_type = other DNA
tacccccagtagggcccatg

SEQ ID NO: 547 - DNA, 20 bp, synthetic construct
  source 1..20: mol_type = other DNA
cctagtcaaaggccatgcct

SEQ ID NO: 548 - DNA, 20 bp, synthetic construct
  source 1..20: mol_type = other DNA
cacagcggggcaatcagagc

SEQ ID NO: 549 - DNA, 20 bp, synthetic construct
  source 1..20: mol_type = other DNA
tagacccctgggctaccacc

SEQ ID NO: 550 - DNA, 20 bp, synthetic construct
  source 1..20: mol_type = other DNA
atcccgcccactgaaggagt

SEQ ID NO: 551 - DNA, 20 bp, synthetic construct
  source 1..20: mol_type = other DNA
tgcaggctgacagcatactc

SEQ ID NO: 552 - DNA, 20 bp, synthetic construct
  source 1..20: mol_type = other DNA
cagaaccaggtgatccccca

SEQ ID NO: 553 - DNA, 20 bp, synthetic construct
  source 1..20: mol_type = other DNA
gggtgtatccatctcactgt

SEQ ID NO: 554 - DNA, 20 bp, synthetic construct
  source 1..20: mol_type = other DNA
gggtccccatgggccctact

SEQ ID NO: 555 - DNA, 20 bp, synthetic construct
  source 1..20: mol_type = other DNA
caaggcatggcctttgacta

SEQ ID NO: 556 - DNA, 20 bp, synthetic construct
  source 1..20: mol_type = other DNA
gatctatctccaactctact

SEQ ID NO: 557 - DNA, 20 bp, synthetic construct
  source 1..20: mol_type = other DNA
cagtgagatggatacaccct

SEQ ID NO: 558 - DNA, 20 bp, synthetic construct
  source 1..20: mol_type = other DNA
gaagaaatcccgcccactga

SEQ ID NO: 559 - DNA, 20 bp, synthetic construct
  source 1..20: mol_type = other DNA
atcccccaggtggtagccca

SEQ ID NO: 560 - DNA, 20 bp, synthetic construct
  source 1..20: mol_type = other DNA
gtccccatgggccctactgg

SEQ ID NO: 561 - DNA, 20 bp, synthetic construct
  source 1..20: mol_type = other DNA
aatcccccaggtggtagccc

SEQ ID NO: 562 - DNA, 20 bp, synthetic construct
  source 1..20: mol_type = other DNA
catgagcctactccttcagt

SEQ ID NO: 563 - DNA, 20 bp, synthetic construct
  source 1..20: mol_type = other DNA
tcctagttccagttcaagac

SEQ ID NO: 564 - DNA, 20 bp, synthetic construct
  source 1..20: mol_type = other DNA
ggtccccatgggccctactg

SEQ ID NO: 565 - DNA, 20 bp, synthetic construct
  source 1..20: mol_type = other DNA
ccaccacgcattagtcccta

SEQ ID NO: 566 - DNA, 20 bp, synthetic construct
  source 1..20: mol_type = other DNA
tgggtggaccccattccaca

SEQ ID NO: 567 - DNA, 20 bp, synthetic construct
  source 1..20: mol_type = other DNA
agttagacatgccaatatgc

SEQ ID NO: 568 - DNA, 20 bp, synthetic construct
  source 1..20: mol_type = other DNA
agtgcaaagattagcttgtc

SEQ ID NO: 569 - DNA, 20 bp, synthetic construct
  source 1..20: mol_type = other DNA
tgggtccccatgggccctac

SEQ ID NO: 570 - DNA, 20 bp, synthetic construct
  source 1..20: mol_type = other DNA
atgcccttgtcattcaggtt

SEQ ID NO: 571 - DNA, 20 bp, synthetic construct
  source 1..20: mol_type = other DNA
tagtccctatggctgcatca

SEQ ID NO: 572 - DNA, 20 bp, synthetic construct
  source 1..20: mol_type = other DNA
gcctgtcttgaactggaact

SEQ ID NO: 573 - DNA, 20 bp, synthetic construct
  source 1..20: mol_type = other DNA
caaccatctgctgtcattgc

SEQ ID NO: 574 - DNA, 20 bp, synthetic construct
  source 1..20: mol_type = other DNA
acagtgagatggatacaccc

SEQ ID NO: 575 - DNA, 20 bp, synthetic construct
  source 1..20: mol_type = other DNA
tttgagagggatgactccta

SEQ ID NO: 576 - DNA, 20 bp, synthetic construct
  source 1..20: mol_type = other DNA
gccaaaagtggcttgtttgc

SEQ ID NO: 577 - DNA, 20 bp, synthetic construct
  source 1..20: mol_type = other DNA
gattgtagcgtggtgatggc

SEQ ID NO: 578 - DNA, 20 bp, synthetic construct
  source 1..20: mol_type = other DNA
ccactgaaggagtaggctca

SEQ ID NO: 579 - DNA, 20 bp, synthetic construct
  source 1..20: mol_type = other DNA
ggacaggttgcaggacacgc

SEQ ID NO: 580 - DNA, 20 bp, synthetic construct
  source 1..20: mol_type = other DNA
gcctgaagccatcctacccc

SEQ ID NO: 581 - DNA, 20 bp, synthetic construct
  source 1..20: mol_type = other DNA
tcactgtaggtagcacagcg

SEQ ID NO: 582 - DNA, 20 bp, synthetic construct
  source 1..20: mol_type = other DNA
ccaaggcatggcctttgact

SEQ ID NO: 583 - DNA, 20 bp, synthetic construct
  source 1..20: mol_type = other DNA
acctgaatgacaagggcatt

SEQ ID NO: 584 - DNA, 20 bp, synthetic construct
  source 1..20: mol_type = other DNA
ccatgagcctactccttcag

SEQ ID NO: 585 - DNA, 20 bp, synthetic construct
  source 1..20: mol_type = other DNA
aaacatggcagtgtctagac

SEQ ID NO: 586 - DNA, 20 bp, synthetic construct
  source 1..20: mol_type = other DNA
gggatgactcctatgggccc

SEQ ID NO: 587 - DNA, 20 bp, synthetic construct
  source 1..20: mol_type = other DNA
gcttccatgggttggagcat

SEQ ID NO: 588 - DNA, 20 bp, synthetic construct
  source 1..20: mol_type = other DNA
cttacccccagtagggccca

SEQ ID NO: 589 - DNA, 20 bp, synthetic construct
  source 1..20: mol_type = other DNA
gtgaactcagggctcccccc

SEQ ID NO: 590 - DNA, 20 bp, synthetic construct
  source 1..20: mol_type = other DNA
gtccctatggctgcatcatg

SEQ ID NO: 591 - DNA, 20 bp, synthetic construct
  source 1..20: mol_type = other DNA
ggcatggcctttgactaggg

SEQ ID NO: 592 - DNA, 20 bp, synthetic construct
  source 1..20: mol_type = other DNA
gcaccccatgatgcagccat

SEQ ID NO: 593 - DNA, 20 bp, synthetic construct
  source 1..20: mol_type = other DNA
atgagtgacggtgagcagac

SEQ ID NO: 594 - DNA, 20 bp, synthetic construct
  source 1..20: mol_type = other DNA
tggaagaatctcacctctcc

SEQ ID NO: 595 - DNA, 20 bp, synthetic construct
  source 1..20: mol_type = other DNA
ctgggtggaccccattccac

SEQ ID NO: 596 - DNA, 20 bp, synthetic construct
  source 1..20: mol_type = other DNA
tgacagcatactctggagat

SEQ ID NO: 597 - DNA, 20 bp, synthetic construct
  source 1..20: mol_type = other DNA
cagctctcctccctagtcaa

SEQ ID NO: 598 - DNA, 20 bp, synthetic construct
  source 1..20: mol_type = other DNA
ccagaaccaggtgatccccc

SEQ ID NO: 599 - DNA, 20 bp, synthetic construct
  source 1..20: mol_type = other DNA
agccaggtgatggaggcgcg

SEQ ID NO: 600 - DNA, 20 bp, synthetic construct
  source 1..20: mol_type = other DNA
cctttcctttttgcgtaagt

SEQ ID NO: 601 - DNA, 20 bp, synthetic construct
  source 1..20: mol_type = other DNA
tctggaaggcatctcaagga

SEQ ID NO: 602 - DNA, 20 bp, synthetic construct
  source 1..20: mol_type = other DNA
aacatggcagtgtctagaca

SEQ ID NO: 603 - DNA, 20 bp, synthetic construct
  source 1..20: mol_type = other DNA
tgaactcagggctcccccca

SEQ ID NO: 604 - DNA, 20 bp, synthetic construct
  source 1..20: mol_type = other DNA
agacccctgggctaccacct

SEQ ID NO: 605 - DNA, 20 bp, synthetic construct
  source 1..20: mol_type = other DNA
tcagccaatgctccaaccca

SEQ ID NO: 606 - RNA, 91 nt, synthetic construct
  source 1..91: mol_type = other RNA
catagggactaatgcgtggtgttttagagctagaaatagcaagttaaaataaggctagtccgttatcacgaaagggcaccgagtcggtgct

SEQ ID NO: 607 - RNA, 91 nt, synthetic construct
  source 1..91: mol_type = other RNA
caccgtcactcattcaagccgttttagagctagaaatagcaagttaaaataaggctagtccgttatcacgaaagggcaccgagtcggtgct

SEQ ID NO: 608 - RNA, 91 nt, synthetic construct
  source 1..91: mol_type = other RNA
gtggccaggttcagtcgaaggttttagagctagaaatagcaagttaaaataaggctagtccgttatcacgaaagggcaccgagtcggtgct

SEQ ID NO: 609 - RNA, 91 nt, synthetic construct
  source 1..91: mol_type = other RNA
aatgcgtggtgggtccccatgttttagagctagaaatagcaagttaaaataaggctagtccgttatcacgaaagggcaccgagtcggtgct

SEQ ID NO: 610 - RNA, 91 nt, synthetic construct
  source 1..91: mol_type = other RNA
agctcctcttcgactgaaccgttttagagctagaaatagcaagttaaaataaggctagtccgttatcacgaaagggcaccgagtcggtgct

SEQ ID NO: 611 - RNA, 91 nt, synthetic construct
  source 1..91: mol_type = other RNA
ccatagggactaatgcgtgggttttagagctagaaatagcaagttaaaataaggctagtccgttatcacgaaagggcaccgagtcggtgct

SEQ ID NO: 612 - RNA, 91 nt, synthetic construct
  source 1..91: mol_type = other RNA
cagccatagggactaatgcggttttagagctagaaatagcaagttaaaataaggctagtccgttatcacgaaagggcaccgagtcggtgct

SEQ ID NO: 613 - RNA, 91 nt, synthetic construct
  source 1..91: mol_type = other RNA
tcattcggctcaccagacccgttttagagctagaaatagcaagttaaaataaggctagtccgttatcacgaaagggcaccgagtcggtgct

SEQ ID NO: 614 - RNA, 91 nt, synthetic construct
  source 1..91: mol_type = other RNA
ggtctggtgagccgaatgaagttttagagctagaaatagcaagttaaaataaggctagtccgttatcacgaaagggcaccgagtcggtgct

SEQ ID NO: 615 - RNA, 91 nt, synthetic construct
  source 1..91: mol_type = other RNA
tggcttgtttgccggcatatgttttagagctagaaatagcaagttaaaataaggctagtccgttatcacgaaagggcaccgagtcggtgct

SEQ ID NO: 616 - RNA, 91 nt, synthetic construct
  source 1..91: mol_type = other RNA
aggagggcttacccccagtagttttagagctagaaatagcaagttaaaataaggctagtccgttatcacgaaagggcaccgagtcggtgct

SEQ ID NO: 617 - RNA, 91 nt, synthetic construct
  source 1..91: mol_type = other RNA
gccggcaaacaagccactttgttttagagctagaaatagcaagttaaaataaggctagtccgttatcacgaaagggcaccgagtcggtgct

SEQ ID NO: 618 - RNA, 91 nt, synthetic construct
  source 1..91: mol_type = other RNA
gagctgggcgtatagaccccgttttagagctagaaatagcaagttaaaataaggctagtccgttatcacgaaagggcaccgagtcggtgct

SEQ ID NO: 619 - RNA, 91 nt, synthetic construct
  source 1..91: mol_type = other RNA
gcatcatggggtgcgaggctgttttagagctagaaatagcaagttaaaataaggctagtccgttatcacgaaagggcaccgagtcggtgct

SEQ ID NO: 620 - RNA, 91 nt, synthetic construct
  source 1..91: mol_type = other RNA
agctgggcgtatagacccctgttttagagctagaaatagcaagttaaaataaggctagtccgttatcacgaaagggcaccgagtcggtgct

SEQ ID NO: 621 - RNA, 91 nt, synthetic construct
  source 1..91: mol_type = other RNA
taatgcgtggtgggtccccagttttagagctagaaatagcaagttaaaataaggctagtccgttatcacgaaagggcaccgagtcggtgct

SEQ ID NO: 622 - RNA, 91 nt, synthetic construct
  source 1..91: mol_type = other RNA
aaggagggcttacccccagtgttttagagctagaaatagcaagttaaaataaggctagtccgttatcacgaaagggcaccgagtcggtgct

SEQ ID NO: 623 - RNA, 91 nt, synthetic construct
  source 1..91: mol_type = other RNA
catcatggggtgcgaggcttgttttagagctagaaatagcaagttaaaataaggctagtccgttatcacgaaagggcaccgagtcggtgct

SEQ ID NO: 624 - RNA, 91 nt, synthetic construct
  source 1..91: mol_type = other RNA
tctcgaagcctgtcttgaacgttttagagctagaaatagcaagttaaaataaggctagtccgttatcacgaaagggcaccgagtcggtgct

SEQ ID NO: 625 - RNA, 91 nt, synthetic construct
  source 1..91: mol_type = other RNA
ttgggagctggattgtagcggttttagagctagaaatagcaagttaaaataaggctagtccgttatcacgaaagggcaccgagtcggtgct

SEQ ID NO: 626 - RNA, 91 nt, synthetic construct
  source 1..91: mol_type = other RNA
gggtctggtgagccgaatgagttttagagctagaaatagcaagttaaaataaggctagtccgttatcacgaaagggcaccgagtcggtgct

SEQ ID NO: 627 - RNA, 91 nt, synthetic construct
  source 1..91: mol_type = other RNA
agtccctatggctgcatcatgttttagagctagaaatagcaagttaaaataaggctagtccgttatcacgaaagggcaccgagtcggtgct

SEQ ID NO: 628 - RNA, 91 nt, synthetic construct
  source 1..91: mol_type = other RNA
tgggggtaagccctccttttgttttagagctagaaatagcaagttaaaataaggctagtccgttatcacgaaagggcaccgagtcggtgct

SEQ ID NO: 629 - RNA, 91 nt, synthetic construct
  source 1..91: mol_type = other RNA
ttacccccagtagggcccatgttttagagctagaaatagcaagttaaaataaggctagtccgttatcacgaaagggcaccgagtcggtgct

SEQ ID NO: 630 - RNA, 91 nt, synthetic construct
  source 1..91: mol_type = other RNA
ttgagagggatgactcctatgttttagagctagaaatagcaagttaaaataaggctagtccgttatcacgaaagggcaccgagtcggtgct

SEQ ID NO: 631 - RNA, 91 nt, synthetic construct
  source 1..91: mol_type = other RNA
gggggtaagccctcctttttgttttagagctagaaatagcaagttaaaataaggctagtccgttatcacgaaagggcaccgagtcggtgct

SEQ ID NO: 632 - RNA, 91 nt, synthetic construct
  source 1..91: mol_type = other RNA
tgacaagctaatctttgcacgttttagagctagaaatagcaagttaaaataaggctagtccgttatcacgaaagggcaccgagtcggtgct

SEQ ID NO: 633 - RNA, 91 nt, synthetic construct
  source 1..91: mol_type = other RNA
gcccaatgcccttgtcattcgttttagagctagaaatagcaagttaaaataaggctagtccgttatcacgaaagggcaccgagtcggtgct

SEQ ID NO: 634 - RNA, 91 nt, synthetic construct
  source 1..91: mol_type = other RNA
tacccccagtagggcccatggttttagagctagaaatagcaagttaaaataaggctagtccgttatcacgaaagggcaccgagtcggtgct

SEQ ID NO: 635 - RNA, 91 nt, synthetic construct
  source 1..91: mol_type = other RNA
cctagtcaaaggccatgcctgttttagagctagaaatagcaagttaaaataaggctagtccgttatcacgaaagggcaccgagtcggtgct

SEQ ID NO: 636 - RNA, 91 nt, synthetic construct
  source 1..91: mol_type = other RNA
cacagcggggcaatcagagcgttttagagctagaaatagcaagttaaaataaggctagtccgttatcacgaaagggcaccgagtcggtgct

SEQ ID NO: 637 - RNA, 91 nt, synthetic construct
  source 1..91: mol_type = other RNA
tagacccctgggctaccaccgttttagagctagaaatagcaagttaaaataaggctagtccgttatcacgaaagggcaccgagtcggtgct

SEQ ID NO: 638 - RNA, 91 nt, synthetic construct
  source 1..91: mol_type = other RNA
atcccgcccactgaaggagtgttttagagctagaaatagcaagttaaaataaggctagtccgttatcacgaaagggcaccgagtcggtgct

SEQ ID NO: 639 - RNA, 91 nt, synthetic construct
  source 1..91: mol_type = other RNA
tgcaggctgacagcatactcgttttagagctagaaatagcaagttaaaataaggctagtccgttatcacgaaagggcaccgagtcggtgct

SEQ ID NO: 640 - RNA, 91 nt, synthetic construct
  source 1..91: mol_type = other RNA
cagaaccaggtgatcccccagttttagagctagaaatagcaagttaaaataaggctagtccgttatcacgaaagggcaccgagtcggtgct

SEQ ID NO: 641 - RNA, 91 nt, synthetic construct
  source 1..91: mol_type = other RNA
gggtgtatccatctcactgtgttttagagctagaaatagcaagttaaaataaggctagtccgttatcacgaaagggcaccgagtcggtgct

SEQ ID NO: 642 - RNA, 91 nt, synthetic construct
  source 1..91: mol_type = other RNA
gggtccccatgggccctactgttttagagctagaaatagcaagttaaaataaggctagtccgttatcacgaaagggcaccgagtcggtgct

SEQ ID NO: 643 - RNA, 91 nt, synthetic construct
  source 1..91: mol_type = other RNA
caaggcatggcctttgactagttttagagctagaaatagcaagttaaaataaggctagtccgttatcacgaaagggcaccgagtcggtgct

SEQ ID NO: 644 - RNA, 91 nt, synthetic construct
  source 1..91: mol_type = other RNA
gatctatctccaactctactgttttagagctagaaatagcaagttaaaataaggctagtccgttatcacgaaagggcaccgagtcggtgct

SEQ ID NO: 645 - RNA, 91 nt, synthetic construct
  source 1..91: mol_type = other RNA
cagtgagatggatacaccctgttttagagctagaaatagcaagttaaaataaggctagtccgttatcacgaaagggcaccgagtcggtgct

SEQ ID NO: 646 - RNA, 91 nt, synthetic construct
  source 1..91: mol_type = other RNA
gaagaaatcccgcccactgagttttagagctagaaatagcaagttaaaataaggctagtccgttatcacgaaagggcaccgagtcggtgct

SEQ ID NO: 647 - RNA, 91 nt, synthetic construct
  source 1..91: mol_type = other RNA
atcccccaggtggtagcccagttttagagctagaaatagcaagttaaaataaggctagtccgttatcacgaaagggcaccgagtcggtgct

SEQ ID NO: 648 - RNA, 91 nt, synthetic construct
  source 1..91: mol_type = other RNA
gtccccatgggccctactgggttttagagctagaaatagcaagttaaaataaggctagtccgttatcacgaaagggcaccgagtcggtgct

SEQ ID NO: 649 - RNA, 91 nt, synthetic construct
  source 1..91: mol_type = other RNA
aatcccccaggtggtagcccgttttagagctagaaatagcaagttaaaataaggctagtccgttatcacgaaagggcaccgagtcggtgct

SEQ ID NO: 650 - RNA, 91 nt, synthetic construct
  source 1..91: mol_type = other RNA
catgagcctactccttcagtgttttagagctagaaatagcaagttaaaataaggctagtccgttatcacgaaagggcaccgagtcggtgct

SEQ ID NO: 651 - RNA, 91 nt, synthetic construct
  source 1..91: mol_type = other RNA
tcctagttccagttcaagacgttttagagctagaaatagcaagttaaaataaggctagtccgttatcacgaaagggcaccgagtcggtgct

SEQ ID NO: 652 - RNA, 91 nt, synthetic construct
  source 1..91: mol_type = other RNA
ggtccccatgggccctactggttttagagctagaaatagcaagttaaaataaggctagtccgttatcacgaaagggcaccgagtcggtgct

SEQ ID NO: 653 - RNA, 91 nt, synthetic construct
  source 1..91: mol_type = other RNA
ccaccacgcattagtccctagttttagagctagaaatagcaagttaaaataaggctagtccgttatcacgaaagggcaccgagtcggtgct

SEQ ID NO: 654 - RNA, 91 nt, synthetic construct
  source 1..91: mol_type = other RNA
tgggtggaccccattccacagttttagagctagaaatagcaagttaaaataaggctagtccgttatcacgaaagggcaccgagtcggtgct

SEQ ID NO: 655 - RNA, 91 nt, synthetic construct
  source 1..91: mol_type = other RNA
agttagacatgccaatatgcgttttagagctagaaatagcaagttaaaataaggctagtccgttatcacgaaagggcaccgagtcggtgct

SEQ ID NO: 656 - RNA, 91 nt, synthetic construct
  source 1..91: mol_type = other RNA
agtgcaaagattagcttgtcgttttagagctagaaatagcaagttaaaataaggctagtccgttatcacgaaagggcaccgagtcggtgct

SEQ ID NO: 657 - RNA, 91 nt, synthetic construct
  source 1..91: mol_type = other RNA
tgggtccccatgggccctacgttttagagctagaaatagcaagttaaaataaggctagtccgttatcacgaaagggcaccgagtcggtgct

SEQ ID NO: 658 - RNA, 91 nt, synthetic construct
  source 1..91: mol_type = other RNA
atgcccttgtcattcaggttgttttagagctagaaatagcaagttaaaataaggctagtccgttatcacgaaagggcaccgagtcggtgct

SEQ ID NO: 659 - RNA, 91 nt, synthetic construct
  source 1..91: mol_type = other RNA
tagtccctatggctgcatcagttttagagctagaaatagcaagttaaaataaggctagtccgttatcacgaaagggcaccgagtcggtgct

SEQ ID NO: 660 - RNA, 91 nt, synthetic construct
  source 1..91: mol_type = other RNA
gcctgtcttgaactggaactgttttagagctagaaatagcaagttaaaataaggctagtccgttatcacgaaagggcaccgagtcggtgct

SEQ ID NO: 661 - RNA, 91 nt, synthetic construct
  source 1..91: mol_type = other RNA
caaccatctgctgtcattgcgttttagagctagaaatagcaagttaaaataaggctagtccgttatcacgaaagggcaccgagtcggtgct

SEQ ID NO: 662 - RNA, 91 nt, synthetic construct
  source 1..91: mol_type = other RNA
acagtgagatggatacacccgttttagagctagaaatagcaagttaaaataaggctagtccgttatcacgaaagggcaccgagtcggtgct

SEQ ID NO: 663 - RNA, 91 nt, synthetic construct
  source 1..91: mol_type = other RNA
tttgagagggatgactcctagttttagagctagaaatagcaagttaaaataaggctagtccgttatcacgaaagggcaccgagtcggtgct

SEQ ID NO: 664 - RNA, 91 nt, synthetic construct
  source 1..91: mol_type = other RNA
gccaaaagtggcttgtttgcgttttagagctagaaatagcaagttaaaataaggctagtccgttatcacgaaagggcaccgagtcggtgct

SEQ ID NO: 665 - RNA, 91 nt, synthetic construct
  source 1..91: mol_type = other RNA
gattgtagcgtggtgatggcgttttagagctagaaatagcaagttaaaataaggctagtccgttatcacgaaagggcaccgagtcggtgct

SEQ ID NO: 666 - RNA, 91 nt, synthetic construct
  source 1..91: mol_type = other RNA
ccactgaaggagtaggctcagttttagagctagaaatagcaagttaaaataaggctagtccgttatcacgaaagggcaccgagtcggtgct

SEQ ID NO: 667 - RNA, 91 nt, synthetic construct
  source 1..91: mol_type = other RNA
ggacaggttgcaggacacgcgttttagagctagaaatagcaagttaaaataaggctagtccgttatcacgaaagggcaccgagtcggtgct

SEQ ID NO: 668 - RNA, 91 nt, synthetic construct
  source 1..91: mol_type = other RNA
gcctgaagccatcctaccccgttttagagctagaaatagcaagttaaaataaggctagtccgttatcacgaaagggcaccgagtcggtgct

SEQ ID NO: 669 - RNA, 91 nt, synthetic construct
  source 1..91: mol_type = other RNA
tcactgtaggtagcacagcggttttagagctagaaatagcaagttaaaataaggctagtccgttatcacgaaagggcaccgagtcggtgct

SEQ ID NO: 670 - RNA, 91 nt, synthetic construct
  source 1..91: mol_type = other RNA
ccaaggcatggcctttgactgttttagagctagaaatagcaagttaaaataaggctagtccgttatcacgaaagggcaccgagtcggtgct

SEQ ID NO: 671 - RNA, 91 nt, synthetic construct
  source 1..91: mol_type = other RNA
acctgaatgacaagggcattgttttagagctagaaatagcaagttaaaataaggctagtccgttatcacgaaagggcaccgagtcggtgct

SEQ ID NO: 672 - RNA, 91 nt, synthetic construct
  source 1..91: mol_type = other RNA
ccatgagcctactccttcaggttttagagctagaaatagcaagttaaaataaggctagtccgttatcacgaaagggcaccgagtcggtgct

SEQ ID NO: 673 - RNA, 91 nt, synthetic construct
  source 1..91: mol_type = other RNA
aaacatggcagtgtctagacgttttagagctagaaatagcaagttaaaataaggctagtccgttatcacgaaagggcaccgagtcggtgct

SEQ ID NO: 674 - RNA, 91 nt, synthetic construct
  source 1..91: mol_type = other RNA
gggatgactcctatgggcccgttttagagctagaaatagcaagttaaaataaggctagtccgttatcacgaaagggcaccgagtcggtgct

SEQ ID NO: 675 - RNA, 91 nt, synthetic construct
  source 1..91: mol_type = other RNA
gcttccatgggttggagcatgttttagagctagaaatagcaagttaaaataaggctagtccgttatcacgaaagggcaccgagtcggtgct

SEQ ID NO: 676 - RNA, 91 nt, synthetic construct
  source 1..91: mol_type = other RNA
cttacccccagtagggcccagttttagagctagaaatagcaagttaaaataaggctagtccgttatcacgaaagggcaccgagtcggtgct

SEQ ID NO: 677 - RNA, 91 nt, synthetic construct
  source 1..91: mol_type = other RNA
gtgaactcagggctccccccgttttagagctagaaatagcaagttaaaataaggctagtccgttatcacgaaagggcaccgagtcggtgct

SEQ ID NO: 678 - RNA, 91 nt, synthetic construct
  source 1..91: mol_type = other RNA
gtccctatggctgcatcatggttttagagctagaaatagcaagttaaaataaggctagtccgttatcacgaaagggcaccgagtcggtgct

SEQ ID NO: 679 - RNA, 91 nt, synthetic construct
  source 1..91: mol_type = other RNA
ggcatggcctttgactaggggttttagagctagaaatagcaagttaaaataaggctagtccgttatcacgaaagggcaccgagtcggtgct

SEQ ID NO: 680 - RNA, 91 nt, synthetic construct
  source 1..91: mol_type = other RNA
gcaccccatgatgcagccatgttttagagctagaaatagcaagttaaaataaggctagtccgttatcacgaaagggcaccgagtcggtgct

SEQ ID NO: 681 - RNA, 91 nt, synthetic construct
  source 1..91: mol_type = other RNA
atgagtgacggtgagcagacgttttagagctagaaatagcaagttaaaataaggctagtccgttatcacgaaagggcaccgagtcggtgct

SEQ ID NO: 682 - RNA, 91 nt, synthetic construct
  source 1..91: mol_type = other RNA
tggaagaatctcacctctccgttttagagctagaaatagcaagttaaaataaggctagtccgttatcacgaaagggcaccgagtcggtgct

SEQ ID NO: 683 - RNA, 91 nt, synthetic construct
  source 1..91: mol_type = other RNA
ctgggtggaccccattccacgttttagagctagaaatagcaagttaaaataaggctagtccgttatcacgaaagggcaccgagtcggtgct

SEQ ID NO: 684 - RNA, 91 nt, synthetic construct
  source 1..91: mol_type = other RNA
tgacagcatactctggagatgttttagagctagaaatagcaagttaaaataaggctagtccgttatcacgaaagggcaccgagtcggtgct

SEQ ID NO: 685 - RNA, 91 nt, synthetic construct
  source 1..91: mol_type = other RNA
cagctctcctccctagtcaagttttagagctagaaatagcaagttaaaataaggctagtccgttatcacgaaagggcaccgagtcggtgct

SEQ ID NO: 686 - RNA, 91 nt, synthetic construct
  source 1..91: mol_type = other RNA
ccagaaccaggtgatcccccgttttagagctagaaatagcaagttaaaataaggctagtccgttatcacgaaagggcaccgagtcggtgct

SEQ ID NO: 687 - RNA, 91 nt, synthetic construct
  source 1..91: mol_type = other RNA
agccaggtgatggaggcgcggttttagagctagaaatagcaagttaaaataaggctagtccgttatcacgaaagggcaccgagtcggtgct

SEQ ID NO: 688 - RNA, 91 nt, synthetic construct
  source 1..91: mol_type = other RNA
cctttcctttttgcgtaagtgttttagagctagaaatagcaagttaaaataaggctagtccgttatcacgaaagggcaccgagtcggtgct

SEQ ID NO: 689 - RNA, 91 nt, synthetic construct
  source 1..91: mol_type = other RNA
tctggaaggcatctcaaggagttttagagctagaaatagcaagttaaaataaggctagtccgttatcacgaaagggcaccgagtcggtgct

SEQ ID NO: 690 - RNA, 91 nt, synthetic construct
  source 1..91: mol_type = other RNA
aacatggcagtgtctagacagttttagagctagaaatagcaagttaaaataaggctagtccgttatcacgaaagggcaccgagtcggtgct

SEQ ID NO: 691 - RNA, 91 nt, synthetic construct
  source 1..91: mol_type = other RNA
tgaactcagggctccccccagttttagagctagaaatagcaagttaaaataaggctagtccgttatcacgaaagggcaccgagtcggtgct

SEQ ID NO: 692 - RNA, 91 nt, synthetic construct
  source 1..91: mol_type = other RNA
agacccctgggctaccacctgttttagagctagaaatagcaagttaaaataaggctagtccgttatcacgaaagggcaccgagtcggtgct

SEQ ID NO: 693 - RNA, 91 nt, synthetic construct
  source 1..91: mol_type = other RNA
tcagccaatgctccaacccagttttagagctagaaatagcaagttaaaataaggctagtccgttatcacgaaagggcaccgagtcggtgct

SEQ ID NO: 694 - DNA, 3341 bp, synthetic construct
  source 1..3341: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctaggtcagtgaagagaagaacaaaaagcagcatattacagttagttgtcttcatcaatctttaaatatgttgtgtggtttttctctccctgtttccacaggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaaggcggaggtgggtccgtgagcggctggcggctgttcaagaagattagctagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccggatccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgactagtcagctgatcttcttgaacagtctccagccagacacggacccacctccgcccttggctgtcaccttggactgcagtctgtggtactccttcagtctaaggctgttgatgttgatgaagcctgtggcgtcggtaggctcgtagtcgccctgaacattcatagacaccagctcctcattatagagggacagaggagactctctgcccaggatgtacacctgtcccttcagcacgctcacctgcactttgccttccactctctcctgggacttagcaatacagtgccggacgaactcgcactctggagaatgccagaatccggtgtacaccagctcggcgaatttcaggcccaggccttgcttgatcttccgcacctctctatccattgtgaaggcctcgatatccaggtgggcgtggtacaggatggttccggcaggggtttcgtagatgcctctgcttttcatgccgataaatctattttccacgatgtcgattcttcccacgccgtgcttgccagccacctcgttcaggtacatgaacagttccaggctggtctggtgggtagtgccgtccttcacgtttgtgaccttcacgggcactcccttcttaaactcgatttccaggatgtctggggtgttaggggccttggcggggtcctgggtcttggtatacaggccaggtggggcctggttcttagggttttccaggatgccagcttcgtagctgatgtgcatcagattctcatccatgctccaggggttcttgggggtcacagggatggggattccgtgctgcttggcgtattccatcaggtcgttcctgcccttgaagcggttgtagaattctggcatccgccaaggagcgatcaccttgatctgaggggccaggctgtagcagctcagctcgaatctgacctgatcgttgcctttgcctgtagcgccgtggctcacgtatttggcgccctctctctgggcgatctccacttgctttctggcgatgcaggggcgggccaggctggtgcccagcaggtaccggtcctcgtacagggcgctgctctggatagcaggccagataaattcttccacgaattcccgagacacatcttcaatgaacactttcttggcgcccagcttcagggctttctttctggcttcctcgaaatcctccttttgtccgatgttggcctgtggaaacagggagagaaaaaccacacaacatatttaaagattgatgaagacaactaactgtaatatgctgctttttgttcttctcttcactgacctagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 695 - DNA, 1131 bp, synthetic construct
  source 1..1131: mol_type = other DNA
gccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaa

SEQ ID NO: 696 - DNA, 1131 bp, synthetic construct
  source 1..1131: mol_type = other DNA
gccaacatcggacaaaaggaggatttcgaggaagccagaaagaaagccctgaagctgggcgccaagaaagtgttcattgaagatgtgtctcgggaattcgtggaagaatttatctggcctgctatccagagcagcgccctgtacgaggaccggtacctgctgggcaccagcctggcccgcccctgcatcgccagaaagcaagtggagatcgcccagagagagggcgccaaatacgtgagccacggcgctacaggcaaaggcaacgatcaggtcagattcgagctgagctgctacagcctggcccctcagatcaaggtgatcgctccttggcggatgccagaattctacaaccgcttcaagggcaggaacgacctgatggaatacgccaagcagcacggaatccccatccctgtgacccccaagaacccctggagcatggatgagaatctgatgcacatcagctacgaagctggcatcctggaaaaccctaagaaccaggccccacctggcctgtataccaagacccaggaccccgccaaggcccctaacaccccagacatcctggaaatcgagtttaagaagggagtgcccgtgaaggtcacaaacgtgaaggacggcactacccaccagaccagcctggaactgttcatgtacctgaacgaggtggctggcaagcacggcgtgggaagaatcgacatcgtggaaaatagatttatcggcatgaaaagcagaggcatctacgaaacccctgccggaaccatcctgtaccacgcccacctggatatcgaggccttcacaatggatagagaggtgcggaagatcaagcaaggcctgggcctgaaattcgccgagctggtgtacaccggattctggcattctccagagtgcgagttcgtccggcactgtattgctaagtcccaggagagagtggaaggcaaagtgcaggtgagcgtgctgaagggacaggtgtacatcctgggcagagagtctcctctgtccctctataatgaggagctggtgtctatgaatgttcagggcgactacgagcctaccgacgccacaggcttcatcaacatcaacagccttagactgaaggagtaccacagactgcagtccaaggtgacagccaag

SEQ ID NO: 697 - RNA, 91 nt, synthetic construct
  source 1..91: mol_type = other RNA
agacatcactaatcgagccggttttagagctagaaatagcaagttaaaataaggctagtccgttatcacgaaagggcaccgagtcggtgct

SEQ ID NO: 698 - RNA, 91 nt, synthetic construct
  source 1..91: mol_type = other RNA
accggtagggacccggcaacgttttagagctagaaatagcaagttaaaataaggctagtccgttatcacgaaagggcaccgagtcggtgct

SEQ ID NO: 699 - RNA, 91 nt, synthetic construct
  source 1..91: mol_type = other RNA
tgtggacttgggcccgttgcgttttagagctagaaatagcaagttaaaataaggctagtccgttatcacgaaagggcaccgagtcggtgct

SEQ ID NO: 700 - RNA, 91 nt, synthetic construct
  source 1..91: mol_type = other RNA
cctatgcagccgctcgtactgttttagagctagaaatagcaagttaaaataaggctagtccgttatcacgaaagggcaccgagtcggtgct

SEQ ID NO: 701 - RNA, 91 nt, synthetic construct
  source 1..91: mol_type = other RNA
gaatctgtagtcgtgatgtcgttttagagctagaaatagcaagttaaaataaggctagtccgttatcacgaaagggcaccgagtcggtgct

SEQ ID NO: 702 - RNA, 91 nt, synthetic construct
  source 1..91: mol_type = other RNA
caaaactgcatctggcgtccgttttagagctagaaatagcaagttaaaataaggctagtccgttatcacgaaagggcaccgagtcggtgct

SEQ ID NO: 703 - DNA, 3086 bp, synthetic construct
  source 1..3086: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctagatcaccggtggatcctaggtcagtgaagagaagaacaaaaagcagcatattacagttagttgtcttcatcaatctttaaatatgttgtgtggtttttctctccctgtttccacaggtgttcattgaggatgtcagcagggagtttgtggaggagttcatctggcctgccatccagtccagtgcactgtatgaggacagatacctcctgggcacctctcttgccaggccctgcattgccagaaaacaagtggaaattgcccagagggagggggccaagtatgtgtcccatggagccacaggaaaggggaatgatcaggtcaggtttgagctcagctgctactcactggccccccagataaaggtcattgctccctggaggatgcctgaattctacaacaggttcaagggcaggaatgacctgatggagtatgcaaagcaacatgggattcccatccctgtcactcccaagaacccctggagcatggatgagaacctcatgcacatcagctatgaggctggaatcctggagaaccccaagaaccaagcccctccaggtctctacaccaagacccaggacccagccaaagcccccaacacccctgacattctggagattgagttcaaaaaaggggtccctgtgaaggtgaccaatgtcaaggatggcaccacccaccagacctccttggagctcttcatgtacctgaatgaagtggctggcaagcatggagtgggcagaattgacattgtggagaacagattcattggaatgaagtccagaggcatctatgagaccccagcaggcaccatcctttaccatgctcatttagacattgaggccttcaccatggacagggaagtgaggaaaatcaaacaaggcctgggcttgaaatttgctgagctggtgtatacaggtttctggcacagccctgagtgtgaatttgtcaggcactgcattgccaagtcccaggagagagtggaagggaaagtgcaggtgtctgtcctcaagggccaggtgtacatcctgggcagggagtccccactgtctctctacaatgaggagctggtgagcatgaatgtgcagggtgattatgagccaactgatgccacagggttcatcaacatcaattccctcaggctgaaggaatatcatagactccagagcaaggtcactgccaaatagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttggggataccccctagagccccagctggttctttccacctcagaagccatagagcccactgcatccccagcatgcctgctattgtcttcccaatcctcccccttgctgtcctgccccaccccaccccccagaatagaatgacacctactcagacaatgtgatgcaatttcctcattttattaggaaaggacagtgggagtggcaccttccagggtcaaggaaggcaagggggaggggcaaacaacagatggctggcaactagaaggcacagtagaggtcacttggctgtcactttgctctgcagcctgtggtactctttcagcctcaggctgttgatgttgatgaatcctgtggcatctgtgggctcatagtccccttgcacattcatggacaccagttcctcattatacaggctcagagggctctctctgcccagaatgtacacctggcctttcagcacagacacttggaccttgccttccactctttcttgagacttggcaatacagtgcctgacaaattcacactcagggctgtgccaaaagcctgtgtacaccagctcagcaaacttcaggcccaggccttgcttgatctttctgacttctctgtccatggtgaaggcctcaatgtccaggtgggcatgatacaggatggtgccagctggtgtctcatagatgcccctggacttcatgccaatgaatctgttttccactatgtcaatcctgccaacaccatgtttgcctgccacctcattcaggtacatgaacagttccaggcttgtctggtgtgtggtgccatccttcacattggtcactttcactggcacccctttcttgaactcaatttccagaatgtcaggtgtgttgggagccttggctggatcctgtgtcttggtgtacaggccaggaggggcctgattcttagggttttccaggatgccagcctcatagctgatgtgcatcaggttctcatccatggaccagggattcttgggtgtcactggaatggggatgccatgctgcttggcatattccatcaggtcatttctgcccttgaatctgttgtagaactcaggcattctccaaggggcaatcactttgatttgaggggccagagagtagcagctcaactcaaatctgacttggtcattgcccttgcctgtggcaccatgggacacatatttggccccttctctctgggcaatttccacttgttttctggcaatgcaaggtctggccaggctggtgcccagcaggtatctgtcctcatacagggcagaactctggatggcaggccagatgaactcttccacaaattccctggacacatcctcaatgaacacctgtggaaacagggagagaaaaaccacacaacatatttaaagattgatgaagacaactaactgtaatatgctgctttttgttcttctcttcactgacctatctagagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 704 - DNA, 2798 bp, synthetic construct
  source 1..2798: mol_type = other DNA
taggtcagtgaagagaagaacaaaaagcagcatattacagttagttgtcttcatcaatctttaaatatgttgtgtggtttttctctccctgtttccacaggtgttcattgaggatgtcagcagggagtttgtggaggagttcatctggcctgccatccagtccagtgcactgtatgaggacagatacctcctgggcacctctcttgccaggccctgcattgccagaaaacaagtggaaattgcccagagggagggggccaagtatgtgtcccatggagccacaggaaaggggaatgatcaggtcaggtttgagctcagctgctactcactggccccccagataaaggtcattgctccctggaggatgcctgaattctacaacaggttcaagggcaggaatgacctgatggagtatgcaaagcaacatgggattcccatccctgtcactcccaagaacccctggagcatggatgagaacctcatgcacatcagctatgaggctggaatcctggagaaccccaagaaccaagcccctccaggtctctacaccaagacccaggacccagccaaagcccccaacacccctgacattctggagattgagttcaaaaaaggggtccctgtgaaggtgaccaatgtcaaggatggcaccacccaccagacctccttggagctcttcatgtacctgaatgaagtggctggcaagcatggagtgggcagaattgacattgtggagaacagattcattggaatgaagtccagaggcatctatgagaccccagcaggcaccatcctttaccatgctcatttagacattgaggccttcaccatggacagggaagtgaggaaaatcaaacaaggcctgggcttgaaatttgctgagctggtgtatacaggtttctggcacagccctgagtgtgaatttgtcaggcactgcattgccaagtcccaggagagagtggaagggaaagtgcaggtgtctgtcctcaagggccaggtgtacatcctgggcagggagtccccactgtctctctacaatgaggagctggtgagcatgaatgtgcagggtgattatgagccaactgatgccacagggttcatcaacatcaattccctcaggctgaaggaatatcatagactccagagcaaggtcactgccaaatagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttggggataccccctagagccccagctggttctttccacctcagaagccatagagcccactgcatccccagcatgcctgctattgtcttcccaatcctcccccttgctgtcctgccccaccccaccccccagaatagaatgacacctactcagacaatgtgatgcaatttcctcattttattaggaaaggacagtgggagtggcaccttccagggtcaaggaaggcaagggggaggggcaaacaacagatggctggcaactagaaggcacagtagaggtcacttggctgtcactttgctctgcagcctgtggtactctttcagcctcaggctgttgatgttgatgaatcctgtggcatctgtgggctcatagtccccttgcacattcatggacaccagttcctcattatacaggctcagagggctctctctgcccagaatgtacacctggcctttcagcacagacacttggaccttgccttccactctttcttgagacttggcaatacagtgcctgacaaattcacactcagggctgtgccaaaagcctgtgtacaccagctcagcaaacttcaggcccaggccttgcttgatctttctgacttctctgtccatggtgaaggcctcaatgtccaggtgggcatgatacaggatggtgccagctggtgtctcatagatgcccctggacttcatgccaatgaatctgttttccactatgtcaatcctgccaacaccatgtttgcctgccacctcattcaggtacatgaacagttccaggcttgtctggtgtgtggtgccatccttcacattggtcactttcactggcacccctttcttgaactcaatttccagaatgtcaggtgtgttgggagccttggctggatcctgtgtcttggtgtacaggccaggaggggcctgattcttagggttttccaggatgccagcctcatagctgatgtgcatcaggttctcatccatggaccagggattcttgggtgtcactggaatggggatgccatgctgcttggcatattccatcaggtcatttctgcccttgaatctgttgtagaactcaggcattctccaaggggcaatcactttgatttgaggggccagagagtagcagctcaactcaaatctgacttggtcattgcccttgcctgtggcaccatgggacacatatttggccccttctctctgggcaatttccacttgttttctggcaatgcaaggtctggccaggctggtgcccagcaggtatctgtcctcatacagggcagaactctggatggcaggccagatgaactcttccacaaattccctggacacatcctcaatgaacacctgtggaaacagggagagaaaaaccacacaacatatttaaagattgatgaagacaactaactgtaatatgctgctttttgttcttctcttcactgaccta

SEQ ID NO: 705 - DNA, 1062 bp, synthetic construct
  source 1..1062: mol_type = other DNA
gtgttcattgaggatgtcagcagggagtttgtggaggagttcatctggcctgccatccagtccagtgcactgtatgaggacagatacctcctgggcacctctcttgccaggccctgcattgccagaaaacaagtggaaattgcccagagggagggggccaagtatgtgtcccatggagccacaggaaaggggaatgatcaggtcaggtttgagctcagctgctactcactggccccccagataaaggtcattgctccctggaggatgcctgaattctacaacaggttcaagggcaggaatgacctgatggagtatgcaaagcaacatgggattcccatccctgtcactcccaagaacccctggagcatggatgagaacctcatgcacatcagctatgaggctggaatcctggagaaccccaagaaccaagcccctccaggtctctacaccaagacccaggacccagccaaagcccccaacacccctgacattctggagattgagttcaaaaaaggggtccctgtgaaggtgaccaatgtcaaggatggcaccacccaccagacctccttggagctcttcatgtacctgaatgaagtggctggcaagcatggagtgggcagaattgacattgtggagaacagattcattggaatgaagtccagaggcatctatgagaccccagcaggcaccatcctttaccatgctcatttagacattgaggccttcaccatggacagggaagtgaggaaaatcaaacaaggcctgggcttgaaatttgctgagctggtgtatacaggtttctggcacagccctgagtgtgaatttgtcaggcactgcattgccaagtcccaggagagagtggaagggaaagtgcaggtgtctgtcctcaagggccaggtgtacatcctgggcagggagtccccactgtctctctacaatgaggagctggtgagcatgaatgtgcagggtgattatgagccaactgatgccacagggttcatcaacatcaattccctcaggctgaaggaatatcatagactccagagcaaggtcactgccaaa

SEQ ID NO: 706 - DNA, 1062 bp, synthetic construct
  source 1..1062: mol_type = other DNA
gtgttcattgaggatgtgtccagggaatttgtggaagagttcatctggcctgccatccagagttctgccctgtatgaggacagatacctgctgggcaccagcctggccagaccttgcattgccagaaaacaagtggaaattgcccagagagaaggggccaaatatgtgtcccatggtgccacaggcaagggcaatgaccaagtcagatttgagttgagctgctactctctggcccctcaaatcaaagtgattgccccttggagaatgcctgagttctacaacagattcaagggcagaaatgacctgatggaatatgccaagcagcatggcatccccattccagtgacacccaagaatccctggtccatggatgagaacctgatgcacatcagctatgaggctggcatcctggaaaaccctaagaatcaggcccctcctggcctgtacaccaagacacaggatccagccaaggctcccaacacacctgacattctggaaattgagttcaagaaaggggtgccagtgaaagtgaccaatgtgaaggatggcaccacacaccagacaagcctggaactgttcatgtacctgaatgaggtggcaggcaaacatggtgttggcaggattgacatagtggaaaacagattcattggcatgaagtccaggggcatctatgagacaccagctggcaccatcctgtatcatgcccacctggacattgaggccttcaccatggacagagaagtcagaaagatcaagcaaggcctgggcctgaagtttgctgagctggtgtacacaggcttttggcacagccctgagtgtgaatttgtcaggcactgtattgccaagtctcaagaaagagtggaaggcaaggtccaagtgtctgtgctgaaaggccaggtgtacattctgggcagagagagccctctgagcctgtataatgaggaactggtgtccatgaatgtgcaaggggactatgagcccacagatgccacaggattcatcaacatcaacagcctgaggctgaaagagtaccacaggctgcagagcaaagtgacagccaag

SEQ ID NO: 707 - DNA, 3086 bp, synthetic construct
  source 1..3086: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctagatcaccggtggatcctaggtcagtgaagagaagaacaaaaagcagcatattacagttagttgtcttcatcaatctttaaatatgttgtgtggtttttctctccctgtttccacaggtgttcattgaggatgtcagcagggagtttgtggaggagttcatctggcctgccatccagtccagtgcactgtatgaggacagatacctcctgggcacctctcttgccaggccctgcattgccagaaaacaagtggaaattgcccagagggagggggccaagtatgtgtcccatggtgccacaggaaaggggaatgatcaggtcaggtttgagctcagctgctactcactggccccccagataaaggtcattgctccctggaggatgcctgaattctacaacaggttcaagggcagaaatgacctgatggagtatgcaaagcaacatgggattcccatcccagtcactcccaagaacccctggagcatggatgagaacctcatgcacatcagctatgaggctggaatcctggagaaccccaagaaccaagcccctcctggtctctacaccaagacccaggacccagccaaagcccccaacacccctgacattctggagattgagttcaaaaaaggggtccctgtgaaggtgaccaatgtcaaggatggcaccacccaccagacctccttggagctcttcatgtacctgaatgaagtggcaggcaagcatggtgtgggcaggattgacatagtggagaacagattcattggaatgaagtccagaggcatctatgagaccccagcaggcaccatcctttaccatgctcatttagacattgaggccttcaccatggacagggaagtgagaaaaatcaaacaaggcctgggcttgaaatttgctgagctggtgtatacaggtttctggcacagccctgagtgtgaatttgtcaggcactgcattgccaagtcccaggagagagtggaagggaaagtgcaggtgtctgtcctcaagggccaggtgtacatcctgggcagggagtccccactgtctctctacaatgaggagctggtgagcatgaatgtgcagggtgattatgagccaactgatgccacagggttcatcaacatcaattccctgaggctgaaggaatatcataggctccagagcaaggtcactgccaaatagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttggggataccccctagagccccagctggttctttccacctcagaagccatagagcccactgcatccccagcatgcctgctattgtcttcccaatcctcccccttgctgtcctgccccaccccaccccccagaatagaatgacacctactcagacaatgtgatgcaatttcctcattttattaggaaaggacagtgggagtggcaccttccagggtcaaggaaggcaagggggaggggcaaacaacagatggctggcaactagaaggcacagtagaggtcacttggctgtcaccttggactgcagtctgtggtactccttcagtctaaggctgttgatgttgatgaagcctgtggcatctgtaggctcatagtctccctgaacattcatagacaccagctcctcattatagagggacagaggagactctctgcccaggatgtacacctgtcccttcagcacagacacctgcactttgccttccactctctcctgggacttagcaatacagtgcctgacaaactcacactcaggagaatgccagaatcctgtgtacaccagctcagcaaatttcaggcccaggccttgcttgatcttcctgacttctctatccattgtgaaggcctcaatatccaggtgggcatggtacaggatggttccagcaggggtttcatagatgcctctgcttttcatgccaataaatctattttccacaatgtcaattcttcccactccatgcttgccagccacctcattcaggtacatgaacagttccaggctggtctggtgggtagtgccatccttcacatttgtgaccttcacaggcactcccttcttaaactcaatttccaggatgtctggggtgttaggggccttggcagggtcctgggtcttggtatacaggccaggtggggcctggttcttagggttttccaggatgccagcttcatagctgatgtgcatcagattctcatccatgctccaggggttcttgggggtcacagggatggggattccatgctgcttggcatattccatcaggtcattcctgcccttgaacctgttgtagaattctggcatcctccaaggagcaatcaccttgatttgaggggccaggctgtagcagctcagctcaaacctgacttgatcattccctttgcctgtagctccatggctcacatatttggctccctctctctgggcaatctccacttgctttctggcaatgcagggcctggccaggctggtgcccagcaggtacctgtcctcatacagggcagagctctggatagcaggccagataaattcttccacaaattccctagacacatcttcaatgaacacctgtggaaacagggagagaaaaaccacacaacatatttaaagattgatgaagacaactaactgtaatatgctgctttttgttcttctcttcactgacctatctagagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 708 - DNA, 2798 bp, synthetic construct
  source 1..2798: mol_type = other DNA
taggtcagtgaagagaagaacaaaaagcagcatattacagttagttgtcttcatcaatctttaaatatgttgtgtggtttttctctccctgtttccacaggtgttcattgaggatgtcagcagggagtttgtggaggagttcatctggcctgccatccagtccagtgcactgtatgaggacagatacctcctgggcacctctcttgccaggccctgcattgccagaaaacaagtggaaattgcccagagggagggggccaagtatgtgtcccatggtgccacaggaaaggggaatgatcaggtcaggtttgagctcagctgctactcactggccccccagataaaggtcattgctccctggaggatgcctgaattctacaacaggttcaagggcagaaatgacctgatggagtatgcaaagcaacatgggattcccatcccagtcactcccaagaacccctggagcatggatgagaacctcatgcacatcagctatgaggctggaatcctggagaaccccaagaaccaagcccctcctggtctctacaccaagacccaggacccagccaaagcccccaacacccctgacattctggagattgagttcaaaaaaggggtccctgtgaaggtgaccaatgtcaaggatggcaccacccaccagacctccttggagctcttcatgtacctgaatgaagtggcaggcaagcatggtgtgggcaggattgacatagtggagaacagattcattggaatgaagtccagaggcatctatgagaccccagcaggcaccatcctttaccatgctcatttagacattgaggccttcaccatggacagggaagtgagaaaaatcaaacaaggcctgggcttgaaatttgctgagctggtgtatacaggtttctggcacagccctgagtgtgaatttgtcaggcactgcattgccaagtcccaggagagagtggaagggaaagtgcaggtgtctgtcctcaagggccaggtgtacatcctgggcagggagtccccactgtctctctacaatgaggagctggtgagcatgaatgtgcagggtgattatgagccaactgatgccacagggttcatcaacatcaattccctgaggctgaaggaatatcataggctccagagcaaggtcactgccaaatagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttggggataccccctagagccccagctggttctttccacctcagaagccatagagcccactgcatccccagcatgcctgctattgtcttcccaatcctcccccttgctgtcctgccccaccccaccccccagaatagaatgacacctactcagacaatgtgatgcaatttcctcattttattaggaaaggacagtgggagtggcaccttccagggtcaaggaaggcaagggggaggggcaaacaacagatggctggcaactagaaggcacagtagaggtcacttggctgtcaccttggactgcagtctgtggtactccttcagtctaaggctgttgatgttgatgaagcctgtggcatctgtaggctcatagtctccctgaacattcatagacaccagctcctcattatagagggacagaggagactctctgcccaggatgtacacctgtcccttcagcacagacacctgcactttgccttccactctctcctgggacttagcaatacagtgcctgacaaactcacactcaggagaatgccagaatcctgtgtacaccagctcagcaaatttcaggcccaggccttgcttgatcttcctgacttctctatccattgtgaaggcctcaatatccaggtgggcatggtacaggatggttccagcaggggtttcatagatgcctctgcttttcatgccaataaatctattttccacaatgtcaattcttcccactccatgcttgccagccacctcattcaggtacatgaacagttccaggctggtctggtgggtagtgccatccttcacatttgtgaccttcacaggcactcccttcttaaactcaatttccaggatgtctggggtgttaggggccttggcagggtcctgggtcttggtatacaggccaggtggggcctggttcttagggttttccaggatgccagcttcatagctgatgtgcatcagattctcatccatgctccaggggttcttgggggtcacagggatggggattccatgctgcttggcatattccatcaggtcattcctgcccttgaacctgttgtagaattctggcatcctccaaggagcaatcaccttgatttgaggggccaggctgtagcagctcagctcaaacctgacttgatcattccctttgcctgtagctccatggctcacatatttggctccctctctctgggcaatctccacttgctttctggcaatgcagggcctggccaggctggtgcccagcaggtacctgtcctcatacagggcagagctctggatagcaggccagataaattcttccacaaattccctagacacatcttcaatgaacacctgtggaaacagggagagaaaaaccacacaacatatttaaagattgatgaagacaactaactgtaatatgctgctttttgttcttctcttcactgaccta

SEQ ID NO: 709 - DNA, 1062 bp, synthetic construct
  source 1..1062: mol_type = other DNA
gtgttcattgaggatgtcagcagggagtttgtggaggagttcatctggcctgccatccagtccagtgcactgtatgaggacagatacctcctgggcacctctcttgccaggccctgcattgccagaaaacaagtggaaattgcccagagggagggggccaagtatgtgtcccatggtgccacaggaaaggggaatgatcaggtcaggtttgagctcagctgctactcactggccccccagataaaggtcattgctccctggaggatgcctgaattctacaacaggttcaagggcagaaatgacctgatggagtatgcaaagcaacatgggattcccatcccagtcactcccaagaacccctggagcatggatgagaacctcatgcacatcagctatgaggctggaatcctggagaaccccaagaaccaagcccctcctggtctctacaccaagacccaggacccagccaaagcccccaacacccctgacattctggagattgagttcaaaaaaggggtccctgtgaaggtgaccaatgtcaaggatggcaccacccaccagacctccttggagctcttcatgtacctgaatgaagtggcaggcaagcatggtgtgggcaggattgacatagtggagaacagattcattggaatgaagtccagaggcatctatgagaccccagcaggcaccatcctttaccatgctcatttagacattgaggccttcaccatggacagggaagtgagaaaaatcaaacaaggcctgggcttgaaatttgctgagctggtgtatacaggtttctggcacagccctgagtgtgaatttgtcaggcactgcattgccaagtcccaggagagagtggaagggaaagtgcaggtgtctgtcctcaagggccaggtgtacatcctgggcagggagtccccactgtctctctacaatgaggagctggtgagcatgaatgtgcagggtgattatgagccaactgatgccacagggttcatcaacatcaattccctgaggctgaaggaatatcataggctccagagcaaggtcactgccaaa

SEQ ID NO: 710 - DNA, 1062 bp, synthetic construct
  source 1..1062: mol_type = other DNA
gtgttcattgaagatgtgtctagggaatttgtggaagaatttatctggcctgctatccagagctctgccctgtatgaggacaggtacctgctgggcaccagcctggccaggccctgcattgccagaaagcaagtggagattgcccagagagagggagccaaatatgtgagccatggagctacaggcaaagggaatgatcaagtcaggtttgagctgagctgctacagcctggcccctcaaatcaaggtgattgctccttggaggatgccagaattctacaacaggttcaagggcaggaatgacctgatggaatatgccaagcagcatggaatccccatccctgtgacccccaagaacccctggagcatggatgagaatctgatgcacatcagctatgaagctggcatcctggaaaaccctaagaaccaggccccacctggcctgtataccaagacccaggaccctgccaaggcccctaacaccccagacatcctggaaattgagtttaagaagggagtgcctgtgaaggtcacaaatgtgaaggatggcactacccaccagaccagcctggaactgttcatgtacctgaatgaggtggctggcaagcatggagtgggaagaattgacattgtggaaaatagatttattggcatgaaaagcagaggcatctatgaaacccctgctggaaccatcctgtaccatgcccacctggatattgaggccttcacaatggatagagaagtcaggaagatcaagcaaggcctgggcctgaaatttgctgagctggtgtacacaggattctggcattctcctgagtgtgagtttgtcaggcactgtattgctaagtcccaggagagagtggaaggcaaagtgcaggtgtctgtgctgaagggacaggtgtacatcctgggcagagagtctcctctgtccctctataatgaggagctggtgtctatgaatgttcagggagactatgagcctacagatgccacaggcttcatcaacatcaacagccttagactgaaggagtaccacagactgcagtccaaggtgacagccaag

SEQ ID NO: 711 - DNA, 3086 bp, synthetic construct
  source 1..3086: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctagatcaccggtggatcctaggtcagtgaagagaagaacaaaaagcagcatattacagttagttgtcttcatcaatctttaaatatgttgtgtggtttttctctccctgtttccacaggtgttcattgaggatgtcagcagggagtttgtggaggagttcatctggcctgccatccagtccagtgcactgtatgaggacagatacctcctgggcacctctcttgccaggccctgcattgccagaaaacaagtggaaattgcccagagggagggggccaagtatgtgtcccatggtgccacaggaaaggggaatgatcaggtcaggtttgagctcagctgctactcactggccccccagataaaggtcattgctccctggaggatgcctgaattctacaacaggttcaagggcagaaatgacctgatggagtatgcaaagcaacatgggattcccatcccagtcactcccaagaacccctggagcatggatgagaacctcatgcacatcagctatgaggctggaatcctggagaaccccaagaaccaagcccctccaggtctctacaccaagacccaggacccagccaaagcccccaacacccctgacattctggagattgagttcaaaaaaggggtccctgtgaaggtgaccaatgtcaaggatggcaccacccaccagacctccttggagctcttcatgtacctgaatgaagtggcaggcaagcatggtgtgggcaggattgacatagtggagaacagattcattggaatgaagtccagaggcatctatgagaccccagcaggcaccatcctttaccatgctcatttagacattgaggccttcaccatggacagggaagtgagaaaaatcaaacaaggcctgggcttgaaatttgctgagctggtgtatacaggtttctggcacagccctgagtgtgaatttgtcaggcactgcattgccaagtcccaggagagagtggaagggaaagtgcaggtgtctgtcctcaagggccaggtgtacatcctgggcagggagtccccactgtctctctacaatgaggagctggtgagcatgaatgtgcagggtgattatgagccaactgatgccacagggttcatcaacatcaattccctcaggctgaaggaatatcataggctccagagcaaggtcactgccaaatagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttggggataccccctagagccccagctggttctttccacctcagaagccatagagcccactgcatccccagcatgcctgctattgtcttcccaatcctcccccttgctgtcctgccccaccccaccccccagaatagaatgacacctactcagacaatgtgatgcaatttcctcattttattaggaaaggacagtgggagtggcaccttccagggtcaaggaaggcaagggggaggggcaaacaacagatggctggcaactagaaggcacagtagaggtcacttggctgtcactttgctctgcagcctgtggtactctttcagcctcaggctgttgatgttgatgaatcctgtggcatctgtgggctcatagtccccttgcacattcatggacaccagttcctcattatacaggctcagagggctctctctgcccagaatgtacacctggcctttcagcacagacacttggaccttgccttccactctttcttgagacttggcaatacagtgcctgacaaattcacactcagggctgtgccaaaagcctgtgtacaccagctcagcaaacttcaggcccaggccttgcttgatctttctgacttctctgtccatggtgaaggcctcaatgtccaggtgggcatgatacaggatggtgccagctggtgtctcatagatgcccctggacttcatgccaatgaatctgttttccactatgtcaatcctgccaacaccatgtttgcctgccacctcattcaggtacatgaacagttccaggcttgtctggtgtgtggtgccatccttcacattggtcactttcactggcacccctttcttgaactcaatttccagaatgtcaggtgtgttgggagccttggctggatcctgtgtcttggtgtacaggccaggaggggcctgattcttagggttttccaggatgccagcctcatagctgatgtgcatcaggttctcatccatggaccagggattcttgggtgtcactggaatggggatgccatgctgcttggcatattccatcaggtcatttctgcccttgaatctgttgtagaactcaggcattctccaaggggcaatcactttgatttgaggggccagagagtagcagctcaactcaaatctgacttggtcattgcccttgcctgtggcaccatgggacacatatttggccccttctctctgggcaatttccacttgttttctggcaatgcaaggtctggccaggctggtgcccagcaggtatctgtcctcatacagggcagaactctggatggcaggccagatgaactcttccacaaattccctggacacatcctcaatgaacacctgtggaaacagggagagaaaaaccacacaacatatttaaagattgatgaagacaactaactgtaatatgctgctttttgttcttctcttcactgacctatctagagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 712 - DNA, 2798 bp, synthetic construct
  source 1..2798: mol_type = other DNA
taggtcagtgaagagaagaacaaaaagcagcatattacagttagttgtcttcatcaatctttaaatatgttgtgtggtttttctctccctgtttccacaggtgttcattgaggatgtcagcagggagtttgtggaggagttcatctggcctgccatccagtccagtgcactgtatgaggacagatacctcctgggcacctctcttgccaggccctgcattgccagaaaacaagtggaaattgcccagagggagggggccaagtatgtgtcccatggtgccacaggaaaggggaatgatcaggtcaggtttgagctcagctgctactcactggccccccagataaaggtcattgctccctggaggatgcctgaattctacaacaggttcaagggcagaaatgacctgatggagtatgcaaagcaacatgggattcccatcccagtcactcccaagaacccctggagcatggatgagaacctcatgcacatcagctatgaggctggaatcctggagaaccccaagaaccaagcccctccaggtctctacaccaagacccaggacccagccaaagcccccaacacccctgacattctggagattgagttcaaaaaaggggtccctgtgaaggtgaccaatgtcaaggatggcaccacccaccagacctccttggagctcttcatgtacctgaatgaagtggcaggcaagcatggtgtgggcaggattgacatagtggagaacagattcattggaatgaagtccagaggcatctatgagaccccagcaggcaccatcctttaccatgctcatttagacattgaggccttcaccatggacagggaagtgagaaaaatcaaacaaggcctgggcttgaaatttgctgagctggtgtatacaggtttctggcacagccctgagtgtgaatttgtcaggcactgcattgccaagtcccaggagagagtggaagggaaagtgcaggtgtctgtcctcaagggccaggtgtacatcctgggcagggagtccccactgtctctctacaatgaggagctggtgagcatgaatgtgcagggtgattatgagccaactgatgccacagggttcatcaacatcaattccctcaggctgaaggaatatcataggctccagagcaaggtcactgccaaatagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttggggataccccctagagccccagctggttctttccacctcagaagccatagagcccactgcatccccagcatgcctgctattgtcttcccaatcctcccccttgctgtcctgccccaccccaccccccagaatagaatgacacctactcagacaatgtgatgcaatttcctcattttattaggaaaggacagtgggagtggcaccttccagggtcaaggaaggcaagggggaggggcaaacaacagatggctggcaactagaaggcacagtagaggtcacttggctgtcactttgctctgcagcctgtggtactctttcagcctcaggctgttgatgttgatgaatcctgtggcatctgtgggctcatagtccccttgcacattcatggacaccagttcctcattatacaggctcagagggctctctctgcccagaatgtacacctggcctttcagcacagacacttggaccttgccttccactctttcttgagacttggcaatacagtgcctgacaaattcacactcagggctgtgccaaaagcctgtgtacaccagctcagcaaacttcaggcccaggccttgcttgatctttctgacttctctgtccatggtgaaggcctcaatgtccaggtgggcatgatacaggatggtgccagctggtgtctcatagatgcccctggacttcatgccaatgaatctgttttccactatgtcaatcctgccaacaccatgtttgcctgccacctcattcaggtacatgaacagttccaggcttgtctggtgtgtggtgccatccttcacattggtcactttcactggcacccctttcttgaactcaatttccagaatgtcaggtgtgttgggagccttggctggatcctgtgtcttggtgtacaggccaggaggggcctgattcttagggttttccaggatgccagcctcatagctgatgtgcatcaggttctcatccatggaccagggattcttgggtgtcactggaatggggatgccatgctgcttggcatattccatcaggtcatttctgcccttgaatctgttgtagaactcaggcattctccaaggggcaatcactttgatttgaggggccagagagtagcagctcaactcaaatctgacttggtcattgcccttgcctgtggcaccatgggacacatatttggccccttctctctgggcaatttccacttgttttctggcaatgcaaggtctggccaggctggtgcccagcaggtatctgtcctcatacagggcagaactctggatggcaggccagatgaactcttccacaaattccctggacacatcctcaatgaacacctgtggaaacagggagagaaaaaccacacaacatatttaaagattgatgaagacaactaactgtaatatgctgctttttgttcttctcttcactgaccta

SEQ ID NO: 713 - DNA, 1062 bp, synthetic construct
  source 1..1062: mol_type = other DNA
gtgttcattgaggatgtcagcagggagtttgtggaggagttcatctggcctgccatccagtccagtgcactgtatgaggacagatacctcctgggcacctctcttgccaggccctgcattgccagaaaacaagtggaaattgcccagagggagggggccaagtatgtgtcccatggtgccacaggaaaggggaatgatcaggtcaggtttgagctcagctgctactcactggccccccagataaaggtcattgctccctggaggatgcctgaattctacaacaggttcaagggcagaaatgacctgatggagtatgcaaagcaacatgggattcccatcccagtcactcccaagaacccctggagcatggatgagaacctcatgcacatcagctatgaggctggaatcctggagaaccccaagaaccaagcccctccaggtctctacaccaagacccaggacccagccaaagcccccaacacccctgacattctggagattgagttcaaaaaaggggtccctgtgaaggtgaccaatgtcaaggatggcaccacccaccagacctccttggagctcttcatgtacctgaatgaagtggcaggcaagcatggtgtgggcaggattgacatagtggagaacagattcattggaatgaagtccagaggcatctatgagaccccagcaggcaccatcctttaccatgctcatttagacattgaggccttcaccatggacagggaagtgagaaaaatcaaacaaggcctgggcttgaaatttgctgagctggtgtatacaggtttctggcacagccctgagtgtgaatttgtcaggcactgcattgccaagtcccaggagagagtggaagggaaagtgcaggtgtctgtcctcaagggccaggtgtacatcctgggcagggagtccccactgtctctctacaatgaggagctggtgagcatgaatgtgcagggtgattatgagccaactgatgccacagggttcatcaacatcaattccctcaggctgaaggaatatcataggctccagagcaaggtcactgccaaa

SEQ ID NO: 714 - RNA, 20 nt, synthetic construct
  source 1..20: mol_type = other RNA
ggttgtccgagcagtagccc

SEQ ID NO: 715 - DNA, 20 bp, synthetic construct
  source 1..20: mol_type = other DNA
ggttgtccgagcagtagccc

SEQ ID NO: 716 - RNA, 91 nt, synthetic construct
  source 1..91: mol_type = other RNA
ggttgtccgagcagtagcccgttttagagctagaaatagcaagttaaaataaggctagtccgttatcacgaaagggcaccgagtcggtgct

(19) United States
(12) Patent Application Publication
(10) Pub. No.: US 2025/0388890 A1
(43) Pub. Date: Dec. 25, 2025

(54) ASS1 Gene Insertion For The Treatment Of Citrullinemia Type I

(71) Applicant: Regeneron Pharmaceuticals, Inc., Tarrytown, NY (US)

(72) Inventors: Evangelos Pefanis, Somers, NY (US); Leah Sabin, Goldens Bridge, NY (US); Viktoria Gusarova, Pleasantville, NY (US); Eriks Smagris, Yorktown Heights, NY (US); James Foti, Cambridge, MA (US); Arti Mahendra Prakash Kanjolia, Malden, MA (US); Carsten Russ, Cambridge, MA (US)

(21) Appl. No.: 19/244,442

(22) Filed: Jun. 20, 2025

Related U.S. Application Data
(60) Provisional application No. 63/662,169, filed on Jun. 20, 2024.
(60) Provisional application No. 63/710,120, filed on Oct. 22, 2024.

(51) Int. Cl. C12N 9/00; A61K 31/7105; A61K 38/46; A61K 38/53; A61K 48/00; A61P 13/00; C12N 9/22; C12N 15/11; C12N 15/86; C12N 15/88; C12N 15/90

(52) U.S. Cl. CPC C12N 9/93; A61K 31/7105; A61K 38/465; A61K 38/53; A61K 48/005; A61P 13/00; C12N 9/226; C12N 15/11; C12N 15/86; C12N 15/88; C12N 15/907; C12Y 603/04005; C12N 2310/20; C12N 2310/315; C12N 2310/321; C12N 2750/14143

(57) ABSTRACT

Nucleic acid constructs and compositions that allow insertion of an argininosuccinate synthase 1 (ASS1) coding sequence into a target genomic locus such as an endogenous ASS1 locus and/or expression of the ASS1 coding sequence are provided. Also provided are nuclease agents (e.g., targeting an endogenous ASS1 locus) or nucleic acids encoding nuclease agents to facilitate integration of the nucleic acid constructs into a target genomic locus such as an endogenous ASS1 locus. The nucleic acid constructs and compositions can be used in methods of introducing an ASS1 nucleic acid into a cell, methods of integration of an ASS1 nucleic acid into a target genomic locus, methods of expression of ASS1 in a cell, and in methods of treating citrullinemia type I or ASS1 deficiency in a subject.

### CROSS-REFERENCE TO RELATED APPLICATIONS

This application claims the benefit of U.S. Application No. 63/662,169, filed Jun. 20, 2024, and U.S. Application No. 63/710,120, filed Oct. 22, 2024, each of which is herein incorporated by reference in its entirety for all purposes.

### REFERENCE TO A SEQUENCE LISTING SUBMITTED AS AN XML FILE

The Sequence Listing written in file 633469SEQLIST.xml is 734,134 bytes, was created on Jun. 18, 2025, and is hereby incorporated by reference in its entirety.

### BACKGROUND

Citrullinemia type I (CTLN1) is a life-threatening urea cycle disorder impacting approximately 1 in 57,000 live births. It is a monogenic condition arising from deficiency of argininosuccinate synthetase (ASS1). When left untreated, CTLN1 can lead to serious metabolic decompensation, marked elevation in blood ammonia, and early death. Today, newborn screening in the perinatal period helps to rapidly identify individuals with CTLN1, enabling prompt intervention in the form of dietary protein restriction and nitrogen scavenger therapy. However, lifelong dietary protein restriction poses significant adherence challenges, and frequent administration of nitrogen scavenger medication is difficult to administer to young children. Therefore, many patients receiving standard of care continue to suffer from periodic bouts of hyperammonemia and metabolic decompensation that can progressively lead to irreversible neurological, behavioral, and cognitive sequelae. Liver transplantation is often curative but poses its own set of limitations and complications. The monogenic nature and curative potential of liver transplantation make CTLN1 an ideal indication for gene therapy. However, episome-based hepatic AAV gene therapies are often ineffective when administered early in life due to vector dilution. This poses a significant obstacle to episome-based liver-directed gene therapies for CTLN1 where early intervention is critically important to prevent irreversible neurological manifestations of the disease. Accordingly, better methods to treat CTLN1 are required.

### SUMMARY

Provided are compositions comprising a nuclease agent that targets a nuclease target site in an argininosuccinate synthase 1 (ASS1) gene, compositions comprising a nucleic acid construct comprising a first argininosuccinate synthase protein coding sequence, combinations comprising a composition comprising the nuclease agent that targets the nuclease target site in the ASS1 gene and a composition comprising the nucleic acid construct comprising the first argininosuccinate synthase protein coding sequence, cells comprising any of the above compositions or combinations, methods of introducing an argininosuccinate synthase nucleic acid into a cell, methods of integrating an argininosuccinate synthase nucleic acid construct into a target gene in a cell, methods of expressing argininosuccinate synthase in a cell, methods of treating an argininosuccinate synthase deficiency in a subject, methods of treating citrullinemia type I in a subject, and methods of preventing or inhibiting hyperammonemia in a subject having citrullinemia type I.

In one aspect, provided are compositions comprising a nuclease agent that targets a nuclease target site in an argininosuccinate synthase 1 (ASS1) gene. In some such compositions, the ASS1 gene is a human ASS1 gene. In some such compositions, the nuclease target site is in intron 1 or intron 2 of the ASS1 gene. In some such compositions, the nuclease target site is in intron 2 of the ASS1 gene. In some such compositions, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such compositions, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence in an intron of an ASS1 gene, optionally wherein the intron is intron 1 or intron 2, and wherein the guide RNA binds to the Cas protein and targets the Cas protein to the guide RNA target sequence. Similarly, in another aspect, provided are compositions comprising a guide RNA or one or more DNAs encoding the guide RNA, wherein the guide RNA comprises a DNA-targeting segment that targets a guide RNA target sequence in an intron of an ASS1 gene, optionally wherein the intron is intron 1 or intron 2, and wherein the guide RNA binds to a Cas protein and targets the Cas protein to the guide RNA target sequence.

In some such compositions, the ASS1 gene is a human ASS1 gene, and the guide RNA target sequence is in intron 2 of the human ASS1 gene. In some such compositions, the DNA-targeting segment comprises at least 17, at least 18, at least 19, or at least 20 contiguous nucleotides of the sequence set forth in any one of SEQ ID NOS: 31-118, optionally wherein the DNA-targeting segment comprises at least 17, at least 18, at least 19, or at least 20 contiguous nucleotides of the sequence set forth in any one of SEQ ID NOS: 58, 67, 60, 73, 78, 89, 92, and 114, optionally wherein the DNA-targeting segment comprises at least 17, at least 18, at least 19, or at least 20 contiguous nucleotides of the sequence set forth in any one of SEQ ID NOS: 78, 60, and 89, optionally wherein the DNA-targeting segment comprises at least 17, at least 18, at least 19, or at least 20 contiguous nucleotides of the sequence set forth in SEQ ID NO: 78. In some such compositions, the DNA-targeting segment is at least 90% or at least 95% identical to the sequence set forth in any one of SEQ ID NOS: 31-118, optionally wherein the DNA-targeting segment is at least 90% or at least 95% identical to the sequence set forth in any one of SEQ ID NOS: 58, 67, 60, 73, 78, 89, 92, and 114, optionally wherein the DNA-targeting segment is at least 90% or at least 95% identical to the sequence set forth in any one of SEQ ID NOS: 78, 60, and 89, optionally wherein the DNA-targeting segment is at least 90% or at least 95% identical to the sequence set forth in SEQ ID NO: 78. In some such compositions, the DNA-targeting segment comprises any one of SEQ ID NOS: 31-118, optionally wherein the DNA-targeting segment comprises any one of SEQ ID NOS: 58, 67, 60, 73, 78, 89, 92, and 114, optionally wherein the DNA-targeting segment comprises any one of SEQ ID NOS: 78, 60, and 89, optionally wherein the DNA-targeting segment comprises SEQ ID NO: 78. In some such compositions, the DNA-targeting segment consists of any one of SEQ ID NOS: 31-118, optionally wherein the DNA-targeting segment consists of any one of SEQ ID NOS: 58, 67, 60, 73, 78, 89, 92, and 114, optionally wherein the DNA-targeting segment consists of any one of SEQ ID NOS: 78, 60, and 89, optionally wherein the DNA-targeting segment consists of SEQ ID NO: 78. In some such compositions, the guide RNA comprises any one of SEQ ID NOS: 340-427, optionally wherein the guide RNA comprises any one of SEQ ID NOS: 367, 376, 369, 382, 387, 398, 401, and 423, optionally wherein the guide RNA comprises any one of SEQ ID NOS: 387, 369, and 398, optionally wherein the guide RNA comprises SEQ ID NO: 387. In some such compositions, the guide RNA comprises any one of SEQ ID NOS: 252-339, optionally wherein the guide RNA comprises any one of SEQ ID NOS: 279, 288, 281, 294, 299, 310, 313, and 335, optionally wherein the guide RNA comprises any one of SEQ ID NOS: 299, 281, and 310, optionally wherein the guide RNA comprises SEQ ID NO: 299. In some such compositions, the DNA-targeting segment comprises at least 17, at least 18, at least 19, or at least 20 contiguous nucleotides of the sequence set forth in any one of SEQ ID NOS: 31-118, optionally wherein the DNA-targeting segment comprises at least 17, at least 18, at least 19, or at least 20 contiguous nucleotides of the sequence set forth in any one of SEQ ID NOS: 58, 67, 60, 73, 78, 89, 92, and 114, optionally wherein the DNA-targeting segment comprises at least 17, at least 18, at least 19, or at least 20 contiguous nucleotides of the sequence set forth in any one of SEQ ID NOS: 58 and 67. In some such compositions, the DNA-targeting segment is at least 90% or at least 95% identical to the sequence set forth in any one of SEQ ID NOS: 31-118, optionally wherein the DNA-targeting segment is at least 90% or at least 95% identical to the sequence set forth in any one of SEQ ID NOS: 58, 67, 60, 73, 78, 89, 92, and 114, optionally wherein the DNA-targeting segment is at least 90% or at least 95% identical to the sequence set forth in any one of SEQ ID NOS: 58 and 67. In some such compositions, the DNA-targeting segment comprises any one of SEQ ID NOS: 31-118, optionally wherein the DNA-targeting segment comprises any one of SEQ ID NOS: 58, 67, 60, 73, 78, 89, 92, and 114, optionally wherein the DNA-targeting segment comprises any one of SEQ ID NOS: 58 and 67. In some such compositions, the DNA-targeting segment consists of any one of SEQ ID NOS: 31-118, optionally wherein the DNA-targeting segment consists of any one of SEQ ID NOS: 58, 67, 60, 73, 78, 89, 92, and 114, optionally wherein the DNA-targeting segment consists of any one of SEQ ID NOS: 58 and 67. In some such compositions, the guide RNA comprises any one of SEQ ID NOS: 340-427, optionally wherein the guide RNA comprises any one of SEQ ID NOS: 367, 376, 369, 382, 387, 398, 401, and 423, optionally wherein the guide RNA comprises any one of SEQ ID NOS: 367 and 376. In some such compositions, the guide RNA comprises any one of SEQ ID NOS: 252-339, optionally wherein the guide RNA comprises any one of SEQ ID NOS: 279, 288, 281, 294, 299, 310, 313, and 335, optionally wherein the guide RNA comprises any one of SEQ ID NOS: 279 and 288.

In some such compositions, the DNA-targeting segment comprises at least 17, at least 18, at least 19, or at least 20 contiguous nucleotides of SEQ ID NO: 78. In some such compositions, the DNA-targeting segment is at least 90% or at least 95% identical to SEQ ID NO: 78. In some such compositions, the DNA-targeting segment comprises SEQ ID NO: 78. In some such compositions, the DNA-targeting segment consists of SEQ ID NO: 78. In some such compositions, the guide RNA comprises SEQ ID NO: 387. In some such compositions, the guide RNA comprises SEQ ID NO: 299. In some such compositions, the DNA-targeting segment comprises at least 17, at least 18, at least 19, or at least 20 contiguous nucleotides of SEQ ID NO: 58. In some such compositions, the DNA-targeting segment is at least 90% or at least 95% identical to SEQ ID NO: 58. In some such compositions, the DNA-targeting segment comprises SEQ ID NO: 58. In some such compositions, the DNA-targeting segment consists of SEQ ID NO: 58. In some such compositions, the guide RNA comprises SEQ ID NO: 367. In some such compositions, the guide RNA comprises SEQ ID NO: 279.

In some such compositions, the ASS1 gene is a human ASS1 gene, and the guide RNA target sequence is in intron 1 of the human ASS1 gene. In some such compositions, the DNA-targeting segment comprises at least 17, at least 18, at least 19, or at least 20 contiguous nucleotides of the sequence set forth in any one of SEQ ID NOS: 430-517. In some such compositions, the DNA-targeting segment is at least 90% or at least 95% identical to the sequence set forth in any one of SEQ ID NOS: 430-517. In some such compositions, the DNA-targeting segment comprises any one of SEQ ID NOS: 430-517. In some such compositions, the DNA-targeting segment consists of any one of SEQ ID NOS: 430-517. In some such compositions, the guide RNA comprises any one of SEQ ID NOS: 606-693.

In some such compositions, the composition comprises the guide RNA in the form of RNA. In some such compositions, the guide RNA comprises at least one modification. In some such compositions, the at least one modification comprises a 2′-O-methyl-modified nucleotide. In some such compositions, the at least one modification comprises a phosphorothioate bond between nucleotides. In some such compositions, the at least one modification comprises a modification at one or more of the first five nucleotides at the 5′ end of the guide RNA. In some such compositions, the at least one modification comprises a modification at one or more of the last five nucleotides at the 3′ end of the guide RNA. In some such compositions, the at least one modification comprises phosphorothioate bonds between the first four nucleotides at the 5′ end of the guide RNA. In some such compositions, the at least one modification comprises phosphorothioate bonds between the last four nucleotides at the 3′ end of the guide RNA. In some such compositions, the at least one modification comprises 2′-O-methyl-modified nucleotides at the first three nucleotides at the 5′ end of the guide RNA. In some such compositions, the at least one modification comprises 2′-O-methyl-modified nucleotides at the last three nucleotides at the 3′ end of the guide RNA. In some such compositions, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such compositions, the guide RNA is a single guide RNA (sgRNA). In some such compositions, the guide RNA has the modification pattern set forth in SEQ ID NO: 429. In some such compositions, the guide RNA has the modification pattern set forth in SEQ ID NO: 30.

In some such compositions, the composition comprises the guide RNA in the form of RNA, the guide RNA comprises SEQ ID NO: 387, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such compositions, the composition comprises the guide RNA in the form of RNA, the guide RNA comprises SEQ ID NO: 387, and the guide RNA has the modification pattern set forth in SEQ ID NO: 429. In some such compositions, the composition comprises the guide RNA in the form of RNA, the guide RNA comprises SEQ ID NO: 299, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such compositions, the composition comprises the guide RNA in the form of RNA, the guide RNA comprises SEQ ID NO: 299, and the guide RNA has the modification pattern set forth in SEQ ID NO: 30. In some such compositions, the composition comprises the guide RNA in the form of RNA, the guide RNA comprises SEQ ID NO: 367, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such compositions, the composition comprises the guide RNA in the form of RNA, the guide RNA comprises SEQ ID NO: 367, and the guide RNA has the modification pattern set forth in SEQ ID NO: 429. In some such compositions, the composition comprises the guide RNA in the form of RNA, the guide RNA comprises SEQ ID NO: 279, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such compositions, the composition comprises the guide RNA in the form of RNA, the guide RNA comprises SEQ ID NO: 279, and the guide RNA has the modification pattern set forth in SEQ ID NO: 30.

In some such compositions, the Cas protein is a Cas9 protein. In some such compositions, the Cas protein is, or is derived from, a Streptococcus pyogenes Cas9 protein. In some such compositions, the Cas protein comprises the sequence set forth in SEQ ID NO: 12.

In some such composition, the composition further comprises the Cas protein or a nucleic acid encoding the Cas protein. In some such compositions, the nucleic acid encoding the Cas protein is codon-optimized for expression in a mammalian cell or a human cell. In some such compositions, the composition comprises the nucleic acid encoding the Cas protein, wherein the nucleic acid comprises an mRNA encoding the Cas protein. In some such compositions, the mRNA encoding the Cas protein comprises at least one modification. In some such compositions, the mRNA encoding the Cas protein is modified to comprise a modified uridine at one or more or all uridine positions. In some such compositions, the modified uridine is N1-methyl-pseudouridine. In some such compositions, the mRNA encoding the Cas protein is fully substituted with N1-methyl-pseudouridine. In some such compositions, the mRNA encoding the Cas protein comprises a 5′ cap. In some such compositions, the mRNA encoding the Cas protein comprises a poly(A) tail. In some such compositions, the mRNA encoding the Cas protein comprises the sequence set forth in SEQ ID NO: 250 or 249. In some such compositions, the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail.

In some such compositions, the composition comprises the guide RNA in the form of RNA, and the guide RNA comprises SEQ ID NO: 387, and the composition comprises the nucleic acid encoding the Cas protein, wherein the nucleic acid comprises an mRNA encoding the Cas protein, and the mRNA encoding the Cas protein comprises the sequence set forth in SEQ ID NO: 250 or 249. In some such compositions, the composition comprises the guide RNA in the form of RNA, the guide RNA comprises SEQ ID NO: 387, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail. In some such compositions, the composition comprises the guide RNA in the form of RNA, the guide RNA comprises SEQ ID NO: 387, and the guide RNA has the modification pattern set forth in SEQ ID NO: 429, and the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail. In some such compositions, the composition comprises the guide RNA in the form of RNA, and the guide RNA comprises SEQ ID NO: 299, and the composition comprises the nucleic acid encoding the Cas protein, wherein the nucleic acid comprises an mRNA encoding the Cas protein, and the mRNA encoding the Cas protein comprises the sequence set forth in SEQ ID NO: 250 or 249. In some such compositions, the composition comprises the guide RNA in the form of RNA, the guide RNA comprises SEQ ID NO: 299, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail. In some such compositions, the composition comprises the guide RNA in the form of RNA, the guide RNA comprises SEQ ID NO: 299, and the guide RNA has the modification pattern set forth in SEQ ID NO: 30, and the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail. In some such compositions, the composition comprises the guide RNA in the form of RNA, and the guide RNA comprises SEQ ID NO: 367, and the composition comprises the nucleic acid encoding the Cas protein, wherein the nucleic acid comprises an mRNA encoding the Cas protein, and the mRNA encoding the Cas protein comprises the sequence set forth in SEQ ID NO: 250 or 249. In some such compositions, the composition comprises the guide RNA in the form of RNA, the guide RNA comprises SEQ ID NO: 367, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail. In some such compositions, the composition comprises the guide RNA in the form of RNA, the guide RNA comprises SEQ ID NO: 367, and the guide RNA has the modification pattern set forth in SEQ ID NO: 429, and the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail. In some such compositions, the composition comprises the guide RNA in the form of RNA, and the guide RNA comprises SEQ ID NO: 279, and the composition comprises the nucleic acid encoding the Cas protein, wherein the nucleic acid comprises an mRNA encoding the Cas protein, and the mRNA encoding the Cas protein comprises the sequence set forth in SEQ ID NO: 250 or 249. In some such compositions, the composition comprises the guide RNA in the form of RNA, the guide RNA comprises SEQ ID NO: 279, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail. In some such compositions, the composition comprises the guide RNA in the form of RNA, the guide RNA comprises SEQ ID NO: 279, and the guide RNA has the modification pattern set forth in SEQ ID NO: 30, and the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail.

In some such compositions, the Cas protein or the nucleic acid encoding the Cas protein and the guide RNA or the one or more DNAs encoding the guide RNA are associated with a lipid nanoparticle. In some such compositions, the lipid nanoparticle comprises a cationic lipid, a neutral lipid, a helper lipid, and a stealth lipid. In some such compositions, the cationic lipid is Lipid A. In some such compositions, the neutral lipid is DSPC. In some such compositions, the helper lipid is cholesterol. In some such compositions, the stealth lipid is 1,2-dimyristoyl-rac-glycero-3-methoxypolyethylene glycol-2000 (PEG2k-DMG). In some such compositions, the cationic lipid is Lipid A, the neutral lipid is DSPC, the helper lipid is cholesterol, and the stealth lipid is PEG2k-DMG. In some such compositions, the lipid nanoparticle comprises four lipids at the following molar ratios: about 50 mol % Lipid A, about 9 mol % DSPC, about 38 mol % cholesterol, and about 3 mol % PEG2k-DMG.

In some such compositions, the ASS1 gene is a human ASS1 gene, the composition comprises the guide RNA in the form of RNA, and the guide RNA comprises SEQ ID NO: 387, the composition comprises the nucleic acid encoding the Cas protein, wherein the nucleic acid comprises an mRNA encoding the Cas protein, and the mRNA encoding the Cas protein comprises the sequence set forth in SEQ ID NO: 250 or 249, and the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the ASS1 gene is a human ASS1 gene, the composition comprises the guide RNA in the form of RNA, the guide RNA comprises SEQ ID NO: 387, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail, and the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the ASS1 gene is a human ASS1 gene, the composition comprises the guide RNA in the form of RNA, the guide RNA comprises SEQ ID NO: 387, and the guide RNA has the modification pattern set forth in SEQ ID NO: 429, the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail, and the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the ASS1 gene is a human ASS1 gene, the composition comprises the guide RNA in the form of RNA, and the guide RNA comprises SEQ ID NO: 367, the composition comprises the nucleic acid encoding the Cas protein, wherein the nucleic acid comprises an mRNA encoding the Cas protein, and the mRNA encoding the Cas protein comprises the sequence set forth in SEQ ID NO: 250 or 249, and the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the ASS1 gene is a human ASS1 gene, the composition comprises the guide RNA in the form of RNA, the guide RNA comprises SEQ ID NO: 367, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail, and the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the ASS1 gene is a human ASS1 gene, the composition comprises the guide RNA in the form of RNA, the guide RNA comprises SEQ ID NO: 367, and the guide RNA has the modification pattern set forth in SEQ ID NO: 429, the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail, and the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG.

In some such compositions, the ASS1 gene is a human ASS1 gene, the composition comprises the guide RNA in the form of RNA, and the guide RNA comprises SEQ ID NO: 299, the composition comprises the nucleic acid encoding the Cas protein, wherein the nucleic acid comprises an mRNA encoding the Cas protein, and the mRNA encoding the Cas protein comprises the sequence set forth in SEQ ID NO: 250 or 249, and the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the ASS1 gene is a human ASS1 gene, the composition comprises the guide RNA in the form of RNA, the guide RNA comprises SEQ ID NO: 299, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail, and the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the ASS1 gene is a human ASS1 gene, the composition comprises the guide RNA in the form of RNA, the guide RNA comprises SEQ ID NO: 299, and the guide RNA has the modification pattern set forth in SEQ ID NO: 30, the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail, and the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the ASS1 gene is a human ASS1 gene, the composition comprises the guide RNA in the form of RNA, and the guide RNA comprises SEQ ID NO: 279, the composition comprises the nucleic acid encoding the Cas protein, wherein the nucleic acid comprises an mRNA encoding the Cas protein, and the mRNA encoding the Cas protein comprises the sequence set forth in SEQ ID NO: 250 or 249, and the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the ASS1 gene is a human ASS1 gene, the composition comprises the guide RNA in the form of RNA, the guide RNA comprises SEQ ID NO: 279, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail, and the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the ASS1 gene is a human ASS1 gene, the composition comprises the guide RNA in the form of RNA, the guide RNA comprises SEQ ID NO: 279, and the guide RNA has the modification pattern set forth in SEQ ID NO: 30, the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail, and the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG.

In another aspect, provided are compositions comprising a nucleic acid construct comprising a first argininosuccinate synthase protein coding sequence. In some such compositions, the first argininosuccinate synthase protein coding sequence is a human argininosuccinate synthase protein coding sequence. In some such compositions, the first argininosuccinate synthase protein coding sequence comprises human argininosuccinate synthase exons 2-14 or exons 3-14. In some such compositions, the first argininosuccinate synthase protein coding sequence comprises human argininosuccinate synthase exons 3-14. In some such compositions, the first argininosuccinate synthase protein coding sequence encodes a protein at last 95%, at least 96%, at least 97%, at least 98%, or at least 99% identical to the sequence set forth in SEQ ID NO: 230. In some such compositions, the first argininosuccinate synthase protein coding sequence encodes a protein comprising the sequence set forth in SEQ ID NO: 230. In some such compositions, the first argininosuccinate synthase protein coding sequence encodes a protein consisting of the sequence set forth in SEQ ID NO: 230. In some such compositions, the first argininosuccinate synthase protein coding sequence is at least 96%, 97%, 98%, or 99% identical to any one of SEQ ID NOS: 705, 709, and 713. In some such compositions, the first argininosuccinate synthase protein coding sequence is at least 90%, 95%, 96%, 97%, 98%, or 99% identical to any one of SEQ ID NOS: 706 and 710. In some such compositions, the first argininosuccinate synthase protein coding sequence is at least 99% identical to any one of SEQ ID NOS: 705, 706, 709, 710, and 713. In some such compositions, the first argininosuccinate synthase protein coding sequence comprises, consists essentially of, or consists of any one of SEQ ID NOS: 705, 706, 709, 710, and 713. In some such compositions, the first argininosuccinate synthase protein coding sequence is at least 96%, 97%, 98%, or 99% identical to SEQ ID NO: 705. In some such compositions, the first argininosuccinate synthase protein coding sequence is at least 99% identical to SEQ ID NO: 705. In some such compositions, the first argininosuccinate synthase protein coding sequence comprises, consists essentially of, or consists of SEQ ID NO: 705. In some such compositions, the first argininosuccinate synthase protein coding sequence is at least 90%, 95%, 96%, 97%, 98%, or 99% identical to SEQ ID NO: 706. In some such compositions, the first argininosuccinate synthase protein coding sequence is at least 99% identical to SEQ ID NO: 706. In some such compositions, the first argininosuccinate synthase protein coding sequence comprises, consists essentially of, or consists of SEQ ID NO: 706.

In some such compositions, the first argininosuccinate synthase protein coding sequence comprises human argininosuccinate synthase exons 2-14. In some such compositions, the first argininosuccinate synthase protein coding sequence encodes a protein at last 95%, at least 96%, at least 97%, at least 98%, or at least 99% identical to the sequence set forth in SEQ ID NO: 227. In some such compositions, the first argininosuccinate synthase protein coding sequence encodes a protein comprising the sequence set forth in SEQ ID NO: 227. In some such compositions, the first argininosuccinate synthase protein coding sequence encodes a protein consisting of the sequence set forth in SEQ ID NO: 227.

In some such compositions, the nucleic acid construct comprises a splice acceptor upstream of the first argininosuccinate synthase protein coding sequence. In some such compositions, the nucleic acid construct comprises a polyadenylation signal downstream of the first argininosuccinate synthase protein coding sequence. In some such compositions, the nucleic acid construct comprises a splice acceptor upstream of the first argininosuccinate synthase protein coding sequence, and the nucleic acid construct comprises a polyadenylation signal downstream of the first argininosuccinate synthase protein coding sequence. In some such compositions, the nucleic acid construct does not comprise homology arms. In some such compositions, the nucleic acid construct comprises homology arms. In some such compositions, the nucleic acid construct does not comprise a promoter that drives the expression of the argininosuccinate synthase protein.

In some such compositions, the nucleic acid construct is a bidirectional construct. In some such compositions, the nucleic acid construct comprises the first argininosuccinate synthase protein coding sequence and a reverse complement of a second argininosuccinate synthase protein coding sequence. In some such compositions, the first argininosuccinate synthase protein coding sequence and the second argininosuccinate synthase protein coding sequence are different but encode the same argininosuccinate synthase protein sequence. In some such compositions, the first argininosuccinate synthase protein coding sequence and the second argininosuccinate synthase protein coding sequence each encode a protein at least 95%, at least 96%, at least 97%, at least 98%, or at least 99% identical to the sequence set forth in SEQ ID NO: 230. In some such compositions, the first argininosuccinate synthase protein coding sequence and the second argininosuccinate synthase protein coding sequence each encode a protein comprising the sequence set forth in SEQ ID NO: 230. In some such compositions, the first argininosuccinate synthase protein coding sequence and the second argininosuccinate synthase protein coding sequence each encode a protein consisting of the sequence set forth in SEQ ID NO: 230. In some such compositions, the first argininosuccinate synthase protein coding sequence is at least 96%, 97%, 98%, or 99% identical to any one of SEQ ID NOS: 705, 709, and 713. Optionally, the first argininosuccinate synthase protein coding sequence is at least 96%, 97%, 98%, or 99% identical to SEQ ID NO: 705. In some such compositions, the first argininosuccinate synthase protein coding sequence is at least 99% identical to any one of SEQ ID NOS: 705, 709, and 713. Optionally, the first argininosuccinate synthase protein coding sequence is at least 99% identical to SEQ ID NO: 705. In some such compositions, the first argininosuccinate synthase protein coding sequence comprises, consists essentially of, or consists of any one of SEQ ID NOS: 705, 709, and 713. Optionally, the first argininosuccinate synthase protein coding sequence comprises, consists essentially of, or consists of SEQ ID NO: 705. In some such compositions, the second argininosuccinate synthase protein coding sequence is at least 90%, 95%, 96%, 97%, 98%, or 99% identical to any one of SEQ ID NOS: 706 and 710. Optionally, the second argininosuccinate synthase protein coding sequence is at least 90%, 95%, 96%, 97%, 98%, or 99% identical to SEQ ID NO: 706. In some such compositions, the second argininosuccinate synthase protein coding sequence is at least 99% identical to any one of SEQ ID NOS: 706 and 710. Optionally, the second argininosuccinate synthase protein coding sequence is at least 99% identical to SEQ ID NO: 706. In some such compositions, the second argininosuccinate synthase protein coding sequence comprises, consists essentially of, or consists of any one of SEQ ID NOS: 706 and 710. Optionally, the second argininosuccinate synthase protein coding sequence comprises, consists essentially of, or consists of SEQ ID NO: 706. In some such compositions, the first argininosuccinate synthase protein coding sequence is at least 96%, 97%, 98%, or 99% identical to SEQ ID NO: 705, and the second argininosuccinate synthase protein coding sequence is at least 90%, 95%, 96%, 97%, 98%, or 99% identical to SEQ ID NO: 706. In some such compositions, the first argininosuccinate synthase protein coding sequence is at least 99% identical to SEQ ID NO: 705, and the second argininosuccinate synthase protein coding sequence is at least 99% identical to SEQ ID NO: 706. In some such compositions, the first argininosuccinate synthase protein coding sequence comprises, consists essentially of, or consists of SEQ ID NO: 705, and the second argininosuccinate synthase protein coding sequence comprises, consists essentially of, or consists of SEQ ID NO: 706. In some such compositions, the first argininosuccinate synthase protein coding sequence is at least 96%, 97%, 98%, or 99% identical to SEQ ID NO: 709, and the second argininosuccinate synthase protein coding sequence is at least 90%, 95%, 96%, 97%, 98%, or 99% identical to SEQ ID NO: 710. In some such compositions, the first argininosuccinate synthase protein coding sequence is at least 99% identical to SEQ ID NO: 709, and the second argininosuccinate synthase protein coding sequence is at least 99% identical to SEQ ID NO: 710. In some such compositions, the first argininosuccinate synthase protein coding sequence comprises, consists essentially of, or consists of SEQ ID NO: 709, and the second argininosuccinate synthase protein coding sequence comprises, consists essentially of, or consists of SEQ ID NO: 710. In some such compositions, the first argininosuccinate synthase protein coding sequence is at least 96%, 97%, 98%, or 99% identical to SEQ ID NO: 713, and the second argininosuccinate synthase protein coding sequence is at least 90%, 95%, 96%, 97%, 98%, or 99% identical to SEQ ID NO: 706. In some such compositions, the first argininosuccinate synthase protein coding sequence is at least 99% identical to SEQ ID NO: 713, and the second argininosuccinate synthase protein coding sequence is at least 99% identical to SEQ ID NO: 706. In some such compositions, the first argininosuccinate synthase protein coding sequence comprises, consists essentially of, or consists of SEQ ID NO: 713, and the second argininosuccinate synthase protein coding sequence comprises, consists essentially of, or consists of SEQ ID NO: 706.

In some such compositions, the first argininosuccinate synthase protein coding sequence and the second argininosuccinate synthase protein coding sequence each encode a protein at least 95%, at least 96%, at least 97%, at least 98%, or at least 99% identical to the sequence set forth in SEQ ID NO: 227. In some such compositions, the first argininosuccinate synthase protein coding sequence and the second argininosuccinate synthase protein coding sequence each encode a protein comprising the sequence set forth in SEQ ID NO: 227. In some such compositions, the first argininosuccinate synthase protein coding sequence and the second argininosuccinate synthase protein coding sequence each encode a protein consisting of the sequence set forth in SEQ ID NO: 227. In some such compositions, the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor, or wherein the nucleic acid construct comprises from 5′ to 3′: a first splice acceptor, the second argininosuccinate synthase protein coding sequence, a first polyadenylation signal, a reverse complement of a second polyadenylation signal, the reverse complement of the first argininosuccinate synthase protein coding sequence, and a reverse complement of a second splice acceptor. In some such compositions, the first argininosuccinate synthase protein coding sequence and the second argininosuccinate synthase protein coding sequence are different but encode the same argininosuccinate synthase protein sequence, and wherein the first polyadenylation signal and the second polyadenylation signal are different.

In some such compositions, the nucleic acid construct is a bidirectional construct comprising the first argininosuccinate synthase protein coding sequence and a reverse complement of a second argininosuccinate synthase protein coding sequence, the first argininosuccinate synthase protein coding sequence and the second argininosuccinate synthase protein coding sequence are different and each encode a protein comprising or consisting of the sequence set forth in SEQ ID NO: 230, the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor, the nucleic acid construct does not comprise a promoter that drives the expression of the argininosuccinate synthase protein, and the nucleic acid construct does not comprise homology arms. In some such compositions, the nucleic acid construct is a bidirectional construct comprising the first argininosuccinate synthase protein coding sequence and a reverse complement of a second argininosuccinate synthase protein coding sequence, the first argininosuccinate synthase protein coding sequence and the second argininosuccinate synthase protein coding sequence are different and each encode a protein comprising or consisting of the sequence set forth in SEQ ID NO: 230, the first argininosuccinate synthase protein coding sequence comprises SEQ ID NO: 705, the second argininosuccinate synthase protein coding sequence comprises SEQ ID NO: 706, the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor, the nucleic acid construct does not comprise a promoter that drives the expression of the argininosuccinate synthase protein, and the nucleic acid construct does not comprise homology arms. In some such compositions, the nucleic acid construct is a bidirectional construct comprising the first argininosuccinate synthase protein coding sequence and a reverse complement of a second argininosuccinate synthase protein coding sequence, the first argininosuccinate synthase protein coding sequence and the second argininosuccinate synthase protein coding sequence are different and each encode a protein comprising or consisting of the sequence set forth in SEQ ID NO: 230, the first argininosuccinate synthase protein coding sequence comprises SEQ ID NO: 709, the second argininosuccinate synthase protein coding sequence comprises SEQ ID NO: 710, the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor, the nucleic acid construct does not comprise a promoter that drives the expression of the argininosuccinate synthase protein, and the nucleic acid construct does not comprise homology arms. In some such compositions, the nucleic acid construct is a bidirectional construct comprising the first argininosuccinate synthase protein coding sequence and a reverse complement of a second argininosuccinate synthase protein coding sequence, the first argininosuccinate synthase protein coding sequence and the second argininosuccinate synthase protein coding sequence are different and each encode a protein comprising or consisting of the sequence set forth in SEQ ID NO: 230, the first argininosuccinate synthase protein coding sequence comprises SEQ ID NO: 713, the second argininosuccinate synthase protein coding sequence comprises SEQ ID NO: 706, the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor, the nucleic acid construct does not comprise a promoter that drives the expression of the argininosuccinate synthase protein, and the nucleic acid construct does not comprise homology arms. In some such compositions, the nucleic acid construct is a bidirectional construct comprising the first argininosuccinate synthase protein coding sequence and a reverse complement of a second argininosuccinate synthase protein coding sequence, the first argininosuccinate synthase protein coding sequence and the second argininosuccinate synthase protein coding sequence are different and each encode a protein comprising or consisting of the sequence set forth in SEQ ID NO: 227, the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor, the nucleic acid construct does not comprise a promoter that drives the expression of the argininosuccinate synthase protein, and the nucleic acid construct does not comprise homology arms.

In some such compositions, the nucleic acid construct is a unidirectional construct. In some such compositions, the nucleic acid construct is a unidirectional construct comprising the first argininosuccinate synthase protein coding sequence, the first argininosuccinate synthase protein coding sequence encodes a protein comprising or consisting of the sequence set forth in SEQ ID NO: 230, the nucleic acid construct comprises from 5′ to 3′: a splice acceptor, the first argininosuccinate synthase protein coding sequence, and a polyadenylation signal, the nucleic acid construct does not comprise a promoter that drives the expression of the argininosuccinate synthase protein, and the nucleic acid construct does not comprise homology arms. In some such compositions, the nucleic acid construct is a unidirectional construct comprising the first argininosuccinate synthase protein coding sequence, the first argininosuccinate synthase protein coding sequence encodes a protein comprising or consisting of the sequence set forth in SEQ ID NO: 227, the nucleic acid construct comprises from 5′ to 3′: a splice acceptor, the first argininosuccinate synthase protein coding sequence, and a polyadenylation signal, the nucleic acid construct does not comprise a promoter that drives the expression of the argininosuccinate synthase protein, and the nucleic acid construct does not comprise homology arms.

In some such compositions, the nucleic acid construct is single-stranded DNA or double-stranded DNA. In some such compositions, the nucleic acid construct is single-stranded DNA. In some such compositions, the nucleic acid construct is in a nucleic acid vector or a lipid nanoparticle. In some such compositions, the nucleic acid construct is in the nucleic acid vector, optionally wherein the nucleic acid vector is a viral vector. In some such compositions, the nucleic acid vector is an adeno-associated viral (AAV) vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the AAV vector is a single-stranded AAV (ssAAV) vector. In some such compositions, the AAV vector is derived from an AAV8 vector, an AAV3B vector, an AAV5 vector, an AAV6 vector, an AAV7 vector, an AAV9 vector, an AAVrh.74 vector, or an AAVhu.37 vector. In some such compositions, the AAV vector is a recombinant AAV8 (rAAV8) vector. In some such compositions, the AAV vector is a single-stranded rAAV8 vector. In some such compositions, the AAV vector is a recombinant AAV5 (rAAV5) vector. In some such compositions, the AAV vector is a single-stranded rAAV5 vector.

In some such compositions, the nucleic acid construct is a bidirectional construct comprising the first argininosuccinate synthase protein coding sequence and a reverse complement of a second argininosuccinate synthase protein coding sequence, the first argininosuccinate synthase protein coding sequence and the second argininosuccinate synthase protein coding sequence are different and each encode a protein comprising or consisting of the sequence set forth in SEQ ID NO: 230, the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor, the nucleic acid construct does not comprise a promoter that drives the expression of the argininosuccinate synthase protein, the nucleic acid construct does not comprise homology arms, and the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a bidirectional construct comprising the first argininosuccinate synthase protein coding sequence and a reverse complement of a second argininosuccinate synthase protein coding sequence, the first argininosuccinate synthase protein coding sequence and the second argininosuccinate synthase protein coding sequence are different and each encode a protein comprising or consisting of the sequence set forth in SEQ ID NO: 230, the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor, the nucleic acid construct does not comprise a promoter that drives the expression of the argininosuccinate synthase protein, the nucleic acid construct does not comprise homology arms, and the nucleic acid construct is in a single-stranded rAAV5 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a bidirectional construct comprising the first argininosuccinate synthase protein coding sequence and a reverse complement of a second argininosuccinate synthase protein coding sequence, the first argininosuccinate synthase protein coding sequence and the second argininosuccinate synthase protein coding sequence are different and each encode a protein comprising or consisting of the sequence set forth in SEQ ID NO: 230, the first argininosuccinate synthase protein coding sequence comprises SEQ ID NO: 705, the second argininosuccinate synthase protein coding sequence comprises SEQ ID NO: 706, the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor, the nucleic acid construct does not comprise a promoter that drives the expression of the argininosuccinate synthase protein, the nucleic acid construct does not comprise homology arms, and the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a bidirectional construct comprising the first argininosuccinate synthase protein coding sequence and a reverse complement of a second argininosuccinate synthase protein coding sequence, the first argininosuccinate synthase protein coding sequence and the second argininosuccinate synthase protein coding sequence are different and each encode a protein comprising or consisting of the sequence set forth in SEQ ID NO: 230, the first argininosuccinate synthase protein coding sequence comprises SEQ ID NO: 709, the second argininosuccinate synthase protein coding sequence comprises SEQ ID NO: 710, the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor, the nucleic acid construct does not comprise a promoter that drives the expression of the argininosuccinate synthase protein, the nucleic acid construct does not comprise homology arms, and the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a bidirectional construct comprising the first argininosuccinate synthase protein coding sequence and a reverse complement of a second argininosuccinate synthase protein coding sequence, the first argininosuccinate synthase protein coding sequence and the second argininosuccinate synthase protein coding sequence are different and each encode a protein comprising or consisting of the sequence set forth in SEQ ID NO: 230, the first argininosuccinate synthase protein coding sequence comprises SEQ ID NO: 713, the second argininosuccinate synthase protein coding sequence comprises SEQ ID NO: 706, the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor, the nucleic acid construct does not comprise a promoter that drives the expression of the argininosuccinate synthase protein, the nucleic acid construct does not comprise homology arms, and the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a bidirectional construct comprising the first argininosuccinate synthase protein coding sequence and a reverse complement of a second argininosuccinate synthase protein coding sequence, the first argininosuccinate synthase protein coding sequence and the second argininosuccinate synthase protein coding sequence are different and each encode a protein comprising or consisting of the sequence set forth in SEQ ID NO: 227, the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor, the nucleic acid construct does not comprise a promoter that drives the expression of the argininosuccinate synthase protein, the nucleic acid construct does not comprise homology arms, and the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196.

In another aspect is a combination comprising: (I) any of the above compositions comprising the nuclease agent that targets the nuclease target site in the ASS1 gene; and (II) any of the above compositions comprising the nucleic acid construct comprising the first argininosuccinate synthase protein coding sequence.

In another aspect, provided are any of the above compositions or any of the above combinations for use in a method of introducing an argininosuccinate synthase (ASS1) nucleic acid into a cell, a method of integrating an ASS1 nucleic acid construct into a target gene in a cell, or a method of expressing argininosuccinate synthase in a cell. In some such compositions or combinations for use, the cell is a neonatal cell. In some such compositions or combinations for use, the neonatal cell is from a human neonatal subject within 24 weeks after birth, is from a human neonatal subject within 12 weeks after birth, is from a human neonatal subject within 8 weeks after birth, is from a human neonatal subject within 4 weeks after birth, is from a human neonatal subject within 2 weeks after birth, or is from a human neonatal subject within 1 week after birth. In some such compositions or combinations for use, the cell is not a neonatal cell.

In another aspect, provided are uses any of the above compositions or any of the above combinations in the preparation of a reagent for introducing an argininosuccinate synthase (ASS1) nucleic acid into a cell, integrating an ASS1 nucleic acid construct into a target gene in a cell, or expressing argininosuccinate synthase in a cell. In some such uses, the cell is a neonatal cell. In some such uses, the neonatal cell is from a human neonatal subject within 24 weeks after birth, is from a human neonatal subject within 12 weeks after birth, is from a human neonatal subject within 8 weeks after birth, is from a human neonatal subject within 4 weeks after birth, is from a human neonatal subject within 2 weeks after birth, or is from a human neonatal subject within 1 week after birth. In some such uses, the cell is not a neonatal cell.

In another aspect, provided are any of the above compositions or any of the above combinations for use in a method of treating an argininosuccinate synthase deficiency in a subject. In another aspect, provided are any of the above compositions or any of the above combinations for use in a method of treating citrullinemia type I in a subject. In some such compositions or combinations for use, the subject is a neonatal subject. In some such compositions or combinations for use, the neonatal subject is a human neonatal subject within 24 weeks after birth, is a human neonatal subject within 12 weeks after birth, is a human neonatal subject within 8 weeks after birth, is a human neonatal subject within 4 weeks after birth, is a human neonatal subject within 2 weeks after birth, or is a human neonatal subject within 1 week after birth. In some such compositions or combinations for use, the subject is not a neonatal subject.

In another aspect, provided are uses of any of the above compositions or any of the above combinations in the preparation of a medicament treating an argininosuccinate synthase deficiency in a subject. In another aspect, provided are uses of any of the above compositions or any of the above combinations in the preparation of a medicament treating citrullinemia type I in a subject. In some such uses, the subject is a neonatal subject. In some such uses, the neonatal subject is a human neonatal subject within 24 weeks after birth, is a human neonatal subject within 12 weeks after birth, is a human neonatal subject within 8 weeks after birth, is a human neonatal subject within 4 weeks after birth, is a human neonatal subject within 2 weeks after birth, or is a human neonatal subject within 1 week after birth. In some such uses, the subject is not a neonatal subject.

In another aspect, provided are cells comprising any of the above compositions or any of the above combinations. In some such cells, the nucleic acid construct is integrated into an endogenous target gene locus, and wherein argininosuccinate synthase protein is expressed from the endogenous target gene locus, or wherein the nucleic acid construct is integrated into intron 1 or intron 2 of an endogenous argininosuccinate synthase (ASS1) locus, and wherein argininosuccinate synthase protein is expressed from the endogenous ASS1 locus. In some such cells, the cell is a human cell, optionally wherein the nucleic acid construct is integrated into intron 2 of the endogenous ASS1 locus. In some such cells, the cell is a liver cell. In some such cells, the liver cell is a hepatocyte. In some such cells, the cell is a neonatal cell. In some such cells, the neonatal cell is from a human neonatal subject within 24 weeks after birth, is from a human neonatal subject within 12 weeks after birth, is from a human neonatal subject within 8 weeks after birth, is from a human neonatal subject within 4 weeks after birth, is from a human neonatal subject within 2 weeks after birth, or is from a human neonatal subject within 1 week after birth. In some such cells, the cell is not a neonatal cell. In some such cells, the cell is ex vivo or in vitro. In some such cells, the cell is in vivo.

In another aspect, provided are methods of introducing an argininosuccinate synthase nucleic acid into a cell, comprising administering any of the above combinations to the cell. In another aspect, provided are methods of integrating an argininosuccinate synthase nucleic acid construct into a target gene in a cell, comprising administering any of the above combinations to the cell, wherein the nuclease agent cleaves the nuclease target site in the target gene to create a cleavage site, the nucleic acid construct is inserted into the cleavage site to create a modified target gene, and argininosuccinate synthase protein is expressed from the modified target gene. In another aspect, provided are methods of expressing argininosuccinate synthase in a cell, comprising administering any of the above combinations to the cell, wherein the nuclease agent cleaves the nuclease target site in the target gene to create a cleavage site, the nucleic acid construct is inserted into the cleavage site to create a modified target gene, and argininosuccinate synthase protein is expressed from the modified target gene.

In some such methods, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such methods, the nucleic acid construct, the Cas protein or the nucleic acid encoding the Cas protein, and the guide RNA or the one or more DNAs encoding the guide RNAs are administered simultaneously. In some such methods, the nucleic acid construct is not administered simultaneously with the Cas protein or the nucleic acid encoding the Cas protein and the guide RNA or the one or more DNAs encoding the guide RNAs.

In some such methods, the cell is a liver cell. In some such methods, the cell is a hepatocyte. In some such methods, the cell is a human cell. In some such methods, the cell is a neonatal cell. In some such methods, the neonatal cell is from a human neonatal subject within 24 weeks after birth, a human neonatal subject within 12 weeks after birth, a human neonatal subject within 8 weeks after birth, a human neonatal subject within 4 weeks after birth, a human neonatal subject within 2 weeks after birth, or a human neonatal subject within 1 week after birth. In some such methods, the cell is not a neonatal cell. In some such methods, the cell is in vivo. In some such methods, the cell is in vitro or ex vivo.

In another aspect, provided are methods of treating an argininosuccinate synthase deficiency in a subject, comprising administering any of the above combinations to the subject. In another aspect, provided are methods of treating citrullinemia type I in a subject, comprising administering any of the above combinations to the subject. In another aspect, provided are methods of preventing or inhibiting hyperammonemia in a subject having citrullinemia type I, comprising administering any of the above combinations to the subject.

In some such methods, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such methods, the nucleic acid construct, the Cas protein or the nucleic acid encoding the Cas protein, and the guide RNA or the one or more DNAs encoding the guide RNAs are administered simultaneously. In some such methods, the nucleic acid construct is not administered simultaneously with the Cas protein or the nucleic acid encoding the Cas protein and the guide RNA or the one or more DNAs encoding the guide RNAs.

In some such methods, the subject is a neonatal subject. In some such methods, the neonatal subject is a human neonatal subject within 24 weeks after birth, a human neonatal subject is within 12 weeks after birth, a human neonatal subject is within 8 weeks after birth, a human neonatal subject is within 4 weeks after birth, a human neonatal subject is within 2 weeks after birth, or a human neonatal subject is within 1 week after birth. In some such methods, the subject is not a neonatal subject. In some such methods, the subject is a human subject.

In some such methods, the method decreases plasma ammonia and/or plasma citrulline levels in the subject. In some such methods, the method reduces plasma ammonia levels to less than 200 μmol/L, less than 175 μmol/L, less than 150 μmol/L, less than 125 μmol/L, or less than 100 μmol/L, optionally wherein the reduced plasma ammonia levels are at 2 weeks, 4 weeks, 6 weeks, 8 weeks, 12 weeks, 16 weeks, 20 weeks, 6 months, 1 year, or 2 years after administering the combination. In some such methods, the method reduces plasma citrulline levels to less than 2000 μmol/L, less than 1750 μmol/L, less than 1500 μmol/L, less than 1250 μmol/L, less than 1000 μmol/L, less than 900 μmol/L, less than 800 μmol/L, less than 700 μmol/L, less than 600 μmol/L, or less than 500 μmol/L, optionally wherein the reduced plasma citrulline levels are at 2 weeks, 4 weeks, 6 weeks, 8 weeks, 12 weeks, 16 weeks, 20 weeks, 6 months, 1 year, or 2 years after administering the combination. In some such methods, the decreased ammonia and/or plasma citrulline levels are sustained for at least 1 month, at least 2 months, at least 3 months, at least 6 months, at least 1 year, or at least 2 years after administering the combination.

In some such methods, the method further comprises assessing preexisting AAV immunity in the subject prior to administering the composition to the subject. In some such methods, the preexisting AAV immunity is preexisting AAV8 immunity. In some such methods, the preexisting AAV immunity is preexisting AAV5 immunity. In some such methods, assessing preexisting AAV immunity comprises assessing immunogenicity using a total antibody immune assay or a neutralizing antibody assay.

### BRIEF DESCRIPTION OF THE FIGURES

FIG. 1 shows the experimental setup for an ASS1 episomal AAV efficacy study in adolescent P28 Ass1^{fold/fold} citrullinemia mice.

FIG. 2 shows body weight changes after treatment of Ass1^{fold/fold} mice with episomal human-ASS1-containing virus at various doses or control (GFP) virus.

FIG. 3 shows plasma ammonia changes after treatment of Ass1^{fold/fold} mice with episomal human-ASS1-containing virus at various doses or control (GFP) virus.

FIG. 4 shows plasma citrulline changes after treatment of Ass1^{fold/fold} mice with episomal human-ASS1-containing virus at various doses or control (GFP) virus.

FIG. 5 shows a mouse Ass1 intron 1 native locus gene insertion strategy in Ass1^{fold/fold} mice.

FIG. 6 shows the experimental setup for an ASS1 gene insertion efficacy study in adolescent P28 Ass1^{fold/fold} citrullinemia mice.

FIG. 7 shows body weight changes after treatment of P28 Ass1^{fold/fold} mice with AAV comprising human-ASS1 insertion template, alone or together with LNP comprising Cas9 mRNA and gRNA targeting mouse Ass1 intron 1.

FIGS. 8A and 8B shows plasma ammonia (FIG. 8A) and plasma citrulline (FIG. 8B) changes after treatment of P28 Ass1^{fold/fold} mice with AAV comprising human-ASS1 insertion template, alone or together with LNP comprising Cas9 mRNA and gRNA targeting mouse Ass1 intron 1. Measurements were performed the day before dosing and every 2 weeks after dosing.

FIG. 9 shows liver RT-qPCR for hybrid (mouse Ass1 exon1/human ASS1 exons 2-14) ASS1 gene expression after treatment of P28 Ass1^{fold/fold} mice with AAV comprising human-ASS1 insertion template, alone or together with LNP comprising Cas9 mRNA and gRNA targeting mouse Ass1 intron 1.

FIG. 10 shows droplet digital PCR (ddPCR) for hybrid DNA junctions of mouse Ass1 intron 1/insertion template derived Alb splice acceptor DNA in mouse liver genomic DNA, relative to the number of total mouse Ass1 alleles after treatment of P28 Ass1^{fold/fold} mice with AAV comprising human-ASS1 insertion template, alone or together with LNP comprising Cas9 mRNA and gRNA targeting mouse Ass1 intron 1.

FIG. 11 shows quantification of hepatocytes positive for hybrid (mouse/human fusion) ASS1 mRNA in situ hybridization (ISH) staining (BaseScope) after treatment of P28 Ass1^{fold/fold} mice with AAV comprising human-ASS1 insertion template, alone or together with LNP comprising Cas9 mRNA and gRNA targeting mouse Ass1 intron 1.

FIG. 12 shows the experimental setup for an ASS1 gene insertion efficacy study in neonatal P7 fold citrullinemia mice.

FIG. 13 shows body weight changes after treatment of P7 Ass1^{fold/fold} mice with AAV comprising human-ASS1 insertion template, alone or together with LNP comprising Cas9 mRNA and gRNA targeting mouse Ass1 intron 1.

FIGS. 14A and 14B shows plasma ammonia (FIG. 14A) and plasma citrulline (FIG. 14B) changes after treatment of P7 Ass1^{fold/fold} mice with AAV comprising human-ASS1 insertion template, alone or together with LNP comprising Cas9 mRNA and gRNA targeting mouse Ass1 intron 1.

FIG. 15 shows liver RT-qPCR for hybrid (mouse Ass1 exon1/human ASS1 exons 2-14) ASS1 gene expression after treatment of P7 Ass1^{fold/fold} mice with AAV comprising human-ASS1 insertion template, alone or together with LNP comprising Cas9 mRNA and gRNA targeting mouse Ass1 intron 1.

FIG. 16 shows droplet digital PCR (ddPCR) for hybrid DNA junctions of mouse Ass1 intron 1/insertion template derived Alb splice acceptor DNA in mouse liver genomic DNA, relative to the number of total mouse Ass1 alleles after treatment of P7 Ass1^{fold/fold} mice with AAV comprising human-ASS1 insertion template, alone or together with LNP comprising Cas9 mRNA and gRNA targeting mouse Ass1 intron 1.

FIG. 17 shows a schematic showing the insertion scheme for inserting a bidirectional human ASS1 insertion template (coding exons 3-14) into intron 2 of the human ASS1 genomic locus (between coding exons 2 and 3).

FIG. 18 shows the experimental setup for an ASS1-HiBiT gene insertion gRNA screen in primary human hepatocytes.

FIG. 19 shows human ASS1 intron 2 gRNAs display a range of insertion efficiencies in a primary human hepatocyte screen.

FIG. 20 shows human ASS1 intron 2 gRNAs display a range of editing efficiencies that do not always correlate to insertion efficiency in a primary human hepatocyte screen.

FIG. 21 shows that luminescence correlates between LNP doses within human ASS1 intron 2 guide RNA screens 1 and 2 in primary human hepatocytes.

FIG. 22 shows that luminescence correlates at tested LNP concentrations between human ASS1 intron 2 guide RNA screens 1 and 2 in primary human hepatocytes.

FIG. 23 shows that guide RNA editing correlates between human ASS1 intron 2 guide RNA screens 1 and 2 in primary human hepatocytes.

FIG. 24 shows a schematic showing the insertion scheme for inserting a bidirectional human ASS1 insertion template (coding exons 2-14) into intron 1 of the human ASS1 genomic locus (between coding exons 1 and 2).

FIG. 25 shows the experimental setup for an ASS1-HiBiT gene insertion gRNA screen in primary human hepatocytes.

FIG. 26 shows human ASS1 intron 1 gRNAs display a range of insertion efficiencies in a primary human hepatocyte screen.

FIG. 27 shows human ASS1 intron 1 gRNAs display a range of editing efficiencies that do not always correlate to insertion efficiency in a primary human hepatocyte screen.

FIG. 28 shows that luminescence correlates between LNP doses within human ASS1 intron 1 guide RNA screens 1 and 2 in primary human hepatocytes.

FIG. 29 shows that luminescence correlates at tested LNP concentrations between human ASS1 intron 1 guide RNA screens 1 and 2 in primary human hepatocytes.

FIG. 30 shows that guide RNA editing correlates between human ASS1 intron 1 guide RNA screens 1 and 2 in primary human hepatocytes.

FIG. 31 shows body weight changes, plasma ammonia changes, and plasma citrulline changes after treatment of neonatal Ass1^{fold/fold} mice with episomal human-ASS1-containing virus at various doses or control (GFP) virus.

FIG. 32 shows rates of native locus insertion in humanized ASS1 wild type mice treated with AAV comprising human-ASS1 insertion template together with 0.3 mpk LNP comprising Cas9 mRNA and G035840, G035869, or G035858 gRNA targeting human ASS1 intron 2.

FIGS. 33A-33B show all tested CpG-depleted ASS1 insertion templates yielded comparable ASS1-HiBiT expression in a primary human hepatocyte insertion assay when administered together with LNP comprising Cas9 mRNA and G035838 (FIG. 33A) or G035847 (FIG. 33B).

FIG. 34 shows all tested CpG-depleted ASS1 insertion templates yielded comparable ASS1-HiBiT expression in a primary human hepatocyte insertion assay when administered together with LNP comprising Cas9 mRNA and G035838.

FIG. 35 shows cryptic splicing analysis of 3 fully CpG-depleted ASS1 insertion templates compared to the original ASS1 insertion template for insertion into human ASS1 intron 2.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “genomically integrated” refers to a nucleic acid that has been introduced into a cell such that the nucleotide sequence integrates into the genome of the cell. Any protocol may be used for the stable incorporation of a nucleic acid into the genome of a cell.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells in vitro, ex vivo, or in vivo. Numerous forms of viral vectors are known.

The term “isolated” with respect to cells, tissues (e.g., liver samples), proteins, and nucleic acids includes cells, tissues (e.g., liver samples), proteins, and nucleic acids that are relatively purified with respect to other bacterial, viral, cellular, or other components that may normally be present in situ, up to and including a substantially pure preparation of the cells, tissues (e.g., liver samples), proteins, and nucleic acids. The term “isolated” also includes cells, tissues (e.g., liver samples), proteins, and nucleic acids that have no naturally occurring counterpart, have been chemically synthesized and are thus substantially uncontaminated by other cells, tissues (e.g., liver samples), proteins, and nucleic acids, or has been separated or purified from most other components (e.g., cellular components) with which they are naturally accompanied (e.g., other cellular proteins, polynucleotides, or cellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or animal. For example, an endogenous ASS1 sequence of a human refers to a native ASS1 sequence that naturally occurs at the ASS1 locus in the human.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” (i.e., “codon optimized” sequences) takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding an argininosuccinate synthase protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Res. 28(1):292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, an “ASS1 locus” may refer to the specific location of an ASS1 gene, ASS1 DNA sequence, argininosuccinate-synthase-encoding sequence, or ASS1 position on a chromosome of the genome of an organism that has been identified as to where such a sequence resides. An “ASS1 locus” may comprise a regulatory element of an ASS1 gene, including, for example, an enhancer, a promoter, 5′ and/or 3′ untranslated region (UTR), or a combination thereof.

The term “gene” refers to DNA sequences in a chromosome that may contain, if naturally present, at least one coding and at least one non-coding region. The DNA sequence in a chromosome that codes for a product (e.g., but not limited to, an RNA product and/or a polypeptide product) can include the coding region interrupted with non-coding introns and sequence located adjacent to the coding region on both the 5′ and 3′ ends such that the gene corresponds to the full-length mRNA (including the 5′ and 3′ untranslated sequences). Additionally, other non-coding sequences including regulatory sequences (e.g., but not limited to, promoters, enhancers, and transcription factor binding sites), polyadenylation signals, internal ribosome entry sites, silencers, insulating sequence, and matrix attachment regions may be present in a gene. These sequences may be close to the coding region of the gene (e.g., but not limited to, within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a mouse cell, a rat cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The methods and compositions provided herein employ a variety of different components. Some components throughout the description can have active variants and fragments. The term “functional” refers to the innate ability of a protein or nucleic acid (or a fragment or variant thereof) to exhibit a biological activity or function. The biological functions of functional fragments or variants may be the same or may in fact be changed (e.g., with respect to their specificity, selectivity, or efficacy) in comparison to the original molecule, but with retention of the molecule's basic biological function.

The term “variant” refers to a nucleotide sequence differing from the sequence most prevalent in a population (e.g., by one nucleotide) or a protein sequence different from the sequence most prevalent in a population (e.g., by one amino acid).

The term “fragment,” when referring to a protein, means a protein that is shorter or has fewer amino acids than the full-length protein. The term “fragment,” when referring to a nucleic acid, means a nucleic acid that is shorter or has fewer nucleotides than the full-length nucleic acid. A fragment can be, for example, when referring to a protein fragment, an N-terminal fragment (i.e., removal of a portion of the C-terminal end of the protein), a C-terminal fragment (i.e., removal of a portion of the N-terminal end of the protein), or an internal fragment (i.e., removal of a portion of each of the N-terminal and C-terminal ends of the protein). A fragment can be, for example, when referring to a nucleic acid fragment, a 5′ fragment (i.e., removal of a portion of the 3′ end of the nucleic acid), a 3′ fragment (i.e., removal of a portion of the 5′ end of the nucleic acid), or an internal fragment (i.e., removal of a portion each of the 5′ and 3′ ends of the nucleic acid).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube or an isolated cell or cell line). The term “in vivo” includes natural environments (e.g., a cell, organism, or body) and to processes or reactions that occur within a natural environment. The term “ex vivo” includes cells that have been removed from the body of an individual and processes or reactions that occur within such cells.

As used herein, the term “neonatal” in the context of humans covers human subjects up to or under the age of 1 year (52 weeks), preferably up to or under the age of 24 weeks, more preferably up to or under the age of 12 weeks, more preferably up to or under the age of 8 weeks, even more preferably up to or under the age of 4 weeks, even more preferably up to or under the age of 2 weeks, and even more preferably up to or under the age of 1 week. In certain embodiments, a neonatal human subject is up to 1 week of age. In certain embodiments, a neonatal human subject is up to 2 weeks of age. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject.

As used herein, a “control” as in a control sample or a control subject is a comparator for a measurement, e.g., a diagnostic measurement of a sign or symptom of a disease. In certain embodiments, a control can be a subject sample from the same subject an earlier time point, e.g., before a treatment intervention. In certain embodiments, a control can be a measurement from a normal subject, i.e., a subject not having the disease of the treated subject, to provide a normal control, e.g., argininosuccinate-synthase activity in a subject sample. In certain embodiments, a normal control can be a population control, i.e., the average of subjects in the general population. In certain embodiments, a control can be an untreated subject with the same disease. In certain embodiments, a control can be a subject treated with a different therapy, e.g., the standard of care. In certain embodiments, a control can be a subject or a population of subjects from a natural history study of subjects with the disease of the subject being compared. In certain embodiments, the control is matched for certain factors to the subject being tested, e.g., age, gender. In certain embodiments, a control may be a control level for a particular lab, e.g., a clinical lab. Selection of an appropriate control is within the ability of those of skill in the art.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing an upper limit even if one is not specifically provided as it would be clearly understood. Similarly, up to 3 nucleotides would be understood to encompass 0, 1, 2, or 3 nucleotides, providing a lower limit even if one is not specifically provided. When “at least,” “up to,” or other similar language modifies a number, it can be understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

Unless otherwise apparent from the context, the term “about” encompasses values 5% of a stated value. In certain embodiments, the term “about” is understood to encompass tolerated variation or error within the art, e.g., 2 standard deviations from the mean, or the sensitivity of the method used to take a measurement, or a percent of a value as tolerated in the art, e.g., with age. When “about” is present before the first value of a series, it can be understood to modify each value in the series.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

In the event of a conflict between a sequence in the application and an indicated accession number or position in an accession number, the sequence in the application predominates.

### DETAILED DESCRIPTION

### I. Overview

Provided herein are nucleic acid constructs and compositions that allow insertion of an argininosuccinate synthase 1 (ASS1) coding sequence into a target genomic locus such as an endogenous ASS1 locus and/or expression of the ASS1 coding sequence. The nucleic acid constructs and compositions can be used in methods of introducing an ASS1 nucleic acid into a cell, in methods of integration of an ASS1 nucleic acid into a target genomic locus, in methods of expression of ASS1 in a cell, and in methods of treating citrullinemia type I or ASS1 deficiency in a subject. In some cases, the cells or subjects can be neonatal cells or neonatal subjects as defined herein. In other cases, the cells are not neonatal cells, and the subjects are not neonatal subjects. Also provided are nuclease agents (e.g., targeting an endogenous ASS1 locus) or nucleic acids encoding nuclease agents to facilitate integration of the nucleic acid constructs into a target genomic locus such as an endogenous ASS1 locus.

Also provided are compositions, combinations, or kits comprising an ASS1 nucleic acid construct in combination with a nuclease agent or one or more nucleic acids encoding the nuclease agent, wherein the nuclease agent targets a nuclease target site in a target genomic locus (e.g., an endogenous ASS1 locus). As used herein, the term “in combination with” means that additional component(s) may be administered prior to, concurrent with, or after the administration of the nucleic acid construct. The different components of the combination can be formulated into a single composition, e.g., for simultaneous delivery, or formulated separately into two or more compositions (e.g., a kit including each component, for example, wherein the further agent is in a separate formulation).

More specifically, described herein in some embodiments is a therapeutic product based on the CRISPR/Cas9 gene editing technology and optionally contained in a lipid nanoparticle (LNP) delivery system, associated with an ASS1 DNA gene insertion template (e.g., a unidirectional or bidirectional ASS1 DNA gene insertion template) optionally contained in a recombinant adeno-associated virus serotype 8 (rAAV8) or recombinant adeno-associated virus serotype 5 (rAAV5). The CRISPR/Cas9 component has been designed to target and cut the double stranded DNA at a target gene locus (e.g., an endogenous ASS1 gene locus in hepatocytes), allowing for the ASS1 DNA template to be inserted in the genome at the target genomic locus. Transgene insertion provides a functional ASS1 gene that when expressed provides functional ASS1 to rescue the defective endogenous, genomic ASS1 in citrullinemia type I patients.

In some cases, the ASS1 coding sequences in the constructs disclosed herein are optimized for expression as compared to native ASS1 coding sequence. In other cases, the ASS1 coding sequences in the constructs disclosed herein can comprise native ASS1 coding sequences. For example, the ASS1 coding sequences in the constructs disclosed herein may include one or more modifications such as codon optimization (e.g., to human codons), depletion of CpG dinucleotides, mutation of cryptic splice sites, or any combination thereof.

In particular, provided herein are bidirectional constructs comprising two different ASS1 coding sequences (i.e., a first ASS1 coding sequence and a reverse complement of a second ASS1 coding sequence). Also provided herein are unidirectional constructs comprising a single ASS1 coding sequence.

In some embodiments, the gene insertion platform described herein also has advantages over existing episomal ASS1 platforms. For example, there are no concerns regarding integration of promoter-containing constructs because the ASS1 insertion templates used were promoterless. In some embodiments, because there are no promoter/regulatory elements in the AAV cassettes used, they are less likely to influence expression of neighboring loci if randomly inserted. Likewise, in some embodiments, because there is no in-frame ATG/methionine at the 5′ end of the cassettes used, no protein is produced from the template alone. In addition, there is an extremely low likelihood of producing protein from off-target insertion because it would require intronic insertion leading to splicing into the correct reading frame. Moreover, integration of the coding sequence as in the compositions and methods disclosed herein is advantageous over non-integrating episomal vectors because transgene retention over time can be problematic with non-replicating episomal vectors, making it necessary to administer more virus for continued therapeutic response. However, these subsequent exposures may result in rapid neutralization of the virus and, therefore, decreased transgene expression. In some embodiments, no redosing is required because the compositions and methods result in integration into the genome and permanent expression. A major advantage that ASS1 insertion affords over episomal expression is that it enables durable expression in the face of cellular division, whereas episomal expression is largely lost upon cell division. This is particularly relevant for citrullinemia type I where there is a need for early life intervention. Episome expression approaches are largely ineffective when delivered early in life because the liver is still growing and there is significant cell division. In short, ASS1 insertion enables treating young individuals with citrullinemia, whereas ASS1 episomal expression approaches do not. This is medically very important as cognitive and motor deficits that accumulate over time in citrullinemia due to insufficient control of plasma ammonia levels are largely irreversible. Another advantage associated with ASS1 native locus insertion is that the insertion template packaged in the AAV capsid lacks a promoter. It is increasingly accepted that AAV treatment leads to a low level of random genomic integration of the recombinant AAV genome. AAV episome gene expression cassettes invariably contain promoter/enhancer elements to enable transgene expression. When such promoter/enhancer elements randomly integrate into the genome, there is a possibility that they can lead to mis-regulation of a neighboring gene, which can have unintended negative consequences. The promoter-less nature of the ASS1 insertion template provides an added safety feature in that it is devoid of such promoter/enhancer elements.

The native locus gene insertion platform described herein also has advantages over insertion platforms in which an ASS1 transgene is inserted into a non-ASS1 genomic locus. Native locus insertion ensures ASS1 insertion transgene is expressed in the proper amount because expression is regulated by the native locus promoter. Insertion into another locus such as an ALB locus would lead to dramatic overexpression of ASS1 protein, with unknown consequences. Native locus insertion may also ensure proper cytoplasmic localization of ASS1 protein. Insertion of ASS1 into another locus such as an ALB locus would likely lead to some fraction of ASS1 protein being mis-localized into unintended cellular compartments, such as endoplasmic reticulum, secretory vesicles, and extracellular space, because ALB exon 1 encodes an ALB signal peptide. Native locus insertion may also ensure proper regulation of ASS1 in response to physiological queues. Insertion into another locus such as an ALB locus would subject ASS1 expression to the acute phase response, which would lead to downregulation of ASS1 expression under inflammatory and stress responses. Insertion into a non-ASS1 genomic locus would also be difficult to achieve without significantly impacting expression of the endogenous protein from the locus. For instance, insertion into an early intron of a non-ASS1 genomic locus would likely lead to ablation of expression of the endogenous protein from the targeted allele. Insertion into a downstream intron would likely lead to epitope tagging of endogenous protein.

### II. Compositions for Expressing Argininosuccinate Synthase

Provided herein are nucleic acid constructs and compositions that allow insertion of an argininosuccinate synthase (ASS1) coding sequence into a target genomic locus such as an endogenous ASS1 locus and/or expression of the ASS1 coding sequence. The nucleic acid constructs and compositions can be used in methods for integration into a target genomic locus and/or expression in a cell or in methods of treating citrullinemia type I (CTLN1) or ASS1 deficiency. Also provided are nuclease agents (e.g., targeting an endogenous ASS1 locus) or nucleic acids encoding nuclease agents to facilitate integration of the nucleic acid constructs into a target genomic locus such as an endogenous ASS1 locus.

### A. Argininosuccinate Synthase Nucleic Acid Constructs

The compositions and methods described herein include the use of a nucleic acid construct that comprises an ASS1 protein coding sequence (an ASS1 nucleic acid) or a reverse complement of the ASS1 protein coding sequence (e.g., a heterologous ASS1 protein coding sequence (a heterologous ASS1 nucleic acid) or a reverse complement of the heterologous ASS1 protein coding sequence). For example, the nucleic acid construct can comprise an ASS1 protein coding sequence (an ASS1 nucleic acid), such as a heterologous ASS1 protein coding sequence (e.g., a heterologous ASS1 nucleic acid). For example, the ASS1 protein coding sequence can comprise a human ASS1 (hASS1) protein coding sequence comprising exons 3-14 of hASS1 (e.g., encoding the amino acid sequence set forth in SEQ ID NO: 230). As another example, the ASS1 protein coding sequence can comprise a hASS1 protein coding sequence comprising exons 2-14 of hASS1 (e.g., encoding the amino acid sequence set forth in SEQ ID NO: 227). As another example, the ASS1 protein coding sequence can comprise the complete hASS1 protein coding sequence (e.g., encoding the amino acid sequence set forth in SEQ ID NO: 1). Such nucleic acid constructs can be for insertion into a target genomic locus following cleavage by a nuclease agent or CRISPR/Cas system, as disclosed elsewhere herein, or can be for expression of ASS1 without insertion into a target genomic locus (e.g., in an episome). For example, such nucleic acid constructs can be for insertion into a cleavage site created by a nuclease agent or CRISPR/Cas system, as disclosed elsewhere herein, or can be for expression of ASS1 without insertion into a cleavage site (e.g., in an episome). The term cleavage site includes a DNA sequence at which a nick or double-strand break is created by a nuclease agent (e.g., a Cas9 protein complexed with a guide RNA). A “heterologous” ASS1 protein coding sequence can refer to a coding sequence that has been introduced as an exogenous source to a site within a host cell genome (e.g., at a genomic locus such as the endogenous ASS1 locus, including human ASS1 intron 2, human ASS1 intron 1, or mouse Ass1 intron 1). That is, the heterologous protein coding sequence can be heterologous with respect to its insertion site, and the polypeptide expressed from such a heterologous coding sequence is referred to as a heterologous polypeptide. Additional sites within a host cell genome for introduction of a heterologous ASS1 protein coding sequence can comprise a genomic safe harbor locus, including human albumin (ALB) or mouse Alb. The heterologous coding sequence can be naturally-occurring or engineered, and can be wild type or a variant. The heterologous coding sequence may include nucleotide sequences other than the sequence that encodes the heterologous polypeptide (e.g., an internal ribosomal entry site). The heterologous coding sequence can be a coding sequence that occurs naturally in the host genome, as a wild type or a variant (e.g., mutant). For example, although the host cell contains the coding sequence of interest (as a wild type or as a variant), the same coding sequence or variant thereof can be introduced as an exogenous source (e.g., for expression at a locus that is highly expressed). The heterologous coding sequence can also be a coding sequence that is not naturally occurring in the host genome, or that expresses a heterologous polypeptide that does not naturally occur in the host genome. A heterologous coding sequence can include an exogenous nucleic acid sequence (e.g., a nucleic acid sequence is not endogenous to the recipient cell), or may be heterologous with respect to its insertion site and/or with respect to its recipient cell.

The length of the ASS1 nucleic acid constructs disclosed herein can vary. The construct can be, for example, from about 1 kb to about 5 kb, such as from about 1 kb to about 4.5 kb or about 1 kb to about 4 kb. An exemplary nucleic acid construct is between about 1 kb to about 5 kb in length or between about 1 kb to about 4 kb in length. Alternatively, a nucleic acid construct can be between about 1 kb to about 1.5 kb, about 1.5 kb to about 2 kb, about 2 kb to about 2.5 kb, about 2.5 kb to about 3 kb, about 3 kb to about 3.5 kb, about 3.5 kb to about 4 kb, about 4 kb to about 4.5 kb, or about 4.5 kb to about 5 kb in length. Alternatively, a nucleic acid construct can be, for example, no more than 5 kb, no more than 4.5 kb, no more than 4 kb, no more than 3.5 kb, no more than 3 kb, or no more than 2.5 kb in length. In a specific example, the nucleic acid construct is no more than 3.5 kb in length.

The constructs can comprise deoxyribonucleic acid (DNA) or ribonucleic acid (RNA), can be single-stranded, double-stranded, or partially single-stranded and partially double-stranded, and can be introduced into a host cell in linear or circular (e.g., minicircle) form. See, e.g., US 2010/0047805, US 2011/0281361, and US 2011/0207221, each of which is herein incorporated by reference in their entirety for all purposes. If introduced in linear form, the ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in their entirety for all purposes. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. A construct can be introduced into a cell as part of a vector molecule having additional sequences such as, for example, replication origins, promoters, and genes encoding antibiotic resistance. A construct may omit viral elements. Moreover, constructs can be introduced as a naked nucleic acid, can be introduced as a nucleic acid complexed with an agent such as a liposome or poloxamer, or can be delivered by viruses (e.g., adenovirus, adeno-associated virus (AAV), herpesvirus, retrovirus, or lentivirus).

The constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Some constructs may be inserted so that their expression is driven by the endogenous promoter at the insertion site (e.g., the endogenous ASS1 promoter when the construct is integrated into the host cell's ASS1 locus). Such constructs may not comprise a promoter that drives the expression of ASS1. For example, the expression of ASS1 can be driven by a promoter of the host cell (e.g., the endogenous ASS1 promoter when the transgene is integrated into a host cell's ASS1 locus). In such cases, the construct may lack control elements (e.g., promoter and/or enhancer) that drive its expression (e.g., a promoterless construct). Nonetheless, in other cases the construct may comprise a promoter and/or enhancer, for example, a constitutive promoter or an inducible or tissue-specific (e.g., liver-specific) promoter that drives expression of the ASS1 in an episome or upon integration. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. For example, the promoter may be a CMV promoter or a truncated CMV promoter. In another example, the promoter may be an EF1a promoter. Non-limiting exemplary inducible promoters include those inducible by heat shock, light, chemicals, peptides, metals, steroids, antibiotics, or alcohol. The inducible promoter may be one that has a low basal (non-induced) expression level, such as the Tet-On® promoter (Clontech). Although not required for expression, the constructs may comprise transcriptional or translational regulatory sequences such as promoters, enhancers, insulators, internal ribosome entry sites, additional sequences encoding peptides, and/or polyadenylation signals. In some examples, the nucleic acid construct works in homology-independent insertion of a nucleic acid that encodes an ASS1 protein. Such nucleic acid constructs can work, for example, in non-dividing cells (e.g., cells in which non-homologous end joining (NHEJ), not homologous recombination (HR), is the primary mechanism by which double-stranded DNA breaks are repaired). Such constructs can be, for example, homology-independent donor constructs. Such nucleic acid constructs can work, for example, in dividing cells (e.g., actively dividing cells).

The constructs disclosed herein can be modified to include or exclude any suitable structural feature as needed for any particular use and/or that confers one or more desired function. For example, some constructs disclosed herein do not comprise a homology arm. Some constructs disclosed herein are capable of insertion into a cut site in a target DNA sequence for a nuclease agent (e.g., capable of insertion into the endogenous ASS1 locus, such as into human ASS1 intron 2, human ASS1 intron 1, or mouse Ass1 intron 1) by non-homologous end joining. Some such constructs do not comprise homology arms. For example, such constructs can be inserted into a blunt end double-strand break following cleavage with a nuclease agent (e.g., CRISPR/Cas system) as disclosed herein. In a specific example, the construct can be delivered via AAV and can be capable of insertion by non-homologous end joining (e.g., the construct can be one that does not comprise homology arms).

In a particular example, the construct can be inserted via homology-independent targeted integration. For example, the heterologous ASS1 nucleic acid in the construct can be flanked on each side by a target site for a nuclease agent (e.g., the same target site as in the target DNA sequence for targeted insertion (e.g., in the endogenous ASS1 locus, such as in human ASS1 intron 2, human ASS1 intron 1, or mouse Ass1 intron 1), and the same nuclease agent being used to cleave the target DNA sequence for targeted insertion). The nuclease agent can then cleave the target sites flanking the heterologous ASS1 nucleic acid. In a specific example, the construct is delivered AAV-mediated delivery, and cleavage of the target sites flanking the heterologous ASS1 nucleic acid can remove the inverted terminal repeats (ITRs) of the AAV. In some instances, the target DNA sequence for targeted insertion (e.g., target DNA sequence in a safe harbor locus such as a gRNA target sequence including the flanking protospacer adjacent motif) is no longer present if the heterologous ASS1 nucleic acid is inserted into the cut site or target DNA sequence in the correct orientation but it is reformed if the heterologous ASS1 nucleic acid is inserted into the cut site or target DNA sequence in the opposite orientation. This can help ensure that the heterologous ASS1 nucleic acid is inserted in the correct orientation for expression.

The constructs disclosed herein can comprise a polyadenylation tail sequence (e.g., downstream or 3′ of an ASS1 coding sequence). Methods of designing a suitable polyadenylation tail sequence are well-known. The polyadenylation tail sequence can be encoded, for example, as a “poly-A” stretch downstream of the ASS1 coding sequence. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes. The term polyadenylation signal sequence refers to any sequence that directs termination of transcription and addition of a poly-A tail to the mRNA transcript. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. In one example, the polyadenylation signal is a simian virus 40 (SV40) late polyadenylation signal. For example, the polyadenylation signal can comprise, consist essentially of, or consist of SEQ ID NO: 239 or SEQ ID NO: 240. In another example, the polyadenylation signal is a bovine growth hormone (BGH) polyadenylation signal. For example, the polyadenylation signal can comprise, consist essentially of, or consist of SEQ ID NO: 236, SEQ ID NO: 237, or SEQ ID NO: 238. In another example, the polyadenylation signal is a human growth hormone (HGH) polyadenylation signal. For example, the polyadenylation signal can comprise, consist essentially of, or consist of SEQ ID NO: 235.

The constructs disclosed herein may also comprise splice acceptor sites (e.g., operably linked to the ASS1 coding sequence, such as upstream or 5′ of the ASS1 coding sequence). The splice acceptor site can, for example, comprise NAG or consist of NAG. In a specific example, the splice acceptor is an ASS1 splice acceptor (e.g., an ASS1 splice acceptor used in the splicing together of exons 2 and 3 of ASS1 (i.e., ASS1 exon 3 splice acceptor) or an ASS1 splice acceptor used in the splicing together of exons 1 and 3 of ASS1 (i.e., ASS1 exon 2 splice acceptor). For example, such a splice acceptor can be derived from the human ASS1 gene (e.g., an ASS1 splice acceptor used in the splicing together of exons 2 and 3 of human ASS1 (i.e., human ASS1 exon 3 splice acceptor)). In another example, the splice acceptor can be derived from the mouse Ass1 gene (e.g., an ASS1 splice acceptor used in the splicing together of exons 1 and 2 of mouse Ass1 (i.e., mouse Ass1 exon 2 splice acceptor)). In another example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). Additional suitable splice acceptor sites useful in eukaryotes, including artificial splice acceptors, are well-known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes. In a specific example, the splice acceptor is a human ASS1 exon 3 splice acceptor. In another example, the splice acceptor is a mouse Ass1 exon 2 splice acceptor. In another example, the splice acceptor is a mouse Alb exon 2 splice acceptor. In a specific example, the splice acceptor can comprise, consist essentially of, or consist of SEQ ID NO: 248.

In some examples, the nucleic acid constructs disclosed herein can be bidirectional constructs, which are described in more detail below. In some examples, the nucleic acid constructs disclosed herein can be unidirectional constructs, which are described in more detail below. Likewise, in some examples, the nucleic acid constructs disclosed herein can be in a vector (e.g., viral vector, such as AAV, or rAAV8 or rAAV5) and/or a lipid nanoparticle as described in more detail elsewhere herein.

### (1) Argininosuccinate Synthase (ASS1)

Argininosuccinate synthase 1 (ASS1) is encoded by ASS1 and is a 412-amino acid enzyme present in the cytoplasm of most tissues. ASS1 is one of the enzymes of the urea cycle, the metabolic pathway responsible for transforming neurotoxic ammonia produced by protein catabolism into urea in the liver of ureotelic animals. Specifically, ASS1 catalyzes the formation of arginosuccinate from aspartate, citrulline, and ATP. Together with argininosuccinate lyase, it is responsible for the biosynthesis of arginine in most body tissues.

The ASS1 expressed from the compositions and methods disclosed herein can be any wild type or variant ASS1. In one example, the ASS1 is a human ASS1 protein. Human ASS1 is assigned UniProt reference number P00966. An exemplary amino acid sequence for human ASS1 is assigned NCBI Accession No. NP_000041.2 and is set forth in SEQ ID NO: 1. An exemplary human ASS1 mRNA (cDNA) sequence is assigned NCBI Accession No. NM_000050.4 and is set forth in SEQ ID NO: 2. An exemplary human ASS1 coding sequence is assigned CCDS ID CCDS6933.1 and is set forth in SEQ ID NO: 3.

In some examples, the ASS1 (e.g., human ASS1) is a wild type ASS1 (e.g., wild type human ASS1) sequence or a fragment thereof. In a specific example, the ASS1 encoded by the nucleic acid constructs disclosed herein can comprise SEQ ID NO: 230, can consist of SEQ ID NO: 230, or can be at least 90%, at least 91%, at least 92%, at least 93%, at least 94%, at least 95%, at least 96%, at least 97%, at least 98%, at least 99%, or at least 99.5% identical to SEQ ID NO: 230 (encoded by exons 3-14 of human ASS1). In another example, the ASS1 encoded by the nucleic acid constructs disclosed herein can comprise SEQ ID NO: 227, can consist of SEQ ID NO: 227, or can be at least 90%, at least 91%, at least 92%, at least 93%, at least 94%, at least 95%, at least 96%, at least 97%, at least 98%, at least 99%, or at least 99.5% identical to SEQ ID NO: 227 (encoded by exons 2-14 of human ASS1). In another example, the ASS1 encoded by the nucleic acid constructs disclosed herein can comprise SEQ ID NO: 1, can consist of SEQ ID NO: 1, or can be at least 90%, at least 91%, at least 92%, at least 93%, at least 94%, at least 95%, at least 96%, at least 97%, at least 98%, at least 99%, or at least 99.5% identical to SEQ ID NO: 1 (encoded by complete human ASS1 CDS).

The ASS1 coding sequences in the constructs disclosed herein may include wild type ASS1 coding sequences without any modifications. Alternatively, the ASS1 coding sequences may be optimized. The ASS1 coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. For example, the ASS1 coding sequences can be CpG-depleted (e.g., fully CpG depleted) and/or codon optimized and/or modified to remove cryptic splice sites.

In one example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 229. In another example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 229 and encodes an ASS1 protein (or an ASS1 protein comprising a sequence) at least 99%, at least 99.5%, or 100% identical to SEQ ID NO: 230. In another example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 229 and encodes an ASS1 protein comprising the sequence set forth in SEQ ID NO: 230. In another example, the ASS1 coding sequence is (or comprises a sequence) at least 95%, at least 96%, at least 97%, at least 98%, at least 99%, at least 99.5%, or 100% identical to SEQ ID NO: 229. In another example, the ASS1 coding sequence is (or comprises a sequence) at least 95%, at least 96%, at least 97%, at least 98%, at least 99%, at least 99.5%, or 100% identical to SEQ ID NO: 229 and encodes an ASS1 protein (or an ASS1 protein comprising a sequence) at least 99%, at least 99.5%, or 100% identical to SEQ ID NO: 230. In another example, the ASS1 coding sequence is (or comprises a sequence) at least 95%, at least 96%, at least 97%, at least 98%, at least 99%, at least 99.5%, or 100% identical to SEQ ID NO: 229 and encodes an ASS1 protein comprising the sequence set forth in SEQ ID NO: 230. In another example, the ASS1 coding sequence is (or comprises a sequence) at least 99%, at least 99.5%, or 100% identical to SEQ ID NO: 229. In another example, the ASS1 coding sequence is (or comprises a sequence) at least 99%, at least 99.5%, or 100% identical to SEQ ID NO: 229 and encodes an ASS1 protein (or an ASS1 protein comprising a sequence) at least 99%, at least 99.5%, or 100% identical to SEQ ID NO: 230. In another example, the ASS1 coding sequence is (or comprises a sequence) at least 99%, at least 99.5%, or 100% identical to SEQ ID NO: 229 and encodes an ASS1 protein comprising the sequence set forth in SEQ ID NO: 230. In another example, the ASS1 coding sequence comprises the sequence set forth in SEQ ID NO: 229. In another example, the ASS1 coding sequence consists essentially of the sequence set forth in SEQ ID NO: 229. In another example, the ASS1 coding sequence consists of the sequence set forth in SEQ ID NO: 229. Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230.

In one example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 228. In another example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 228 and encodes an ASS1 protein (or an ASS1 protein comprising a sequence) at least 99%, at least 99.5%, or 100% identical to SEQ ID NO: 230. In another example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 228 and encodes an ASS1 protein comprising the sequence set forth in SEQ ID NO: 230. In another example, the ASS1 coding sequence is (or comprises a sequence) at least 95%, at least 96%, at least 97%, at least 98%, at least 99%, at least 99.5%, or 100% identical to SEQ ID NO: 228. In another example, the ASS1 coding sequence is (or comprises a sequence) at least 95%, at least 96%, at least 97%, at least 98%, at least 99%, at least 99.5%, or 100% identical to SEQ ID NO: 228 and encodes an ASS1 protein (or an ASS1 protein comprising a sequence) at least 99%, at least 99.5%, or 100% identical to SEQ ID NO: 230. In another example, the ASS1 coding sequence is (or comprises a sequence) at least 95%, at least 96%, at least 97%, at least 98%, at least 99%, at least 99.5%, or 100% identical to SEQ ID NO: 228 and encodes an ASS1 protein comprising the sequence set forth in SEQ ID NO: 230. In another example, the ASS1 coding sequence is (or comprises a sequence) at least 99%, at least 99.5%, or 100% identical to SEQ ID NO: 228. In another example, the ASS1 coding sequence is (or comprises a sequence) at least 99%, at least 99.5%, or 100% identical to SEQ ID NO: 228 and encodes an ASS1 protein (or an ASS1 protein comprising a sequence) at least 99%, at least 99.5%, or 100% identical to SEQ ID NO: 230. In another example, the ASS1 coding sequence is (or comprises a sequence) at least 99%, at least 99.5%, or 100% identical to SEQ ID NO: 228 and encodes an ASS1 protein comprising the sequence set forth in SEQ ID NO: 230. In another example, the ASS1 coding sequence comprises the sequence set forth in SEQ ID NO: 228. In another example, the ASS1 coding sequence consists essentially of the sequence set forth in SEQ ID NO: 228. In another example, the ASS1 coding sequence consists of the sequence set forth in SEQ ID NO: 228. Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230.

In one example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 706 and 710 (optionally encoding an ASS1 protein comprising the sequence set forth in SEQ ID NO: 230). In another example, the ASS1 coding sequence is (or comprises a sequence) at least 95%, at least 96%, at least 97%, at least 98%, at least 99%, at least 99.5%, or 100% identical to any one of SEQ ID NOS: 706 and 710 (optionally encoding an ASS1 protein comprising the sequence set forth in SEQ ID NO: 230). In another example, the ASS1 coding sequence is (or comprises a sequence) at least 99%, at least 99.5%, or 100% identical to any one of SEQ ID NOS: 706 and 710 (optionally encoding an ASS1 protein comprising the sequence set forth in SEQ ID NO: 230). In another example, the ASS1 coding sequence comprises the sequence set forth in any one of SEQ ID NOS: 706 and 710 (optionally encoding an ASS1 protein comprising the sequence set forth in SEQ ID NO: 230). In another example, the ASS1 coding sequence consists essentially of the sequence set forth in any one of SEQ ID NOS: 706 and 710 (optionally encoding an ASS1 protein comprising the sequence set forth in SEQ ID NO: 230). In another example, the ASS1 coding sequence consists of the sequence set forth in any one of SEQ ID NOS: 706 and 710 (optionally encoding an ASS1 protein comprising the sequence set forth in SEQ ID NO: 230). Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230 (i.e., protein encoded by exons 3-14 of human ASS1).

In one example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 706 (optionally encoding an ASS1 protein comprising the sequence set forth in SEQ ID NO: 230). In another example, the ASS1 coding sequence is (or comprises a sequence) at least 95%, at least 96%, at least 97%, at least 98%, at least 99%, at least 99.5%, or 100% identical to SEQ ID NO: 706 (optionally encoding an ASS1 protein comprising the sequence set forth in SEQ ID NO: 230). In another example, the ASS1 coding sequence is (or comprises a sequence) at least 99%, at least 99.5%, or 100% identical to SEQ ID NO: 706 (optionally encoding an ASS1 protein comprising the sequence set forth in SEQ ID NO: 230). In another example, the ASS1 coding sequence comprises the sequence set forth in SEQ ID NO: 706 (optionally encoding an ASS1 protein comprising the sequence set forth in SEQ ID NO: 230). In another example, the ASS1 coding sequence consists essentially of the sequence set forth in SEQ ID NO: 706 (optionally encoding an ASS1 protein comprising the sequence set forth in SEQ ID NO: 230). In another example, the ASS1 coding sequence consists of the sequence set forth in SEQ ID NO: 706 (optionally encoding an ASS1 protein comprising the sequence set forth in SEQ ID NO: 230). Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230 (i.e., protein encoded by exons 3-14 of human ASS1).

In one example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 710 (optionally encoding an ASS1 protein comprising the sequence set forth in SEQ ID NO: 230). In another example, the ASS1 coding sequence is (or comprises a sequence) at least 95%, at least 96%, at least 97%, at least 98%, at least 99%, at least 99.5%, or 100% identical to SEQ ID NO: 710 (optionally encoding an ASS1 protein comprising the sequence set forth in SEQ ID NO: 230). In another example, the ASS1 coding sequence is (or comprises a sequence) at least 99%, at least 99.5%, or 100% identical to SEQ ID NO: 710 (optionally encoding an ASS1 protein comprising the sequence set forth in SEQ ID NO: 230). In another example, the ASS1 coding sequence comprises the sequence set forth in SEQ ID NO: 710 (optionally encoding an ASS1 protein comprising the sequence set forth in SEQ ID NO: 230). In another example, the ASS1 coding sequence consists essentially of the sequence set forth in SEQ ID NO: 710 (optionally encoding an ASS1 protein comprising the sequence set forth in SEQ ID NO: 230). In another example, the ASS1 coding sequence consists of the sequence set forth in SEQ ID NO: 710 (optionally encoding an ASS1 protein comprising the sequence set forth in SEQ ID NO: 230). Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230 (i.e., protein encoded by exons 3-14 of human ASS1).

In one example, the ASS1 coding sequence is (or comprises a sequence) at least 95%, at least 96%, at least 97%, at least 98%, at least 99%, at least 99.5%, or 100% identical to any one of SEQ ID NOS: 705, 709, and 713 (optionally encoding an ASS1 protein comprising the sequence set forth in SEQ ID NO: 230). In another example, the ASS1 coding sequence is (or comprises a sequence) at least 96%, at least 97%, at least 98%, at least 99%, at least 99.5%, or 100% identical to any one of SEQ ID NOS: 705, 709, and 713 (optionally encoding an ASS1 protein comprising the sequence set forth in SEQ ID NO: 230). In another example, the ASS1 coding sequence is (or comprises a sequence) at least 99%, at least 99.5%, or 100% identical to any one of SEQ ID NOS: 705, 709, and 713 (optionally encoding an ASS1 protein comprising the sequence set forth in SEQ ID NO: 230). In another example, the ASS1 coding sequence comprises the sequence set forth in any one of SEQ ID NOS: 705, 709, and 713 (optionally encoding an ASS1 protein comprising the sequence set forth in SEQ ID NO: 230). In another example, the ASS1 coding sequence consists essentially of the sequence set forth in any one of SEQ ID NOS: 705, 709, and 713 (optionally encoding an ASS1 protein comprising the sequence set forth in SEQ ID NO: 230). In another example, the ASS1 coding sequence consists of the sequence set forth in any one of SEQ ID NOS: 705, 709, and 713 (optionally encoding an ASS1 protein comprising the sequence set forth in SEQ ID NO: 230). Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230 (i.e., protein encoded by exons 3-14 of human ASS1).

In one example, the ASS1 coding sequence is (or comprises a sequence) at least 95%, at least 96%, at least 97%, at least 98%, at least 99%, at least 99.5%, or 100% identical to SEQ ID NO: 705 (optionally encoding an ASS1 protein comprising the sequence set forth in SEQ ID NO: 230). In another example, the ASS1 coding sequence is (or comprises a sequence) at least 96%, at least 97%, at least 98%, at least 99%, at least 99.5%, or 100% identical to SEQ ID NO: 705 (optionally encoding an ASS1 protein comprising the sequence set forth in SEQ ID NO: 230). In another example, the ASS1 coding sequence is (or comprises a sequence) at least 99%, at least 99.5%, or 100% identical to SEQ ID NO: 705 (optionally encoding an ASS1 protein comprising the sequence set forth in SEQ ID NO: 230). In another example, the ASS1 coding sequence comprises the sequence set forth in SEQ ID NO: 705 (optionally encoding an ASS1 protein comprising the sequence set forth in SEQ ID NO: 230). In another example, the ASS1 coding sequence consists essentially of the sequence set forth in SEQ ID NO: 705 (optionally encoding an ASS1 protein comprising the sequence set forth in SEQ ID NO: 230). In another example, the ASS1 coding sequence consists of the sequence set forth in SEQ ID NO: 705 (optionally encoding an ASS1 protein comprising the sequence set forth in SEQ ID NO: 230). Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230 (i.e., protein encoded by exons 3-14 of human ASS1).

In one example, the ASS1 coding sequence is (or comprises a sequence) at least 95%, at least 96%, at least 97%, at least 98%, at least 99%, at least 99.5%, or 100% identical to SEQ ID NO: 709 (optionally encoding an ASS1 protein comprising the sequence set forth in SEQ ID NO: 230). In another example, the ASS1 coding sequence is (or comprises a sequence) at least 96%, at least 97%, at least 98%, at least 99%, at least 99.5%, or 100% identical to SEQ ID NO: 709 (optionally encoding an ASS1 protein comprising the sequence set forth in SEQ ID NO: 230). In another example, the ASS1 coding sequence is (or comprises a sequence) at least 99%, at least 99.5%, or 100% identical to SEQ ID NO: 709 (optionally encoding an ASS1 protein comprising the sequence set forth in SEQ ID NO: 230). In another example, the ASS1 coding sequence comprises the sequence set forth in SEQ ID NO: 709 (optionally encoding an ASS1 protein comprising the sequence set forth in SEQ ID NO: 230). In another example, the ASS1 coding sequence consists essentially of the sequence set forth in SEQ ID NO: 709 (optionally encoding an ASS1 protein comprising the sequence set forth in SEQ ID NO: 230). In another example, the ASS1 coding sequence consists of the sequence set forth in SEQ ID NO: 709 (optionally encoding an ASS1 protein comprising the sequence set forth in SEQ ID NO: 230). Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230 (i.e., protein encoded by exons 3-14 of human ASS1).

In one example, the ASS1 coding sequence is (or comprises a sequence) at least 95%, at least 96%, at least 97%, at least 98%, at least 99%, at least 99.5%, or 100% identical to SEQ ID NO: 713 (optionally encoding an ASS1 protein comprising the sequence set forth in SEQ ID NO: 230). In another example, the ASS1 coding sequence is (or comprises a sequence) at least 96%, at least 97%, at least 98%, at least 99%, at least 99.5%, or 100% identical to SEQ ID NO: 713 (optionally encoding an ASS1 protein comprising the sequence set forth in SEQ ID NO: 230). In another example, the ASS1 coding sequence is (or comprises a sequence) at least 99%, at least 99.5%, or 100% identical to SEQ ID NO: 713 (optionally encoding an ASS1 protein comprising the sequence set forth in SEQ ID NO: 230). In another example, the ASS1 coding sequence comprises the sequence set forth in SEQ ID NO: 713 (optionally encoding an ASS1 protein comprising the sequence set forth in SEQ ID NO: 230). In another example, the ASS1 coding sequence consists essentially of the sequence set forth in SEQ ID NO: 713 (optionally encoding an ASS1 protein comprising the sequence set forth in SEQ ID NO: 230). In another example, the ASS1 coding sequence consists of the sequence set forth in SEQ ID NO: 713 (optionally encoding an ASS1 protein comprising the sequence set forth in SEQ ID NO: 230). Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230 (i.e., protein encoded by exons 3-14 of human ASS1).

When specific ASS1 nucleic acid constructs sequences are disclosed herein, they are meant to encompass the sequence disclosed or the reverse complement of the sequence. For example, if an ASS1 nucleic acid construct disclosed herein consists of the hypothetical sequence 5′-CTGGACCGA-3′, it is also meant to encompass the reverse complement of that sequence (5′-TCGGTCCAG-3′). Likewise, when bidirectional construct elements are disclosed herein in a specific 5′ to 3′ order, they are also meant to encompass the reverse complement of the order of those elements. Likewise, when unidirectional construct elements are disclosed herein in a specific 5′ to 3′ order, they are also meant to encompass the reverse complement of the order of those elements. One reason for this is that, in many embodiments disclosed herein, the ASS1 nucleic acid constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., Ling et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (2) Bidirectional Constructs

The ASS1 nucleic acid constructs disclosed herein can be bidirectional constructs. Such bidirectional constructs can allow for enhanced insertion and expression of encoded ASS1. When used in combination with a nuclease agent (e.g., CRISPR/Cas system, zinc finger nuclease (ZFN) system; transcription activator-like effector nuclease (TALEN) system) as described herein, the bidirectionality of the nucleic acid construct allows the construct to be inserted in either direction (i.e., is not limited to insertion in one direction) within a target genomic locus, allowing the expression of ASS1 when inserted in either orientation, thereby enhancing expression efficiency, as exemplified herein. For example, when used in combination with a nuclease agent (e.g., CRISPR/Cas system, zinc finger nuclease (ZFN) system; transcription activator-like effector nuclease (TALEN) system) as described herein, the bidirectionality of the nucleic acid construct allows the construct to be inserted in either direction (i.e., is not limited to insertion in one direction) within a cleavage site or target insertion site, allowing the expression of ASS1 when inserted in either orientation, thereby enhancing insertion and expression efficiency, as exemplified herein.

A bidirectional construct as disclosed herein can comprise at least two nucleic acid segments, wherein a first segment comprises a first ASS1 coding sequence, and a second segment comprises the reverse complement of a second ASS1 coding sequence, or vice versa. However, other bidirectional constructs disclosed herein can comprise at least two nucleic acid segments, wherein the first segment comprises an ASS1 coding sequence, and the second segment comprises the reverse complement of a coding sequence for another protein, or vice versa. A reverse complement refers to a sequence that is a complement sequence of a reference sequence, wherein the complement sequence is written in the reverse orientation. For example, for a hypothetical sequence 5′-CTGGACCGA-3′, the perfect complement sequence is 3′-GACCTGGCT-5′, and the perfect reverse complement is written 5′-TCGGTCCAG-3′. A reverse complement sequence need not be perfect and may still encode the same polypeptide or a similar polypeptide as the reference sequence. Due to codon usage redundancy, a reverse complement can diverge from a reference sequence that encodes the same polypeptide. The coding sequences can optionally comprise one or more additional sequences, such as sequences encoding amino- or carboxy-terminal amino acid sequences such as a signal sequence, label sequence (e.g., HiBit), or heterologous functional sequence (e.g., nuclear localization sequence (NLS) or self-cleaving peptide) linked to the ASS1 or other protein.

When specific bidirectional construct sequences are disclosed herein, they are meant to encompass the sequence disclosed or the reverse complement of the sequence. For example, if a bidirectional construct disclosed herein consists of the hypothetical sequence 5′-CTGGACCGA-3′, it is also meant to encompass the reverse complement of that sequence (5′-TCGGTCCAG-3′). Likewise, when bidirectional construct elements are disclosed herein in a specific 5′ to 3′ order, they are also meant to encompass the reverse complement of the order of those elements. For example, if a bidirectional construct is disclosed herein that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. One reason for this is that, in many embodiments disclosed herein, the bidirectional constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., Ling et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

When the at least two segments both encode ASS1, the at least two segments can encode the same ASS1 protein or different ASS1 proteins. The different ASS1 proteins can be at least about 90%, at least about 95%, at least about 96%, at least about 97%, at least about 98%, at least about 99%, or at least about 99.5% identical. For example, the first segment can encode a wild type ASS1 protein or fragment thereof, and the second segment can encode a variant ASS1 protein or fragment thereof, or vice versa. Alternatively, the first segment can encode a first variant ASS1 protein, and the second segment can encode a second variant ASS1 protein that is different from the first variant ASS1 protein. Preferably, the two segments encode the same ASS1 protein (i.e., 100% identical).

Even when the two segments encode the same ASS1 protein, the ASS1 coding sequence in the first segment can differ from the ASS1 coding sequence in the second segment. In some bidirectional constructs, the codon usage in the first coding sequence is the same as the codon usage in the second coding sequence. In other bidirectional constructs, the second coding sequence adopts a different codon usage from the codon usage of the first coding sequence in order to reduce hairpin formation. One or both of the coding sequences can be codon-optimized for expression in a host cell. In some bidirectional constructs, only one of the coding sequences is codon-optimized. In some bidirectional constructs, the first coding sequence is codon-optimized. In some bidirectional constructs, the second coding sequence is codon-optimized. In some bidirectional constructs, both coding sequences are codon-optimized. For example, the second ASS1 coding sequence can be codon optimized or may use one or more alternative codons for one or more amino acids of the same ASS1 (i.e., same amino acid sequence) encoded by the ASS1 coding sequence in the first segment. An alternative codon, as used herein, refers to variations in codon usage for a given amino acid, and it may or may not be a preferred or optimized codon (codon optimized) for a given expression system. Preferred codon usage, or codons that are well-tolerated in a given system of expression, are known.

In one example, the second segment comprises a reverse complement of an ASS1 coding sequence that adopts different codon usage from that of the ASS1 coding sequence in the first segment in order to reduce hairpin formation. Such a reverse complement forms base pairs with fewer than all nucleotides of the coding sequence in the first segment, yet it optionally encodes the same polypeptide. In one example, the reverse complement sequence in the second segment is not substantially complementary (e.g., not more than 70% complementary) to the coding sequence in the first segment. In other cases, however, the second segment comprises a reverse complement sequence that is highly complementary (e.g., at least 90% complementary) to the coding sequence in the first segment.

The second segment (the reverse complement of the second ASS1 coding sequence) can have any percentage of complementarity to the first segment (the first ASS1 coding sequence). For example, the second segment sequence can have at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99% complementarity to the first segment. As another example, the second segment sequence can have at least about 30%, at least about 35%, at least about 40%, at least about 45%, at least about 50%, at least about 55%, at least about 60%, at least about 65%, at least about 70%, at least about 75%, at least about 80%, or at least about 85% complementarity to the first segment. As another example, the second segment sequence can have less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, less than about 95%, less than about 97%, or less than about 99% complementarity to the first segment. As another example, the second segment sequence can have less than about 86%, less than about 87%, less than about 88%, less than about 89%, less than about 90%, less than about 95%, less than about 97%, or less than about 99% complementarity to the first segment. The reverse complement of the second coding sequence can be, in some nucleic acid constructs, not substantially complementary (e.g., not more than 70% complementary) to the first coding sequence, not substantially complementary to a fragment of the first coding sequence, highly complementary (e.g., at least 90% complementary) to the first coding sequence, highly complementary to a fragment of the first coding sequence, about 50% to about 80% identical to the reverse complement of the first coding sequence, or about 60% to about 100% identical to the reverse complement of the first coding sequence. In some embodiments, the second segment can have between about 82% and about 90%, between about 83% and about 89%, between about 84% and about 88%, between about 85% and about 87% complementarity to the first segment.

The bidirectional constructs disclosed herein can be modified to include any suitable structural feature as needed for any particular use and/or that confers one or more desired function. For example, the bidirectional nucleic acid constructs disclosed herein need not comprise a homology arm and/or can be, for example, homology-independent donor constructs. Owing in part to the bidirectional function of the nucleic acid constructs, the bidirectional constructs can be inserted into a genomic locus in either direction as described herein to allow for efficient insertion and/or expression of ASS1.

In some cases, the bidirectional nucleic acid construct does not comprise a promoter that drives the expression of ASS1. For example, the expression of ASS1 can be driven by a promoter of the host cell (e.g., the endogenous ASS1 promoter when the transgene is integrated into a host cell's ASS1 locus). In other cases, the bidirectional nucleic acid construct can comprise one or more promoters operably linked to the ASS1 coding sequences. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some bidirectional constructs can comprise a promoter that drives expression of the first ASS1 coding sequence and/or the reverse complement of a promoter that drives expression of the reverse complement of the second ASS1 coding sequence.

The bidirectional constructs disclosed herein can be modified to include or exclude any suitable structural feature as needed for any particular use and/or that confers one or more desired functions. For example, some bidirectional nucleic acid constructs disclosed herein do not comprise a homology arm. Owing in part to the bidirectional function of the nucleic acid construct, the bidirectional construct can be inserted into a genomic locus in either direction (orientation) as described herein to allow for efficient insertion and/or expression of a heterologous ASS1.

The bidirectional constructs can, in some cases, comprise one or more (e.g., two) polyadenylation tail sequences or polyadenylation signal sequences. In some bidirectional constructs, the first segment can comprise a polyadenylation signal sequence. In some bidirectional constructs, the second segment can comprise a polyadenylation signal sequence. In some bidirectional constructs, the first segment can comprise a first polyadenylation signal sequence, and the second segment can comprise a second polyadenylation signal sequence (e.g., a reverse complement of a polyadenylation signal sequence). In some bidirectional constructs, the first segment can comprise a first polyadenylation signal sequence located 3′ of the first coding sequence. In some bidirectional constructs, the second segment can comprise a reverse complement of a second polyadenylation signal sequence located 5′ of the reverse complement of the second coding sequence. In some bidirectional constructs, the first segment can comprise a first polyadenylation signal sequence located 3′ of the first coding sequence, and the second segment can comprise a reverse complement of a second polyadenylation signal sequence located 5′ of the reverse complement of the second coding sequence. The first and second polyadenylation signal sequences can be the same or different. In one example, the first and second polyadenylation signals are different. In a specific example, the first polyadenylation signal is a simian virus 40 (SV40) late polyadenylation signal (or a variant thereof), and the second polyadenylation signal is a bovine growth hormone (BGH) polyadenylation signal (or a variant thereof), or vice versa. In a specific example, one polyadenylation signal can comprise, consist essentially of, or consist of any one of SEQ ID NOS: 239-240, and the other polyadenylation signal can comprise, consist essentially of, or consist of any one of SEQ ID NOS: 236-238. In a specific example, one polyadenylation signal can comprise, consist essentially of, or consist of SEQ ID NO: 239, and the other polyadenylation signal can comprise, consist essentially of, or consist of SEQ ID NO: 237.

In some bidirectional constructs, both the first segment and the second segment comprise a polyadenylation tail sequence. Methods of designing a suitable polyadenylation tail sequence are known. For example, in some bidirectional constructs, one or both of the first and second segment comprises a polyadenylation tail sequence and/or a polyadenylation signal sequence downstream of an open reading frame (i.e., a polyadenylation tail sequence and/or a polyadenylation signal sequence 3′ of a coding sequence, or a reverse complement of a polyadenylation tail sequence and/or a polyadenylation signal sequence 5′ of a reverse complement of a coding sequence). The polyadenylation tail sequence can be encoded, for example, as a “poly-A” stretch downstream of the ASS1 coding sequence (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes. In some bidirectional constructs, a single bidirectional terminator can be used to terminate RNA polymerase transcription in either the sense or the antisense direction (i.e., to terminate RNA polymerase transcription from both the first segment and the second segment). Examples of bidirectional terminators include the ARO4, TRP1, TRP4, ADH1, CYC1, GAL1, GAL7, and GAL10 terminators.

The bidirectional constructs can, in some cases, comprise one or more (e.g., two) splice acceptor sites. In some bidirectional constructs, the first segment can comprise a splice acceptor site. In some bidirectional constructs, the second segment can comprise a splice acceptor site. In some bidirectional constructs, the first segment can comprise a first splice acceptor site, and the second segment can comprise a second splice acceptor site (e.g., a reverse complement of a splice acceptor site). In some bidirectional constructs, the first segment comprises a first splice acceptor site located 5′ of the first coding sequence. In some bidirectional constructs, the second segment comprises a reverse complement of a second splice acceptor site located 3′ of the reverse complement of the second coding sequence. In some bidirectional constructs, the first segment comprises a first splice acceptor site located 5′ of the first coding sequence, and the second segment comprises a reverse complement of a second splice acceptor site located 3′ of the reverse complement of the second coding sequence. The first and second splice acceptor sites can be the same or different. In a specific example, both splice acceptors are human ASS1 exon 3 splice acceptors. In another specific example, both splice acceptors are human ASS1 exon 2 splice acceptors. In another example, both splice acceptors are mouse Ass1 exon 2 splice acceptors. In another example, both splice acceptors are mouse Alb exon 2 splice acceptors. In a specific example, both splice acceptors can comprise, consist essentially of, or consist of SEQ ID NO: 248.

A bidirectional construct may comprise a first coding sequence that encodes a first coding sequence linked to a splice acceptor and a reverse complement of a second coding sequence operably linked to the reverse complement of a splice acceptor. The bidirectional constructs disclosed herein can also comprise a splice acceptor site on either or both ends of the construct, or splice acceptor sites in both the first segment and the second segment (e.g., a splice acceptor site 5′ of a coding sequence, or a reverse complement of a splice acceptor 3′ of a reverse complement of a coding sequence). The splice acceptor site can, for example, comprise NAG or consist of NAG. In a specific example, the splice acceptor is an ASS1 splice acceptor (e.g., an ASS1 splice acceptor used in the splicing together of exons 2 and 3 of ASS1 (i.e., ASS1 exon 3 splice acceptor) or an ASS1 splice acceptor used in the splicing together of exons 1 and 3 of ASS1 (i.e., ASS1 exon 2 splice acceptor). For example, such a splice acceptor can be derived from the human ASS1 gene (e.g., an ASS1 splice acceptor used in the splicing together of exons 2 and 3 of human ASS1 (i.e., human ASS1 exon 3 splice acceptor)). In another example, the splice acceptor can be derived from the mouse Ass1 gene (e.g., an ASS1 splice acceptor used in the splicing together of exons 1 and 2 of mouse Ass1 (i.e., mouse Ass1 exon 2 splice acceptor)). In another example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes. The splice acceptors used in a bidirectional construct may be the same or different. In a specific example, both splice acceptors are human ASS1 exon 3 splice acceptors. In another specific example, both splice acceptors are human ASS1 exon 2 splice acceptors. In another example, both splice acceptors are mouse Ass1 exon 2 splice acceptors. In another example, both splice acceptors are mouse Alb exon 2 splice acceptors. In a specific example, both splice acceptors can comprise, consist essentially of, or consist of SEQ ID NO: 248.

The bidirectional constructs can be circular or linear. For example, a bidirectional construct can be linear. The first and second segments can be joined in a linear manner through a linker sequence. For example, the 5′ end of the second segment that comprises a reverse complement sequence can be linked to the 3′ end of the first segment. Alternatively, the 5′ end of the first segment can be linked to the 3′ end of the second segment that comprises a reverse complement sequence. The linker can be any suitable length. For example, the linker can be between about 5 to about 2000 nucleotides in length. As an example, the linker sequence can be about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 150, about 200, about 250, about 300, about 500, about 1000, about 1500, about 2000, or more nucleotides in length. Other structural elements in addition to, or instead of, a linker sequence, can also be inserted between the first and second segments.

The bidirectional constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the bidirectional construct is single-stranded (e.g., single-stranded DNA).

The bidirectional constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Similarly, one or both ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the bidirectional constructs disclosed herein can be introduced into a cell as part of a vector having additional sequences such as, for example, replication origins, promoters, and genes encoding antibiotic resistance. The constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

The ASS1 coding sequences in the bidirectional constructs disclosed herein may include one or more modifications such as codon optimization (e.g., to human codons), depletion of CpG dinucleotides, mutation of cryptic splice sites, addition of one or more glycosylation sites, or any combination thereof.

In an exemplary bidirectional construct, the second segment is located 3′ of the first segment, the first ASS1 coding sequence and the second ASS1 coding sequence both encode the same human ASS1 protein, the second ASS1 coding sequence adopts a different codon usage from the codon usage of the first ASS1 coding sequence, the first segment comprises a first polyadenylation signal sequence located 3′ of the first ASS1 coding sequence, the second segment comprises a reverse complement of a second polyadenylation signal sequence located 5′ of the reverse complement of the second ASS1 coding sequence, the first segment comprises a first splice acceptor site located 5′ of the first ASS1 coding sequence, the second segment comprises a reverse complement of a second splice acceptor site located 3′ of the reverse complement of the second ASS1 coding sequence, the nucleic acid construct does not comprise a promoter that drives expression of the first ASS1 protein or the second ASS1 protein, and optionally the nucleic acid construct does not comprise a homology arm.

In one example, the one of the ASS1 coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 229. In another example, the one of the ASS1 coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 229 and encodes an ASS1 protein (or an ASS1 protein comprising a sequence) at least 99%, at least 99.5%, or 100% identical to SEQ ID NO: 230. In another example, the one of the ASS1 coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 229 and encodes an ASS1 protein comprising the sequence set forth in SEQ ID NO: 230. In another example, the one of the ASS1 coding sequences is (or comprises a sequence) at least 95%, at least 96%, at least 97%, at least 98%, at least 99%, at least 99.5%, or 100% identical to SEQ ID NO: 229. In another example, the one of the ASS1 coding sequences is (or comprises a sequence) at least 95%, at least 96%, at least 97%, at least 98%, at least 99%, at least 99.5%, or 100% identical to SEQ ID NO: 229 and encodes an ASS1 protein (or an ASS1 protein comprising a sequence) at least 99%, at least 99.5%, or 100% identical to SEQ ID NO: 230. In another example, the one of the ASS1 coding sequences is (or comprises a sequence) at least 95%, at least 96%, at least 97%, at least 98%, at least 99%, at least 99.5%, or 100% identical to SEQ ID NO: 229 and encodes an ASS1 protein comprising the sequence set forth in SEQ ID NO: 230. In another example, the one of the ASS1 coding sequences is (or comprises a sequence) at least 99%, at least 99.5%, or 100% identical to SEQ ID NO: 229. In another example, the one of the ASS1 coding sequences is (or comprises a sequence) at least 99%, at least 99.5%, or 100% identical to SEQ ID NO: 229 and encodes an ASS1 protein (or an ASS1 protein comprising a sequence) at least 99%, at least 99.5%, or 100% identical to SEQ ID NO: 230. In another example, the one of the ASS1 coding sequences is (or comprises a sequence) at least 99%, at least 99.5%, or 100% identical to SEQ ID NO: 229 and encodes an ASS1 protein comprising the sequence set forth in SEQ ID NO: 230. In another example, the one of the ASS1 coding sequences comprises the sequence set forth in SEQ ID NO: 229. In another example, the one of the ASS1 coding sequences consists essentially of the sequence set forth in SEQ ID NO: 229. In another example, the one of the ASS1 coding sequences consists of the sequence set forth in SEQ ID NO: 229. Optionally, the one of the ASS1 coding sequences encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the one of the ASS1 coding sequences in the above examples encodes an ASS1 protein comprising the sequence set forth in SEQ ID NO: 230. Optionally, the one of the ASS1 coding sequences in the above examples encodes an ASS1 protein consisting essentially of the sequence set forth in SEQ ID NO: 230. Optionally, the one of the ASS1 coding sequences in the above examples encodes an ASS1 protein consisting of the sequence set forth in SEQ ID NO: 230.

In one example, the one of the ASS1 coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 228. In another example, the one of the ASS1 coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 228 and encodes an ASS1 protein (or an ASS1 protein comprising a sequence) at least 99%, at least 99.5%, or 100% identical to SEQ ID NO: 230. In another example, the one of the ASS1 coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 228 and encodes an ASS1 protein comprising the sequence set forth in SEQ ID NO: 230. In another example, the one of the ASS1 coding sequences is (or comprises a sequence) at least 95%, at least 96%, at least 97%, at least 98%, at least 99%, at least 99.5%, or 100% identical to SEQ ID NO: 228. In another example, the one of the ASS1 coding sequences is (or comprises a sequence) at least 95%, at least 96%, at least 97%, at least 98%, at least 99%, at least 99.5%, or 100% identical to SEQ ID NO: 228 and encodes an ASS1 protein (or an ASS1 protein comprising a sequence) at least 99%, at least 99.5%, or 100% identical to SEQ ID NO: 230. In another example, the one of the ASS1 coding sequences is (or comprises a sequence) at least 95%, at least 96%, at least 97%, at least 98%, at least 99%, at least 99.5%, or 100% identical to SEQ ID NO: 228 and encodes an ASS1 protein comprising the sequence set forth in SEQ ID NO: 230. In another example, the one of the ASS1 coding sequences is (or comprises a sequence) at least 99%, at least 99.5%, or 100% identical to SEQ ID NO: 228. In another example, the one of the ASS1 coding sequences is (or comprises a sequence) at least 99%, at least 99.5%, or 100% identical to SEQ ID NO: 228 and encodes an ASS1 protein (or an ASS1 protein comprising a sequence) at least 99%, at least 99.5%, or 100% identical to SEQ ID NO: 230. In another example, the one of the ASS1 coding sequences is (or comprises a sequence) at least 99%, at least 99.5%, or 100% identical to SEQ ID NO: 228 and encodes an ASS1 protein comprising the sequence set forth in SEQ ID NO: 230. In another example, the one of the ASS1 coding sequences comprises the sequence set forth in SEQ ID NO: 228. In another example, the one of the ASS1 coding sequences consists essentially of the sequence set forth in SEQ ID NO: 228. In another example, the one of the ASS1 coding sequences consists of the sequence set forth in SEQ ID NO: 228. Optionally, the one of the ASS1 coding sequences encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the one of the ASS1 coding sequences in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the one of the ASS1 coding sequences in the above examples encodes an ASS1 protein consisting of the sequence set forth in SEQ ID NO: 230.

In one example, the one of the ASS1 coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 228. In another example, the one of the ASS1 coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 228 and encodes an ASS1 protein (or an ASS1 protein comprising a sequence) at least 99%, at least 99.5%, or 100% identical to SEQ ID NO: 230. In another example, the one of the ASS1 coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 228 and encodes an ASS1 protein comprising the sequence set forth in SEQ ID NO: 230. In another example, the one of the ASS1 coding sequences is (or comprises a sequence) at least 95%, at least 96%, at least 97%, at least 98%, at least 99%, at least 99.5%, or 100% identical to SEQ ID NO: 228. In another example, the one of the ASS1 coding sequences is (or comprises a sequence) at least 95%, at least 96%, at least 97%, at least 98%, at least 99%, at least 99.5%, or 100% identical to SEQ ID NO: 228 and encodes an ASS1 protein (or an ASS1 protein comprising a sequence) at least 99%, at least 99.5%, or 100% identical to SEQ ID NO: 230. In another example, the one of the ASS1 coding sequences is (or comprises a sequence) at least 95%, at least 96%, at least 97%, at least 98%, at least 99%, at least 99.5%, or 100% identical to SEQ ID NO: 228 and encodes an ASS1 protein comprising the sequence set forth in SEQ ID NO: 230. In another example, the one of the ASS1 coding sequences is (or comprises a sequence) at least 99%, at least 99.5%, or 100% identical to SEQ ID NO: 228. In another example, the one of the ASS1 coding sequences is (or comprises a sequence) at least 99%, at least 99.5%, or 100% identical to SEQ ID NO: 228 and encodes an ASS1 protein (or an ASS1 protein comprising a sequence) at least 99%, at least 99.5%, or 100% identical to SEQ ID NO: 230. In another example, the one of the ASS1 coding sequences is (or comprises a sequence) at least 99%, at least 99.5%, or 100% identical to SEQ ID NO: 228 and encodes an ASS1 protein comprising the sequence set forth in SEQ ID NO: 230. In another example, the one of the ASS1 coding sequences comprises the sequence set forth in SEQ ID NO: 228. In another example, the one of the ASS1 coding sequences consists essentially of the sequence set forth in SEQ ID NO: 228. In another example, the one of the ASS1 coding sequences consists of the sequence set forth in SEQ ID NO: 228. In one example, the other ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 229. In another example, the other ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 229 and encodes an ASS1 protein (or an ASS1 protein comprising a sequence) at least 99%, at least 99.5%, or 100% identical to SEQ ID NO: 230. In another example, the other ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 229 and encodes an ASS1 protein comprising the sequence set forth in SEQ ID NO: 230. In another example, the other ASS1 coding sequence is (or comprises a sequence) at least 95%, at least 96%, at least 97%, at least 98%, at least 99%, at least 99.5%, or 100% identical to SEQ ID NO: 229. In another example, the other ASS1 coding sequence is (or comprises a sequence) at least 95%, at least 96%, at least 97%, at least 98%, at least 99%, at least 99.5%, or 100% identical to SEQ ID NO: 229 and encodes an ASS1 protein (or an ASS1 protein comprising a sequence) at least 99%, at least 99.5%, or 100% identical to SEQ ID NO: 230. In another example, the other ASS1 coding sequence is (or comprises a sequence) at least 95%, at least 96%, at least 97%, at least 98%, at least 99%, at least 99.5%, or 100% identical to SEQ ID NO: 229 and encodes an ASS1 protein comprising the sequence set forth in SEQ ID NO: 230. In another example, the other ASS1 coding sequence is (or comprises a sequence) at least 99%, at least 99.5%, or 100% identical to SEQ ID NO: 229. In another example, the other ASS1 coding sequence is (or comprises a sequence) at least 99%, at least 99.5%, or 100% identical to SEQ ID NO: 229 and encodes an ASS1 protein (or an ASS1 protein comprising a sequence) at least 99%, at least 99.5%, or 100% identical to SEQ ID NO: 230. In another example, the other ASS1 coding sequence is (or comprises a sequence) at least 99%, at least 99.5%, or 100% identical to SEQ ID NO: 229 and encodes an ASS1 protein comprising the sequence set forth in SEQ ID NO: 230. In another example, the other ASS1 coding sequence comprises the sequence set forth in SEQ ID NO: 229. In another example, the other ASS1 coding sequence consists essentially of the sequence set forth in SEQ ID NO: 229. In another example, the other ASS1 coding sequence consists of the sequence set forth in SEQ ID NO: 229. Optionally, one or both of the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, one or both of the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, one or both of the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, one or both of the ASS1 coding sequence in the above examples encodes an ASS1 protein comprising the sequence set forth in SEQ ID NO: 230. Optionally, one or both of the ASS1 coding sequence in the above examples encodes an ASS1 protein consisting essentially of the sequence set forth in SEQ ID NO: 230. Optionally, one or both of the ASS1 coding sequence in the above examples encodes an ASS1 protein consisting of the sequence set forth in SEQ ID NO: 230.

In one example, one of the ASS1 coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 706 and 710 (optionally encoding an ASS1 protein comprising the sequence set forth in SEQ ID NO: 230). In another example, one of the ASS1 coding sequences is (or comprises a sequence) at least 95%, at least 96%, at least 97%, at least 98%, at least 99%, at least 99.5%, or 100% identical to any one of SEQ ID NOS: 706 and 710 (optionally encoding an ASS1 protein comprising the sequence set forth in SEQ ID NO: 230). In another example, one of the ASS1 coding sequences is (or comprises a sequence) at least 99%, at least 99.5%, or 100% identical to any one of SEQ ID NOS: 706 and 710 (optionally encoding an ASS1 protein comprising the sequence set forth in SEQ ID NO: 230). In another example, one of the ASS1 coding sequences comprises the sequence set forth in any one of SEQ ID NOS: 706 and 710 (optionally encoding an ASS1 protein comprising the sequence set forth in SEQ ID NO: 230). In another example, one of the ASS1 coding sequences consists essentially of the sequence set forth in any one of SEQ ID NOS: 706 and 710 (optionally encoding an ASS1 protein comprising the sequence set forth in SEQ ID NO: 230). In another example, one of the ASS1 coding sequences consists of the sequence set forth in any one of SEQ ID NOS: 706 and 710 (optionally encoding an ASS1 protein comprising the sequence set forth in SEQ ID NO: 230). Optionally, the one of the ASS1 coding sequences encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the one of the ASS1 coding sequences in the above examples encodes an ASS1 protein comprising the sequence set forth in SEQ ID NO: 230. Optionally, the one of the ASS1 coding sequences in the above examples encodes an ASS1 protein consisting essentially of the sequence set forth in SEQ ID NO: 230. Optionally, the one of the ASS1 coding sequences in the above examples encodes an ASS1 protein consisting of the sequence set forth in SEQ ID NO: 230 (i.e., protein encoded by exons 3-14 of human ASS1).

In one example, one of the ASS1 coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 706 (optionally encoding an ASS1 protein comprising the sequence set forth in SEQ ID NO: 230). In another example, one of the ASS1 coding sequences is (or comprises a sequence) at least 95%, at least 96%, at least 97%, at least 98%, at least 99%, at least 99.5%, or 100% identical to SEQ ID NO: 706 (optionally encoding an ASS1 protein comprising the sequence set forth in SEQ ID NO: 230). In another example, one of the ASS1 coding sequences is (or comprises a sequence) at least 99%, at least 99.5%, or 100% identical to SEQ ID NO: 706 (optionally encoding an ASS1 protein comprising the sequence set forth in SEQ ID NO: 230). In another example, one of the ASS1 coding sequences comprises the sequence set forth in SEQ ID NO: 706 (optionally encoding an ASS1 protein comprising the sequence set forth in SEQ ID NO: 230). In another example, one of the ASS1 coding sequences consists essentially of the sequence set forth in SEQ ID NO: 706 (optionally encoding an ASS1 protein comprising the sequence set forth in SEQ ID NO: 230). In another example, one of the ASS1 coding sequences consists of the sequence set forth in SEQ ID NO: 706 (optionally encoding an ASS1 protein comprising the sequence set forth in SEQ ID NO: 230). Optionally, the one of the ASS1 coding sequences encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the one of the ASS1 coding sequences in the above examples encodes an ASS1 protein comprising the sequence set forth in SEQ ID NO: 230. Optionally, the one of the ASS1 coding sequences in the above examples encodes an ASS1 protein consisting essentially of the sequence set forth in SEQ ID NO: 230. Optionally, the one of the ASS1 coding sequences in the above examples encodes an ASS1 protein consisting of the sequence set forth in SEQ ID NO: 230 (i.e., protein encoded by exons 3-14 of human ASS1).

In one example, one of the ASS1 coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 710 (optionally encoding an ASS1 protein comprising the sequence set forth in SEQ ID NO: 230). In another example, one of the ASS1 coding sequences is (or comprises a sequence) at least 95%, at least 96%, at least 97%, at least 98%, at least 99%, at least 99.5%, or 100% identical to SEQ ID NO: 710 (optionally encoding an ASS1 protein comprising the sequence set forth in SEQ ID NO: 230). In another example, one of the ASS1 coding sequences is (or comprises a sequence) at least 99%, at least 99.5%, or 100% identical to SEQ ID NO: 710 (optionally encoding an ASS1 protein comprising the sequence set forth in SEQ ID NO: 230). In another example, one of the ASS1 coding sequences comprises the sequence set forth in SEQ ID NO: 710 (optionally encoding an ASS1 protein comprising the sequence set forth in SEQ ID NO: 230). In another example, one of the ASS1 coding sequences consists essentially of the sequence set forth in SEQ ID NO: 710 (optionally encoding an ASS1 protein comprising the sequence set forth in SEQ ID NO: 230). In another example, one of the ASS1 coding sequences consists of the sequence set forth in SEQ ID NO: 710 (optionally encoding an ASS1 protein comprising the sequence set forth in SEQ ID NO: 230). Optionally, the one of the ASS1 coding sequences encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the one of the ASS1 coding sequences in the above examples encodes an ASS1 protein comprising the sequence set forth in SEQ ID NO: 230. Optionally, the one of the ASS1 coding sequences in the above examples encodes an ASS1 protein consisting essentially of the sequence set forth in SEQ ID NO: 230. Optionally, the one of the ASS1 coding sequences in the above examples encodes an ASS1 protein consisting of the sequence set forth in SEQ ID NO: 230 (i.e., protein encoded by exons 3-14 of human ASS1).

In one example, one of the ASS1 coding sequences is (or comprises a sequence) at least 95%, at least 96%, at least 97%, at least 98%, at least 99%, at least 99.5%, or 100% identical to any one of SEQ ID NOS: 705, 709, and 713 (optionally encoding an ASS1 protein comprising the sequence set forth in SEQ ID NO: 230). In another example, one of the ASS1 coding sequences is (or comprises a sequence) at least 96%, at least 97%, at least 98%, at least 99%, at least 99.5%, or 100% identical to any one of SEQ ID NOS: 705, 709, and 713 (optionally encoding an ASS1 protein comprising the sequence set forth in SEQ ID NO: 230). In another example, one of the ASS1 coding sequences is (or comprises a sequence) at least 99%, at least 99.5%, or 100% identical to any one of SEQ ID NOS: 705, 709, and 713 (optionally encoding an ASS1 protein comprising the sequence set forth in SEQ ID NO: 230). In another example, one of the ASS1 coding sequences comprises the sequence set forth in any one of SEQ ID NOS: 705, 709, and 713 (optionally encoding an ASS1 protein comprising the sequence set forth in SEQ ID NO: 230). In another example, one of the ASS1 coding sequences consists essentially of the sequence set forth in any one of SEQ ID NOS: 705, 709, and 713 (optionally encoding an ASS1 protein comprising the sequence set forth in SEQ ID NO: 230). In another example, one of the ASS1 coding sequences consists of the sequence set forth in any one of SEQ ID NOS: 705, 709, and 713 (optionally encoding an ASS1 protein comprising the sequence set forth in SEQ ID NO: 230). Optionally, the one of the ASS1 coding sequences encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the one of the ASS1 coding sequences in the above examples encodes an ASS1 protein comprising the sequence set forth in SEQ ID NO: 230. Optionally, the one of the ASS1 coding sequences in the above examples encodes an ASS1 protein consisting essentially of the sequence set forth in SEQ ID NO: 230. Optionally, the one of the ASS1 coding sequences in the above examples encodes an ASS1 protein consisting of the sequence set forth in SEQ ID NO: 230 (i.e., protein encoded by exons 3-14 of human ASS1).

In one example, one of the ASS1 coding sequences is (or comprises a sequence) at least 95%, at least 96%, at least 97%, at least 98%, at least 99%, at least 99.5%, or 100% identical to SEQ ID NO: 705 (optionally encoding an ASS1 protein comprising the sequence set forth in SEQ ID NO: 230). In another example, one of the ASS1 coding sequences is (or comprises a sequence) at least 96%, at least 97%, at least 98%, at least 99%, at least 99.5%, or 100% identical to SEQ ID NO: 705 (optionally encoding an ASS1 protein comprising the sequence set forth in SEQ ID NO: 230). In another example, one of the ASS1 coding sequences is (or comprises a sequence) at least 99%, at least 99.5%, or 100% identical to SEQ ID NO: 705 (optionally encoding an ASS1 protein comprising the sequence set forth in SEQ ID NO: 230). In another example, one of the ASS1 coding sequences comprises the sequence set forth in SEQ ID NO: 705 (optionally encoding an ASS1 protein comprising the sequence set forth in SEQ ID NO: 230). In another example, one of the ASS1 coding sequences consists essentially of the sequence set forth in SEQ ID NO: 705 (optionally encoding an ASS1 protein comprising the sequence set forth in SEQ ID NO: 230). In another example, one of the ASS1 coding sequences consists of the sequence set forth in SEQ ID NO: 705 (optionally encoding an ASS1 protein comprising the sequence set forth in SEQ ID NO: 230). Optionally, the one of the ASS1 coding sequences encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the one of the ASS1 coding sequences in the above examples encodes an ASS1 protein comprising the sequence set forth in SEQ ID NO: 230. Optionally, the one of the ASS1 coding sequences in the above examples encodes an ASS1 protein consisting essentially of the sequence set forth in SEQ ID NO: 230. Optionally, the one of the ASS1 coding sequences in the above examples encodes an ASS1 protein consisting of the sequence set forth in SEQ ID NO: 230 (i.e., protein encoded by exons 3-14 of human ASS1).

In one example, one of the ASS1 coding sequences is (or comprises a sequence) at least 95%, at least 96%, at least 97%, at least 98%, at least 99%, at least 99.5%, or 100% identical to SEQ ID NO: 709 (optionally encoding an ASS1 protein comprising the sequence set forth in SEQ ID NO: 230). In another example, one of the ASS1 coding sequences is (or comprises a sequence) at least 96%, at least 97%, at least 98%, at least 99%, at least 99.5%, or 100% identical to SEQ ID NO: 709 (optionally encoding an ASS1 protein comprising the sequence set forth in SEQ ID NO: 230). In another example, one of the ASS1 coding sequences is (or comprises a sequence) at least 99%, at least 99.5%, or 100% identical to SEQ ID NO: 709 (optionally encoding an ASS1 protein comprising the sequence set forth in SEQ ID NO: 230). In another example, one of the ASS1 coding sequences comprises the sequence set forth in SEQ ID NO: 709 (optionally encoding an ASS1 protein comprising the sequence set forth in SEQ ID NO: 230). In another example, one of the ASS1 coding sequences consists essentially of the sequence set forth in SEQ ID NO: 709 (optionally encoding an ASS1 protein comprising the sequence set forth in SEQ ID NO: 230). In another example, one of the ASS1 coding sequences consists of the sequence set forth in SEQ ID NO: 709 (optionally encoding an ASS1 protein comprising the sequence set forth in SEQ ID NO: 230). Optionally, the one of the ASS1 coding sequences encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the one of the ASS1 coding sequences in the above examples encodes an ASS1 protein comprising the sequence set forth in SEQ ID NO: 230. Optionally, the one of the ASS1 coding sequences in the above examples encodes an ASS1 protein consisting essentially of the sequence set forth in SEQ ID NO: 230. Optionally, the one of the ASS1 coding sequences in the above examples encodes an ASS1 protein consisting of the sequence set forth in SEQ ID NO: 230 (i.e., protein encoded by exons 3-14 of human ASS1).

In one example, one of the ASS1 coding sequences is (or comprises a sequence) at least 95%, at least 96%, at least 97%, at least 98%, at least 99%, at least 99.5%, or 100% identical to SEQ ID NO: 713 (optionally encoding an ASS1 protein comprising the sequence set forth in SEQ ID NO: 230). In another example, one of the ASS1 coding sequences is (or comprises a sequence) at least 96%, at least 97%, at least 98%, at least 99%, at least 99.5%, or 100% identical to SEQ ID NO: 713 (optionally encoding an ASS1 protein comprising the sequence set forth in SEQ ID NO: 230). In another example, one of the ASS1 coding sequences is (or comprises a sequence) at least 99%, at least 99.5%, or 100% identical to SEQ ID NO: 713 (optionally encoding an ASS1 protein comprising the sequence set forth in SEQ ID NO: 230). In another example, one of the ASS1 coding sequences comprises the sequence set forth in SEQ ID NO: 713 (optionally encoding an ASS1 protein comprising the sequence set forth in SEQ ID NO: 230). In another example, one of the ASS1 coding sequences consists essentially of the sequence set forth in SEQ ID NO: 713 (optionally encoding an ASS1 protein comprising the sequence set forth in SEQ ID NO: 230). In another example, one of the ASS1 coding sequences consists of the sequence set forth in SEQ ID NO: 713 (optionally encoding an ASS1 protein comprising the sequence set forth in SEQ ID NO: 230). Optionally, the one of the ASS1 coding sequences encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the one of the ASS1 coding sequences in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the one of the ASS1 coding sequences in the above examples encodes an ASS1 protein comprising the sequence set forth in SEQ ID NO: 230. Optionally, the one of the ASS1 coding sequences in the above examples encodes an ASS1 protein consisting essentially of the sequence set forth in SEQ ID NO: 230. Optionally, the one of the ASS1 coding sequences in the above examples encodes an ASS1 protein consisting of the sequence set forth in SEQ ID NO: 230 (i.e., protein encoded by exons 3-14 of human ASS1).

In one example, the first ASS1 coding sequence is (or comprises a sequence) at least 95%, at least 96%, at least 97%, at least 98%, at least 99%, at least 99.5%, or 100% identical to SEQ ID NO: 705 (optionally encoding an ASS1 protein comprising the sequence set forth in SEQ ID NO: 230). In another example, the first ASS1 coding sequence is (or comprises a sequence) at least 96%, at least 97%, at least 98%, at least 99%, at least 99.5%, or 100% identical to SEQ ID NO: 705 (optionally encoding an ASS1 protein comprising the sequence set forth in SEQ ID NO: 230). In another example, the first ASS1 coding sequence is (or comprises a sequence) at least 99%, at least 99.5%, or 100% identical to SEQ ID NO: 705 (optionally encoding an ASS1 protein comprising the sequence set forth in SEQ ID NO: 230). In another example, the first ASS1 coding sequence comprises the sequence set forth in SEQ ID NO: 705 (optionally encoding an ASS1 protein comprising the sequence set forth in SEQ ID NO: 230). In another example, the first ASS1 coding sequence consists essentially of the sequence set forth in SEQ ID NO: 705 (optionally encoding an ASS1 protein comprising the sequence set forth in SEQ ID NO: 230). In another example, the first ASS1 coding sequence consists of the sequence set forth in SEQ ID NO: 705 (optionally encoding an ASS1 protein comprising the sequence set forth in SEQ ID NO: 230). Optionally, the first ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the first ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the first ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the first ASS1 coding sequence in the above examples encodes an ASS1 protein comprising the sequence set forth in SEQ ID NO: 230. Optionally, the first ASS1 coding sequence in the above examples encodes an ASS1 protein consisting essentially of the sequence set forth in SEQ ID NO: 230. Optionally, the first ASS1 coding sequence in the above examples encodes an ASS1 protein consisting of the sequence set forth in SEQ ID NO: 230 (i.e., protein encoded by exons 3-14 of human ASS1). In one example, the second ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 706 (optionally encoding an ASS1 protein comprising the sequence set forth in SEQ ID NO: 230). In another example, the second ASS1 coding sequence is (or comprises a sequence) at least 95%, at least 96%, at least 97%, at least 98%, at least 99%, at least 99.5%, or 100% identical to any one of SEQ ID NOS: 706 (optionally encoding an ASS1 protein comprising the sequence set forth in SEQ ID NO: 230). In another example, the second ASS1 coding sequence is (or comprises a sequence) at least 99%, at least 99.5%, or 100% identical to any one of SEQ ID NOS: 706 (optionally encoding an ASS1 protein comprising the sequence set forth in SEQ ID NO: 230). In another example, the second ASS1 coding sequence comprises the sequence set forth in any one of SEQ ID NOS: 706 (optionally encoding an ASS1 protein comprising the sequence set forth in SEQ ID NO: 230). In another example, the second ASS1 coding sequence consists essentially of the sequence set forth in any one of SEQ ID NOS: 706 (optionally encoding an ASS1 protein comprising the sequence set forth in SEQ ID NO: 230). In another example, the second ASS1 coding sequence consists of the sequence set forth in any one of SEQ ID NOS: 706 (optionally encoding an ASS1 protein comprising the sequence set forth in SEQ ID NO: 230). Optionally, the second ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the second ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the second ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the second ASS1 coding sequence in the above examples encodes an ASS1 protein comprising the sequence set forth in SEQ ID NO: 230. Optionally, the second ASS1 coding sequence in the above examples encodes an ASS1 protein consisting essentially of the sequence set forth in SEQ ID NO: 230. Optionally, the second ASS1 coding sequence in the above examples encodes an ASS1 protein consisting of the sequence set forth in SEQ ID NO: 230 (i.e., protein encoded by exons 3-14 of human ASS1).

In one example, the first ASS1 coding sequence is (or comprises a sequence) at least 95%, at least 96%, at least 97%, at least 98%, at least 99%, at least 99.5%, or 100% identical to SEQ ID NO: 709 (optionally encoding an ASS1 protein comprising the sequence set forth in SEQ ID NO: 230). In another example, the first ASS1 coding sequence is (or comprises a sequence) at least 96%, at least 97%, at least 98%, at least 99%, at least 99.5%, or 100% identical to SEQ ID NO: 709 (optionally encoding an ASS1 protein comprising the sequence set forth in SEQ ID NO: 230). In another example, the first ASS1 coding sequence is (or comprises a sequence) at least 99%, at least 99.5%, or 100% identical to SEQ ID NO: 709 (optionally encoding an ASS1 protein comprising the sequence set forth in SEQ ID NO: 230). In another example, the first ASS1 coding sequence comprises the sequence set forth in SEQ ID NO: 709 (optionally encoding an ASS1 protein comprising the sequence set forth in SEQ ID NO: 230). In another example, the first ASS1 coding sequence consists essentially of the sequence set forth in SEQ ID NO: 709 (optionally encoding an ASS1 protein comprising the sequence set forth in SEQ ID NO: 230). In another example, the first ASS1 coding sequence consists of the sequence set forth in SEQ ID NO: 709 (optionally encoding an ASS1 protein comprising the sequence set forth in SEQ ID NO: 230). Optionally, the first ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the first ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the first ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the first ASS1 coding sequence in the above examples encodes an ASS1 protein comprising the sequence set forth in SEQ ID NO: 230. Optionally, the first ASS1 coding sequence in the above examples encodes an ASS1 protein consisting essentially of the sequence set forth in SEQ ID NO: 230. Optionally, the first ASS1 coding sequence in the above examples encodes an ASS1 protein consisting of the sequence set forth in SEQ ID NO: 230 (i.e., protein encoded by exons 3-14 of human ASS1). In one example, the second ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 710 (optionally encoding an ASS1 protein comprising the sequence set forth in SEQ ID NO: 230). In another example, the second ASS1 coding sequence is (or comprises a sequence) at least 95%, at least 96%, at least 97%, at least 98%, at least 99%, at least 99.5%, or 100% identical to any one of SEQ ID NOS: 710 (optionally encoding an ASS1 protein comprising the sequence set forth in SEQ ID NO: 230). In another example, the second ASS1 coding sequence is (or comprises a sequence) at least 99%, at least 99.5%, or 100% identical to any one of SEQ ID NOS: 710 (optionally encoding an ASS1 protein comprising the sequence set forth in SEQ ID NO: 230). In another example, the second ASS1 coding sequence comprises the sequence set forth in any one of SEQ ID NOS: 710 (optionally encoding an ASS1 protein comprising the sequence set forth in SEQ ID NO: 230). In another example, the second ASS1 coding sequence consists essentially of the sequence set forth in any one of SEQ ID NOS: 710 (optionally encoding an ASS1 protein comprising the sequence set forth in SEQ ID NO: 230). In another example, the second ASS1 coding sequence consists of the sequence set forth in any one of SEQ ID NOS: 710 (optionally encoding an ASS1 protein comprising the sequence set forth in SEQ ID NO: 230). Optionally, the second ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the second ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the second ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the second ASS1 coding sequence in the above examples encodes an ASS1 protein comprising the sequence set forth in SEQ ID NO: 230. Optionally, the second ASS1 coding sequence in the above examples encodes an ASS1 protein consisting essentially of the sequence set forth in SEQ ID NO: 230. Optionally, the second ASS1 coding sequence in the above examples encodes an ASS1 protein consisting of the sequence set forth in SEQ ID NO: 230 (i.e., protein encoded by exons 3-14 of human ASS1).

In one example, the first ASS1 coding sequence is (or comprises a sequence) at least 95%, at least 96%, at least 97%, at least 98%, at least 99%, at least 99.5%, or 100% identical to SEQ ID NO: 713 (optionally encoding an ASS1 protein comprising the sequence set forth in SEQ ID NO: 230). In another example, the first ASS1 coding sequence is (or comprises a sequence) at least 96%, at least 97%, at least 98%, at least 99%, at least 99.5%, or 100% identical to SEQ ID NO: 713 (optionally encoding an ASS1 protein comprising the sequence set forth in SEQ ID NO: 230). In another example, the first ASS1 coding sequence is (or comprises a sequence) at least 99%, at least 99.5%, or 100% identical to SEQ ID NO: 713 (optionally encoding an ASS1 protein comprising the sequence set forth in SEQ ID NO: 230). In another example, the first ASS1 coding sequence comprises the sequence set forth in SEQ ID NO: 713 (optionally encoding an ASS1 protein comprising the sequence set forth in SEQ ID NO: 230). In another example, the first ASS1 coding sequence consists essentially of the sequence set forth in SEQ ID NO: 713 (optionally encoding an ASS1 protein comprising the sequence set forth in SEQ ID NO: 230). In another example, the first ASS1 coding sequence consists of the sequence set forth in SEQ ID NO: 713 (optionally encoding an ASS1 protein comprising the sequence set forth in SEQ ID NO: 230). Optionally, the first ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the first ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the first ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the first ASS1 coding sequence in the above examples encodes an ASS1 protein comprising the sequence set forth in SEQ ID NO: 230. Optionally, the first ASS1 coding sequence in the above examples encodes an ASS1 protein consisting essentially of the sequence set forth in SEQ ID NO: 230. Optionally, the first ASS1 coding sequence in the above examples encodes an ASS1 protein consisting of the sequence set forth in SEQ ID NO: 230 (i.e., protein encoded by exons 3-14 of human ASS1). In one example, the second ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 706 (optionally encoding an ASS1 protein comprising the sequence set forth in SEQ ID NO: 230). In another example, the second ASS1 coding sequence is (or comprises a sequence) at least 95%, at least 96%, at least 97%, at least 98%, at least 99%, at least 99.5%, or 100% identical to any one of SEQ ID NOS: 706 (optionally encoding an ASS1 protein comprising the sequence set forth in SEQ ID NO: 230). In another example, the second ASS1 coding sequence is (or comprises a sequence) at least 99%, at least 99.5%, or 100% identical to any one of SEQ ID NOS: 706 (optionally encoding an ASS1 protein comprising the sequence set forth in SEQ ID NO: 230). In another example, the second ASS1 coding sequence comprises the sequence set forth in any one of SEQ ID NOS: 706 (optionally encoding an ASS1 protein comprising the sequence set forth in SEQ ID NO: 230). In another example, the second ASS1 coding sequence consists essentially of the sequence set forth in any one of SEQ ID NOS: 706 (optionally encoding an ASS1 protein comprising the sequence set forth in SEQ ID NO: 230). In another example, the second ASS1 coding sequence consists of the sequence set forth in any one of SEQ ID NOS: 706 (optionally encoding an ASS1 protein comprising the sequence set forth in SEQ ID NO: 230). Optionally, the second ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the second ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the second ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the second ASS1 coding sequence in the above examples encodes an ASS1 protein comprising the sequence set forth in SEQ ID NO: 230. Optionally, the second ASS1 coding sequence in the above examples encodes an ASS1 protein consisting essentially of the sequence set forth in SEQ ID NO: 230. Optionally, the second ASS1 coding sequence in the above examples encodes an ASS1 protein consisting of the sequence set forth in SEQ ID NO: 230 (i.e., protein encoded by exons 3-14 of human ASS1).

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to any one of SEQ ID NOS: 703, 704, 707, 708, 711, and 712. In another particular example, an exemplary bidirectional construct comprises a sequence at least 95%, at least 96%, at least 97%, at least 98%, at least 99%, at least 99.5%, or 100% identical to any one of SEQ ID NOS: 703, 704, 707, 708, 711, and 712. In another particular example, an exemplary bidirectional construct comprises a sequence at least 99%, at least 99.5%, or 100% identical to any one of SEQ ID NOS: 703, 704, 707, 708, 711, and 712. In another particular example, an exemplary bidirectional construct comprises any one of SEQ ID NOS: 703, 704, 707, 708, 711, and 712. In another particular example, an exemplary bidirectional construct consists essentially of any one of SEQ ID NOS: 703, 704, 707, 708, 711, and 712. In another particular example, an exemplary bidirectional construct consists of any one of SEQ ID NOS: 703, 704, 707, 708, 711, and 712.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to any one of SEQ ID NOS: 703 and 704. In another particular example, an exemplary bidirectional construct comprises a sequence at least 95%, at least 96%, at least 97%, at least 98%, at least 99%, at least 99.5%, or 100% identical to any one of SEQ ID NOS: 703 and 704. In another particular example, an exemplary bidirectional construct comprises a sequence at least 99%, at least 99.5%, or 100% identical to any one of SEQ ID NOS: 703 and 704. In another particular example, an exemplary bidirectional construct comprises any one of SEQ ID NOS: 703 and 704. In another particular example, an exemplary bidirectional construct consists essentially of any one of SEQ ID NOS: 703 and 704. In another particular example, an exemplary bidirectional construct consists of any one of SEQ ID NOS: 703 and 704.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO 703. In another particular example, an exemplary bidirectional construct comprises a sequence at least 95%, at least 96%, at least 97%, at least 98%, at least 99%, at least 99.5%, or 100% identical to SEQ ID NO 703. In another particular example, an exemplary bidirectional construct comprises a sequence at least 99%, at least 99.5%, or 100% identical to SEQ ID NO 703. In another particular example, an exemplary bidirectional construct comprises SEQ ID NO 703. In another particular example, an exemplary bidirectional construct consists essentially of SEQ ID NO 703. In another particular example, an exemplary bidirectional construct consists of SEQ ID NO 703.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 704. In another particular example, an exemplary bidirectional construct comprises a sequence at least 95%, at least 96%, at least 97%, at least 98%, at least 99%, at least 99.5%, or 100% identical to SEQ ID NO: 704. In another particular example, an exemplary bidirectional construct comprises a sequence at least 99%, at least 99.5%, or 100% identical to SEQ ID NO: 704. In another particular example, an exemplary bidirectional construct comprises SEQ ID NO: 704. In another particular example, an exemplary bidirectional construct consists essentially of SEQ ID NO: 704. In another particular example, an exemplary bidirectional construct consists of SEQ ID NO: 704.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to any one of SEQ ID NOS: 707 and 708. In another particular example, an exemplary bidirectional construct comprises a sequence at least 95%, at least 96%, at least 97%, at least 98%, at least 99%, at least 99.5%, or 100% identical to any one of SEQ ID NOS: 707 and 708. In another particular example, an exemplary bidirectional construct comprises a sequence at least 99%, at least 99.5%, or 100% identical to any one of SEQ ID NOS: 707 and 708. In another particular example, an exemplary bidirectional construct comprises any one of SEQ ID NOS: 707 and 708. In another particular example, an exemplary bidirectional construct consists essentially of any one of SEQ ID NOS: 707 and 708. In another particular example, an exemplary bidirectional construct consists of any one of SEQ ID NOS: 707 and 708.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 707. In another particular example, an exemplary bidirectional construct comprises a sequence at least 95%, at least 96%, at least 97%, at least 98%, at least 99%, at least 99.5%, or 100% identical to SEQ ID NO: 707. In another particular example, an exemplary bidirectional construct comprises a sequence at least 99%, at least 99.5%, or 100% identical to SEQ ID NO: 707. In another particular example, an exemplary bidirectional construct comprises SEQ ID NO: 707. In another particular example, an exemplary bidirectional construct consists essentially of SEQ ID NO: 707. In another particular example, an exemplary bidirectional construct consists of SEQ ID NO: 707.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 708. In another particular example, an exemplary bidirectional construct comprises a sequence at least 95%, at least 96%, at least 97%, at least 98%, at least 99%, at least 99.5%, or 100% identical to SEQ ID NO: 708. In another particular example, an exemplary bidirectional construct comprises a sequence at least 99%, at least 99.5%, or 100% identical to SEQ ID NO: 708. In another particular example, an exemplary bidirectional construct comprises SEQ ID NO: 708. In another particular example, an exemplary bidirectional construct consists essentially of SEQ ID NO: 708. In another particular example, an exemplary bidirectional construct consists of SEQ ID NO: 708.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to any one of SEQ ID NOS: 711 and 712. In another particular example, an exemplary bidirectional construct comprises a sequence at least 95%, at least 96%, at least 97%, at least 98%, at least 99%, at least 99.5%, or 100% identical to any one of SEQ ID NOS: 711 and 712. In another particular example, an exemplary bidirectional construct comprises a sequence at least 99%, at least 99.5%, or 100% identical to any one of SEQ ID NOS: 711 and 712. In another particular example, an exemplary bidirectional construct comprises any one of SEQ ID NOS: 711 and 712. In another particular example, an exemplary bidirectional construct consists essentially of any one of SEQ ID NOS: 711 and 712. In another particular example, an exemplary bidirectional construct consists of any one of SEQ ID NOS: 711 and 712.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 711. In another particular example, an exemplary bidirectional construct comprises a sequence at least 95%, at least 96%, at least 97%, at least 98%, at least 99%, at least 99.5%, or 100% identical to SEQ ID NO: 711. In another particular example, an exemplary bidirectional construct comprises a sequence at least 99%, at least 99.5%, or 100% identical to SEQ ID NO: 711. In another particular example, an exemplary bidirectional construct comprises SEQ ID NO: 711. In another particular example, an exemplary bidirectional construct consists essentially of SEQ ID NO: 711. In another particular example, an exemplary bidirectional construct consists of SEQ ID NO: 711.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 712. In another particular example, an exemplary bidirectional construct comprises a sequence at least 95%, at least 96%, at least 97%, at least 98%, at least 99%, at least 99.5%, or 100% identical to SEQ ID NO: 712. In another particular example, an exemplary bidirectional construct comprises a sequence at least 99%, at least 99.5%, or 100% identical to SEQ ID NO: 712. In another particular example, an exemplary bidirectional construct comprises SEQ ID NO: 712. In another particular example, an exemplary bidirectional construct consists essentially of SEQ ID NO: 712. In another particular example, an exemplary bidirectional construct consists of SEQ ID NO: 712.

### (3) Unidirectional Constructs

The ASS1 nucleic acid constructs disclosed herein can be unidirectional constructs.

When specific unidirectional construct sequences are disclosed herein, they are meant to encompass the sequence disclosed or the reverse complement of the sequence. For example, if a unidirectional construct disclosed herein consists of the hypothetical sequence 5′-CTGGACCGA-3′, it is also meant to encompass the reverse complement of that sequence (5′-TCGGTCCAG-3′). Likewise, when unidirectional construct elements are disclosed herein in a specific 5′ to 3′ order, they are also meant to encompass the reverse complement of the order of those elements. One reason for this is that, in many embodiments disclosed herein, the unidirectional constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., Ling et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

In the unidirectional constructs, the ASS1 coding sequence can be a wild type ASS1 coding sequence without further modification. In the unidirectional constructs, the ASS1 coding sequence can be codon-optimized for expression in a host cell. For example, the ASS1 coding sequence can be codon optimized or may use one or more alternative codons for one or more amino acids of the ASS1 (i.e., same amino acid sequence). An alternative codon, as used herein, refers to variations in codon usage for a given amino acid, and it may or may not be a preferred or optimized codon (codon optimized) for a given expression system. Preferred codon usage, or codons that are well-tolerated in a given system of expression, are known.

The unidirectional constructs disclosed herein can be modified to include any suitable structural feature as needed for any particular use and/or that confers one or more desired functions. For example, the unidirectional nucleic acid constructs disclosed herein need not comprise a homology arm and/or can be, for example, homology-independent donor constructs.

In some cases, the unidirectional nucleic acid construct does not comprise a promoter that drives the expression of ASS1. For example, the expression of ASS1 can be driven by a promoter of the host cell (e.g., the endogenous ASS1 promoter when the transgene is integrated into a host cell's ASS1 locus). In other cases, the unidirectional nucleic acid construct can comprise one or more promoters operably linked to the ASS1 coding sequence. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some unidirectional constructs can comprise a promoter that drives expression of the ASS1 coding sequence.

The unidirectional constructs can, in some cases, comprise one or more polyadenylation tail sequences or polyadenylation signal sequences. Some unidirectional constructs can comprise a polyadenylation signal sequence located 3′ of the ASS1 coding sequence. In one example, the polyadenylation signal is a simian virus 40 (SV40) late polyadenylation signal. For example, the polyadenylation signal can comprise, consist essentially of, or consist of SEQ ID NO: 239 or SEQ ID NO: 240. In another example, the polyadenylation signal is a bovine growth hormone (BGH) polyadenylation signal. For example, the polyadenylation signal can comprise, consist essentially of, or consist of SEQ ID NO: 236, SEQ ID NO: 237, or SEQ ID NO: 238. In another example, the polyadenylation signal is a human growth hormone (HGH) polyadenylation signal. For example, the polyadenylation signal can comprise, consist essentially of, or consist of SEQ ID NO: 235.

Methods of designing a suitable polyadenylation tail sequence are known. For example, some unidirectional constructs comprise a polyadenylation tail sequence and/or a polyadenylation signal sequence downstream of an open reading frame (i.e., a polyadenylation tail sequence and/or a polyadenylation signal sequence 3′ of a coding sequence). The polyadenylation tail sequence can be encoded, for example, as a “poly-A” stretch downstream of the ASS1 coding sequence (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes.

The unidirectional constructs can, in some cases, comprise one or more splice acceptor sites. Some unidirectional constructs comprise a splice acceptor site located 5′ of the ASS1 coding sequence. In a specific example, the splice acceptor is a human ASS1 exon 3 splice acceptor. In another specific example, the splice acceptor is a human ASS1 exon 2 splice acceptor. In another example, the splice acceptor is a mouse Ass1 exon 2 splice acceptor. In another specific example, the splice acceptor is a mouse Alb exon 2 splice acceptor. In a specific example, the splice acceptor can comprise, consist essentially of, or consist of SEQ ID NO: 248.

The splice acceptor site can, for example, comprise NAG or consist of NAG. In a specific example, the splice acceptor is an ASS1 splice acceptor (e.g., an ASS1 splice acceptor used in the splicing together of exons 2 and 3 of ASS1 (i.e., ASS1 exon 3 splice acceptor) or an ASS1 splice acceptor used in the splicing together of exons 1 and 3 of ASS1 (i.e., ASS1 exon 2 splice acceptor). For example, such a splice acceptor can be derived from the human ASS1 gene (e.g., an ASS1 splice acceptor used in the splicing together of exons 2 and 3 of human ASS1 (i.e., human ASS1 exon 3 splice acceptor)). In another example, the splice acceptor can be derived from the mouse Ass1 gene (e.g., an ASS1 splice acceptor used in the splicing together of exons 1 and 2 of mouse Ass1 (i.e., mouse Ass1 exon 2 splice acceptor)). In another example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes.

The unidirectional constructs can be circular or linear. For example, a unidirectional construct can be linear.

The unidirectional constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the unidirectional construct is single-stranded (e.g., single-stranded DNA).

The unidirectional constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Similarly, one or both ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the unidirectional constructs disclosed herein can be introduced into a cell as part of a vector having additional sequences such as, for example, replication origins, promoters, and genes encoding antibiotic resistance. The constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

The ASS1 coding sequences in the unidirectional constructs disclosed herein may include one or more modifications such as codon optimization (e.g., to human codons), depletion of CpG dinucleotides, mutation of cryptic splice sites, addition of one or more glycosylation sites, or any combination thereof.

In an exemplary unidirectional construct, the construct comprises a polyadenylation signal sequence located 3′ of the ASS1 coding sequence, the construct comprises a splice acceptor site located 5′ of the ASS1 coding sequence, and the nucleic acid construct does not comprise a promoter that drives expression of the ASS1 protein, and optionally the nucleic acid construct does not comprise a homology arm.

In one example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 229. In another example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 229 and encodes an ASS1 protein (or an ASS1 protein comprising a sequence) at least 99%, at least 99.5%, or 100% identical to SEQ ID NO: 230. In another example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 229 and encodes an ASS1 protein comprising the sequence set forth in SEQ ID NO: 230. In another example, the ASS1 coding sequence is (or comprises a sequence) at least 95%, at least 96%, at least 97%, at least 98%, at least 99%, at least 99.5%, or 100% identical to SEQ ID NO: 229. In another example, the ASS1 coding sequence is (or comprises a sequence) at least 95%, at least 96%, at least 97%, at least 98%, at least 99%, at least 99.5%, or 100% identical to SEQ ID NO: 229 and encodes an ASS1 protein (or an ASS1 protein comprising a sequence) at least 99%, at least 99.5%, or 100% identical to SEQ ID NO: 230. In another example, the ASS1 coding sequence is (or comprises a sequence) at least 95%, at least 96%, at least 97%, at least 98%, at least 99%, at least 99.5%, or 100% identical to SEQ ID NO: 229 and encodes an ASS1 protein comprising the sequence set forth in SEQ ID NO: 230. In another example, the ASS1 coding sequence is (or comprises a sequence) at least 99%, at least 99.5%, or 100% identical to SEQ ID NO: 229. In another example, the ASS1 coding sequence is (or comprises a sequence) at least 99%, at least 99.5%, or 100% identical to SEQ ID NO: 229 and encodes an ASS1 protein (or an ASS1 protein comprising a sequence) at least 99%, at least 99.5%, or 100% identical to SEQ ID NO: 230. In another example, the ASS1 coding sequence is (or comprises a sequence) at least 99%, at least 99.5%, or 100% identical to SEQ ID NO: 229 and encodes an ASS1 protein comprising the sequence set forth in SEQ ID NO: 230. In another example, the ASS1 coding sequence comprises the sequence set forth in SEQ ID NO: 229. In another example, the ASS1 coding sequence consists essentially of the sequence set forth in SEQ ID NO: 229. In another example, the one of the ASS1 coding sequences consists of the sequence set forth in SEQ ID NO: 229. Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230.

In one example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 228. In another example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 228 and encodes an ASS1 protein (or an ASS1 protein comprising a sequence) at least 99%, at least 99.5%, or 100% identical to SEQ ID NO: 230. In another example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 228 and encodes an ASS1 protein comprising the sequence set forth in SEQ ID NO: 230. In another example, the ASS1 coding sequence is (or comprises a sequence) at least 95%, at least 96%, at least 97%, at least 98%, at least 99%, at least 99.5%, or 100% identical to SEQ ID NO: 228. In another example, the ASS1 coding sequence is (or comprises a sequence) at least 95%, at least 96%, at least 97%, at least 98%, at least 99%, at least 99.5%, or 100% identical to SEQ ID NO: 228 and encodes an ASS1 protein (or an ASS1 protein comprising a sequence) at least 99%, at least 99.5%, or 100% identical to SEQ ID NO: 230. In another example, the ASS1 coding sequence is (or comprises a sequence) at least 95%, at least 96%, at least 97%, at least 98%, at least 99%, at least 99.5%, or 100% identical to SEQ ID NO: 228 and encodes an ASS1 protein comprising the sequence set forth in SEQ ID NO: 230. In another example, the ASS1 coding sequence is (or comprises a sequence) at least 99%, at least 99.5%, or 100% identical to SEQ ID NO: 228. In another example, the ASS1 coding sequence is (or comprises a sequence) at least 99%, at least 99.5%, or 100% identical to SEQ ID NO: 228 and encodes an ASS1 protein (or an ASS1 protein comprising a sequence) at least 99%, at least 99.5%, or 100% identical to SEQ ID NO: 230. In another example, the ASS1 coding sequence is (or comprises a sequence) at least 99%, at least 99.5%, or 100% identical to SEQ ID NO: 228 and encodes an ASS1 protein comprising the sequence set forth in SEQ ID NO: 230. In another example, the ASS1 coding sequence comprises the sequence set forth in SEQ ID NO: 228. In another example, the ASS1 coding sequence consists essentially of the sequence set forth in SEQ ID NO: 228. In another example, the one of the ASS1 coding sequences consists of the sequence set forth in SEQ ID NO: 228. Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230.

In one example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 706 and 710 (optionally encoding an ASS1 protein comprising the sequence set forth in SEQ ID NO: 230). In another example, the ASS1 coding sequence is (or comprises a sequence) at least 95%, at least 96%, at least 97%, at least 98%, at least 99%, at least 99.5%, or 100% identical to any one of SEQ ID NOS: 706 and 710 (optionally encoding an ASS1 protein comprising the sequence set forth in SEQ ID NO: 230). In another example, the ASS1 coding sequence is (or comprises a sequence) at least 99%, at least 99.5%, or 100% identical to any one of SEQ ID NOS: 706 and 710 (optionally encoding an ASS1 protein comprising the sequence set forth in SEQ ID NO: 230). In another example, the ASS1 coding sequence comprises the sequence set forth in any one of SEQ ID NOS: 706 and 710 (optionally encoding an ASS1 protein comprising the sequence set forth in SEQ ID NO: 230). In another example, the ASS1 coding sequence consists essentially of the sequence set forth in any one of SEQ ID NOS: 706 and 710 (optionally encoding an ASS1 protein comprising the sequence set forth in SEQ ID NO: 230). In another example, the ASS1 coding sequence consists of the sequence set forth in any one of SEQ ID NOS: 706 and 710 (optionally encoding an ASS1 protein comprising the sequence set forth in SEQ ID NO: 230). Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230 (i.e., protein encoded by exons 3-14 of human ASS1).

In one example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 706 (optionally encoding an ASS1 protein comprising the sequence set forth in SEQ ID NO: 230). In another example, the ASS1 coding sequence is (or comprises a sequence) at least 95%, at least 96%, at least 97%, at least 98%, at least 99%, at least 99.5%, or 100% identical to SEQ ID NO: 706 (optionally encoding an ASS1 protein comprising the sequence set forth in SEQ ID NO: 230). In another example, the ASS1 coding sequence is (or comprises a sequence) at least 99%, at least 99.5%, or 100% identical to SEQ ID NO: 706 (optionally encoding an ASS1 protein comprising the sequence set forth in SEQ ID NO: 230). In another example, the ASS1 coding sequence comprises the sequence set forth in SEQ ID NO: 706 (optionally encoding an ASS1 protein comprising the sequence set forth in SEQ ID NO: 230). In another example, the ASS1 coding sequence consists essentially of the sequence set forth in SEQ ID NO: 706 (optionally encoding an ASS1 protein comprising the sequence set forth in SEQ ID NO: 230). In another example, the ASS1 coding sequence consists of the sequence set forth in SEQ ID NO: 706 (optionally encoding an ASS1 protein comprising the sequence set forth in SEQ ID NO: 230). Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230 (i.e., protein encoded by exons 3-14 of human ASS1).

In one example, the ASS1 coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 710 (optionally encoding an ASS1 protein comprising the sequence set forth in SEQ ID NO: 230). In another example, the ASS1 coding sequence is (or comprises a sequence) at least 95%, at least 96%, at least 97%, at least 98%, at least 99%, at least 99.5%, or 100% identical to SEQ ID NO: 710 (optionally encoding an ASS1 protein comprising the sequence set forth in SEQ ID NO: 230). In another example, the ASS1 coding sequence is (or comprises a sequence) at least 99%, at least 99.5%, or 100% identical to SEQ ID NO: 710 (optionally encoding an ASS1 protein comprising the sequence set forth in SEQ ID NO: 230). In another example, the ASS1 coding sequence comprises the sequence set forth in SEQ ID NO: 710 (optionally encoding an ASS1 protein comprising the sequence set forth in SEQ ID NO: 230). In another example, the ASS1 coding sequence consists essentially of the sequence set forth in SEQ ID NO: 710 (optionally encoding an ASS1 protein comprising the sequence set forth in SEQ ID NO: 230). In another example, the ASS1 coding sequence consists of the sequence set forth in SEQ ID NO: 710 (optionally encoding an ASS1 protein comprising the sequence set forth in SEQ ID NO: 230). Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230 (i.e., protein encoded by exons 3-14 of human ASS1).

In one example, the ASS1 coding sequence is (or comprises a sequence) at least 95%, at least 96%, at least 97%, at least 98%, at least 99%, at least 99.5%, or 100% identical to any one of SEQ ID NOS: 705, 709, and 713 (optionally encoding an ASS1 protein comprising the sequence set forth in SEQ ID NO: 230). In another example, the ASS1 coding sequence is (or comprises a sequence) at least 96%, at least 97%, at least 98%, at least 99%, at least 99.5%, or 100% identical to any one of SEQ ID NOS: 705, 709, and 713 (optionally encoding an ASS1 protein comprising the sequence set forth in SEQ ID NO: 230). In another example, the ASS1 coding sequence is (or comprises a sequence) at least 99%, at least 99.5%, or 100% identical to any one of SEQ ID NOS: 705, 709, and 713 (optionally encoding an ASS1 protein comprising the sequence set forth in SEQ ID NO: 230). In another example, the ASS1 coding sequence comprises the sequence set forth in any one of SEQ ID NOS: 705, 709, and 713 (optionally encoding an ASS1 protein comprising the sequence set forth in SEQ ID NO: 230). In another example, the ASS1 coding sequence consists essentially of the sequence set forth in any one of SEQ ID NOS: 705, 709, and 713 (optionally encoding an ASS1 protein comprising the sequence set forth in SEQ ID NO: 230). In another example, the ASS1 coding sequence consists of the sequence set forth in any one of SEQ ID NOS: 705, 709, and 713 (optionally encoding an ASS1 protein comprising the sequence set forth in SEQ ID NO: 230). Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230 (i.e., protein encoded by exons 3-14 of human ASS1).

In one example, the ASS1 coding sequence is (or comprises a sequence) at least 95%, at least 96%, at least 97%, at least 98%, at least 99%, at least 99.5%, or 100% identical to SEQ ID NO: 705 (optionally encoding an ASS1 protein comprising the sequence set forth in SEQ ID NO: 230). In another example, the ASS1 coding sequence is (or comprises a sequence) at least 96%, at least 97%, at least 98%, at least 99%, at least 99.5%, or 100% identical to SEQ ID NO: 705 (optionally encoding an ASS1 protein comprising the sequence set forth in SEQ ID NO: 230). In another example, the ASS1 coding sequence is (or comprises a sequence) at least 99%, at least 99.5%, or 100% identical to SEQ ID NO: 705 (optionally encoding an ASS1 protein comprising the sequence set forth in SEQ ID NO: 230). In another example, the ASS1 coding sequence comprises the sequence set forth in SEQ ID NO: 705 (optionally encoding an ASS1 protein comprising the sequence set forth in SEQ ID NO: 230). In another example, the ASS1 coding sequence consists essentially of the sequence set forth in SEQ ID NO: 705 (optionally encoding an ASS1 protein comprising the sequence set forth in SEQ ID NO: 230). In another example, the ASS1 coding sequence consists of the sequence set forth in SEQ ID NO: 705 (optionally encoding an ASS1 protein comprising the sequence set forth in SEQ ID NO: 230). Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230 (i.e., protein encoded by exons 3-14 of human ASS1).

In one example, the ASS1 coding sequence is (or comprises a sequence) at least 95%, at least 96%, at least 97%, at least 98%, at least 99%, at least 99.5%, or 100% identical to SEQ ID NO: 709 (optionally encoding an ASS1 protein comprising the sequence set forth in SEQ ID NO: 230). In another example, the ASS1 coding sequence is (or comprises a sequence) at least 96%, at least 97%, at least 98%, at least 99%, at least 99.5%, or 100% identical to SEQ ID NO: 709 (optionally encoding an ASS1 protein comprising the sequence set forth in SEQ ID NO: 230). In another example, the ASS1 coding sequence is (or comprises a sequence) at least 99%, at least 99.5%, or 100% identical to SEQ ID NO: 709 (optionally encoding an ASS1 protein comprising the sequence set forth in SEQ ID NO: 230). In another example, the ASS1 coding sequence comprises the sequence set forth in SEQ ID NO: 709 (optionally encoding an ASS1 protein comprising the sequence set forth in SEQ ID NO: 230). In another example, the ASS1 coding sequence consists essentially of the sequence set forth in SEQ ID NO: 709 (optionally encoding an ASS1 protein comprising the sequence set forth in SEQ ID NO: 230). In another example, the ASS1 coding sequence consists of the sequence set forth in SEQ ID NO: 709 (optionally encoding an ASS1 protein comprising the sequence set forth in SEQ ID NO: 230). Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230 (i.e., protein encoded by exons 3-14 of human ASS1).

In one example, the ASS1 coding sequence is (or comprises a sequence) at least 95%, at least 96%, at least 97%, at least 98%, at least 99%, at least 99.5%, or 100% identical to SEQ ID NO: 713 (optionally encoding an ASS1 protein comprising the sequence set forth in SEQ ID NO: 230). In another example, the ASS1 coding sequence is (or comprises a sequence) at least 96%, at least 97%, at least 98%, at least 99%, at least 99.5%, or 100% identical to SEQ ID NO: 713 (optionally encoding an ASS1 protein comprising the sequence set forth in SEQ ID NO: 230). In another example, the ASS1 coding sequence is (or comprises a sequence) at least 99%, at least 99.5%, or 100% identical to SEQ ID NO: 713 (optionally encoding an ASS1 protein comprising the sequence set forth in SEQ ID NO: 230). In another example, the ASS1 coding sequence comprises the sequence set forth in SEQ ID NO: 713 (optionally encoding an ASS1 protein comprising the sequence set forth in SEQ ID NO: 230). In another example, the ASS1 coding sequence consists essentially of the sequence set forth in SEQ ID NO: 713 (optionally encoding an ASS1 protein comprising the sequence set forth in SEQ ID NO: 230). In another example, the ASS1 coding sequence consists of the sequence set forth in SEQ ID NO: 713 (optionally encoding an ASS1 protein comprising the sequence set forth in SEQ ID NO: 230). Optionally, the ASS1 coding sequence encodes an ASS1 protein (or an ASS1 protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence encodes an ASS1 protein (or an ASS1 protein comprising a sequence) at least 95%, at least 96%, at least 97%, at least 98%, at least 99%, at least 99.5%, or 100% identical to SEQ ID NO: 230 (and, e.g., retaining the activity of native ASS1). Optionally, the ASS1 coding sequence in the above examples encodes an ASS1 protein (or an ASS1 protein comprising a sequence) at least 99%, at least 99.5%, or 100% identical to SEQ ID NO: 230 (and, e.g., retaining the activity of native ASS1). Optionally, the ASS1 coding sequence in the above examples encodes an ASS1 protein comprising the sequence set forth in SEQ ID NO: 230. Optionally, the ASS1 coding sequence in the above examples encodes an ASS1 protein consisting essentially of the sequence set forth in SEQ ID NO: 230. Optionally, the ASS1 coding sequence in the above examples encodes an ASS1 protein consisting of the sequence set forth in SEQ ID NO: 230 (i.e., protein encoded by exons 3-14 of human ASS1).

### (4) Vectors

The ASS1 nucleic acid constructs disclosed herein can be provided in a vector for expression or for integration into and expression from a target genomic locus. A vector can comprise additional sequences such as, for example, replication origins, promoters, and genes encoding antibiotic resistance. A vector can also comprise nuclease agent components as disclosed elsewhere herein. For example, a vector can comprise an ASS1 nucleic acid construct, a CRISPR/Cas system (nucleic acids encoding Cas protein and gRNA), one or more components of a CRISPR/Cas system, or a combination thereof (e.g., an ASS1 nucleic acid construct and a gRNA). In some cases, a vector comprising an ASS1 nucleic acid construct does not comprise any components of the nuclease agents described herein (e.g., does not comprise a nucleic acid encoding a Cas protein and does not comprise a nucleic acid encoding a gRNA). Some such vectors comprise homology arms corresponding to target sites in the target genomic locus. Other such vectors do not comprise any homology arms.

Some vectors may be circular. Alternatively, the vector may be linear. The vector can be packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression. Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include 10¹², 10¹³, 10¹⁴, 10¹⁵, and 10¹⁶ vector genomes/mL. Exemplary viral titers (e.g., AAV titers) include about 10¹², about 10¹³, about 10¹⁴, about 10¹⁵, and about 10¹⁶ vector genomes (vg)/mL, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/mL. Other exemplary viral titers (e.g., AAV titers) include about 10¹², about 10¹³, about 10¹⁴, about 10¹⁵, and about 10¹⁶ vector genomes (vg)/kg of body weight, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/kg of body weight. In one example, the viral titer is between about 10¹³ to about 10¹⁴ vg/mL or vg/kg. In another example, the viral titer is between about 10¹² to about 10¹³ vg/mL or vg/kg (e.g., between about 10¹² to about 10¹³ vg/kg). In another example, the viral titer is between about 10¹² to about 10¹⁴ vg/mL or vg/kg (e.g., between about 10¹² to about 10¹⁴ vg/kg). For example, the viral titer can be between about 1.5E12 to about 1.5E13 vg/kg, can be about 1.5E12 vg/kg, or can be about 1.5E13 vg/kg. In another example, the viral titer is about 2E13 vg/mL or vg/kg.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

Some non-limiting examples of ITRs that can be used include ITRs comprising, consisting essentially of, or consisting of SEQ ID NO: 241, SEQ ID NO: 242, or SEQ ID NO: 243. Other examples of ITRs comprise one or more mutations compared to SEQ ID NO: 241, SEQ ID NO: 242, or SEQ ID NO: 243 and can be at least 90%, at least 91%, at least 92%, at least 93%, at least 94%, at least 95%, at least 96%, at least 97%, at least 98%, or at least 99% identical to SEQ ID NO: 241, SEQ ID NO: 242, or SEQ ID NO: 243. In some rAAV genomes disclosed herein, the ASS1 nucleic acid construct is flanked on both sides by the same ITR (i.e., the ITR on the 5′ end, and the reverse complement of the ITR on the 3′ end). In one example, the ITR on each end can comprise, consist essentially of, or consist of SEQ ID NO: 241. In another example, the ITR on each end can comprise, consist essentially of, or consist of SEQ ID NO: 242. In one example, the ITR on at least one end comprises, consists essentially of, or consists of SEQ ID NO: 243. In one example, the ITR on the 5′ end comprises, consists essentially of, or consists of SEQ ID NO: 243. In one example, the ITR on the 3′ end comprises, consists essentially of, or consists of SEQ ID NO: 243. In one example, the ITR on each end can comprise, consist essentially of, or consist of SEQ ID NO: 243. In one example, the ITR on at least one end comprises, consists essentially of, or consists of SEQ ID NO: 241. In one example, the ITR on the 5′ end comprises, consists essentially of, or consists of SEQ ID NO: 241. In one example, the ITR on the 3′ end comprises, consists essentially of, or consists of SEQ ID NO: 241. In one example, the ITR on each end can comprise, consist essentially of, or consist of SEQ ID NO: 241. In other rAAV genomes disclosed herein, the ASS1 nucleic acid construct is flanked by different ITRs on each end. In one example, the ITR on one end comprises, consists essentially of, or consists of SEQ ID NO: 241, and the ITR on the other end comprises, consists essentially of, or consists of SEQ ID NO: 242. In another example, the ITR on one end comprises, consists essentially of, or consists of SEQ ID NO: 241, and the ITR on the other end comprises, consists essentially of, or consists of SEQ ID NO: 243. In one example, the ITR on one end comprises, consists essentially of, or consists of SEQ ID NO: 242, and the ITR on the other end comprises, consists essentially of, or consists of SEQ ID NO: 243.

The specific serotype of a recombinant AAV vector influences its in-vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8 and rAAV5, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

When specific rAAVs comprising specific sequences (e.g., specific bidirectional construct sequences or specific unidirectional construct sequences) are disclosed herein, they are meant to encompass the sequence disclosed or the reverse complement of the sequence. For example, if a bidirectional or unidirectional construct disclosed herein consists of the hypothetical sequence 5′-CTGGACCGA-3′, it is also meant to encompass the reverse complement of that sequence (5′-TCGGTCCAG-3′). Likewise, when rAAVs comprising bidirectional or unidirectional construct elements in a specific 5′ to 3′ order are disclosed herein, they are also meant to encompass the reverse complement of the order of those elements. For example, if an rAAV is disclosed herein that comprises a bidirectional construct that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., Ling et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. An “AAV vector” as used herein refers to an AAV vector comprising a heterologous sequence not of AAV origin (i.e., a nucleic acid sequence heterologous to AAV), typically comprising a sequence encoding a heterologous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector. In another specific example, the AAV vector comprising the nucleic acid construct can be recombinant AAV5 (rAAV5). A rAAV5 vector as described herein is one in which the capsid is from AAV5. For example, an AAV vector using ITRs from AAV2 and a capsid of AAV5 is considered herein to be a rAAV5 vector.

Tropism can be further refined through pseudotyping, which is the mixing of a capsid and a genome from different viral serotypes. For example, AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

The vector (e.g., AAV such as recombinant AAV8 or recombinant AAV5) can be formulated, for example, in 10 mM sodium phosphate, 180 mM sodium chloride, and 0.005% poloxamer 188, at pH 7.3.

### B. Nuclease Agents and CRISPR/Cas Systems

The methods and compositions disclosed herein can utilize nuclease agents such as Clustered Regularly Interspersed Short Palindromic Repeats (CRISPR)/CRISPR-associated (Cas) systems, zinc finger nuclease (ZFN) systems, or Transcription Activator-Like Effector Nuclease (TALEN) systems or components of such systems to modify a target genomic locus in a target gene such as an ASS1 gene for insertion of an ASS1 nucleic acid construct as disclosed herein. Generally, the nuclease agents involve the use of engineered cleavage systems to induce a double strand break or a nick (i.e., a single strand break) in a nuclease target site. Cleavage or nicking can occur through the use of specific nucleases such as engineered ZFNs, TALENs, or CRISPR/Cas systems with an engineered guide RNA to guide specific cleavage or nicking of the nuclease target site. Any nuclease agent that induces a nick or double-strand break at a desired target sequence can be used in the methods and compositions disclosed herein. The nuclease agent can be used to create a site of insertion at a desired locus (target gene) within a host genome, at which site the ASS1 nucleic acid construct is inserted to express ASS1. The ASS1 may be heterologous with respect to its insertion site or locus (target gene), such as a safe harbor locus from which ASS1 is not normally expressed. Alternatively, the ASS1 may be non-heterologous with respect to its insertion site, such as insertion into the endogenous ASS1 locus to correct a defective ASS1 gene.

In one example, the nuclease agent is a CRISPR/Cas system. In another example, the nuclease agent comprises one or more ZFNs. In yet another example, the nuclease agent comprises one or more TALENs. In a specific example, the CRISPR/Cas systems or components of such systems target an ASS1 gene or locus (e.g., ASS1 genomic locus) within a cell, or intron 1 or intron 2 of an ASS1 gene or locus within a cell. In a more specific example, the CRISPR/Cas systems or components of such systems target a human ASS1 gene or locus or intron 1 or intron 2 of a human ASS1 gene or locus (e.g., intron 2 of a human ASS1 gene or locus or intron 1 of a human ASS1 gene or locus) within a cell.

CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed binding or cleavage of nucleic acids. A CRISPR/Cas system targeting an ASS1 gene or locus comprises a Cas protein (or a nucleic acid encoding the Cas protein) and one or more guide RNAs (or DNAs encoding the one or more guide RNAs), with each of the one or more guide RNAs targeting a different guide RNA target sequence in the target genomic locus (e.g., ASS1 gene or locus).

CRISPR/Cas systems used in the compositions and methods disclosed herein can be non-naturally occurring. A non-naturally occurring system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

### (1) Target Genomic Loci and Argininosuccinate Synthase (ASS1)

Any target genomic locus capable of expressing a gene can be used, such as the endogenous ASS1 locus, including human ASS1 intron 2, human ASS1 intron 1, or mouse Ass1 intron 1. Additional target genomic loci can be used, such as a safe harbor locus, including human ALB or mouse Alb. The nucleic acid construct can be integrated into any part of the target genomic locus. For example, the nucleic acid construct can be inserted into an intron or an exon of a target genomic locus or can replace one or more introns and/or exons of a target genomic locus. In a specific example, the nucleic acid construct can be integrated into an intron of the target genomic locus, such as an intron of the target genomic locus (e.g., human ASS1 intron 2, human ASS1 intron 1, or mouse Ass1 intron 1). In a more specific example, the nucleic acid construct can be integrated into human ASS1 intron 2. In another example, the nucleic acid construct can be integrated into the first intron of a safe harbor gene (e.g., intron 1 of human ALB or mouse Alb). See, e.g., WO 2020/082042, US 2020/0270617, WO 2020/082041, US 2020/0268906, WO 2020/082046, and US 2020/0289628, each of which is herein incorporated by reference in its entirety for all purposes. Constructs integrated into a target genomic locus can be operably linked to an endogenous promoter at the target genomic locus (e.g., the endogenous ASS1 promoter).

Interactions between integrated exogenous DNA and a host genome can limit the reliability and safety of integration and can lead to overt phenotypic effects that are not due to the targeted genetic modification but are instead due to unintended effects of the integration on surrounding endogenous genes. For example, randomly inserted transgenes can be subject to position effects and silencing, making their expression unreliable and unpredictable. Likewise, integration of exogenous DNA into a chromosomal locus can affect surrounding endogenous genes and chromatin, thereby altering cell behavior and phenotypes. Safe harbor loci include chromosomal loci where transgenes or other exogenous nucleic acid inserts can be stably and reliably expressed in all tissues of interest without overtly altering cell behavior or phenotype (i.e., without any deleterious effects on the host cell). See, e.g., Sadelain et al. (2012) Nat. Rev. Cancer 12:51-58, herein incorporated by reference in its entirety for all purposes. For example, the safe harbor locus can be one in which expression of the inserted gene sequence is not perturbed by any read-through expression from neighboring genes. For example, safe harbor loci can include chromosomal loci where exogenous DNA can integrate and function in a predictable manner without adversely affecting endogenous gene structure or expression. Safe harbor loci can include extragenic regions or intragenic regions such as, for example, loci within genes that are non-essential, dispensable, or able to be disrupted without overt phenotypic consequences.

Such safe harbor loci can offer an open chromatin configuration in all tissues and can be ubiquitously expressed during embryonic development and in adults. See, e.g., Zambrowicz et al. (1997) Proc. Natl. Acad. Sci. U.S.A. 94:3789-3794, herein incorporated by reference in its entirety for all purposes. In addition, the safe harbor loci can be targeted with high efficiency, and safe harbor loci can be disrupted with no overt phenotype. Examples of safe harbor loci include ALB, CCR5, HPRT, AAVS1, and Rosa26. See, e.g., U.S. Pat. Nos. 7,888,121; 7,972,854; 7,914,796; 7,951,925; 8,110,379; 8,409,861; 8,586,526; and US Patent Publication Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; 2013/0177960; and 2013/0122591, each of which is herein incorporated by reference in its entirety for all purposes.

In a specific example, a safe harbor locus is a locus within the genome wherein a gene may be inserted without significant deleterious effects on the host cell such as a hepatocyte (e.g., without causing apoptosis, necrosis, and/or senescence, or without causing more than 5%, 10%, 15%, 20%, 25%, 30%, or 40% apoptosis, necrosis, and/or senescence as compared to a control cell). The safe harbor locus can allow overexpression of an exogenous gene without significant deleterious effects on the host cell such as a hepatocyte (e.g., without causing apoptosis, necrosis, and/or senescence, or without causing more than 5%, 10%, 15%, 20%, 25%, 30%, or 40% apoptosis, necrosis, and/or senescence as compared to a control cell). A desirable safe harbor locus may be one in which expression of the inserted gene sequence is not perturbed by read-through expression from neighboring genes. The safe harbor may be a human safe harbor (e.g., for a liver tissue or hepatocyte host cell).

In a specific example, the target genomic locus is an ASS1 locus, such as intron 2 of an ASS1 locus or intron 1 of an ASS1 locus. In a more specific example, the target genomic locus is a human ASS1 locus, such as intron 2 of a human ASS1 locus (e.g., SEQ ID NO: 4). In another example, the target genomic locus is intron 1 of a human ASS1 locus. In another example, the target genomic locus is an ASS1 locus, such as intron 1 of an ASS1 locus. In a more specific example, the target genomic locus is a mouse Ass1 locus, such as intron 1 of a mouse Ass1 locus (e.g., SEQ ID NO: 5). Alternatively, the target genomic locus can be intron 1 of a human ASS1 locus. In another example, the target genomic locus is an ALB locus, such as intron 1 of an ALB locus. In a more specific example, the target genomic locus is a human ALB locus, such as intron 1 of a human ALB locus. Alternatively, the target genomic locus can be intron 1 of a mouse Alb locus.

### (2) Cas Proteins

Cas proteins generally comprise at least one RNA recognition or binding domain that can interact with guide RNAs. Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus.

Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (e.g., assigned UniProt accession number Q99ZW2) is an exemplary Cas9 protein. An exemplary SpCas9 protein sequence is set forth in SEQ ID NO: 9 (encoded by the DNA sequence set forth in SEQ ID NO: 10). An exemplary SpCas9 mRNA (cDNA) sequence is set forth in SEQ ID NO: 11. Smaller Cas9 proteins (e.g., Cas9 proteins whose coding sequences are compatible with the maximum AAV packaging capacity when combined with a guide RNA coding sequence and regulatory elements for the Cas9 and guide RNA, such as SaCas9 and CjCas9 and Nme2Cas9) are other exemplary Cas9 proteins. For example, Cas9 from S. aureus (SaCas9) (e.g., assigned UniProt accession number J7RUA5) is another exemplary Cas9 protein. Likewise, Cas9 from Campylobacter jejuni (CjCas9) (e.g., assigned UniProt accession number Q0P897) is another exemplary Cas9 protein. See, e.g., Kim et al. (2017) Nat. Commun. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73(4):714-726, herein incorporated by reference in its entirety for all purposes. Cas9 proteins from Streptococcus thermophilus (e.g., Streptococcus thermophilus LMD-9 Cas9 encoded by the CRISPR1 locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28(7):247-261, herein incorporated by reference in its entirety for all purposes. Examples of Cas9 coding sequences, Cas9 mRNAs, and Cas9 protein sequences are provided in WO 2013/176772, WO 2014/065596, WO 2016/106121, WO 2019/067910, WO 2020/082042, US 2020/0270617, WO 2020/082041, US 2020/0268906, WO 2020/082046, and US 2020/0289628, each of which is herein incorporated by reference in its entirety for all purposes. Specific examples of ORFs and Cas9 amino acid sequences are provided in Table 30 at paragraph [0449] WO 2019/067910, and specific examples of Cas9 mRNAs and ORFs are provided in paragraphs [0214]-[0234] of WO 2019/067910. See also WO 2020/082046 A2 (pp. 84-85) and Table 24 in WO 2020/069296, each of which is herein incorporated by reference in its entirety for all purposes. An exemplary SpCas9 protein sequence comprises, consists essentially of, or consists of SEQ ID NO: 12. An exemplary SpCas9 mRNA sequence encoding that SpCas9 protein sequence comprises, consists essentially of, or consists of SEQ ID NO: 13. Another exemplary SpCas9 mRNA sequence encoding that SpCas9 protein sequence comprises, consists essentially of, or consists of SEQ ID NO: 249. Another exemplary SpCas9 mRNA sequence encoding that SpCas9 protein sequence comprises SEQ ID NO: 250. An exemplary SpCas9 coding sequence comprises, consists essentially of, or consists of SEQ ID NO: 251.

Another example of a Cas protein is a Cpf1 (CRISPR from Prevotella and Francisella 1) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163(3):759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC20171, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number A0Q7Q2) is an exemplary Cpf1 protein.

Another example of a Cas protein is CasX (Cas12e). CasX is an RNA-guided DNA endonuclease that generates a staggered double-strand break in DNA. CasX is less than 1000 amino acids in size. Exemplary CasX proteins are from Deltaproteobacteria (DpbCasX or DpbCas12e) and Planctomycetes (PlmCasX or PlmCas12e). Like Cpf1, CasX uses a single RuvC active site for DNA cleavage. See, e.g., Liu et al. (2019) Nature 566(7743):218-223, herein incorporated by reference in its entirety for all purposes.

Another example of a Cas protein is CasΦ (CasPhi or Cas12j), which is uniquely found in bacteriophages. CasΦ is less than 1000 amino acids in size (e.g., 700-800 amino acids). CasΦ cleavage generates staggered 5′ overhangs. A single RuvC active site in CasΦ is capable of crRNA processing and DNA cutting. See, e.g., Pausch et al. (2020) Science 369(6501):333-337, herein incorporated by reference in its entirety for all purposes.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529(7587):490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351(6268):84-88, herein incorporated by reference in its entirety for all purposes. Other SpCas9 variants include K855A and K810A/K1003A/R1060A. These and other modified Cas proteins are reviewed, e.g., in Cebrian-Serrano and Davies (2017) Mamm. Genome 28(7):247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Likewise, CasX and CasΦ generally comprise a single RuvC-like domain that cleaves both strands of a target DNA. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337(6096):816-821, herein incorporated by reference in its entirety for all purposes.

One or more of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If none of the nuclease domains is deleted or mutated in a Cas9 protein, the Cas9 protein will retain double-strand-break-inducing activity. An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39(21):9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) or a substitution at position D10 (e.g., D10A substitution) to generate a Cas nickase. See, e.g., WO 2016/106236, herein incorporated by reference in its entirety for all purposes. Examples of inactivating mutations in the catalytic domains of Nme2Cas9 are also known (e.g., D16A or H588A). Examples of inactivating mutations in the catalytic domains of St1Cas9 are also known (e.g., D9A, D598A, H599A, or N622A). Examples of inactivating mutations in the catalytic domains of St3Cas9 are also known (e.g., D10A or N870A). Examples of inactivating mutations in the catalytic domains of CjCas9 are also known (e.g., combination of D8A or H559A). Examples of inactivating mutations in the catalytic domains of FnCas9 and RHA FnCas9 are also known (e.g., N995A).

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example, one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes.

Examples of inactivating mutations in the catalytic domains of CasX proteins are also known. With reference to CasX proteins from Deltaproteobacteria, D672A, E769A, and D935A (individually or in combination) or corresponding positions in other CasX orthologs are inactivating. See, e.g., Liu et al. (2019) Nature 566(7743):218-223, herein incorporated by reference in its entirety for all purposes.

Examples of inactivating mutations in the catalytic domains of CasΦ proteins are also known. For example, D371A and D394A, alone or in combination, are inactivating mutations. See, e.g., Pausch et al. (2020) Science 369(6501):333-337, herein incorporated by reference in its entirety for all purposes.

Cas proteins can also be operably linked to heterologous polypeptides as fusion proteins. For example, a Cas protein can be fused to a cleavage domain. See WO 2014/089290, herein incorporated by reference in its entirety for all purposes Cas proteins can also be fused to a heterologous polypeptide providing increased or decreased stability. The fused domain or heterologous polypeptide can be located at the N-terminus, the C-terminus, or internally within the Cas protein.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282(8):5101-5105, herein incorporated by reference in its entirety for all purposes. Such subcellular localization signals can be located at the N-terminus, the C-terminus, or anywhere within the Cas protein. An NLS can comprise a stretch of basic amino acids, and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

A Cas protein may, for example, be fused with 1-10 NLSs (e.g., fused with 1-5 NLSs or fused with one NLS. Where one NLS is used, the NLS may be linked at the N-terminus or the C-terminus of the Cas protein sequence. It may also be inserted within the Cas protein sequence. Alternatively, the Cas protein may be fused with more than one NLS. For example, the Cas protein may be fused with 2, 3, 4, or 5 NLSs. In a specific example, the Cas protein may be fused with two NLSs. In certain circumstances, the two NLSs may be the same (e.g., two SV40 NLSs) or different. For example, the Cas protein can be fused to two SV40 NLS sequences linked at the carboxy terminus. Alternatively, the Cas protein may be fused with two NLSs, one linked at the N-terminus and one at the C-terminus. In other examples, the Cas protein may be fused with 3 NLSs or with no NLS. The NLS may be a monopartite sequence, such as, e.g., the SV40 NLS, PKKKRKV (SEQ ID NO: 14) or PKKKRRV (SEQ ID NO: 15). The NLS may be a bipartite sequence, such as the NLS of nucleoplasmin, KRPAATKKAGQAKKKK (SEQ ID NO: 16). In a specific example, a single PKKKRKV (SEQ ID NO: 14) NLS may be linked at the C-terminus of the Cas protein. One or more linkers are optionally included at the fusion site.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreenl), yellow fluorescent proteins (e.g., YFP, eYFP, Citrine, Venus, YPet, PhiYFP, ZsYellowl), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

Cas proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the Cas protein. In one example, the labeled nucleic acid is tethered to the C-terminus or the N-terminus of the Cas protein. Likewise, the Cas protein can be tethered to the 5′ end, the 3′ end, or to an internal region within the labeled nucleic acid. That is, the labeled nucleic acid can be tethered in any orientation and polarity. For example, the Cas protein can be tethered to the 5′ end or the 3′ end of the labeled nucleic acid.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence. When a nucleic acid encoding the Cas protein is introduced into the cell, the Cas protein can be transiently, conditionally, or constitutively expressed in the cell.

Nucleic acids encoding Cas proteins can be stably integrated in the genome of a cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding a gRNA. Alternatively, it can be in a vector or plasmid that is separate from the vector comprising the DNA encoding the gRNA. Promoters that can be used in an expression construct include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allow for the generation of compact expression cassettes to facilitate delivery.

Different promoters can be used to drive Cas expression or Cas9 expression. In some methods, small promoters are used so that the Cas or Cas9 coding sequence can fit into an AAV construct. For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., AAV2-mediated delivery, AAV5-mediated delivery, AAV8-mediated delivery, or AAV7m8-mediated delivery). For example, the nuclease agent can be CRISPR/Cas9, and a Cas9 mRNA and a gRNA targeting an intron 2 of an endogenous human ASS1 locus can be delivered via LNP-mediated delivery or AAV-mediated delivery. As another example, the nuclease agent can be CRISPR/Cas9, and a Cas9 mRNA and a gRNA targeting an intron 1 of an endogenous human ASS1 locus can be delivered via LNP-mediated delivery or AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

Cas proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding Cas proteins can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding Cas proteins can also be polyadenylated (to comprise a poly(A) tail). mRNA encoding Cas proteins can also be modified to include pseudouridine (e.g., can be fully substituted with pseudouridine). As another example, capped and polyadenylated Cas mRNA containing N1-methyl-pseudouridine can be used. mRNA encoding Cas proteins can also be modified to include N1-methyl-pseudouridine (e.g., can be fully substituted with N1-methyl-pseudouridine). As another example, Cas mRNA fully substituted with pseudouridine can be used (i.e., all standard uracil residues are replaced with pseudouridine, a uridine isomer in which the uracil is attached with a carbon-carbon bond rather than nitrogen-carbon). As another example, Cas mRNA fully substituted with N1-methyl-pseudouridine can be used (i.e., all standard uracil residues are replaced with N1-methyl-pseudouridine). Likewise, Cas mRNAs can be modified by depletion of uridine using synonymous codons. For example, capped and polyadenylated Cas mRNA fully substituted with pseudouridine can be used. For example, capped and polyadenylated Cas mRNA fully substituted with N1-methyl-pseudouridine can be used.

Cas mRNAs can comprise a modified uridine at least at one, a plurality of, or all uridine positions. The modified uridine can be a uridine modified at the 5 position (e.g., with a halogen, methyl, or ethyl). The modified uridine can be a pseudouridine modified at the 1 position (e.g., with a halogen, methyl, or ethyl). The modified uridine can be, for example, pseudouridine, N1-methyl-pseudouridine, 5-methoxyuridine, 5-iodouridine, or a combination thereof. In some examples, the modified uridine is 5-methoxyuridine. In some examples, the modified uridine is 5-iodouridine. In some examples, the modified uridine is pseudouridine. In some examples, the modified uridine is N1-methyl-pseudouridine. In some examples, the modified uridine is a combination of pseudouridine and N1-methyl-pseudouridine. In some examples, the modified uridine is a combination of pseudouridine and 5-methoxyuridine. In some examples, the modified uridine is a combination of N1-methyl pseudouridine and 5-methoxyuridine. In some examples, the modified uridine is a combination of 5-iodouridine and N1-methyl-pseudouridine. In some examples, the modified uridine is a combination of pseudouridine and 5-iodouridine. In some examples, the modified uridine is a combination of 5-iodouridine and 5-methoxyuridine.

Cas mRNAs disclosed herein can also comprise a 5′ cap, such as a Cap0, Cap1, or Cap2. A 5′ cap is generally a 7-methylguanine ribonucleotide (which may be further modified, e.g., with respect to ARCA) linked through a 5′-triphosphate to the 5′ position of the first nucleotide of the 5′-to-3′ chain of the mRNA (i.e., the first cap-proximal nucleotide). In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc. Natl. Acad. Sci. U.S.A. 111(33):12025-30 and Abbas et al. (2017) Proc. Natl. Acad. Sci. U.S.A. 114(11):E2106-E2115, each of which is herein incorporated by reference in its entirety for all purposes. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as non-self by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al. (2001) RNA 7:1486-1495, herein incorporated by reference in its entirety for all purposes.

CleanCap™ AG (m7G(5′)ppp(5′)(2′OMeA)pG; TriLink Biotechnologies Cat. No. N-7113) or CleanCap™ GG (m7G(5′)ppp(5′)(2′OmeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively.

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo and Moss (1990) Proc. Natl. Acad. Sci. U.S.A. 87:4023-4027 and Mao and Shuman (1994) J. Biol. Chem. 269:24472-24479, each of which is herein incorporated by reference in its entirety for all purposes.

Cas mRNAs can further comprise a poly-adenylated (poly-A or poly(A) or poly-adenine) tail. The poly-A tail can, for example, comprise at least 20, at least 30, at least 40, at least 50, at least 60, at least 70, at least 80, at least 90, or at least 100 adenines, and optionally up to 300 adenines. For example, the poly-A tail can comprise 95, 96, 97, 98, 99, or 100 adenine nucleotides.

### (3) Guide RNAs

A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1 and CasΦ, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a gRNA is a S. pyogenes Cas9 gRNA or an equivalent thereof. In some of the methods and compositions disclosed herein, a gRNA is a S. aureus Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail (e.g., for use with S. pyogenes Cas9), located downstream (3′) of the DNA-targeting segment, comprises, consists essentially of, or consists of GUUUUAGAGCUAUGCU (SEQ ID NO: 17) or GUUUUAGAGCUAUGCUGUUUUG (SEQ ID NO: 18). Any of the DNA-targeting segments disclosed herein can be joined to the 5′ end of SEQ ID NO: 17 or 18 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences (e.g., for use with S. pyogenes Cas9) comprise, consist essentially of, or consist of any one of

(SEQ ID NO: 19)
AGCAUAGCAAGUUAAAAUAAGGCUAGUCCGUUAUCAACUUGAAAAAGUG
GCACCGAGUCGGUGCUUU,
(SEQ ID NO: 20)
AAACAGCAUAGCAAGUUAAAAUAAGGCUAGUCCGUUAUCAACUUGAAAA
AGUGGCACCGAGUCGGUGCUUUU,
or
(SEQ ID NO: 21)
GUUGGAACCAUUCAAAACAGCAUAGCAAGUUAAAAUAAGGCUAGUCCGU
UAUCAACUUGAAAAAGUGGCACCGAGUCGGUGC.

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339(6121):823-826; Jinek et al. (2012) Science 337(6096):816-821; Hwang et al. (2013) Nat. Biotechnol. 31(3):227-229; Jiang et al. (2013) Nat. Biotechnol. 31(3):233-239; and Cong et al. (2013) Science 339(6121):819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, about 96%, about 97%, about 98%, about 99%, or about 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

As one example, a guide RNA targeting intron 2 of a human ASS1 gene can comprise a DNA-targeting segment (i.e., guide sequence) comprising, consisting essentially of, or consisting of the sequence (DNA-targeting segment) set forth in any one of SEQ ID NOS: 31-118. Alternatively, a guide RNA targeting intron 2 of a human ASS1 gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 31-118. Alternatively, a guide RNA targeting intron 2 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 31-118. Alternatively, a guide RNA targeting intron 2 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 31-118. Alternatively, a guide RNA targeting intron 2 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 31-118. Alternatively, a guide RNA targeting intron 2 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 31-118. Alternatively, a guide RNA targeting intron 2 of a human ASS1 gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 31-118. Alternatively, a guide RNA targeting intron 2 of a human ASS1 gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 31-118.

As one example, a guide RNA targeting intron 2 of a cynomolgus macaque ASS1 gene can comprise a DNA-targeting segment (i.e., guide sequence) comprising, consisting essentially of, or consisting of the sequence (DNA-targeting segment) set forth in SEQ ID NO: 714. Alternatively, a guide RNA targeting intron 2 of a cynomolgus macaque ASS1 gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 714. Alternatively, a guide RNA targeting intron 2 of a cynomolgus macaque ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 714. Alternatively, a guide RNA targeting intron 2 of a cynomolgus macaque ASS1 gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 714. Alternatively, a guide RNA targeting intron 2 of a cynomolgus macaque ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 714. Alternatively, a guide RNA targeting intron 2 of a cynomolgus macaque ASS1 gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 714. Alternatively, a guide RNA targeting intron 2 of a cynomolgus macaque ASS1 gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 714. Alternatively, a guide RNA targeting intron 2 of a cynomolgus macaque ASS1 gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 714.

As one example, a guide RNA targeting intron 1 of a human ASS1 gene can comprise a DNA-targeting segment (i.e., guide sequence) comprising, consisting essentially of, or consisting of the sequence (DNA-targeting segment) set forth in any one of SEQ ID NOS: 430-517. Alternatively, a guide RNA targeting intron 1 of a human ASS1 gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 430-517. Alternatively, a guide RNA targeting intron 1 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 430-517. Alternatively, a guide RNA targeting intron 1 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 430-517. Alternatively, a guide RNA targeting intron 1 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 430-517. Alternatively, a guide RNA targeting intron 1 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 430-517. Alternatively, a guide RNA targeting intron 1 of a human ASS1 gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 430-517. Alternatively, a guide RNA targeting intron 1 of a human ASS1 gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 430-517.

As another example, a guide RNA targeting intron 1 of a mouse Ass1 gene can comprise a DNA-targeting segment (i.e., guide sequence) comprising, consisting essentially of, or consisting of the sequence (DNA-targeting segment) set forth in any one of SEQ ID NOS: 207-212. Alternatively, a guide RNA targeting intron1 of a mouse Ass1 gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 207-212. Alternatively, a guide RNA targeting intron1 of a mouse Ass1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 207-212. Alternatively, a guide RNA targeting intron1 of a mouse Ass1 gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 207-212. Alternatively, a guide RNA targeting intron1 of a mouse Ass1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 207-212. Alternatively, a guide RNA targeting intron1 of a mouse Ass1 gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 207-212. Alternatively, a guide RNA targeting intron1 of a mouse Ass1 gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 207-212. Alternatively, a guide RNA targeting intron1 of a mouse Ass1 gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 207-212.

As another example, a guide RNA targeting intron 2 of a human ASS1 gene can comprise a DNA-targeting segment (i.e., guide sequence) comprising, consisting essentially of, or consisting of the sequence (DNA-targeting segment) set forth in any one of SEQ ID NOS: 58, 60, 67, 73, 78, 89, 92, and 114. Alternatively, a guide RNA targeting intron 2 of a human ASS1 gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 58, 60, 67, 73, 78, 89, 92, and 114. Alternatively, a guide RNA targeting intron 2 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 58, 60, 67, 73, 78, 89, 92, and 114. Alternatively, a guide RNA targeting intron 2 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 58, 60, 67, 73, 78, 89, 92, and 114. Alternatively, a guide RNA targeting intron 2 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 58, 60, 67, 73, 78, 89, 92, and 114. Alternatively, a guide RNA targeting intron 2 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 58, 60, 67, 73, 78, 89, 92, and 114. Alternatively, a guide RNA targeting intron 2 of a human ASS1 gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 58, 60, 67, 73, 78, 89, 92, and 114. Alternatively, a guide RNA targeting intron 2 of a human ASS1 gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 58, 60, 67, 73, 78, 89, 92, and 114.

As another example, a guide RNA targeting intron 2 of a human ASS1 gene can comprise a DNA-targeting segment (i.e., guide sequence) comprising, consisting essentially of, or consisting of the sequence (DNA-targeting segment) set forth in any one of SEQ ID NOS: 78, 60, and 89. Alternatively, a guide RNA targeting intron 2 of a human ASS1 gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 78, 60, and 89. Alternatively, a guide RNA targeting intron 2 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 78, 60, and 89. Alternatively, a guide RNA targeting intron 2 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 78, 60, and 89. Alternatively, a guide RNA targeting intron 2 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 78, 60, and 89. Alternatively, a guide RNA targeting intron 2 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 78, 60, and 89. Alternatively, a guide RNA targeting intron 2 of a human ASS1 gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 78, 60, and 89. Alternatively, a guide RNA targeting intron 2 of a human ASS1 gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 78, 60, and 89.

As another example, a guide RNA targeting intron 2 of a human ASS1 gene can comprise a DNA-targeting segment (i.e., guide sequence) comprising, consisting essentially of, or consisting of the sequence (DNA-targeting segment) set forth in SEQ ID NO: 78. Alternatively, a guide RNA targeting intron 2 of a human ASS1 gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 78. Alternatively, a guide RNA targeting intron 2 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 78. Alternatively, a guide RNA targeting intron 2 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 78. Alternatively, a guide RNA targeting intron 2 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 78. Alternatively, a guide RNA targeting intron 2 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 78. Alternatively, a guide RNA targeting intron 2 of a human ASS1 gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 78. Alternatively, a guide RNA targeting intron 2 of a human ASS1 gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 78.

As another example, a guide RNA targeting intron 2 of a human ASS1 gene can comprise a DNA-targeting segment (i.e., guide sequence) comprising, consisting essentially of, or consisting of the sequence (DNA-targeting segment) set forth in SEQ ID NO: 60. Alternatively, a guide RNA targeting intron 2 of a human ASS1 gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 60. Alternatively, a guide RNA targeting intron 2 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 60. Alternatively, a guide RNA targeting intron 2 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 60. Alternatively, a guide RNA targeting intron 2 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 60. Alternatively, a guide RNA targeting intron 2 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 60. Alternatively, a guide RNA targeting intron 2 of a human ASS1 gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 60. Alternatively, a guide RNA targeting intron 2 of a human ASS1 gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 60.

As another example, a guide RNA targeting intron 2 of a human ASS1 gene can comprise a DNA-targeting segment (i.e., guide sequence) comprising, consisting essentially of, or consisting of the sequence (DNA-targeting segment) set forth in SEQ ID NO: 89. Alternatively, a guide RNA targeting intron 2 of a human ASS1 gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 89. Alternatively, a guide RNA targeting intron 2 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 89. Alternatively, a guide RNA targeting intron 2 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 89. Alternatively, a guide RNA targeting intron 2 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 89. Alternatively, a guide RNA targeting intron 2 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 89. Alternatively, a guide RNA targeting intron 2 of a human ASS1 gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 89. Alternatively, a guide RNA targeting intron 2 of a human ASS1 gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 89.

As another example, a guide RNA targeting intron 2 of a human ASS1 gene can comprise a DNA-targeting segment (i.e., guide sequence) comprising, consisting essentially of, or consisting of the sequence (DNA-targeting segment) set forth in SEQ ID NO: 58. Alternatively, a guide RNA targeting intron 2 of a human ASS1 gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 58. Alternatively, a guide RNA targeting intron 2 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 58. Alternatively, a guide RNA targeting intron 2 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 58. Alternatively, a guide RNA targeting intron 2 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 58. Alternatively, a guide RNA targeting intron 2 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 58. Alternatively, a guide RNA targeting intron 2 of a human ASS1 gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 58. Alternatively, a guide RNA targeting intron 2 of a human ASS1 gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 58.

As another example, a guide RNA targeting intron 2 of a human ASS1 gene can comprise a DNA-targeting segment (i.e., guide sequence) comprising, consisting essentially of, or consisting of the sequence (DNA-targeting segment) set forth in SEQ ID NO: 67. Alternatively, a guide RNA targeting intron 2 of a human ASS1 gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 67. Alternatively, a guide RNA targeting intron 2 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 67. Alternatively, a guide RNA targeting intron 2 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 67. Alternatively, a guide RNA targeting intron 2 of a human ASS1 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 67. Alternatively, a guide RNA targeting intron 2 of a human ASS1 gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 67. Alternatively, a guide RNA targeting intron 2 of a human ASS1 gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 67. Alternatively, a guide RNA targeting intron 2 of a human ASS1 gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 67.

TABLE 2
Human ASS1 Intron 2 Guide Sequences.

| Guide RNA ID# | Guide Sequence | SEQ ID NO: |
|---|---|---|
| G035811 | GAGUGCGCUUUCAGCAGCGC | 31 |
| G035812 | CCUCUACACUCUAAUUUACG | 32 |
| G035813 | CAGCGGGGGUGGCCCCGUAU | 33 |
| G035814 | AGCUAGCUGAGACCUAUACG | 34 |
| G035815 | CCACGUAAAUUAGAGUGUAG | 35 |
| G035816 | CACUCGAAUCUUCAACACUC | 36 |
| G035817 | ACUCGAAUCUUCAACACUCA | 37 |
| G035818 | GUUUCACAGAACGGUGCAUC | 38 |
| G035819 | GGUUGCCACAACUACCAGGU | 39 |
| G035820 | UGGCCUUGUUCCCGUGUGGC | 40 |
| G035821 | CGGCCAGCCACACGGGAACA | 41 |
| G035822 | AAACAAGGCAUCACAUAGCG | 42 |
| G035823 | GUAUAGGUCUCAGCUAGCUG | 43 |
| G035824 | GGUGCCUUGAUUGUGAAUCU | 44 |
| G035825 | GUGCAUCAGGAUUGAUUAGC | 45 |
| G035826 | GGGGUUCCCAAUGACUUUUC | 46 |
| G035827 | UCAGCUAGCUGAGACCUAUA | 47 |
| G035828 | GAUGCACCGUUCUGUGAAAC | 48 |
| G035829 | GACGGUUUUGUCCCUGUUGG | 49 |
| G035830 | CUUGAAAUGUAGACCCCCAG | 50 |
| G035831 | AAGAAGCACCAGGUACAGCG | 51 |
| G035832 | UUGCCACAACUACCAGGUGG | 52 |
| G035833 | UUCUUGAAAUGUAGACCCCC | 53 |
| G035834 | AGCACCAGGUACAGCGGGGG | 54 |
| G035835 | UUAUGAUGAACAGUGUCAUC | 55 |
| G035836 | CAUGUUAAAAUGAACACGGG | 56 |
| G035837 | GAAGAGACACUCCACCUGGA | 57 |
| G035838 | UCUGCUCUCCAGCACUCGGG | 58 |
| G035839 | GUCUGAGCAGUAGUCCAGGA | 59 |
| G035840 | AUCUGGCAUACAGAGCCCCA | 60 |
| G035841 | CACAACUACCAGGUGGGGGG | 61 |
| G035842 | AGAGUAACAGUGCAGUGUGC | 62 |
| G035843 | UUUAAGAGUCGAGAAAUUCU | 63 |
| G035844 | GUUUUGUCCCUGUUGGGGGU | 64 |
| G035845 | GUGGCUGGCCGUCCUGUGCA | 65 |
| G035846 | CAUCUGGCAUACAGAGCCCC | 66 |
| G035847 | CUGUGGCCAGUAGGUGACUU | 67 |
| G035848 | UCUUGCAUAUAACAUUAGGA | 68 |
| G035849 | AUGAAGGGGCACAAGUGGCA | 69 |
| G035850 | AAAAGUCAUUGGGAACCCCU | 70 |
| G035851 | GUGGAGGGAUUAAUGACAGA | 71 |
| G035852 | CUGCAGAAGCACCCCACAGA | 72 |
| G035853 | AGGCAAGGACAGGGGCCAUC | 73 |
| G035854 | UCUGGCAUACAGAGCCCCAG | 74 |
| G035855 | GAAGAAGUAGGCAAGGACAG | 75 |
| G035856 | AGAACCAUUCCUCUGGAUCG | 76 |
| G035857 | CAGCUAGCUGAGACCUAUAC | 77 |
| G035858 | GGUUGUCUGAGCAGUAGUCC | 78 |
| G035859 | GAAACCCCGAUCCAGAGGAA | 79 |
| G035860 | UCACAUAGCGAGGAAGCCCU | 80 |
| G035861 | CAGAGAACUCCAAUUGAGUC | 81 |
| G035862 | GGGACAAAACCGUCUCUGGU | 82 |
| G035863 | AUCACAUAGCGAGGAAGCCC | 83 |
| G035864 | AUAAAUGCCCUGUAUGGGAC | 84 |
| G035865 | AGACGGUUUUGUCCCUGUUG | 85 |
| G035866 | AAAUGCCCUGUAUGGGACAG | 86 |
| G035867 | UGAUUGUGAAUCUAGGCAUG | 87 |
| G035868 | GAGACGGUUUUGUCCCUGUU | 88 |
| G035869 | ACUGAGAAACCCCGAUCCAG | 89 |
| G035870 | CAUAGCGAGGAAGCCCUGGG | 90 |
| G035871 | ACAUUCUAGUUCCAGGCCUG | 91 |
| G035872 | ACUCGGGAGGCACAGAACCA | 92 |
| G035873 | CAUGCCUAGAUUCACAAUCA | 93 |
| G035874 | GGAACUAGAAUGUCCCUCCU | 94 |
| G035875 | CUCAGCUAGCUGAGGCCCCU | 95 |
| G035876 | AAGAGACACUCCACCUGGAU | 96 |
| G035877 | GAAAAGUCAUUGGGAACCCC | 97 |
| G035878 | GCCAGUAGGUGACUUGGGAC | 98 |
| G035879 | AUCAAGGCACCAGACUCAAU | 99 |
| G035880 | AUCAGGAUUGAUUAGCAGGC | 100 |
| G035881 | UGCCACAACUACCAGGUGGG | 101 |
| G035882 | GACACUCCACCUGGAUGGGA | 102 |
| G035883 | GCAGACCCCUGUCCCAUACA | 103 |
| G035884 | GGGAUCAGCUCUGACCCUGC | 104 |
| G035885 | UCAGCUAGCUGAGGCCCCUG | 105 |
| G035886 | CUGGGGCUCUGUAUGCCAGA | 106 |
| G035887 | GAGUAACAGUGCAGUGUGCU | 107 |
| G035888 | GUGCUUCUGCAGGGUUGCCA | 108 |
| G035889 | UCAGAGCUGAUCCCCUCUGU | 109 |
| G035890 | CGGGAGGCACAGAACCAAGG | 110 |
| G035891 | UGUCUCCCUGAAAAGUCAUU | 111 |
| G035892 | AAAGUCAUUGGGAACCCCUG | 112 |
| G035893 | GUGGUGGAUGCUCCUGCCCC | 113 |
| G035894 | GUGGAAUUCUGGCAUCUAGU | 114 |
| G035895 | CCUGUGGCCAGUAGGUGACU | 115 |
| G035896 | AUGGAUAGAAAAACGGUGAA | 116 |
| G035897 | CUCCACCUGGAUGGGAAGGU | 117 |
| G035898 | ACUCCACCUGGAUGGGAAGG | 118 |

TABLE 3
Human ASS1 Intron 2 sgRNA Sequences with scaffold A.

| Corr. to | | |
|---|---|---|
| Guide | SEQ | |
| RNA ID# | ID NO: | Full Sequence |
| G035811 | 252 | GAGUGCGCUUUCAGCAGCGCGUUUUAGAGCUAGAAAUAGCAAGUUAAAAU |
| | | AAGGCUAGUCCGUUAUCAACUUGAAAAAGUGGCACCGAGUCGGUGCUUUU |
| G035812 | 253 | CCUCUACACUCUAAUUUACGGUUUUAGAGCUAGAAAUAGCAAGUUAAAAUA |
| | | AGGCUAGUCCGUUAUCAACUUGAAAAAGUGGCACCGAGUCGGUGCUUUU |
| G035813 | 254 | CAGCGGGGGUGGCCCCGUAUGUUUUAGAGCUAGAAAUAGCAAGUUAAAAU |
| | | AAGGCUAGUCCGUUAUCAACUUGAAAAAGUGGCACCGAGUCGGUGCUUUU |
| G035814 | 255 | AGCUAGCUGAGACCUAUACGGUUUUAGAGCUAGAAAUAGCAAGUUAAAAU |
| | | AAGGCUAGUCCGUUAUCAACUUGAAAAAGUGGCACCGAGUCGGUGCUUUU |
| G035815 | 256 | CCACGUAAAUUAGAGUGUAGGUUUUAGAGCUAGAAAUAGCAAGUUAAAAU |
| | | AAGGCUAGUCCGUUAUCAACUUGAAAAAGUGGCACCGAGUCGGUGCUUUU |
| G035816 | 257 | CACUCGAAUCUUCAACACUCGUUUUAGAGCUAGAAAUAGCAAGUUAAAAUA |
| | | AGGCUAGUCCGUUAUCAACUUGAAAAAGUGGCACCGAGUCGGUGCUUUU |
| G035817 | 258 | ACUCGAAUCUUCAACACUCAGUUUUAGAGCUAGAAAUAGCAAGUUAAAAUA |
| | | AGGCUAGUCCGUUAUCAACUUGAAAAAGUGGCACCGAGUCGGUGCUUUU |
| G035818 | 259 | GUUUCACAGAACGGUGCAUCGUUUUAGAGCUAGAAAUAGCAAGUUAAAAU |
| | | AAGGCUAGUCCGUUAUCAACUUGAAAAAGUGGCACCGAGUCGGUGCUUUU |
| G035819 | 260 | GGUUGCCACAACUACCAGGUGUUUUAGAGCUAGAAAUAGCAAGUUAAAAU |
| | | AAGGCUAGUCCGUUAUCAACUUGAAAAAGUGGCACCGAGUCGGUGCUUUU |
| G035820 | 261 | UGGCCUUGUUCCCGUGUGGCGUUUUAGAGCUAGAAAUAGCAAGUUAAAAU |
| | | AAGGCUAGUCCGUUAUCAACUUGAAAAAGUGGCACCGAGUCGGUGCUUUU |
| G035821 | 262 | CGGCCAGCCACACGGGAACAGUUUUAGAGCUAGAAAUAGCAAGUUAAAAUA |
| | | AGGCUAGUCCGUUAUCAACUUGAAAAAGUGGCACCGAGUCGGUGCUUUU |
| G035822 | 263 | AAACAAGGCAUCACAUAGCGGUUUUAGAGCUAGAAAUAGCAAGUUAAAAU |
| | | AAGGCUAGUCCGUUAUCAACUUGAAAAAGUGGCACCGAGUCGGUGCUUUU |
| G035823 | 264 | GUAUAGGUCUCAGCUAGCUGGUUUUAGAGCUAGAAAUAGCAAGUUAAAAU |
| | | AAGGCUAGUCCGUUAUCAACUUGAAAAAGUGGCACCGAGUCGGUGCUUUU |
| G035824 | 265 | GGUGCCUUGAUUGUGAAUCUGUUUUAGAGCUAGAAAUAGCAAGUUAAAAU |
| | | AAGGCUAGUCCGUUAUCAACUUGAAAAAGUGGCACCGAGUCGGUGCUUUU |
| G035825 | 266 | GUGCAUCAGGAUUGAUUAGCGUUUUAGAGCUAGAAAUAGCAAGUUAAAAU |
| | | AAGGCUAGUCCGUUAUCAACUUGAAAAAGUGGCACCGAGUCGGUGCUUUU |
| G035826 | 267 | GGGGUUCCCAAUGACUUUUCGUUUUAGAGCUAGAAAUAGCAAGUUAAAAU |
| | | AAGGCUAGUCCGUUAUCAACUUGAAAAAGUGGCACCGAGUCGGUGCUUUU |
| G035827 | 268 | UCAGCUAGCUGAGACCUAUAGUUUUAGAGCUAGAAAUAGCAAGUUAAAAU |
| | | AAGGCUAGUCCGUUAUCAACUUGAAAAAGUGGCACCGAGUCGGUGCUUUU |
| G035828 | 269 | GAUGCACCGUUCUGUGAAACGUUUUAGAGCUAGAAAUAGCAAGUUAAAAU |
| | | AAGGCUAGUCCGUUAUCAACUUGAAAAAGUGGCACCGAGUCGGUGCUUUU |
| G035829 | 270 | GACGGUUUUGUCCCUGUUGGGUUUUAGAGCUAGAAAUAGCAAGUUAAAAU |
| | | AAGGCUAGUCCGUUAUCAACUUGAAAAAGUGGCACCGAGUCGGUGCUUUU |
| G035830 | 271 | CUUGAAAUGUAGACCCCCAGGUUUUAGAGCUAGAAAUAGCAAGUUAAAAU |
| | | AAGGCUAGUCCGUUAUCAACUUGAAAAAGUGGCACCGAGUCGGUGCUUUU |
| G035831 | 272 | AAGAAGCACCAGGUACAGCGGUUUUAGAGCUAGAAAUAGCAAGUUAAAAU |
| | | AAGGCUAGUCCGUUAUCAACUUGAAAAAGUGGCACCGAGUCGGUGCUUUU |
| G035832 | 273 | UUGCCACAACUACCAGGUGGGUUUUAGAGCUAGAAAUAGCAAGUUAAAAU |
| | | AAGGCUAGUCCGUUAUCAACUUGAAAAAGUGGCACCGAGUCGGUGCUUUU |
| G035833 | 274 | UUCUUGAAAUGUAGACCCCCGUUUUAGAGCUAGAAAUAGCAAGUUAAAAU |
| | | AAGGCUAGUCCGUUAUCAACUUGAAAAAGUGGCACCGAGUCGGUGCUUUU |
| G035834 | 275 | AGCACCAGGUACAGCGGGGGGUUUUAGAGCUAGAAAUAGCAAGUUAAAAU |
| | | AAGGCUAGUCCGUUAUCAACUUGAAAAAGUGGCACCGAGUCGGUGCUUUU |
| G035835 | 276 | UUAUGAUGAACAGUGUCAUCGUUUUAGAGCUAGAAAUAGCAAGUUAAAAU |
| | | AAGGCUAGUCCGUUAUCAACUUGAAAAAGUGGCACCGAGUCGGUGCUUUU |
| G035836 | 277 | CAUGUUAAAAUGAACACGGGGUUUUAGAGCUAGAAAUAGCAAGUUAAAAU |
| | | AAGGCUAGUCCGUUAUCAACUUGAAAAAGUGGCACCGAGUCGGUGCUUUU |
| G035837 | 278 | GAAGAGACACUCCACCUGGAGUUUUAGAGCUAGAAAUAGCAAGUUAAAAU |
| | | AAGGCUAGUCCGUUAUCAACUUGAAAAAGUGGCACCGAGUCGGUGCUUUU |
| G035838 | 279 | UCUGCUCUCCAGCACUCGGGGUUUUAGAGCUAGAAAUAGCAAGUUAAAAUA |
| | | AGGCUAGUCCGUUAUCAACUUGAAAAAGUGGCACCGAGUCGGUGCUUUU |
| G035839 | 280 | GUCUGAGCAGUAGUCCAGGAGUUUUAGAGCUAGAAAUAGCAAGUUAAAAU |
| | | AAGGCUAGUCCGUUAUCAACUUGAAAAAGUGGCACCGAGUCGGUGCUUUU |
| G035840 | 281 | AUCUGGCAUACAGAGCCCCAGUUUUAGAGCUAGAAAUAGCAAGUUAAAAUA |
| | | AGGCUAGUCCGUUAUCAACUUGAAAAAGUGGCACCGAGUCGGUGCUUUU |
| G035841 | 282 | CACAACUACCAGGUGGGGGGGUUUUAGAGCUAGAAAUAGCAAGUUAAAAU |
| | | AAGGCUAGUCCGUUAUCAACUUGAAAAAGUGGCACCGAGUCGGUGCUUUU |
| G035842 | 283 | AGAGUAACAGUGCAGUGUGCGUUUUAGAGCUAGAAAUAGCAAGUUAAAAU |
| | | AAGGCUAGUCCGUUAUCAACUUGAAAAAGUGGCACCGAGUCGGUGCUUUU |
| G035843 | 284 | UUUAAGAGUCGAGAAAUUCUGUUUUAGAGCUAGAAAUAGCAAGUUAAAAU |
| | | AAGGCUAGUCCGUUAUCAACUUGAAAAAGUGGCACCGAGUCGGUGCUUUU |
| G035844 | 285 | GUUUUGUCCCUGUUGGGGGUGUUUUAGAGCUAGAAAUAGCAAGUUAAAAU |
| | | AAGGCUAGUCCGUUAUCAACUUGAAAAAGUGGCACCGAGUCGGUGCUUUU |
| G035845 | 286 | GUGGCUGGCCGUCCUGUGCAGUUUUAGAGCUAGAAAUAGCAAGUUAAAAU |
| | | AAGGCUAGUCCGUUAUCAACUUGAAAAAGUGGCACCGAGUCGGUGCUUUU |
| G035846 | 287 | CAUCUGGCAUACAGAGCCCCGUUUUAGAGCUAGAAAUAGCAAGUUAAAAUA |
| | | AGGCUAGUCCGUUAUCAACUUGAAAAAGUGGCACCGAGUCGGUGCUUUU |
| G035847 | 288 | CUGUGGCCAGUAGGUGACUUGUUUUAGAGCUAGAAAUAGCAAGUUAAAAU |
| | | AAGGCUAGUCCGUUAUCAACUUGAAAAAGUGGCACCGAGUCGGUGCUUUU |
| G035848 | 289 | UCUUGCAUAUAACAUUAGGAGUUUUAGAGCUAGAAAUAGCAAGUUAAAAU |
| | | AAGGCUAGUCCGUUAUCAACUUGAAAAAGUGGCACCGAGUCGGUGCUUUU |
| G035849 | 290 | AUGAAGGGGCACAAGUGGCAGUUUUAGAGCUAGAAAUAGCAAGUUAAAAU |
| | | AAGGCUAGUCCGUUAUCAACUUGAAAAAGUGGCACCGAGUCGGUGCUUUU |
| G035850 | 291 | AAAAGUCAUUGGGAACCCCUGUUUUAGAGCUAGAAAUAGCAAGUUAAAAU |
| | | AAGGCUAGUCCGUUAUCAACUUGAAAAAGUGGCACCGAGUCGGUGCUUUU |
| G035851 | 292 | GUGGAGGGAUUAAUGACAGAGUUUUAGAGCUAGAAAUAGCAAGUUAAAAU |
| | | AAGGCUAGUCCGUUAUCAACUUGAAAAAGUGGCACCGAGUCGGUGCUUUU |
| G035852 | 293 | CUGCAGAAGCACCCCACAGAGUUUUAGAGCUAGAAAUAGCAAGUUAAAAUA |
| | | AGGCUAGUCCGUUAUCAACUUGAAAAAGUGGCACCGAGUCGGUGCUUUU |
| G035853 | 294 | AGGCAAGGACAGGGGCCAUCGUUUUAGAGCUAGAAAUAGCAAGUUAAAAU |
| | | AAGGCUAGUCCGUUAUCAACUUGAAAAAGUGGCACCGAGUCGGUGCUUUU |
| G035854 | 295 | UCUGGCAUACAGAGCCCCAGGUUUUAGAGCUAGAAAUAGCAAGUUAAAAUA |
| | | AGGCUAGUCCGUUAUCAACUUGAAAAAGUGGCACCGAGUCGGUGCUUUU |
| G035855 | 296 | GAAGAAGUAGGCAAGGACAGGUUUUAGAGCUAGAAAUAGCAAGUUAAAAU |
| | | AAGGCUAGUCCGUUAUCAACUUGAAAAAGUGGCACCGAGUCGGUGCUUUU |
| G035856 | 297 | AGAACCAUUCCUCUGGAUCGGUUUUAGAGCUAGAAAUAGCAAGUUAAAAU |
| | | AAGGCUAGUCCGUUAUCAACUUGAAAAAGUGGCACCGAGUCGGUGCUUUU |
| G035857 | 298 | CAGCUAGCUGAGACCUAUACGUUUUAGAGCUAGAAAUAGCAAGUUAAAAU |
| | | AAGGCUAGUCCGUUAUCAACUUGAAAAAGUGGCACCGAGUCGGUGCUUUU |
| G035858 | 299 | GGUUGUCUGAGCAGUAGUCCGUUUUAGAGCUAGAAAUAGCAAGUUAAAAU |
| | | AAGGCUAGUCCGUUAUCAACUUGAAAAAGUGGCACCGAGUCGGUGCUUUU |
| G035859 | 300 | GAAACCCCGAUCCAGAGGAAGUUUUAGAGCUAGAAAUAGCAAGUUAAAAU |
| | | AAGGCUAGUCCGUUAUCAACUUGAAAAAGUGGCACCGAGUCGGUGCUUUU |
| G035860 | 301 | UCACAUAGCGAGGAAGCCCUGUUUUAGAGCUAGAAAUAGCAAGUUAAAAU |
| | | AAGGCUAGUCCGUUAUCAACUUGAAAAAGUGGCACCGAGUCGGUGCUUUU |
| G035861 | 302 | CAGAGAACUCCAAUUGAGUCGUUUUAGAGCUAGAAAUAGCAAGUUAAAAU |
| | | AAGGCUAGUCCGUUAUCAACUUGAAAAAGUGGCACCGAGUCGGUGCUUUU |
| G035862 | 303 | GGGACAAAACCGUCUCUGGUGUUUUAGAGCUAGAAAUAGCAAGUUAAAAU |
| | | AAGGCUAGUCCGUUAUCAACUUGAAAAAGUGGCACCGAGUCGGUGCUUUU |
| G035863 | 304 | AUCACAUAGCGAGGAAGCCCGUUUUAGAGCUAGAAAUAGCAAGUUAAAAU |
| | | AAGGCUAGUCCGUUAUCAACUUGAAAAAGUGGCACCGAGUCGGUGCUUUU |
| G035864 | 305 | AUAAAUGCCCUGUAUGGGACGUUUUAGAGCUAGAAAUAGCAAGUUAAAAU |
| | | AAGGCUAGUCCGUUAUCAACUUGAAAAAGUGGCACCGAGUCGGUGCUUUU |
| G035865 | 306 | AGACGGUUUUGUCCCUGUUGGUUUUAGAGCUAGAAAUAGCAAGUUAAAAU |
| | | AAGGCUAGUCCGUUAUCAACUUGAAAAAGUGGCACCGAGUCGGUGCUUUU |
| G035866 | 307 | AAAUGCCCUGUAUGGGACAGGUUUUAGAGCUAGAAAUAGCAAGUUAAAAU |
| | | AAGGCUAGUCCGUUAUCAACUUGAAAAAGUGGCACCGAGUCGGUGCUUUU |
| G035867 | 308 | UGAUUGUGAAUCUAGGCAUGGUUUUAGAGCUAGAAAUAGCAAGUUAAAAU |
| | | AAGGCUAGUCCGUUAUCAACUUGAAAAAGUGGCACCGAGUCGGUGCUUUU |
| G035868 | 309 | GAGACGGUUUUGUCCCUGUUGUUUUAGAGCUAGAAAUAGCAAGUUAAAAU |
| | | AAGGCUAGUCCGUUAUCAACUUGAAAAAGUGGCACCGAGUCGGUGCUUUU |
| G035869 | 310 | ACUGAGAAACCCCGAUCCAGGUUUUAGAGCUAGAAAUAGCAAGUUAAAAUA |
| | | AGGCUAGUCCGUUAUCAACUUGAAAAAGUGGCACCGAGUCGGUGCUUUU |
| G035870 | 311 | CAUAGCGAGGAAGCCCUGGGGUUUUAGAGCUAGAAAUAGCAAGUUAAAAU |
| | | AAGGCUAGUCCGUUAUCAACUUGAAAAAGUGGCACCGAGUCGGUGCUUUU |
| G035871 | 312 | ACAUUCUAGUUCCAGGCCUGGUUUUAGAGCUAGAAAUAGCAAGUUAAAAU |
| | | AAGGCUAGUCCGUUAUCAACUUGAAAAAGUGGCACCGAGUCGGUGCUUUU |
| G035872 | 313 | ACUCGGGAGGCACAGAACCAGUUUUAGAGCUAGAAAUAGCAAGUUAAAAU |
| | | AAGGCUAGUCCGUUAUCAACUUGAAAAAGUGGCACCGAGUCGGUGCUUUU |
| G035873 | 314 | CAUGCCUAGAUUCACAAUCAGUUUUAGAGCUAGAAAUAGCAAGUUAAAAU |
| | | AAGGCUAGUCCGUUAUCAACUUGAAAAAGUGGCACCGAGUCGGUGCUUUU |
| G035874 | 315 | GGAACUAGAAUGUCCCUCCUGUUUUAGAGCUAGAAAUAGCAAGUUAAAAU |
| | | AAGGCUAGUCCGUUAUCAACUUGAAAAAGUGGCACCGAGUCGGUGCUUUU |
| G035875 | 316 | CUCAGCUAGCUGAGGCCCCUGUUUUAGAGCUAGAAAUAGCAAGUUAAAAUA |
| | | AGGCUAGUCCGUUAUCAACUUGAAAAAGUGGCACCGAGUCGGUGCUUUU |
| G035876 | 317 | AAGAGACACUCCACCUGGAUGUUUUAGAGCUAGAAAUAGCAAGUUAAAAU |
| | | AAGGCUAGUCCGUUAUCAACUUGAAAAAGUGGCACCGAGUCGGUGCUUUU |
| G035877 | 318 | GAAAAGUCAUUGGGAACCCCGUUUUAGAGCUAGAAAUAGCAAGUUAAAAU |
| | | AAGGCUAGUCCGUUAUCAACUUGAAAAAGUGGCACCGAGUCGGUGCUUUU |
| G035878 | 319 | GCCAGUAGGUGACUUGGGACGUUUUAGAGCUAGAAAUAGCAAGUUAAAAU |
| | | AAGGCUAGUCCGUUAUCAACUUGAAAAAGUGGCACCGAGUCGGUGCUUUU |
| G035879 | 320 | AUCAAGGCACCAGACUCAAUGUUUUAGAGCUAGAAAUAGCAAGUUAAAAU |
| | | AAGGCUAGUCCGUUAUCAACUUGAAAAAGUGGCACCGAGUCGGUGCUUUU |
| G035880 | 321 | AUCAGGAUUGAUUAGCAGGCGUUUUAGAGCUAGAAAUAGCAAGUUAAAAU |
| | | AAGGCUAGUCCGUUAUCAACUUGAAAAAGUGGCACCGAGUCGGUGCUUUU |
| G035881 | 322 | UGCCACAACUACCAGGUGGGGUUUUAGAGCUAGAAAUAGCAAGUUAAAAU |
| | | AAGGCUAGUCCGUUAUCAACUUGAAAAAGUGGCACCGAGUCGGUGCUUUU |
| G035882 | 323 | GACACUCCACCUGGAUGGGAGUUUUAGAGCUAGAAAUAGCAAGUUAAAAU |
| | | AAGGCUAGUCCGUUAUCAACUUGAAAAAGUGGCACCGAGUCGGUGCUUUU |
| G035883 | 324 | GCAGACCCCUGUCCCAUACAGUUUUAGAGCUAGAAAUAGCAAGUUAAAAUA |
| | | AGGCUAGUCCGUUAUCAACUUGAAAAAGUGGCACCGAGUCGGUGCUUUU |
| G035884 | 325 | GGGAUCAGCUCUGACCCUGCGUUUUAGAGCUAGAAAUAGCAAGUUAAAAUA |
| | | AGGCUAGUCCGUUAUCAACUUGAAAAAGUGGCACCGAGUCGGUGCUUUU |
| G035885 | 326 | UCAGCUAGCUGAGGCCCCUGGUUUUAGAGCUAGAAAUAGCAAGUUAAAAUA |
| | | AGGCUAGUCCGUUAUCAACUUGAAAAAGUGGCACCGAGUCGGUGCUUUU |
| G035886 | 327 | CUGGGGCUCUGUAUGCCAGAGUUUUAGAGCUAGAAAUAGCAAGUUAAAAU |
| | | AAGGCUAGUCCGUUAUCAACUUGAAAAAGUGGCACCGAGUCGGUGCUUUU |
| G035887 | 328 | GAGUAACAGUGCAGUGUGCUGUUUUAGAGCUAGAAAUAGCAAGUUAAAAU |
| | | AAGGCUAGUCCGUUAUCAACUUGAAAAAGUGGCACCGAGUCGGUGCUUUU |
| G035888 | 329 | GUGCUUCUGCAGGGUUGCCAGUUUUAGAGCUAGAAAUAGCAAGUUAAAAU |
| | | AAGGCUAGUCCGUUAUCAACUUGAAAAAGUGGCACCGAGUCGGUGCUUUU |
| G035889 | 330 | UCAGAGCUGAUCCCCUCUGUGUUUUAGAGCUAGAAAUAGCAAGUUAAAAUA |
| | | AGGCUAGUCCGUUAUCAACUUGAAAAAGUGGCACCGAGUCGGUGCUUUU |
| G035890 | 331 | CGGGAGGCACAGAACCAAGGGUUUUAGAGCUAGAAAUAGCAAGUUAAAAU |
| | | AAGGCUAGUCCGUUAUCAACUUGAAAAAGUGGCACCGAGUCGGUGCUUUU |
| G035891 | 332 | UGUCUCCCUGAAAAGUCAUUGUUUUAGAGCUAGAAAUAGCAAGUUAAAAU |
| | | AAGGCUAGUCCGUUAUCAACUUGAAAAAGUGGCACCGAGUCGGUGCUUUU |
| G035892 | 333 | AAAGUCAUUGGGAACCCCUGGUUUUAGAGCUAGAAAUAGCAAGUUAAAAU |
| | | AAGGCUAGUCCGUUAUCAACUUGAAAAAGUGGCACCGAGUCGGUGCUUUU |
| G035893 | 334 | GUGGUGGAUGCUCCUGCCCCGUUUUAGAGCUAGAAAUAGCAAGUUAAAAUA |
| | | AGGCUAGUCCGUUAUCAACUUGAAAAAGUGGCACCGAGUCGGUGCUUUU |
| G035894 | 335 | GUGGAAUUCUGGCAUCUAGUGUUUUAGAGCUAGAAAUAGCAAGUUAAAAU |
| | | AAGGCUAGUCCGUUAUCAACUUGAAAAAGUGGCACCGAGUCGGUGCUUUU |
| G035895 | 336 | CCUGUGGCCAGUAGGUGACUGUUUUAGAGCUAGAAAUAGCAAGUUAAAAU |
| | | AAGGCUAGUCCGUUAUCAACUUGAAAAAGUGGCACCGAGUCGGUGCUUUU |
| G035896 | 337 | AUGGAUAGAAAAACGGUGAAGUUUUAGAGCUAGAAAUAGCAAGUUAAAAU |
| | | AAGGCUAGUCCGUUAUCAACUUGAAAAAGUGGCACCGAGUCGGUGCUUUU |
| G035897 | 338 | CUCCACCUGGAUGGGAAGGUGUUUUAGAGCUAGAAAUAGCAAGUUAAAAU |
| | | AAGGCUAGUCCGUUAUCAACUUGAAAAAGUGGCACCGAGUCGGUGCUUUU |
| G035898 | 339 | ACUCCACCUGGAUGGGAAGGGUUUUAGAGCUAGAAAUAGCAAGUUAAAAU |
| | | AAGGCUAGUCCGUUAUCAACUUGAAAAAGUGGCACCGAGUCGGUGCUUUU |

TABLE 4
Human ASS1 Intron 2 sgRNA Sequences with scaffold B.

| Guide | SEQ | |
|---|---|---|
| RNA ID# | ID NO: | Full Sequence |
| G035811 | 340 | GAGUGCGCUUUCAGCAGCGCGUUUUAGAGCUAGAAAUAGCAAGUUAAAAU |
| | | AAGGCUAGUCCGUUAUCACGAAAGGGCACCGAGUCGGUGCU |
| G035812 | 341 | CCUCUACACUCUAAUUUACGGUUUUAGAGCUAGAAAUAGCAAGUUAAAAUA |
| | | AGGCUAGUCCGUUAUCACGAAAGGGCACCGAGUCGGUGCU |
| G035813 | 342 | CAGCGGGGGUGGCCCCGUAUGUUUUAGAGCUAGAAAUAGCAAGUUAAAAU |
| | | AAGGCUAGUCCGUUAUCACGAAAGGGCACCGAGUCGGUGCU |
| G035814 | 343 | AGCUAGCUGAGACCUAUACGGUUUUAGAGCUAGAAAUAGCAAGUUAAAAU |
| | | AAGGCUAGUCCGUUAUCACGAAAGGGCACCGAGUCGGUGCU |
| G035815 | 344 | CCACGUAAAUUAGAGUGUAGGUUUUAGAGCUAGAAAUAGCAAGUUAAAAU |
| | | AAGGCUAGUCCGUUAUCACGAAAGGGCACCGAGUCGGUGCU |
| G035816 | 345 | CACUCGAAUCUUCAACACUCGUUUUAGAGCUAGAAAUAGCAAGUUAAAAUA |
| | | AGGCUAGUCCGUUAUCACGAAAGGGCACCGAGUCGGUGCU |
| G035817 | 346 | ACUCGAAUCUUCAACACUCAGUUUUAGAGCUAGAAAUAGCAAGUUAAAAUA |
| | | AGGCUAGUCCGUUAUCACGAAAGGGCACCGAGUCGGUGCU |
| G035818 | 347 | GUUUCACAGAACGGUGCAUCGUUUUAGAGCUAGAAAUAGCAAGUUAAAAU |
| | | AAGGCUAGUCCGUUAUCACGAAAGGGCACCGAGUCGGUGCU |
| G035819 | 348 | GGUUGCCACAACUACCAGGUGUUUUAGAGCUAGAAAUAGCAAGUUAAAAU |
| | | AAGGCUAGUCCGUUAUCACGAAAGGGCACCGAGUCGGUGCU |
| G035820 | 349 | UGGCCUUGUUCCCGUGUGGCGUUUUAGAGCUAGAAAUAGCAAGUUAAAAU |
| | | AAGGCUAGUCCGUUAUCACGAAAGGGCACCGAGUCGGUGCU |
| G035821 | 350 | CGGCCAGCCACACGGGAACAGUUUUAGAGCUAGAAAUAGCAAGUUAAAAUA |
| | | AGGCUAGUCCGUUAUCACGAAAGGGCACCGAGUCGGUGCU |
| G035822 | 351 | AAACAAGGCAUCACAUAGCGGUUUUAGAGCUAGAAAUAGCAAGUUAAAAU |
| | | AAGGCUAGUCCGUUAUCACGAAAGGGCACCGAGUCGGUGCU |
| G035823 | 352 | GUAUAGGUCUCAGCUAGCUGGUUUUAGAGCUAGAAAUAGCAAGUUAAAAU |
| | | AAGGCUAGUCCGUUAUCACGAAAGGGCACCGAGUCGGUGCU |
| G035824 | 353 | GGUGCCUUGAUUGUGAAUCUGUUUUAGAGCUAGAAAUAGCAAGUUAAAAU |
| | | AAGGCUAGUCCGUUAUCACGAAAGGGCACCGAGUCGGUGCU |
| G035825 | 354 | GUGCAUCAGGAUUGAUUAGCGUUUUAGAGCUAGAAAUAGCAAGUUAAAAU |
| | | AAGGCUAGUCCGUUAUCACGAAAGGGCACCGAGUCGGUGCU |
| G035826 | 355 | GGGGUUCCCAAUGACUUUUCGUUUUAGAGCUAGAAAUAGCAAGUUAAAAU |
| | | AAGGCUAGUCCGUUAUCACGAAAGGGCACCGAGUCGGUGCU |
| G035827 | 356 | UCAGCUAGCUGAGACCUAUAGUUUUAGAGCUAGAAAUAGCAAGUUAAAAU |
| | | AAGGCUAGUCCGUUAUCACGAAAGGGCACCGAGUCGGUGCU |
| G035828 | 357 | GAUGCACCGUUCUGUGAAACGUUUUAGAGCUAGAAAUAGCAAGUUAAAAU |
| | | AAGGCUAGUCCGUUAUCACGAAAGGGCACCGAGUCGGUGCU |
| G035829 | 358 | GACGGUUUUGUCCCUGUUGGGUUUUAGAGCUAGAAAUAGCAAGUUAAAAU |
| | | AAGGCUAGUCCGUUAUCACGAAAGGGCACCGAGUCGGUGCU |
| G035830 | 359 | CUUGAAAUGUAGACCCCCAGGUUUUAGAGCUAGAAAUAGCAAGUUAAAAU |
| | | AAGGCUAGUCCGUUAUCACGAAAGGGCACCGAGUCGGUGCU |
| G035831 | 360 | AAGAAGCACCAGGUACAGCGGUUUUAGAGCUAGAAAUAGCAAGUUAAAAU |
| | | AAGGCUAGUCCGUUAUCACGAAAGGGCACCGAGUCGGUGCU |
| G035832 | 361 | UUGCCACAACUACCAGGUGGGUUUUAGAGCUAGAAAUAGCAAGUUAAAAU |
| | | AAGGCUAGUCCGUUAUCACGAAAGGGCACCGAGUCGGUGCU |
| G035833 | 362 | UUCUUGAAAUGUAGACCCCCGUUUUAGAGCUAGAAAUAGCAAGUUAAAAU |
| | | AAGGCUAGUCCGUUAUCACGAAAGGGCACCGAGUCGGUGCU |
| G035834 | 363 | AGCACCAGGUACAGCGGGGGGUUUUAGAGCUAGAAAUAGCAAGUUAAAAU |
| | | AAGGCUAGUCCGUUAUCACGAAAGGGCACCGAGUCGGUGCU |
| G035835 | 364 | UUAUGAUGAACAGUGUCAUCGUUUUAGAGCUAGAAAUAGCAAGUUAAAAU |
| | | AAGGCUAGUCCGUUAUCACGAAAGGGCACCGAGUCGGUGCU |
| G035836 | 365 | CAUGUUAAAAUGAACACGGGGUUUUAGAGCUAGAAAUAGCAAGUUAAAAU |
| | | AAGGCUAGUCCGUUAUCACGAAAGGGCACCGAGUCGGUGCU |
| G035837 | 366 | GAAGAGACACUCCACCUGGAGUUUUAGAGCUAGAAAUAGCAAGUUAAAAU |
| | | AAGGCUAGUCCGUUAUCACGAAAGGGCACCGAGUCGGUGCU |
| G035838 | 367 | UCUGCUCUCCAGCACUCGGGGUUUUAGAGCUAGAAAUAGCAAGUUAAAAUA |
| | | AGGCUAGUCCGUUAUCACGAAAGGGCACCGAGUCGGUGCU |
| G035839 | 368 | GUCUGAGCAGUAGUCCAGGAGUUUUAGAGCUAGAAAUAGCAAGUUAAAAU |
| | | AAGGCUAGUCCGUUAUCACGAAAGGGCACCGAGUCGGUGCU |
| G035840 | 369 | AUCUGGCAUACAGAGCCCCAGUUUUAGAGCUAGAAAUAGCAAGUUAAAAUA |
| | | AGGCUAGUCCGUUAUCACGAAAGGGCACCGAGUCGGUGCU |
| G035841 | 370 | CACAACUACCAGGUGGGGGGGUUUUAGAGCUAGAAAUAGCAAGUUAAAAU |
| | | AAGGCUAGUCCGUUAUCACGAAAGGGCACCGAGUCGGUGCU |
| G035842 | 371 | AGAGUAACAGUGCAGUGUGCGUUUUAGAGCUAGAAAUAGCAAGUUAAAAU |
| | | AAGGCUAGUCCGUUAUCACGAAAGGGCACCGAGUCGGUGCU |
| G035843 | 372 | UUUAAGAGUCGAGAAAUUCUGUUUUAGAGCUAGAAAUAGCAAGUUAAAAU |
| | | AAGGCUAGUCCGUUAUCACGAAAGGGCACCGAGUCGGUGCU |
| G035844 | 373 | GUUUUGUCCCUGUUGGGGGUGUUUUAGAGCUAGAAAUAGCAAGUUAAAAU |
| | | AAGGCUAGUCCGUUAUCACGAAAGGGCACCGAGUCGGUGCU |
| G035845 | 374 | GUGGCUGGCCGUCCUGUGCAGUUUUAGAGCUAGAAAUAGCAAGUUAAAAU |
| | | AAGGCUAGUCCGUUAUCACGAAAGGGCACCGAGUCGGUGCU |
| G035846 | 375 | CAUCUGGCAUACAGAGCCCCGUUUUAGAGCUAGAAAUAGCAAGUUAAAAUA |
| | | AGGCUAGUCCGUUAUCACGAAAGGGCACCGAGUCGGUGCU |
| G035847 | 376 | CUGUGGCCAGUAGGUGACUUGUUUUAGAGCUAGAAAUAGCAAGUUAAAAU |
| | | AAGGCUAGUCCGUUAUCACGAAAGGGCACCGAGUCGGUGCU |
| G035848 | 377 | UCUUGCAUAUAACAUUAGGAGUUUUAGAGCUAGAAAUAGCAAGUUAAAAU |
| | | AAGGCUAGUCCGUUAUCACGAAAGGGCACCGAGUCGGUGCU |
| G035849 | 378 | AUGAAGGGGCACAAGUGGCAGUUUUAGAGCUAGAAAUAGCAAGUUAAAAU |
| | | AAGGCUAGUCCGUUAUCACGAAAGGGCACCGAGUCGGUGCU |
| G035850 | 379 | AAAAGUCAUUGGGAACCCCUGUUUUAGAGCUAGAAAUAGCAAGUUAAAAU |
| | | AAGGCUAGUCCGUUAUCACGAAAGGGCACCGAGUCGGUGCU |
| G035851 | 380 | GUGGAGGGAUUAAUGACAGAGUUUUAGAGCUAGAAAUAGCAAGUUAAAAU |
| | | AAGGCUAGUCCGUUAUCACGAAAGGGCACCGAGUCGGUGCU |
| G035852 | 381 | CUGCAGAAGCACCCCACAGAGUUUUAGAGCUAGAAAUAGCAAGUUAAAAUA |
| | | AGGCUAGUCCGUUAUCACGAAAGGGCACCGAGUCGGUGCU |
| G035853 | 382 | AGGCAAGGACAGGGGCCAUCGUUUUAGAGCUAGAAAUAGCAAGUUAAAAU |
| | | AAGGCUAGUCCGUUAUCACGAAAGGGCACCGAGUCGGUGCU |
| G035854 | 383 | UCUGGCAUACAGAGCCCCAGGUUUUAGAGCUAGAAAUAGCAAGUUAAAAUA |
| | | AGGCUAGUCCGUUAUCACGAAAGGGCACCGAGUCGGUGCU |
| G035855 | 384 | GAAGAAGUAGGCAAGGACAGGUUUUAGAGCUAGAAAUAGCAAGUUAAAAU |
| | | AAGGCUAGUCCGUUAUCACGAAAGGGCACCGAGUCGGUGCU |
| G035856 | 385 | AGAACCAUUCCUCUGGAUCGGUUUUAGAGCUAGAAAUAGCAAGUUAAAAU |
| | | AAGGCUAGUCCGUUAUCACGAAAGGGCACCGAGUCGGUGCU |
| G035857 | 386 | CAGCUAGCUGAGACCUAUACGUUUUAGAGCUAGAAAUAGCAAGUUAAAAU |
| | | AAGGCUAGUCCGUUAUCACGAAAGGGCACCGAGUCGGUGCU |
| G035858 | 387 | GGUUGUCUGAGCAGUAGUCCGUUUUAGAGCUAGAAAUAGCAAGUUAAAAU |
| | | AAGGCUAGUCCGUUAUCACGAAAGGGCACCGAGUCGGUGCU |
| G035859 | 388 | GAAACCCCGAUCCAGAGGAAGUUUUAGAGCUAGAAAUAGCAAGUUAAAAU |
| | | AAGGCUAGUCCGUUAUCACGAAAGGGCACCGAGUCGGUGCU |
| G035860 | 389 | UCACAUAGCGAGGAAGCCCUGUUUUAGAGCUAGAAAUAGCAAGUUAAAAU |
| | | AAGGCUAGUCCGUUAUCACGAAAGGGCACCGAGUCGGUGCU |
| G035861 | 390 | CAGAGAACUCCAAUUGAGUCGUUUUAGAGCUAGAAAUAGCAAGUUAAAAU |
| | | AAGGCUAGUCCGUUAUCACGAAAGGGCACCGAGUCGGUGCU |
| G035862 | 391 | GGGACAAAACCGUCUCUGGUGUUUUAGAGCUAGAAAUAGCAAGUUAAAAU |
| | | AAGGCUAGUCCGUUAUCACGAAAGGGCACCGAGUCGGUGCU |
| G035863 | 392 | AUCACAUAGCGAGGAAGCCCGUUUUAGAGCUAGAAAUAGCAAGUUAAAAU |
| | | AAGGCUAGUCCGUUAUCACGAAAGGGCACCGAGUCGGUGCU |
| G035864 | 393 | AUAAAUGCCCUGUAUGGGACGUUUUAGAGCUAGAAAUAGCAAGUUAAAAU |
| | | AAGGCUAGUCCGUUAUCACGAAAGGGCACCGAGUCGGUGCU |
| G035865 | 394 | AGACGGUUUUGUCCCUGUUGGUUUUAGAGCUAGAAAUAGCAAGUUAAAAU |
| | | AAGGCUAGUCCGUUAUCACGAAAGGGCACCGAGUCGGUGCU |
| G035866 | 395 | AAAUGCCCUGUAUGGGACAGGUUUUAGAGCUAGAAAUAGCAAGUUAAAAU |
| | | AAGGCUAGUCCGUUAUCACGAAAGGGCACCGAGUCGGUGCU |
| G035867 | 396 | UGAUUGUGAAUCUAGGCAUGGUUUUAGAGCUAGAAAUAGCAAGUUAAAAU |
| | | AAGGCUAGUCCGUUAUCACGAAAGGGCACCGAGUCGGUGCU |
| G035868 | 397 | GAGACGGUUUUGUCCCUGUUGUUUUAGAGCUAGAAAUAGCAAGUUAAAAU |
| | | AAGGCUAGUCCGUUAUCACGAAAGGGCACCGAGUCGGUGCU |
| G035869 | 398 | ACUGAGAAACCCCGAUCCAGGUUUUAGAGCUAGAAAUAGCAAGUUAAAAUA |
| | | AGGCUAGUCCGUUAUCACGAAAGGGCACCGAGUCGGUGCU |
| G035870 | 399 | CAUAGCGAGGAAGCCCUGGGGUUUUAGAGCUAGAAAUAGCAAGUUAAAAU |
| | | AAGGCUAGUCCGUUAUCACGAAAGGGCACCGAGUCGGUGCU |
| G035871 | 400 | ACAUUCUAGUUCCAGGCCUGGUUUUAGAGCUAGAAAUAGCAAGUUAAAAU |
| | | AAGGCUAGUCCGUUAUCACGAAAGGGCACCGAGUCGGUGCU |
| G035872 | 401 | ACUCGGGAGGCACAGAACCAGUUUUAGAGCUAGAAAUAGCAAGUUAAAAU |
| | | AAGGCUAGUCCGUUAUCACGAAAGGGCACCGAGUCGGUGCU |
| G035873 | 402 | CAUGCCUAGAUUCACAAUCAGUUUUAGAGCUAGAAAUAGCAAGUUAAAAU |
| | | AAGGCUAGUCCGUUAUCACGAAAGGGCACCGAGUCGGUGCU |
| G035874 | 403 | GGAACUAGAAUGUCCCUCCUGUUUUAGAGCUAGAAAUAGCAAGUUAAAAU |
| | | AAGGCUAGUCCGUUAUCACGAAAGGGCACCGAGUCGGUGCU |
| G035875 | 404 | CUCAGCUAGCUGAGGCCCCUGUUUUAGAGCUAGAAAUAGCAAGUUAAAAUA |
| | | AGGCUAGUCCGUUAUCACGAAAGGGCACCGAGUCGGUGCU |
| G035876 | 405 | AAGAGACACUCCACCUGGAUGUUUUAGAGCUAGAAAUAGCAAGUUAAAAU |
| | | AAGGCUAGUCCGUUAUCACGAAAGGGCACCGAGUCGGUGCU |
| G035877 | 406 | GAAAAGUCAUUGGGAACCCCGUUUUAGAGCUAGAAAUAGCAAGUUAAAAU |
| | | AAGGCUAGUCCGUUAUCACGAAAGGGCACCGAGUCGGUGCU |
| G035878 | 407 | GCCAGUAGGUGACUUGGGACGUUUUAGAGCUAGAAAUAGCAAGUUAAAAU |
| | | AAGGCUAGUCCGUUAUCACGAAAGGGCACCGAGUCGGUGCU |
| G035879 | 408 | AUCAAGGCACCAGACUCAAUGUUUUAGAGCUAGAAAUAGCAAGUUAAAAU |
| | | AAGGCUAGUCCGUUAUCACGAAAGGGCACCGAGUCGGUGCU |
| G035880 | 409 | AUCAGGAUUGAUUAGCAGGCGUUUUAGAGCUAGAAAUAGCAAGUUAAAAU |
| | | AAGGCUAGUCCGUUAUCACGAAAGGGCACCGAGUCGGUGCU |
| G035881 | 410 | UGCCACAACUACCAGGUGGGGUUUUAGAGCUAGAAAUAGCAAGUUAAAAU |
| | | AAGGCUAGUCCGUUAUCACGAAAGGGCACCGAGUCGGUGCU |
| G035882 | 411 | GACACUCCACCUGGAUGGGAGUUUUAGAGCUAGAAAUAGCAAGUUAAAAU |
| | | AAGGCUAGUCCGUUAUCACGAAAGGGCACCGAGUCGGUGCU |
| G035883 | 412 | GCAGACCCCUGUCCCAUACAGUUUUAGAGCUAGAAAUAGCAAGUUAAAAUA |
| | | AGGCUAGUCCGUUAUCACGAAAGGGCACCGAGUCGGUGCU |
| G035884 | 413 | GGGAUCAGCUCUGACCCUGCGUUUUAGAGCUAGAAAUAGCAAGUUAAAAUA |
| | | AGGCUAGUCCGUUAUCACGAAAGGGCACCGAGUCGGUGCU |
| G035885 | 414 | UCAGCUAGCUGAGGCCCCUGGUUUUAGAGCUAGAAAUAGCAAGUUAAAAUA |
| | | AGGCUAGUCCGUUAUCACGAAAGGGCACCGAGUCGGUGCU |
| G035886 | 415 | CUGGGGCUCUGUAUGCCAGAGUUUUAGAGCUAGAAAUAGCAAGUUAAAAU |
| | | AAGGCUAGUCCGUUAUCACGAAAGGGCACCGAGUCGGUGCU |
| G035887 | 416 | GAGUAACAGUGCAGUGUGCUGUUUUAGAGCUAGAAAUAGCAAGUUAAAAU |
| | | AAGGCUAGUCCGUUAUCACGAAAGGGCACCGAGUCGGUGCU |
| G035888 | 417 | GUGCUUCUGCAGGGUUGCCAGUUUUAGAGCUAGAAAUAGCAAGUUAAAAU |
| | | AAGGCUAGUCCGUUAUCACGAAAGGGCACCGAGUCGGUGCU |
| G035889 | 418 | UCAGAGCUGAUCCCCUCUGUGUUUUAGAGCUAGAAAUAGCAAGUUAAAAUA |
| | | AGGCUAGUCCGUUAUCACGAAAGGGCACCGAGUCGGUGCU |
| G035890 | 419 | CGGGAGGCACAGAACCAAGGGUUUUAGAGCUAGAAAUAGCAAGUUAAAAU |
| | | AAGGCUAGUCCGUUAUCACGAAAGGGCACCGAGUCGGUGCU |
| G035891 | 420 | UGUCUCCCUGAAAAGUCAUUGUUUUAGAGCUAGAAAUAGCAAGUUAAAAU |
| | | AAGGCUAGUCCGUUAUCACGAAAGGGCACCGAGUCGGUGCU |
| G035892 | 421 | AAAGUCAUUGGGAACCCCUGGUUUUAGAGCUAGAAAUAGCAAGUUAAAAU |
| | | AAGGCUAGUCCGUUAUCACGAAAGGGCACCGAGUCGGUGCU |
| G035893 | 422 | GUGGUGGAUGCUCCUGCCCCGUUUUAGAGCUAGAAAUAGCAAGUUAAAAUA |
| | | AGGCUAGUCCGUUAUCACGAAAGGGCACCGAGUCGGUGCU |
| G035894 | 423 | GUGGAAUUCUGGCAUCUAGUGUUUUAGAGCUAGAAAUAGCAAGUUAAAAU |
| | | AAGGCUAGUCCGUUAUCACGAAAGGGCACCGAGUCGGUGCU |
| G035895 | 424 | CCUGUGGCCAGUAGGUGACUGUUUUAGAGCUAGAAAUAGCAAGUUAAAAU |
| | | AAGGCUAGUCCGUUAUCACGAAAGGGCACCGAGUCGGUGCU |
| G035896 | 425 | AUGGAUAGAAAAACGGUGAAGUUUUAGAGCUAGAAAUAGCAAGUUAAAAU |
| | | AAGGCUAGUCCGUUAUCACGAAAGGGCACCGAGUCGGUGCU |
| G035897 | 426 | CUCCACCUGGAUGGGAAGGUGUUUUAGAGCUAGAAAUAGCAAGUUAAAAU |
| | | AAGGCUAGUCCGUUAUCACGAAAGGGCACCGAGUCGGUGCU |
| G035898 | 427 | ACUCCACCUGGAUGGGAAGGGUUUUAGAGCUAGAAAUAGCAAGUUAAAAU |
| | | AAGGCUAGUCCGUUAUCACGAAAGGGCACCGAGUCGGUGCU |

TABLE 5
Human ASS1 Intron 1 Guide Sequences.

| Guide RNA ID# | Guide Sequence | SEQ ID NO: |
|---|---|---|
| G033085 | CAUAGGGACUAAUGCGUGGU | 430 |
| G033086 | CACCGUCACUCAUUCAAGCC | 431 |
| G033087 | GUGGCCAGGUUCAGUCGAAG | 432 |
| G033088 | AAUGCGUGGUGGGUCCCCAU | 433 |
| G033089 | AGCUCCUCUUCGACUGAACC | 434 |
| G033090 | CCAUAGGGACUAAUGCGUGG | 435 |
| G033091 | CAGCCAUAGGGACUAAUGCG | 436 |
| G033092 | UCAUUCGGCUCACCAGACCC | 437 |
| G033093 | GGUCUGGUGAGCCGAAUGAA | 438 |
| G033094 | UGGCUUGUUUGCCGGCAUAU | 439 |
| G033095 | AGGAGGGCUUACCCCCAGUA | 440 |
| G033096 | GCCGGCAAACAAGCCACUUU | 441 |
| G033097 | GAGCUGGGCGUAUAGACCCC | 442 |
| G033098 | GCAUCAUGGGGUGCGAGGCU | 443 |
| G033099 | AGCUGGGCGUAUAGACCCCU | 444 |
| G033100 | UAAUGCGUGGUGGGUCCCCA | 445 |
| G033101 | AAGGAGGGCUUACCCCCAGU | 446 |
| G033102 | CAUCAUGGGGUGCGAGGCUU | 447 |
| G033103 | UCUCGAAGCCUGUCUUGAAC | 448 |
| G033104 | UUGGGAGCUGGAUUGUAGCG | 449 |
| G033105 | GGGUCUGGUGAGCCGAAUGA | 450 |
| G033106 | AGUCCCUAUGGCUGCAUCAU | 451 |
| G033107 | UGGGGGUAAGCCCUCCUUUU | 452 |
| G033108 | UUACCCCCAGUAGGGCCCAU | 453 |
| G033109 | UUGAGAGGGAUGACUCCUAU | 454 |
| G033110 | GGGGGUAAGCCCUCCUUUUU | 455 |
| G033111 | UGACAAGCUAAUCUUUGCAC | 456 |
| G033112 | GCCCAAUGCCCUUGUCAUUC | 457 |
| G033113 | UACCCCCAGUAGGGCCCAUG | 458 |
| G033114 | CCUAGUCAAAGGCCAUGCCU | 459 |
| G033115 | CACAGCGGGGCAAUCAGAGC | 460 |
| G033116 | UAGACCCCUGGGCUACCACC | 461 |
| G033117 | AUCCCGCCCACUGAAGGAGU | 462 |
| G033118 | UGCAGGCUGACAGCAUACUC | 463 |
| G033119 | CAGAACCAGGUGAUCCCCCA | 464 |
| G033120 | GGGUGUAUCCAUCUCACUGU | 465 |
| G033121 | GGGUCCCCAUGGGCCCUACU | 466 |
| G033122 | CAAGGCAUGGCCUUUGACUA | 467 |
| G033123 | GAUCUAUCUCCAACUCUACU | 468 |
| G033124 | CAGUGAGAUGGAUACACCCU | 469 |
| G033125 | GAAGAAAUCCCGCCCACUGA | 470 |
| G033126 | AUCCCCCAGGUGGUAGCCCA | 471 |
| G033127 | GUCCCCAUGGGCCCUACUGG | 472 |
| G033128 | AAUCCCCCAGGUGGUAGCCC | 473 |
| G033129 | CAUGAGCCUACUCCUUCAGU | 474 |
| G033130 | UCCUAGUUCCAGUUCAAGAC | 475 |
| G033131 | GGUCCCCAUGGGCCCUACUG | 476 |
| G033132 | CCACCACGCAUUAGUCCCUA | 477 |
| G033133 | UGGGUGGACCCCAUUCCACA | 478 |
| G033134 | AGUUAGACAUGCCAAUAUGC | 479 |
| G033135 | AGUGCAAAGAUUAGCUUGUC | 480 |
| G033136 | UGGGUCCCCAUGGGCCCUAC | 481 |
| G033137 | AUGCCCUUGUCAUUCAGGUU | 482 |
| G033138 | UAGUCCCUAUGGCUGCAUCA | 483 |
| G033139 | GCCUGUCUUGAACUGGAACU | 484 |
| G033140 | CAACCAUCUGCUGUCAUUGC | 485 |
| G033141 | ACAGUGAGAUGGAUACACCC | 486 |
| G033142 | UUUGAGAGGGAUGACUCCUA | 487 |
| G033143 | GCCAAAAGUGGCUUGUUUGC | 488 |
| G033144 | GAUUGUAGCGUGGUGAUGGC | 489 |
| G033145 | CCACUGAAGGAGUAGGCUCA | 490 |
| G033146 | GGACAGGUUGCAGGACACGC | 491 |
| G033147 | GCCUGAAGCCAUCCUACCCC | 492 |
| G033148 | UCACUGUAGGUAGCACAGCG | 493 |
| G033149 | CCAAGGCAUGGCCUUUGACU | 494 |
| G033150 | ACCUGAAUGACAAGGGCAUU | 495 |
| G033151 | CCAUGAGCCUACUCCUUCAG | 496 |
| G033152 | AAACAUGGCAGUGUCUAGAC | 497 |
| G033153 | GGGAUGACUCCUAUGGGCCC | 498 |
| G033154 | GCUUCCAUGGGUUGGAGCAU | 499 |
| G033155 | CUUACCCCCAGUAGGGCCCA | 500 |
| G033156 | GUGAACUCAGGGCUCCCCCC | 501 |
| G033157 | GUCCCUAUGGCUGCAUCAUG | 502 |
| G033158 | GGCAUGGCCUUUGACUAGGG | 503 |
| G033159 | GCACCCCAUGAUGCAGCCAU | 504 |
| G033160 | AUGAGUGACGGUGAGCAGAC | 505 |
| G033161 | UGGAAGAAUCUCACCUCUCC | 506 |
| G033162 | CUGGGUGGACCCCAUUCCAC | 507 |
| G033163 | UGACAGCAUACUCUGGAGAU | 508 |
| G033164 | CAGCUCUCCUCCCUAGUCAA | 509 |
| G033165 | CCAGAACCAGGUGAUCCCCC | 510 |
| G033166 | AGCCAGGUGAUGGAGGCGCG | 511 |
| G033167 | CCUUUCCUUUUUGCGUAAGU | 512 |
| G033168 | UCUGGAAGGCAUCUCAAGGA | 513 |
| G033169 | AACAUGGCAGUGUCUAGACA | 514 |
| G033170 | UGAACUCAGGGCUCCCCCCA | 515 |
| G033171 | AGACCCCUGGGCUACCACCU | 516 |
| G033172 | UCAGCCAAUGCUCCAACCCA | 517 |

TABLE 6
Human ASS1 Intron 1 sgRNA Sequences with scaffold B.

| Guide | SEQ | |
|---|---|---|
| RNA ID# | ID NO: | Full Sequence |
| G033085 | 606 | CAUAGGGACUAAUGCGUGGUGUUUUAGAGCUAGAAAUAGCAAGUUAAAAU |
| | | AAGGCUAGUCCGUUAUCACGAAAGGGCACCGAGUCGGUGCU |
| G033086 | 607 | CACCGUCACUCAUUCAAGCCGUUUUAGAGCUAGAAAUAGCAAGUUAAAAUA |
| | | AGGCUAGUCCGUUAUCACGAAAGGGCACCGAGUCGGUGCU |
| G033087 | 608 | GUGGCCAGGUUCAGUCGAAGGUUUUAGAGCUAGAAAUAGCAAGUUAAAAU |
| | | AAGGCUAGUCCGUUAUCACGAAAGGGCACCGAGUCGGUGCU |
| G033088 | 609 | AAUGCGUGGUGGGUCCCCAUGUUUUAGAGCUAGAAAUAGCAAGUUAAAAU |
| | | AAGGCUAGUCCGUUAUCACGAAAGGGCACCGAGUCGGUGCU |
| G033089 | 610 | AGCUCCUCUUCGACUGAACCGUUUUAGAGCUAGAAAUAGCAAGUUAAAAUA |
| | | AGGCUAGUCCGUUAUCACGAAAGGGCACCGAGUCGGUGCU |
| G033090 | 611 | CCAUAGGGACUAAUGCGUGGGUUUUAGAGCUAGAAAUAGCAAGUUAAAAU |
| | | AAGGCUAGUCCGUUAUCACGAAAGGGCACCGAGUCGGUGCU |
| G033091 | 612 | CAGCCAUAGGGACUAAUGCGGUUUUAGAGCUAGAAAUAGCAAGUUAAAAU |
| | | AAGGCUAGUCCGUUAUCACGAAAGGGCACCGAGUCGGUGCU |
| G033092 | 613 | UCAUUCGGCUCACCAGACCCGUUUUAGAGCUAGAAAUAGCAAGUUAAAAUA |
| | | AGGCUAGUCCGUUAUCACGAAAGGGCACCGAGUCGGUGCU |
| G033093 | 614 | GGUCUGGUGAGCCGAAUGAAGUUUUAGAGCUAGAAAUAGCAAGUUAAAAU |
| | | AAGGCUAGUCCGUUAUCACGAAAGGGCACCGAGUCGGUGCU |
| G033094 | 615 | UGGCUUGUUUGCCGGCAUAUGUUUUAGAGCUAGAAAUAGCAAGUUAAAAU |
| | | AAGGCUAGUCCGUUAUCACGAAAGGGCACCGAGUCGGUGCU |
| G033095 | 616 | AGGAGGGCUUACCCCCAGUAGUUUUAGAGCUAGAAAUAGCAAGUUAAAAU |
| | | AAGGCUAGUCCGUUAUCACGAAAGGGCACCGAGUCGGUGCU |
| G033096 | 617 | GCCGGCAAACAAGCCACUUUGUUUUAGAGCUAGAAAUAGCAAGUUAAAAUA |
| | | AGGCUAGUCCGUUAUCACGAAAGGGCACCGAGUCGGUGCU |
| G033097 | 618 | GAGCUGGGCGUAUAGACCCCGUUUUAGAGCUAGAAAUAGCAAGUUAAAAU |
| | | AAGGCUAGUCCGUUAUCACGAAAGGGCACCGAGUCGGUGCU |
| G033098 | 619 | GCAUCAUGGGGUGCGAGGCUGUUUUAGAGCUAGAAAUAGCAAGUUAAAAU |
| | | AAGGCUAGUCCGUUAUCACGAAAGGGCACCGAGUCGGUGCU |
| G033099 | 620 | AGCUGGGCGUAUAGACCCCUGUUUUAGAGCUAGAAAUAGCAAGUUAAAAU |
| | | AAGGCUAGUCCGUUAUCACGAAAGGGCACCGAGUCGGUGCU |
| G033100 | 621 | UAAUGCGUGGUGGGUCCCCAGUUUUAGAGCUAGAAAUAGCAAGUUAAAAU |
| | | AAGGCUAGUCCGUUAUCACGAAAGGGCACCGAGUCGGUGCU |
| G033101 | 622 | AAGGAGGGCUUACCCCCAGUGUUUUAGAGCUAGAAAUAGCAAGUUAAAAU |
| | | AAGGCUAGUCCGUUAUCACGAAAGGGCACCGAGUCGGUGCU |
| G033102 | 623 | CAUCAUGGGGUGCGAGGCUUGUUUUAGAGCUAGAAAUAGCAAGUUAAAAU |
| | | AAGGCUAGUCCGUUAUCACGAAAGGGCACCGAGUCGGUGCU |
| G033103 | 624 | UCUCGAAGCCUGUCUUGAACGUUUUAGAGCUAGAAAUAGCAAGUUAAAAU |
| | | AAGGCUAGUCCGUUAUCACGAAAGGGCACCGAGUCGGUGCU |
| G033104 | 625 | UUGGGAGCUGGAUUGUAGCGGUUUUAGAGCUAGAAAUAGCAAGUUAAAAU |
| | | AAGGCUAGUCCGUUAUCACGAAAGGGCACCGAGUCGGUGCU |
| G033105 | 626 | GGGUCUGGUGAGCCGAAUGAGUUUUAGAGCUAGAAAUAGCAAGUUAAAAU |
| | | AAGGCUAGUCCGUUAUCACGAAAGGGCACCGAGUCGGUGCU |
| G033106 | 627 | AGUCCCUAUGGCUGCAUCAUGUUUUAGAGCUAGAAAUAGCAAGUUAAAAU |
| | | AAGGCUAGUCCGUUAUCACGAAAGGGCACCGAGUCGGUGCU |
| G033107 | 628 | UGGGGGUAAGCCCUCCUUUUGUUUUAGAGCUAGAAAUAGCAAGUUAAAAU |
| | | AAGGCUAGUCCGUUAUCACGAAAGGGCACCGAGUCGGUGCU |
| G033108 | 629 | UUACCCCCAGUAGGGCCCAUGUUUUAGAGCUAGAAAUAGCAAGUUAAAAUA |
| | | AGGCUAGUCCGUUAUCACGAAAGGGCACCGAGUCGGUGCU |
| G033109 | 630 | UUGAGAGGGAUGACUCCUAUGUUUUAGAGCUAGAAAUAGCAAGUUAAAAU |
| | | AAGGCUAGUCCGUUAUCACGAAAGGGCACCGAGUCGGUGCU |
| G033110 | 631 | GGGGGUAAGCCCUCCUUUUUGUUUUAGAGCUAGAAAUAGCAAGUUAAAAU |
| | | AAGGCUAGUCCGUUAUCACGAAAGGGCACCGAGUCGGUGCU |
| G033111 | 632 | UGACAAGCUAAUCUUUGCACGUUUUAGAGCUAGAAAUAGCAAGUUAAAAU |
| | | AAGGCUAGUCCGUUAUCACGAAAGGGCACCGAGUCGGUGCU |
| G033112 | 633 | GCCCAAUGCCCUUGUCAUUCGUUUUAGAGCUAGAAAUAGCAAGUUAAAAUA |
| | | AGGCUAGUCCGUUAUCACGAAAGGGCACCGAGUCGGUGCU |
| G033113 | 634 | UACCCCCAGUAGGGCCCAUGGUUUUAGAGCUAGAAAUAGCAAGUUAAAAUA |
| | | AGGCUAGUCCGUUAUCACGAAAGGGCACCGAGUCGGUGCU |
| G033114 | 635 | CCUAGUCAAAGGCCAUGCCUGUUUUAGAGCUAGAAAUAGCAAGUUAAAAUA |
| | | AGGCUAGUCCGUUAUCACGAAAGGGCACCGAGUCGGUGCU |
| G033115 | 636 | CACAGCGGGGCAAUCAGAGCGUUUUAGAGCUAGAAAUAGCAAGUUAAAAU |
| | | AAGGCUAGUCCGUUAUCACGAAAGGGCACCGAGUCGGUGCU |
| G033116 | 637 | UAGACCCCUGGGCUACCACCGUUUUAGAGCUAGAAAUAGCAAGUUAAAAUA |
| | | AGGCUAGUCCGUUAUCACGAAAGGGCACCGAGUCGGUGCU |
| G033117 | 638 | AUCCCGCCCACUGAAGGAGUGUUUUAGAGCUAGAAAUAGCAAGUUAAAAU |
| | | AAGGCUAGUCCGUUAUCACGAAAGGGCACCGAGUCGGUGCU |
| G033118 | 639 | UGCAGGCUGACAGCAUACUCGUUUUAGAGCUAGAAAUAGCAAGUUAAAAU |
| | | AAGGCUAGUCCGUUAUCACGAAAGGGCACCGAGUCGGUGCU |
| G033119 | 640 | CAGAACCAGGUGAUCCCCCAGUUUUAGAGCUAGAAAUAGCAAGUUAAAAUA |
| | | AGGCUAGUCCGUUAUCACGAAAGGGCACCGAGUCGGUGCU |
| G033120 | 641 | GGGUGUAUCCAUCUCACUGUGUUUUAGAGCUAGAAAUAGCAAGUUAAAAU |
| | | AAGGCUAGUCCGUUAUCACGAAAGGGCACCGAGUCGGUGCU |
| G033121 | 642 | GGGUCCCCAUGGGCCCUACUGUUUUAGAGCUAGAAAUAGCAAGUUAAAAUA |
| | | AGGCUAGUCCGUUAUCACGAAAGGGCACCGAGUCGGUGCU |
| G033122 | 643 | CAAGGCAUGGCCUUUGACUAGUUUUAGAGCUAGAAAUAGCAAGUUAAAAU |
| | | AAGGCUAGUCCGUUAUCACGAAAGGGCACCGAGUCGGUGCU |
| G033123 | 644 | GAUCUAUCUCCAACUCUACUGUUUUAGAGCUAGAAAUAGCAAGUUAAAAUA |
| | | AGGCUAGUCCGUUAUCACGAAAGGGCACCGAGUCGGUGCU |
| G033124 | 645 | CAGUGAGAUGGAUACACCCUGUUUUAGAGCUAGAAAUAGCAAGUUAAAAU |
| | | AAGGCUAGUCCGUUAUCACGAAAGGGCACCGAGUCGGUGCU |
| G033125 | 646 | GAAGAAAUCCCGCCCACUGAGUUUUAGAGCUAGAAAUAGCAAGUUAAAAUA |
| | | AGGCUAGUCCGUUAUCACGAAAGGGCACCGAGUCGGUGCU |
| G033126 | 647 | AUCCCCCAGGUGGUAGCCCAGUUUUAGAGCUAGAAAUAGCAAGUUAAAAUA |
| | | AGGCUAGUCCGUUAUCACGAAAGGGCACCGAGUCGGUGCU |
| G033127 | 648 | GUCCCCAUGGGCCCUACUGGGUUUUAGAGCUAGAAAUAGCAAGUUAAAAUA |
| | | AGGCUAGUCCGUUAUCACGAAAGGGCACCGAGUCGGUGCU |
| G033128 | 649 | AAUCCCCCAGGUGGUAGCCCGUUUUAGAGCUAGAAAUAGCAAGUUAAAAUA |
| | | AGGCUAGUCCGUUAUCACGAAAGGGCACCGAGUCGGUGCU |
| G033129 | 650 | CAUGAGCCUACUCCUUCAGUGUUUUAGAGCUAGAAAUAGCAAGUUAAAAUA |
| | | AGGCUAGUCCGUUAUCACGAAAGGGCACCGAGUCGGUGCU |
| G033130 | 651 | UCCUAGUUCCAGUUCAAGACGUUUUAGAGCUAGAAAUAGCAAGUUAAAAU |
| | | AAGGCUAGUCCGUUAUCACGAAAGGGCACCGAGUCGGUGCU |
| G033131 | 652 | GGUCCCCAUGGGCCCUACUGGUUUUAGAGCUAGAAAUAGCAAGUUAAAAUA |
| | | AGGCUAGUCCGUUAUCACGAAAGGGCACCGAGUCGGUGCU |
| G033132 | 653 | CCACCACGCAUUAGUCCCUAGUUUUAGAGCUAGAAAUAGCAAGUUAAAAUA |
| | | AGGCUAGUCCGUUAUCACGAAAGGGCACCGAGUCGGUGCU |
| G033133 | 654 | UGGGUGGACCCCAUUCCACAGUUUUAGAGCUAGAAAUAGCAAGUUAAAAUA |
| | | AGGCUAGUCCGUUAUCACGAAAGGGCACCGAGUCGGUGCU |
| G033134 | 655 | AGUUAGACAUGCCAAUAUGCGUUUUAGAGCUAGAAAUAGCAAGUUAAAAU |
| | | AAGGCUAGUCCGUUAUCACGAAAGGGCACCGAGUCGGUGCU |
| G033135 | 656 | AGUGCAAAGAUUAGCUUGUCGUUUUAGAGCUAGAAAUAGCAAGUUAAAAU |
| | | AAGGCUAGUCCGUUAUCACGAAAGGGCACCGAGUCGGUGCU |
| G033136 | 657 | UGGGUCCCCAUGGGCCCUACGUUUUAGAGCUAGAAAUAGCAAGUUAAAAUA |
| | | AGGCUAGUCCGUUAUCACGAAAGGGCACCGAGUCGGUGCU |
| G033137 | 658 | AUGCCCUUGUCAUUCAGGUUGUUUUAGAGCUAGAAAUAGCAAGUUAAAAU |
| | | AAGGCUAGUCCGUUAUCACGAAAGGGCACCGAGUCGGUGCU |
| G033138 | 659 | UAGUCCCUAUGGCUGCAUCAGUUUUAGAGCUAGAAAUAGCAAGUUAAAAU |
| | | AAGGCUAGUCCGUUAUCACGAAAGGGCACCGAGUCGGUGCU |
| G033139 | 660 | GCCUGUCUUGAACUGGAACUGUUUUAGAGCUAGAAAUAGCAAGUUAAAAU |
| | | AAGGCUAGUCCGUUAUCACGAAAGGGCACCGAGUCGGUGCU |
| G033140 | 661 | CAACCAUCUGCUGUCAUUGCGUUUUAGAGCUAGAAAUAGCAAGUUAAAAUA |
| | | AGGCUAGUCCGUUAUCACGAAAGGGCACCGAGUCGGUGCU |
| G033141 | 662 | ACAGUGAGAUGGAUACACCCGUUUUAGAGCUAGAAAUAGCAAGUUAAAAU |
| | | AAGGCUAGUCCGUUAUCACGAAAGGGCACCGAGUCGGUGCU |
| G033142 | 663 | UUUGAGAGGGAUGACUCCUAGUUUUAGAGCUAGAAAUAGCAAGUUAAAAU |
| | | AAGGCUAGUCCGUUAUCACGAAAGGGCACCGAGUCGGUGCU |
| G033143 | 664 | GCCAAAAGUGGCUUGUUUGCGUUUUAGAGCUAGAAAUAGCAAGUUAAAAU |
| | | AAGGCUAGUCCGUUAUCACGAAAGGGCACCGAGUCGGUGCU |
| G033144 | 665 | GAUUGUAGCGUGGUGAUGGCGUUUUAGAGCUAGAAAUAGCAAGUUAAAAU |
| | | AAGGCUAGUCCGUUAUCACGAAAGGGCACCGAGUCGGUGCU |
| G033145 | 666 | CCACUGAAGGAGUAGGCUCAGUUUUAGAGCUAGAAAUAGCAAGUUAAAAU |
| | | AAGGCUAGUCCGUUAUCACGAAAGGGCACCGAGUCGGUGCU |
| G033146 | 667 | GGACAGGUUGCAGGACACGCGUUUUAGAGCUAGAAAUAGCAAGUUAAAAU |
| | | AAGGCUAGUCCGUUAUCACGAAAGGGCACCGAGUCGGUGCU |
| G033147 | 668 | GCCUGAAGCCAUCCUACCCCGUUUUAGAGCUAGAAAUAGCAAGUUAAAAUA |
| | | AGGCUAGUCCGUUAUCACGAAAGGGCACCGAGUCGGUGCU |
| G033148 | 669 | UCACUGUAGGUAGCACAGCGGUUUUAGAGCUAGAAAUAGCAAGUUAAAAU |
| | | AAGGCUAGUCCGUUAUCACGAAAGGGCACCGAGUCGGUGCU |
| G033149 | 670 | CCAAGGCAUGGCCUUUGACUGUUUUAGAGCUAGAAAUAGCAAGUUAAAAU |
| | | AAGGCUAGUCCGUUAUCACGAAAGGGCACCGAGUCGGUGCU |
| G033150 | 671 | ACCUGAAUGACAAGGGCAUUGUUUUAGAGCUAGAAAUAGCAAGUUAAAAU |
| | | AAGGCUAGUCCGUUAUCACGAAAGGGCACCGAGUCGGUGCU |
| G033151 | 672 | CCAUGAGCCUACUCCUUCAGGUUUUAGAGCUAGAAAUAGCAAGUUAAAAUA |
| | | AGGCUAGUCCGUUAUCACGAAAGGGCACCGAGUCGGUGCU |
| G033152 | 673 | AAACAUGGCAGUGUCUAGACGUUUUAGAGCUAGAAAUAGCAAGUUAAAAU |
| | | AAGGCUAGUCCGUUAUCACGAAAGGGCACCGAGUCGGUGCU |
| G033153 | 674 | GGGAUGACUCCUAUGGGCCCGUUUUAGAGCUAGAAAUAGCAAGUUAAAAU |
| | | AAGGCUAGUCCGUUAUCACGAAAGGGCACCGAGUCGGUGCU |
| G033154 | 675 | GCUUCCAUGGGUUGGAGCAUGUUUUAGAGCUAGAAAUAGCAAGUUAAAAU |
| | | AAGGCUAGUCCGUUAUCACGAAAGGGCACCGAGUCGGUGCU |
| G033155 | 676 | CUUACCCCCAGUAGGGCCCAGUUUUAGAGCUAGAAAUAGCAAGUUAAAAUA |
| | | AGGCUAGUCCGUUAUCACGAAAGGGCACCGAGUCGGUGCU |
| G033156 | 677 | GUGAACUCAGGGCUCCCCCCGUUUUAGAGCUAGAAAUAGCAAGUUAAAAUA |
| | | AGGCUAGUCCGUUAUCACGAAAGGGCACCGAGUCGGUGCU |
| G033157 | 678 | GUCCCUAUGGCUGCAUCAUGGUUUUAGAGCUAGAAAUAGCAAGUUAAAAU |
| | | AAGGCUAGUCCGUUAUCACGAAAGGGCACCGAGUCGGUGCU |
| G033158 | 679 | GGCAUGGCCUUUGACUAGGGGUUUUAGAGCUAGAAAUAGCAAGUUAAAAU |
| | | AAGGCUAGUCCGUUAUCACGAAAGGGCACCGAGUCGGUGCU |
| G033159 | 680 | GCACCCCAUGAUGCAGCCAUGUUUUAGAGCUAGAAAUAGCAAGUUAAAAUA |
| | | AGGCUAGUCCGUUAUCACGAAAGGGCACCGAGUCGGUGCU |
| G033160 | 681 | AUGAGUGACGGUGAGCAGACGUUUUAGAGCUAGAAAUAGCAAGUUAAAAU |
| | | AAGGCUAGUCCGUUAUCACGAAAGGGCACCGAGUCGGUGCU |
| G033161 | 682 | UGGAAGAAUCUCACCUCUCCGUUUUAGAGCUAGAAAUAGCAAGUUAAAAUA |
| | | AGGCUAGUCCGUUAUCACGAAAGGGCACCGAGUCGGUGCU |
| G033162 | 683 | CUGGGUGGACCCCAUUCCACGUUUUAGAGCUAGAAAUAGCAAGUUAAAAUA |
| | | AGGCUAGUCCGUUAUCACGAAAGGGCACCGAGUCGGUGCU |
| G033163 | 684 | UGACAGCAUACUCUGGAGAUGUUUUAGAGCUAGAAAUAGCAAGUUAAAAU |
| | | AAGGCUAGUCCGUUAUCACGAAAGGGCACCGAGUCGGUGCU |
| G033164 | 685 | CAGCUCUCCUCCCUAGUCAAGUUUUAGAGCUAGAAAUAGCAAGUUAAAAUA |
| | | AGGCUAGUCCGUUAUCACGAAAGGGCACCGAGUCGGUGCU |
| G033165 | 686 | CCAGAACCAGGUGAUCCCCCGUUUUAGAGCUAGAAAUAGCAAGUUAAAAUA |
| | | AGGCUAGUCCGUUAUCACGAAAGGGCACCGAGUCGGUGCU |
| G033166 | 687 | AGCCAGGUGAUGGAGGCGCGGUUUUAGAGCUAGAAAUAGCAAGUUAAAAU |
| | | AAGGCUAGUCCGUUAUCACGAAAGGGCACCGAGUCGGUGCU |
| G033167 | 688 | CCUUUCCUUUUUGCGUAAGUGUUUUAGAGCUAGAAAUAGCAAGUUAAAAU |
| | | AAGGCUAGUCCGUUAUCACGAAAGGGCACCGAGUCGGUGCU |
| G033168 | 689 | UCUGGAAGGCAUCUCAAGGAGUUUUAGAGCUAGAAAUAGCAAGUUAAAAU |
| | | AAGGCUAGUCCGUUAUCACGAAAGGGCACCGAGUCGGUGCU |
| G033169 | 690 | AACAUGGCAGUGUCUAGACAGUUUUAGAGCUAGAAAUAGCAAGUUAAAAU |
| | | AAGGCUAGUCCGUUAUCACGAAAGGGCACCGAGUCGGUGCU |
| G033170 | 691 | UGAACUCAGGGCUCCCCCCAGUUUUAGAGCUAGAAAUAGCAAGUUAAAAUA |
| | | AGGCUAGUCCGUUAUCACGAAAGGGCACCGAGUCGGUGCU |
| G033171 | 692 | AGACCCCUGGGCUACCACCUGUUUUAGAGCUAGAAAUAGCAAGUUAAAAUA |
| | | AGGCUAGUCCGUUAUCACGAAAGGGCACCGAGUCGGUGCU |
| G033172 | 693 | UCAGCCAAUGCUCCAACCCAGUUUUAGAGCUAGAAAUAGCAAGUUAAAAUA |
| | | AGGCUAGUCCGUUAUCACGAAAGGGCACCGAGUCGGUGCU |

TABLE 7
Mouse Ass1 Intron 1 Guide Sequences.

| | | SEQ |
|---|---|---|
| Guide RNA ID | Guide Sequence | ID NO: |
| mAss1_in1_g1/G033631 | AGACAUCACUAAUCGAGCCG | 207 |
| mAss1_in1_g2/G033632 | ACCGGUAGGGACCCGGCAAC | 208 |
| mAss1_in1_g3/G033633 | UGUGGACUUGGGCCCGUUGC | 209 |
| mAss1_in1_g4/G033634 | CCUAUGCAGCCGCUCGUACU | 210 |
| mAss1_in1_g5/G033635 | GAAUCUGUAGUCGUGAUGUC | 211 |
| mAss1_in1_g6/G033636 | CAAAACUGCAUCUGGCGUCC | 212 |

TABLE 8
Mouse Ass1 Intron 1 sgRNA Sequences with scaffold A.

| Corr. to | SEQ | |
|---|---|---|
| Guide RNA ID | ID NO: | Full Sequence |
| mAss1_in1_g1/ | 219 | AGACAUCACUAAUCGAGCCGGUUUUAGAGCUAGAAAUAGCAAGUUAAA |
| G033631 | | AUAAGGCUAGUCCGUUAUCAACUUGAAAAAGUGGCACCGAGUCGGUGCU |
| | | UUU |
| mAss1_in1_g2/ | 220 | ACCGGUAGGGACCCGGCAACGUUUUAGAGCUAGAAAUAGCAAGUUAAA |
| G033632 | | AUAAGGCUAGUCCGUUAUCAACUUGAAAAAGUGGCACCGAGUCGGUGCU |
| | | UUU |
| mAss1_in1_g3/ | 221 | UGUGGACUUGGGCCCGUUGCGUUUUAGAGCUAGAAAUAGCAAGUUAAA |
| G033633 | | AUAAGGCUAGUCCGUUAUCAACUUGAAAAAGUGGCACCGAGUCGGUGCU |
| | | UUU |
| mAss1_in1_g4/ | 222 | CCUAUGCAGCCGCUCGUACUGUUUUAGAGCUAGAAAUAGCAAGUUAAAA |
| G033634 | | UAAGGCUAGUCCGUUAUCAACUUGAAAAAGUGGCACCGAGUCGGUGCUU |
| | | UU |
| mAss1_in1_g5/ | 223 | GAAUCUGUAGUCGUGAUGUCGUUUUAGAGCUAGAAAUAGCAAGUUAAA |
| G033635 | | AUAAGGCUAGUCCGUUAUCAACUUGAAAAAGUGGCACCGAGUCGGUGCU |
| | | UUU |
| mAss1_in1_g6/ | 224 | CAAAACUGCAUCUGGCGUCCGUUUUAGAGCUAGAAAUAGCAAGUUAAA |
| G033636 | | AUAAGGCUAGUCCGUUAUCAACUUGAAAAAGUGGCACCGAGUCGGUGCU |
| | | UUU |

TABLE 9
Mouse Ass1 Intron 1 sgRNA Sequences with scaffold B.

| Guide RNA ID | SEQ ID NO: | Full Sequence |
|---|---|---|
| mAss1_in1_g1/ | 697 | AGACAUCACUAAUCGAGCCGGUUUUAGAGCUAGAAAUAGCAAGUUAAA |
| G033631 | | AUAAGGCUAGUCCGUUAUCACGAAAGGGCACCGAGUCGGUGCU |
| mAss1_in1_g2/ | 698 | ACCGGUAGGGACCCGGCAACGUUUUAGAGCUAGAAAUAGCAAGUUAAA |
| G033632 | | AUAAGGCUAGUCCGUUAUCACGAAAGGGCACCGAGUCGGUGCU |
| mAss1_in1_g3/ | 699 | UGUGGACUUGGGCCCGUUGCGUUUUAGAGCUAGAAAUAGCAAGUUAAA |
| G033633 | | AUAAGGCUAGUCCGUUAUCACGAAAGGGCACCGAGUCGGUGCU |
| mAss1_in1_g4/ | 700 | CCUAUGCAGCCGCUCGUACUGUUUUAGAGCUAGAAAUAGCAAGUUAAAA |
| G033634 | | UAAGGCUAGUCCGUUAUCACGAAAGGGCACCGAGUCGGUGCU |
| mAss1_in1_g5/ | 701 | GAAUCUGUAGUCGUGAUGUCGUUUUAGAGCUAGAAAUAGCAAGUUAAA |
| G033635 | | AUAAGGCUAGUCCGUUAUCACGAAAGGGCACCGAGUCGGUGCU |
| mAss1_in1_g6/ | 702 | CAAAACUGCAUCUGGCGUCCGUUUUAGAGCUAGAAAUAGCAAGUUAAA |
| G033636 | | AUAAGGCUAGUCCGUUAUCACGAAAGGGCACCGAGUCGGUGCU |

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471(7340):602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, at least 97%, at least 98%, at least 99%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences (e.g., for use with S. pyogenes Cas9) comprise, consist essentially of, or consist of:

GUUUUAGAGCUAGAAAUAGCAAGUUAAAAUAAGGCUAGUCCGUUAUCAA
CUUGAAAAAGUGGCACCGAGUCGGUGCU
(version 1; SEQ ID NO: 22);
GUUGGAACCAUUCAAAACAGCAUAGCAAGUUAAAAUAAGGCUAGUCCGU
UAUCAACUUGAAAAAGUGGCACCGAGUCGGUGC
(version 2; SEQ ID NO: 23);
GUUUUAGAGCUAGAAAUAGCAAGUUAAAAUAAGGCUAGUCCGUUAUCAA
CUUGAAAAAGUGGCACCGAGUCGGUGC
(version 3; SEQ ID NO: 24);
and
GUUUAAGAGCUAUGCUGGAAACAGCAUAGCAAGUUUAAAUAAGGCUAGU
CCGUUAUCAACUUGAAAAAGUGGCACCGAGUCGGUGC
(version 4; SEQ ID NO: 25);
GUUUUAGAGCUAGAAAUAGCAAGUUAAAAUAAGGCUAGUCCGUUAUCAA
CUUGAAAAAGUGGCACCGAGUCGGUGCUUUUUUU
(version 5; SEQ ID NO: 26);
GUUUUAGAGCUAGAAAUAGCAAGUUAAAAUAAGGCUAGUCCGUUAUCAA
CUUGAAAAAGUGGCACCGAGUCGGUGCUUUU
(version 6; scaffold A; SEQ ID NO: 27);
GUUUAAGAGCUAUGCUGGAAACAGCAUAGCAAGUUUAAAUAAGGCUAGU
CCGUUAUCAACUUGAAAAAGUGGCACCGAGUCGGUGCUUUUUU
(version 7; SEQ ID NO: 28);
or
GUUUUAGAGCUAGAAAUAGCAAGUUAAAAUAAGGCUAGUCCGUUAUCAA
CUUGGCACCGAGUCGGUGC
(version 8; SEQ ID NO: 29).

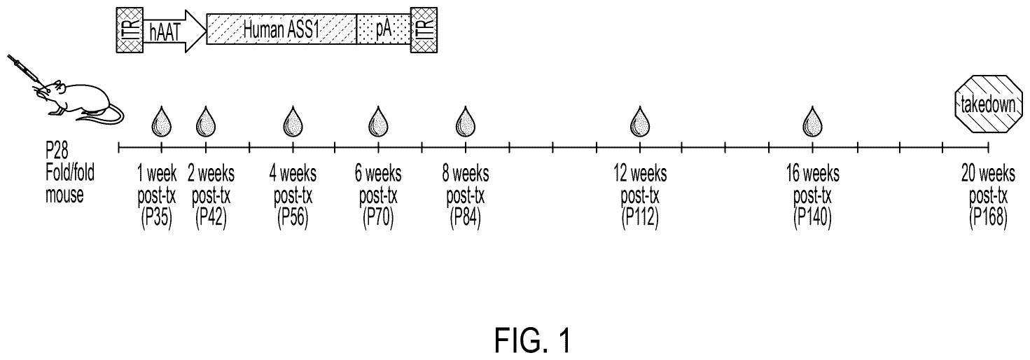

In some guide sgRNAs, the four terminal U residues of version 6 are not present. In some sgRNAs, only 1, 2, or 3 of the four terminal U residues of version 6 are present. In some sgRNAs, the scaffold of version 6 (with all four terminal U residues, or only 1, 2, or 3 terminal U residues (e.g., only 1 terminal U residue, like in scaffold version 1) or version 1 can be shortened. See, e.g., WO 2021/119275, herein incorporated by reference in its entirety for all purposes. For example, a hairpin region from scaffold version 6 or scaffold version 1 can be shortened by deleting several residues (e.g., 6 residues). As one example, hairpin residues H1-1, H1-3, H1-4, H1-9, H1-10, and H1-12 shown in FIG. 1A in WO 2021/119275 can be deleted. For example, another exemplary scaffold sequence (e.g., for use with S. pyogenes Cas9) comprises, consists essentially of, or consists of GUUUUAGAGCUAGAAAUAGCAAGUUAAAAUAAGGCUAGUCCGUUAUCACGAAAG GGCACCGAGUCGGUGCU (version 9; scaffold B; SEQ ID NO: 428). Other exemplary guide RNA scaffolds can be found in WO 2021/119275 and US 2022-0372483, each of which is herein incorporated by reference in its entirety for all purposes. Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). That is, guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage (an exemplary backbone modification); (2) alteration or replacement of a constituent of the ribose sugar such as alteration or replacement of the 2′ hydroxyl on the ribose sugar (an exemplary sugar modification); (3) replacement (e.g., wholesale replacement) of the phosphate moiety with dephospho linkers (an exemplary backbone modification); (4) modification or replacement of a naturally occurring nucleobase, including with a non-canonical nucleobase (an exemplary base modification); (5) replacement or modification of the ribose-phosphate backbone (an exemplary backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification); and (7) modification or replacement of the sugar (an exemplary sugar modification). Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

Chemical modifications such as those listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively, or additionally, a modified gRNA can comprise at least one modified residue at or near the 5′ end. Alternatively, or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

The gRNAs disclosed herein can comprise a backbone modification in which the phosphate group of a modified residue can be modified by replacing one or more of the oxygens with a different substituent. The modification can include the wholesale replacement of an unmodified phosphate moiety with a modified phosphate group as described herein. Backbone modifications of the phosphate backbone can also include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (Rp) or the “S” configuration (Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group (a sugar modification). For example, the 2′ hydroxyl group (OH) can be modified (e.g., replaced with a number of different oxy or deoxy substituents. Modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). The 2′ hydroxyl group modification can be 2′-O-Me. Likewise, the 2′ hydroxyl group modification can be a 2′-fluoro modification, which replaces the 2′ hydroxyl group with a fluoride. The 2′ hydroxyl group modification can include locked nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). The 2′ hydroxyl group modification can include unlocked nucleic acids (UNA) in which the ribose ring lacks the C2′-C3′ bond. The 2′ hydroxyl group modification can include the methoxyethyl group (MOE), (OCH₂CH₂OCH₃, e.g., a PEG derivative).

Deoxy 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH2; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form (e.g., L-nucleosides).

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification.

The guide RNAs disclosed herein can comprise one of the modification patterns disclosed in WO 2018/107028 A1, herein incorporated by reference in its entirety for all purposes. The guide RNAs disclosed herein can also comprise one of the structures/modification patterns disclosed in US 2017/0114334, herein incorporated by reference in its entirety for all purposes. The guide RNAs disclosed herein can also comprise one of the structures/modification patterns disclosed in WO 2017/136794, WO 2017/004279, US 2018/0187186, or US 2019/0048338, each of which is herein incorporated by reference in its entirety for all purposes.

As one example, nucleotides at the 5′ or 3′ end of a guide RNA can include phosphorothioate linkages (e.g., the bases can have a modified phosphate group that is a phosphorothioate group). For example, a guide RNA can include phosphorothioate linkages between the 2, 3, or 4 terminal nucleotides at the 5′ or 3′ end of the guide RNA. As another example, nucleotides at the 5′ and/or 3′ end of a guide RNA can have 2′-O-methyl modifications. For example, a guide RNA can include 2′-O-methyl modifications at the 2, 3, or 4 terminal nucleotides at the 5′ and/or 3′ end of the guide RNA (e.g., the 5′ end). See, e.g., WO 2017/173054 A1 and Finn et al. (2018) Cell Rep. 22(9):2227-2235, each of which is herein incorporated by reference in its entirety for all purposes. Other possible modifications are described in more detail elsewhere herein. In a specific example, a guide RNA includes 2′-O-methyl analogs and 3′ phosphorothioate internucleotide linkages at the first three 5′ and 3′ terminal RNA residues. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively, or additionally, the at least one modification can comprise a phosphorothioate (PS) bond between nucleotides. Alternatively, or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

The modifications can occur anywhere in the guide RNA. As one example, the guide RNA comprises a modification at one or more of the first five nucleotides at the 5′ end of the guide RNA, the guide RNA comprises a modification at one or more of the last five nucleotides of the 3′ end of the guide RNA, or a combination thereof. For example, the guide RNA can comprise phosphorothioate bonds between the first four nucleotides of the guide RNA, phosphorothioate bonds between the last four nucleotides of the guide RNA, or a combination thereof. Alternatively, or additionally, the guide RNA can comprise 2′-O-Me modified nucleotides at the first three nucleotides at the 5′ end of the guide RNA, can comprise 2′-O-Me modified nucleotides at the last three nucleotides at the 3′ end of the guide RNA, or a combination thereof.

In one example, a modified gRNA can comprise the following sequence: mN*mN*mN*NNNNNNNNNNNNNNNNNGUUUUAGAmGmCmUmAmGmAmAmAmUmA mGmCAAGUUAAAAUAAGGCUAGUCCGUUAUCAmAmCmUmUmGmAmAmAmAmAm GmUmGmGmCmAmCmCmGmAmGmUmCmGmGmUmGmCmU*mU*mU*mU (SEQ ID NO: 30), where “N” may be any natural or non-natural nucleotide. For example, the totality of N residues can comprise a human ASS1 intron 2 DNA-targeting segment as described herein (e.g., the sequence set forth in SEQ ID NO: 30, wherein the N residues are replaced with the DNA-targeting segment of any one of SEQ ID NOS: 31-118, the DNA-targeting segment of any one of SEQ ID NOS: 58, 60, 67, 73, 78, 89, 92, and 114, the DNA-targeting segment of SEQ ID NO: 58, or the DNA-targeting segment of SEQ ID NO: 67. For example, the totality of N residues can comprise a human ASS1 intron 2 DNA-targeting segment as described herein (e.g., the sequence set forth in SEQ ID NO: 30, wherein the N residues are replaced with the DNA-targeting segment of any one of SEQ ID NOS: 78, 60, and 89. For example, the totality of N residues can comprise a human ASS1 intron 2 DNA-targeting segment as described herein (e.g., the sequence set forth in SEQ ID NO: 30, wherein the N residues are replaced with the DNA-targeting segment of SEQ ID NOS: 78. For example, the totality of N residues can comprise a human ASS1 intron 2 DNA-targeting segment as described herein (e.g., the sequence set forth in SEQ ID NO: 30, wherein the N residues are replaced with the DNA-targeting segment of SEQ ID NOS: 60. For example, the totality of N residues can comprise a human ASS1 intron 2 DNA-targeting segment as described herein (e.g., the sequence set forth in SEQ ID NO: 30, wherein the N residues are replaced with the DNA-targeting segment of SEQ ID NOS: 89. Likewise, for example, the totality of N residues can comprise a human ASS1 intron 1 DNA-targeting segment as described herein (e.g., the sequence set forth in SEQ ID NO: 30, wherein the N residues are replaced with the DNA-targeting segment of any one of SEQ ID NOS: 430-517. Likewise, for example, the totality of N residues can comprise a mouse ASS1 intron 1 DNA-targeting segment as described herein (e.g., the sequence set forth in SEQ ID NO: 30, wherein the N residues are replaced with the DNA-targeting segment of any one of SEQ ID NOS: 207-212. The terms “mA,” “mC,” “mU,” and “mG” denote a nucleotide (A, C, U, and G, respectively) that has been modified with 2′-O-Me. The symbol “*” depicts a phosphorothioate modification. A phosphorothioate linkage or bond refers to a bond where a sulfur is substituted for one nonbridging phosphate oxygen in a phosphodiester linkage, for example, in the bonds between nucleotides bases. When phosphorothioates are used to generate oligonucleotides, the modified oligonucleotides may also be referred to as S-oligos. The terms A*, C*, U*, or G* denote a nucleotide that is linked to the next (e.g., 3′) nucleotide with a phosphorothioate bond. The terms “mA*,” “mC*,” “mU*,” and “mG*” denote a nucleotide (A, C, U, and G, respectively) that has been substituted with 2′-O-Me and that is linked to the next (e.g., 3′) nucleotide with a phosphorothioate bond.

In another example, a modified gRNA can comprise the following sequence: mN*mN*mN*NNNNNNNNNNNNNNNNNGUUUUAGAmGmCmUmAmGmAmAmAmUmA mGmCAAGUUAAAAUAAGGCUAGUCCGUUAUCACGAAAGGGCACCGAGUCGGmU*mG*mC*mU (SEQ ID NO: 429), where “N” may be any natural or non-natural nucleotide. For example, the totality of N residues can comprise a human ASS1 intron 2 DNA-targeting segment as described herein (e.g., the sequence set forth in SEQ ID NO: 429, wherein the N residues are replaced with the DNA-targeting segment of any one of SEQ ID NOS: 31-118, the DNA-targeting segment of any one of SEQ ID NOS: 58, 60, 67, 73, 78, 89, 92, and 114, the DNA-targeting segment of SEQ ID NO: 58, or the DNA-targeting segment of SEQ ID NO: 67. For example, the totality of N residues can comprise a human ASS1 intron 2 DNA-targeting segment as described herein (e.g., the sequence set forth in SEQ ID NO: 429, wherein the N residues are replaced with the DNA-targeting segment of any one of SEQ ID NOS: 78, 60, and 89. For example, the totality of N residues can comprise a human ASS1 intron 2 DNA-targeting segment as described herein (e.g., the sequence set forth in SEQ ID NO: 429, wherein the N residues are replaced with the DNA-targeting segment of SEQ ID NO: 78. For example, the totality of N residues can comprise a human ASS1 intron 2 DNA-targeting segment as described herein (e.g., the sequence set forth in SEQ ID NO: 429, wherein the N residues are replaced with the DNA-targeting segment of SEQ ID NO: 60. For example, the totality of N residues can comprise a human ASS1 intron 2 DNA-targeting segment as described herein (e.g., the sequence set forth in SEQ ID NO: 429, wherein the N residues are replaced with the DNA-targeting segment of SEQ ID NO: 89. Likewise, for example, the totality of N residues can comprise a human ASS1 intron 1 DNA-targeting segment as described herein (e.g., the sequence set forth in SEQ ID NO: 429, wherein the N residues are replaced with the DNA-targeting segment of any one of SEQ ID NOS: 430-517. Likewise, for example, the totality of N residues can comprise a mouse ASS1 intron 1 DNA-targeting segment as described herein (e.g., the sequence set forth in SEQ ID NO: 429, wherein the N residues are replaced with the DNA-targeting segment of any one of SEQ ID NOS: 207-212. The terms “mA,” “mC,” “mU,” and “mG” denote a nucleotide (A, C, U, and G, respectively) that has been modified with 2′-O-Me. The symbol “*” depicts a phosphorothioate modification. A phosphorothioate linkage or bond refers to a bond where a sulfur is substituted for one nonbridging phosphate oxygen in a phosphodiester linkage, for example, in the bonds between nucleotides bases. When phosphorothioates are used to generate oligonucleotides, the modified oligonucleotides may also be referred to as S-oligos. The terms A*, C*, U*, or G* denote a nucleotide that is linked to the next (e.g., 3′) nucleotide with a phosphorothioate bond. The terms “mA*,” “mC*,” “mU*,” and “mG*” denote a nucleotide (A, C, U, and G, respectively) that has been substituted with 2′-O-Me and that is linked to the next (e.g., 3′) nucleotide with a phosphorothioate bond.

Other exemplary modified gRNAs can be found in WO 2021/119275 and US 2022-0372483, each of which is herein incorporated by reference in its entirety for all purposes.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Abasic nucleotides refer to those which lack nitrogenous bases. Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In one example, one or more of the first three, four, or five nucleotides at the 5′ terminus, and one or more of the last three, four, or five nucleotides at the 3′ terminus are modified. The modification can be, for example, a 2′-O-Me, 2′-F, inverted abasic nucleotide, phosphorothioate bond, or other nucleotide modification well known to increase stability and/or performance.

In another example, the first four nucleotides at the 5′ terminus, and the last four nucleotides at the 3′ terminus can be linked with phosphorothioate bonds.

In another example, the first three nucleotides at the 5′ terminus, and the last three nucleotides at the 3′ terminus can comprise a 2′-O-methyl (2′-O-Me) modified nucleotide. In another example, the first three nucleotides at the 5′ terminus, and the last three nucleotides at the 3′ terminus comprise a 2′-fluoro (2′-F) modified nucleotide. In another example, the first three nucleotides at the 5′ terminus, and the last three nucleotides at the 3′ terminus comprise an inverted abasic nucleotide.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as one DNA molecule or as separate DNA molecules encoding the crRNA and tracrRNA, respectively.

When a gRNA is provided in the form of DNA, the gRNA can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid, such as a nucleic acid encoding a Cas protein. Alternatively, it can be in a vector or a plasmid that is separate from the vector comprising the nucleic acid encoding the Cas protein. Promoters that can be used in such expression constructs include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis. For example, a guide RNA can be chemically synthesized to include 2′-O-methyl analogs and 3′ phosphorothioate internucleotide linkages at the first three 5′ and 3′ terminal RNA residues.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

As one example, a guide RNA targeting intron 2 of a human ASS1 gene can comprise, consist essentially of, or consist of the sequence set forth in any one of SEQ ID NOS: 252-339. Alternatively, a guide RNA targeting intron 2 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in any one of SEQ ID NOS: 252-339. Alternatively, a guide RNA targeting intron 2 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in any one of SEQ ID NOS: 252-339. Alternatively, a guide RNA targeting intron 2 of a human ASS1 gene can comprise, consist essentially of, or consist of a sequence that differs by no more than 3, no more than 2, or no more than 1 nucleotide from the sequence set forth in any one of SEQ ID NOS: 252-339.

As one example, a guide RNA targeting intron 1 of a mouse Ass1 gene can comprise, consist essentially of, or consist of the sequence set forth in any one of SEQ ID NOS: 219-224. Alternatively, a guide RNA targeting intron 1 of a mouse Ass1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in any one of SEQ ID NOS: 219-224. Alternatively, a guide RNA targeting intron 1 of a mouse Ass1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in any one of SEQ ID NOS: 219-224. Alternatively, a guide RNA targeting intron 1 of a mouse Ass1 gene can comprise, consist essentially of, or consist of a sequence that differs by no more than 3, no more than 2, or no more than 1 nucleotide from the sequence set forth in any one of SEQ ID NOS: 219-224.

As one example, a guide RNA targeting intron 1 of a mouse Ass1 gene can comprise, consist essentially of, or consist of the sequence set forth in any one of SEQ ID NOS: 697-702. Alternatively, a guide RNA targeting intron 1 of a mouse Ass1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in any one of SEQ ID NOS: 697-702. Alternatively, a guide RNA targeting intron 1 of a mouse Ass1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in any one of SEQ ID NOS: 697-702. Alternatively, a guide RNA targeting intron 1 of a mouse Ass1 gene can comprise, consist essentially of, or consist of a sequence that differs by no more than 3, no more than 2, or no more than 1 nucleotide from the sequence set forth in any one of SEQ ID NOS: 697-702.

As another example, a guide RNA targeting intron 2 of a human ASS1 gene can comprise, consist essentially of, or consist of the sequence set forth in any one of SEQ ID NOS: 279, 281, 288, 294, 299, 310, 313, and 335. Alternatively, a guide RNA targeting intron 2 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in any one of SEQ ID NOS: 279, 281, 288, 294, 299, 310, 313, and 335. Alternatively, a guide RNA targeting intron 2 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in any one of SEQ ID NOS: 279, 281, 288, 294, 299, 310, 313, and 335. Alternatively, a guide RNA targeting intron 2 of a human ASS1 gene can comprise, consist essentially of, or consist of a sequence that differs by no more than 3, no more than 2, or no more than 1 nucleotide from the sequence set forth in any one of SEQ ID NOS: 279, 281, 288, 294, 299, 310, 313, and 335.

As another example, a guide RNA targeting intron 2 of a human ASS1 gene can comprise, consist essentially of, or consist of the sequence set forth in any one of SEQ ID NOS: 299, 281, and 310. Alternatively, a guide RNA targeting intron 2 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in any one of SEQ ID NOS: 299, 281, and 310. Alternatively, a guide RNA targeting intron 2 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in any one of SEQ ID NOS: 299, 281, and 310. Alternatively, a guide RNA targeting intron 2 of a human ASS1 gene can comprise, consist essentially of, or consist of a sequence that differs by no more than 3, no more than 2, or no more than 1 nucleotide from the sequence set forth in any one of SEQ ID NOS: 299, 281, and 310.

As another example, a guide RNA targeting intron 2 of a human ASS1 gene can comprise, consist essentially of, or consist of the sequence set forth in SEQ ID NO: 299. Alternatively, a guide RNA targeting intron 2 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 299. Alternatively, a guide RNA targeting intron 2 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in SEQ ID NO: 299. Alternatively, a guide RNA targeting intron 2 of a human ASS1 gene can comprise, consist essentially of, or consist of a sequence that differs by no more than 3, no more than 2, or no more than 1 nucleotide from the sequence set forth in SEQ ID NO: 299.

As another example, a guide RNA targeting intron 2 of a human ASS1 gene can comprise, consist essentially of, or consist of the sequence set forth in SEQ ID NO: 281. Alternatively, a guide RNA targeting intron 2 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 281. Alternatively, a guide RNA targeting intron 2 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in SEQ ID NO: 281. Alternatively, a guide RNA targeting intron 2 of a human ASS1 gene can comprise, consist essentially of, or consist of a sequence that differs by no more than 3, no more than 2, or no more than 1 nucleotide from the sequence set forth in SEQ ID NO: 281.

As another example, a guide RNA targeting intron 2 of a human ASS1 gene can comprise, consist essentially of, or consist of the sequence set forth in SEQ ID NO: 310. Alternatively, a guide RNA targeting intron 2 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 310. Alternatively, a guide RNA targeting intron 2 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in SEQ ID NO: 310. Alternatively, a guide RNA targeting intron 2 of a human ASS1 gene can comprise, consist essentially of, or consist of a sequence that differs by no more than 3, no more than 2, or no more than 1 nucleotide from the sequence set forth in SEQ ID NO: 310.

As another example, a guide RNA targeting intron 2 of a human ASS1 gene can comprise, consist essentially of, or consist of the sequence set forth in SEQ ID NO: 279. Alternatively, a guide RNA targeting intron 2 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 279. Alternatively, a guide RNA targeting intron 2 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in SEQ ID NO: 279. Alternatively, a guide RNA targeting intron 2 of a human ASS1 gene can comprise, consist essentially of, or consist of a sequence that differs by no more than 3, no more than 2, or no more than 1 nucleotide from the sequence set forth in SEQ ID NO: 279.

As another example, a guide RNA targeting intron 2 of a human ASS1 gene can comprise, consist essentially of, or consist of the sequence set forth in SEQ ID NO: 288. Alternatively, a guide RNA targeting intron 2 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 288. Alternatively, a guide RNA targeting intron 2 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in SEQ ID NO: 288. Alternatively, a guide RNA targeting intron 2 of a human ASS1 gene can comprise, consist essentially of, or consist of a sequence that differs by no more than 3, no more than 2, or no more than 1 nucleotide from the sequence set forth in SEQ ID NO: 288.

As one example, a guide RNA targeting intron 2 of a human ASS1 gene can comprise, consist essentially of, or consist of the sequence set forth in any one of SEQ ID NOS: 340-427. Alternatively, a guide RNA targeting intron 2 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in any one of SEQ ID NOS: 340-427. Alternatively, a guide RNA targeting intron 2 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in any one of SEQ ID NOS: 340-427. Alternatively, a guide RNA targeting intron 2 of a human ASS1 gene can comprise, consist essentially of, or consist of a sequence that differs by no more than 3, no more than 2, or no more than 1 nucleotide from the sequence set forth in any one of SEQ ID NOS: 340-427.

As one example, a guide RNA targeting intron 2 of a cynomolgus macaque ASS1 gene can comprise, consist essentially of, or consist of the sequence set forth in SEQ ID NO: 716. Alternatively, a guide RNA targeting intron 2 of a cynomolgus macaque ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 716. Alternatively, a guide RNA targeting intron 2 of a cynomolgus macaque ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in SEQ ID NO: 716. Alternatively, a guide RNA targeting intron 2 of a cynomolgus macaque ASS1 gene can comprise, consist essentially of, or consist of a sequence that differs by no more than 3, no more than 2, or no more than 1 nucleotide from the sequence set forth in SEQ ID NO: 716.

As another example, a guide RNA targeting intron 2 of a human ASS1 gene can comprise, consist essentially of, or consist of the sequence set forth in any one of SEQ ID NOS: 367, 369, 376, 382, 387, 398, 401, and 423. Alternatively, a guide RNA targeting intron 2 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in any one of SEQ ID NOS: 367, 369, 376, 382, 387, 398, 401, and 423. Alternatively, a guide RNA targeting intron 2 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in any one of SEQ ID NOS: 367, 369, 376, 382, 387, 398, 401, and 423. Alternatively, a guide RNA targeting intron 2 of a human ASS1 gene can comprise, consist essentially of, or consist of a sequence that differs by no more than 3, no more than 2, or no more than 1 nucleotide from the sequence set forth in any one of SEQ ID NOS: 367, 369, 376, 382, 387, 398, 401, and 423.

As another example, a guide RNA targeting intron 2 of a human ASS1 gene can comprise, consist essentially of, or consist of the sequence set forth in any one of SEQ ID NOS: 387, 369, and 398. Alternatively, a guide RNA targeting intron 2 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in any one of SEQ ID NOS: 387, 369, and 398. Alternatively, a guide RNA targeting intron 2 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in any one of SEQ ID NOS: 387, 369, and 398. Alternatively, a guide RNA targeting intron 2 of a human ASS1 gene can comprise, consist essentially of, or consist of a sequence that differs by no more than 3, no more than 2, or no more than 1 nucleotide from the sequence set forth in any one of SEQ ID NOS: 387, 369, and 398.

As another example, a guide RNA targeting intron 2 of a human ASS1 gene can comprise, consist essentially of, or consist of the sequence set forth in SEQ ID NO: 387. Alternatively, a guide RNA targeting intron 2 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 387. Alternatively, a guide RNA targeting intron 2 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in SEQ ID NO: 387. Alternatively, a guide RNA targeting intron 2 of a human ASS1 gene can comprise, consist essentially of, or consist of a sequence that differs by no more than 3, no more than 2, or no more than 1 nucleotide from the sequence set forth in SEQ ID NO: 387.

As another example, a guide RNA targeting intron 2 of a human ASS1 gene can comprise, consist essentially of, or consist of the sequence set forth in SEQ ID NO: 369. Alternatively, a guide RNA targeting intron 2 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 369. Alternatively, a guide RNA targeting intron 2 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in SEQ ID NO: 369. Alternatively, a guide RNA targeting intron 2 of a human ASS1 gene can comprise, consist essentially of, or consist of a sequence that differs by no more than 3, no more than 2, or no more than 1 nucleotide from the sequence set forth in SEQ ID NO: 369.

As another example, a guide RNA targeting intron 2 of a human ASS1 gene can comprise, consist essentially of, or consist of the sequence set forth in SEQ ID NO: 398. Alternatively, a guide RNA targeting intron 2 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 398. Alternatively, a guide RNA targeting intron 2 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in SEQ ID NO: 398. Alternatively, a guide RNA targeting intron 2 of a human ASS1 gene can comprise, consist essentially of, or consist of a sequence that differs by no more than 3, no more than 2, or no more than 1 nucleotide from the sequence set forth in SEQ ID NO: 398.

As another example, a guide RNA targeting intron 2 of a human ASS1 gene can comprise, consist essentially of, or consist of the sequence set forth in SEQ ID NO: 367. Alternatively, a guide RNA targeting intron 2 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 367. Alternatively, a guide RNA targeting intron 2 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in SEQ ID NO: 367. Alternatively, a guide RNA targeting intron 2 of a human ASS1 gene can comprise, consist essentially of, or consist of a sequence that differs by no more than 3, no more than 2, or no more than 1 nucleotide from the sequence set forth in SEQ ID NO: 367.

As another example, a guide RNA targeting intron 2 of a human ASS1 gene can comprise, consist essentially of, or consist of the sequence set forth in SEQ ID NO: 376. Alternatively, a guide RNA targeting intron 2 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 376. Alternatively, a guide RNA targeting intron 2 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in SEQ ID NO: 376. Alternatively, a guide RNA targeting intron 2 of a human ASS1 gene can comprise, consist essentially of, or consist of a sequence that differs by no more than 3, no more than 2, or no more than 1 nucleotide from the sequence set forth in SEQ ID NO: 376.

As one example, a guide RNA targeting intron 1 of a human ASS1 gene can comprise, consist essentially of, or consist of the sequence set forth in any one of SEQ ID NOS: 606-693. Alternatively, a guide RNA targeting intron 1 of a human ASS1 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the sequence set forth in any one of SEQ ID NOS: 606-693. Alternatively, a guide RNA targeting intron 1 of a human ASS1 gene can comprise, consist essentially of, or consist of a sequence that is at least 90%, at least 91%, at least 92%, at least 93%, at least 94%, at least 95%, at least 96%, at least 97%, at least 98%, or at least 99% identical to the sequence set forth in any one of SEQ ID NOS: 606-693. Alternatively, a guide RNA targeting intron 1 of a human ASS1 gene can comprise, consist essentially of, or consist of a sequence that differs by no more than 3, no more than 2, or no more than 1 nucleotide from the sequence set forth in any one of SEQ ID NOS: 606-693.

### (4) Guide RNA Target Sequences

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3^{rd} Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand.”

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g., 3 base pairs) upstream or downstream of the PAM sequence (e.g., within the guide RNA target sequence). In the case of SpCas9, the PAM sequence (i.e., on the non-complementary strand) can be 5′-N₁GG-3′, where N₁ is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN₂-3′, where N₂ is any DNA nucleotide and is immediately 5′ of the sequence to which the DNA-targeting segment of the guide RNA hybridizes on the complementary strand of the target DNA. In some such cases, N₁ and N₂ can be complementary and the N₁-N₂ base pair can be any base pair (e.g., N₁=C and N₂=G; N₁=G and N₂=C; N₁=A and N₂=T; or N₁=T, and N₂=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3′. In the case of DpbCasX, the PAM can have the sequence 5′-TTCN-3′. In the case of CasΦ, the PAM can have the sequence 5′-TBN-3′, wherein B is G, T, or C.

An example of a guide RNA target sequence is a 20-nucleotide DNA sequence immediately preceding an NGG motif recognized by an SpCas9 protein. For example, two examples of guide RNA target sequences plus PAMs are GN₁₉NGG (SEQ ID NO: 6) or N₂₀NGG (SEQ ID NO: 7). See, e.g., WO 2014/165825, herein incorporated by reference in its entirety for all purposes. The guanine at the 5′ end can facilitate transcription by RNA polymerase in cells. Other examples of guide RNA target sequences plus PAMs can include two guanine nucleotides at the 5′ end (e.g., GGN₂₀NGG; SEQ ID NO: 8) to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length of SEQ ID NOS: 6-8, including the 5′ G or GG and the 3′ GG or NGG. Yet other guide RNA target sequences plus PAMs can have between 14 and 20 nucleotides in length of SEQ ID NOS: 6-8.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g., Cas9)) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75, 100, 250, 500, or 1,000 base pairs.

The guide RNA target sequence can also be selected to minimize off-target modification or avoid off-target effects (e.g., by avoiding two or fewer mismatches to off-target genomic sequences).

As one example, a guide RNA targeting intron 2 of a human ASS1 gene can target the guide RNA target sequence set forth in any one of SEQ ID NOS: 119-206. As another example, a guide RNA targeting intron 2 of a human ASS1 gene can target at least 17, at least 18, at least 19, or at least 20 contiguous nucleotides of the guide RNA target sequence set forth in any one of SEQ ID NOS: 119-206.

As one example, a guide RNA targeting intron 2 of a cynomolgus macaque ASS1 gene can target the guide RNA target sequence set forth in SEQ ID NO: 715. As another example, a guide RNA targeting intron 2 of a cynomolgus macaque ASS1 gene can target at least 17, at least 18, at least 19, or at least 20 contiguous nucleotides of the guide RNA target sequence set forth in SEQ ID NO: 715.

As one example, a guide RNA targeting intron 1 of a human ASS1 gene can target the guide RNA target sequence set forth in any one of SEQ ID NOS: 518-605. As another example, a guide RNA targeting intron 1 of a human ASS1 gene can target at least 17, at least 18, at least 19, or at least 20 contiguous nucleotides of the guide RNA target sequence set forth in any one of SEQ ID NOS: 518-605.

As one example, a guide RNA targeting intron 1 of a mouse Ass1 gene can target the guide RNA target sequence set forth in any one of SEQ ID NOS: 213-218. As another example, a guide RNA targeting intron 1 of a mouse Ass1 gene can target at least 17, at least 18, at least 19, or at least 20 contiguous nucleotides of the guide RNA target sequence set forth in any one of SEQ ID NOS: 213-218.

As another example, a guide RNA targeting intron 2 of a human ASS1 gene can target the guide RNA target sequence set forth in any one of SEQ ID NOS: 146, 148, 155, 161, 166, 177, 180, and 202. As another example, a guide RNA targeting intron 2 of a human ASS1 gene can target at least 17, at least 18, at least 19, or at least 20 contiguous nucleotides of the guide RNA target sequence set forth in any one of SEQ ID NOS: 146, 148, 155, 161, 166, 177, 180, and 202.

As another example, a guide RNA targeting intron 2 of a human ASS1 gene can target the guide RNA target sequence set forth in any one of SEQ ID NOS: 166, 148, and 177. As another example, a guide RNA targeting intron 2 of a human ASS1 gene can target at least 17, at least 18, at least 19, or at least 20 contiguous nucleotides of the guide RNA target sequence set forth in any one of SEQ ID NOS: 166, 148, and 177.

As another example, a guide RNA targeting intron 2 of a human ASS1 gene can target the guide RNA target sequence set forth in SEQ ID NO: 166. As another example, a guide RNA targeting intron 2 of a human ASS1 gene can target at least 17, at least 18, at least 19, or at least 20 contiguous nucleotides of the guide RNA target sequence set forth in SEQ ID NO: 166.

As another example, a guide RNA targeting intron 2 of a human ASS1 gene can target the guide RNA target sequence set forth in SEQ ID NO: 148. As another example, a guide RNA targeting intron 2 of a human ASS1 gene can target at least 17, at least 18, at least 19, or at least 20 contiguous nucleotides of the guide RNA target sequence set forth in SEQ ID NO: 148.

As another example, a guide RNA targeting intron 2 of a human ASS1 gene can target the guide RNA target sequence set forth in SEQ ID NO: 177. As another example, a guide RNA targeting intron 2 of a human ASS1 gene can target at least 17, at least 18, at least 19, or at least 20 contiguous nucleotides of the guide RNA target sequence set forth in SEQ ID NO: 177.

As another example, a guide RNA targeting intron 2 of a human ASS1 gene can target the guide RNA target sequence set forth in SEQ ID NO: 146. As another example, a guide RNA targeting intron 2 of a human ASS1 gene can target at least 17, at least 18, at least 19, or at least 20 contiguous nucleotides of the guide RNA target sequence set forth in SEQ ID NO: 146.

As another example, a guide RNA targeting intron 2 of a human ASS1 gene can target the guide RNA target sequence set forth in SEQ ID NO: 155. As another example, a guide RNA targeting intron 2 of a human ASS1 gene can target at least 17, at least 18, at least 19, or at least 20 contiguous nucleotides of the guide RNA target sequence set forth in SEQ ID NO: 155.

TABLE 10
Human ASSI Intron 2 Guide RNA Target Sequences.

| Guide | | Guide RNA | | SEQ |
|---|---|---|---|---|
| RNA ID# | Strand | Target Sequence | PAM | ID NO: |
| G035811 | + | GAGTGCGCTTTCAGCAGCGC | AGG | 119 |
| G035812 | + | CCTCTACACTCTAATTTACG | TGG | 120 |
| G035813 | + | CAGCGGGGGTGGCCCCGTAT | AGG | 121 |
| G035814 | − | AGCTAGCTGAGACCTATACG | GGG | 122 |
| G035815 | − | CCACGTAAATTAGAGTGTAG | AGG | 123 |
| G035816 | − | CACTCGAATCTTCAACACTC | AGG | 124 |
| G035817 | − | ACTCGAATCTTCAACACTCA | GGG | 125 |
| G035818 | + | GTTTCACAGAACGGTGCATC | AGG | 126 |
| G035819 | − | GGTTGCCACAACTACCAGGT | GGG | 127 |
| G035820 | + | TGGCCTTGTTCCCGTGTGGC | TGG | 128 |
| G035821 | 1 | CGGCCAGCCACACGGGAACA | AGG | 129 |
| G035822 | − | AAACAAGGCATCACATAGCG | AGG | 130 |
| G035823 | + | GTATAGGTCTCAGCTAGCTG | AGG | 131 |
| G035824 | + | GGTGCCTTGATTGTGAATCT | AGG | 132 |
| G035825 | + | GTGCATCAGGATTGATTAGC | AGG | 133 |
| G035826 | − | GGGGTTCCCAATGACTTTTC | AGG | 134 |
| G035827 | − | TCAGCTAGCTGAGACCTATA | CGG | 135 |
| G035828 | − | GATGCACCGTTCTGTGAAAC | TGG | 136 |
| G035829 | − | GACGGTTTTGTCCCTGTTGG | GGG | 137 |
| G035830 | − | CTTGAAATGTAGACCCCCAG | GGG | 138 |
| G035831 | + | AAGAAGCACCAGGTACAGCG | GGG | 139 |
| G035832 | − | TTGCCACAACTACCAGGTGG | GGG | 140 |
| G035833 | − | TTCTTGAAATGTAGACCCCC | AGG | 141 |
| G035834 | + | AGCACCAGGTACAGCGGGGG | TGG | 142 |
| G035835 | + | TTATGATGAACAGTGTCATC | AGG | 143 |
| G035836 | + | CATGTTAAAATGAACACGGG | AGG | 144 |
| G035837 | − | GAAGAGACACTCCACCTGGA | TGG | 145 |
| G035838 | − | TCTGCTCTCCAGCACTCGGG | AGG | 146 |
| G035839 | + | GTCTGAGCAGTAGTCCAGGA | AGG | 147 |
| G035840 | − | ATCTGGCATACAGAGCCCCA | GGG | 148 |
| G035841 | − | CACAACTACCAGGTGGGGGG | TGG | 149 |
| G035842 | + | AGAGTAACAGTGCAGTGTGC | TGG | 150 |
| G035843 | − | TTTAAGAGTCGAGAAATTCT | TGG | 151 |
| G035844 | − | GTTTTGTCCCTGTTGGGGGT | GGG | 152 |
| G035845 | + | GTGGCTGGCCGTCCTGTGCA | CGG | 153 |
| G035846 | − | CATCTGGCATACAGAGCCCC | AGG | 154 |
| G035847 | − | CTGTGGCCAGTAGGTGACTT | GGG | 155 |
| G035848 | + | TCTTGCATATAACATTAGGA | TGG | 156 |
| G035849 | − | ATGAAGGGGCACAAGTGGCA | AGG | 157 |
| G035850 | + | AAAAGTCATTGGGAACCCCT | GGG | 158 |
| G035851 | − | GTGGAGGGATTAATGACAGA | TGG | 159 |
| G035852 | − | CTGCAGAAGCACCCCACAGA | GGG | 160 |
| G035853 | − | AGGCAAGGACAGGGGCCATC | TGG | 161 |
| G035854 | − | TCTGGCATACAGAGCCCCAG | GGG | 162 |
| G035855 | − | GAAGAAGTAGGCAAGGACAG | GGG | 163 |
| G035856 | + | AGAACCATTCCTCTGGATCG | GGG | 164 |
| G035857 | − | CAGCTAGCTGAGACCTATAC | GGG | 165 |
| G035858 | + | GGTTGTCTGAGCAGTAGTCC | AGG | 166 |
| G035859 | − | GAAACCCCGATCCAGAGGAA | TGG | 167 |
| G035860 | − | TCACATAGCGAGGAAGCCCT | GGG | 168 |
| G035861 | + | CAGAGAACTCCAATTGAGTC | TGG | 169 |
| G035862 | + | GGGACAAAACCGTCTCTGGT | TGG | 170 |
| G035863 | − | ATCACATAGCGAGGAAGCCC | TGG | 171 |
| G035864 | − | ATAAATGCCCTGTATGGGAC | AGG | 172 |
| G035865 | − | AGACGGTTTTGTCCCTGTTG | GGG | 173 |
| G035866 | − | AAATGCCCTGTATGGGACAG | GGG | 174 |
| G035867 | + | TGATTGTGAATCTAGGCATG | TGG | 175 |
| G035868 | − | GAGACGGTTTTGTCCCTGTT | GGG | 176 |
| G035869 | − | ACTGAGAAACCCCGATCCAG | AGG | 177 |
| G035870 | − | CATAGCGAGGAAGCCCTGGG | AGG | 178 |
| G035871 | − | ACATTCTAGTTCCAGGCCTG | TGG | 179 |
| G035872 | − | ACTCGGGAGGCACAGAACCA | AGG | 180 |
| G035873 | − | CATGCCTAGATTCACAATCA | AGG | 181 |
| G035874 | + | GGAACTAGAATGTCCCTCCT | TGG | 182 |
| G035875 | + | CTCAGCTAGCTGAGGCCCCT | GGG | 183 |
| G035876 | − | AAGAGACACTCCACCTGGAT | GGG | 184 |
| G035877 | + | GAAAAGTCATTGGGAACCCC | TGG | 185 |
| G035878 | − | GCCAGTAGGTGACTTGGGAC | TGG | 186 |
| G035879 | − | ATCAAGGCACCAGACTCAAT | TGG | 187 |
| G035880 | + | ATCAGGATTGATTAGCAGGC | TGG | 188 |
| G035881 | − | TGCCACAACTACCAGGTGGG | GGG | 189 |
| G035882 | − | GACACTCCACCTGGATGGGA | AGG | 190 |
| G035883 | + | GCAGACCCCTGTCCCATACA | GGG | 191 |
| G035884 | − | GGGATCAGCTCTGACCCTGC | AGG | 192 |
| G035885 | + | TCAGCTAGCTGAGGCCCCTG | GGG | 193 |
| G035886 | + | CTGGGGCTCTGTATGCCAGA | TGG | 194 |
| G035887 | + | GAGTAACAGTGCAGTGTGCT | GGG | 195 |
| G035888 | + | GTGCTTCTGCAGGGTTGCCA | GGG | 196 |
| G035889 | + | TCAGAGCTGATCCCCTCTGT | GGG | 197 |
| G035890 | − | CGGGAGGCACAGAACCAAGG | AGG | 198 |
| G035891 | + | TGTCTCCCTGAAAAGTCATT | GGG | 199 |
| G035892 | + | AAAGTCATTGGGAACCCCTG | GGG | 200 |
| G035893 | + | GTGGTGGATGCTCCTGCCCC | AGG | 201 |
| G035894 | − | GTGGAATTCTGGCATCTAGT | GGG | 202 |
| G035895 | − | CCTGTGGCCAGTAGGTGACT | TGG | 203 |
| G035896 | − | ATGGATAGAAAAACGGTGAA | TGG | 204 |
| G035897 | − | CTCCACCTGGATGGGAAGGT | GGG | 205 |
| G035898 | − | ACTCCACCTGGATGGGAAGG | TGG | 206 |

TABLE 11
Human ASS1 Intron 1 Guide RNA Target Sequences.

| Guide | | Guide RNA | | SEQ |
|---|---|---|---|---|
| RNA ID# | Strand | Target Sequence | PAM | ID NO: |
| G033085 | − | CATAGGGACTAATGCGTGGT | GGG | 518 |
| G033086 | + | CACCGTCACTCATTCAAGCC | TGG | 519 |
| G033087 | − | GTGGCCAGGTTCAGTCGAAG | AGG | 520 |
| G033088 | − | AATGCGTGGTGGGTCCCCAT | GGG | 521 |
| G033089 | + | AGCTCCTCTTCGACTGAACC | TGG | 522 |
| G033090 | − | CCATAGGGACTAATGCGTGG | TGG | 523 |
| G033091 | − | CAGCCATAGGGACTAATGCG | TGG | 524 |
| G033092 | − | TCATTCGGCTCACCAGACCC | TGG | 525 |
| G033093 | + | GGTCTGGTGAGCCGAATGAA | GGG | 526 |
| G033094 | + | TGGCTTGTTTGCCGGCATAT | TGG | 527 |
| G033095 | + | AGGAGGGCTTACCCCCAGTA | GGG | 528 |
| G033096 | − | GCCGGCAAACAAGCCACTTT | TGG | 529 |
| G033097 | + | GAGCTGGGCGTATAGACCCC | TGG | 530 |
| G033098 | + | GCATCATGGGGTGCGAGGCT | TGG | 531 |
| G033099 | + | AGCTGGGCGTATAGACCCCT | GGG | 532 |
| G033100 | − | TAATGCGTGGTGGGTCCCCA | TGG | 533 |
| G033101 | + | AAGGAGGGCTTACCCCCAGT | AGG | 534 |
| G033102 | + | CATCATGGGGTGCGAGGCTT | GGG | 535 |
| G033103 | + | TCTCGAAGCCTGTCTTGAAC | TGG | 536 |
| G033104 | − | TTGGGAGCTGGATTGTAGCG | TGG | 537 |
| G033105 | + | GGGTCTGGTGAGCCGAATGA | AGG | 538 |
| G033106 | + | AGTCCCTATGGCTGCATCAT | GGG | 539 |
| G033107 | − | TGGGGGTAAGCCCTCCTTTT | TGG | 540 |
| G033108 | + | TTACCCCCAGTAGGGCCCAT | GGG | 541 |
| G033109 | − | TTGAGAGGGATGACTCCTAT | GGG | 542 |
| G033110 | − | GGGGGTAAGCCCTCCTTTTT | GGG | 543 |
| G033111 | + | TGACAAGCTAATCTTTGCAC | TGG | 544 |
| G033112 | + | GCCCAATGCCCTTGTCATTC | AGG | 545 |
| G033113 | + | TACCCCCAGTAGGGCCCATG | GGG | 546 |
| G033114 | − | CCTAGTCAAAGGCCATGCCT | TGG | 547 |
| G033115 | + | CACAGCGGGGCAATCAGAGC | TGG | 548 |
| G033116 | + | TAGACCCCTGGGCTACCACC | TGG | 549 |
| G033117 | − | ATCCCGCCCACTGAAGGAGT | AGG | 550 |
| G033118 | − | TGCAGGCTGACAGCATACTC | TGG | 551 |
| G033119 | − | CAGAACCAGGTGATCCCCCA | GGG | 552 |
| G033120 | + | GGGTGTATCCATCTCACTGT | AGG | 553 |
| G033121 | − | GGGTCCCCATGGGCCCTACT | GGG | 554 |
| G033122 | + | CAAGGCATGGCCTTTGACTA | GGG | 555 |
| G033123 | + | GATCTATCTCCAACTCTACT | TGG | 556 |
| G033124 | − | CAGTGAGATGGATACACCCT | GGG | 557 |
| G033125 | − | GAAGAAATCCCGCCCACTGA | AGG | 558 |
| G033126 | − | ATCCCCCAGGTGGTAGCCCA | GGG | 559 |
| G033127 | − | GTCCCCATGGGCCCTACTGG | GGG | 560 |
| G033128 | − | AATCCCCCAGGTGGTAGCCC | AGG | 561 |
| G033129 | + | CATGAGCCTACTCCTTCAGT | GGG | 562 |
| G033130 | − | TCCTAGTTCCAGTTCAAGAC | AGG | 563 |
| G033131 | − | GGTCCCCATGGGCCCTACTG | GGG | 564 |
| G033132 | + | CCACCACGCATTAGTCCCTA | TGG | 565 |
| G033133 | − | TGGGTGGACCCCATTCCACA | GGG | 566 |
| G033134 | − | AGTTAGACATGCCAATATGC | CGG | 567 |
| G033135 | − | AGTGCAAAGATTAGCTTGTC | AGG | 568 |
| G033136 | − | TGGGTCCCCATGGGCCCTAC | TGG | 569 |
| G033137 | + | ATGCCCTTGTCATTCAGGTT | GGG | 570 |
| G033138 | + | TAGTCCCTATGGCTGCATCA | TGG | 571 |
| G033139 | + | GCCTGTCTTGAACTGGAACT | AGG | 572 |
| G033140 | − | CAACCATCTGCTGTCATTGC | TGG | 573 |
| G033141 | − | ACAGTGAGATGGATACACCC | TGG | 574 |
| G033142 | − | TTTGAGAGGGATGACTCCTA | TGG | 575 |
| G033143 | + | GCCAAAAGTGGCTTGTTTGC | CGG | 576 |
| G033144 | − | GATTGTAGCGTGGTGATGGC | TGG | 577 |
| G033145 | − | CCACTGAAGGAGTAGGCTCA | TGG | 578 |
| G033146 | − | GGACAGGTTGCAGGACACGC | AGG | 579 |
| G033147 | − | GCCTGAAGCCATCCTACCCC | TGG | 580 |
| G033148 | + | TCACTGTAGGTAGCACAGCG | GGG | 581 |
| G033149 | + | CCAAGGCATGGCCTTTGACT | AGG | 582 |
| G033150 | − | ACCTGAATGACAAGGGCATT | GGG | 583 |
| G033151 | + | CCATGAGCCTACTCCTTCAG | TGG | 584 |
| G033152 | + | AAACATGGCAGTGTCTAGAC | AGG | 585 |
| G033153 | − | GGGATGACTCCTATGGGCCC | TGG | 586 |
| G033154 | − | GCTTCCATGGGTTGGAGCAT | TGG | 587 |
| G033155 | + | CTTACCCCCAGTAGGGCCCA | TGG | 588 |
| G033156 | + | GTGAACTCAGGGCTCCCCCC | AGG | 589 |
| G033157 | + | GTCCCTATGGCTGCATCATG | GGG | 590 |
| G033158 | + | GGCATGGCCTTTGACTAGGG | AGG | 591 |
| G033159 | − | GCACCCCATGATGCAGCCAT | AGG | 592 |
| G033160 | − | ATGAGTGACGGTGAGCAGAC | AGG | 593 |
| G033161 | + | TGGAAGAATCTCACCTCTCC | AGG | 594 |
| G033162 | − | CTGGGTGGACCCCATTCCAC | AGG | 595 |
| G033163 | − | TGACAGCATACTCTGGAGAT | GGG | 596 |
| G033164 | − | CAGCTCTCCTCCCTAGTCAA | AGG | 597 |
| G033165 | − | CCAGAACCAGGTGATCCCCC | AGG | 598 |
| G033166 | + | AGCCAGGTGATGGAGGCGCG | GGG | 599 |
| G033167 | − | CCTTTCCTTTTTGCGTAAGT | TGG | 600 |
| G033168 | − | TCTGGAAGGCATCTCAAGGA | TGG | 601 |
| G033169 | + | AACATGGCAGTGTCTAGACA | GGG | 602 |
| G033170 | + | TGAACTCAGGGCTCCCCCCA | GGG | 603 |
| G033171 | + | AGACCCCTGGGCTACCACCT | GGG | 604 |
| G033172 | + | TCAGCCAATGCTCCAACCCA | TGG | 605 |

TABLE 12
Mouse Assl Intron 1 Guide RNA Target Sequences.

| Mouse Ass1 gRNA | Strand | Guide RNA Target Sequence | PAM | SEQ ID NO: |
|---|---|---|---|---|
| mAss1_in1_g1/G033631 | + | AGACATCACTAATCGAGCCG | TGG | 213 |
| mAss1_in1_g2/G033632 | + | ACCGGTAGGGACCCGGCAAC | GGG | 214 |
| mAss1_in1_g3/G033633 | − | TGTGGACTTGGGCCCGTTGC | CGG | 215 |
| mAss1_in1_g4/G033634 | − | CCTATGCAGCCGCTCGTACT | TGG | 216 |
| mAss1_in1_g5/G033635 | + | GAATCTGTAGTCGTGATGTC | AGG | 217 |
| mAss1_in1_g6/G033636 | − | CAAAACTGCATCTGGCGTCC | AGG | 218 |

### (5) Lipid Nanoparticles Comprising Nuclease Agents

Lipid nanoparticles comprising the nuclease agents (e.g., CRISPR/Cas systems) are also provided. The lipid nanoparticles can alternatively or additionally comprise an ASS1 nucleic acid construct as disclosed herein. For example, the lipid nanoparticles can comprise a nuclease agent (e.g., CRISPR/Cas system), can comprise an ASS1 nucleic acid construct, or can comprise both a nuclease agent (e.g., a CRISPR/Cas system) and an ASS1 nucleic acid construct. Regarding CRISPR/Cas systems, the lipid nanoparticles can comprise the Cas protein in any form (e.g., protein, DNA, or mRNA) and/or can comprise the guide RNA(s) in any form (e.g., DNA or RNA). In one example, the lipid nanoparticles comprise the Cas protein in the form of mRNA (e.g., a modified RNA as described herein) and the guide RNA(s) in the form of RNA (e.g., a modified guide RNA as disclosed herein). As another example, the lipid nanoparticles can comprise the Cas protein in the form of protein and the guide RNA(s) in the form of RNA). In a specific example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end and/or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. As another example, Cas mRNA modifications can include substitution with N1-methyl-pseudouridine (e.g., fully substituted with N1-methyl-pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include an ASS1 nucleic acid construct as described elsewhere herein. In certain LNPs, the cargo can include an mRNA encoding a Cas nuclease, such as Cas9, a guide RNA or a nucleic acid encoding a guide RNA, and an ASS1 nucleic acid construct. In some LNPs, the lipid component comprises an amine lipid such as a biodegradable, ionizable lipid. In some instances, the lipid component comprises biodegradable, ionizable lipid, cholesterol, DSPC, and PEG-DMG. For example, Cas9 mRNA and gRNA can be delivered to cells and animals utilizing lipid formulations comprising ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z, 12Z)-octadeca-9,12-dienoate), cholesterol, DSPC, and PEG2k-DMG.

In some examples, the LNPs comprise cationic lipids. In some examples, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., WO 2019/067992, WO 2017/173054, WO 2015/095340, and WO 2014/136086, each of which is herein incorporated by reference in its entirety for all purposes. In some examples, the LNPs comprise molar ratios of a cationic lipid amine to RNA phosphate (N:P) of about 4.5, about 5.0, about 5.5, about 6.0, or about 6.5. In some examples, the terms cationic and ionizable in the context of LNP lipids are interchangeable (e.g., wherein ionizable lipids are cationic depending on the pH).

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy)hexadecanoyl)oxy)propane-1,3-diyl(9Z,9′Z,12Z,12′Z)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as [(6Z,9Z,28Z,31Z)-heptatriaconta-6,9,28,31-tetraen-19-yl] 4-(dimethylamino)butanoate or Dlin-MC3-DMA (MC3))).

Some such lipids suitable for use in the LNPs described herein are biodegradable in vivo. For example, LNPs comprising such a lipid include those where at least 75% of the lipid is cleared from the plasma within 8, 10, 12, 24, or 48 hours, or 3, 4, 5, 6, 7, or 10 days. As another example, at least 50% of the LNP is cleared from the plasma within 8, 10, 12, 24, or 48 hours, or 3, 4, 5, 6, 7, or 10 days.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine or 1,2-distearoyl-sn-glycero-3-phosphocholine (DSPC), phosphocholine (DOPC), dimyristoylphosphatidylcholine (DMPC), phosphatidylcholine (PLPC), 1,2-diarachidonoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, 1-stearoyl-2-oleoyl-sn-glycero-3-phosphocholine (SOPC), and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the LNP. Suitable stealth lipids include lipids having a hydrophilic head group linked to a lipid moiety.

The hydrophilic head group of a stealth lipid can comprise, for example, a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids, and poly N-(2-hydroxypropyl)methacrylamide. The term PEG means any polyethylene glycol or other polyalkylene ether polymer. In certain LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000](PEG2k-DMPE), or 1,2-dimyristoyl-rac-glycero-3-methylpolyoxyethylene glycol-2000 (PEG2k-DMG), 1,2-distearoyl-sn-glycero-3-phosphoethanolamine-N-[methoxy(polyethylene glycol)-2000](PEG2k-DSPE), 1,2-distearoyl-sn-glycerol, methoxypoly ethylene glycol (PEG2k-DSG), poly(ethylene glycol)-2000-dimethacrylate (PEG2k-DMA), and 1,2-distearyloxypropyl-3-amine-N-[methoxy(polyethylene glycol)-2000] (PEG2k-DSA). In one particular example, the stealth lipid may be PEG2k-DMG.

In some embodiments, the PEG lipid includes a glycerol group. In some embodiments, the PEG lipid includes a dimyristoylglycerol (DMG) group. In some embodiments, the PEG lipid comprises PEG2k. In some embodiments, the PEG lipid is a PEG-DMG. In some embodiments, the PEG lipid is a PEG2k-DMG. In some embodiments, the PEG lipid is 1,2-dimyristoyl-rac-glycero-3-methoxypolyethylene glycol-2000. In some embodiments, the PEG2k-DMG is 1,2-dimyristoyl-rac-glycero-3-methoxypolyethylene glycol-2000.

The LNPs can comprise different respective molar ratios of the component lipids in the formulation. The mol-% of the CCD lipid may be, for example, from about 30 mol-% to about 60 mol-%, from about 35 mol-% to about 55 mol-%, from about 40 mol-% to about 50 mol-%, from about 42 mol-% to about 47 mol-%, or about 45%. The mol-% of the helper lipid may be, for example, from about 30 mol-% to about 60 mol-%, from about 35 mol-% to about 55 mol-%, from about 40 mol-% to about 50 mol-%, from about 41 mol-% to about 46 mol-%, or about 44 mol-%. The mol-% of the neutral lipid may be, for example, from about 1 mol-% to about 20 mol-%, from about 5 mol-% to about 15 mol-%, from about 7 mol-% to about 12 mol-%, or about 9 mol-%. The mol-% of the stealth lipid may be, for example, from about 1 mol-% to about 10 mol-%, from about 1 mol-% to about 5 mol-%, from about 1 mol-% to about 3 mol-%, about 2 mol-%, or about 1 mol-%.

The LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100, from about 1 to about 50, from about 1 to about 25, from about 1 to about 10, from about 1 to about 7, from about 3 to about 5, from about 4 to about 5, about 4, about 4.5, or about 5. The N/P ratio can also be from about 4 to about 7 or from about 4.5 to about 6. In specific examples, the N/P ratio can be 4.5 or can be 6.

In some LNPs, the cargo can comprise Cas mRNA (e.g., Cas9 mRNA) and gRNA. The Cas mRNA and gRNAs can be in different ratios. For example, the LNP formulation can include a ratio of Cas mRNA to gRNA nucleic acid ranging from about 25:1 to about 1:25, ranging from about 10:1 to about 1:10, ranging from about 5:1 to about 1:5, or about 1:1. Alternatively, the LNP formulation can include a ratio of Cas mRNA to gRNA nucleic acid from about 1:1 to about 1:5, or about 10:1. Alternatively, the LNP formulation can include a ratio of Cas mRNA to gRNA nucleic acid of about 1:10, 25:1, 10:1, 5:1, 3:1, 1:1, 1:3, 1:5, 1:10, or 1:25. Alternatively, the LNP formulation can include a ratio of Cas mRNA to gRNA nucleic acid of from about 2:1 to about 1:2. Alternatively, the LNP formulation can include a ratio of Cas mRNA to gRNA nucleic acid of from about 1:1 to about 1:2. In specific examples, the ratio of Cas mRNA to gRNA can be about 1:1. In specific examples, the ratio of Cas mRNA to gRNA can be about 1:2. In specific examples, the ratio of Cas mRNA to gRNA can be about 2:1.

Exemplary dosing of LNPs includes about 0.1, about 0.25, about 0.3, about 0.5, about 1, about 2, about 3, about 4, about 5, about 6, about 8, or about 10 mg/kg body weight (mpk) or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 1, about 3, or about 10 mg/kg can be used. Additional exemplary dosing of LNPs includes about 0.1, about 0.25, about 0.3, about 0.5, about 1, about 2, about 3, about 4, about 5, about 6, about 8, or about 10 mg/kg (mpk) body weight or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 0.5, about 1, about 2, about 3, or about 10 mg/kg can be used. In another example, LNP doses between about 0.5 and about 10, between about 0.5 and about 5, between about 0.5 and about 3, between about 1 and about 10, between about 1 and about 5, between about 1 and about 3, or between about 1 and about 2 mg/kg can be used. In another example, LNP doses between about 0.5 and about 3, between about 0.5 and about 2.5, between about 0.5 and about 2, between about 0.5 and about 1.5, between about 0.5 and about 1, between about 1 and about 3, between about 1 and about 2.5, between about 1 and about 2, or between about 1 and about 1.5 mg/kg can be used. In another example, an LNP dose of about 1 mg/kg can be used.

In some LNPs, the cargo can comprise an ASS1 nucleic acid construct and gRNA. The ASS1 nucleic acid construct and gRNAs can be in different ratios. For example, the LNP formulation can include a ratio of ASS1 nucleic acid construct to gRNA nucleic acid ranging from about 25:1 to about 1:25, ranging from about 10:1 to about 1:10, ranging from about 5:1 to about 1:5, or about 1:1. Alternatively, the LNP formulation can include a ratio of ASS1 nucleic acid construct to gRNA nucleic acid from about 1:1 to about 1:5, about 5:1 to about 1:1, about 10:1, or about 1:10. Alternatively, the LNP formulation can include a ratio of ASS1 nucleic acid construct to gRNA nucleic acid of about 1:10, about 25:1, about 10:1, about 5:1, about 3:1, about 1:1, about 1:3, about 1:5, about 1:10, or about 1:25.

A specific example of a suitable LNP has a nitrogen-to-phosphate (N/P) ratio of about 4.5 and contains biodegradable cationic lipid, cholesterol, DSPC, and PEG2k-DMG in an about 45:44:9:2 molar ratio (about 45:about 44:about 9:about 2). The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235, herein incorporated by reference in its entirety for all purposes. The Cas9 mRNA can be in an about 1:1 (about 1:about 1) ratio by weight to the guide RNA. Another specific example of a suitable LNP contains Dlin-MC3-DMA (MC3), cholesterol, DSPC, and PEG-DMG in an about 50:38.5:10:1.5 molar ratio (about 50:about 38.5:about 10:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP has a nitrogen-to-phosphate (N/P) ratio of about 6 and contains biodegradable cationic lipid, cholesterol, DSPC, and PEG2k-DMG in an about 50:38:9:3 molar ratio (about 50:about 38:about 9:about 3). The biodegradable cationic lipid can be Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 (about 2:about 1) ratio by weight to the guide RNA.

Another specific example of a suitable LNP has a nitrogen-to-phosphate (N/P) ratio of about 3 and contains a cationic lipid, a structural lipid, cholesterol (e.g., cholesterol (ovine) (Avanti 700000)), and PEG2k-DMG (e.g., PEG-DMG 2000 (NOF America-SUNBRIGHT® GM-020 (DMG-PEG)) in an about 50:10:38.5:1.5 ratio (about 50:about 10:about 38.5:about 1.5) or an about 47:10:42:1 ratio (about 47:about 10:about 42:about 1). The structural lipid can be, for example, DSPC (e.g., DSPC (Avanti 850365)), SOPC, DOPC, or DOPE. The cationic/ionizable lipid can be, for example, Dlin-MC3-DMA (e.g., Dlin-MC3-DMA (Biofine International)). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP contains Dlin-MC3-DMA, DSPC, cholesterol, and a PEG lipid in an about 45:9:44:2 ratio (about 45:about 9:about 44:about 2). Another specific example of a suitable LNP contains Dlin-MC3-DMA, DOPE, cholesterol, and PEG lipid or PEG DMG in an about 50:10:39:1 ratio (about 50:about 10:about 39:about 1). Another specific example of a suitable LNP has Dlin-MC3-DMA, DSPC, cholesterol, and PEG2k-DMG at an about 55:10:32.5:2.5 ratio (about 55:about 10:about 32.5:about 2.5). Another specific example of a suitable LNP has Dlin-MC3-DMA, DSPC, cholesterol, and PEG-DMG in an about 50:10:38.5:1.5 ratio (about 50:about 10:about 38.5:about 1.5). Another specific example of a suitable LNP has Dlin-MC3-DMA, DSPC, cholesterol, and PEG-DMG in an about 50:10:38.5:1.5 ratio (about 50:about 10:about 38.5:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Other examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes.

### (6) Vectors Comprising Nuclease Agents

The nuclease agents disclosed herein (e.g., ZFN, TALEN, or CRISPR/Cas) can be provided in a vector for expression. A vector can comprise additional sequences such as, for example, replication origins, promoters, and genes encoding antibiotic resistance.

Some vectors may be circular. Alternatively, the vector may be linear. The vector can be in the packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

Introduction of nucleic acids can also be accomplished by virus-mediated delivery, such as AAV-mediated delivery or lentivirus-mediated delivery. The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression (e.g., of Cas and/or gRNA). Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹², about 10¹³, about 10¹⁴, about 10¹⁵, and about 10¹⁶ vector genomes (vg)/mL, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/mL. Other exemplary viral titers (e.g., AAV titers) include about 10¹², about 10¹³, about 10¹⁴, about 10¹⁵, and about 10¹⁶ vector genomes (vg)/kg of body weight, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/kg of body weight. In one example, the viral titer is between about 10¹³ to about 10¹⁴ vg/mL or vg/kg. In another example, the viral titer is between about 10¹² to about 10¹³ vg/mL or vg/kg (e.g., between about 10¹² to about 10¹³ vg/kg). In another example, the viral titer is between about 10¹² to about 10¹⁴ vg/mL or vg/kg (e.g., between about 10¹² to about 10¹⁴ vg/kg). For example, the viral titer can be between about 1.5E12 to about 1.5E13 vg/kg, can be about 1.5E12 vg/kg, or can be about 1.5E13 vg/kg. In another example, the viral titer is about 2E13 vg/mL or vg/kg.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

Some non-limiting examples of ITRs that can be used include ITRs comprising, consisting essentially of, or consisting of SEQ ID NO: 241, SEQ ID NO: 242, or SEQ ID NO: 243. Other examples of ITRs comprise one or more mutations compared to SEQ ID NO: 241, SEQ ID NO: 242, or SEQ ID NO: 243 and can be at least 90%, at least 91%, at least 92%, at least 93%, at least 94%, at least 95%, at least 96%, at least 97%, at least 98%, or at least 99% identical to SEQ ID NO: 241, SEQ ID NO: 242, or SEQ ID NO: 243. In some rAAV genomes disclosed herein, the nucleic acid encoding the nuclease agent (or component thereof) is flanked on both sides by the same ITR (i.e., the ITR on the 5′ end, and the reverse complement of the ITR on the 3′ end). In one example, the ITR on each end can comprise, consist essentially of, or consist of SEQ ID NO: 241. In another example, the ITR on each end can comprise, consist essentially of, or consist of SEQ ID NO: 242. In one example, the ITR on at least one end comprises, consists essentially of, or consists of SEQ ID NO: 243. In one example, the ITR on the 5′ end comprises, consists essentially of, or consists of SEQ ID NO: 243. In one example, the ITR on the 3′ end comprises, consists essentially of, or consists of SEQ ID NO: 243. In one example, the ITR on each end can comprise, consist essentially of, or consist of SEQ ID NO: 243. In one example, the ITR on at least one end comprises, consists essentially of, or consists of SEQ ID NO: 241. In one example, the ITR on the 5′ end comprises, consists essentially of, or consists of SEQ ID NO: 241. In one example, the ITR on the 3′ end comprises, consists essentially of, or consists of SEQ ID NO: 241. In one example, the ITR on each end can comprise, consist essentially of, or consist of SEQ ID NO: 241. In other rAAV genomes disclosed herein, the nucleic acid encoding the nuclease agent (or component thereof) is flanked by different ITRs on each end. In one example, the ITR on one end comprises, consists essentially of, or consists of SEQ ID NO: 241, and the ITR on the other end comprises, consists essentially of, or consists of SEQ ID NO: 242. In another example, the ITR on one end comprises, consists essentially of, or consists of SEQ ID NO: 241, and the ITR on the other end comprises, consists essentially of, or consists of SEQ ID NO: 243. In one example, the ITR on one end comprises, consists essentially of, or consists of SEQ ID NO: 242, and the ITR on the other end comprises, consists essentially of, or consists of SEQ ID NO: 243.

The specific serotype of a recombinant AAV vector influences its in-vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

When specific rAAVs comprising specific sequences (e.g., specific bidirectional construct sequences or specific unidirectional construct sequences) are disclosed herein, they are meant to encompass the sequence disclosed or the reverse complement of the sequence. For example, if a bidirectional or unidirectional construct disclosed herein consists of the hypothetical sequence 5′-CTGGACCGA-3′, it is also meant to encompass the reverse complement of that sequence (5′-TCGGTCCAG-3′). Likewise, when rAAVs comprising bidirectional or unidirectional construct elements in a specific 5′ to 3′ order are disclosed herein, they are also meant to encompass the reverse complement of the order of those elements. For example, if an rAAV is disclosed herein that comprises a bidirectional construct that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., Ling et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding a heterologous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector. In another specific example, the AAV vector comprising the nucleic acid construct can be recombinant AAV5 (rAAV5). A rAAV5 vector as described herein is one in which the capsid is from AAV5. For example, an AAV vector using ITRs from AAV2 and a capsid of AAV5 is considered herein to be a rAAV5 vector.

Tropism can be further refined through pseudotyping, which is the mixing of a capsid and a genome from different viral serotypes. For example, AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

The vector (e.g., AAV such as recombinant AAV8 or recombinant AAV5) can be formulated, for example, in 10 mM sodium phosphate, 180 mM sodium chloride, and 0.005% poloxamer 188, at pH 7.3.

In certain AAVs, the cargo can include nucleic acids encoding one or more guide RNAs (e.g., DNA encoding a guide RNA, or DNA encoding two or more guide RNAs). In certain AAVs, the cargo can include a nucleic acid (e.g., DNA) encoding a Cas nuclease, such as Cas9, and DNA encoding one or more guide RNAs (e.g., DNA encoding a guide RNA, or DNA encoding two or more guide RNAs). In certain AAVs, the cargo can include an ASS1 nucleic acid construct. In certain AAVs, the cargo can include a nucleic acid (e.g., DNA) encoding a Cas nuclease, such as Cas9, a DNA encoding a guide RNA (or multiple guide RNAs), and an ASS1 nucleic acid construct.

For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., rAAV8-mediated delivery or rAAV5-mediated delivery). For example, a Cas9 mRNA and a gRNA can be delivered via LNP-mediated delivery, or DNA encoding Cas9 and DNA encoding a gRNA can be delivered via AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

### C. Cells or Animals or Genomes

Cells or animals (i.e., subjects) comprising any of the above compositions (e.g., ASS1 nucleic acid constructs, nuclease agents, vectors, lipid nanoparticles, or any combination thereof) are also provided herein. Such cells or animals (or genomes) can be produced by the methods disclosed herein. For example, the cells or animals can comprise any of the ASS1 nucleic acid constructs described herein, any of the nuclease agents disclosed herein, or both. Such cells or animals (or genomes) can be neonatal cells or animals (or genomes). Alternatively, such cells or animals (or genomes) can be non-neonatal cells or animals (or genomes).

A neonatal subject (e.g., animal) can be a human subject up to or under the age of 1 year (52 weeks), preferably up to or under the age of 24 weeks, more preferably up to or under the age of 12 weeks, more preferably up to or under the age of 8 weeks, even more preferably up to or under the age of 4 weeks, even more preferably up to or under the age of 2 weeks, and even more preferably up to or under the age of 1 week. In certain embodiments, a neonatal human subject is up to 1 week of age. In certain embodiments, a neonatal human subject is up to 2 weeks of age. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject.

Neonatal cells can be of any neonatal subject. For example, they can be from a human subject up to or under the age of 1 year (52 weeks), preferably up to or under the age of 24 weeks, more preferably up to or under the age of 12 weeks, more preferably up to or under the age of 8 weeks, even more preferably up to or under the age of 4 weeks, even more preferably up to or under the age of 2 weeks, and even more preferably up to or under the age of 1 week. In certain embodiments, a neonatal human subject is up to 1 week of age. In certain embodiments, a neonatal human subject is up to 2 weeks of age. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject.

In some such cells, animals, or genomes, the ASS1 nucleic acid construct can be genomically integrated at a target genomic locus, such as an endogenous ASS1 locus (e.g., a human ASS1 locus, such as intron 2 of a human ASS1 locus, intron 1 of a human ASS1 locus, or intron 1 of a mouse Ass1 locus). In some such cells, animals, or genomes, the ASS1 nucleic acid construct is expressed in the cell, animal, or genome. For example, if the ASS1 nucleic acid construct is integrated into an ASS1 locus (e.g., intron 2 of a human ASS1 locus or intron 1 of a human ASS1 locus), ASS1 protein can be expressed from the ASS1 locus. The ASS1 coding sequence can be operably linked to an endogenous promoter at the target genomic locus upon integration into the target genomic locus, or it can be operably linked to an exogenous promoter present in the nucleic acid construct. If the nucleic acid construct is a bidirectional nucleic acid construct disclosed herein, the genome, cell, or animal can express the first ASS1 protein or can express the second ASS1 protein. In some genomes, cells, or animals, the target genomic locus is an ASS1 locus. For example, the nucleic acid construct can be genomically integrated in intron 2 of the endogenous human ASS1 locus. Endogenous human ASS1 exons 1 and 2 can then splice into the coding sequence for the ASS1 protein in the nucleic acid construct. As another example, the nucleic acid construct can be genomically integrated in intron 1 of the endogenous human ASS1 locus. Endogenous human ASS1 exon 1 can then splice into the coding sequence for the ASS1 protein in the nucleic acid construct.

The target genomic locus at which the nucleic acid construct is stably integrated can be heterozygous for the ASS1 coding sequence from the nucleic acid construct or homozygous for the ASS1 coding sequence from the nucleic acid construct. A diploid organism has two alleles at each genetic locus. Each pair of alleles represents the genotype of a specific genetic locus. Genotypes are described as homozygous if there are two identical alleles at a particular locus and as heterozygous if the two alleles differ.

The cells, animals, or genomes can be from any suitable species, such as eukaryotic cells or eukaryotes, or mammalian cells or mammals (e.g., non-human mammalian cells or non-human mammals, or human cells or humans). A mammal can be, for example, a non-human mammal, a human, a rodent, a rat, a mouse, or a hamster. Other non-human mammals include, for example, non-human primates, monkeys, apes, cats, dogs, rabbits, horses, bulls, deer, bison, livestock (e.g., bovine species such as cows, steer, and so forth; ovine species such as sheep, goats, and so forth; and porcine species such as pigs and boars). Birds include, for example, chickens, turkeys, ostrich, geese, ducks, and so forth. Domesticated animals and agricultural animals are also included. The term “non-human” excludes humans. Examples include, but are not limited to, human cells/humans, rodent cells/rodents, mouse cells/mice, rat cells/rats, and non-human primate cells/non-human primates. In a specific example, the cell is a human cell or the animal is a human. Likewise, cells can be any suitable type of cell. In a specific example, the cell is a liver cell such as a hepatocyte (e.g., a human liver cell or human hepatocyte).

The cells can be isolated cells (e.g., in vitro), ex vivo cells, or can be in vivo within an animal (i.e., in a subject). The cells can be mitotically competent cells or mitotically inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the cells can be liver cells, such as hepatocytes (e.g., mouse, non-human primate, or human hepatocytes).

The cells provided herein can be normal, healthy cells, or can be diseased or mutant-bearing cells. For example, the cells can have an ASS1 deficiency or can be from a subject with ASS1 deficiency or citrullinemia type I.

The cells provided herein can be non-dividing cells. A non-dividing cell refers to cells that are terminally differentiated and do not divide, as well as quiescent cells that do not divide but retains the ability to re-enter cell division and proliferation. Liver cells, for example, retain the ability to divide (e.g., when injured or resected), but do not typically divide. During mitotic cell division, homologous recombination is a mechanism by which the genome is protected and double-stranded breaks are repaired. A non-dividing cell can refer to a cell in which homologous recombination (HR) is not the primary mechanism by which double-stranded DNA breaks are repaired in the cell (e.g., as compared to a control dividing cell). A non-dividing cell can refer to a cell in which non-homologous end joining (NHEJ) is the primary mechanism by which double-stranded DNA breaks are repaired in the cell (e.g., as compared to a control dividing cell). Non-dividing cell types have been described in the literature, for example, by active NHEJ double-stranded DNA break repair mechanisms. See, e.g., Iyama, DNA Repair (Amst.) 2013, 12(8): 620-636.

### III. Methods for Introducing, Integrating, or Expressing an ASS1 Nucleic Acid or for Treatment of Citrullinemia Type I

The nucleic acid constructs and compositions disclosed herein can be used in methods of introducing an ASS1 nucleic acid into a cell, methods of integration of an ASS1 nucleic acid into a target genomic locus, methods of expression of ASS1 in a cell, and in methods of treating citrullinemia type I or ASS1 deficiency in a subject. The nucleic acid constructs and compositions disclosed herein can be used in methods of introducing an ASS1 nucleic acid into a cell or a population of cells or a subject, methods of inserting or integrating an ASS1 nucleic acid into a target genomic locus, methods of expressing an ASS1 protein in a cell or a population of cells or a subject, and in methods of treating citrullinemia type I or ASS1 deficiency in a subject.

The nucleic acid constructs and compositions disclosed herein can be used in the preparation of reagents for introducing an ASS1 nucleic acid into a cell, integrating an ASS1 nucleic acid into a target genomic locus, or expressing ASS1 in a cell. The nucleic acid constructs and compositions disclosed herein can be used in preparation of medicaments for treating citrullinemia type I or ASS1 deficiency in a subject. The nucleic acid constructs and compositions disclosed herein can be used in the preparation of reagents for introducing an ASS1 nucleic acid into a cell or a population of cells or a subject, inserting or integrating an ASS1 nucleic acid into a target genomic locus, or expressing an ASS1 protein in a cell or a population of cells or a subject.

### A. Citrullinemia Type I

The compositions disclosed herein (e.g., ASS1 nucleic acid constructs, or ASS1 nucleic acid constructs in combination with the nuclease agents (e.g., CRISPR/Cas systems) are useful for the treatment of ASS1 deficiency or citrullinemia type I and/or ameliorating at least one symptom associated with ASS1 deficiency or citrullinemia type I. Likewise, the compositions disclosed herein can be used for the preparation of a pharmaceutical composition or medicament for treating a subject having ASS1 deficiency or citrullinemia type I.

Citrullinemia type I (CTLN1) is an autosomal recessive urea cycle disorder. The urea cycle is the main ammonia detoxification pathway in humans, active in periportal hepatocytes. CTLN1 is the second most common urea cycle disorder. In CTLN1, there is a deficiency in the enzyme argininosuccinate synthase (ASS1), which catalyzes the condensation of citrulline and aspartate to argininosuccinate. This deficiency in ASS1 leads to an accumulation of upstream metabolites citrulline and ammonia. If left untreated, this accumulation of upstream metabolites results in toxic levels of ammonia (hyperammonemia).

Clinical Characteristics. CTLN1 presents as a spectrum that includes a neonatal acute form (the “classic” form), a milder late-onset form in which women have onset of symptoms at pregnancy or postpartum (the “non-classic” form), as well a form without symptoms or hyperammonemia. Distinction between the forms is based primarily on clinical findings, although emerging evidence suggests that measurement of residual argininosuccinate synthase enzyme activity may help to predict those who are likely to have a severe phenotype and those who are likely to have an attenuated phenotype.

Infants with the acute neonatal form appear normal at birth. Shortly thereafter, they develop hyperammonemia and become progressively lethargic, feed poorly, often vomit, and may develop signs of increased intracranial pressure (ICP). Without prompt intervention, hyperammonemia and the accumulation of other toxic metabolites (e.g., glutamine) result in increased ICP, increased neuromuscular tone, spasticity, ankle clonus, seizures, loss of consciousness, and death. Children with the severe form who are treated promptly may survive for an indeterminate period of time, but usually with significant neurologic deficits. Even with chronic protein restriction and scavenger therapy, long-term complications such as liver failure and other (rarely reported) organ system manifestations are possible.

The late-onset form may be milder than that seen in the acute neonatal form, but commences later in life for reasons that are not completely understood. The episodes of hyperammonemia are similar to those seen in the acute neonatal form, but the initial neurologic findings may be more subtle because of the older age of the affected individuals. Women with onset of severe symptoms including acute hepatic decompensation during pregnancy or in the postpartum period have been reported. Furthermore, previously asymptomatic and non-pregnant individuals have been described who remained asymptomatic up to at least age ten years, with the possibility that they could remain asymptomatic lifelong.

Diagnosis/Testing. The diagnosis of CTLN1 is established in a proband with elevated plasma ammonia concentration (>150 μmol/L; may range to ≥2000-3000 μmol/L), elevated plasma citrulline concentration (usually >500 μmol/L), and absent argininosuccinate and/or by identification of biallelic pathogenic variants in ASS1 on molecular genetic testing. Measurement of ASS1 enzyme activity is not currently widely used because the clinical presentation and relatively specific pattern of metabolites found in affected individuals are sufficient to establish the diagnosis. Historically, determining the prognosis prospectively was difficult in some individuals who fit the biochemical phenotype but may or may not have had serious clinical illness. Newer data suggest that individuals with ≥8% residual ASS1 enzymatic activity have less frequent and less severe hyperammonemic events, and better cognition. A cutoff value of 8% residual enzymatic activity has been proposed as a threshold for discrimination between severe (≤8% activity) and mild-to-moderate (>8% activity) disease (see “ASS Enzyme Activity” in Zielonka et al. (2019) Ann. Clin. Transl. Neurol. 6:1858-71).

Management. Treatment of manifestations: Liver transplantation is the only known curative therapy and eliminates the need for dietary restriction. Transplantation is ideally performed in affected individuals who are younger than age one year (prior to the development of any neurocognitive impairment) but older than age three months and/or above 5 kg body weight.

Daily routine treatment in those who have not undergone a liver transplantation includes lifelong protein restriction in conjunction with a metabolic nutritionist; nitrogen scavenger medications; arginine supplementation; consideration of carnitine supplementation in those with secondary carnitine deficiency; addressing increased energy/caloric demands through tube feedings (as needed); and routine treatment of developmental delay/intellectual disability.

Acute inpatient treatment of a metabolic crisis includes addressing hyperammonemia through withholding of all protein intake for a maximum of 24 to 28 hours; pharmacologic nitrogen scavenger therapy; and consideration of dialysis (the most effective means of reducing plasma ammonia concentration rapidly). To address increased catabolism, administration of high-energy fluids (and insulin, as needed) and intravenous intralipids is typically required. However, care must be taken to avoid electrolyte imbalance and fluid overload, which can contribute to the development of increased intracranial pressure. The patient should be maintained on the dry side of fluid balance (approximately 85 mL/kg of body weight per day in infants and appropriate corresponding fluid restriction in children and adults).

Prevention of secondary complications: Education of parents and caregivers such that diligent observation and management can be administered expediently in the setting of intercurrent illness or other catabolic stressors; written protocols for maintenance and emergency treatment should be provided to parents and primary care providers/pediatricians, and to teachers and school staff. For those affected individuals requiring any sedated procedure where a person cannot eat for an extended period of time, drug treatment should be switched to IV and nutrition with 10% glucose with age-appropriate electrolytes should be administered via IV to promote anabolism starting as soon as the patient is NPO.

Surveillance: Follow up in a metabolic clinic with a qualified metabolic nutritionist and clinical biochemical geneticist is required. Measurement of growth parameters; evaluation of nutrition status and safety of oral intake; assessment for early warning signs of impending hyperammonemic episodes (mood changes, headache, lethargy, nausea, refusal to eat); review of dietary assessment; monitoring of developmental progress/educational needs; assessment of mobility and self-help skills; and measurement of carnitine levels (for those on sodium benzoate) at each visit. Plasma amino acid analysis at least every three months during the first year of life and every six to 12 months in the teenage/adult years (depending on clinical stability).

Agents/circumstances to avoid: Excessive protein intake, prolonged fasting, and obvious exposure to communicable diseases.

Evaluation of relatives at risk: It is important that at-risk sibs be identified as soon as possible, either through molecular genetic testing (if the pathogenic variants in the family are known) or measurement of plasma concentrations of ammonia and citrulline on the first day of life. Elevation of either above acceptable levels (ammonia >100 μmol/L or plasma citrulline >˜100 μmol/L) is sufficient evidence to initiate treatment in a newborn.

Pregnancy management: Because women with onset of severe symptoms during pregnancy or in the postpartum period have been reported, scrupulous attention needs to be paid to diet and medication during these periods.

Genetic Counseling. CTLN1 is inherited in an autosomal recessive manner. At conception, each sib of an affected individual has a 25% chance of being affected, a 50% chance of being an asymptomatic carrier, and a 25% chance of being unaffected and not a carrier. Carrier testing for at-risk relatives and prenatal testing for a pregnancy at increased risk are possible if the pathogenic variants in the family are known.

### B. Methods

Methods of introducing an ASS1 nucleic acid into a cell, methods of integration of an ASS1 nucleic acid into a target genomic locus, methods of expression of ASS1 in a cell, and methods of treating citrullinemia type I or ASS1 deficiency in a subject are provided. Methods of introducing an ASS1 nucleic acid into a cell or a population of cells (or in a cell or population of cells in a subject), methods of inserting an ASS1 nucleic acid into a target genomic locus in a cell or a population of cells (or in a cell or population of cells in a subject), methods of expressing an ASS1 protein in a cell or a population of cells (or in a cell or population of cells in a subject), methods of treating citrullinemia type I or ASS1 deficiency in a subject are provided.

The cells or populations of cells can be neonatal cells or populations of neonatal cells, and the subject can be neonatal subjects in some methods. A neonatal subject can be a human subject up to or under the age of 1 year (52 weeks), preferably up to or under the age of 24 weeks, more preferably up to or under the age of 12 weeks, more preferably up to or under the age of 8 weeks, even more preferably up to or under the age of 4 weeks, even more preferably up to or under the age of 2 weeks, and even more preferably up to or under the age of 1 week. In certain embodiments, a neonatal human subject is up to 1 week of age. In certain embodiments, a neonatal human subject is up to 2 weeks of age. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject.

The cells or populations of cells can be juvenile cells or populations of juvenile cells, and the subject can be juvenile subjects in some methods. A juvenile subject in the context of humans cover human subjects under the age of 18 years. In one embodiment, a human juvenile subject is a subject within 17 years after birth. In another embodiment, a human juvenile subject is a subject within 16 years after birth. In another embodiment, a human juvenile subject is a subject within 15 years after birth. In another embodiment, a human juvenile subject is a subject within 14 years after birth. In another embodiment, a human juvenile subject is a subject within 13 years after birth. In another embodiment, a human juvenile subject is a subject within 12 years after birth. In another embodiment, a human juvenile subject is a subject within 11 years after birth. In another embodiment, a human juvenile subject is a subject within 10 years after birth. In another embodiment, a human juvenile subject is a subject within 9 years after birth. In another embodiment, a human juvenile subject is a subject within 8 years after birth. In another embodiment, a human juvenile subject is a subject within 7 years after birth. In another embodiment, a human juvenile subject is a subject within 6 years after birth. In another embodiment, a human juvenile subject is a subject within 5 years after birth. In another embodiment, a human juvenile subject is a subject within 4 years after birth. In another embodiment, a human juvenile subject is a subject within 3 years after birth. In another embodiment, a human juvenile subject is a subject within 2 years after birth. In another embodiment, a human juvenile subject is a subject within 1 year after birth. The time windows disclosed above are for human subjects and are also meant to cover the corresponding developmental time windows for other animals. As used herein, a juvenile cell is a cell of a juvenile subject, and a population of juvenile cells is a population of cells of a juvenile subject.

In some methods, the subject has an actively growing liver, or the cells or populations of cells are from an actively growing liver. In other methods, the subject does not have an actively growing liver, or the cells or populations of cells are not from an actively growing liver.

In one example, provided herein are methods of introducing an ASS1 nucleic acid into a cell or a subject in need thereof. Such methods can comprise administering any of the ASS1 nucleic acid constructs described herein (or any of the compositions comprising an ASS1 nucleic acid construct described herein, including, for example, vectors or lipid nanoparticles) to the cell. The ASS1 nucleic acid construct can be administered together with a nuclease agent described herein, or can be administered alone. In some methods, the ASS1 nucleic acid construct can be administered together with a nuclease agent described herein. The nuclease agent can cleave a nuclease target sequence within a target gene to create a cleavage site, the ASS1 nucleic acid construct can be inserted into the cleavage site to create a modified target gene, and ASS1 protein can be expressed from the modified target gene. The ASS1 coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ASS1 (e.g., intron 2 of human ASS1 or intron 1 of human ASS1). In such methods, the guide RNA can bind to the Cas protein and target the Cas protein to the guide RNA target sequence in intron 2 of the ASS1 gene, the Cas protein can cleave the guide RNA target sequence to create a cleavage site, the nucleic acid construct can be inserted into the cleavage site to create a modified ASS1 gene, and ASS1 protein can be expressed from the modified ASS1 gene. In another example, the guide RNA can bind to the Cas protein and target the Cas protein to the guide RNA target sequence in intron 1 of the ASS1 gene, the Cas protein can cleave the guide RNA target sequence to create a cleavage site, the nucleic acid construct can be inserted into the cleavage site to create a modified ASS1 gene, and ASS1 protein can be expressed from the modified ASS1 gene.

In one example, provided herein are methods of introducing an ASS1 nucleic acid into a cell or a population of cells or a subject in need thereof (e.g., in a cell or a population of cells in the subject). The cells or populations of cells can be neonatal cells or populations of neonatal cells, and the subject can be neonatal subjects in some methods. In other methods, the cells or populations of cells are not neonatal cells and are not populations of neonatal cells, and the subjects are not neonatal subjects. The cells or populations of cells can be juvenile cells or populations of juvenile cells, and the subject can be juvenile subjects in some methods. In other methods, the cells or populations of cells are not juvenile cells and are not populations of juvenile cells, and the subjects are not juvenile subjects. Such methods can comprise administering any of the ASS1 nucleic acid constructs described herein (or any of the compositions comprising ASS1 nucleic acid construct described herein, including, for example, vectors or lipid nanoparticles) to the cell. The ASS1 nucleic acid construct can be administered together with a nuclease agent described herein, or can be administered alone. In some methods, the ASS1 nucleic acid construct can be administered together with a nuclease agent described herein (e.g., simultaneously or sequentially in any order). The nuclease agent can cleave a nuclease target sequence within a target genomic locus (e.g., target gene), the ASS1 nucleic acid construct can be inserted into the target genomic locus to create a modified target genomic locus, and the ASS1 protein can be expressed from the modified target genomic locus. The ASS1 coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ASS1 (e.g., intron 2 of human ASS1 or intron 1 of human ASS1). In such methods, the guide RNA can bind to the Cas protein and target the Cas protein to the guide RNA target sequence in intron 2 of the ASS1 gene, the Cas protein can cleave the guide RNA target sequence, the nucleic acid construct can be inserted into the ASS1 gene to create a modified ASS1 gene, and ASS1 protein can be expressed from the modified ASS1 gene. In another example, the guide RNA can bind to the Cas protein and target the Cas protein to the guide RNA target sequence in intron 1 of the ASS1 gene, the Cas protein can cleave the guide RNA target sequence, the nucleic acid construct can be inserted into the ASS1 gene to create a modified ASS1 gene, and ASS1 protein can be expressed from the modified ASS1 gene.

In another example, provided herein are methods of expressing ASS1 in a cell or a subject in need thereof. Such methods can comprise administering any of the ASS1 nucleic acid constructs described herein (or any of the compositions comprising an ASS1 nucleic acid construct described herein, including, for example, vectors or lipid nanoparticles) to the cell. In some methods, the ASS1 nucleic acid construct or composition comprising the ASS1 nucleic acid construct can be administered without a nuclease agent (e.g., if the ASS1 nucleic acid construct comprises elements needed for expression of ASS1 ASS1 without integration into a target genomic locus). In some methods, the ASS1 nucleic acid construct can be administered together with a nuclease agent described herein. The nuclease agent can cleave a nuclease target sequence within a target gene to create a cleavage site, the ASS1 nucleic acid construct can be inserted into the cleavage site to create a modified target gene, and ASS1 protein can be expressed from the modified target gene. The ASS1 coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ASS1 (e.g., intron 2 of human ASS1 or intron 1 of human ASS1). In such methods, the guide RNA can bind to the Cas protein and target the Cas protein to the guide RNA target sequence in intron 2 of the ASS1 gene, the Cas protein can cleave the guide RNA target sequence to create a cleavage site, the nucleic acid construct can be inserted into the cleavage site to create a modified ASS1 gene, and ASS1 protein can be expressed from the modified ASS1 gene. As another example, the guide RNA can bind to the Cas protein and target the Cas protein to the guide RNA target sequence in intron 1 of the ASS1 gene, the Cas protein can cleave the guide RNA target sequence to create a cleavage site, the nucleic acid construct can be inserted into the cleavage site to create a modified ASS1 gene, and ASS1 protein can be expressed from the modified ASS1 gene.

In another example, provided herein are methods of expressing an ASS1 protein in a cell or a population of cells or a subject in need thereof (e.g., in a cell or a population of cells in the subject). The cells or populations of cells can be neonatal cells or populations of neonatal cells, and the subject can be neonatal subjects in some methods. In other methods, the cells or populations of cells are not neonatal cells and are not populations of neonatal cells, and the subjects are not neonatal subjects. The cells or populations of cells can be juvenile cells or populations of juvenile cells, and the subject can be juvenile subjects in some methods. In other methods, the cells or populations of cells are not juvenile cells and are not populations of juvenile cells, and the subjects are not juvenile subjects. Such methods can comprise administering any of the ASS1 nucleic acid constructs described herein (or any of the compositions comprising an ASS1 nucleic acid construct described herein, including, for example, vectors or lipid nanoparticles) to the cell. In some methods, the ASS1 nucleic acid construct or composition comprising the ASS1 nucleic acid construct can be administered without a nuclease agent (e.g., if the ASS1 nucleic acid construct comprises elements needed for expression of the ASS1 protein without integration into a target genomic locus). In some methods, the ASS1 nucleic acid construct can be administered together with a nuclease agent described herein (e.g., simultaneously or sequentially in any order). The nuclease agent can cleave a nuclease target sequence within a target genomic locus (e.g., target gene), the ASS1 nucleic acid construct can be inserted into the target genomic locus to create a modified target genomic locus, and ASS1 protein can be expressed from the modified target genomic locus. The ASS1 coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ASS1 (e.g., intron 2 of human ASS1 or intron 1 of human ASS1). In such methods, the guide RNA can bind to the Cas protein and target the Cas protein to the guide RNA target sequence in intron 2 of the ASS1 gene, the Cas protein can cleave the guide RNA target sequence, the nucleic acid construct can be inserted into the ASS1 gene to create a modified ASS1 gene, and ASS1 protein can be expressed from the modified ASS1 gene. As another example, the guide RNA can bind to the Cas protein and target the Cas protein to the guide RNA target sequence in intron 1 of the ASS1 gene, the Cas protein can cleave the guide RNA target sequence, the nucleic acid construct can be inserted into the ASS1 gene to create a modified ASS1 gene, and ASS1 protein can be expressed from the modified ASS1 gene.

In another example, provided herein are methods of integrating an ASS1 nucleic acid construct into a target genomic locus in a cell or a subject in need thereof. Such methods can comprise administering any of the ASS1 nucleic acid constructs described herein (or any of the compositions comprising an ASS1 nucleic acid construct described herein, including, for example, vectors or lipid nanoparticles) to the cell. In some methods, the ASS1 nucleic acid construct or composition comprising the ASS1 nucleic acid construct can be administered together with a nuclease agent described herein. The nuclease agent can cleave a nuclease target sequence within a target gene to create a cleavage site, the ASS1 nucleic acid construct can be inserted into the cleavage site to create a modified target gene, and ASS1 protein can be expressed from the modified target gene. The ASS1 coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ASS1 (e.g., intron 2 of human ASS1 or intron 1 of human ASS1). In such methods, the guide RNA can bind to the Cas protein and target the Cas protein to the guide RNA target sequence in intron 2 of the ASS1 gene, the Cas protein can cleave the guide RNA target sequence to create a cleavage site, the nucleic acid construct can be inserted into the cleavage site to create a modified ASS1 gene, and ASS1 protein can be expressed from the modified ASS1 gene. As another example, the guide RNA can bind to the Cas protein and target the Cas protein to the guide RNA target sequence in intron 1 of the ASS1 gene, the Cas protein can cleave the guide RNA target sequence to create a cleavage site, the nucleic acid construct can be inserted into the cleavage site to create a modified ASS1 gene, and ASS1 protein can be expressed from the modified ASS1 gene.

In another example, provided herein are methods of inserting or integrating an ASS1 nucleic acid construct into a target genomic locus in a cell or a population of cells or a subject in need thereof (e.g., in a cell or a population of cells in the subject). The cells or populations of cells can be neonatal cells or populations of neonatal cells, and the subject can be neonatal subjects in some methods. In other methods, the cells or populations of cells are not neonatal cells and are not populations of neonatal cells, and the subjects are not neonatal subjects. The cells or populations of cells can be juvenile cells or populations of juvenile cells, and the subject can be juvenile subjects in some methods. In other methods, the cells or populations of cells are not juvenile cells and are not populations of juvenile cells, and the subjects are not juvenile subjects. Such methods can comprise administering any of the ASS1 nucleic acid constructs described herein (or any of the compositions comprising an ASS1 nucleic acid construct described herein, including, for example, vectors or lipid nanoparticles) to the cell. In some methods, the ASS1 nucleic acid construct or composition comprising the ASS1 nucleic acid construct can be administered together with a nuclease agent described herein (e.g., simultaneously or sequentially in any order). The nuclease agent can cleave a nuclease target sequence within a target genomic locus (e.g., target gene), the ASS1 nucleic acid construct can be inserted into the target genomic locus to create a modified target genomic locus, and the ASS1 protein can be expressed from the modified target genomic locus. The ASS1 coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ASS1 (e.g., intron 2 of human ASS1 or intron 1 of human ASS1). In such methods, the guide RNA can bind to the Cas protein and target the Cas protein to the guide RNA target sequence in intron 2 of the ASS1 gene, the Cas protein can cleave the guide RNA target sequence, the nucleic acid construct can be inserted into the ASS1 gene to create a modified ASS1 gene, and ASS1 protein can be expressed from the modified ASS1 gene. As another example, the guide RNA can bind to the Cas protein and target the Cas protein to the guide RNA target sequence in intron 1 of the ASS1 gene, the Cas protein can cleave the guide RNA target sequence, the nucleic acid construct can be inserted into the ASS1 gene to create a modified ASS1 gene, and ASS1 protein can be expressed from the modified ASS1 gene.

In any of the above methods, the cells can be from any suitable species, such as eukaryotic cells or mammalian cells (e.g., non-human mammalian cells or human cells). A mammal can be, for example, a non-human mammal, a human, a rodent, a rat, a mouse, or a hamster. Other non-human mammals include, for example, non-human primates, monkeys, apes, cats, dogs, rabbits, horses, bulls, deer, bison, livestock (e.g., bovine species such as cows, steer, and so forth; ovine species such as sheep, goats, and so forth; and porcine species such as pigs and boars). Birds include, for example, chickens, turkeys, ostrich, geese, ducks, and so forth. Domesticated animals and agricultural animals are also included. The term “non-human” excludes humans. Specific examples include, but are not limited to, human cells, rodent cells, mouse cells, rat cells, and non-human primate cells. In a specific example, the cell is a human cell. Likewise, cells can be any suitable type of cell. In a specific example, the cell is a liver cell such as a hepatocyte (e.g., a human liver cell or human hepatocyte).

The cells can be isolated cells (e.g., in vitro), ex vivo cells, or can be in vivo within an animal (i.e., in a subject). In a specific example, the cell is in vivo (e.g., in a subject having an ASS1 deficiency or citrullinemia type I). The cells can be mitotically competent cells or mitotically inactive cells, meiotically competent cells or meiotically inactive cells. Similarly, the cells can also be primary somatic cells or cells that are not a primary somatic cell. Somatic cells include any cell that is not a gamete, germ cell, gametocyte, or undifferentiated stem cell. For example, the cells can be liver cells, such as hepatocytes (e.g., mouse, non-human primate, or human hepatocytes).

The cells provided herein can be normal, healthy cells, or can be diseased or mutant-bearing cells. For example, the cells can have an ASS1 deficiency or can be from a subject with ASS1 deficiency or citrullinemia type I.

The cells can be dividing cells (e.g., actively dividing cells). The cells can also be non-dividing cells. A non-dividing cell refers to cells that are terminally differentiated and do not divide, as well as quiescent cells that do not divide but retains the ability to re-enter cell division and proliferation. Liver cells, for example, retain the ability to divide (e.g., when injured or resected), but do not typically divide. During mitotic cell division, homologous recombination is a mechanism by which the genome is protected and double-stranded breaks are repaired. A non-dividing cell can refer to a cell in which homologous recombination (HR) is not the primary mechanism by which double-stranded DNA breaks are repaired in the cell (e.g., as compared to a control dividing cell). A non-dividing cell can refer to a cell in which non-homologous end joining (NHEJ) is the primary mechanism by which double-stranded DNA breaks are repaired in the cell (e.g., as compared to a control dividing cell). Non-dividing cell types have been described in the literature, for example, by active NHEJ double-stranded DNA break repair mechanisms. See, e.g., Iyama, DNA Repair (Amst.) 2013, 12(8): 620-636

Also provided are methods of treating an ASS1 deficiency in a subject and methods of treating citrullinemia type I in a subject and methods of preventing or inhibiting hyperammonemia in a subject having citrullinemia type I. Citrullinemia type I is described in more detail elsewhere herein. Such methods can comprise administering any of the ASS1 nucleic acid constructs described herein (or any of the compositions comprising an ASS1 nucleic acid construct described herein, including, for example, vectors or lipid nanoparticles) to the subject such that a therapeutically effective level of ASS1 expression is achieved in the subject. In some methods, the ASS1 nucleic acid construct or composition comprising the ASS1 nucleic acid construct can be administered without a nuclease agent (e.g., if the ASS1 nucleic acid construct comprises elements needed for expression of ASS1 without integration into a target genomic locus). In some methods, the ASS1 nucleic acid construct can be administered together with a nuclease agent described herein. The nuclease agent can cleave a nuclease target sequence within a target gene to create a cleavage site, the ASS1 nucleic acid construct can be inserted into the cleavage site to create a modified target gene, and ASS1 protein can be expressed from the modified target gene such that a therapeutically effective level of ASS1 expression is achieved in the subject. The ASS1 coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ASS1 (e.g., intron 2 of human ASS1 or intron 1 of human ASS1). In such methods, the guide RNA can bind to the Cas protein and target the Cas protein to the guide RNA target sequence in intron 2 of the ASS1 gene, the Cas protein can cleave the guide RNA target sequence to create a cleavage site, the nucleic acid construct can be inserted into the cleavage site to create a modified ASS1 gene, and ASS1 protein can be expressed from the modified ASS1 gene such that a therapeutically effective level of ASS1 expression is achieved in the subject. As another example, the guide RNA can bind to the Cas protein and target the Cas protein to the guide RNA target sequence in intron 1 of the ASS1 gene, the Cas protein can cleave the guide RNA target sequence to create a cleavage site, the nucleic acid construct can be inserted into the cleavage site to create a modified ASS1 gene, and ASS1 protein can be expressed from the modified ASS1 gene such that a therapeutically effective level of ASS1 expression is achieved in the subject.

Also provided are methods of treating an ASS1 deficiency in a subject and methods of treating citrullinemia type I in a subject and methods of preventing or inhibiting hyperammonemia in a subject having citrullinemia type I. The subject can be a neonatal subject in some methods. In other methods, the subjects are not neonatal subjects. The subject can be a juvenile subject in some methods. In other methods, the subjects are not juvenile subjects. Citrullinemia type I is described in more detail elsewhere herein. Such methods can comprise administering any of the ASS1 nucleic acid constructs described herein (or any of the compositions comprising an ASS1 nucleic acid construct described herein, including, for example, vectors or lipid nanoparticles) to the subject such that a therapeutically effective level of ASS1 protein expression is achieved in the subject. In some methods, the ASS1 nucleic acid construct or composition comprising the ASS1 nucleic acid construct can be administered without a nuclease agent (e.g., if the ASS1 nucleic acid construct comprises elements needed for expression of ASS1 protein without integration into a target genomic locus). In some methods, the ASS1 nucleic acid construct can be administered together with a nuclease agent described herein (e.g., simultaneously or sequentially in any order). The nuclease agent can cleave a nuclease target sequence within a target genomic locus (e.g., target gene), the ASS1 nucleic acid construct can be inserted into the target genomic locus to create a modified target genomic locus, and the ASS1 protein can be expressed from the modified target genomic locus (e.g., such that a therapeutically effective level of ASS1 protein expression is achieved in the subject). The ASS1 coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ASS1 (e.g., intron 2 of ASS1 or intron 1 of ASS1). In such methods, the guide RNA can bind to the Cas protein and target the Cas protein to the guide RNA target sequence in intron 2 of the ASS1 gene, the Cas protein can cleave the guide RNA target, the nucleic acid construct can be inserted into the ASS1 gene to create a modified ASS1 gene, and ASS1 protein can be expressed from the modified ASS1 gene (e.g., such that a therapeutically effective level of ASS1 protein expression is achieved in the subject). As another example, the guide RNA can bind to the Cas protein and target the Cas protein to the guide RNA target sequence in intron 1 of the ASS1 gene, the Cas protein can cleave the guide RNA target, the nucleic acid construct can be inserted into the ASS1 gene to create a modified ASS1 gene, and ASS1 protein can be expressed from the modified ASS1 gene (e.g., such that a therapeutically effective level of ASS1 protein expression is achieved in the subject).

Treatment refers to any administration or application of a therapeutic for disease or disorder in a subject, and includes inhibiting the disease, arresting its development, relieving one or more symptoms of the disease, curing the disease, or preventing reoccurrence of one or more symptoms of the disease. For example, treatment of citrullinemia type I may comprise alleviating symptoms of citrullinemia type I. In one specific example, a method of preventing or inhibiting hyperammonemia in a subject having citrullinemia type I is provided. Citrullinemia type I is described in detail above and refers to a disorder caused by a missing or defective ASS1 gene or ASS1 polypeptide. The disorder includes conditions that are inherited (e.g., autosomal recessive inheritance) and/or acquired (e.g., caused by a spontaneous mutation in the gene). The defective ASS1 gene or ASS1 polypeptide can result in accumulation of upstream urea cycle metabolites citrulline and ammonia (e.g., hyperammonemia).

In some methods, a therapeutically effective amount of the ASS1 nucleic acid construct or the composition comprising the ASS1 nucleic acid construct or the combination of the ASS1 nucleic acid construct and the nuclease agent (e.g., CRISPR/Cas system) is administered to the subject. A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding.

Therapeutic or pharmaceutical compositions comprising the compositions disclosed herein can be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. See also Powell et al. “Compendium of excipients for parenteral formulations” PDA (1998) J. Pharm. Sci. Technol. 52:238-311.

The compositions disclosed herein may be administered to relieve or prevent or decrease the severity of one or more of the symptoms of ASS1 deficiency or citrullinemia type I. Such symptoms are described in more detail elsewhere herein.

The subject in any of the above methods can be one in need of amelioration or treatment of ASS1 deficiency or citrullinemia type I. The subject in any of the above methods can be from any suitable species, such as a eukaryote or a mammal. A mammal can be, for example, a non-human mammal, a human, a rodent, a rat, a mouse, or a hamster. Other non-human mammals include, for example, non-human primates, monkeys, apes, cats, dogs, rabbits, horses, bulls, deer, bison, livestock (e.g., bovine species such as cows, steer, and so forth; ovine species such as sheep, goats, and so forth; and porcine species such as pigs and boars). Birds include, for example, chickens, turkeys, ostrich, geese, ducks, and so forth. Domesticated animals and agricultural animals are also included. The term “non-human” excludes humans. Specific examples of suitable species include, but are not limited to, humans, rodents, mice, rats, and non-human primates. In a specific example, the subject is a human.

In methods in which an ASS1 nucleic acid construct is genomically integrated, any target genomic locus capable of expressing a gene can be used, such as an endogenous ASS1 locus or a safe harbor locus (safe harbor gene). Such loci are described in more detail elsewhere herein. In a specific example, the target genomic locus can be an endogenous ASS1 locus, such as an endogenous human ASS1 locus. For example, the nucleic acid construct can be genomically integrated in intron 2 of the endogenous human ASS1 locus. Endogenous ASS1 exons 1 and 2 can then splice into the coding sequence for the ASS1 protein in the nucleic acid construct. As another example, the nucleic acid construct can be genomically integrated in intron 1 of the endogenous human ASS1 locus. Endogenous ASS1 exon 1 can then splice into the coding sequence for the ASS1 protein in the nucleic acid construct.

Targeted insertion of the ASS1 nucleic acid construct comprising the ASS1 coding sequence into a target genomic locus, and particularly an endogenous ASS1 locus, offers multiple advantages. Such methods result in stable modification to allow for stable, long-term expression of the ASS1 coding sequence. With respect to the ASS1 locus, such methods are able to utilize the endogenous ASS1 promoter and regulatory regions to achieve therapeutically effective levels of expression. For example, the ASS1 coding sequence in the nucleic acid construct can comprise a promoterless gene, and the inserted nucleic acid construct can be operably linked to an endogenous promoter in the target genomic locus (e.g., ASS1 locus). Use of an endogenous promoter is advantageous because it obviates the need for inclusion of a promoter in the nucleic acid construct, allowing packaging of larger transgenes that may not normally package efficiently (e.g., in AAV). Alternatively, the ASS1 coding sequence in the nucleic acid construct can be operably linked to an exogenous promoter in the nucleic acid construct. Examples of types of promoters that can be used are disclosed elsewhere herein.

Optionally, some or all of the endogenous gene (e.g., endogenous ASS1 gene) at the target genomic locus can be expressed upon insertion of the ASS1 coding sequence from the nucleic acid construct. Alternatively, in some methods, none of the endogenous gene at the target genomic locus is expressed. As one example, the modified target genomic locus (e.g., modified ASS1 locus) after integration of the nucleic acid construct can encode a chimeric protein comprising an endogenous portion of ASS1 and the ASS1 protein encoded by the nucleic acid constructs. In one example, the second intron of an ASS1 locus can be targeted. In such a scenario, a promoterless cassette bearing a splice acceptor and the ASS1 coding sequence (e.g., human ASS1 exons 3-14) will support expression of the ASS1 protein. Splicing between endogenous ASS1 exons 1 and 2 and the integrated ASS1 coding sequence creates a chimeric mRNA and protein including the endogenous ASS1 sequence encoded by exons 1 and 2 operably linked to the ASS1 sequence encoded by the integrated nucleic acid construct.

The ASS1 nucleic acid construct can be inserted into the target genomic locus by any means, including homologous recombination (HR) and non-homologous end joining (NHEJ) as described elsewhere herein. In a specific example, the ASS1 nucleic acid construct is inserted by NHEJ (e.g., does not comprise homology arms and is inserted by NHEJ).

In another specific example, the nucleic acid construct can be inserted via homology-independent targeted integration (e.g., directional homology-independent targeted integration). For example, the ASS1 coding sequence in the nucleic acid construct can be flanked on each side by a target site for a nuclease agent (e.g., the same target site as in the target genomic locus, and the same nuclease agent being used to cleave the target site in the target genomic locus). The nuclease agent can then cleave the target sites flanking the ASS1 coding sequence. In a specific example, the nucleic acid construct is delivered AAV-mediated delivery, and cleavage of the target sites flanking the ASS1 coding sequence can remove the inverted terminal repeats (ITRs) of the AAV. Removal of the ITRs can make it easier to assess successful targeting, because presence of the ITRs can hamper sequencing efforts due to the repeated sequences. In some methods, the target site in the target genomic locus (e.g., a gRNA target sequence including the flanking protospacer adjacent motif) is no longer present if the ASS1 coding sequence is inserted into the target genomic locus in the correct orientation but it is reformed if the ASS1 protein coding sequence is inserted into the target genomic locus in the opposite orientation. This can help ensure that the ASS1 coding sequence is inserted in the correct orientation for expression.

In any of the above methods, the ASS1 nucleic acid construct can be administered simultaneously with the nuclease agent (e.g., CRISPR/Cas system) or not simultaneously (e.g., sequentially in any combination). For example, in a method comprising administering a composition comprising the ASS1 nucleic acid construct and a nuclease agent, they can be administered separately. For example, the ASS1 nucleic acid construct can be administered prior to the nuclease agent, subsequent to the nuclease agent, or at the same time as the nuclease agent. Any suitable methods of administering nucleic acid constructs and nuclease agents to cells can be used, particularly methods of administering to the liver, and examples of such methods are described in more detail elsewhere herein. In methods of treatment or in methods of targeting a cell in vivo in a subject, the nucleic acid construct can be inserted in particular types of cells in the subject. The method and vehicle for introducing the ASS1 nucleic acid construct and/or the nuclease agent into the subject can affect which types of cells in the subject are targeted. In some methods, for example, the nucleic acid construct is inserted into a target genomic locus (e.g., an endogenous ASS1 locus) in liver cells, such as hepatocytes. Methods and vehicles for introducing such constructs and nuclease agents into the subject (including methods and vehicles that target the liver or hepatocytes, such as lipid nanoparticle-mediated delivery and AAV-mediated delivery (e.g., rAAV8-mediated delivery or rAAV5-mediated delivery) and intravenous injection), are disclosed in more detail elsewhere herein.

In methods in which a composition comprising a nucleic acid construct (or vector or LNP) and a nuclease agent is administered (i.e., in methods in which a nucleic acid construct (or vector or LNP) and a nuclease agent are both administered), the nucleic acid construct and the nuclease agent can be administered simultaneously. Alternatively, the nucleic acid construct and the nuclease agent can be administered sequentially in any order. For example, the nucleic acid construct can be administered after the nuclease agent, or the nuclease agent can be administered after the nucleic acid construct. For example, the nuclease agent can be administered about 1 hour to about 48 hours, about 1 hour to about 24 hours, about 1 hour to about 12 hours, about 1 hour to about 6 hours, about 1 hour to about 2 hours, about 2 hours to about 48 hours, about 2 hours to about 24 hours, about 2 hours to about 12 hours, about 2 hours to about 6 hours, about 3 hours to about 48 hours, about 6 hours to about 48 hours, about 12 hours to about 48 hours, or about 24 hours to about 48 hours prior to or subsequent to administration of the nucleic acid construct.

In one example, the nucleic acid construct is administered about 4 hours, about 8 hours, about 12 hours, about 18 hours, about 1 day, about 2 days, about 3 days, about 4 days, about 5 days, about 6 days, or about 1 week prior to administering the nuclease agent. In another example, the nucleic acid construct is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to administering the nuclease agent. In another example, the nucleic acid construct is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to administering the nuclease agent.

In one example, the nucleic acid construct is administered about 4 hours, about 8 hours, about 12 hours, about 18 hours, about 1 day, about 2 days, about 3 days, about 4 days, about 5 days, about 6 days, or about 1 week after administering the nuclease agent. In another example, the nucleic acid construct is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week after administering the nuclease agent. In another example, the nucleic acid construct is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days after administering the nuclease agent.

In any of the above methods, the ASS1 nucleic acid construct and the nuclease agent (e.g., CRISPR/Cas system) can be administered using any suitable delivery system and known method. The nuclease agent components and ASS1 nucleic acid construct (e.g., the guide RNA, Cas protein, and ASS1 nucleic acid construct) can be delivered individually or together in any combination, using the same or different delivery methods as appropriate.

In methods in which a CRISPR/Cas system is used, a guide RNA can be introduced into or administered to a subject or cell, for example, in the form of an RNA (e.g., in vitro transcribed RNA, such as the modified guide RNAs disclosed herein) or in the form of a DNA encoding the guide RNA. When introduced in the form of a DNA, the DNA encoding a guide RNA can be operably linked to a promoter active in the cell or in a cell in the subject. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be introduced into a subject or cell in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)), such as a modified mRNA as disclosed herein, or DNA). Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or a subject, the Cas protein can be transiently, conditionally, or constitutively expressed in the cell or in a cell in the subject.

In one example, the Cas protein is introduced in the form of an mRNA (e.g., a modified mRNA as disclosed herein), and the guide RNA is introduced in the form of RNA such as a modified gRNA as disclosed herein (e.g., together within the same lipid nanoparticle). Guide RNAs can be modified as disclosed elsewhere herein. Likewise, Cas mRNAs can be modified as disclosed elsewhere herein.

In methods in which an ASS1 nucleic acid construct is inserted following cleavage by a gene-editing system (e.g., a Cas protein), the gene-editing system (e.g., Cas protein) can cleave the target genomic locus to create a single-strand break (nick) or double-strand break, and the cleaved or nicked locus can be repaired by insertion of the ASS1 nucleic acid construct via non-homologous end joining (NHEJ)-mediated insertion or homology-directed repair. Optionally, repair with the ASS1 nucleic acid construct removes or disrupts the guide RNA target sequence(s) so that alleles that have been targeted cannot be re-targeted by the CRISPR/Cas reagents.

As explained in more detail elsewhere herein, the ASS1 nucleic acid constructs can comprise deoxyribonucleic acid (DNA) or ribonucleic acid (RNA), they can be single-stranded or double-stranded, and they can be in linear or circular form. The ASS1 nucleic acid constructs can be naked nucleic acids or can be delivered by viruses, such as AAV. In a specific example, the ASS1 nucleic acid construct can be delivered via AAV and can be capable of insertion into the target genomic locus (e.g., an endogenous gene, an ASS1 gene, or intron 2 of a human ASS1 gene, or intron 1 of a human ASS1 gene) by non-homologous end joining (e.g., the ASS1 nucleic acid construct can be one that does not comprise homology arms).

Some ASS1 nucleic acid constructs are capable of insertion by non-homologous end joining. In some cases, such ASS1 nucleic acid constructs do not comprise homology arms. For example, such ASS1 nucleic acid constructs can be inserted into a blunt end double-strand break following cleavage with a Cas protein. In a specific example, the ASS1 nucleic acid construct can be delivered via AAV and can be capable of insertion by non-homologous end joining (e.g., the ASS1 nucleic acid construct can be one that does not comprise homology arms).

In another example, the ASS1 nucleic acid construct can be inserted via homology-independent targeted integration. For example, the ASS1 nucleic acid construct can be flanked on each side by a guide RNA target sequence (e.g., the same target site as in the target genomic locus, and the CRISPR/Cas reagent (Cas protein and guide RNA) being used to cleave the target site in the target genomic locus). The Cas protein can then cleave the target sites flanking the nucleic acid insert. In a specific example, the ASS1 nucleic acid construct is delivered AAV-mediated delivery, and cleavage of the target sites flanking the nucleic acid insert can remove the inverted terminal repeats (ITRs) of the AAV. In some methods, the target site in the target genomic locus (e.g., a guide RNA target sequence including the flanking protospacer adjacent motif) is no longer present if the nucleic acid insert is inserted into the target genomic locus in the correct orientation but it is reformed if the nucleic acid insert is inserted into the target genomic locus in the opposite orientation.

The methods disclosed herein can comprise introducing or administering into a subject (e.g., an animal or mammal, such as a human) or cell an ASS1 nucleic acid construct and optionally a nuclease agent such as CRISPR/Cas reagents, including in the form of nucleic acids (e.g., DNA or RNA), proteins, or nucleic-acid-protein complexes. “Introducing” or “administering” includes presenting to the cell or subject the molecule(s) (e.g., nucleic acid(s) or protein(s)) in such a manner that it gains access to the interior of the cell or to the interior of cells within the subject. The introducing can be accomplished by any means, and two or more of the components (e.g., two of the components, or all of the components) can be introduced into the cell or subject simultaneously or sequentially in any combination. For example, a Cas protein can be introduced into a cell or subject before introduction of a guide RNA, or it can be introduced following introduction of the guide RNA. As another example, an ASS1 nucleic acid construct can be introduced prior to the introduction of a Cas protein and a guide RNA, or it can be introduced following introduction of the Cas protein and the guide RNA (e.g., the ASS1 nucleic acid construct can be administered about 1, 2, 3, 4, 8, 12, 24, 36, 48, or 72 hours before or after introduction of the Cas protein and the guide RNA). See, e.g., US 2015/0240263 and US 2015/0110762, each of which is herein incorporated by reference in its entirety for all purposes. In addition, two or more of the components can be introduced into the cell or subject by the same delivery method or different delivery methods. Similarly, two or more of the components can be introduced into a subject by the same route of administration or different routes of administration.

A guide RNA can be introduced into a subject or cell, for example, in the form of an RNA (e.g., in vitro transcribed RNA) or in the form of a DNA encoding the guide RNA. Guide RNAs can be modified as disclosed elsewhere herein. When introduced in the form of a DNA, the DNA encoding a guide RNA can be operably linked to a promoter active in the cell or in a cell in the subject. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Cas RNAs can be modified as disclosed elsewhere herein. Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or a subject, the Cas protein can be transiently, conditionally, or constitutively expressed in the cell or in a cell in the subject.

Nucleic acids encoding Cas proteins or guide RNAs can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding one or more gRNAs. Alternatively, it can be in a vector or plasmid that is separate from the vector comprising the DNA encoding one or more gRNAs. Suitable promoters that can be used in an expression construct include promoters active, for example, in one or more of a eukaryotic cell, a human cell, a non-human cell, a mammalian cell, a non-human mammalian cell, a rodent cell, a mouse cell, a rat cell, a hamster cell, a rabbit cell, a pluripotent cell, an embryonic stem (ES) cell, an adult stem cell, a developmentally restricted progenitor cell, an induced pluripotent stem (iPS) cell, or a one-cell stage embryo. For example, a suitable promoter can be active in a liver cell such as a hepatocyte. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allows for the generation of compact expression cassettes to facilitate delivery.

Molecules (e.g., Cas proteins or guide RNAs or nucleic acids encoding) introduced into the subject or cell can be provided in compositions comprising a carrier increasing the stability of the introduced molecules (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules.

Various methods and compositions are provided herein to allow for introduction of molecule (e.g., a nucleic acid or protein) into a cell or subject. Methods for introducing molecules into various cell types are known and include, for example, stable transfection methods, transient transfection methods, and virus-mediated methods.

Transfection protocols as well as protocols for introducing molecules into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4):1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of nucleic acids or proteins into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of molecules (e.g., nucleic acids or proteins) into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Microinjection of an mRNA is preferably into the cytoplasm (e.g., to deliver mRNA directly to the translation machinery), while microinjection of a Cas protein or a polynucleotide encoding a Cas protein or encoding an RNA is preferable into the nucleus/pronucleus. Alternatively, microinjection can be carried out by injection into both the nucleus/pronucleus and the cytoplasm: a needle can first be introduced into the nucleus/pronucleus and a first amount can be injected, and while removing the needle from the one-cell stage embryo a second amount can be injected into the cytoplasm. If a Cas protein is injected into the cytoplasm, the Cas protein preferably comprises a nuclear localization signal to ensure delivery to the nucleus/pronucleus. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); see also Meyer et al. (2010) Proc. Natl. Acad. Sci. U.S.A. 107:15022-15026 and Meyer et al. (2012) Proc. Natl. Acad. Sci. U.S.A. 109:9354-9359, each of which is herein incorporated by reference in its entirety for all purposes.

Other methods for introducing molecules (e.g., nucleic acid or proteins) into a cell or subject can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a cell or subject in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a subject include hydrodynamic delivery, virus-mediated delivery (e.g., adeno-associated virus (AAV)-mediated delivery), and lipid-nanoparticle-mediated delivery.

Introduction of nucleic acids and proteins into cells or subjects can be accomplished by hydrodynamic delivery (HDD). For gene delivery to parenchymal cells, only essential DNA sequences need to be injected via a selected blood vessel, eliminating safety concerns associated with current viral and synthetic vectors. When injected into the bloodstream, DNA is capable of reaching cells in the different tissues accessible to the blood. Hydrodynamic delivery employs the force generated by the rapid injection of a large volume of solution into the incompressible blood in the circulation to overcome the physical barriers of endothelium and cell membranes that prevent large and membrane-impermeable compounds from entering parenchymal cells. In addition to the delivery of DNA, this method is useful for the efficient intracellular delivery of RNA, proteins, and other small compounds in vivo. See, e.g., Bonamassa et al. (2011) Pharm. Res. 28(4):694-701, herein incorporated by reference in its entirety for all purposes.

Introduction of nucleic acids can also be accomplished by virus-mediated delivery, such as AAV-mediated delivery or lentivirus-mediated delivery. Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression (e.g., of Cas9 and/or gRNA). Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹², about 10¹³, about 10¹⁴, about 10¹⁵, and about 10¹⁶ vector genomes (vg)/mL, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/mL. Other exemplary viral titers (e.g., AAV titers) include about 10¹², about 10¹³, about 10¹⁴, about 10¹⁵, and about 10¹⁶ vector genomes (vg)/kg of body weight, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/kg of body weight. In one example, the viral titer is between about 10¹³ to about 10¹⁴ vg/mL or vg/kg. In another example, the viral titer is between about 10¹² to about 10¹³ vg/mL or vg/kg (e.g., between about 10¹² to about 10¹³ vg/kg). In another example, the viral titer is between about 10¹² to about 10¹⁴ vg/mL or vg/kg (e.g., between about 10¹² to about 10¹⁴ vg/kg). For example, the viral titer can be between about 1.5E12 toa bout 1.5E13 vg/kg, can be about 1.5E12 vg/kg, or can be about 1.5E13 vg/kg. AAVs for use in the methods are discussed in more detail elsewhere herein.

Introduction of nucleic acids and proteins can also be accomplished by lipid nanoparticle (LNP)-mediated delivery. For example, LNP-mediated delivery can be used to deliver a combination of Cas mRNA and guide RNA or a combination of Cas protein and guide RNA. LNP-mediated delivery can be used to deliver a guide RNA in the form of RNA. In a specific example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. As another example, Cas mRNA modifications can include substitution with N1-methyl-pseudouridine (e.g., fully substituted with N1-methyl-pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include an ASS1 nucleic acid construct. In certain LNPs, the cargo can include an mRNA encoding a Cas nuclease, such as Cas9, a guide RNA or a nucleic acid encoding a guide RNA, and an ASS1 nucleic acid construct. LNPs for use in the methods are described in more detail elsewhere herein.

Exemplary dosing of LNPs includes about 0.1, about 0.25, about 0.3, about 0.5, about 1, about 2, about 3, about 4, about 5, about 6, about 8, or about 10 mg/kg body weight (mpk) or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 1, about 3, or about 10 mg/kg can be used. Additional exemplary dosing of LNPs includes about 0.1, about 0.25, about 0.3, about 0.5, about 1, about 2, about 3, about 4, about 5, about 6, about 8, or about 10 mg/kg (mpk) body weight or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 0.5, about 1, about 2, about 3, or about 10 mg/kg can be used. In another example, LNP doses between about 0.5 and about 10, between about 0.5 and about 5, between about 0.5 and about 3, between about 1 and about 10, between about 1 and about 5, between about 1 and about 3, or between about 1 and about 2 mg/kg can be used. In another example, LNP doses between about 0.5 and about 3, between about 0.5 and about 2.5, between about 0.5 and about 2, between about 0.5 and about 1.5, between about 0.5 and about 1, between about 1 and about 3, between about 1 and about 2.5, between about 1 and about 2, or between about 1 and about 1.5 mg/kg can be used. In another example, an LNP dose of about 1 mg/kg can be used.

The mode of delivery can be selected to decrease immunogenicity. For example, a Cas protein and a gRNA may be delivered by different modes (e.g., bi-modal delivery). These different modes may confer different pharmacodynamics or pharmacokinetic properties on the subject delivered molecule (e.g., Cas or nucleic acid encoding, gRNA or nucleic acid encoding, or ASS1 nucleic acid construct). For example, the different modes can result in different tissue distribution, different half-life, or different temporal distribution. Some modes of delivery (e.g., delivery of a nucleic acid vector that persists in a cell by autonomous replication or genomic integration) result in more persistent expression and presence of the molecule, whereas other modes of delivery are transient and less persistent (e.g., delivery of an RNA or a protein). Delivery of Cas proteins in a more transient manner, for example, as mRNA or protein, can ensure that the Cas/gRNA complex is only present and active for a short period of time and can reduce immunogenicity caused by peptides from the bacterially-derived Cas enzyme being displayed on the surface of the cell by MHC molecules. Such transient delivery can also reduce the possibility of off-target modifications.

Administration in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically. In a specific example, administration in vivo is intravenous.

Administration in vivo can be by any suitable route including, for example, parenteral, intravenous, oral, subcutaneous, intra-arterial, intracranial, intrathecal, intraperitoneal, topical, intranasal, or intramuscular. A specific example is intravenous infusion. Compositions comprising the guide RNAs and/or Cas proteins (or nucleic acids encoding the guide RNAs and/or Cas proteins) can be formulated using one or more physiologically and pharmaceutically acceptable carriers, diluents, excipients or auxiliaries. The formulation can depend on the route of administration chosen. Pharmaceutically acceptable means that the carrier, diluent, excipient, or auxiliary is compatible with the other ingredients of the formulation and not substantially deleterious to the recipient thereof. In a specific example, the route of administration and/or formulation or chosen for delivery to the liver (e.g., hepatocytes).

The frequency of administration and the number of dosages can depend on a number of factors. The introduction of nucleic acids or proteins into the cell or subject can be performed one time or multiple times over a period of time. For example, the introduction can be performed only once over a period of time, at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time. In some methods, a single administration of the ASS1 nucleic acid construct (or a single administration of the ASS1 nucleic acid construct and nuclease agent (e.g., Cas protein and guide RNA)) is sufficient to increase expression of ASS1 to a desirable level. In other methods, more than one administration may be beneficial to maximize therapeutic effect.

The methods disclosed herein can increase ASS1 protein levels and/or ASS1 activity levels in a cell or subject and can comprise measuring urea cycle metabolites upstream of ASS1 (e.g., serum or plasma ammonia and/or citrulline levels in a subject) or phenotypic characteristics related to ASS1 protein levels and/or ASS1 activity (e.g., body weight of a subject). In one example, the effectiveness of the treatment in a subject can be assessed by measuring serum or plasma ammonia and/or citrulline levels, wherein a decrease in ammonia and/or citrulline levels indicates effectiveness of the treatment. In another example, effectiveness of the treatment in a subject can be assessed by measuring the body weight of a subject, wherein an increase in body weight indicates effectiveness of the treatment.

In normal or healthy newborns within one month of age, serum or plasma ammonia levels vary between about 21-95 μmol/L, with a single upper reference limit of 82 μmol/L for all newborns less than 1 week of age having been proposed. See, e.g., Ni et al. (2022) Medicine (Baltimore) 101(48): e31796, herein incorporated by reference in its entirety for all purposes. For adult individuals, serum or plasma ammonia levels are considered normal if less than about 60 μmol/L. See, e.g., Quinonez and Lee, Citrullinemia Type I (2004) [Updated 2022 Aug. 18], In: Adam et al. eds. GeneReviews® [Internet], Seattle (WA): University of Washington, Seattle; 1993-2024, herein incorporated by reference in its entirety for all purposes. Regardless of subject age, pathologically elevated serum or plasma ammonia is considered to be >150 μmol/L. Id. In a specific example, normal serum or plasma ammonia levels are considered to be less than the upper limit of normal (ULN) of about 60 μmol/L. In a specific example, pathologic serum or plasma ammonia levels are considered to be greater than about 1.5 times the ULN or greater than 1.5 times the ULN.

In some methods, serum or plasma ammonia levels in a subject having citrullinemia type I or ASS1 deficiency are reduced to be less than about or about 1.5 times, less than about or about 1.4 times, less than about or about 1.3 times, less than about or about 1.2 times, less than about or about 1.1 times, less than about or about 1.0 times, less than about or about 0.9 times, less than about or about 0.8 times, less than about or about 0.7 times, less than about or about 0.6 times, less than about or about 0.5 times, less than about or about 0.4 times, less than about or about 0.3 times, less than about or about 0.2 times, or less than about or about 0.1 times the ULN.

In some methods, serum or plasma ammonia levels in a subject having citrullinemia type I or ASS1 deficiency are reduced to be between about 0.1 to about 1.0 times the ULN, between about 0.2 to about 1.0 times the ULN, between about 0.3 to about 1.0 times the ULN, between about 0.4 to about 1.0 times the ULN, between about 0.5 to about 1.0 times the ULN, between about 0.6 to about 1.0 times the ULN, between about 0.7 to about 1.0 times the ULN, between about 0.8 to about 1.0 times the ULN, between about 0.9 to about 1.5 times the ULN, between about 1.0 to about 1.5 times the ULN, between about 1.1 to about 1.5 times the ULN, between about 1.2 to about 1.5 times the ULN, between about 1.3 to about 1.5 times the ULN, between about 0.1 to about 0.5 times the ULN, between about 0.2 to about 0.6 times the ULN, between about 0.3 to about 0.7 times the ULN, or between about 0.4 to about 0.8 times the ULN.

In some methods, serum or plasma ammonia levels in a subject having citrullinemia type I or ASS1 deficiency are reduced to be less than about or about 150 μmol/L, less than about or about 145 μmol/L, less than about or about 140 μmol/L, less than about or about 135 μmol/L, less than about or about 130 μmol/L, less than about or about 125 μmol/L, less than about or about 120 μmol/L, less than about or about 115 μmol/L, less than about or about 110 μmol/L, less than about or about 105 μmol/L, less than about or about 100 μmol/L, less than about or about 95 μmol/L, less than about or about 90 μmol/L, less than about or about 85 μmol/L, less than about or about 80 μmol/L, less than about or about 75 μmol/L, less than about or about 70 μmol/L, less than about or about 65 μmol/L, less than about or about 60 μmol/L, less than about or about 55 μmol/L, less than about or about 50 μmol/L, less than about or about 45 μmol/L, less than about or about 40 μmol/L, less than about or about 35 μmol/L, less than about or about 30 μmol/L, less than about or about 25 μmol/L, less than about or about 20 μmol/L, less than about or about 15 μmol/L, or less than about or about 10 μmol/L. In one specific example, serum or plasma ammonia levels in a subject having citrullinemia type I or ASS1 deficiency are reduced to be less than about or about 150 μmol/L. In another specific example, serum or plasma ammonia levels in a subject having citrullinemia type I or ASS1 deficiency are reduced to be less than about or about 100 μmol/L.

In some methods, serum or plasma ammonia levels in a subject having citrullinemia type I or ASS1 deficiency are reduced to be between about 10 μmol/L to about 100 μmol/L, between about 15 μmol/L to about 100 μmol/L, between about 20 μmol/L to about 100 μmol/L, between about 25 μmol/L to about 100 μmol/L, between about 30 μmol/L to about 100 μmol/L, between about 35 μmol/L to about 100 μmol/L, between about 40 μmol/L to about 100 μmol/L, between about 45 μmol/L to about 100 μmol/L, between about 50 μmol/L to about 100 μmol/L, between about 55 μmol/L to about 100 μmol/L, between about 60 μmol/L to about 100 μmol/L, between about 65 μmol/L to about 100 μmol/L, between about 70 μmol/L to about 100 μmol/L, between about 75 μmol/L to about 100 μmol/L, between about 80 μmol/L to about 100 μmol/L, between about 85 μmol/L to about 100 μmol/L, between about 90 μmol/L to about 100 μmol/L. between about 10 μmol/L to about 50 μmol/L, between about 15 μmol/L to about 50 μmol/L, between about 20 μmol/L to about 50 μmol/L, between about 25 μmol/L to about 50 μmol/L, between about 30 μmol/L to about 50 μmol/L, between about 35 μmol/L to about 50 μmol/L, or between about 40 μmol/L to about 50 μmol/L.

In some embodiments, the recited serum or plasma ammonia levels are at least 2 weeks after administration. In some embodiments, the recited serum or plasma ammonia levels are at least 4 weeks after administration. In some embodiments, the recited serum or plasma ammonia levels are at least 6 weeks after administration. In some embodiments, the recited serum or plasma ammonia levels are at least 8 weeks after administration. In some embodiments, the recited serum or plasma ammonia levels are at least 12 weeks after administration. In some embodiments, the recited serum or plasma ammonia levels are at least 16 weeks after administration. In some embodiments, the recited serum or plasma ammonia levels are at least 20 weeks after administration. In some embodiments, the recited serum or plasma ammonia levels are at least 1 month after administration. In some embodiments, the recited serum or plasma ammonia levels are at least 2 months after administration. In some embodiments, the recited serum or plasma ammonia levels are at least 3 months after administration. In some embodiments, the recited serum or plasma ammonia levels are at least 4 months after administration. In some embodiments, the recited serum or plasma ammonia levels are at least 5 months after administration. In some embodiments, the recited serum or plasma ammonia levels are at least 6 months after administration. In some embodiments, the recited serum or plasma ammonia levels are at least 9 months after administration. In some embodiments, the recited serum or plasma ammonia levels are at least 1 year after administration. In some embodiments, the recited serum or plasma ammonia levels are at least 2 years after administration. In some embodiments, the recited serum or plasma ammonia levels are at 2 weeks after administration. In some embodiments, the recited serum or plasma ammonia levels are at 4 weeks after administration. In some embodiments, the recited serum or plasma ammonia levels are at 6 weeks after administration. In some embodiments, the recited serum or plasma ammonia levels are at 8 weeks after administration. In some embodiments, the recited serum or plasma ammonia levels are at 12 weeks after administration. In some embodiments, the recited serum or plasma ammonia levels are at 16 weeks after administration. In some embodiments, the recited serum or plasma ammonia levels are at 20 weeks after administration. In some embodiments, the recited serum or plasma ammonia levels are at 1 month after administration. In some embodiments, the recited serum or plasma ammonia levels are at 2 months after administration. In some embodiments, the recited serum or plasma ammonia levels are at 3 months after administration. In some embodiments, the recited serum or plasma ammonia levels are at 4 months after administration. In some embodiments, the recited serum or plasma ammonia levels are at 5 months after administration. In some embodiments, the recited serum or plasma ammonia levels are at 6 months after administration. In some embodiments, the recited serum or plasma ammonia levels are at 9 months after administration. In some embodiments, the recited serum or plasma ammonia levels are at 1 year after administration. In some embodiments, the recited serum or plasma ammonia levels are at 2 years after administration.

Serum or plasma citrulline concentrations in normal or healthy individuals have been defined as 40 (±10) μmol/L. See, e.g., Maric et al. (2021) Nutrients. 13(8):2794, herein incorporated by reference in its entirety for all purposes. Serum or plasma citrulline levels in subjects having citrullinemia type I or ASS1 deficiency are typically greater than about 500 μmol/L. See, e.g., Quinonez and Lee, supra. In a specific example, normal serum or plasma citrulline levels are considered to be less than the upper limit of normal (ULN) of about 50 μmol/L or 50 μmol/L. In a specific example, pathologic serum or plasma citrulline levels are considered to be greater than about 10 times the ULN or greater than 10 times the ULN.

In some methods, serum or plasma citrulline levels in a subject having citrullinemia type I or ASS1 deficiency are reduced to be less than about or about 10 times, less than about or about 9.5 times, less than about or about 9.0 times, less than about or about 8.5 times, less than about or about 8.0 times, less than about or about 7.5 times, less than about or about 7.0 times, less than about or about 6.5 times, less than about or about 6.0 times, less than about or about 5.5 times, less than about or about 5.0 times, less than about or about 4.5 times, less than about or about 4.0 times, less than about or about 3.5 times, less than about or about 3.0 times, less than about or about 2.5 times, less than about or about 2.0 times, less than about or about 1.5 times, less than about or about 1.4 times, less than about or about 1.3 times, less than about or about 1.2 times, less than about or about 1.1 times, less than about or about 1.0 times, less than about or about 0.9 times, less than about or about 0.8 times, less than about or about 0.7 times, less than about or about 0.6 times, or less than about or about 0.5 times the ULN.

In some methods, serum or plasma citrulline levels in a subject having citrullinemia type I or ASS1 deficiency are reduced to be between about 0.5 to about 5.0 times the ULN, between about 1.0 to about 5.0 times the ULN, between about 1.5 to about 5.0 times the ULN, between about 2.0 to about 5.0 times the ULN, between about 2.5 to about 5.0 times the ULN, between about 3.0 to about 5.0 times the ULN, between about 3.5 to about 5.0 times the ULN, between about 4.0 to about 5.0 times the ULN, between about 4.5 to about 5.0 times the ULN, between about 5.0 to about 10.0 times the ULN, between about 5.5 to about 10.0 times the ULN, between about 6.0 to about 10.0 times the ULN, between about 6.5 to about 10.0 times the ULN, between about 7.0 to about 10.0 times the ULN, between about 7.5 to about 10.0 times the ULN, between about 8.0 to about 10.0 times the ULN, between about 8.5 to about 10.0 times the ULN, between about 9.0 to about 10.0 times the ULN, between about 9.5 to about 10.0 times the ULN, between about 0.5 to about 1.0 times the ULN, between about 0.6 to about 1.0 times the ULN, between about 0.7 to about 1.0 times the ULN, between about 0.8 to about 1.0 times the ULN, between about 0.8 to about 1.5 times the ULN, between about 0.9 to about 1.5 times the ULN, between about 1.0 to about 1.5 times the ULN, between about 1.2 to about 1.5 times the ULN, or between about 1.3 to about 1.5 times the ULN.

In some methods, serum or plasma citrulline levels in a subject having citrullinemia type I or ASS1 deficiency are reduced to be less than about or about 2000 μmol/L, less than about or about 1750 μmol/L, less than about or about 1500 μmol/L, less than about or about 1250 μmol/L, less than about or about 1000 μmol/L, less than about or about 900 μmol/L, less than about or about 800 μmol/L, less than about or about 700 μmol/L, less than about or about 600 μmol/L, less than about or about 500 μmol/L, less than about or about 450 μmol/L, less than about or about 400 μmol/L, less than about or about 350 μmol/L, less than about or about 300 μmol/L, less than about or about 250 μmol/L, less than about or about 200 μmol/L, less than about or about 150 μmol/L, less than about or about 140 μmol/L, less than about or about 130 μmol/L, less than about or about 120 μmol/L, less than about or about 110 μmol/L, less than about or about 100 μmol/L, less than about or about 90 μmol/L, less than about or about 80 μmol/L, less than about or about 75 μmol/L, less than about or about 70 μmol/L, less than about or about 65 μmol/L, less than about or about 60 μmol/L, less than about or about 55 μmol/L, less than about or about 50 μmol/L, less than about or about 45 μmol/L, less than about or about 40 μmol/L, less than about or about 35 μmol/L, or less than about or about 30 μmol/L. In a specific example, serum or plasma citrulline levels in a subject having citrullinemia type I or ASS1 deficiency are reduced to be less than about or about 500 μmol/L

In some methods, serum or plasma citrulline levels in a subject having citrullinemia type I or ASS1 deficiency are reduced to be between about 30 μmol/L to about 100 μmol/L, between about 40 μmol/L to about 100 μmol/L, between about 50 μmol/L to about 100 μmol/L, between about 60 μmol/L to about 100 μmol/L, between about 70 μmol/L to about 100 μmol/L, between about 80 μmol/L to about 100 μmol/L, between about 90 μmol/L to about 100 μmol/L, between about 100 μmol/L to about 500 μmol/L, between about 150 μmol/L to about 500 μmol/L, between about 200 μmol/L to about 500 μmol/L, between about 250 μmol/L to about 500 μmol/L, between about 300 μmol/L to about 500 μmol/L, between about 350 μmol/L to about 500 μmol/L, between about 400 μmol/L to about 500 μmol/L, between about 450 μmol/L to about 500 μmol/L, between about 25 μmol/L to about 50 μmol/L, between about 30 μmol/L to about 50 μmol/L, between about 35 μmol/L to about 50 μmol/L, between about 40 μmol/L to about 50 μmol/L, or between about 45 μmol/L to about 50 μmol/L.

In some embodiments, the recited serum or plasma citrulline levels are at least 2 weeks after administration. In some embodiments, the recited serum or plasma citrulline levels are at least 4 weeks after administration. In some embodiments, the recited serum or plasma citrulline levels are at least 6 weeks after administration. In some embodiments, the recited serum or plasma citrulline levels are at least 8 weeks after administration. In some embodiments, the recited serum or plasma citrulline levels are at least 12 weeks after administration. In some embodiments, the recited serum or plasma citrulline levels are at least 16 weeks after administration. In some embodiments, the recited serum or plasma citrulline levels are at least 20 weeks after administration. In some embodiments, the recited serum or plasma citrulline levels are at least 1 month after administration. In some embodiments, the recited serum or plasma citrulline levels are at least 2 months after administration. In some embodiments, the recited serum or plasma citrulline levels are at least 3 months after administration. In some embodiments, the recited serum or plasma citrulline levels are at least 4 months after administration. In some embodiments, the recited serum or plasma citrulline levels are at least 5 months after administration. In some embodiments, the recited serum or plasma citrulline levels are at least 6 months after administration. In some embodiments, the recited serum or plasma citrulline levels are at least 9 months after administration. In some embodiments, the recited serum or plasma citrulline levels are at least 1 year after administration. In some embodiments, the recited serum or plasma citrulline levels are at least 2 years after administration. In some embodiments, the recited serum or plasma citrulline levels are at 2 weeks after administration. In some embodiments, the recited serum or plasma citrulline levels are at 4 weeks after administration. In some embodiments, the recited serum or plasma citrulline levels are at 6 weeks after administration. In some embodiments, the recited serum or plasma citrulline levels are at 8 weeks after administration. In some embodiments, the recited serum or plasma citrulline levels are at 12 weeks after administration. In some embodiments, the recited serum or plasma citrulline levels are at 16 weeks after administration. In some embodiments, the recited serum or plasma citrulline levels are at 20 weeks after administration. In some embodiments, the recited serum or plasma citrulline levels are at 1 month after administration. In some embodiments, the recited serum or plasma citrulline levels are at 2 months after administration. In some embodiments, the recited serum or plasma citrulline levels are at 3 months after administration. In some embodiments, the recited serum or plasma citrulline levels are at 4 months after administration. In some embodiments, the recited serum or plasma citrulline levels are at 5 months after administration. In some embodiments, the recited serum or plasma citrulline levels are at 6 months after administration. In some embodiments, the recited serum or plasma citrulline levels are at 9 months after administration. In some embodiments, the recited serum or plasma citrulline levels are at 1 year after administration. In some embodiments, the recited serum or plasma citrulline levels are at 2 years after administration.

The Centers for Disease Control and Prevention (CDC), National Center for Health Statistics provides World Health Organization (WHO) and CDC growth charts that describe percentile distributions of length-for-age and weight-for-age in male and female populations (accessible from: cdc.gov/growthcharts/who-charts). Children with nutritional deficiency or other genetic conditions (e.g., metabolic genetic conditions, including citrullinemia type I or ASS1 deficiency) can be considered to be abnormally underweight (e.g., exhibiting failure to thrive or FTT) when their weight is below the 5^{th} percentile for sex and corrected age. See, e.g., Homan (2016) Am. Fam. Physician 94(4):295-9. In a specific example, a subject is considered to have a normal body weight when corresponding to the 50^{th} percentile for sex and corrected age according to the WHO and/or CDC growth charts.

In some methods, the body weight of a subject having citrullinemia type I or ASS1 deficiency is increased to about or to at least about 10%, about or to at least about 20%, about or to at least about 30%, about or to at least about 40%, about or to at least about 50%, about or to at least about 60%, about or to at least about 70%, about or to at least about 75%, about or to at least about 80%, about or to at least about 85%, about or to at least about 90%, about or to at least about 95%, about or to at least about 100%, about or to at least about 105%, about or to at least about 110%, about or to at least about 115%, about or to at least about 120%, about or to at least about 125%, about or to at least about 130%, about or to at least about 140%, about or to at least about 150%, about or to at least about 160%, about or to at least about 170%, about or to at least about 180%, about or to at least about 190%, about or to at least about 200% of normal body weight.

In some methods, the body weight of a subject having citrullinemia type I or ASS1 deficiency is increased to between about 10% to about 50% of normal, between about 20% to about 50% of normal, between about 30% to about 50% of normal, between about 50% to about 100% of normal, between about 60% to about 100% of normal, between about 70% to about 100% of normal, between about 80% to about 100% of normal, between about 10% to about 25% of normal, between about 25% to about 50% of normal, between about 50% to about 75% of normal, between about 50% to about 150% of normal, between about 75% to about 125% of normal, between about 90% to about 110% of normal, between about 100% to about 120% of normal, between about 100% to about 150% of normal, between about 100% to about 175% of normal, between about 100% to about 200% of normal, between about 125% to about 150% of normal, between about 150% to about 175% of normal, or between about 150% to about 200% of normal.

In some embodiments, the recited body weights are at least 2 weeks after administration. In some embodiments, the recited body weights are at least 4 weeks after administration. In some embodiments, the recited body weights are at least 6 weeks after administration. In some embodiments, the recited body weights are at least 8 weeks after administration. In some embodiments, the recited body weights are at least 12 weeks after administration. In some embodiments, the recited body weights are at least 16 weeks after administration. In some embodiments, the recited body weights are at least 20 weeks after administration. In some embodiments, the recited body weights are at least 1 month after administration. In some embodiments, the recited body weights are at least 2 months after administration. In some embodiments, the recited body weights are at least 3 months after administration. In some embodiments, the recited body weights are at least 4 months after administration. In some embodiments, the recited body weights are at least 5 months after administration. In some embodiments, the recited body weights are at least 6 months after administration. In some embodiments, the recited body weights are at least 9 months after administration. In some embodiments, the recited body weights are at least 1 year after administration. In some embodiments, the recited body weights are at least 2 years after administration. In some embodiments, the recited body weights are at 2 weeks after administration. In some embodiments, the recited body weights are at 4 weeks after administration. In some embodiments, the recited body weights are at 6 weeks after administration. In some embodiments, the recited body weights are at 8 weeks after administration. In some embodiments, the recited body weights are at 12 weeks after administration. In some embodiments, the recited body weights are at 16 weeks after administration. In some embodiments, the recited body weights are at 20 weeks after administration. In some embodiments, the recited body weights are at 1 month after administration. In some embodiments, the recited body weights are at 2 months after administration. In some embodiments, the recited body weights are at 3 months after administration. In some embodiments, the recited body weights are at 4 months after administration. In some embodiments, the recited body weights are at 5 months after administration. In some embodiments, the recited body weights are at 6 months after administration. In some embodiments, the recited body weights are at 9 months after administration. In some embodiments, the recited body weights are at 1 year after administration. In some embodiments, the recited body weights are at 2 years after administration.

Some methods comprise achieving a durable effect, such as an at least 1 month, at least 2 months, at least 6 months, at least 1 year, or at least 2 year effect. Some methods comprise achieving the therapeutic effect in a durable and sustained manner, such as an at least 1 month, at least 2 months, at least 6 months, at least 1 year, or at least 2 year effect. In some methods, the increased ASS1 activity and/or expression level is stable for at least 1 month, at least 2 months, at least 6 months, at least 1 year, or more. In some methods, a steady-state activity and/or level of ASS1 protein is achieved by at least 7 days, at least 14 days, or at least 28 days. In additional methods, the method comprises maintaining ASS1 activity and/or levels after a single dose for at least 1, at least 2, at least 4, or at least 6 months, or at least 1, at least 2, at least 3, at least 4, or at least 5 years. Some methods comprise achieving a durable or sustained effect in a human, such as an at least at least 8 weeks, at least 24 weeks, for example, at least 1 year (52 weeks), or optionally at least 2 year effect, and in some embodiments, at least 3 year, at least 4 year, or at least 5 year effect. Some methods comprise achieving the therapeutic effect in a human in a durable and sustained manner, such as an at least 8 weeks, at least 24 weeks, for example, at least 1 year, or optionally at least 2 year effect, and in some embodiments, at least 3 year, at least 4 year, or at least 5 year effect. In some methods, the increased ASS1 activity and/or expression level in a human is stable for at least at least 8 weeks, at least 24 weeks, for example, at least 1 year, optionally at least 2 years, and in some embodiments, at least 3 years, at least 4 years, or at least 5 years. In some methods, a steady-state activity and/or level of ASS1 in a human is achieved by at least 7 days, at least 14 days, or at least 28 days, optionally at least 56 days, at least 80 days, or at least 96 days. In additional methods, the method comprises maintaining ASS1 activity and/or levels after a single dose in a human for at least 8 weeks, at least 16 weeks, or at least 24 weeks, or in some embodiments at least 1 year, or at least 2 years, optionally at least 3 years, at least 4 years, or at least 5 years. For example, expression of the ASS1 can be sustained in the human subject for at least about 8 weeks, at least about 12 weeks, at least about 24 weeks, in certain embodiments, at least about 1 year, or at least about 2 years after treatment, and in some embodiments, at least 3 years, at least 4 years, or at least 5 years after treatment. Likewise, activity of the ASS1 can be sustained in the human subject for at least about 8 weeks, at least about 12 weeks, at least about 24 weeks, in certain embodiments for at least about 1 year, or at least about 2 years after treatment, and in some embodiments, at least 3 years, at least 4 years, or at least 5 years after treatment. In some methods, expression or activity of the ASS1 is maintained at a level higher than the expression or activity of the ASS1 prior to treatment (i.e., the subject's baseline). In some methods, expression or activity of the ASS1 is considered sustained if it is maintained at a therapeutically effective level of expression or activity. Relative durations, in other organisms, are understood based, e.g., on life span and developmental stages, are covered within the disclosure above. In some methods, expression or activity of the ASS1 is considered “sustained” if the expression or activity in a human at six months after administration, one year after administration, or two years after administration, the expression or activity is at least 50% of the expression or activity of the peak level of expression or activity measured for that subject. In certain embodiments, at six months, e.g., 24 weeks to 28 weeks, after administration the expression or activity is at least 50%, 55%, 60%, 65%, 70%, 75% or 80% of the expression or activity of the peak level of expression or activity measured for that subject. In certain embodiments, at one year, i.e., about 12 months, e.g., 11-13 months, after administration the expression or activity is at least 50%, 55%, 60%, 65%, 70%, 75% or 80% of the expression or activity of the peak level of expression or activity measured for that subject. In certain embodiments, at two years, i.e., about 24 months, e.g., 23-25 months, after administration the expression or activity is at least 50%, 55%, 60%, 65%, 70%, 75% or 80% of the expression or activity of the peak level of expression or activity measured for that subject. In certain embodiments, at six months after administration the expression or activity is at least 50%, preferably at least 60% of the expression or activity of the peak level of expression or activity measured for that subject. In certain embodiments, at one year after administration the expression or activity is at least 50%, preferably at least 60% of the expression or activity of the peak level of expression or activity measured for that subject. In certain embodiments, at two years after administration the expression or activity is at least 50%, preferably at least 60% of the expression or activity of the peak level of expression or activity measured for that subject. In preferred embodiments, the subject has routine monitoring of expression or activity levels of the ASS1, e.g., weekly, monthly, particularly early after administration, e.g., within the first six months. Periodic measurements may establish that the effect on expression or activity is sustained at, e.g., 6 months after administration, one year after administration, or two years after administration. In some methods in neonatal subjects, the expression of the ASS1 is sustained when the neonatal subject becomes an adult. In some methods, the expression of the ASS1 is sustained for the lifetime of the subject or neonatal subject.

In some methods, the expression or activity of the ASS1 is at least 50% of the expression or activity of the ASS1 at a peak level of expression measured for the human subject at 24 weeks after the administering. In some methods, the expression or activity of the ASS1 is at least 50% of the expression or activity of the ASS1 at a peak level of expression measured for the human subject at one year after the administering. In some methods, the expression or activity of the ASS1 is at least 60% of the expression or activity of the ASS1 at a peak level of expression measured for the human subject at 24 weeks after the administering. In some methods, expression or activity of the ASS1 is at least 50% of the expression or activity of the ASS1 at a peak level of expression measured for the human subject at two years after the administering. In some methods, the expression or activity of the ASS1 is at least 60% of the expression or activity of the ASS at a peak level of expression measured for the human subject at 2 years after the administering. In some methods, the expression or activity of the ASS is at least 60% of the expression or activity of the ASS1 at a peak level of expression measured for the human subject at 24 weeks after the administering.

In some methods, combination therapies are used comprising any of the compositions for expressing ASS1 disclosed herein together with an additional therapy suitable for treating citrullinemia type I or ASS1 deficiency. As one example, the methods described herein can be combined with the use of nitrogen scavenger medications (e.g., sodium phenylbutyrate, glycerol phenylbutyrate, or sodium benzoate). Additionally, or alternatively, treatment may further comprise dietary modification (e.g., protein restriction) and/or supplementation (e.g., arginine and/or carnitine supplementation). Additionally, or alternatively, the methods described herein can be combined with any therapeutically effective combination of one or more of the treatments of citrullinemia type I as set forth in Tables 13-15 below.

TABLE 13
Routine Daily Treatment in Individuals with Citrullinemia Type I

| Principle/Manifestation | Treatment | Considerations/Other |
|---|---|---|
| Protein restriction | In conjunction w/metabolic | Adequate protein intake can be based on |
| | nutritionist, lifelong protein | FAO/WHO/UNU 2007 “safe levels of |
| | restriction is required & varies | protein intake.” |
| | based on age of affected person. | If EAA supplements are needed, it is |
| | | reasonable to provide 20%-30% total |
| | | protein intake in this form. |
| Natural protein | The protein source for | Exclusive on-demand breastfeeding is |
| intake in infants | infants should be | possible but requires close analytic |
| | breastmilk or standard | monitoring & may require supplementation |
| | infant formula. | w/protein-free infant formula. |
| | Dietary therapy should | In bottle-fed infants, total daily protein |
| | be done in conjunction | amounts are divided evenly between daily |
| | w/metabolic | feeds, & can be supplemented w/protein- |
| | nutritionist. | free formula to round out caloric intake, |
| | | &/or to appetite, w/goal for total amount to |
| | | supply all required nutrients. |
| Nitrogen scavenger | Oral sodium phenylbutyrate | In persons weighing <20 kg: up to 250 |
| medications * | (BUPHENYL ®, | mg/kg |
| | AMMONAPS ®) | In persons weighing >20 kg: 5 g/m²/d, max |
| | | 12 g/d |
| | Glycerol phenylbutyrate | Initial dose for phenylbutyrate-naïve |
| | (RAVICTI ®) | persons: 4.5-11.2 mL/m²/d (5-12.4 g/m²/d), |
| | | max 17.5 mL/d |
| | | Dose for those transitioning from sodium |
| | | phenylbutyrate: daily dose of glycerol |
| | | phenylbutyrate (mL) = daily dose of |
| | | sodium phenylbutyrate (g) × 0.86 |
| | Sodium benzoate | Up to 250 mg/kg/d, max 12 g/d |
| Arginine | In persons weighing <20 | |
| supplementation | kg: 100-300 | |
| | mg/kg/d or 0.5-1.5 | |
| | mmol/kg/d | |
| | In persons weighing >20 | |
| | kg: 2.5-6 g/m²/d, | |
| | max 8 g/d | |
| Secondary carnitine | Initial oral dosage of 100 mg L- | Dose is adjusted on individual basis to maintain |
| deficiency | carnitine/kg/d divided into 3 or | plasma-free L-carnitine concentration w/in normal |
| | 4 doses is commonly used. | age-appropriate reference range. |
| Addressing ↑ energy/ | Fundoplication, gastrostomy, or | Adequate provision of info & education to parents, |
| caloric demands | jejunostomy to address feeding | affected persons, & caregivers |
| | issues | |

/d = per day;
EAA = essential amino acid;
FAO = Food and Agriculture Organization of the United States;
WHO = World Health Organization;
UNU = United Nations University
* Success of therapy is defined by a plasma ammonia concentration lower than 100 μmol/L and near-normal plasma glutamine concentration.

TABLE 14
Emergency Outpatient Treatment in Individuals with Citrullinemia Type I

| Manifestation | Treatment | Consideration/Other |
|---|---|---|
| Mildly increased | Carbohydrate supplementation | Trial of outpatient treatment at home for up to |
| catabolism ¹ | orally or via tube feed ² | 12 hrs; reassessment (~every 2 hrs) for clinical |
| | ↓ natural protein intake ³ | changes ⁵ |
| | ↑ carnitine supplementation ⁴ | |
| Fever | Administration of antipyretics | |
| | (acetaminophen, ibuprofen) if | |
| | temperature >38.5° C. | |
| Occasional | Antiemetics ⁶ | |
| vomiting | | |

¹ Fever <38.5° C. (101° F.); enteral or gastrostomy tube feeding tolerated without recurrent vomiting or diarrhea; absence of neurologic symptoms (altered consciousness, irritability, hypotonia, dystonia)
² Stringent guidelines to quantify carbohydrate/caloric requirements are available to guide nutritional arrangements in the outpatient setting, with some centers recommending frequent provision of carbohydrate-rich, protein-free beverages every two hours, with frequent reassessment.
³ Some centers advocate additional steps such as reducing natural protein intake to zero or to 50% of the normal prescribed regimen for short periods (<24 hours) in the outpatient setting during intercurrent illness.
⁴ Temporarily increasing L-carnitine doses (e.g., to 200 mg/kg/d in infants) may be considered.
⁵ Alterations in mentation/alertness, fever, and enteral feeding tolerance, with any new or evolving clinical features discussed with the designated center of expertise for inherited metabolic diseases
⁶ Some classes of antiemetics can be used safely on an occasional basis to temporarily improve enteral tolerance of food and beverages at home or during transfer to hospital.

TABLE 15
Acute Inpatient Treatment in Individuals with Citrullinemia Type I

| Manifestation | Treatment ¹ | Consideration/Other |
|---|---|---|
| Hyperammonemia | Withhold all protein intake for max 24-48 hrs. | This time frame allows for plasma ammonia |
| | | concentration to be ↓ & EAA deficiency that |
| | | would promote a catabolic state |
| | Pharmacologic nitrogen scavenger therapy ² | Priming/bolus infusion (given continuously |
| | | over 90 mins, dissolved in D10W [25-35 |
| | | mL/kg if <20 kg, in 1 L if >20 kg]): |
| | | Sodium benzoate: <20 kg: 250 |
| | | mg/kg; >20 kg: 5.5 g/m² |
| | | Sodium phenylacetate: <20 kg: 250 |
| | | mg/kg; >20 kg: 5.5 g/m² |
| | | 10% arginine HCl: <20 kg: 600 |
| | | mg/kg; >20 kg: 600 mg/kg |
| | | Sustaining/maintenance infusion (given |
| | | continuously over 24 hrs, dissolved in D10W |
| | | [25-35 mL/kg if <20 kg, in 1 L if >20 kg]): |
| | | Sodium benzoate: <20 kg: 250 |
| | | mg/kg; >20 kg: 5.5 g/m² |
| | | Sodium phenylacetate: <20 kg: 250 |
| | | mg/kg; >20 kg: 5.5 g/m² |
| | | 10% arginine HCl: <20 kg: 600 |
| | | mg/kg; >20 kg: 600 mg/kg |
| | Dialysis is most effective means of ↓ plasma | Failure to control ammonia |
| | ammonia rapidly. ³ | w/scavenger therapy requires |
| | | emergency use of dialysis. |
| | | Hemodialysis is preferred method of |
| | | dialysis & exceeds both peritoneal |
| | | dialysis & hemofiltration in rate of |
| | | ammonia clearance. |
| | | Continue scavenger therapy while |
| | | dialysis is being performed. |
| ↑ catabolism | Administer high-energy fluids & (if | Blood glucose, electrolyte |
| due to fever, | needed) insulin. ^{4, 5, 6} | concentrations, blood gases, plasma |
| perioperative/ | IV interlipids | amino acids, plasma carnitine |
| peri- | Consider L-carnitine | profiling, & urine pH/ketone |
| interventional | supplementation, esp if deficient. ⁷ | screening may all be useful in |
| fasting periods, | Address electrolytes & pH | guiding mgmt. |
| rptd vomiting/ | imbalances w/IV fluid mgmt. | Ongoing assessment of |
| diarrhea) | | hemodynamic status & for new |
| | | neurologic signs is critical. |
| | | Inadequate or delayed start of |
| | | emergency treatment → high risk of |
| | | neurologic injury w/consequent long- |
| | | term neurodisability. |
| | As soon as possible, osmolar load/ | To provide 0.25 g/kg/d of protein & |
| | hyperammonemia permitting, affected person | 50 kcal/kg/d, advancing (as plasma |
| | should receive TPN or enteral feeds. | ammonia concentration allows) to |
| | | 1.0-1.5 g/kg/d of protein & 100-120 |
| | | kcal/kg/d |
| | | Standard TPN solutions of dextrose, |
| | | aminosol, & intralipid are used. |
| Control of | Affected person should be maintained on dry | It is critical to monitor fluid balance, |
| intracranial | side of fluid balance: ~85 mL/kg of body | intake & output, & body weight. |
| pressure | weight per day in infants & appropriate | ↑ intracranial pressure is manifested |
| | corresponding fluid restriction in children & | by tension in fontanelle, acute |
| | adults. | enlargement of liver, edema, & |
| | | worsening neurologic signs incl |
| | | fisting, scissoring, ankle clonus, & |
| | | coma. |
| | | Cerebral edema & ischemia may be |
| | | documented by brain MRI. |

/d = per day;
EAA = essential amino acid;
IV = intravenous;
TPN = total parenteral nutrition
¹ Inpatient emergency treatment should: (1) take place at the closest medical facility, (2) be started without delay, and (3) be supervised by physicians and specialist dieticians at the responsible metabolic center, who should be contacted without delay.
² Repeat boluses are not recommended unless the individual is receiving dialysis.
³ Exchange transfusions have no place in hyperammonemic treatment.
⁴ IV glucose solutions should provide 12-15 g/kg/d glucose for infants and 10-12 g/kg/d for children age 12 months to 6 years.
⁵ In small infants, 40 kcal/100 mL given as D10W can be significant in averting catabolism
⁶ Use of insulin if hyperglycemia emerges; IV insulin given at a starting dose of 0.025 IU/kg/hour in the event of persistent hyperglycemia (>150-180 mg/dL in plasma, or glucosuria)
⁷ L-carnitine (with options to increase the dose) can be given intravenously, which enhances bioavailability.

In some methods, the method further comprises assessing preexisting anti-AAV (e.g., anti-AAV8 or anti-AAV5) immunity in a subject prior to administering any of the ASS1 nucleic acid constructs described herein. For example, such methods could comprise assessing immunogenicity using a total antibody (TAb) immune assay or a neutralizing antibody (NAb) assay. See, e.g., Manno et al. (2006) Nat. Med. 12(3):342-347, Kruzik et al. (2019) Mol. Ther. Methods Clin. Dev. 14:126-133, and Weber (2021) Front. Immunol. 12:658399, each of which is herein incorporated by reference in its entirety for all purposes. In some embodiments, TAb assays look for antibodies that bind to the AAV vector, whereas NAb assays assess whether the antibodies that are present stop the AAV vector from transducing target cells. With TAb assays, the drug product or an empty capsid can be used to capture the antibodies; NAb assays can require a reporter vector (e.g., a version of the AAV vector encoding luciferase).

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 16
Description of Sequences.

| SEQ ID NO | Type | Description |
|---|---|---|
| 1 | Protein | Human Argininosuccinate Synthase Protein NCBI Accession No. NP_000041.2 |
| 2 | DNA | Human ASS1 mRNA (cDNA) NCBI Accession No. NM_000050.4 |
| 3 | DNA | Human ASS1 CDS CCDS ID CCDS6933.1 |
| 4 | DNA | Human ASS1 Intron 2 |
| 5 | DNA | Mouse Ass1 Intron 1 |
| 6 | DNA | Guide RNA Target Sequence Plus PAM v1 |
| 7 | DNA | Guide RNA Target Sequence Plus PAM v2 |
| 8 | DNA | Guide RNA Target Sequence Plus PAM v3 |
| 9 | Protein | SpCas9 Protein V1 |
| 10 | DNA | SpCas9 DNA V1 |
| 11 | DNA | SpCas9 mRNA (cDNA) |
| 12 | Protein | SpCas9 Protein V2 |
| 13 | RNA | SpCas9 mRNA V2 |
| 14 | Protein | SV40 NLS v1 |
| 15 | Protein | SV40 NLS v2 |
| 16 | Protein | Nucleoplasmin NLS |
| 17 | RNA | crRNA Tail v1 |
| 18 | RNA | crRNA Tail v2 |
| 19 | RNA | TracrRNA v1 |
| 20 | RNA | TracrRNA v2 |
| 21 | RNA | TracrRNA v3 |
| 22 | RNA | gRNA Scaffold v1 |
| 23 | RNA | gRNA Scaffold v2 |
| 24 | RNA | gRNA Scaffold v3 |
| 25 | RNA | gRNA Scaffold v4 |
| 26 | RNA | gRNA Scaffold v5 |
| 27 | RNA | gRNA Scaffold v6 |
| 28 | RNA | gRNA Scaffold v7 |
| 29 | RNA | gRNA Scaffold v8 |
| 30 | RNA | Modified Generic sgRNA Using Scaffold v6 |
| 31-118 | RNA | Human ASS1 Intron 2 Guide Sequences |
| 119-206 | DNA | Human ASS1 Intron 2 Guide RNA Target Sequences |
| 207-212 | RNA | Mouse Ass1 Intron 1 Guide Sequences |
| 213-218 | DNA | Mouse Ass1 Intron 1 Guide RNA Target Sequences |
| 219-224 | RNA | Mouse Ass1 Intron 1 Full Guide Sequences - Scaffold v6 |
| 225 | DNA | Human ASS1 Exons 2-14 CDS with Stop Codon |
| 226 | DNA | Codon-Altered Human ASS1 Exons 2-14 CDS with Stop Codon |
| 227 | Protein | Human ASS1 Protein Encoded by Exons 2-14 |
| 228 | DNA | Human ASS1 Exons 3-14 CDS (native_v4) |
| 229 | DNA | Codon-Altered Human ASS1 Exons 3-14 CDS (co_v4) |
| 230 | Protein | Human ASS1 Protein Encoded by Exons 3-14 |
| 231 | DNA | Example 1 Episomal Construct ITR to ITR |
| 232 | DNA | Examples 2 and 4 Insertion Construct ITR to ITR |
| 233 | DNA | Example 5 Insertion Construct ITR to ITR (pAAV-ASS1-v4) |
| 234 | DNA | ApoE Enhancer |
| 235 | DNA | hGH polyA |
| 236 | DNA | bGH polyA v1 |
| 237 | DNA | bGH polyA v2 |
| 238 | DNA | bGH polyA v3 |
| 239 | DNA | SV40 polyA v1 |
| 240 | DNA | SV40 polyA v2 |
| 241 | DNA | ITR 145 |
| 242 | DNA | ITR 141 |
| 243 | DNA | ITR 130 |
| 244 | DNA | hAAT promoter |
| 245 | DNA | HiBiT Tag CDS |
| 246 | DNA | Codon-Altered HiBiT Tag CDS |
| 247 | Protein | HiBIT Tag |
| 248 | DNA | Mouse Alb exon 2 Splice Acceptor |
| 249 | RNA | Cas9 mRNA |
| 250 | RNA | Cas9 mRNA CDS |
| 251 | DNA | Cas9 CDS |
| 252-339 | RNA | Human ASS1 Intron 2 Full Guide Sequences - Scaffold v6 |
| 340-427 | RNA | Human ASS1 Intron 2 Full Guide Sequences - Scaffold v9 |
| 428 | RNA | gRNA Scaffold v9 |
| 429 | RNA | Modified Generic sgRNA Using Scaffold v9 |
| 430-517 | RNA | Human ASS1 Intron 1 Guide Sequences |
| 518-605 | DNA | Human ASS1 Intron 1 Guide RNA Target Sequences |
| 606-693 | RNA | Human ASS1 Intron 1 Full Guide Sequences - Scaffold v9 |
| 694 | DNA | Example 6 Insertion Construct ITR to ITR |
| 695 | DNA | Human ASS1 Exons 2-14 CDS |
| 696 | DNA | Codon-Altered Human ASS1 Exons 2-14 CDS |
| 697-702 | RNA | Mouse Ass1 Intron 1 Full Guide Sequences - Scaffold v9 |
| 703 | DNA | Insertion Construct 55 [ASS1_cpg5v2b] - ITR to ITR |
| 704 | DNA | Insertion Construct 55 [ASS1_cpg5v2b] - Splice Acceptor to Splice Acceptor |
| 705 | DNA | ASS1 Partial cDNA CDS1 |
| 706 | DNA | ASS1 Partial cDNA CDS2 |
| 707 | DNA | Insertion Construct 56 [ASS1_cpg9v8b] full sequence from ITR to ITR |
| 708 | DNA | Insertion Construct 56 [ASS1_cpg9v8b] full sequence from splice acceptor to splice |
| | | acceptor |
| 709 | DNA | ASS1 partial cDNA CDS3 |
| 710 | DNA | ASS1 partial cDNA CDS4 |
| 711 | DNA | Insertion Construct 57 [ASS1_cpg15b] full sequence from ITR to ITR |
| 712 | DNA | Insertion Construct 57 [ASS1_cpg15b] full sequence from splice acceptor to splice |
| | | acceptor |
| 713 | DNA | ASS1 partial cDNA CDS5 |
| 714 | RNA | G040925 Guide Sequence |
| 715 | DNA | G040925 Target Sequence |
| 716 | RNA | G040925 sgRNA Sequence with Scaffold v9 |

### EXAMPLES

### Example 1. ASS1 Episomal AAV Efficacy Study in Adolescent P28 Fold Citrullinemia Mice

Chow fed 4-week old Ass1^{fold/fold} citrullinemia mice (also referred to as fold citrullinemia mice) were dosed intravenously with recombinant AAV8-AAT-hASS1 episomal vector at 1E+13 vg/kg and 3E+13 vg/kg, whereas control Ass1^{fold/fold} mice were dosed with a GFP-encoding recombinant AAV8 episomal vector at 1E+13 vg/kg. The AAV8-AAT-hASS1 construct comprises from 5′ to 3′: an ApoE enhancer (SEQ ID NO: 234), a hAAT promoter (SEQ ID NO: 244), an hASS1 coding sequence (SEQ ID NO: 3, encoding SEQ ID NO: 1), and an hGH polyA sequence (SEQ ID NO: 235). The complete sequence for the construct, include the ITRs, is set forth in SEQ ID NO: 231. Untreated wild type and heterozygous Ass1^{fold/+} mice served as healthy benchmark controls. See Table 17. Mice were bled at 1, 2, 4, 6, 8, 12, 16, and 20 week intervals post-treatment to assess plasma ammonia and citrulline levels. See FIG. 1. Mice were euthanized at 20 weeks post-treatment, and liver tissue was collected and processed for total RNA extraction and FFPE histology. ASS1 and GFP mRNA expression was measured from total RNA using gene specific TaqMan probes. ASS1 mRNA histological expression was determined using a human-specific BaseScope in situ hybridization (ISH) probe. ASS1 and GFP protein expression was determined in liver sections using immunohistochemistry (IHC).

TABLE 17
ASS1 episomal AAV efficacy study in adolescent P28 fold citrullinemia mice.

| Group | Genotype | Treatment | Dose | Route | Age | Sex | n |
|---|---|---|---|---|---|---|---|
| 1 | Ass1-fold/fold | Untreated | n/a | n/a | P28 | Mixed | 6 |
| 2 | Ass1-fold/fold | AAV8-CAG-eGFP | 1e13 vg/kg | RO | P28 | Mixed | 5 |
| 3 | Ass1-fold/fold | AAV8-hAAT-hASS1 | 1e13 vg/kg | RO | P28 | Mixed | 5 |
| 4 | Ass1-fold/fold | AAV8-hAAT-hASS1 | 3e13 vg/kg | RO | P28 | Mixed | 5 |
| 5 | WT & het | Untreated | n/a | n/a | P28 | Mixed | 5 |
| | combined | | | | | | |

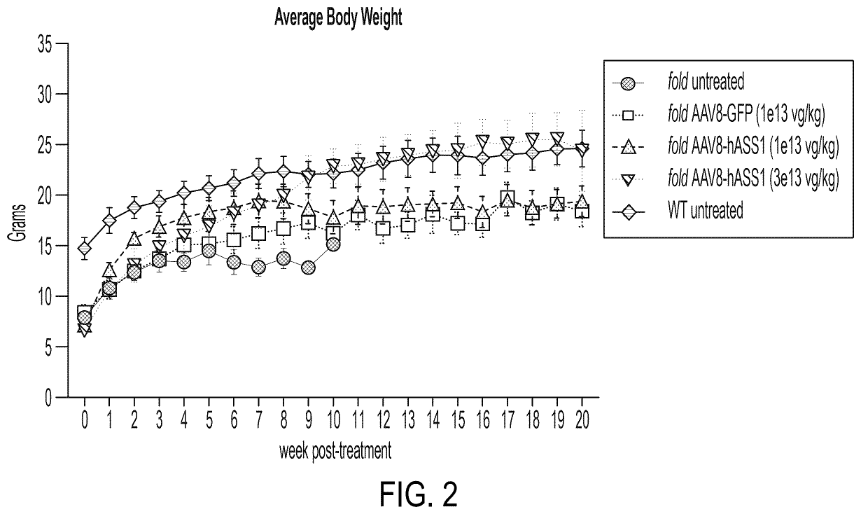

As shown in FIG. 2, 3E13 vg/kg hASS1 episome treatment at P28 rescued stunted growth in fold/fold mice. The fold/fold mice weighed less than half of wild type littermates at P28 (6.6 g vs. 14.7 g), and the 3E13 vg/kg treatment group caught up to the untreated wild type group by 9 weeks post-treatment.

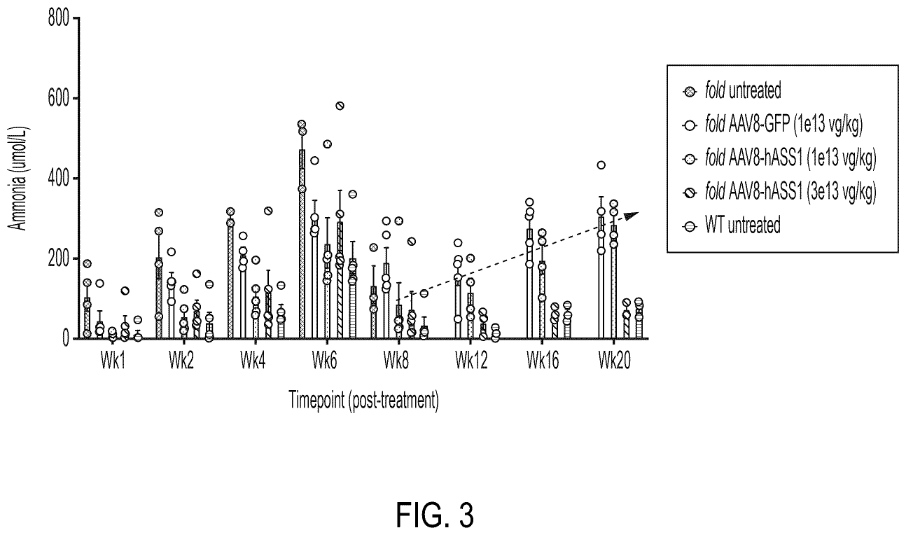

As shown in FIG. 3, episomal hASS1 treatment (3e13 vg/kg) rescues the hyperammonemia phenotype in fold/fold mice, but there were signs of limited durability with episomal treatment. Untreated fold/fold mice develop worsening hyperammonemia leading to death at 10-12 weeks of age. Both doses of hASS1 episome showed comparable efficacy at early timepoints. However, a reduction of efficacy of AAV8-AAT-hASS1 episomal vector was observed at dose 1E+13 vg/kg in Ass1^{fold/fold} mice at week 12 post-treatment with slow but robust elevation of plasma ammonia in the following weeks. Dosing of Ass1^{fold/fold} mice with 3E+13 vg/kg of AAV8-AAT-hASS1 episomal vector showed fully corrected plasma ammonia levels compatible with the wild type mouse group through the end of experiment.

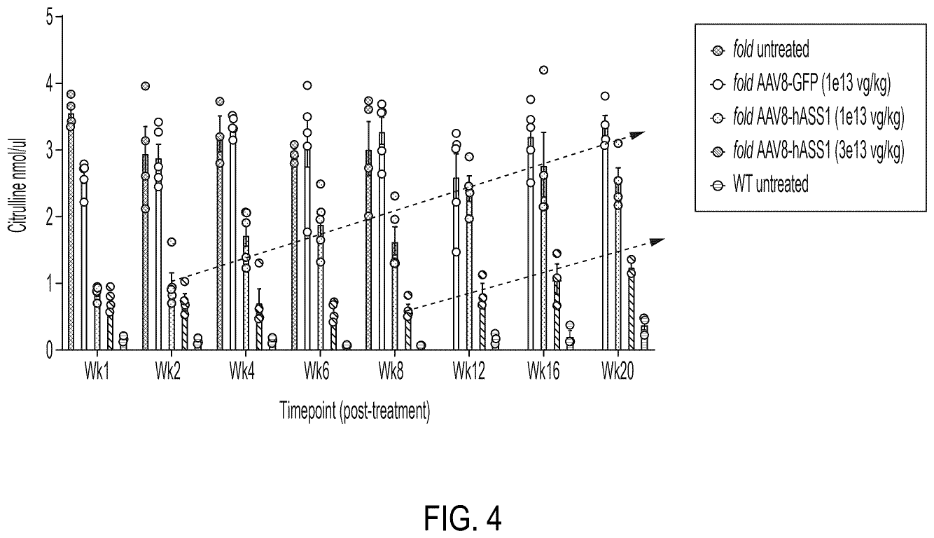

As shown in FIG. 4, the citrulline response mirrored the ammonia response, with signs of limited durability with episomal treatment. Citrulline levels were elevated more than 20-fold in fold/fold mice. Citrulline rapidly decreased by 76% and 79% after only 1 week of ASS1 episome treatment. However, a reduction of efficacy of AAV8-AAT-hASS1 episomal vector was observed at dose 1E+13 vg/kg at week 4 and at dose 3E+13 vg/kg in Ass1^{fold/fold} mice at week 12, with the slow but robust elevation of plasma citrulline in following weeks.

### Example 2. ASS1 Gene Insertion Efficacy Study in Adolescent P28 Fold Citrullinemia Mice

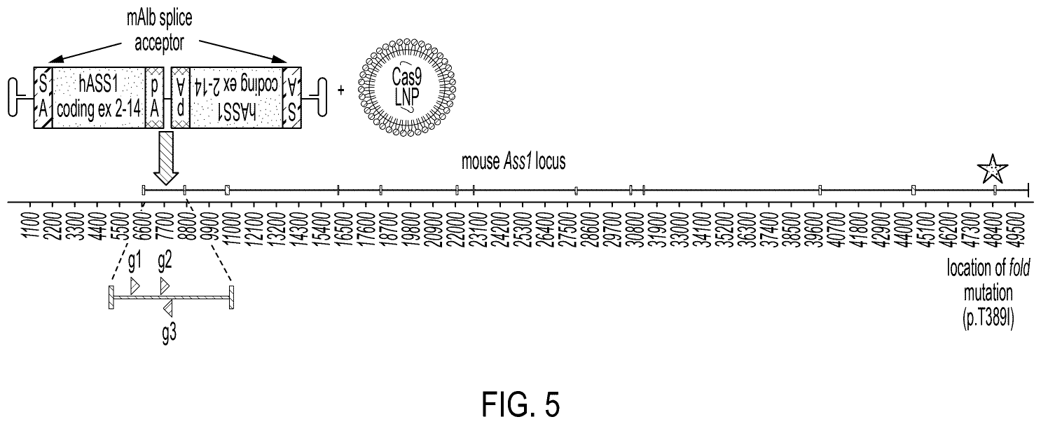

To model a therapeutic approach in humans, a mouse Ass1 native locus gene insertion strategy in Ass1^{fold/fold} citrullinemia mice was used as shown in FIG. 5. The approach is based on the CRISPR/Cas9 gene editing technology contained in a lipid nanoparticle (LNP) delivery system, associated with a bidirectional ASS1 DNA gene insertion template contained in a recombinant adeno-associated virus serotype 8 (rAAV8). The CRISPR/Cas9 component was designed to target and cut the double stranded DNA at the Ass1 gene locus in hepatocytes, allowing for the ASS1 DNA template, delivered by AAV vectors, to be inserted in the genome at the endogenous Ass1 gene locus encoding the defective genomic Ass1. Transgene insertion provides a functional ASS1 gene. In this experiment, Cas9 mRNA and a gRNA targeting intron 1 of the mouse Ass1 locus were delivered together with a bidirectional insertion template containing hASS1 coding sequence to fold mice. The transgene is inserted into intron 1, between coding exons 1 and 2. The rAAV-delivered ASS1 DNA cassette is promoterless and instead harnesses transcription of the insertion locus (the endogenous Ass1 locus) to drive expression of the therapeutic transgene encoding the ASS1 protein. The rAAV8-delivered DNA insertion template does not have any homology to the nucleotides flanking the Ass1 sgRNA target site and is inserted via homology-independent mechanisms. Therefore, the insertion cassette was designed to be bidirectional and encodes a copy of wild type ASS1 protein in both the sense and antisense orientation, such that either insertion orientation can support ASS1 protein expression. However, a unidirectional insertion cassette can also be used. A splice acceptor site is encoded upstream of each ASS1 transgene, and a polyadenylation sequence is encoded downstream of each copy of ASS1. The endogenous Ass1 exon 1 splice donor is thus joined with the splice acceptor encoded in the ASS1 DNA template.

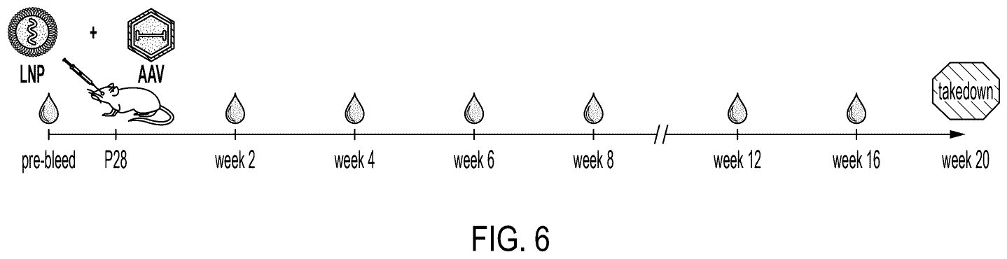

Specifically, chow fed 4-week old Ass1^{fold/fold} citrullinemia mice were dosed intravenously with recombinant AAV8-ASS1_v2b (contains human ASS1 gene exon 2-14, no promoter) at dose 3E+12 vg/kg or 3E+13 vg/kg plus 1 mg/kg lipid nanoparticles (LNP) with Cas9 mRNA/gRNA, whereas control Ass1^{fold/fold} mice were dosed with a recombinant AAV8-ASS1_v2b at dose 3E+13 vg/kg only (no LNP). See Table 18. Untreated wild type and Ass1^{fold/fold} mice served as benchmark controls. The bidirectional ASS1 DNA gene insertion template (AAV8-ASS1_v2b_ins) comprises from 5′ to 3′: Alb splice acceptor (SEQ ID NO: 248), native human ASS1 exons 2-14 coding sequence (SEQ ID NO: 225, encoding SEQ ID NO: 227), bGH polyA (SEQ ID NO: 236), reverse complement of SV40 polyA (reverse complement of SEQ ID NO: 239), reverse complement of codon altered human ASS1 exons 2-14 coding sequence (reverse complement of SEQ ID NO: 226, encoding SEQ ID NO: 227), and reverse complement of Alb splice acceptor (reverse complement of SEQ ID NO: 248). The complete AAV8-ASS1_v2b_ins construct including ITRs is set forth in SEQ ID NO: 232. The mouse Ass1 guide RNA that was used is set forth in SEQ ID NO: 699 (DNA-targeting segment set forth in SEQ ID NO: 209, and guide RNA target sequence set forth in SEQ ID NO: 215). Mice were bled the day before treatment and at 2, 4, 6, 8, 12, 16, and 20 week intervals post-treatment to assess plasma ammonia and citrulline levels as well body weight changes. See FIG. 6. Mice were euthanized at 20 weeks post-treatment, and liver tissue was collected and processed for total RNA extraction and FFPE histology. ASS1 mRNA expression was measured from total RNA using gene-specific TaqMan probes. ASS1 mRNA histological expression was determined using a human-specific BaseScope in situ hybridization (ISH) probe. ASS1 protein expression was determined in liver sections using immunohistochemistry (IHC). Liver DNA was extracted to measure CRISPR/Cas9-mediated deletions/insertions (“indels”, shown as %), measured by droplet digital PCR (ddPCR).

TABLE 18
ASS1 gene insertion efficacy study in adolescent P28 fold citrullinemia mice.

| Group | Genotype | Treatment | Dose | Route | Age | Sex | n |
|---|---|---|---|---|---|---|---|
| 1 | Ass1-fold/fold | Untreated | n/a | n/a | P28 | Mixed | 3 |
| 2 | Ass1-fold/fold | AAV8-ASS1_v2b_ins | 3e13 vg/kg AAV | RO | P28 | Mixed | 3 |
| | | (no LNP) | | | | | |
| 3 | Ass1-fold/fold | AAV8-ASS1_v2b_ins + | 3e12 vg/kg AAV + | RO | P28 | Mixed | 4 |
| | | Ass1 LNP g3 | 1 mg/kg LNP | | | | |
| 4 | Ass1-fold/fold | AAV8-ASS1_v2b_ins + | 3e13 vg/kg AAV + | RO | P28 | Mixed | 5 |
| | | Ass1 LNP g3 | 1 mg/kg LNP | | | | |
| 5 | WT | Untreated | n/a | n/a | P28 | Mixed | 5 |

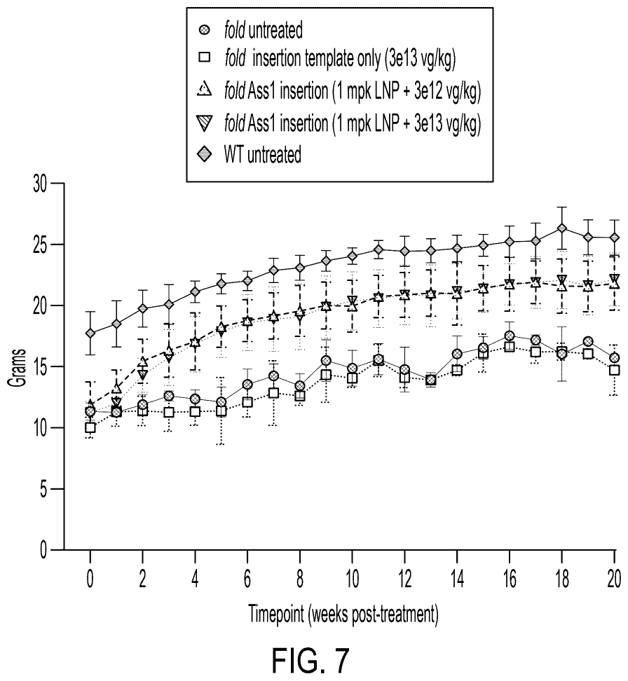

As shown in FIG. 7, both mice groups that received co-administration of AAV8-ASS1_v2b plus LNP achieved partial rescue of the initial low body weights observed in Ass1^{fold/fold} mice, but rescued body weights did not reach wild type mice levels. A control group dosed only with the insertion template did not show any body weight improvement over the untreated Ass1^{fold/fold} mice control group. A potential explanation for the partial rescue is that Ass1^{fold/fold} mice dosed at 28 days old already have a long term effect of untreated hyperammonemia with changes in the brain and other parts of the body, in contrast to mice dosed at 7 days old (see Example 4).

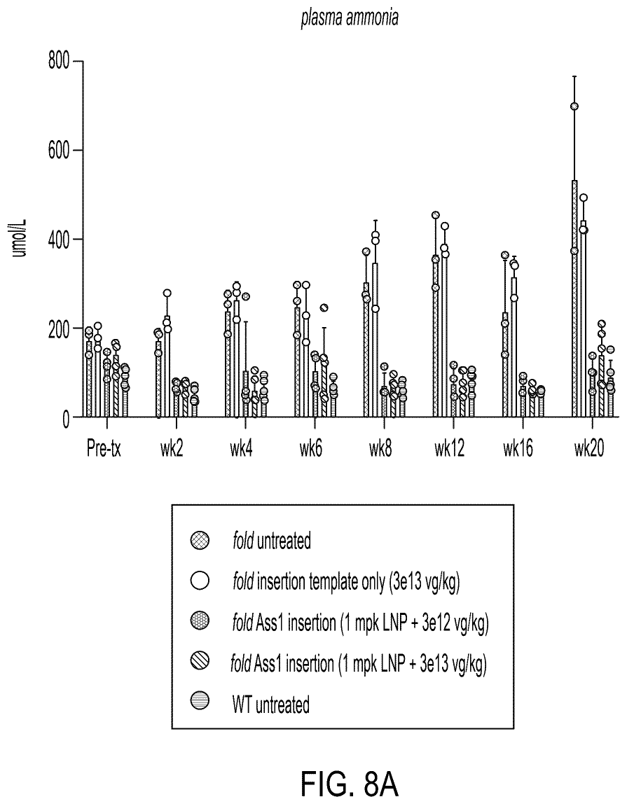

As shown in FIGS. 8A-8B, serum ammonia levels in untreated and insertion template-only treated Ass1^{fold/fold} mice gradually increased through the end of the experiment at week 20. In both Ass1^{fold/fold} mice groups that received co-administration of AAV8-ASS1_v2b plus LNP, ammonia levels stayed at levels of wild type mice through the end of experiment (full rescue). After treatment, serum citrulline dropped in 2 weeks and stayed stable through the end of experiment in both co-administered Ass1^{fold/fold} mice groups (3E+12 vg/kg or 3E+13 vg/kg plus 1 mg/kg lipid LNP with Cas9/gRNA), whereas plasma citrulline levels continued to increase in control Ass1^{fold/fold} mice treated only with AAV insertion template, comparable with untreated fold mice. The difference between full correction of plasma ammonia but not citrulline could be explained by fact that, in wild type mouse, ASS1 is also expressed in kidneys, and coadministration of AAV8-ASS1_v2b plus LNP in Ass1^{fold/fold} mice only compensates for ASS1 deficiency in liver. In those mice, some of the kidney-produced citrulline must be transported to liver with blood for future metabolism. Such discrepancies between serum ammonia and citrulline are also observed in citrullinemia type I patients after orthotopic liver transplantation, but this finding does not affect patient general condition.

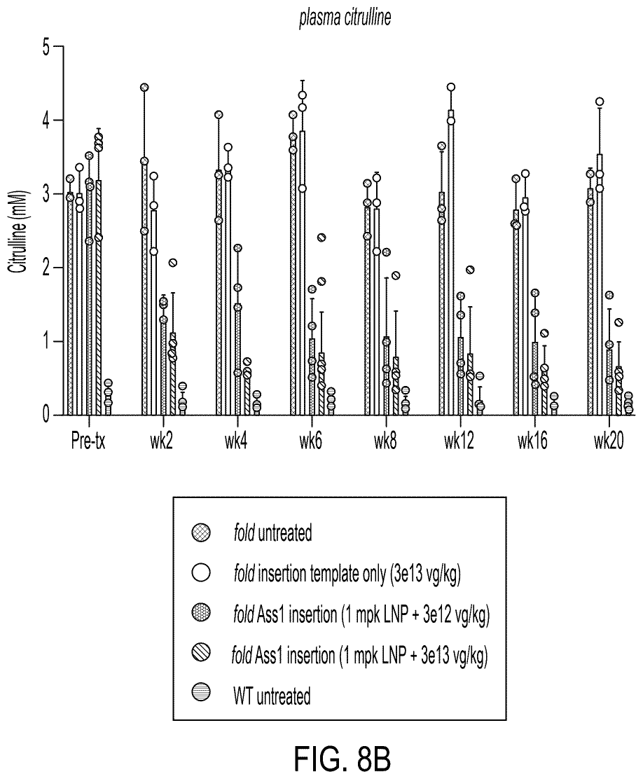

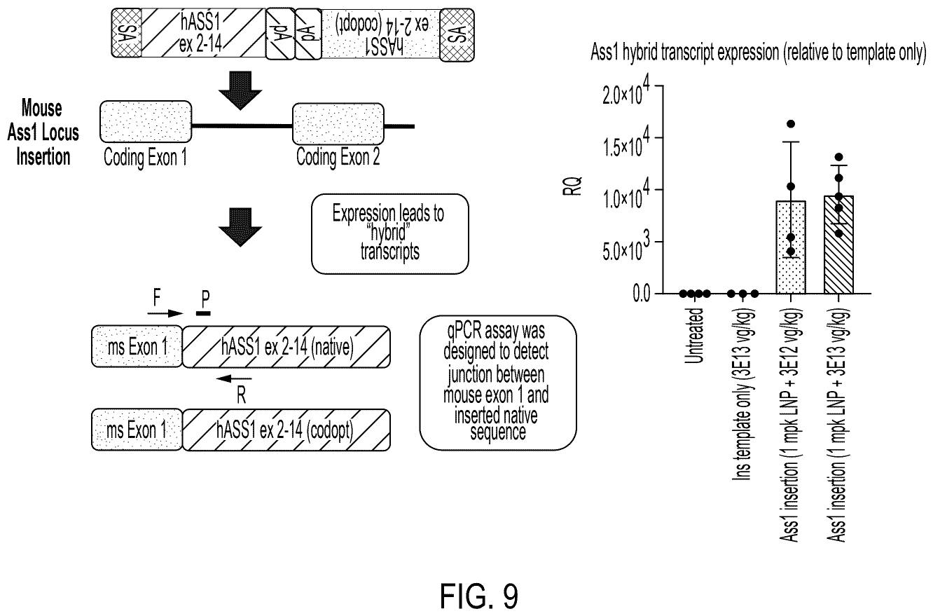

As shown in FIG. 9, specific RT-qPCR (Taqman) probes were designed to detect mouse exon 1 (F) and inserted human ASS1 exon 2 (R) to detect hybrid (mouse/human) ASS1 expression. Measurement of hybrid ASS1 expression in mouse liver showed no expression in untreated and AAV template treated only Ass1^{fold/fold} mice groups. High and relatively similar expression of hybrid ASS1 was detected in both co-treated fold mice groups. Similar results were observed with droplet digital PCR on liver genomic DNA preparations designed to quantify the percentage of mouse Ass1 alleles containing a gene insertion at mouse Ass1 intron 1. See FIG. 10.

Using an anti-ASS1 antibody that detects both human and mouse ASS1 proteins for liver immunohistochemistry (IHC) of mouse livers, we observed mild ASS1 staining in control fold mice (no treatment or AAV insertion template only), but the signal was weaker than in untreated wild type mice (data not shown). Both Ass1^{fold/fold} mice groups that received co-administration of AAV8-ASS1_v2b (3E+12 vg/kg or 3E+13 vg/kg) plus 1 mg/kg lipid LNP with Cas9/gRNA showed more intense, patchy expression of human ASS1 in livers (data not shown).

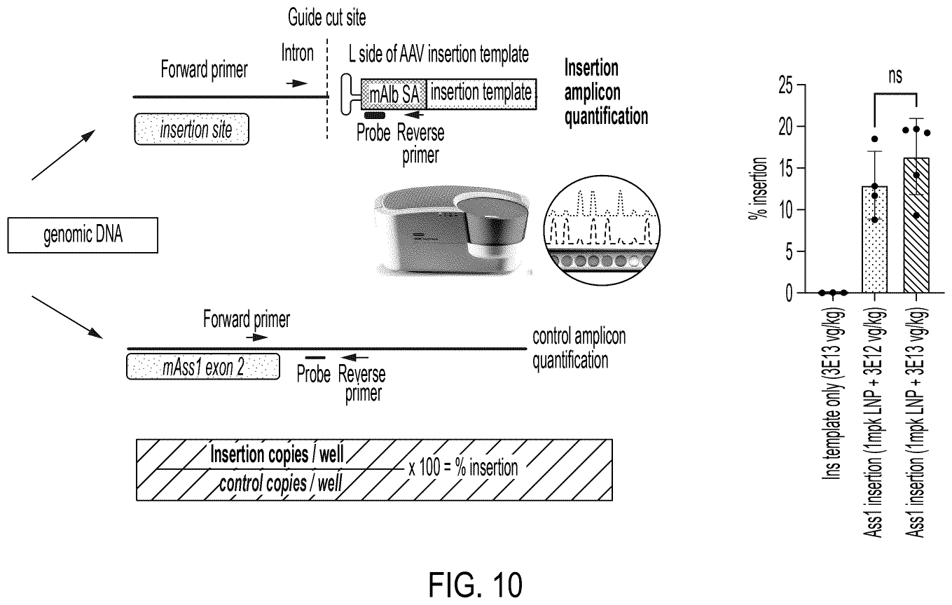

As shown in FIG. 11, hybrid (mouse/human fusion) ASS1 mRNA in situ hybridization (ISH) staining (BaseScope) showed that wild type and both fold mice controls (no treatment or AAV insertion template only) did not show ASS1 expression. Both Ass1^{fold/fold} mice groups that received co-administration of AAV8-ASS1_v2b (3E+12 vg/kg or 3E+13 vg/kg) plus 1 mg/kg lipid LNP) showed expression of ASS1 in livers (around 40% of hepatocytes contained ASS1 mRNA localized in cytosol).

### Example 3. ASS1 Episomal AAV Efficacy Study in P7 Fold Citrullinemia Mice

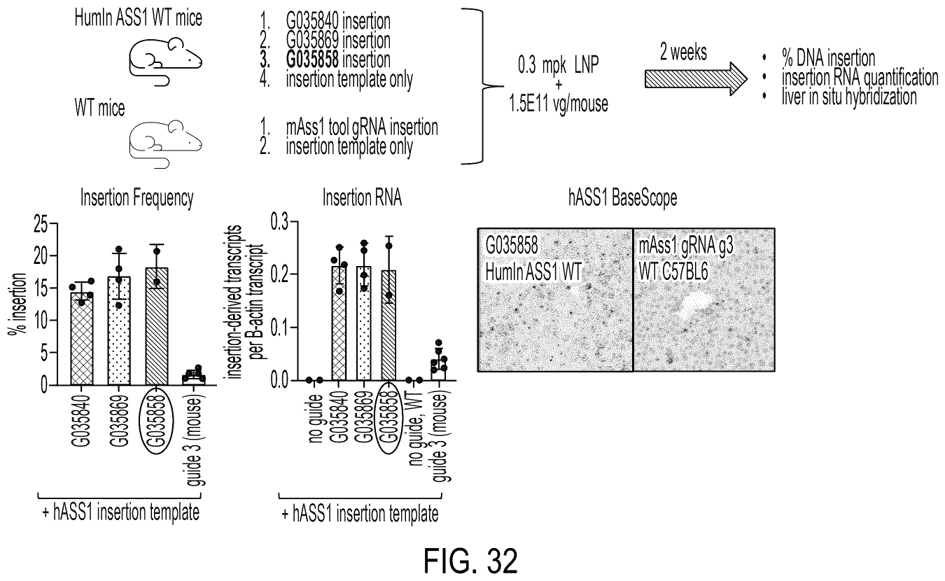

To evaluate the therapeutic potential of AAV-delivered episomal ASS1 transgenes in alleviating disease biomarkers, 1-week old Ass1^{fold/fold} citrullinemia mice were administered AAV8 viral vectors containing promoter-driven hASS1-expressing transgene (hAAT-hASS1) or control eGFP transgene (CAG-eGFP), at a dose of 3E10 vector genomes per mouse intravenously via retro-orbital injection and maintained on normal chow diet. Blood was collected bi-weekly for plasma ammonia and citrulline measurement. Body weights were measured weekly. The observational period was 20 weeks. Episomal AAV8-hAAT-hASS1 treatment elicited minimal improvement in body weight, and plasma ammonia and citrulline levels, indicating minimal therapeutic efficacy in citrullinemia mice when administered early in life. See FIG. 31. This likely results from episome dilution and transgene loss due to ongoing hepatocyte proliferation during the neonatal period, and highlights the need for more durable hepatic ASS1 transgene maintenance and expression.

### Example 4. ASS1 Gene Insertion Efficacy Study in P7 Fold Citrullinemia Mice

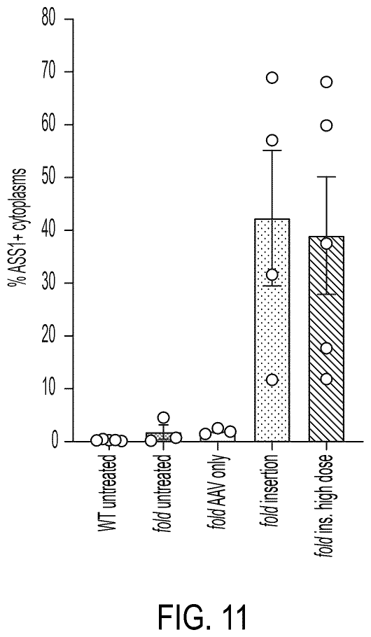

Using the same approach, insertion template, and guide RNAs as in Example 2, chow-fed, 7-day old Ass1^{fold/fold} citrullinemia mice were dosed intravenously with recombinant AAV8-ASS1_v2b (contains human ASS1 gene exon 2-14, no promoter) at dose 3E+12 vg/kg or 3E+13 vg/kg plus 1 mg/kg LNP with Cas9/gRNA, whereas control Ass1^{fold/fold} mice were dosed with a recombinant AAV8-ASS1_v2b at 3E+13 vg/kg only but no LNP. See Table 19. Untreated wild type and Ass1^{fold/fold} mice served as benchmark controls. Mice were bled at 2, 4, 6, 8, 12, 16, and 20 week intervals post-treatment to assess plasma ammonia and citrulline levels. See FIG. 12. Mice were euthanized at 20 weeks post-treatment, and liver tissue was collected and processed for total RNA extraction and FFPE histology. ASS1 mRNA expression was measured from total RNA using gene specific TaqMan probes. ASS1 mRNA histological expression was determined using a human-specific BaseScope in situ hybridization (ISH) probe. ASS1 protein expression was determined in liver sections using immunohistochemistry (IHC).

TABLE 19
ASS1 gene insertion efficacy study in P7 fold citrullinemia mice.

| Group | Genotype | Treatment | Dose | Route | Age | Sex | n |
|---|---|---|---|---|---|---|---|
| 1 | Ass1-fold/fold | Untreated | n/a | n/a | P7 | Mixed | 5 |
| 3 | Ass1-fold/fold | AAV8-ASS1_v2b_ins | 3e10 vg AAV | RO | P7 | Mixed | 4 |
| | | (no LNP) | | | | | |
| | | (template only) | | | | | |
| 4 | Ass1-fold/fold | AAV8-ASS1_v2b_ins + | 3e10 vg AAV + 2 | RO | P7 | Mixed | 4 |
| | | Ass1 LNP g3 | mg/kg LNP | | | | |
| | | (high dose insertion) | | | | | |
| 5 | Ass1-fold/fold | AAV8-ASS1_v2b_ins + | 3e10 vg AAV + 1 | RO | P7 | Mixed | 4 |
| | | Ass1 LNP g3 | mg/kg LNP | | | | |
| | | (medium dose insertion) | | | | | |
| 7 | WT | Untreated | n/a | n/a | P7 | Mixed | 5 |

3e10 vg/mouse = ~1e13 mg/kg

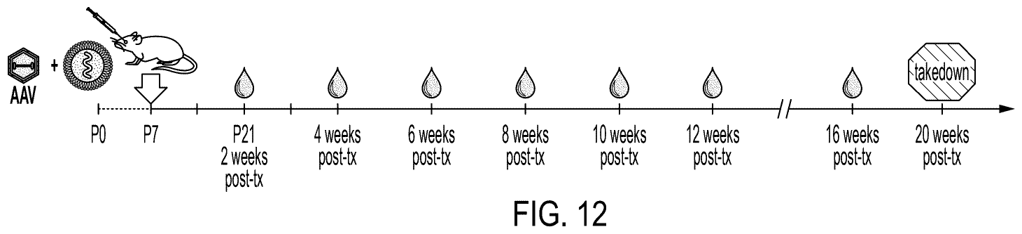

As shown in FIG. 13, both Ass1^{fold/fold} mice groups that received coadministration of recombinant AAV8-ASS1_v2b plus LNP showed a complete rescue Ass1^{fold/fold} mouse delayed body weight gain, with values reaching wild type mice levels. A control group of Ass1^{fold/fold} mice dosed only with the insertion template did not show any improvement over the untreated control fold mice group.

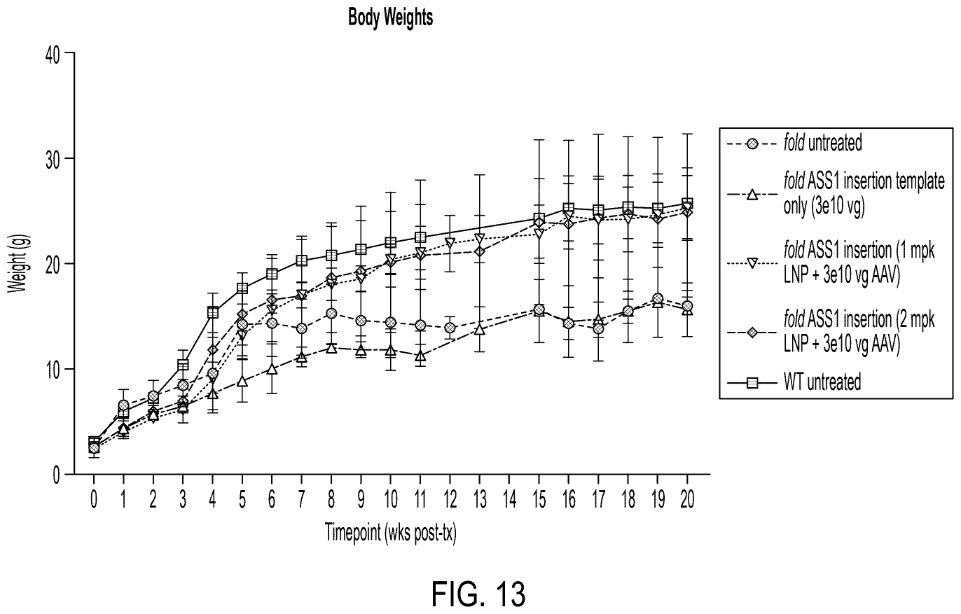

As shown in FIGS. 14A-14B, both control fold mice groups (with no treatment or insert delivery only) showed gradual plasma ammonia increase over time. Ass1^{fold/fold} mice after coadministration of AAV8-ASS1_v2b plus LNP showed low plasma ammonia comparable with wild type littermates for all experimental time points. Similar to ammonia, plasma citrulline stayed high in both untreated and insert-only treated fold mice groups, while coadministration of AAV8-ASS1_v2b plus LNP in Ass1^{fold/fold} mouse resulted in steady reduction of citrulline, but values did not reach untreated wild type levels. As mentioned above, ammonia is a more reliable marker for Ass1 gene correction in liver, as citrulline produced in kidney causes serum citrulline elevation even after liver ASS1 deficit correction.

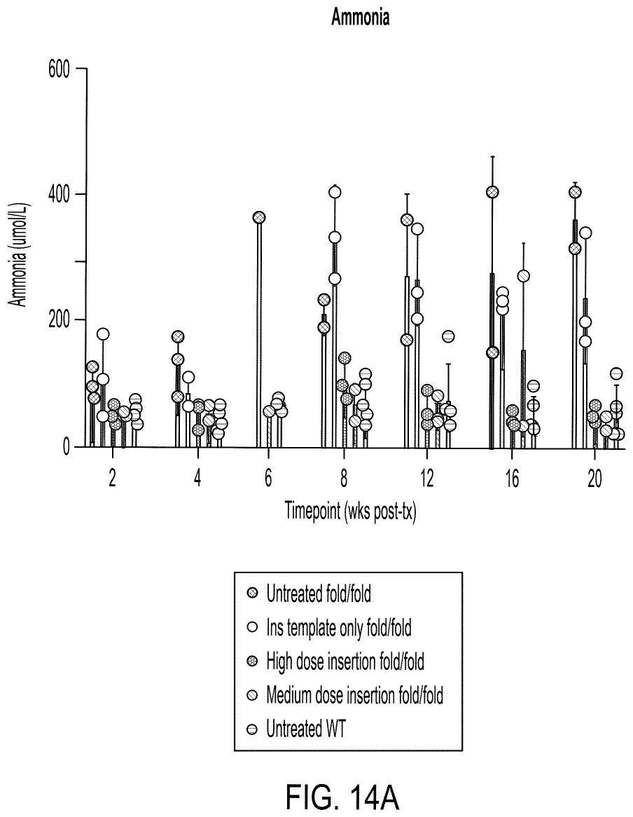

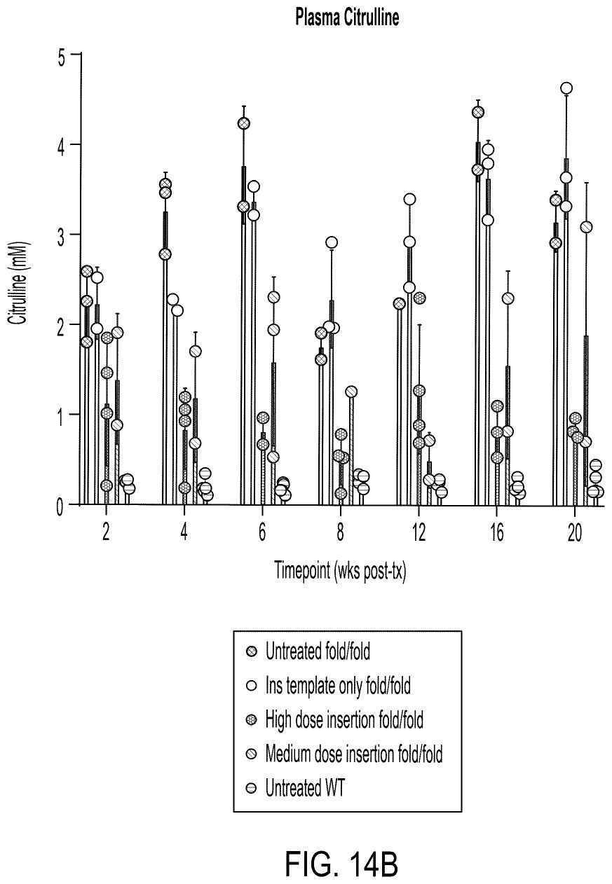

As shown in FIG. 15, hybrid (mouse/human) ASS1 mRNA expression was observed in both groups of Ass1^{fold/fold} mouse after coadministration of AAV8-ASS1_v2b plus LNP, whereas all other control groups showed no expression of ASS1 mRNA in liver. Similar results were observed with droplet digital PCR. See FIG. 16. Co-treatment (ASS1 AAV8 plus LNP) of fold mice resulted in 10-15% hASS1 insertion in mouse livers.

Using an anti-ASS1 antibody that detects both human and mouse ASS1 proteins for liver immune histochemistry (IHC) of mouse livers, we observed that both Ass1^{fold/fold} mouse groups after co-administration of AAV8-ASS1_v2b plus LNP, but not AAV8-ASS1_v2b insert only, demonstrated successful insertion and expression of hASS1 in liver (data not shown). Wild type mouse ASS1 protein stains more uniformly in the cytoplasm, whereas the fold variant appears more punctate (data not shown).

ASS1 mRNA in situ hybridization (ISH) staining (BaseScope) was performed on fixed livers with RNA probe for detection of human ASS1. The data showed that both Ass1^{fold/fold} mouse groups that received coadministration of AAV8-ASS1_v2b and LNP, but not AAV8-ASS1_v2b AAV8 only, demonstrated successful insertion and expression of hASS1 in liver (data not shown).

The forgoing examples demonstrated that ASS1 gene insertion may provide an effective and durable treatment that can be administered early in life to promptly halt disease progression and onset of irreversible manifestations of citrullinemia. In the approach disclosed above, lipid nanoparticles were used to transiently deliver Cas9 mRNA and an Ass1-directed guide RNA, co-administered with AAV containing a promoter-less ASS1 partial cDNA for insertion into the native Ass1 chromosomal gene locus. Proof-of-concept studies performed in the lethal Ass1^{fold/fold} citrullinemia mouse model, in both young adult (P28; Example 2) and newborn (P7, Example 4) Ass1^{fold/fold} mice, demonstrated that Ass1 native locus gene insertion was highly effective and durable at preventing elevation of blood ammonia and citrulline. Histological and molecular analyses demonstrated efficient targeting and correction of the Ass1 locus in a significant fraction of hepatocytes. Comparable efficacy was observed not only in young adult (P28) Ass1^{fold/fold} mice, but also in newborn (P7) mice where significant hepatocyte proliferation associated with normal liver growth remains. This was in stark contrast to ASS1 gene therapy delivered via conventional episomal AAV. We observed minimal correction of ammonia and citrulline levels when administering an ASS1-encoding AAV episome to P7 Ass1^{fold/fold} neonates (data not shown), and only transient efficacy in young adult P28 fold mice (Example 1). Noticeable differences were further observed in zonal hepatocyte correction between Ass1 native locus gene insertion and AAV episome treatment approaches. Ass1 native locus gene insertion led to preferential ASS1 restoration in peri-portal hepatocytes with high urea cycle activity, whereas AAV episome approach surprisingly led to ASS1 restoration preferentially in low urea cycle activity pericentral (i.e., centrilobular) hepatocytes (data not shown). ASS1 expression and urea cycle activity is naturally lowest around central veins (i.e., centrilobular/pericentral region) and highest around portal veins (i.e., periportal region). With native locus insertion, expression of the inserted hASS1 was driven by the endogenous Ass1 locus and promoter and was localized to urea-cycle active periportal and midzonal hepatocytes. In contrast, hASS1 expression via AAV episome led to low expression and localization of hASS1 in centrilobular hepatocytes, which are not typically urea-cycle-active and presents challenges for episome-based therapies. This suggests possibly enhanced turnover of periportal hepatocytes in citrullinemic Ass1^{fold/fold} mice, which would be a major obstacle for episome-based therapies.

### Example 5. Human ASS1 Gene Insertion Guide RNA Screen (Intron 2)

The human ASS1 locus, specifically intron 2 between coding exons 2 and 3, was evaluated for the presence of the specific CRISPR/Cas9 protospacer adjacent motifs (PAM) sites to generate a list of target sites in the first intron of the locus where a Cas9-induced DNA cut could occur. The sites were cross-referenced against a single nucleotide polymorphism (SNP) analysis to ensure selected sites did not have common SNPs that might impact gRNA efficiency. A list of sgRNAs was then generated, and guide RNAs were synthesized and formulated into LNPs with Cas9 mRNA for evaluation in vitro and in vivo.

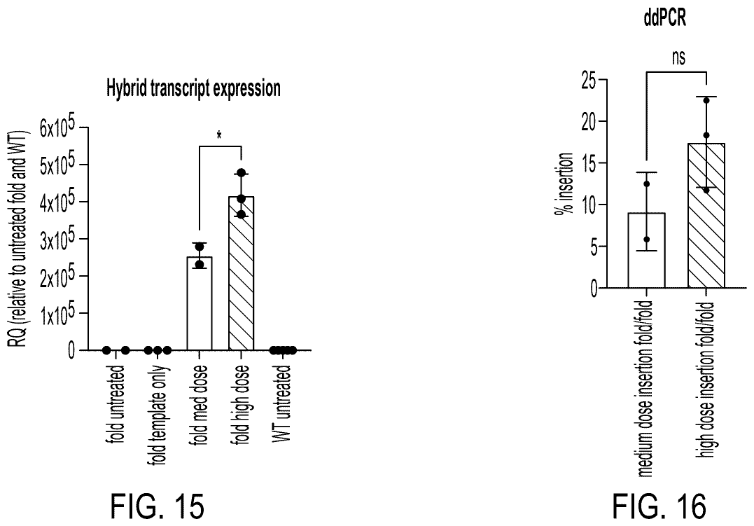

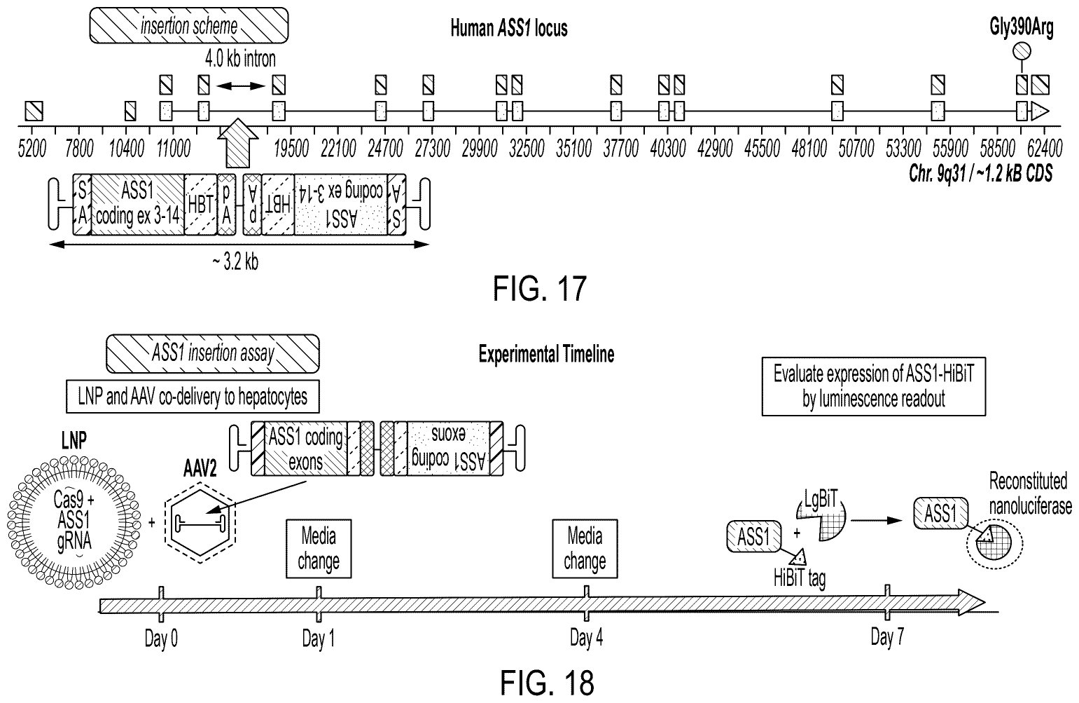

Human ASS1 guide RNAs were screened using primary human hepatocytes (PHH) for their activity to mediate insertion of a bidirectional ASS1 insertion template. Each half of the bidirectional insertion template contains an AAV ITR, followed by a splice acceptor sequence, followed by human ASS1 exons 3-14 coding sequence (either native ASS1 CDS or codon-altered CDS), followed by a HiBiT tag, followed by a stop codon, followed by a polyadenylation sequence. Specifically, the bidirectional ASS1 DNA gene insertion template comprises from 5′ to 3′: Alb splice acceptor (SEQ ID NO: 248), native human ASS1 exons 3-14 coding sequence (SEQ ID NO: 228, encoding SEQ ID NO: 230), G4S linker coding sequence, HiBiT tag coding sequence (SEQ ID NO: 245), bGH polyA (SEQ ID NO: 236), reverse complement of SV40 polyA (reverse complement of SEQ ID NO: 239), reverse complement of codon altered HiBiT tag coding sequence (reverse complement of SEQ ID NO: 246), reverse complement of G4S linker coding sequence, reverse complement of codon altered human ASS1 exons 3-14 coding sequence (reverse complement of SEQ ID NO: 229, encoding SEQ ID NO: 230), and reverse complement of Alb splice acceptor (reverse complement of SEQ ID NO: 248). The complete insertion construct (pAAV_ASS1_v4b_ins) including ITRs is set forth in SEQ ID NO: 233. Human ASS1 insertion template was packaged into AAV2 particles and co-administered with lipid nanoparticles (LNPs) containing Cas9 mRNA and human ASS1 guide RNA (gRNA). The 88 guide RNAs screened are shown in Table 20 (full sequences shown in Table 4, with modification pattern of SEQ ID NO: 429). Human ASS1 gRNAs were directed to target sites within the intron that lies between human ASS1 coding exons 2 and 3. The insertion scheme is shown in FIG. 17. Cells were incubated with AAV and LNP containing Cas9 mRNA and ASS1 gRNAs for 24 hours before receiving a media change on “day 1.” The LNPs were screened in 96-well format at 0.11 and 0.33 μg/ml in triplicate with 3E+4 MOI AAV (NGS % indel was also assessed at 0.33 μg/ml LNP). The ASS1 insertion assay is shown in FIG. 18. Three days later, cells received another media change on “day 4.” After an additional 3 day incubation, on “day 7” cells were lysed and incubated with LgBiT luminescence reagent, and luminescence was measured using a plate reader.

TABLE 20
Human ASS1 intron 2 guide RNAs screened.

| | | | Human Genomic Coordinates of |
|---|---|---|---|
| Guide RNA ID# | DNA-Targeting Segment | SEQ ID NO | Guide RNA Target Sequence |
| G035811 | GAGUGCGCUUUCAGCAGCGC | 31 | chr9: 130456020-130456040 |
| G035812 | CCUCUACACUCUAAUUUACG | 32 | chr9: 130456120-130456140 |
| G035813 | CAGCGGGGGUGGCCCCGUAU | 33 | chr9: 130458289-130458309 |
| G035814 | AGCUAGCUGAGACCUAUACG | 34 | chr9: 130458304-130458324 |
| G035815 | CCACGUAAAUUAGAGUGUAG | 35 | chr9: 130456123-130456143 |
| G035816 | CACUCGAAUCUUCAACACUC | 36 | chr9: 130456005-130456025 |
| G035817 | ACUCGAAUCUUCAACACUCA | 37 | chr9: 130456004-130456024 |
| G035818 | GUUUCACAGAACGGUGCAUC | 38 | chr9: 130457842-130457862 |
| G035819 | GGUUGCCACAACUACCAGGU | 39 | chr9: 130457676-130457696 |
| G035820 | UGGCCUUGUUCCCGUGUGGC | 40 | chr9: 130457579-130457599 |
| G035821 | CGGCCAGCCACACGGGAACA | 41 | chr9: 130457585-130457605 |
| G035822 | AAACAAGGCAUCACAUAGCG | 42 | chr9: 130457236-130457256 |
| G035823 | GUAUAGGUCUCAGCUAGCUG | 43 | chr9: 130458305-130458325 |
| G035824 | GGUGCCUUGAUUGUGAAUCU | 44 | chr9: 130455585-130455605 |
| G035825 | GUGCAUCAGGAUUGAUUAGC | 45 | chr9: 130457855-130457875 |
| G035826 | GGGGUUCCCAAUGACUUUUC | 46 | chr9: 130457508-130457528 |
| G035827 | UCAGCUAGCUGAGACCUAUA | 47 | chr9: 130458306-130458326 |
| G035828 | GAUGCACCGUUCUGUGAAAC | 48 | chr9: 130457842-130457862 |
| G035829 | GACGGUUUUGUCCCUGUUGG | 49 | chr9: 130457741-130457761 |
| G035830 | CUUGAAAUGUAGACCCCCAG | 50 | chr9: 130457527-130457547 |
| G035831 | AAGAAGCACCAGGUACAGCG | 51 | chr9: 130458274-130458294 |
| G035832 | UUGCCACAACUACCAGGUGG | 52 | chr9: 130457674-130457694 |
| G035833 | UUCUUGAAAUGUAGACCCCC | 53 | chr9: 130457529-130457549 |
| G035834 | AGCACCAGGUACAGCGGGGG | 54 | chr9: 130458278-130458298 |
| G035835 | UUAUGAUGAACAGUGUCAUC | 55 | chr9: 130455644-130455664 |
| G035836 | CAUGUUAAAAUGAACACGGG | 56 | chr9: 130455843-130455863 |
| G035837 | GAAGAGACACUCCACCUGGA | 57 | chr9: 130454575-130454595 |
| G035838 | UCUGCUCUCCAGCACUCGGG | 58 | chr9: 130455942-130455962 |
| G035839 | GUCUGAGCAGUAGUCCAGGA | 59 | chr9: 130455968-130455988 |
| G035840 | AUCUGGCAUACAGAGCCCCA | 60 | chr9: 130458332-130458352 |
| G035841 | CACAACUACCAGGUGGGGGG | 61 | chr9: 130457670-130457690 |
| G035842 | AGAGUAACAGUGCAGUGUGC | 62 | chr9: 130458217-130458237 |
| G035843 | UUUAAGAGUCGAGAAAUUCU | 63 | chr9: 130456631-130456651 |
| G035844 | GUUUUGUCCCUGUUGGGGGU | 64 | chr9: 130457737-130457757 |
| G035845 | GUGGCUGGCCGUCCUGUGCA | 65 | chr9: 130457594-130457614 |
| G035846 | CAUCUGGCAUACAGAGCCCC | 66 | chr9: 130458333-130458353 |
| G035847 | CUGUGGCCAGUAGGUGACUU | 67 | chr9: 130455885-130455905 |
| G035848 | UCUUGCAUAUAACAUUAGGA | 68 | chr9: 130456161-130456181 |
| G035849 | AUGAAGGGGCACAAGUGGCA | 69 | chr9: 130455707-130455727 |
| G035850 | AAAAGUCAUUGGGAACCCCU | 70 | chr9: 130457509-130457529 |
| G035851 | GUGGAGGGAUUAAUGACAGA | 71 | chr9: 130455330-130455350 |
| G035852 | CUGCAGAAGCACCCCACAGA | 72 | chr9: 130455805-130455825 |
| G035853 | AGGCAAGGACAGGGGCCAUC | 73 | chr9: 130458349-130458369 |
| G035854 | UCUGGCAUACAGAGCCCCAG | 74 | chr9: 130458331-130458351 |
| G035855 | GAAGAAGUAGGCAAGGACAG | 75 | chr9: 130458357-130458377 |
| G035856 | AGAACCAUUCCUCUGGAUCG | 76 | chr9: 130457545-130457565 |
| G035857 | CAGCUAGCUGAGACCUAUAC | 77 | chr9: 130458305-130458325 |
| G035858 | GGUUGUCUGAGCAGUAGUCC | 78 | chr9: 130455964-130455984 |
| G035859 | GAAACCCCGAUCCAGAGGAA | 79 | chr9: 130457552-130457572 |
| G035860 | UCACAUAGCGAGGAAGCCCU | 80 | chr9: 130457226-130457246 |
| G035861 | CAGAGAACUCCAAUUGAGUC | 81 | chr9: 130455564-130455584 |
| G035862 | GGGACAAAACCGUCUCUGGU | 82 | chr9: 130457747-130457767 |
| G035863 | AUCACAUAGCGAGGAAGCCC | 83 | chr9: 130457227-130457247 |
| G035864 | AUAAAUGCCCUGUAUGGGAC | 84 | chr9: 130455628-130455648 |
| G035865 | AGACGGUUUUGUCCCUGUUG | 85 | chr9: 130457742-130457762 |
| G035866 | AAAUGCCCUGUAUGGGACAG | 86 | chr9: 130455626-130455646 |
| G035867 | UGAUUGUGAAUCUAGGCAUG | 87 | chr9: 130455592-130455612 |
| G035868 | GAGACGGUUUUGUCCCUGUU | 88 | chr9: 130457743-130457763 |
| G035869 | ACUGAGAAACCCCGAUCCAG | 89 | chr9: 130457557-130457577 |
| G035870 | CAUAGCGAGGAAGCCCUGGG | 90 | chr9: 130457223-130457243 |
| G035871 | ACAUUCUAGUUCCAGGCCUG | 91 | chr9: 130455902-130455922 |
| G035872 | ACUCGGGAGGCACAGAACCA | 92 | chr9: 130455929-130455949 |
| G035873 | CAUGCCUAGAUUCACAAUCA | 93 | chr9: 130455592-130455612 |
| G035874 | GGAACUAGAAUGUCCCUCCU | 94 | chr9: 130455909-130455929 |
| G035875 | CUCAGCUAGCUGAGGCCCCU | 95 | chr9: 130458313-130458333 |
| G035876 | AAGAGACACUCCACCUGGAU | 96 | chr9: 130454574-130454594 |
| G035877 | GAAAAGUCAUUGGGAACCCC | 97 | chr9: 130457508-130457528 |
| G035878 | GCCAGUAGGUGACUUGGGAC | 98 | chr9: 130455880-130455900 |
| G035879 | AUCAAGGCACCAGACUCAAU | 99 | chr9: 130455576-130455596 |
| G035880 | AUCAGGAUUGAUUAGCAGGC | 100 | chr9: 130457859-130457879 |
| G035881 | UGCCACAACUACCAGGUGGG | 101 | chr9: 130457673-130457693 |
| G035882 | GACACUCCACCUGGAUGGGA | 102 | chr9: 130454570-130454590 |
| G035883 | GCAGACCCCUGUCCCAUACA | 103 | chr9: 130455618-130455638 |
| G035884 | GGGAUCAGCUCUGACCCUGC | 104 | chr9: 130455784-130455804 |
| G035885 | UCAGCUAGCUGAGGCCCCUG | 105 | chr9: 130458314-130458334 |
| G035886 | CUGGGGCUCUGUAUGCCAGA | 106 | chr9: 130458331-130458351 |
| G035887 | GAGUAACAGUGCAGUGUGCU | 107 | chr9: 130458218-130458238 |
| G035888 | GUGCUUCUGCAGGGUUGCCA | 108 | chr9: 130455813-130455833 |
| G035889 | UCAGAGCUGAUCCCCUCUGU | 109 | chr9: 130455790-130455810 |
| G035890 | CGGGAGGCACAGAACCAAGG | 110 | chr9: 130455926-130455946 |
| G035891 | UGUCUCCCUGAAAAGUCAUU | 111 | chr9: 130457499-130457519 |
| G035892 | AAAGUCAUUGGGAACCCCUG | 112 | chr9: 130457510-130457530 |
| G035893 | GUGGUGGAUGCUCCUGCCCC | 113 | chr9: 130454429-130454449 |
| G035894 | GUGGAAUUCUGGCAUCUAGU | 114 | chr9: 130457651-130457671 |
| G035895 | CCUGUGGCCAGUAGGUGACU | 115 | chr9: 130455886-130455906 |
| G035896 | AUGGAUAGAAAAACGGUGAA | 116 | chr9: 130455426-130455446 |
| G035897 | CUCCACCUGGAUGGGAAGGU | 117 | chr9: 130454566-130454586 |
| G035898 | ACUCCACCUGGAUGGGAAGG | 118 | chr9: 130454567-130454587 |

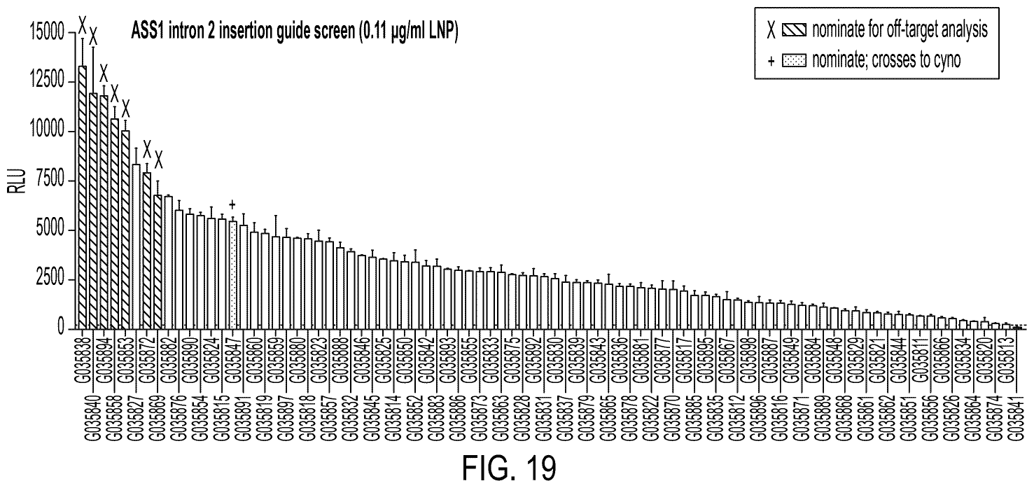

Insertion template pAAV_ASS1_v4b_ins was packaged into AAV2 and administered at a multiplicity of infection (MOI) of 3.0E+4 viral genomes per cell. LNP was co-administered at 0.11 μg/mL or 0.33 μg/mL. Day 7 HiBiT luminescence readings for 0.11 μg/mL LNP are plotted as a bar graph in FIG. 19, with lead gRNAs depicted as red (non-cynomolgus crossers) or blue (cynomolgus crosser) bars. Background luminescence signal observed when AAV2 insertion template was administered in the absence of ASS1 intron 2 gRNA LNP is depicted as a dashed horizontal line. Day 7 HiBiT luminescence readings for 0.11 and 0.33 μg/mL LNP were plotted as a heat map (data not shown), and the data indicated that the screening assay had a high signal-to-noise ratio and that the HiBiT luminescence signal was dependent upon Cas9 activity directed to ASS1 intron 2 gRNA. The data also indicated a wide range of ASS1 intron 2 gRNA potency and that the assay employed enabled the identification of high potency gRNAs. The significance of this is that greater gRNA potency is likely to translate to greater in vivo efficacy at a lower dose.

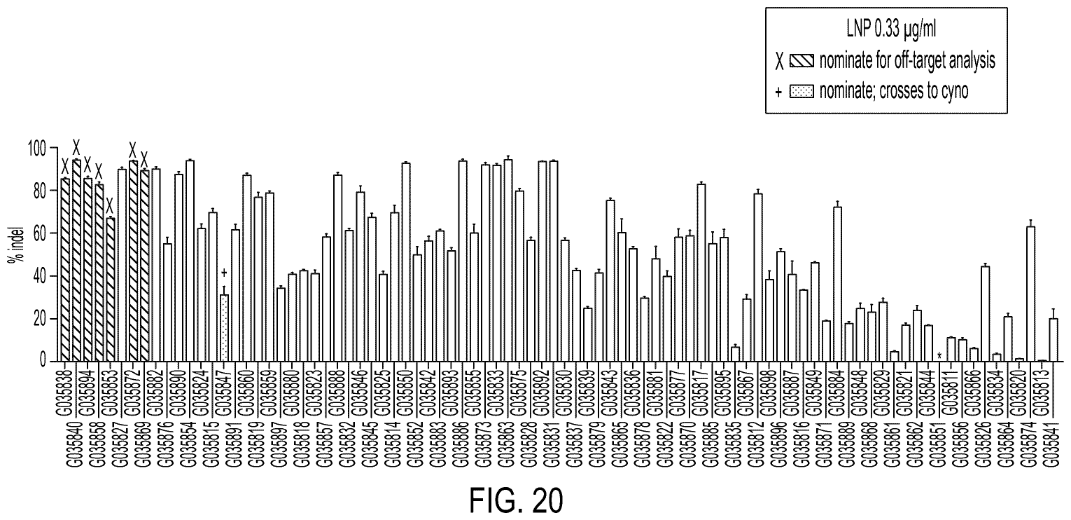

Short-read Illumina-based sequencing was used to determine the editing efficiency of human ASS1 intron 2 gRNAs. Specifically, PCR amplicons spanning each of the human ASS1 intron 2 gRNA target sites were produced from genomic DNA isolated from PHH cells treated with 0.33 μg/mL LNP. Sequencing reads containing an insertion or deletion (indel) 20 bp upstream or downstream from the gRNA target site was scored as an editing event. Editing efficiency was calculated as the percentage of edited reads relative to the total number of reads spanning the gRNA target site. As shown in FIG. 20, editing efficiency for each gRNA was plotted as a bar graph in the same order as the insertion luminescence bar graph from FIG. 19. The data qualitatively depicted that insertion luminescence and gRNA editing efficiency did not correlate well. The significance of these data is that gRNA editing efficiency cannot be used as an accurate proxy for gRNA-dependent AAV gene insertion.

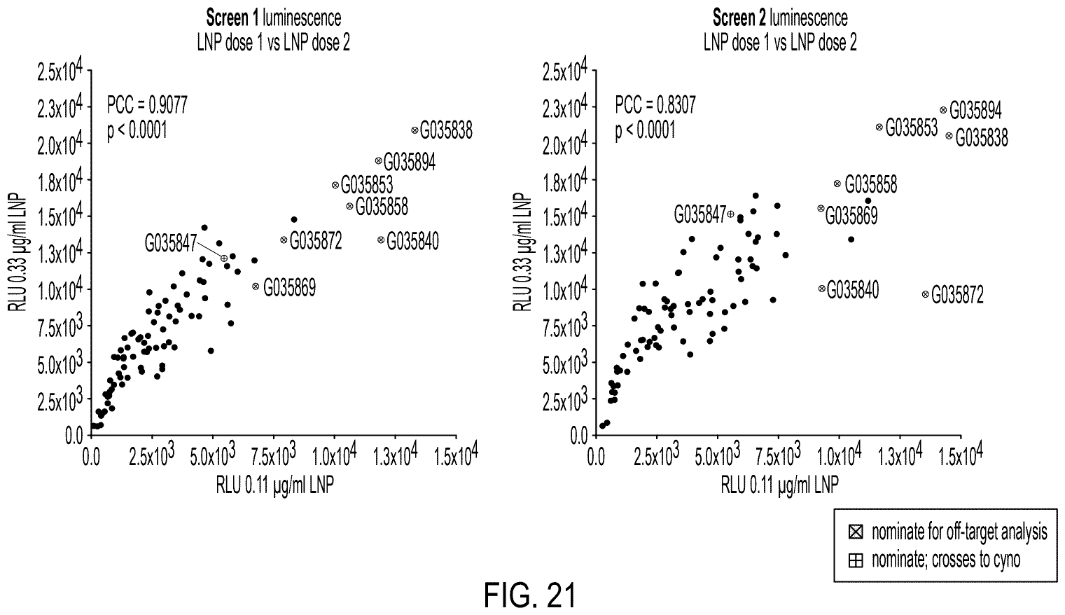

The in vitro ASS1 intron 2 gRNA screen depicted in FIG. 18 was performed on two separate occasions. Each of the two screens was conducted at both 0.11 and 0.33 μg/mL LNP. To determine the correlation between LNP doses, ASS1-HiBiT luminescence values obtained using 0.33 μg/ml LNP (y-axis) were plotted against the corresponding 0.11 μg/mL LNP (x-axis) luminescence values for each gRNA. See FIG. 21. The results from screen 1 are plotted on the left, while the results from screen 2 are plotted on the right. Pearson correlation coefficients (PCC) and statistical significance (p) are indicated. The data demonstrated good correlation between the two LNP doses evaluated, indicating dose responsiveness of the LNP with respect to ASS1 insertion. The significance of this correlation is that the LNP doses that were evaluated are likely in the linear range of the insertion assay, thus facilitating proper rank ordering of gRNA potency.

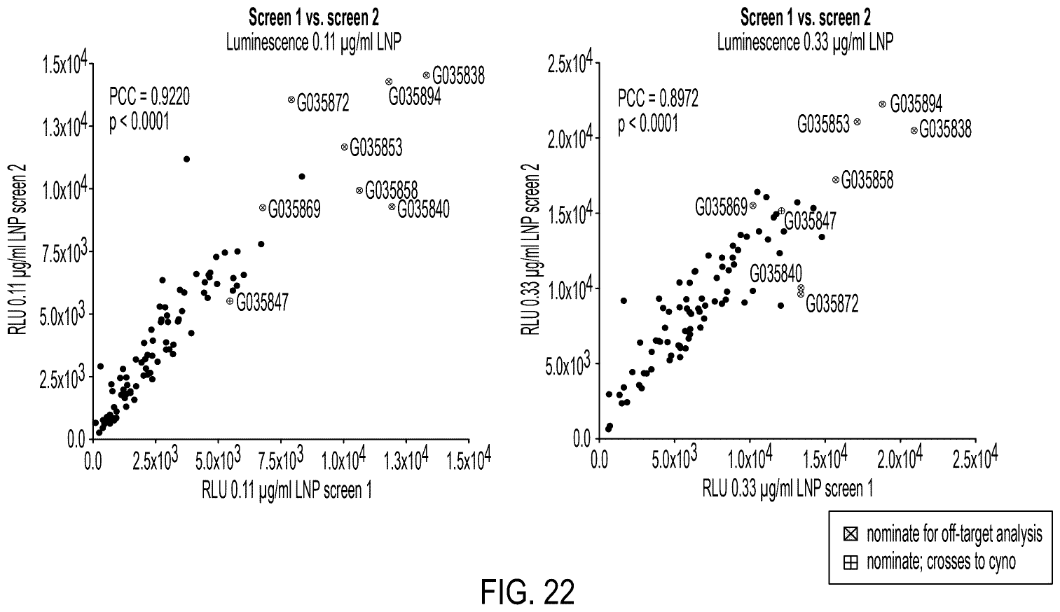

To determine the correlation between screens 1 and 2, ASS1-HiBiT luminescence values for the same LNP dose were plotted. See FIG. 22. The plot on the left depicts luminescence values for each gRNA at the 0.11 μg/mL dose from screen 1 (x-axis) and screen 2 (y-axis). The plot on the right depicts luminescence values for each gRNA at the 0.33 μg/mL dose from screen 1 (x-axis) and screen 2 (y-axis). Pearson correlation coefficients (PCC) and statistical significance (p) are indicated. The data demonstrated strong correlation between screens performed on different days, and demonstrated that the data sets are robust.

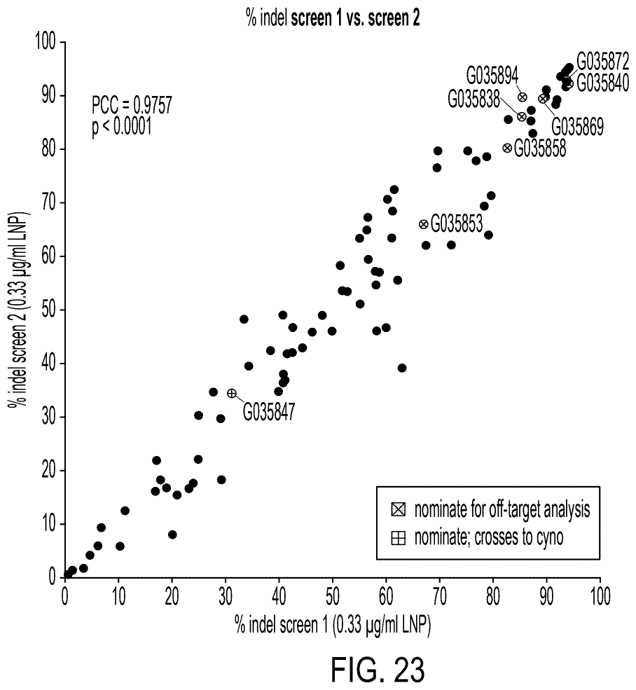

To determine the indel frequency correlation between screens, indel frequencies from screens 1 and 2 for each gRNA were plotted. See FIG. 23. Each dot on the scatter plot indicates the indel frequency for an individual gRNA from screen 1 (x-axis) and screen 2 (y-axis). Pearson correlation coefficients (PCC) and statistical significance (p) are indicated. The data demonstrated strong correlation between screens performed on different days, and demonstrated that the data sets are robust.

A summary table is shown in Table 21 indicating the top 30 intron 2 ASS1 gRNAs as determined by their ASS1-HiBiT luminescence values. Indel frequency is also denoted.

TABLE 21
Human ASS1 intron 2 guide RNA screen data summary.

| | Screen 1 | Screen 2 | Screen 1 | Screen 2 | | |
|---|---|---|---|---|---|---|
| | RLUs 0.11 | RLUs 0.11 | RLUs 0.33 | RLUs 0.33 | Screen 1% | Screen 2% |
| Guide | μg/mL LNP | μg/mL LNP | μg/mL LNP | μg/mL LNP | indel | indel |
| G035838 | 13304.00 | 14534.00 | 20909.33 | 20495.67 | 85.34 | 86.09 |
| G035894 | 11807.33 | 14279.33 | 18811.67 | 22255.00 | 85.48 | 89.74 |
| G035853 | 10037.33 | 11667.00 | 17131.67 | 21082.00 | 67.01 | 66.02 |
| G035840 | 11926.33 | 9288.67 | 13397.67 | 10030.33 | 94.16 | 92.25 |
| G035872 | 7914.67 | 13559.67 | 13383.33 | 9619.67 | 93.72 | 92.90 |
| G035858 | 10634.67 | 9936.67 | 15703.67 | 17225.33 | 82.67 | 80.25 |
| G035827 | 8339.33 | 10490.67 | 14782.33 | 13422.33 | 89.78 | 89.81 |
| G035869 | 6771.00 | 9249.67 | 10202.67 | 15516.67 | 89.28 | 89.46 |
| G035882 | 6713.00 | 7796.00 | 11980.00 | 12343.33 | 89.95 | 91.14 |
| G035890 | 5817.33 | 7429.33 | 12257.00 | 13786.00 | 87.42 | 82.99 |
| G035876 | 6019.00 | 6565 67 | 11206.33 | 13250.00 | 55.06 | 63.40 |
| G035891 | 5263.33 | 7454.33 | 13149.33 | 15725.00 | 61.53 | 72.50 |
| G035860 | 4918.33 | 7281.33 | 5781.33 | 9278.00 | 87.06 | 85.31 |
| G035824 | 5605.33 | 6436.00 | 8943.67 | 11595.00 | 62.17 | 55.58 |
| G035846 | 3741.33 | 11188.33 | 11098.67 | 16067.00 | 79.15 | 64.02 |
| G035859 | 4680.67 | 6655.67 | 9389.67 | 13555.67 | 78.80 | 78.64 |
| G035854 | 5745.33 | 6134.00 | 7670.33 | 9141.33 | 93.87 | 94.90 |
| G035815 | 5583.67 | 5936.33 | 11587.67 | 14721.33 | 69.68 | 79.72 |
| G035880 | 4615.67 | 6565.67 | 10498.00 | 16416.00 | 40.81 | 38.02 |
| G035897 | 4655.33 | 6468.33 | 14221.33 | 15340.00 | 34.35 | 39.53 |
| G035888 | 4126.67 | 6601.67 | 8177.33 | 11440.33 | 87.13 | 87.29 |
| G035819 | 4853.33 | 5936.00 | 11746.33 | 14937.33 | 76.81 | 77.87 |
| G035823 | 4461.33 | 6270.67 | 10613.00 | 13796.67 | 41.12 | 36.90 |
| G035847 | 5461.33 | 5518.67 | 12113.33 | 15164.00 | 31.16 | 34.43 |
| G035818 | 4576.00 | 5648.33 | 12059.33 | 8861.00 | 42.51 | 42.07 |
| G035857 | 4432.33 | 5853.33 | 8152.00 | 12044.67 | 58.23 | 46.11 |
| G035814 | 3472.00 | 5965.00 | 7799.67 | 10699.33 | 69.53 | 76.57 |
| G035845 | 3644.67 | 5864.00 | 8598.33 | 11206.67 | 67.46 | 62.07 |
| G035825 | 3554.33 | 5119.67 | 8875.67 | 12843.67 | 40.72 | 49.06 |
| G035875 | 2773.67 | 6357.00 | 8857.33 | 12052.00 | 79.64 | 71.36 |

G035838, G035894, G035853, G035840, G035872, G035858, G035869, and G035847 were chosen for off-target analysis. G035838 was the initial top performing gRNA overall, while G035847 was the top performing gRNA that fully matches to cynomolgus macaque.

Off-target site prediction was conducted for G035838, G035894, G035853, G035840, G035872, G035858, G035869, and G035847 using homology-based in silico Cas-OFFinder tool (Bae et al, Bioinformatics, 2014) and biochemical Cas9 cleavage assay SITE-Seq (Cameron et al, Nat Med, 2017). The number of predicted off-target sites provided an initial assessment for gRNA specificity. These assays do not provide confirmation of off-target editing at predicted off-target sites, but rather identify genomic target sites that may be prone to off-target editing and thus should be evaluated further via additional quantitative assays.

A summary table indicating the number of predicted off-target sites identified through SITE-seq and the in vitro gene insertion potency for ASS1 gRNAs G035838, G035894, G035853, G035840, G035872, G035858, G035869, and G035847 is provided below in Table 22. The SITE-seq column indicates the number of unique cleavage sites identified in high molecular weight genomic DNA produced by 64 nM Cas9-gRNA ribonucleoprotein. These target sites comprise the SITE-seq predicted off-target site profile for each respective gRNA. To determine gRNA EC50 potencies, in vitro gene insertion assays were performed on human hepatocytes as described in above in Example 5, using a dilution series of ASS1 gRNA LNP and fixed dose of AAV2 packaged HiBiT-tagged insertion template pAAV_ASS1_v4_ins. The results are shown in Table 22.

TABLE 22
Predicted Off-Target Sites and In Vitro Gene Insertion Potency

| | | LNP EC50 (ng/mL) with Insertion |
|---|---|---|
| Guide | SITE-seq Sites | Template pAAV_ASS1_v4_ins |
| G035872 | 754 | 42.18 |
| G035894 | 3165 | 46.65 |
| G035840 | 386 | 55.74 |
| G035869 | 215 | 73.00 |
| G035853 | 173 | 90.24 |
| G035838 | 298 | 103.8 |
| G035858 | 53 | 96.78 |
| G035847 | 128 | 123.6 |

ASS1 gRNAs G035840, G035869, and G035858 underwent further evaluation to determine if off-target editing could be detected at their respective predicted off-target sites. Two distinct primary human hepatocyte donor lines (Hu8287 and Hu8300) were treated with supersaturating doses (4×EC90) of ASS1 gRNA LNP. Editing at predicted off-target sites was assessed through the use of multiplex (rhAmpSeq) and singleplex amplicon next-generation sequencing (TOT-seq collectively). Of the 94 potential off-target sites identified through Cas-OFFinder and SITE-seq for G035858, none displayed reproducible editing in both hepatocyte lines. Lower limit of detection was established as 0.8% editing at 1,000-fold read coverage for 80% sensitivity. One potential off-target site could not be assessed due to sequencing difficulty. The results are shown in Table 23.

TABLE 23
TOT-Seq Analysis.

| Candidate sgRNA | G035840 | | G035869 | | G035858 | |
|---|---|---|---|---|---|---|
| Total # potential off-target sites | 434 | | 254 | | 94 | |
| evaluated via TOTseq | | | | | | |
| PHH ID | Hu8287 | Hu8300 | Hu8287 | Hu8300 | Hu8287 | Hu8300 |
| On-target editing (% indel) | 87.2% | 91.4% | 86.7% | 89.8% | 86.2% | 83.2% |
| # potential off-target sites | 431/434 | 430/434 | 237/254 | 227/254 | 93/94 | 93/94 |
| successfully characterized | | | | | | |
| # candidate off-target sites | 9 | 7 | 4 | 5 | 0 | 1 |
| identified in TOT-seq | (3 exonic) | (3 exonic) | (1 exonic) | (1 exonic) | | (0 exonic) |

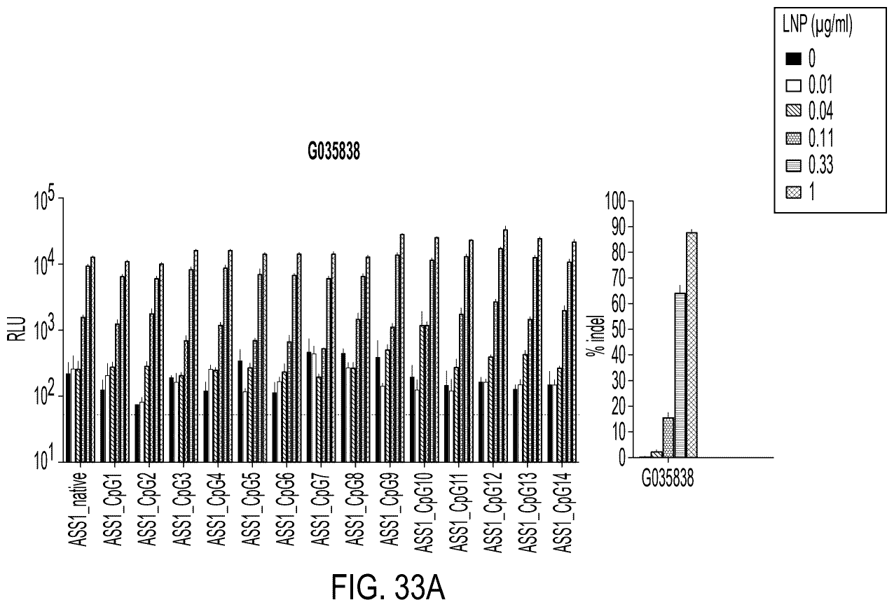

The efficiency of ASS1 intron 2 gene insertion was assessed in vivo in humanized ASS1^{WT/WT} knock-in mice. Adult humanized ASS1^{WT/WT} knock-in mice were dosed with 0.3 mg/kg G035840, G035869, or G035858 ASS1 gRNA LNP+1.5E11 vector genomes per mouse AAV8-ASS1-v4b. Two weeks after dosing, mice were euthanized, and livers were harvested. DNA insertion efficiency was measured from liver genomic DNA using the digital droplet PCR method described in FIG. 10. Insertion-derived ASS1 mRNA transcripts were measured from liver RNA using digital droplet qRT-PCR where for each sample ASS1 insertion-specific amplicons (forward primer in upstream exon, reverse primer in AAV insertion template) were normalized against total ASS1 amplicons (forward and reverse primers located in ASS1 exon sequence upstream of gRNA insertion site). These analyses revealed ˜15-20% liver insertion efficiency for G035858 at both the DNA and RNA level. See FIG. 32. G035858 was ˜10× more potent than the mAss1 gRNA used in the fold mouse experiments.

G035858 showed robust in vitro and in vivo insertion efficiency. It showed ˜17% liver DNA insertion frequency in ASS1 humanized mice at a dose of 0.3 mpk LNP, and 5-6% insertion is sufficient for long-term ammonia normalization in fold citrullinemia mice. It had a favorable off-target profile with minimal off-target effects, and a suitable NHP surrogate gRNA was identified (G040925) which exhibits similar insertion potency in non-human primate hepatocytes.

### Example 6. Human ASS1 Gene Insertion Guide RNA Screen (Intron 1)

The human ASS1 locus, specifically intron 1 between coding exons 1 and 2, was evaluated for the presence of the specific CRISPR/Cas9 protospacer adjacent motifs (PAM) sites to generate a list of target sites in the first intron of the locus where a Cas9-induced DNA cut could occur. The sites were cross-referenced against a single nucleotide polymorphism (SNP) analysis to ensure selected sites did not have common SNPs that might impact gRNA efficiency. A list of sgRNAs was then generated, and guide RNAs were synthesized and formulated into LNPs with Cas9 mRNA for evaluation in vitro and in vivo.

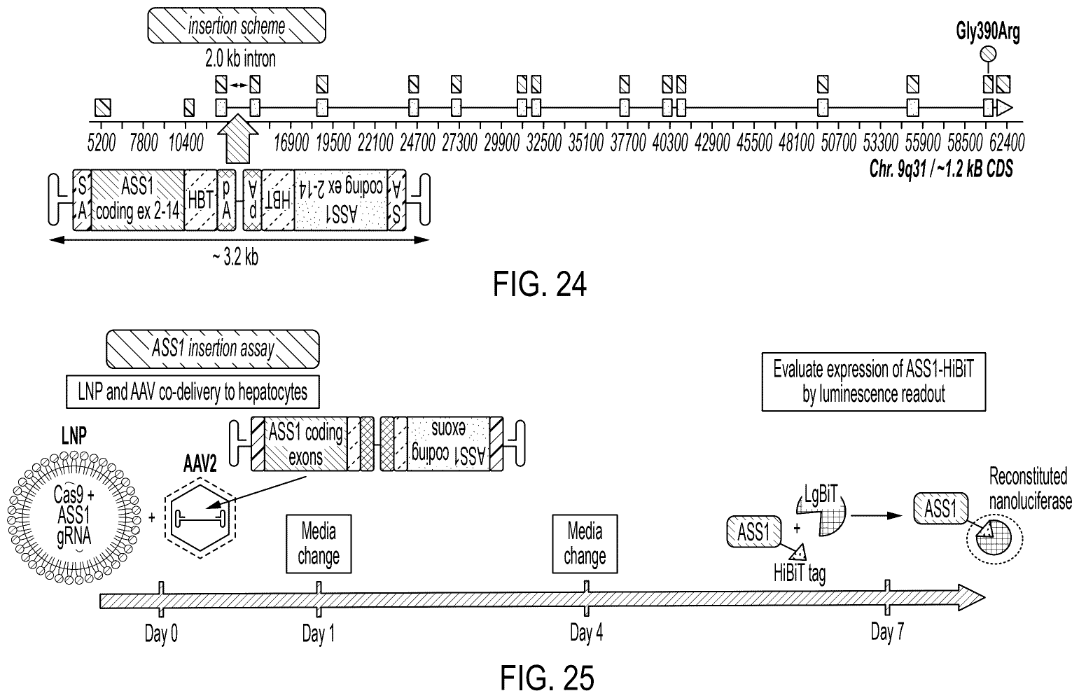

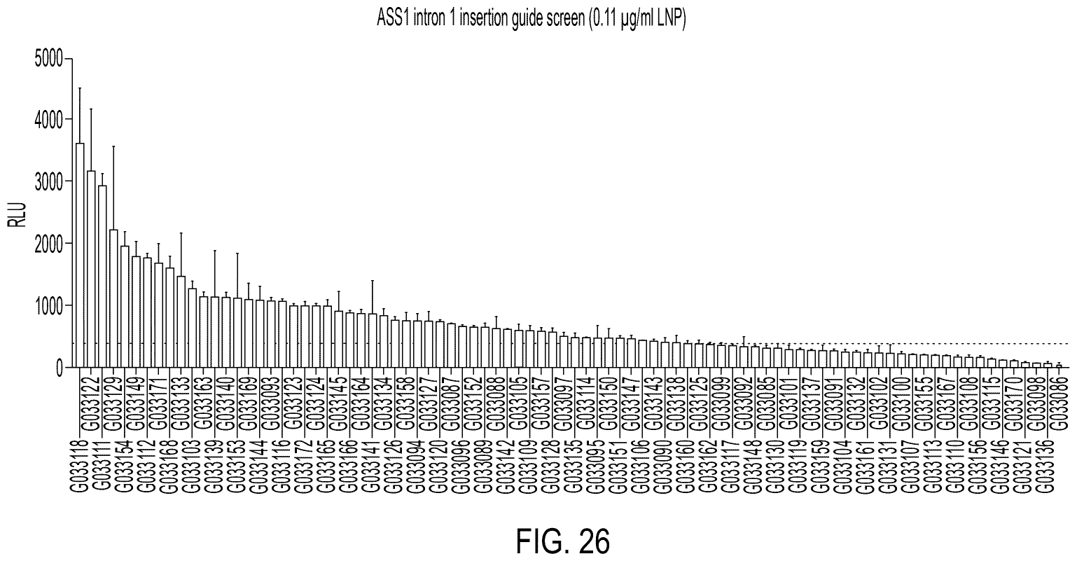

Human ASS1 guide RNAs were screened using primary human hepatocytes (PHH) for their activity to mediate insertion of a bidirectional ASS1 insertion template (pAAV_ASS1_v2_ins). Each half of the bidirectional insertion template contains an AAV ITR, followed by a splice acceptor sequence, followed by human ASS1 exons 2-14 coding sequence (either native ASS1 CDS or codon-altered CDS), followed by a HiBiT tag, followed by a stop codon, followed by a polyadenylation sequence. Specifically, the bidirectional ASS1 DNA gene insertion template comprises from 5′ to 3′: Alb splice acceptor (SEQ ID NO: 248), native human ASS1 exons 2-14 coding sequence (SEQ ID NO: 695, encoding SEQ ID NO: 227), G4S linker coding sequence, HiBiT tag coding sequence (SEQ ID NO: 245), bGH polyA (SEQ ID NO: 236), reverse complement of SV40 polyA (reverse complement of SEQ ID NO: 239), reverse complement of codon altered HiBiT tag coding sequence (reverse complement of SEQ ID NO: 246), reverse complement of G4S linker coding sequence, reverse complement of codon altered human ASS1 exons 2-14 coding sequence (reverse complement of SEQ ID NO: 696, encoding SEQ ID NO: 227), and reverse complement of Alb splice acceptor (reverse complement of SEQ ID NO: 248). The complete insertion construct (pAAV_ASS1_v2_ins) including ITRs is set forth in SEQ ID NO: 694. Human ASS1 insertion template was packaged into AAV2 particles and co-administered with lipid nanoparticles (LNPs) containing Cas9 mRNA and human ASS1 guide RNA (gRNA). The 88 guide RNAs screened are shown in Table 22 (full sequences shown in Table 6, with modification pattern of SEQ ID NO: 429). Human ASS1 gRNAs were directed to target sites within the intron that lies between human ASS1 coding exons 1 and 2. The insertion scheme is shown in FIG. 24. Cells were incubated with AAV and LNP containing Cas9 mRNA and ASS1 gRNAs for 24 hours before receiving a media change on “day 1.” The LNPs were screened in 96-well format at 0.11 and 0.33 μg/ml in triplicate with 3E+4 MOI AAV (NGS % indel was also assessed at 0.33 μg/ml LNP). The ASS1 insertion assay is shown in FIG. 25. Three days later, cells received another media change on “day 4.” After an additional 3 day incubation, on “day 7” cells were lysed and incubated with LgBiT luminescence reagent, and luminescence was measured using a plate reader.

TABLE 22
Human ASS1 intron 1 guide RNAs screened.

| | | | Human Genomic Coordinates of |
|---|---|---|---|
| Guide RNA ID# | DNA-Targeting Segment | SEQ ID NO | Guide RNA Target Sequence |
| G033085 | CAUAGGGACUAAUGCGUGGU | 430 | chr9: 130453191-130453211 |
| G033086 | CACCGUCACUCAUUCAAGCC | 431 | chr9: 130452430-130452450 |
| G033087 | GUGGCCAGGUUCAGUCGAAG | 432 | chr9: 130452865-130452885 |
| G033088 | AAUGCGUGGUGGGUCCCCAU | 433 | chr9: 130453181-130453201 |
| G033089 | AGCUCCUCUUCGACUGAACC | 434 | chr9: 130452858-130452878 |
| G033090 | CCAUAGGGACUAAUGCGUGG | 435 | chr9: 130453192-130453212 |
| G033091 | CAGCCAUAGGGACUAAUGCG | 436 | chr9: 130453195-130453215 |
| G033092 | UCAUUCGGCUCACCAGACCC | 437 | chr9: 130453672-130453692 |
| G033093 | GGUCUGGUGAGCCGAAUGAA | 438 | chr9: 130453673-130453693 |
| G033094 | UGGCUUGUUUGCCGGCAUAU | 439 | chr9: 130453378-130453398 |
| G033095 | AGGAGGGCUUACCCCCAGUA | 440 | chr9: 130453155-130453175 |
| G033096 | GCCGGCAAACAAGCCACUUU | 441 | chr9: 130453374-130453394 |
| G033097 | GAGCUGGGCGUAUAGACCCC | 442 | chr9: 130453575-130453595 |
| G033098 | GCAUCAUGGGGUGCGAGGCU | 443 | chr9: 130453214-130453234 |
| G033099 | AGCUGGGCGUAUAGACCCCU | 444 | chr9: 130453576-130453596 |
| G033100 | UAAUGCGUGGUGGGUCCCCA | 445 | chr9: 130453182-130453202 |
| G033101 | AAGGAGGGCUUACCCCCAGU | 446 | chr9: 130453154-130453174 |
| G033102 | CAUCAUGGGGUGCGAGGCUU | 447 | chr9: 130453215-130453235 |
| G033103 | UCUCGAAGCCUGUCUUGAAC | 448 | chr9: 130452460-130452480 |
| G033104 | UUGGGAGCUGGAUUGUAGCG | 449 | chr9: 130454098-130454118 |
| G033105 | GGGUCUGGUGAGCCGAAUGA | 450 | chr9: 130453672-130453692 |
| G033106 | AGUCCCUAUGGCUGCAUCAU | 451 | chr9: 130453201-130453221 |
| G033107 | UGGGGGUAAGCCCUCCUUUU | 452 | chr9: 130453152-130453172 |
| G033108 | UUACCCCCAGUAGGGCCCAU | 453 | chr9: 130453163-130453183 |
| G033109 | UUGAGAGGGAUGACUCCUAU | 454 | chr9: 130452835-130452855 |
| G033110 | GGGGGUAAGCCCUCCUUUUU | 455 | chr9: 130453151-130453171 |
| G033111 | UGACAAGCUAAUCUUUGCAC | 456 | chr9: 130453923-130453943 |
| G033112 | GCCCAAUGCCCUUGUCAUUC | 457 | chr9: 130453414-130453434 |
| G033113 | UACCCCCAGUAGGGCCCAUG | 458 | chr9: 130453164-130453184 |
| G033114 | CCUAGUCAAAGGCCAUGCCU | 459 | chr9: 130453876-130453896 |
| G033115 | CACAGCGGGGCAAUCAGAGC | 460 | chr9: 130453553-130453573 |
| G033116 | UAGACCCCUGGGCUACCACC | 461 | chr9: 130453587-130453607 |
| G033117 | AUCCCGCCCACUGAAGGAGU | 462 | chr9: 130453248-130453268 |
| G033118 | UGCAGGCUGACAGCAUACUC | 463 | chr9: 130452692-130452712 |
| G033119 | CAGAACCAGGUGAUCCCCCA | 464 | chr9: 130452754-130452774 |
| G033120 | GGGUGUAUCCAUCUCACUGU | 465 | chr9: 130453527-130453547 |
| G033121 | GGGUCCCCAUGGGCCCUACU | 466 | chr9: 130453171-130453191 |
| G033122 | CAAGGCAUGGCCUUUGACUA | 467 | chr9: 130453874-130453894 |
| G033123 | GAUCUAUCUCCAACUCUACU | 468 | chr9: 130453063-130453083 |
| G033124 | CAGUGAGAUGGAUACACCCU | 469 | chr9: 130453526-130453546 |
| G033125 | GAAGAAAUCCCGCCCACUGA | 470 | chr9: 130453254-130453274 |
| G033126 | AUCCCCCAGGUGGUAGCCCA | 471 | chr9: 130453595-130453615 |
| G033127 | GUCCCCAUGGGCCCUACUGG | 472 | chr9: 130453169-130453189 |
| G033128 | AAUCCCCCAGGUGGUAGCCC | 473 | chr9: 130453596-130453616 |
| G033129 | CAUGAGCCUACUCCUUCAGU | 474 | chr9: 130453239-130453259 |
| G033130 | UCCUAGUUCCAGUUCAAGAC | 475 | chr9: 130452471-130452491 |
| G033131 | GGUCCCCAUGGGCCCUACUG | 476 | chr9: 130453170-130453190 |
| G033132 | CCACCACGCAUUAGUCCCUA | 477 | chr9: 130453189-130453209 |
| G033133 | UGGGUGGACCCCAUUCCACA | 478 | chr9: 130453285-130453305 |
| G033134 | AGUUAGACAUGCCAAUAUGC | 479 | chr9: 130453392-130453412 |
| G033135 | AGUGCAAAGAUUAGCUUGUC | 480 | chr9: 130453924-130453944 |
| G033136 | UGGGUCCCCAUGGGCCCUAC | 481 | chr9: 130453172-130453192 |
| G033137 | AUGCCCUUGUCAUUCAGGUU | 482 | chr9: 130453419-130453439 |
| G033138 | UAGUCCCUAUGGCUGCAUCA | 483 | chr9: 130453200-130453220 |
| G033139 | GCCUGUCUUGAACUGGAACU | 484 | chr9: 130452467-130452487 |
| G033140 | CAACCAUCUGCUGUCAUUGC | 485 | chr9: 130454004-130454024 |
| G033141 | ACAGUGAGAUGGAUACACCC | 486 | chr9: 130453527-130453547 |
| G033142 | UUUGAGAGGGAUGACUCCUA | 487 | chr9: 130452836-130452856 |
| G033143 | GCCAAAAGUGGCUUGUUUGC | 488 | chr9: 130453370-130453390 |
| G033144 | GAUUGUAGCGUGGUGAUGGC | 489 | chr9: 130454088-130454108 |
| G033145 | CCACUGAAGGAGUAGGCUCA | 490 | chr9: 130453241-130453261 |
| G033146 | GGACAGGUUGCAGGACACGC | 491 | chr9: 130452364-130452384 |
| G033147 | GCCUGAAGCCAUCCUACCCC | 492 | chr9: 130453019-130453039 |
| G033148 | UCACUGUAGGUAGCACAGCG | 493 | chr9: 130453540-130453560 |
| G033149 | CCAAGGCAUGGCCUUUGACU | 494 | chr9: 130453873-130453893 |
| G033150 | ACCUGAAUGACAAGGGCAUU | 495 | chr9: 130453418-130453438 |
| G033151 | CCAUGAGCCUACUCCUUCAG | 496 | chr9: 130453238-130453258 |
| G033152 | AAACAUGGCAGUGUCUAGAC | 497 | chr9: 130453748-130453768 |
| G033153 | GGGAUGACUCCUAUGGGCCC | 498 | chr9: 130452829-130452849 |
| G033154 | GCUUCCAUGGGUUGGAGCAU | 499 | chr9: 130454063-130454083 |
| G033155 | CUUACCCCCAGUAGGGCCCA | 500 | chr9: 130453162-130453182 |
| G033156 | GUGAACUCAGGGCUCCCCCC | 501 | chr9: 130454248-130454268 |
| G033157 | GUCCCUAUGGCUGCAUCAUG | 502 | chr9: 130453202-130453222 |
| G033158 | GGCAUGGCCUUUGACUAGGG | 503 | chr9: 130453877-130453897 |
| G033159 | GCACCCCAUGAUGCAGCCAU | 504 | chr9: 130453208-130453228 |
| G033160 | AUGAGUGACGGUGAGCAGAC | 505 | chr9: 130452423-130452443 |
| G033161 | UGGAAGAAUCUCACCUCUCC | 506 | chr9: 130454128-130454148 |
| G033162 | CUGGGUGGACCCCAUUCCAC | 507 | chr9: 130453286-130453306 |
| G033163 | UGACAGCAUACUCUGGAGAU | 508 | chr9: 130452685-130452705 |
| G033164 | CAGCUCUCCUCCCUAGUCAA | 509 | chr9: 130453887-130453907 |
| G033165 | CCAGAACCAGGUGAUCCCCC | 510 | chr9: 130452755-130452775 |
| G033166 | AGCCAGGUGAUGGAGGCGCG | 511 | chr9: 130452560-130452580 |
| G033167 | CCUUUCCUUUUUGCGUAAGU | 512 | chr9: 130452961-130452981 |
| G033168 | UCUGGAAGGCAUCUCAAGGA | 513 | chr9: 130452541-130452561 |
| G033169 | AACAUGGCAGUGUCUAGACA | 514 | chr9: 130453749-130453769 |
| G033170 | UGAACUCAGGGCUCCCCCCA | 515 | chr9: 130454249-130454269 |
| G033171 | AGACCCCUGGGCUACCACCU | 516 | chr9: 130453588-130453608 |
| G033172 | UCAGCCAAUGCUCCAACCCA | 517 | chr9: 130454056-130454076 |

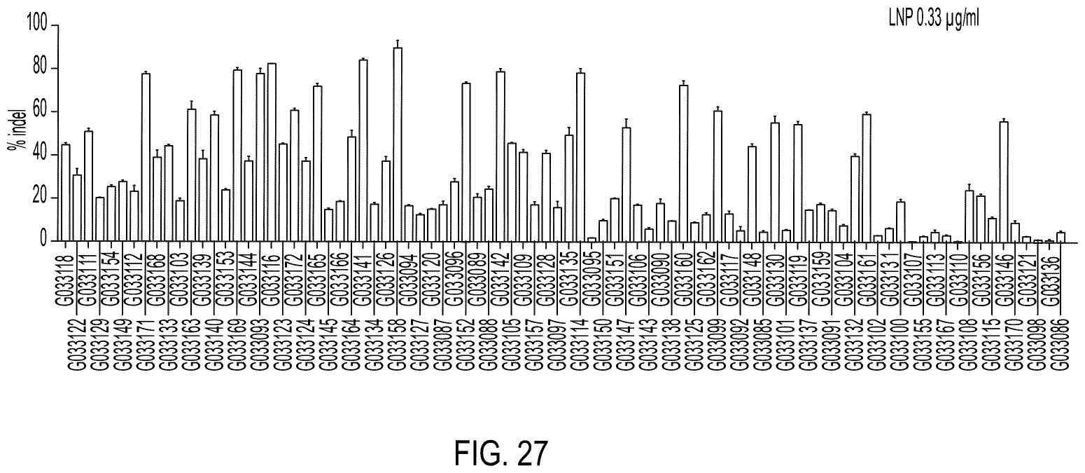

Insertion template pAAV_ASS1_v2_ins was packaged into AAV2 and administered at a multiplicity of infection (MOI) of 3.0E+4 viral genomes per cell. LNP was co-administered at 0.11 μg/mL or 0.33 μg/mL. Day 7 HiBiT luminescence readings for 0.11 μg/mL LNP are plotted as a bar graph in FIG. 26. Background luminescence signal observed when AAV2 insertion template was administered in the absence of ASS1 intron 1 gRNA LNP is depicted as a dashed horizontal line. Day 7 HiBiT luminescence readings for 0.11 and 0.33 μg/mL LNP were plotted as a heat map (data not shown). The data also indicated a range of ASS1 intron 1 gRNA potency and that the assay employed enabled the identification of high potency gRNAs. The significance of this is that greater gRNA potency is likely to translate to greater in vivo efficacy at a lower dose.

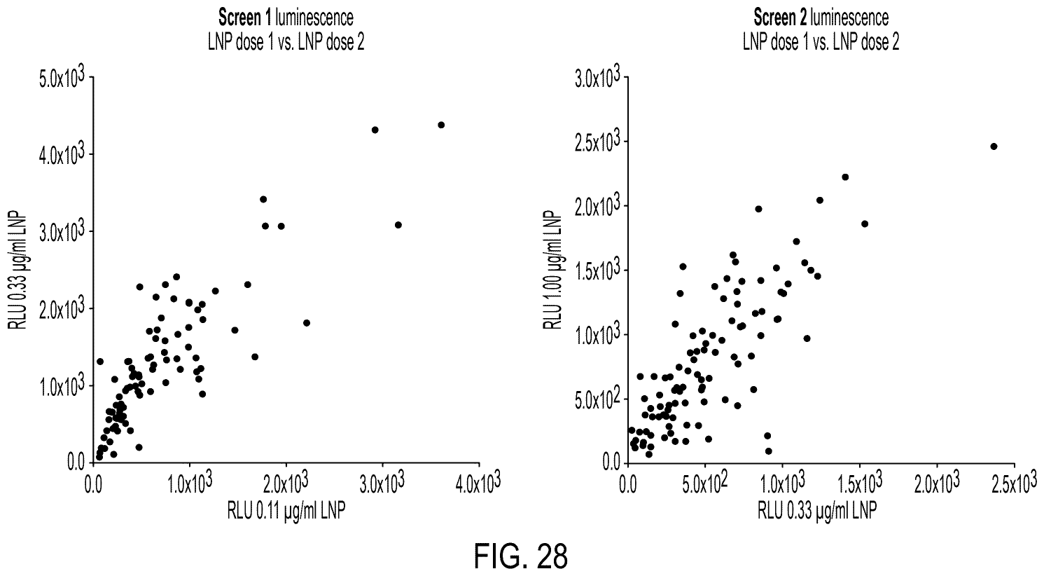

Short-read Illumina-based sequencing was used to determine the editing efficiency of human ASS1 intron 1 gRNAs. Specifically, PCR amplicons spanning each of the human ASS1 intron 1 gRNA target sites were produced from genomic DNA isolated from PHH cells treated with 0.33 μg/mL LNP. Sequencing reads containing an insertion or deletion (indel) 20 bp upstream or downstream from the gRNA target site was scored as an editing event. Editing efficiency was calculated as the percentage of edited reads relative to the total number of reads spanning the gRNA target site. As shown in FIG. 27, editing efficiency for each gRNA was plotted as a bar graph in the same order as the insertion luminescence bar graph from FIG. 26. The data qualitatively depicted that insertion luminescence and gRNA editing efficiency did not correlate well. The significance of these data is that gRNA editing efficiency cannot be used as an accurate proxy for gRNA-dependent AAV gene insertion.

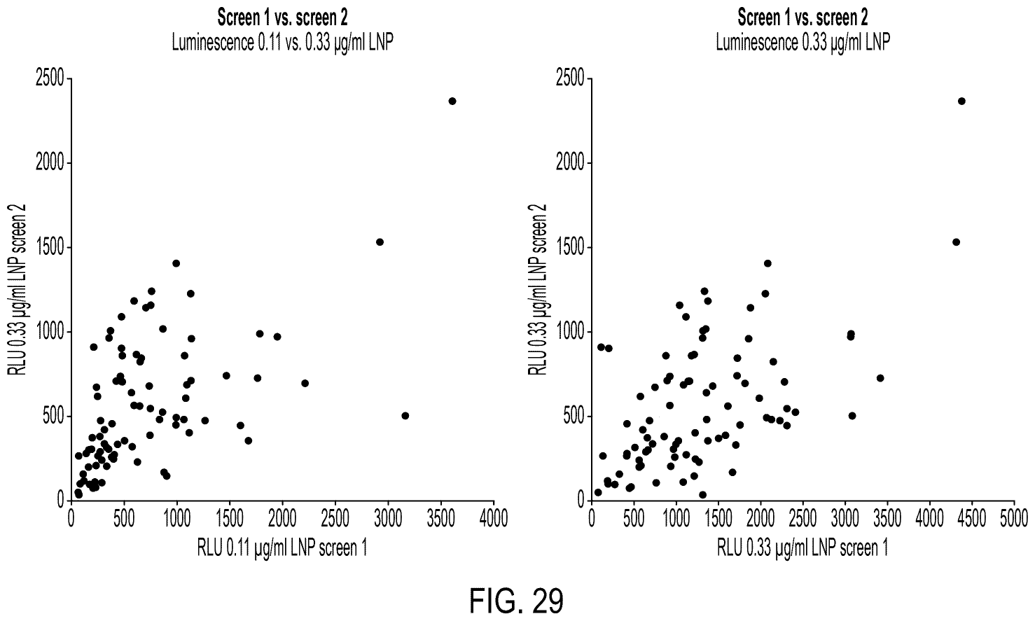

The in vitro ASS1 intron 1 gRNA screen depicted in FIG. 25 was performed on two separate occasions. Each of the two screens was conducted at both 0.11 and 0.33 μg/mL LNP. To determine the correlation between LNP doses, ASS1-HiBiT luminescence values obtained using 0.33 μg/ml LNP (y-axis) were plotted against the corresponding 0.11 μg/mL LNP (x-axis) luminescence values for each gRNA. See FIG. 28. The results from screen 1 are plotted on the left, while the results from screen 2 are plotted on the right. Pearson correlation coefficients (PCC) and statistical significance (p) are indicated. The data demonstrated good correlation between the two LNP doses evaluated, indicating dose responsiveness of the LNP with respect to ASS1 insertion. The significance of this correlation is that the LNP doses that were evaluated are likely in the linear range of the insertion assay, thus facilitating proper rank ordering of gRNA potency.

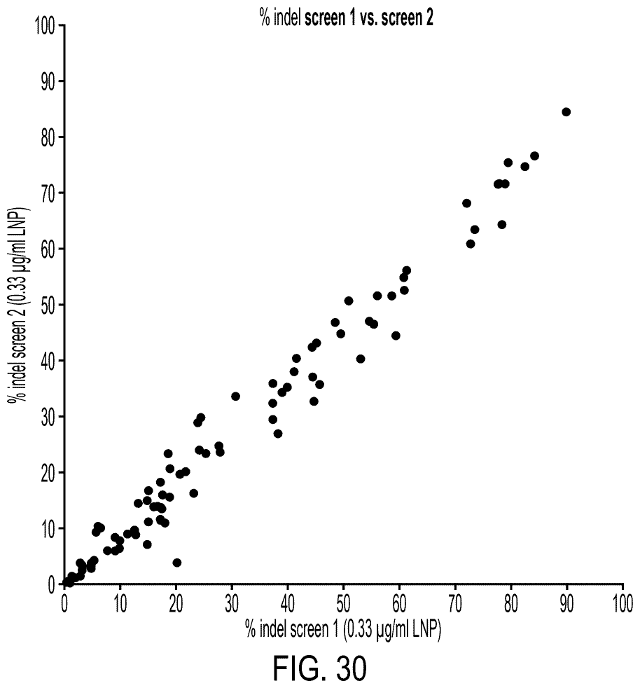

To determine the correlation between screens 1 and 2, ASS1-HiBiT luminescence values for the same LNP dose were plotted. See FIG. 29. The plot on the left depicts luminescence values for each gRNA at the 0.11 μg/mL dose from screen 1 (x-axis) and screen 2 (y-axis). The plot on the right depicts luminescence values for each gRNA at the 0.33 μg/mL dose from screen 1 (x-axis) and screen 2 (y-axis). Pearson correlation coefficients (PCC) and statistical significance (p) are indicated. The data demonstrated strong correlation between screens performed on different days, and demonstrated that the data sets are robust.

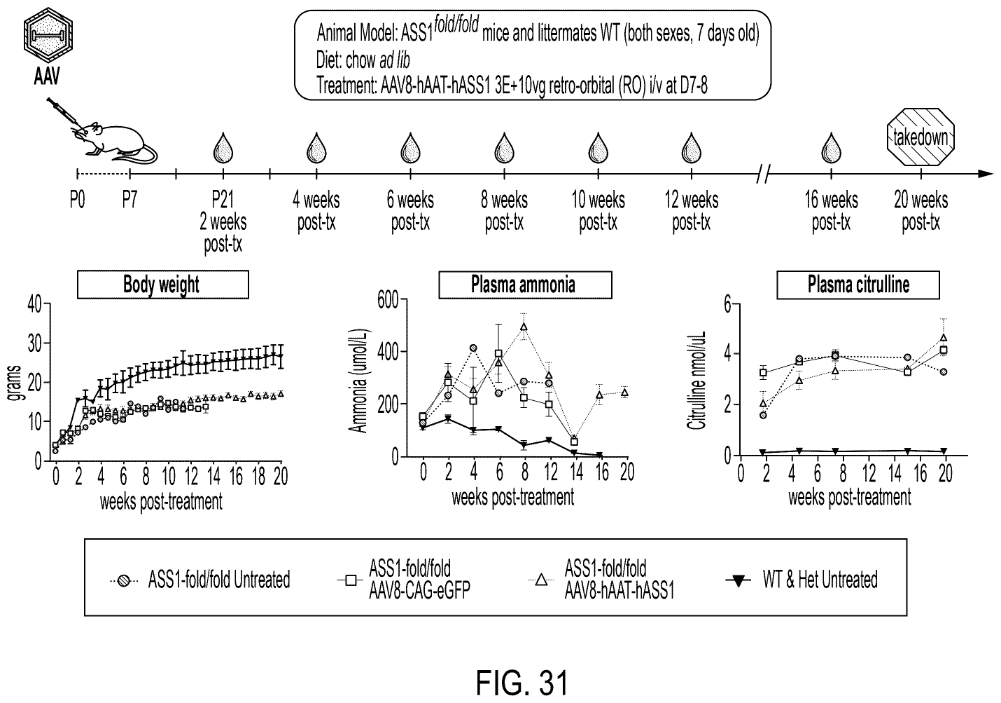

To determine the indel frequency correlation between screens, indel frequencies from screens 1 and 2 for each gRNA were plotted. See FIG. 30. Each dot on the scatter plot indicates the indel frequency for an individual gRNA from screen 1 (x-axis) and screen 2 (y-axis). Pearson correlation coefficients (PCC) and statistical significance (p) are indicated. The data demonstrated strong correlation between screens performed on different days, and demonstrated that the data sets are robust.

A summary table is shown in Table 23 indicating the top 30 ASS1 intron 1 gRNAs as determined by their ASS1-HiBiT luminescence values. Indel frequency is also denoted.

TABLE 23
Human ASS1 intron 1 guide RNA screen data summary.

| | Screen 1 | Screen 2 | Screen 1 | Screen 2 | | |
|---|---|---|---|---|---|---|
| | RLUs 0.11 | RLUs 0.11 | RLUs 0.33 | RLUs 0.33 | Screen 1% | Screen 2% |
| Guide | μg/mL LNP | μg/mL LNP | μg/mL LNP | μg/mL LNP | indel | indel |
| G033118 | 3608.33 | 2367.00 | 4378.00 | 2461.00 | 44.70 | 32.69 |
| G033111 | 2921.33 | 1532.67 | 4313.33 | 1860.00 | 50.93 | 50.68 |
| G033154 | 1949.67 | 971.50 | 3065.00 | 1122.33 | 25.35 | 23.38 |
| G033149 | 1784.33 | 989.33 | 3069.00 | 1331.00 | 27.67 | 24.73 |
| G033140 | 1129.67 | 1227.00 | 2054.33 | 1453.67 | 58.63 | 51.57 |
| G033172 | 993.00 | 1406.33 | 2083.33 | 2223.00 | 60.88 | 52.57 |
| G033163 | 1137.00 | 960.33 | 1856.33 | 1518.33 | 61.30 | 56.13 |
| G033112 | 1763.00 | 727.00 | 3414.33 | 1061.33 | 23.18 | 16.25 |
| G033133 | 1467.67 | 741.33 | 1719.67 | 1068.67 | 44.36 | 42.38 |
| G033126 | 759.33 | 1241.33 | 1334.33 | 2043.33 | 37.31 | 32.36 |
| G033129 | 2212.67 | 696.00 | 1813.33 | 1565.00 | 20.19 | 3.82 |
| G033164 | 867.00 | 1018.00 | 1349.00 | 1388.67 | 48.50 | 46.82 |
| G033093 | 1072.33 | 859.67 | 1180.33 | 993.33 | 77.88 | 71.65 |
| G033158 | 752.00 | 1158.67 | 1039.67 | 971.33 | 89.88 | 84.47 |
| G033139 | 1133.33 | 712.33 | 892.00 | 773.33 | 38.26 | 26.93 |
| G033087 | 705.33 | 1144.00 | 1878.00 | 1558.00 | 17.20 | 18.24 |
| G033122 | 3162.33 | 503.67 | 3083.33 | 931.67 | 30.67 | 33.58 |
| G033109 | 593.67 | 1183.67 | 1374.00 | 1500.00 | 41.57 | 40.39 |
| G033169 | 1093.67 | 687.33 | 1086.67 | 827.33 | 79.47 | 75.41 |
| G033144 | 1083.00 | 608.00 | 1982.67 | 957.00 | 37.33 | 35.90 |
| G033103 | 1266.33 | 475.00 | 2227.00 | 651.33 | 18.85 | 15.57 |
| G033096 | 662.00 | 845.33 | 1723.33 | 1975.00 | 27.90 | 23.61 |
| G033150 | 474.67 | 1090.00 | 1115.00 | 1723.33 | 9.93 | 7.80 |
| G033142 | 615.33 | 867.00 | 1212.33 | 1180.00 | 78.93 | 71.61 |
| G033168 | 1601.00 | 446.33 | 2309.67 | 870.33 | 39.00 | 34.30 |
| G033152 | 651.33 | 824.00 | 2147.67 | 1165.67 | 73.50 | 63.44 |
| G033116 | 1065.00 | 482.00 | 1360.00 | 593.67 | 82.47 | 74.71 |
| G033123 | 993.00 | 493.00 | 2068.67 | 881.67 | 45.17 | 43.14 |
| G033171 | 1676.33 | 356.00 | 1374.33 | 593.33 | 77.65 | 71.54 |
| G033120 | 737.67 | 680.00 | 1432.00 | 1619.00 | 15.08 | 16.73 |

### Example 7. Optimized ASS1 Insertion Constructs

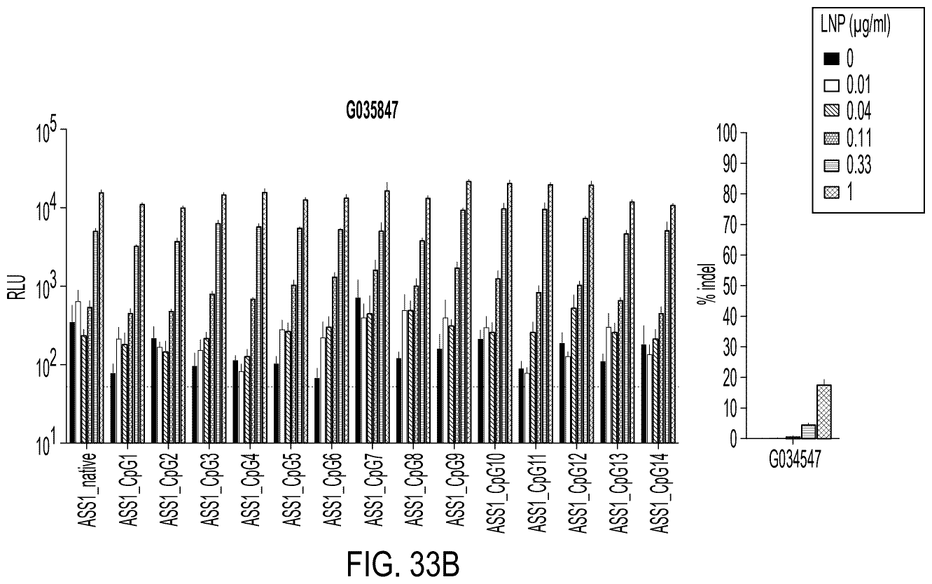

A series of CpG-depleted bidirectional ASS1 intron 2 insertion templates (ASS1_CpG #1-14) were designed and evaluated for comparability to the non-CpG-depleted insertion template pAAV-ASS1-v4 (labeled “ASS1_native”) that was used during gRNA screening in Example 5. HiBiT-tagged ASS1 intron 2 insertion templates were evaluated using the in vitro human hepatocyte gene insertion assay described in Example 5. Each insertion template was evaluated using 2 independent ASS1 intron 2 gRNAs (G035838, G035847). Insertion-derived ASS1-HiBiT luminescence was measured on day 7 post-treatment. HiBiT luminescence measurements indicated all 14 CpG-depleted intron 2 insertion templates yielded comparable ASS1 protein expression, as measured by HiBiT luminescence, relative to the non-CpG-depleted insertion template pAAV-ASS1-v4 (labeled “ASS1_native”). See FIGS. 33A-33B.

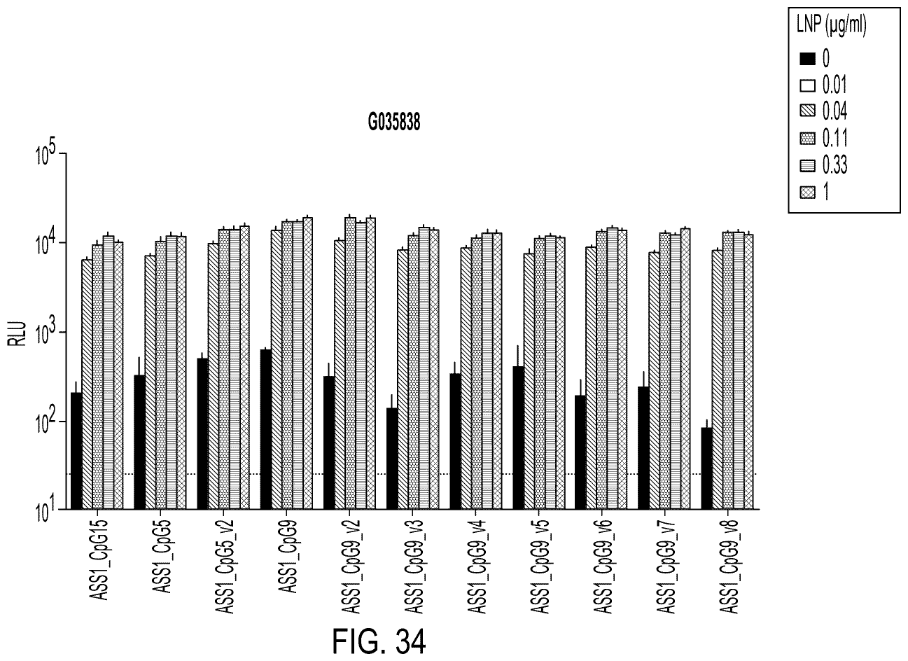

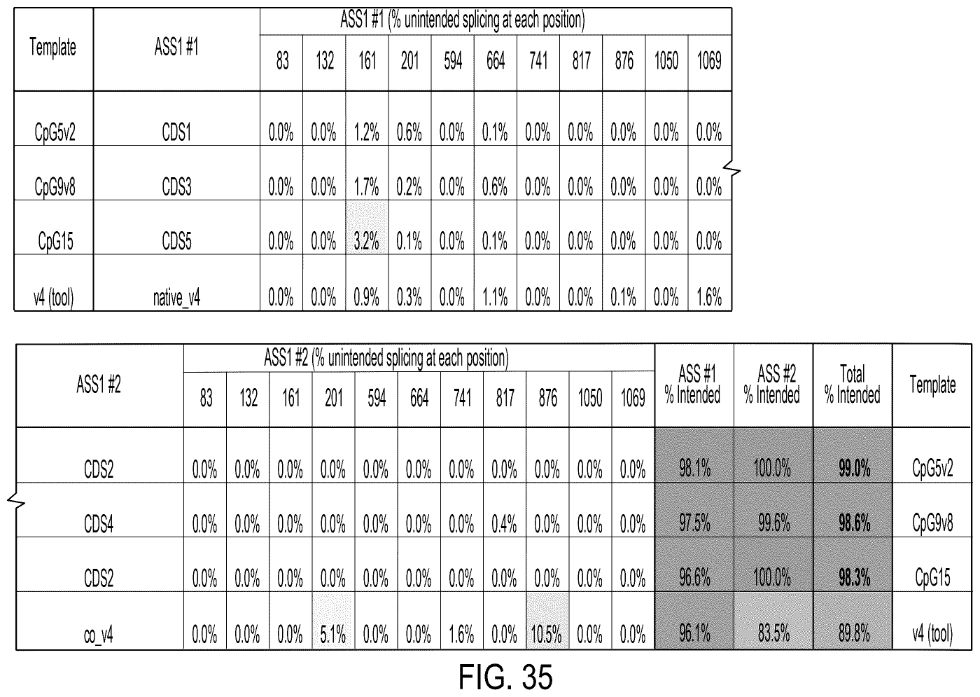

RNA sequencing was conducted on RNA extracts from human hepatocytes having undergone in vitro gene insertion with the CpG-depleted ASS1 intron 2 insertion templates, and the parental non-CpG-depleted pAAV-ASS1-v4 template to determine the frequency of mis-splicing throughout the ASS1 coding sequence transgene. This revealed that the parental non-CpG-depleted pAAV-ASS1-v4 template used in Example 5 possessed a mis-splicing frequency of approximately 10.2%, owing largely to mis-splicing at positions 201 and 876 of ASS1 cDNA #2 (co_v4). See FIG. 35. CpG-depleted intron 2 insertion template constructs CpG5 and CpG9 were selected for site-directed mutagenesis to inactivate cryptic splicing motifs uncovered through this analysis. This led to one new CpG-depleted template derived from construct Cpg5 (labeled CpG5v2) and 7 new CpG-depleted templates derived from construct CpG9 (labeled CpG9v2-8). Additionally, a new construct labeled CpG15 was designed using a previously untested combination of CpG-depleted ASS1 cDNA sequences. These new insertion templates were evaluated in the in vitro human hepatocyte gene insertion assay described above for comparability to constructs CpG5 and CpG9. HiBiT luminescence measurements indicated constructs CpG5v2, CpG9v2-8, and CpG15 yielded comparable ASS1 protein expression, as measured by HiBiT luminescence, relative to constructs CpG5 and CpG9. See FIG. 34.

RNA sequencing was conducted on RNA extracts from human hepatocytes having undergone in vitro gene insertion with the CpG-depleted, mis-splicing corrected ASS1 intron 2 insertion templates to determine the frequency of mis-splicing throughout the ASS1 coding sequence transgene. The rate of mis-splicing for constructs CpG5v2 (insertion construct 55 [CpG5v2b], but with HiBiT tags), CpG9v8 (insertion construct 56 [CpG9v8b], but with HiBiT tags), and CpG15 (insertion construct 57 [CpG15b], but with HiBiT tags) was measured to be 1.0%, 1.4%, and 1.7%, respectively. See FIG. 35.

We claim:

1. A composition comprising a nuclease agent that targets a nuclease target site in an argininosuccinate synthase 1 (ASS1) gene.

2. The composition of claim 1, wherein the ASS1 gene is a human ASS1 gene.

3. The composition of claim 1 or 2, wherein the nuclease target site is in intron 1 or intron 2 of the ASS1 gene.

4. The composition of any one of claims 1-3, wherein the nuclease target site is in intron 2 of the ASS1 gene.

5. The composition of any one of claims 1-4, wherein the nuclease agent comprises:
(a) a zinc finger nuclease (ZFN);
(b) a transcription activator-like effector nuclease (TALEN); or
(c) (i) a Cas protein or a nucleic acid encoding the Cas protein; and
(ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

6. A composition comprising a guide RNA or one or more DNAs encoding the guide RNA, wherein the guide RNA comprises a DNA-targeting segment that targets a guide RNA target sequence in an intron of an ASS1 gene, and wherein the guide RNA binds to a Cas protein and targets the Cas protein to the guide RNA target sequence.

7. The composition of claim 6, wherein the intron is intron 1 or intron 2.

8. The composition of claim 6 or 7, wherein the ASS1 gene is a human ASS1 gene, and the guide RNA target sequence is in intron 2 of the human ASS1 gene.

9. The composition of any one of claims 6-8, wherein:
(I) the DNA-targeting segment comprises at least 17, at least 18, at least 19, or at least 20 contiguous nucleotides of the sequence set forth in any one of SEQ ID NOS: 78, 60, 89, 31-59, 61-77, 79-88, and 90-118, optionally wherein the DNA-targeting segment comprises at least 17, at least 18, at least 19, or at least 20 contiguous nucleotides of the sequence set forth in any one of SEQ ID NOS: 78, 60, 89, 58, 67, 73, 92, and 114, optionally wherein the DNA-targeting segment comprises at least 17, at least 18, at least 19, or at least 20 contiguous nucleotides of the sequence set forth in any one of SEQ ID NOS: 78, 60, and 89, optionally wherein the DNA-targeting segment comprises at least 17, at least 18, at least 19, or at least 20 contiguous nucleotides of the sequence set forth in SEQ ID NO: 78; and/or
(II) the DNA-targeting segment is at least 90% or at least 95% identical to the sequence set forth in any one of SEQ ID NOS: 78, 60, 89, 31-59, 61-77, 79-88, and 90-118, optionally wherein the DNA-targeting segment is at least 90% or at least 95% identical to the sequence set forth in any one of SEQ ID NOS: 78, 60, 89, 58, 67, 73, 92, and 114, optionally wherein the DNA-targeting segment is at least 90% or at least 95% identical to the sequence set forth in any one of SEQ ID NOS: 78, 60, and 89, optionally wherein the DNA-targeting segment is at least 90% or at least 95% identical to the sequence set forth in SEQ ID NO: 78.

10. The composition of any one of claims 6-9, wherein the DNA-targeting segment comprises any one of SEQ ID NOS: 78, 60, 89, 31-59, 61-77, 79-88, and 90-118, optionally wherein the DNA-targeting segment comprises any one of SEQ ID NOS: 78, 60, 89, 58, 67, 73, 92, and 114, optionally wherein the DNA-targeting segment comprises any one of SEQ ID NOS: 78, 60, and 89, optionally wherein the DNA-targeting segment comprises SEQ ID NO: 78.

11. The composition of any one of claims 6-10, wherein the DNA-targeting segment consists of any one of SEQ ID NOS: 78, 60, 89, 31-59, 61-77, 79-88, and 90-118, optionally wherein the DNA-targeting segment consists of any one of SEQ ID NOS: 78, 60, 89, 58, 67, 73, 92, and 114, optionally wherein the DNA-targeting segment consists of any one of SEQ ID NOS: 78, 60, and 89, optionally wherein the DNA-targeting segment consists of SEQ ID NO: 78.

12. The composition of any one of claims 6-11, wherein the guide RNA comprises any one of SEQ ID NOS: 387, 369, 398, 340-368, 370-386, 388-397, and 399-427, optionally wherein the guide RNA comprises any one of SEQ ID NOS: 387, 369, 398, 367, 376, 382, 401, and 423, optionally wherein the guide RNA comprises any one of SEQ ID NOS: 387, 369, and 398, optionally wherein the guide RNA comprises SEQ ID NO: 387.

13. The composition of any one of claims 6-12, wherein:
(I) the DNA-targeting segment comprises at least 17, at least 18, at least 19, or at least 20 contiguous nucleotides of SEQ ID NO: 78; and/or
(II) the DNA-targeting segment is at least 90% or at least 95% identical to SEQ ID NO: 78.

14. The composition of any one of claims 6-13, wherein the DNA-targeting segment comprises SEQ ID NO: 78.

15. The composition of any one of claims 6-14, wherein the DNA-targeting segment consists of SEQ ID NO: 78.

16. The composition of any one of claims 6-11 and 13-15, wherein the guide RNA comprises SEQ ID NO: 387.

17. The composition of claim 6 or 7, wherein the ASS1 gene is a human ASS1 gene, and the guide RNA target sequence is in intron 1 of the human ASS1 gene.

18. The composition of any one of claims 6, 7, and 17, wherein:
(I) the DNA-targeting segment comprises at least 17, at least 18, at least 19, or at least 20 contiguous nucleotides of the sequence set forth in any one of SEQ ID NOS: 430-517; and/or
(II) the DNA-targeting segment is at least 90% or at least 95% identical to the sequence set forth in any one of SEQ ID NOS: 430-517.

19. The composition of any one of claims 6, 7, 17, and 18, wherein the DNA-targeting segment comprises any one of SEQ ID NOS: 430-517.

20. The composition of any one of claims 6, 7, and 17-19, wherein the DNA-targeting segment consists of any one of SEQ ID NOS: 430-517.

21. The composition of any one of claims 6, 7, and 17-20, wherein the guide RNA comprises any one of SEQ ID NOS: 606-693.

22. The composition of any one of claims 6-21, wherein the composition comprises the guide RNA in the form of RNA.

23. The composition of any one of claims 6-22, wherein the guide RNA comprises at least one modification.

24. The composition of claim 23, wherein the at least one modification comprises a 2′-O-methyl-modified nucleotide.

25. The composition of claim 23 or 24, wherein the at least one modification comprises a phosphorothioate bond between nucleotides.

26. The composition of any one of claims 23-25, wherein the at least one modification comprises a modification at one or more of the first five nucleotides at the 5′ end of the guide RNA.

27. The composition of any one of claims 23-26, wherein the at least one modification comprises a modification at one or more of the last five nucleotides at the 3′ end of the guide RNA.

28. The composition of any one of claims 23-27, wherein the at least one modification comprises phosphorothioate bonds between the first four nucleotides at the 5′ end of the guide RNA.

29. The composition of any one of claims 23-28, wherein the at least one modification comprises phosphorothioate bonds between the last four nucleotides at the 3′ end of the guide RNA.

30. The composition of any one of claims 23-29, wherein the at least one modification comprises 2′-O-methyl-modified nucleotides at the first three nucleotides at the 5′ end of the guide RNA.

31. The composition of any one of claims 23-30, wherein the at least one modification comprises 2′-O-methyl-modified nucleotides at the last three nucleotides at the 3′ end of the guide RNA.

32. The composition of any one of claims 23-31, wherein the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

33. The composition of any one of claims 6-32, wherein the guide RNA is a single guide RNA (sgRNA).

34. The composition of claim 33, wherein the guide RNA has the modification pattern set forth in SEQ ID NO: 429.

35. The composition of any one of claims 6-16 and 22-34, wherein the composition comprises the guide RNA in the form of RNA, the guide RNA comprises SEQ ID NO: 387, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

36. The composition of claim 35, wherein the composition comprises the guide RNA in the form of RNA, the guide RNA comprises SEQ ID NO: 387, and the guide RNA has the modification pattern set forth in SEQ ID NO: 429.

37. The composition of any one of claims 6-36, wherein the Cas protein is a Cas9 protein.

38. The composition of claim 37, wherein the Cas protein is derived from a Streptococcus pyogenes Cas9 protein.

39. The composition of any one of claims 6-38, wherein the Cas protein comprises the sequence set forth in SEQ ID NO: 12.

40. The composition of any one of claims 6-39, further comprising the Cas protein or a nucleic acid encoding the Cas protein.

41. The composition of claim 40, wherein the nucleic acid encoding the Cas protein is codon-optimized for expression in a mammalian cell or a human cell.

42. The composition of claim 40 or 41, wherein the composition comprises the nucleic acid encoding the Cas protein, wherein the nucleic acid comprises an mRNA encoding the Cas protein.

43. The composition of claim 42, wherein the mRNA encoding the Cas protein comprises at least one modification.

44. The composition of claim 42 or 43, wherein the mRNA encoding the Cas protein is modified to comprise a modified uridine at one or more or all uridine positions.

45. The composition of claim 44, wherein the modified uridine is N1-methyl-pseudouridine.

46. The composition of claim 44 or 45, wherein the mRNA encoding the Cas protein is fully substituted with N1-methyl-pseudouridine.

47. The composition of any one of claims 42-46, wherein the mRNA encoding the Cas protein comprises a 5′ cap.

48. The composition of any one of claims 42-47, wherein the mRNA encoding the Cas protein comprises a poly(A) tail.

49. The composition of any one of claims 42-48, wherein the mRNA encoding the Cas protein comprises the sequence set forth in SEQ ID NO: 250 or 249.

50. The composition of any one of claims 40-49, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail.

51. The composition of any one of claims 40-50, wherein the composition comprises the guide RNA in the form of RNA, and the guide RNA comprises SEQ ID NO: 387, and
wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 250 or 249.

52. The composition of any one of claims 40-51, wherein the composition comprises the guide RNA in the form of RNA, the guide RNA comprises SEQ ID NO: 387, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and
wherein the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail.

53. The composition of any one of claims 40-52, wherein the composition comprises the guide RNA in the form of RNA, the guide RNA comprises SEQ ID NO: 387, and the guide RNA has the modification pattern set forth in SEQ ID NO: 429, and
wherein the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail.

54. The composition of any one of claims 40-53, wherein the Cas protein or the nucleic acid encoding the Cas protein and the guide RNA or the one or more DNAs encoding the guide RNA are associated with a lipid nanoparticle.

55. The composition of claim 54, wherein the lipid nanoparticle comprises a cationic lipid, a neutral lipid, a helper lipid, and a stealth lipid.

56. The composition of claim 55, wherein the cationic lipid is Lipid A.

57. The composition of claim 55 or 56, wherein the neutral lipid is DSPC.

58. The composition of any one of claims 55-57, wherein the helper lipid is cholesterol.

59. The composition of any one of claims 55-58, wherein the stealth lipid is 1,2-dimyristoyl-rac-glycero-3-methoxypolyethylene glycol-2000 (PEG2k-DMG).

60. The composition of any one of claims 55-59, wherein the cationic lipid is Lipid A, the neutral lipid is DSPC, the helper lipid is cholesterol, and the stealth lipid is PEG2k-DMG.

61. The composition of any one of claims 55-60, wherein the lipid nanoparticle comprises four lipids at the following molar ratios: about 50 mol % Lipid A, about 9 mol % DSPC, about 38 mol % cholesterol, and about 3 mol % PEG2k-DMG.

62. The composition of any one of claims 40-61, wherein the ASS1 gene is a human ASS1 gene,
wherein the composition comprises the guide RNA in the form of RNA, and the guide RNA comprises SEQ ID NO: 387,
wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 250 or 249, and
wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG.

63. The composition of any one of claims 40-62, wherein the ASS1 gene is a human ASS1 gene,
wherein the composition comprises the guide RNA in the form of RNA, the guide RNA comprises SEQ ID NO: 387, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA,
wherein the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail, and
wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG.

64. The composition of any one of claims 40-63, wherein the ASS1 gene is a human ASS1 gene,
wherein the composition comprises the guide RNA in the form of RNA, the guide RNA comprises SEQ ID NO: 387, and the guide RNA has the modification pattern set forth in SEQ ID NO: 429,
wherein the composition comprises the nucleic acid encoding the Cas protein, wherein the nucleic acid comprises an mRNA encoding the Cas protein, the mRNA encoding the Cas protein comprises the sequence set forth in SEQ ID NO: 250 or 249, and the mRNA encoding the Cas protein is fully substituted with N1-methyl-pseudouridine, comprises a 5′ cap, and comprises a poly(A) tail, and
wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG.

65. A composition comprising a nucleic acid construct comprising a first argininosuccinate synthase protein coding sequence.

66. The composition of claim 65, wherein the first argininosuccinate synthase protein coding sequence is a human argininosuccinate synthase protein coding sequence.

67. The composition of claim 65 or 66, wherein the first argininosuccinate synthase protein coding sequence comprises human argininosuccinate synthase exons 2-14 or exons 3-14.

68. The composition of any one of claims 65-67, wherein the first argininosuccinate synthase protein coding sequence comprises human argininosuccinate synthase exons 3-14.

69. The composition of any one of claims 65-68, wherein the first argininosuccinate synthase protein coding sequence encodes a protein at last 95%, at least 96%, at least 97%, at least 98%, or at least 99% identical to the sequence set forth in SEQ ID NO: 230.

70. The composition of any one of claims 65-69, wherein the first argininosuccinate synthase protein coding sequence encodes a protein comprising the sequence set forth in SEQ ID NO: 230.

71. The composition of any one of claims 65-70, wherein the first argininosuccinate synthase protein coding sequence encodes a protein consisting of the sequence set forth in SEQ ID NO: 230.

72. The composition of any one of claims 65-71, wherein the first argininosuccinate synthase protein coding sequence is at least 96%, 97%, 98%, or 99% identical to any one of SEQ ID NOS: 705, 709, and 713.

73. The composition of any one of claims 65-71, wherein the first argininosuccinate synthase protein coding sequence is at least 90%, 95%, 96%, 97%, 98%, or 99% identical to any one of SEQ ID NOS: 706 and 710.

74. The composition of any one of claims 65-71, wherein the first argininosuccinate synthase protein coding sequence is at least 99% identical to any one of SEQ ID NOS: 705, 706, 709, 710, and 713.

75. The composition of any one of claims 65-71, wherein the first argininosuccinate synthase protein coding sequence comprises, consists essentially of, or consists of any one of SEQ ID NOS: 705, 706, 709, 710, and 713.

76. The composition of any one of claims 65-71, wherein the first argininosuccinate synthase protein coding sequence is at least 96%, 97%, 98%, or 99% identical to SEQ ID NO: 705.

77. The composition of any one of claims 65-71, wherein the first argininosuccinate synthase protein coding sequence is at least 99% identical to SEQ ID NO: 705.

78. The composition of any one of claims 65-71, wherein the first argininosuccinate synthase protein coding sequence comprises, consists essentially of, or consists of SEQ ID NO: 705.

79. The composition of any one of claims 65-71, wherein the first argininosuccinate synthase protein coding sequence is at least 90%, 95%, 96%, 97%, 98%, or 99% identical to SEQ ID NO: 706.

80. The composition of any one of claims 65-71, wherein the first argininosuccinate synthase protein coding sequence is at least 99% identical to SEQ ID NO: 706.

81. The composition of any one of claims 65-71, wherein the first argininosuccinate synthase protein coding sequence comprises, consists essentially of, or consists of SEQ ID NO: 706.

82. The composition of any one of claims 65-67, wherein the first argininosuccinate synthase protein coding sequence comprises human argininosuccinate synthase exons 2-14.

83. The composition of any one of claims 65-67 and 82, wherein the first argininosuccinate synthase protein coding sequence encodes a protein at last 95%, at least 96%, at least 97%, at least 98%, or at least 99% identical to the sequence set forth in SEQ ID NO: 227.

84. The composition of any one of claims 65-67, 82, and 83, wherein the first argininosuccinate synthase protein coding sequence encodes a protein comprising the sequence set forth in SEQ ID NO: 227.

85. The composition of any one of claims 65-67 and 82-84, wherein the first argininosuccinate synthase protein coding sequence encodes a protein consisting of the sequence set forth in SEQ ID NO: 227.

86. The composition of any one of claims 65-85, wherein the nucleic acid construct comprises a splice acceptor upstream of the first argininosuccinate synthase protein coding sequence.

87. The composition of any one of claims 65-86, wherein the nucleic acid construct comprises a polyadenylation signal downstream of the first argininosuccinate synthase protein coding sequence.

88. The composition of any one of claims 65-85, wherein the nucleic acid construct comprises a splice acceptor upstream of the first argininosuccinate synthase protein coding sequence, and the nucleic acid construct comprises a polyadenylation signal downstream of the first argininosuccinate synthase protein coding sequence.

89. The composition of any one of claims 65-88, wherein the nucleic acid construct does not comprise homology arms.

90. The composition of any one of claims 65-88, wherein the nucleic acid construct comprises homology arms.

91. The composition of any one of claims 65-90, wherein the nucleic acid construct does not comprise a promoter that drives expression of the argininosuccinate synthase protein.

92. The composition of any one of claims 65-91, wherein the nucleic acid construct is a bidirectional construct.

93. The composition of claim 92, wherein the nucleic acid construct comprises the first argininosuccinate synthase protein coding sequence and a reverse complement of a second argininosuccinate synthase protein coding sequence.

94. The composition of claim 93, wherein the first argininosuccinate synthase protein coding sequence and the second argininosuccinate synthase protein coding sequence are different but encode the same argininosuccinate synthase protein sequence.

95. The composition of claim 93 or 94, wherein the first argininosuccinate synthase protein coding sequence and the second argininosuccinate synthase protein coding sequence each encode a protein at least 95%, at least 96%, at least 97%, at least 98%, or at least 99% identical to the sequence set forth in SEQ ID NO: 230.

96. The composition of any one of claims 93-95, wherein the first argininosuccinate synthase protein coding sequence and the second argininosuccinate synthase protein coding sequence each encode a protein comprising the sequence set forth in SEQ ID NO: 230.

97. The composition of any one of claims 93-96, wherein the first argininosuccinate synthase protein coding sequence and the second argininosuccinate synthase protein coding sequence each encode a protein consisting of the sequence set forth in SEQ ID NO: 230.

98. The composition of any one of claims 93-97, wherein the first argininosuccinate synthase protein coding sequence is at least 96%, 97%, 98%, or 99% identical to any one of SEQ ID NOS: 705, 709, and 713, optionally wherein the first argininosuccinate synthase protein coding sequence is at least 96%, 97%, 98%, or 99% identical to SEQ ID NO: 705.

99. The composition of any one of claims 93-98, wherein the first argininosuccinate synthase protein coding sequence is at least 99% identical to any one of SEQ ID NOS: 705, 709, and 713, optionally wherein the first argininosuccinate synthase protein coding sequence is at least 99% identical to SEQ ID NO: 705.

100. The composition of any one of claims 93-99, wherein the first argininosuccinate synthase protein coding sequence comprises, consists essentially of, or consists of any one of SEQ ID NOS: 705, 709, and 713, optionally wherein the first argininosuccinate synthase protein coding sequence comprises, consists essentially of, or consists of SEQ ID NO: 705.

101. The composition of any one of claims 93-100, wherein the second argininosuccinate synthase protein coding sequence is at least 90%, 95%, 96%, 97%, 98%, or 99% identical to any one of SEQ ID NOS: 706 and 710, optionally wherein the second argininosuccinate synthase protein coding sequence is at least 90%, 95%, 96%, 97%, 98%, or 99% identical to SEQ ID NO: 706.

102. The composition of any one of claims 93-101, wherein the second argininosuccinate synthase protein coding sequence is at least 99% identical to any one of SEQ ID NOS: 706 and 710, optionally wherein the second argininosuccinate synthase protein coding sequence is at least 99% identical to SEQ ID NO: 706.

103. The composition of any one of claims 93-102, wherein the second argininosuccinate synthase protein coding sequence comprises, consists essentially of, or consists of any one of SEQ ID NOS: 706 and 710, optionally wherein the second argininosuccinate synthase protein coding sequence comprises, consists essentially of, or consists of SEQ ID NO: 706.

104. The composition of any one of claims 93-97, wherein the first argininosuccinate synthase protein coding sequence is at least 96%, 97%, 98%, or 99% identical to SEQ ID NO: 705, and wherein the second argininosuccinate synthase protein coding sequence is at least 90%, 95%, 96%, 97%, 98%, or 99% identical to SEQ ID NO: 706.

105. The composition of any one of claims 93-97 and 104, wherein the first argininosuccinate synthase protein coding sequence is at least 99% identical to SEQ ID NO: 705, and wherein the second argininosuccinate synthase protein coding sequence is at least 99% identical to SEQ ID NO: 706.

106. The composition of any one of claims 93-97, 104, and 105, wherein the first argininosuccinate synthase protein coding sequence comprises, consists essentially of, or consists of SEQ ID NO: 705, and wherein the second argininosuccinate synthase protein coding sequence comprises, consists essentially of, or consists of SEQ ID NO: 706.

107. The composition of claim 93 or 94, wherein the first argininosuccinate synthase protein coding sequence and the second argininosuccinate synthase protein coding sequence each encode a protein at least 95%, at least 96%, at least 97%, at least 98%, or at least 99% identical to the sequence set forth in SEQ ID NO: 227.

108. The composition of any one of claims 93, 94, and 107, wherein the first argininosuccinate synthase protein coding sequence and the second argininosuccinate synthase protein coding sequence each encode a protein comprising the sequence set forth in SEQ ID NO: 227.

109. The composition of any one of claims 93, 94, 107, and 108, wherein the first argininosuccinate synthase protein coding sequence and the second argininosuccinate synthase protein coding sequence each encode a protein consisting of the sequence set forth in SEQ ID NO: 227.

110. The composition of any one of claim 93-109, wherein the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor, or wherein the nucleic acid construct comprises from 5′ to 3′: a first splice acceptor, the second argininosuccinate synthase protein coding sequence, a first polyadenylation signal, a reverse complement of a second polyadenylation signal, the reverse complement of the first argininosuccinate synthase protein coding sequence, and a reverse complement of a second splice acceptor.

111. The composition of claim 110, wherein the first argininosuccinate synthase protein coding sequence and the second argininosuccinate synthase protein coding sequence are different but encode the same argininosuccinate synthase protein sequence, and wherein the first polyadenylation signal and the second polyadenylation signal are different.

112. The composition of any one of claims 93-106, wherein the first argininosuccinate synthase protein coding sequence and the second argininosuccinate synthase protein coding sequence are different and each encode a protein comprising or consisting of the sequence set forth in SEQ ID NO: 230,
wherein the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor,
wherein the nucleic acid construct does not comprise a promoter that drives expression of the argininosuccinate synthase protein, and
wherein the nucleic acid construct does not comprise homology arms.

113. The composition of any one of claims 93-106, wherein the first argininosuccinate synthase protein coding sequence and the second argininosuccinate synthase protein coding sequence are different and each encode a protein comprising or consisting of the sequence set forth in SEQ ID NO: 230,
wherein the first argininosuccinate synthase protein coding sequence comprises SEQ ID NO: 705,
wherein the second argininosuccinate synthase protein coding sequence comprises SEQ ID NO: 706,
wherein the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor,
wherein the nucleic acid construct does not comprise a promoter that drives expression of the argininosuccinate synthase protein, and
wherein the nucleic acid construct does not comprise homology arms.

114. The composition of any one of claims 93-108 and 110-113, wherein the first argininosuccinate synthase protein coding sequence and the second argininosuccinate synthase protein coding sequence are different and each encode a protein comprising or consisting of the sequence set forth in SEQ ID NO: 227,
wherein the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor,
wherein the nucleic acid construct does not comprise a promoter that drives expression of the argininosuccinate synthase protein, and
wherein the nucleic acid construct does not comprise homology arms.

115. The composition of any one of claims 65-91, wherein the nucleic acid construct is a unidirectional construct.

116. The composition of claim 115, wherein the nucleic acid construct is a unidirectional construct comprising the first argininosuccinate synthase protein coding sequence,
wherein the first argininosuccinate synthase protein coding sequence encodes a protein comprising or consisting of the sequence set forth in SEQ ID NO: 230,
wherein the nucleic acid construct comprises from 5′ to 3′: a splice acceptor, the first argininosuccinate synthase protein coding sequence, and a polyadenylation signal,
wherein the nucleic acid construct does not comprise a promoter that drives expression of the argininosuccinate synthase protein, and
wherein the nucleic acid construct does not comprise homology arms.

117. The composition of claim 115, wherein the nucleic acid construct is a unidirectional construct comprising the first argininosuccinate synthase protein coding sequence,
wherein the first argininosuccinate synthase protein coding sequence encodes a protein comprising or consisting of the sequence set forth in SEQ ID NO: 227,
wherein the nucleic acid construct comprises from 5′ to 3′: a splice acceptor, the first argininosuccinate synthase protein coding sequence, and a polyadenylation signal,
wherein the nucleic acid construct does not comprise a promoter that drives the expression of the argininosuccinate synthase protein, and
wherein the nucleic acid construct does not comprise homology arms.

118. The composition of any one of claims 65-117, wherein the nucleic acid construct is single-stranded DNA or double-stranded DNA.

119. The composition of claim 118, wherein the nucleic acid construct is single-stranded DNA.

120. The composition of any one of claims 65-119, wherein the nucleic acid construct is in a nucleic acid vector or a lipid nanoparticle.

121. The composition of claim 120, wherein the nucleic acid construct is in the nucleic acid vector, optionally wherein the nucleic acid vector is a viral vector.

122. The composition of claim 121, wherein the nucleic acid vector is an adeno-associated viral (AAV) vector,
optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196.

123. The composition of claim 122, wherein the AAV vector is a single-stranded AAV (ssAAV) vector.

124. The composition of claim 122 or 123, wherein the AAV vector is derived from an AAV8 vector, an AAV3B vector, an AAV5 vector, an AAV6 vector, an AAV7 vector, an AAV9 vector, an AAVrh.74 vector, or an AAVhu.37 vector, optionally wherein the AAV vector is a recombinant AAV8 (rAAV8) or a recombinant AAV5 (rAAV5) vector.

125. The composition of claim 124, wherein the AAV vector is a recombinant AAV8 (rAAV8) vector.

126. The composition of claim 125, wherein the AAV vector is a single-stranded rAAV8 vector.

127. The composition of any one of claims 93-111, wherein the first argininosuccinate synthase protein coding sequence and the second argininosuccinate synthase protein coding sequence are different and each encode a protein comprising or consisting of the sequence set forth in SEQ ID NO: 230,
wherein the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor,
wherein the nucleic acid construct does not comprise a promoter that drives expression of the argininosuccinate synthase protein,
wherein the nucleic acid construct does not comprise homology arms, and
wherein the nucleic acid construct is in a single-stranded rAAV8 vector,
optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196.

128. The composition of any one of claims 93-111, wherein the first argininosuccinate synthase protein coding sequence and the second argininosuccinate synthase protein coding sequence are different and each encode a protein comprising or consisting of the sequence set forth in SEQ ID NO: 230,
wherein the first argininosuccinate synthase protein coding sequence comprises SEQ ID NO: 705,
wherein the second argininosuccinate synthase protein coding sequence comprises SEQ ID NO: 706,
wherein the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor,
wherein the nucleic acid construct does not comprise a promoter that drives expression of the argininosuccinate synthase protein,
wherein the nucleic acid construct does not comprise homology arms, and
wherein the nucleic acid construct is in a single-stranded rAAV8 vector,
optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196.

129. The composition of any one of claims 93-111, wherein the first argininosuccinate synthase protein coding sequence and the second argininosuccinate synthase protein coding sequence are different and each encode a protein comprising or consisting of the sequence set forth in SEQ ID NO: 227,
wherein the nucleic acid construct comprises from 5′ to 3′: a first splice acceptor, the first argininosuccinate synthase protein coding sequence, a first polyadenylation signal, a reverse complement of a second polyadenylation signal, the reverse complement of the second argininosuccinate synthase protein coding sequence, and a reverse complement of a second splice acceptor,
wherein the nucleic acid construct does not comprise a promoter that drives expression of the argininosuccinate synthase protein,
wherein the nucleic acid construct does not comprise homology arms, and
wherein the nucleic acid construct is in a single-stranded rAAV8 vector,
optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196.

130. A combination comprising:
(I) the composition of any one of claims 1-5 and 40-64; and
(II) the composition comprising the nucleic acid construct comprising the first argininosuccinate synthase protein coding sequence of any one of claims 65-129.

131. The composition of any one of claims 1-129 or the combination of claim 130 for use in a method of introducing an argininosuccinate synthase (ASS1) nucleic acid into a cell, a method of integrating an ASS1 nucleic acid construct into a target gene in a cell, or a method of expressing argininosuccinate synthase in a cell.

132. Use of the composition of any one of claims 1-129 or the combination of claim 130 in the preparation of a reagent for introducing an argininosuccinate synthase (ASS1) nucleic acid into a cell, integrating an ASS1 nucleic acid construct into a target gene in a cell, or expressing argininosuccinate synthase in a cell.

133. The composition or combination for use of claim 131 or the use of claim 132, wherein the cell is a neonatal cell.

134. The composition or combination for use of claim 133 or the use of claim 133, wherein the neonatal cell is from a human neonatal subject within 24 weeks after birth, is from a human neonatal subject within 12 weeks after birth, is from a human neonatal subject within 8 weeks after birth, is from a human neonatal subject within 4 weeks after birth, is from a human neonatal subject within 2 weeks after birth, or is from a human neonatal subject within 1 week after birth.

135. The composition or combination for use of claim 131 or the use of claim 132, wherein the cell is not a neonatal cell.

136. The composition of any one of claims 1-129 or the combination of claim 130 for use in a method of treating an argininosuccinate synthase deficiency in a subject.

137. The composition of any one of claims 1-129 or the combination of claim 130 for use in a method of treating citrullinemia type I in a subject.

138. Use of the composition of any one of claims 1-129 or the combination of claim 130 in the preparation of a medicament treating an argininosuccinate synthase deficiency in a subject.

139. Use of the composition of any one of claims 1-129 or the combination of claim 130 in the preparation of a medicament for treating citrullinemia type I in a subject.

140. The composition or combination for use of claim 136 or 137 or the use of claim 138 or 139, wherein the subject is a neonatal subject.

141. The composition or combination for use of claim 140 or the use of claim 140, wherein the neonatal subject is a human neonatal subject within 24 weeks after birth, is a human neonatal subject within 12 weeks after birth, is a human neonatal subject within 8 weeks after birth, is a human neonatal subject within 4 weeks after birth, is a human neonatal subject within 2 weeks after birth, or is a human neonatal subject within 1 week after birth.

142. The composition or combination for use of claim 136 or 137 or the use of claim 138 or 139, wherein the subject is not a neonatal subject.

143. A cell comprising the composition of any one of claims 1-129 or the combination of claim 130.

144. The cell of claim 143, wherein the nucleic acid construct is integrated into an endogenous target gene locus, and wherein argininosuccinate synthase protein is expressed from the endogenous target gene locus, or wherein the nucleic acid construct is integrated into intron 1 or intron 2 of an endogenous argininosuccinate synthase (ASS1) locus, and wherein argininosuccinate synthase protein is expressed from the endogenous ASS1 locus.

145. The cell of claim 143 or 144, wherein the cell is a human cell, optionally wherein the nucleic acid construct is integrated into intron 2 of the endogenous ASS1 locus.

146. The cell of any one of claims 143-145, wherein the cell is a liver cell.

147. The cell of claim 146, wherein the liver cell is a hepatocyte.

148. The cell of any one of claims 143-147, wherein the cell is a neonatal cell.

149. The cell of claim 148, wherein the neonatal cell is from a human neonatal subject within 24 weeks after birth, is from a human neonatal subject within 12 weeks after birth, is from a human neonatal subject within 8 weeks after birth, is from a human neonatal subject within 4 weeks after birth, is from a human neonatal subject within 2 weeks after birth, or is from a human neonatal subject within 1 week after birth.

150. The cell of any one of claims 143-147, wherein the cell is not a neonatal cell.

151. The cell of any one of claims 143-150, wherein the cell is ex vivo or in vitro.

152. The cell of any one of claims 143-150, wherein the cell is in vivo.

153. A method of introducing an argininosuccinate synthase nucleic acid into a cell, comprising administering the combination of claim 130 to the cell.

154. A method of integrating an argininosuccinate synthase nucleic acid construct into a target gene in a cell, comprising administering the combination of claim 130 to the cell,
wherein the nuclease agent cleaves the nuclease target site in the target gene to create a cleavage site, the nucleic acid construct is inserted into the cleavage site to create a modified target gene, and argininosuccinate synthase protein is expressed from the modified target gene.

155. A method of expressing argininosuccinate synthase in a cell, comprising administering the combination of claim 130 to the cell,
wherein the nuclease agent cleaves the nuclease target site in the target gene to create a cleavage site, the nucleic acid construct is inserted into the cleavage site to create a modified target gene, and argininosuccinate synthase protein is expressed from the modified target gene.

156. The method of any one of claims 153-155, wherein the nuclease agent comprises:
(a) a Cas protein or a nucleic acid encoding the Cas protein; and
(b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

157. The method of claim 156, wherein the nucleic acid construct, the Cas protein or the nucleic acid encoding the Cas protein, and the guide RNA or the one or more DNAs encoding the guide RNA are administered simultaneously.

158. The method of claim 156, wherein the nucleic acid construct is not administered simultaneously with the Cas protein or the nucleic acid encoding the Cas protein and the guide RNA or the one or more DNAs encoding the guide RNA.

159. The method of any one of claims 153-158, wherein the cell is a liver cell.

160. The method of any one of claims 153-159, wherein the cell is a hepatocyte.

161. The method of any one of claims 153-160, wherein the cell is a human cell.

162. The method of any one of claims 153-161, wherein the cell is a neonatal cell.

163. The method of claim 162, wherein the neonatal cell is from a human neonatal subject within 24 weeks after birth, a human neonatal subject within 12 weeks after birth, a human neonatal subject within 8 weeks after birth, a human neonatal subject within 4 weeks after birth, a human neonatal subject within 2 weeks after birth, or a human neonatal subject within 1 week after birth.

164. The method of any one of claims 153-163, wherein the cell is not a neonatal cell.

165. The method of any one of claims 153-164, wherein the cell is in vivo.

166. The method of any one of claims 153-164, wherein the cell is in vitro or ex vivo.

167. A method of treating an argininosuccinate synthase deficiency in a subject, comprising administering the combination of claim 130 to the subject.

168. A method of treating citrullinemia type I in a subject, comprising administering the combination of claim 130 to the subject.

169. A method of preventing or inhibiting hyperammonemia in a subject having citrullinemia type I, comprising administering the combination of claim 130 to the subject.

170. The method of any one of claims 167-169, wherein the nuclease agent comprises:
(a) a Cas protein or a nucleic acid encoding the Cas protein; and
(b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

171. The method of claim 170, wherein the nucleic acid construct, the Cas protein or the nucleic acid encoding the Cas protein, and the guide RNA or the one or more DNAs encoding the guide RNA are administered simultaneously.

172. The method of claim 170, wherein the nucleic acid construct is not administered simultaneously with the Cas protein or the nucleic acid encoding the Cas protein and the guide RNA or the one or more DNAs encoding the guide RNA.

173. The method of any one of claims 167-172, wherein the subject is a neonatal subject.

174. The method of claim 173, wherein the neonatal subject is a human neonatal subject within 24 weeks after birth, a human neonatal subject is within 12 weeks after birth, a human neonatal subject is within 8 weeks after birth, a human neonatal subject is within 4 weeks after birth, a human neonatal subject is within 2 weeks after birth, or a human neonatal subject is within 1 week after birth.

175. The method of any one of claims 167-172, wherein the subject is not a neonatal subject.

176. The method of any one of claims 167-175, wherein the subject is a human subject.

177. The method of any one of claims 167-176, wherein the method decreases plasma ammonia and/or plasma citrulline levels in the subject.

178. The method of claim 177, wherein the method reduces plasma ammonia levels to less than 200 μmol/L, less than 175 μmol/L, less than 150 μmol/L, less than 125 μmol/L, or less than 100 μmol/L, optionally wherein the reduced plasma ammonia levels are at 2 weeks, 4 weeks, 6 weeks, 8 weeks, 12 weeks, 16 weeks, 20 weeks, 6 months, 1 year, or 2 years after administering the combination.

179. The method of claim 177 or 178, wherein the method reduces plasma citrulline levels to less than 2000 μmol/L, less than 1750 μmol/L, less than 1500 μmol/L, less than 1250 μmol/L, less than 1000 μmol/L, less than 900 μmol/L, less than 800 μmol/L, less than 700 μmol/L, less than 600 μmol/L, or less than 500 μmol/L, optionally wherein the reduced plasma citrulline levels are at 2 weeks, 4 weeks, 6 weeks, 8 weeks, 12 weeks, 16 weeks, 20 weeks, 6 months, 1 year, or 2 years after administering the combination.

180. The method of any one of claims 177-179, wherein the decreased ammonia and/or plasma citrulline levels are sustained for at least 1 month, at least 2 months, at least 3 months, at least 6 months, at least 1 year, or at least 2 years after administering the combination.

181. The method of any one of claim 167-180, wherein the method further comprises assessing preexisting AAV immunity in the subject prior to administering the composition to the subject.

182. The method of claim 181, wherein the preexisting AAV immunity is preexisting AAV8 immunity.

183. The method of claim 181 or 182, wherein assessing preexisting AAV immunity comprises assessing immunogenicity using a total antibody immune assay or a neutralizing antibody assay.
